(19) United States
(12) Patent Application Publication
(10) Pub. No.: US 2025/0376674 A1
(43) Pub. Date: Dec. 11, 2025

(54) SPLIT PRIME EDITORS

(71) Applicant: Prime Medicine, Inc., Cambridge, MA (US)

(72) Inventors: Andrew V. Anzalone, Cambridge, MA (US); Christopher Wilson, Waltham, MA (US)

(21) Appl. No.: 18/877,108

(22) PCT Filed: Jun. 23, 2023
(86) PCT No.: PCT/US2023/026128

§ 371 Date: Dec. 19, 2024

Related U.S. Application Data
(60) Provisional application No. 63/354,844, filed on Jun. 23, 2022.

(51) Int. Cl. C12N 15/10; C12N 9/12; C12N 9/22; C12N 15/11; C12N 15/86

(52) U.S. Cl. CPC C12N 15/102; C12N 9/1276; C12N 9/226; C12N 15/111; C12N 15/86; C07K 2319/09; C07K 2319/30; C12N 2750/14143; C12N 2840/445

(57) ABSTRACT

Provided herein are compositions and methods related split prime editors.

### CROSS REFERENCE TO RELATED APPLICATIONS

This application is a § 371 national-stage application based on PCT/US23/26128, filed Jun. 23, 2023, which claims the benefit of U.S. Provisional Application No. 63/354,844, filed Jun. 23, 2022, the entire contents of each are hereby incorporated by reference.

### REFERENCE TO A SEQUENCE LISTING

This application contains a Sequence Listing which has been submitted electronically in XML format. The Sequence Listing XML is incorporated herein by reference. Said XML file, created on Jul. 24, 2023, is named PMB-00525_SL.xml and is 2,648,524 bytes in size.

### BACKGROUND

Prime editing is a gene editing technology that allows researchers to make nucleotide substitutions, insertions, deletions, or combinations thereof in the DNA of cells. Prime editing can be used to correct disease associated gene mutations, and can be used for treating disease with a genetic component. There is a need for split prime editors that have desirable properties, such as the ability to facilitate prime editing with improved efficiency.

### SUMMARY

Provided herein are split prime editors useful in prime editing, as well as methods of using and making such split prime editors.

In certain aspects, prime editor systems comprise a split prime editor comprising a DNA binding domain and a DNA polymerase domain, wherein the split prime editor comprises a first polypeptide comprising a first amino acid sequence and a second polypeptide comprising a second amino acid sequence.

In some embodiments, the first amino acid sequence forms at least a portion of the DNA binding domain. In some embodiments, the second amino acid sequence forms at least a portion of the DNA polymerase domain. In some embodiments, the first amino acid sequence forms the DNA binding domain. In some embodiments, the first amino acid sequence forms the DNA binding domain and a portion of the DNA polymerase domain. In some embodiments, the second amino acid sequence forms the DNA polymerase domain. In some embodiments, the second amino acid sequence forms the DNA polymerase domain and a portion of the DNA binding domain.

In some embodiments, the first amino acid sequence forms at least a portion of the DNA polymerase domain. In some embodiments, the second amino acid sequence forms at least a portion of the DNA binding domain. In some embodiments, the first amino acid sequence forms the DNA polymerase domain. In some embodiments, the first amino acid sequence forms the DNA polymerase domain and a portion of the DNA binding domain. In some embodiments, the second amino acid sequence forms the DNA binding domain. In some embodiments, the second amino acid sequence forms the DNA binding domain and a portion of the DNA polymerase domain.

In certain embodiments, the first polypeptide and the second polypeptide are configured to passively assemble in a host cell to form the split prime editor. In some embodiments, the first polypeptide has affinity for the second polypeptide. In some embodiments, the second polypeptide has affinity for the first polypeptide.

In some embodiments, the first polypeptide comprises a single-domain antibody (e.g., a single-domain antibody comprising an amino acid sequence as set forth in Table 17). In certain embodiments, the single-domain antibody is a NANOBODY®. In some embodiments, the second polypeptide comprises a peptide tag that is configured to be bound by the single domain antibody. In certain embodiments, the peptide tag comprises a SpotTag® or a BC2 tag. In some embodiments, the peptide tag comprises an amino acid sequence as set forth in Table 16.

In certain embodiments, the first polypeptide comprises a peptide tag that is configured to be bound by a single domain antibody. In some embodiments, the peptide tag comprises a SpotTag® or a BC2 tag. In some embodiments, the peptide tag comprises an amino acid sequence as set forth in Table 16. In certain embodiments, the second polypeptide comprises a single-domain antibody (e.g., a single-domain antibody comprising an amino acid sequence as set forth in Table 17). In certain embodiments, the single-domain antibody is a NANOBODY®.

In certain embodiments, the split prime editor further comprises an affinity moiety that has affinity for either the DNA binding domain or the DNA polymerase domain. In some embodiments, the affinity moiety has affinity for the DNA binding domain. In some embodiments, the affinity moiety has affinity for the DNA polymerase domain. In some embodiments, the DNA binding domain comprises a peptide tag that is configured to bind to the affinity moiety and the DNA polymerase domain comprises the affinity moiety. In some embodiments, the DNA binding domain comprises the affinity moiety and the DNA polymerase domain comprises a peptide tag that is configured to bind to the affinity moiety. In some embodiments, the affinity moiety comprises an antibody or fragment thereof (e.g., a single domain antibody or a NANOBODY®). In some embodiments, the single-domain antibody comprises any one of the amino acid sequences as set forth in Table 17.

In some embodiments, the affinity moiety is fused to the first polypeptide and has affinity for the second amino acid sequence. In some embodiments, the affinity moiety is fused to the second polypeptide and has affinity for the first amino acid sequence. In some embodiments, the first polypeptide comprises a C-terminal intein sequence. In some embodiments, the second polypeptide comprises a N-terminal intein sequence. In some embodiments, assembly of the first polypeptide and the second polypeptide in a host cell results in fusion of the C-terminal intein sequence and the N-terminal intein sequence to generate a full intein sequence, which then results in splicing and excision of the full intein sequence. In certain embodiments, the first polypeptide comprises a first affinity moiety and the second polypeptide comprises a second affinity moiety. In some embodiments, the first affinity moiety has affinity for the second affinity moiety. In some embodiments, the first affinity moiety comprises a C-terminal leucine zipper monomer. In some embodiments, the second affinity moiety comprises an N-terminal leucine zipper monomer. In some embodiments, the C-terminal leucine zipper monomer and the N-terminal leucine zipper monomer forms a dimer in a host cell. In some embodiments, the first affinity moiety comprises a C-terminal dimerization domain. In some embodiments, the second affinity moiety comprises a N-terminal dimerization domain. In some embodiments, the C-terminal dimerization domain and the N-terminal dimerization domain form a dimer in a host cell.

In certain embodiments, the prime editor system comprises a scaffold RNA. In some embodiments, the first polypeptide and/or the second polypeptide comprises an adapter protein that has affinity for the scaffold RNA. Exemplary adapter proteins may include a MS2 coat/adapter protein (MCP), a PP7 adapter protein, a Qβ adapter protein, a F2 adapter protein, a GA adapter protein, a fr adapter protein, a JP501 adapter protein, a M12 adapter protein, a R17 adapter protein, a BZ13 adapter protein, a JP34 adapter protein, a JP500 adapter protein, a KU1 adapter protein, a M11 adapter protein, a MX1 adapter protein, a TW18 adapter protein, a VK adapter protein, a SP adapter protein, a FI adapter protein, a ID2 adapter protein, a NL95 adapter protein, a TW19 adapter protein, a AP205 adapter protein, a ϕCb5 adapter protein, a ϕCb8r adapter protein, a ϕ12r adapter protein, a ϕCb23r adapter protein, a 7s adapter protein and a PRR1 adapter protein.

In certain embodiments, the prime editor system further comprises a scaffold protein that has affinity for the first polypeptide and/or the second polypeptide. In some embodiments, the scaffold protein is fused to the first polypeptide or the second polypeptide. In some embodiments, the scaffold protein is not fused to either the first polypeptide or the second polypeptide. In some embodiments, the prime editor system further comprises a second scaffold protein that has affinity for the scaffold protein. In some embodiments, the second scaffold protein has affinity for the first polypeptide. In some embodiments, the second scaffold protein has affinity for to the second polypeptide. In some embodiments, the second scaffold protein is fused to the first polypeptide or the second polypeptide. In some embodiments, the second scaffold protein is not fused to either the first polypeptide or the second polypeptide.

In certain embodiments, the first polypeptide has affinity for an endogenous protein in a host cell. In some embodiments, the second polypeptide has affinity for the endogenous protein in a host cell.

In certain embodiments, the first polypeptide has affinity for a first endogenous protein in a host cell and the second polypeptide has affinity for a second endogenous protein in a host cell, and the first endogenous protein has affinity for the second endogenous protein.

In certain embodiments, the first polypeptide is configured to become covalently attached to the second polypeptide in a host cell. In some embodiments, the first polypeptide comprises a SpyTag peptide sequence and the second polypeptide comprises a SpyCatcher peptide sequence. In some embodiments, wherein the first polypeptide comprises a SnoopTag peptide sequence and the second polypeptide comprises a SnoopCatcher peptide sequence. In some embodiments, the first polypeptide comprises a SdyTag peptide sequence and the second polypeptide comprises a SdyCatcher peptide sequence. In some embodiments, the first polypeptide comprises a DogTag peptide sequence and the second polypeptide comprises a DogCatcher peptide sequence. In some embodiments, the first polypeptide comprises a SpyTag peptide sequence and the second polypeptide comprises a SpyDock peptide sequence. In some embodiments, the first polypeptide comprises an isopeptag peptide sequence and the second polypeptide comprises a Pilin-C peptide sequence.

In certain embodiments, the split prime editor comprises a third polypeptide encoding a third amino acid sequence. In some embodiments, the third amino acid sequence forms at least a portion of the DNA binding domain and/or the DNA polymerase domain.

In certain embodiments, the DNA binding domain comprises a CRISPR associated (Cas) protein domain. In some embodiments, the Cas protein domain is a Cas9. In some embodiments, the Cas9 comprises a mutation in an HNH domain. In some embodiments, the Cas protein domain has nickase activity. In some embodiments, the Cas9 comprises a H840A mutation in the HNH domain. In some embodiments, the Cas protein domain is a Cas12b. In some embodiments, the Cas protein domain is a Cas 12a, Cas12b, Cas12c, Cas12d, Cas12e, Cas14a, Cas14b, Cas14c, Cas14d, Cas14c, Cas 14f, Cas14g, Cas 14h, Cas 14u, or a Casφ. In some embodiments, the Cas protein domain comprises any one of the amino acid sequences as set forth in Table 14.

In some embodiments, the DNA polymerase domain comprises a reverse transcriptase. Many reverse transcriptase enzymes have DNA-dependent DNA synthesis abilities in addition to RNA-dependent DNA synthesis abilities, i.e., reverse transcription). In some embodiments, the reverse transcriptase is a retrovirus reverse transcriptase. In some embodiments, the reverse transcriptase is a Moloney murine leukemia virus (M-MLV) reverse transcriptase. In some embodiments, the reverse transcriptase comprises any one of the sequences as set forth in Table 11, Table 12, or Table 13.

In some embodiments provided herein, the first polypeptide and/or the second polypeptide comprises at least one peptide linker (e.g., at least two peptide linkers). In certain embodiments, the at least one peptide linker comprises 5 to 100 amino acids. In some embodiments, the at least one peptide linker comprises an amino acid sequence as set forth in Table 15.

In certain embodiments, the first polypeptide and/or the second polypeptide further comprises at least one nuclear localization sequence. In some embodiments, the at least one nuclear localization sequence comprises an amino acid sequence as set forth in Table 3.

In some embodiments, the first polypeptide and the second polypeptide are joined by a self-cleaving peptide. In some embodiments, the self-cleaving peptide is a P2A peptide (e.g., a P2A peptide comprising a sequence set forth in SEQ ID NO: 8004).

In certain embodiments, the prime editor comprises an amino acid sequence as set forth in Table 18. In certain embodiments, the prime editor comprises an amino acid sequence as set forth in Table 20 and/or Table 21. In certain embodiments, the first and/or second polypeptides comprise an amino acid sequence as set forth in Table 20. In certain embodiments, the first and/or second polypeptides comprise an amino acid sequence as set forth in Table 21.

In some aspects, provided herein is a split prime editing system comprising A) a first polypeptide, or a polynucleotide encoding the first polypeptide, the first polypeptide comprising a DNA binding domain fused to a first affinity moiety selected from: i) a single-domain antibody sequence, or ii) a peptide tag; and B) a second polypeptide, or a polynucleotide encoding the second polynucleotide, the second polynucleotide comprising a DNA polymerase domain fused to a second affinity moiety that is: i) the peptide tag if the DNA binding domain is fused to the single-domain antibody sequence, or ii) the single-domain antibody sequence if the DNA binding domain is fused to the peptide tag; wherein the peptide tag is an antigen for which the single-domain antibody sequence has sufficient affinity to bind under physiological conditions.

In some embodiments, the DNA binding domain comprises an HNH domain and/or a RuvC domain. In some embodiments, the DNA binding domain comprises both an HNH domain and a RuvC domain. In some embodiments, the DNA binding domain. In some embodiments, the DNA binding protein comprises a mutation that decreases or eliminates nuclease activity in the RuvC domain. The DNA binding domain may be a Type II Cas protein, such as a Cas9 protein. The Cas9 protein may be a Cas9 nickase. In some embodiments, the DNA binding domain is a Type V Cas protein. In other embodiments, the DNA binding domain is a Cas12 protein. In some embodiments, the DNA binding domain has a sequence with at least 80%, 85%, 90%, 95%, 96%, 97%, 98%, or 99% identity to a sequence from Table 14. In some embodiments, the DNA binding domain has a sequence from Table 14. In some embodiments, the sequence is a Cas9 nickase sequence from Table 8000.

In some embodiments, the DNA polymerase domain is a reverse transcriptase domain, such as a Maloney Murine Leukemia Virus (MMLV) reverse transcriptase. In some embodiments, the DNA polymerase domain comprises a sequence with at least 80%, 85%, 90%, 95%, 96%, 97%, 98%, or 99% identity to a sequence from Table 11, Table 12, or Table 13. In some embodiments, the DNA polymerase domain comprises a sequence from Table 11, Table 12, or Table 13.

In some embodiments, the DNA polymerase domain comprises a sequence having at least 80%, 85%, 90%, 95%, 96%, 97%, 98%, 99%, or 100% identity to SEQ ID NO: 4448 or SEQ ID NO: 8001.

In some embodiments, the single-domain antibody sequence has at least 80%, 85%, 90%, 95%, 96%, 97%, 98%, or 99% identity to SEQ ID NO: 8002. In some embodiments, the single-domain antibody sequence is SEQ ID NO: 8002.

In some embodiments, the peptide tag has a sequence from Table 16 or a sequence with 1 or 2 substitutions relative to a sequence from Table 16. In other embodiments, the peptide tag has a sequence from Table 16.

In some embodiments, the peptide tag is SEQ ID NO: 8003. In some embodiments, the DNA binding domain is located N-terminally to the first affinity moiety.

In some embodiments, the system further comprises a first peptide linker between the DNA binding domain and the first affinity moiety. In some embodiments, the first peptide linker comprises a sequence from Table 15. In some embodiments, the DNA polymerase domain is located C-terminally to the second affinity moiety. The system, as disclosed herein, may further comprise a second peptide linker between the DNA polymerase domain and the second affinity moiety (e.g., a second peptide linker comprising a sequence from Table 15).

In some embodiments, the first polypeptide further comprises one or more nuclear localization sequences (NLSs). The first polypeptide may comprise a C-terminal and an N-terminal NLS. The first polypeptide may further comprise a peptide linker between the N-terminal NLS and the DNA binding protein. In some embodiments, the peptide linker between the C-terminal NLS and the first binding moiety.

In some embodiments, the second polypeptide further comprises one or more nuclear localization sequences (NLSs). The second polypeptide may comprise a C-terminal and an N-terminal NLS. In some embodiments, a peptide linker is between the C-terminal NLS and the DNA polymerase domain. In some embodiments, a peptide linker between the N-terminal NLS and the second binding moiety. The NLS may have, individually, a sequence selected from Table 3 or a sequence having one or two substitutions relative to a sequence from Table 3.

In some embodiments, the peptide linkers have, individually, a sequence selected from Table 15 or a sequence having at least 80%, 85%, 90%, 95%, 96%, 97%, 98%, or 99% identity with a sequence from Table 15.

In some embodiments, the first polypeptide and the second polypeptide comprise compatible sequences from Table 21 or Table 20 or sequences having at least 80%, 85%, 90%, 95%, 96%, 97%, 98%, or 99% identity with compatible sequence from Table 21 or Table 20.

In some embodiments, the system further comprises a self-cleaving peptide joining the first polypeptide to the second polypeptide, such as a self-cleaving peptide comprising a sequence from Table 19 or a sequence having one or two substitutions relative to a sequence from Table 19. The self cleaving peptide may be a P2A peptide and comprise a sequence set forth in Table 19. In some embodiments, the self-cleaving peptide comprises SEQ ID NO: 8004.

In some embodiments, the system comprises a sequence having 80%, 85%, 90%, 95%, 96%, 97%, 98%, or 99% identity relative to a sequence from Table 18. In some embodiments, the system comprises a sequence selected from Table 18. In some embodiments, the sequence from Table 18 is SEQ ID NO: 8005 as set forth in Table 18.

In certain aspects, provided herein are lipid nanoparticles (LNPs) or ribonucleoproteins (RNPs) comprising a prime editing system described herein or a component thereof.

In certain aspects, provided herein are polynucleotides encoding a prime editor described herein. In some embodiments, the polynucleotide is operably linked to a regulatory element. In some embodiments, the regulatory element is an inducible regulatory element.

In certain aspects, provided herein are vectors (e.g., AAV vectors) comprising a polynucleotide described above.

In certain aspects, provided herein are polynucleotides encoding the first polypeptide described herein. In some embodiments, the polynucleotide is operably linked to a regulatory element. In some embodiments, the regulatory element is an inducible regulatory element.

In certain aspects, provided herein are vectors comprising a polynucleotide described above. In some embodiments, the vector is an AAV vector, such as a trans-splicing vector.

In certain aspects, provided herein are polynucleotides encoding the second polypeptide described herein. In some embodiments, the polynucleotide is operably linked to a regulatory element. In some embodiments, the regulatory element is an inducible regulatory element.

In certain aspects, provided herein are vectors comprising a polynucleotide described above. In some embodiments, the vector is an AAV vector trans-splicing vector.

In certain aspects, provided herein are kits comprising a first polynucleotide and a second polynucleotide, wherein the first polynucleotide is a polynucleotide described herein and the second polynucleotide is a polynucleotide described herein. In some embodiments, the first polynucleotide and/or the second polynucleotide is in a vector. In some embodiments, the vector is an AAV vector. In some embodiments, the vector is an AAV vector, such as trans-splicing vector.

In certain aspects, provided herein are isolated cells (e.g., human cells) comprising a prime editor system described herein, a LNP or RNP described herein, a polynucleotide described herein, or a vector described herein.

In certain aspects, provided herein are pharmaceutical compositions comprising i) a prime editor system described herein, a LNP or RNP described herein, a polynucleotide described herein, or a vector described herein; and (ii) a pharmaceutically acceptable carrier.

In certain embodiments, the prime editor systems described herein further comprise a prime editor guide RNA (a PEgRNA).

In certain aspects, provided herein are methods for editing a gene, the method comprising contacting the gene with a prime editor system described herein, wherein the PEgRNA directs the prime editor to incorporate the intended nucleotide edit in the gene, thereby editing the gene. In some embodiments, the prime editor synthesizes a single stranded DNA encoded by an editing template, wherein the single stranded DNA replaces an editing target sequence and results in incorporation of the intended nucleotide edit into a region corresponding to the editing target sequence in the gene. In some embodiments, the gene is in a cell (e.g., a mammalian cell (e.g., a human cell)). In some embodiments, the cell is in a subject (e.g., human).

In certain embodiments, the method further comprises administering the cell to a subject after incorporation of the intended nucleotide edit.

### BRIEF DESCRIPTION OF THE DRAWINGS

FIG. 1 is a schematic diagram showing an exemplary split prime editor. The split prime editor includes an spCas9, a Moloney Murine Leukemia Virus (MMLV) reverse transcriptase (RT), a Spot-Tag® (shown in uppercase, bold, and underlined), simian virus 40 (SV40) nuclear localization sequences (NLS) (shown in uppercase and italicized), a self-cleaving sequence P2A (shown in uppercase and underlined), a NANOBODY® sequence (shown in uppercase and bold), and intervening linkers (shown in lowercase). FIG. 1 discloses SEQ ID NOS 8703 and 8780-8781, respectively, in order of appearance.

FIG. 2 is a schematic diagram showing an exemplary split prime editor. The split prime editing system includes an spCas9, a Moloney Murine Leukemia Virus (MMLV) reverse transcriptase (RT), a Spot-Tag® (shown in uppercase, bold, and underlined), simian virus 40 (SV40) nuclear localization sequences (NLS) (shown in uppercase and italicized), a self-cleaving sequence P2A (shown in uppercase and underlined), a NANOBODY® sequence (shown in bold), and intervening linkers (shown in lowercase). FIG. 2 discloses SEQ ID NOS 8703, 8782 and 8781, respectively, in order of appearance.

FIG. 3 is a schematic diagram showing an exemplary split prime editor. The split prime editor includes an spCas9, a Moloney Murine Leukemia Virus (MMLV) reverse transcriptase (RT), also including a BC2 peptide (shown in uppercase, bold, and underlined), simian virus 40 (SV40) nuclear localization sequences (NLS) (shown in uppercase and italicized), a self-cleaving sequence P2A (shown in uppercase and underlined), a NANOBODY® sequence (shown in bold), and intervening linkers (shown in lowercase). FIG. 3 discloses SEQ ID NOS 8703, 8783 and 8781, respectively, in order of appearance.

FIG. 4 is a schematic diagram showing an exemplary split prime editor. The split prime editor includes an spCas9, a Moloney Murine Leukemia Virus (MMLV) reverse transcriptase (RT), and also includes a BC2 (shown in uppercase, bold, and underlined), simian virus 40 (SV40) nuclear localization sequences (NLS) (shown in uppercase and italicized), a self-cleaving sequence P2A (shown in uppercase and underlined), a NANOBODY® sequence (shown in bold), and intervening linkers (shown in lowercase). FIG. 4 discloses SEQ ID NOS 8703, 8784 and 8781, respectively, in order of appearance.

FIG. 5 is a graph showing percent editing of a target gene site (Fanconi anemia complementation group F (FANCF) gene site) by various exemplary configurations of the split prime editing systems. Gene editing activity for each of the split prime editing constructs (Cas9-BC2 NANOBODY®-MMLV, Cas9-NANOBODY® BC2-MMLV, Cas9-SpotTag® NANOBODY®-MMLV, and Cas9-NANOBODY® SpotTag®-MMLV) was compared to a control (fused) prime editor (PE2).

### DETAILED DESCRIPTION

Provided herein, in some embodiments, are compositions and methods related to split prime editors useful, for example, in prime editing applications. In certain embodiments, provided herein are compositions and methods for introducing intended nucleotide edits in target DNA, e.g., introducing a prime editing system comprising split prime editors. Compositions provided herein can comprise split prime editors comprising a DNA binding domain and a DNA polymerase domain (e.g., the split prime editor comprises a first polypeptide comprising a first amino acid sequence and a second polypeptide comprising a second amino acid sequence).

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. In some embodiments, the term primary cell means a cell isolated from an organism, e.g., a mammal, which is grown in tissue culture (i.e., in vitro) for the first time before subdivision and transfer to a subculture. In some non-limiting examples, mammalian primary cells can be modified through introduction of one or more polynucleotides, polypeptides, and/or prime editing compositions (e.g., through transfection, transduction, electroporation and the like) and further passaged. Such modified mammalian primary cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a fibroblast. In some embodiments, the cell is a stem cell. In some embodiments, the cell is a pluripotent stem cell. In some embodiments, the cell is an induced pluripotent stem cell (iPSC). In some embodiments, the cell is a stem cell. In some embodiments, the cell is an embryonic stem cell (ESC). In some embodiments, the cell is a human stem cell. In some embodiments, the cell is a human pluripotent stem cell. In some embodiments, the cell is a human fibroblast. In some embodiments, the cell is an induced human pluripotent stem cell (iPSC). In some embodiments, the cell is a human stem cell. In some embodiments, the cell is a human embryonic stem cell.

In some embodiments, a cell is not isolated from an organism but forms part of a tissue or organ of an organism, e.g., a mammal. In some non-limiting examples, mammalian cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a primary muscle cell. In some embodiments, the cell is a myosatellite cell (a satellite cell). In some embodiments, the cell is a human myosatellite cell (a satellite cell). In some embodiments, the cell is a stem cell. In some embodiments, the cell is a human stem cell.

In some embodiments, the cell is a differentiated cell. In some embodiments, cell is a fibroblast. In some embodiments, the cell is a differentiated muscle cell, a myosatellite cell, a differentiated epithelial cell, or a differentiated neuron cell. In some embodiments, the cell is a skeletal muscle cell. In some embodiments, the skeletal muscle cell is differentiated from an iPSC, ESC or myosatellite cell. In some embodiments, the cell is a differentiated human cell. In some embodiments, cell is a human fibroblast. In some embodiments, the cell is a differentiated human muscle cell. In some embodiments, cell is a human myosatellite cell. In some embodiments, the cell is a human skeletal muscle cell. In some embodiments, the human skeletal muscle cell is differentiated from a human iPSC, human ESC or human myosatellite cell. In some embodiments, the cell is differentiated from a human iPSC or human ESC.

In some embodiments, the cell comprises a prime editor (e.g., a split prime editor), a PEgRNA, a ngRNA, a prime editing system, or a prime editing complex. In some embodiments, the cell is from a human subject. In some embodiments, the human subject has a disease or condition associated with a mutation to be corrected by prime editing. In some embodiments, the cell is from a human subject, and comprises a prime editor (e.g., a split prime editor), a PERNA, a ngRNA, a prime editing system, or a prime editing complex for correction of the mutation. In some embodiments, the cell is from the human subject and the mutation has been edited or corrected by prime editing. In some embodiments, the cell is in a human subject, and comprises a prime editor (e.g., a split prime editor), a PEgRNA, a ngRNA, a prime editing system, or a prime editing complex for correction of the mutation. In some embodiments, the cell is from the human subject and the mutation has been edited or corrected by prime editing.

As used herein, “intein” refers an auto-catalytic protein segments capable of excising itself from a larger precursor protein, enabling the flanking extein (external protein) sequences to be ligated through the formation of a new peptide bond (e.g., protein splicing). Inteins may include a protein domain sequence that can spontaneously splice (e.g., splice from protein flanking N- and C-terminal domains) and excise itself from a sequence to become a mature protein.

As used herein, “leucine zipper” refers to an amphipathic a helix containing heptad repeats of Leu residues on one face of the helix and serves as a dimerization module. On dimerization, the leucine-zipper a helices form a parallel-coiled coil based on hydrophobic interfacial side-chain packing. The dimerization brings a molecular surface (e.g., a DNA-binding surface) to the positions appropriate for contacting the surface in a scissor-grip mode or in an induced helical fork mode. A leucine zipper motif is commonly motif found in many DNA-binding proteins, including transcription factors such as C/EBP, Jun, Fos, GCN4, and HSF.

As used herein, “passively assemble” or “passive assembly” refers to a process in which an organized structure forms from individual components, as a result of specific, local interactions among the individual components, without the aid of external components (e.g., two or more split prime editor fragments or sequences associate inside a cell to reconstitute a split prime editor without aid of additional peptides).

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a split prime editor may be a protein comprising a Cas9 protein domain of S. pyogenes and a reverse transcriptase protein domain of Moloney murine leukemia virus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9, but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose. In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, primer binding site or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16:276-277), and the GGSEARCH program https://fasta.bioch.virginia.edu/fasta_www2/, which is part of the FASTA package (Pearson W and Lipman D, 1988, Proc. Natl. Acad. Sci. USA, 85:2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a thymine or uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion of two polynucleotide molecules. In some embodiments, the portion of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., an mRNA or coding RNA, is determined by the amount of a functional form of the protein encoded by the polypeptide after translation of the polynucleotide.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLID sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be up to about 100 years of age. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen.

In some embodiments, the antibody may be a single domain antibody (e.g., a NANOBODY®). In some embodiments, the single domain antibody is a recombinant variable domain of a heavy-chain-only antibody. For example, a single domain antibody can include a VHH, a humanized VHH or a camelized VH (such as a camelized human VH) or generally a sequence optimized VHH (such as e.g., optimized for chemical stability and/or solubility, maximum overlap with known human framework regions and maximum expression).

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” may refer to a quantity of a composition, for example a composition comprising a construct, that can be sufficient to result in a desired activity upon introduction into a subject as disclosed herein. An effective amount of the prime editing compositions can be provided to the target gene or cell, whether the cell is ex vivo or in vivo.

An effective amount can be the amount to induce, for example, at least about a 2-fold change (increase or decrease) or more in the amount of target nucleic acid modulation (e.g., expression of a gene to produce functional a protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target gene to produce a functional protein).

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

### Prime Editing

The term “prime editing” refers to programmable editing of a target DNA using a prime editor complexed with a PEgRNA to incorporate an intended nucleotide sequence modification into the target DNA through target-primed DNA synthesis. A target polynucleotide (e.g., a target gene) of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a protospacer adjacent motif (PAM) sequence. In prime editing using a Cas-protein-based split prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is 3 base pairs upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 base pairs upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

In some embodiments, a PEgRNA complexes with and directs a split prime editor to bind to the search target sequence of the target gene. In some embodiments, the bound split prime editor generates a nick on the edit strand (PAM strand) of the target gene at the nick site. In some embodiments, a primer binding site (PBS) of the PEgRNA anneals with a free 3′ end formed at the nick site, and the split prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to the endogenous target gene sequence. In some embodiments, the editing template of a PEgRNA is complementary to a sequence in the edit strand except for one or more mismatches at the intended nucleotide edit positions in the editing template partially complementary to the editing template may be referred to as an “editing target sequence”. Accordingly, in some embodiments, the newly synthesized single stranded DNA has identity or substantial identity to a sequence in the editing target sequence, except for one or more insertions, deletions, or substitutions at the intended nucleotide edit positions.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the split prime editor, either linked to other components of the split prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Split Prime Editors

The term “split prime editor (PE)” refers to a prime editor composed of at least two polypeptides (e.g., a first polypeptide and a second polypeptide) that individually are not capable of functioning as a prime editor but that are able to associate under physiological conditions to facilitate prime editing. Advantageously, the individual polypeptides of the split prime editor (or nucleic acids encoding the individual polypeptides of the split prime editor) can be separately delivered to a cell where they associate to form a split prime editor and mediate prime editing. Split prime editors can therefore, for example, be delivered to cells using delivery systems having a smaller payload capacity than a corresponding intact prime editor. As used herein, a split prime editor includes, but is not limited to, protein constructs wherein the first polypeptide and the second polypeptide are joined by a self-cleaving peptide. Therefore, the split prime editor includes embodiments where the split prime editor is a single polypeptide configured to produce at least two polypeptides prior to prime editing.

In some embodiments, the split prime editor comprises a DNA binding domain and a DNA polymerase domain, wherein the split prime editor comprises a first polypeptide comprising a first amino acid sequence and a second polypeptide comprising a second amino acid sequence.

In certain embodiments, the first amino acid sequence forms at least a portion of the DNA binding domain, and the second amino acid sequence forms at least a portion of the DNA polymerase domain. In some embodiments, the first amino acid sequence forms the entirety of the DNA binding domain and the second amino acid sequence forms the entirety of the DNA polymerase domain. In some embodiments, the first amino acid sequence forms the entirety of the DNA binding domain and a portion of the DNA polymerase domain, while the second amino acid sequence forms a portion of the DNA polymerase domain. In some embodiments, the first amino acid sequence forms a portion of the DNA binding domain and the second amino acid sequence form a portion of the DNA binding domain and the entirety of the DNA polymerase domain.

In certain embodiments, the first amino acid sequence forms at least a portion of the DNA polymerase domain, and the second amino acid sequence forms at least a portion of the DNA binding domain. In some embodiments, the first amino acid sequence forms the entirety of the DNA polymerase domain and the second amino acid sequence forms the entirety of the DNA binding domain. In some embodiments, the second amino acid sequence forms the entirety of the DNA binding domain and a portion of the DNA polymerase domain. In some embodiments, the first amino acid sequence forms the entirety of the DNA polymerase domain and a portion of the DNA binding domain, while the second amino acid sequence forms a portion of the DNA binding domain. In some embodiments, the first amino acid sequence forms a portion of the DNA polymerase domain and the second amino acid sequence form a portion of the DNA polymerase domain and the entirety of the DNA binding domain.

In various embodiments, a split prime editor includes a polypeptide domain having DNA binding activity and a polypeptide domain having DNA polymerase activity.

In some embodiments, the split prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the split prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the split prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation.

A split prime editor may be engineered. In some embodiments, the polypeptide components of a split prime editor do not naturally occur in the same organism or cellular environment. In some embodiments, the polypeptide components of a split prime editor may be of different origins or from different organisms. In some embodiments, a split prime editor comprises a DNA binding domain and a DNA polymerase domain that are derived from different species. In some embodiments, a split prime editor comprises a Cas polypeptide and a reverse transcriptase polypeptide that are derived from different species. For example, a split prime editor may comprise a S. pyogenes Cas9 polypeptide and a Moloney murine leukemia virus (M-MLV) reverse transcriptase polypeptide.

In some embodiments, a split prime editor comprises one or more polypeptide domains provided in trans as separate proteins, which are capable of being associated to each other, for example, through non-peptide linkages or through aptamers or recruitment sequences. A split prime editor may comprise a DNA binding domain and a reverse transcriptase domain associated with each other by an RNA-protein recruitment aptamer, e.g., a MS2 aptamer/adapter protein, which may be linked to a PEgRNA. Prime editor polypeptide components may be encoded by one or more polynucleotides in whole or in part. In some embodiments, a single polynucleotide, construct, or vector encodes the split prime editor. In some embodiments, multiple polynucleotides, constructs, or vectors each encode a polypeptide domain or portion of a domain of a split prime editor, or a portion of a split prime editor. For example, a split prime editor may comprise an N-terminal portion fused to an intein-N and a C-terminal portion fused to an intein-C, each of which is individually encoded by an AAV vector.

A split prime editor may comprise two polypeptides that are capable of associating with each other via the interactions of a single-domain antibody fused to one of the polypeptides and a peptide tag or antigen fused to the second polypeptide. In some embodiments, the two polypeptides are fused via a self-cleaving peptide. In other embodiments, the two polypeptide domains are provided in trans. In some embodiments, a first polypeptide comprises a DNA binding domain fused to a single-domain antibody and the second polypeptide comprises a DNA polymerase domain fused to a peptide tag. In other embodiments, the first polypeptide comprises a DNA binding domain fused to a peptide tag and the second polypeptide comprises a DNA polymerase domain fused to a single-domain antibody. In any embodiment, the first and second polypeptide can further comprise one or more nuclear localization sequences (NLSs). For example, the first polypeptide can comprise an NLS located N-terminally to the DNA biding domain, an NLS located C-terminally to the DNA binding domain, or both; and the second polypeptide can comprise an NLS located N-terminally to the DNA polymerase domain, an NLS located C-terminally to the DNA polymerase domain, or both. Peptide linkers can optionally be included between any of the individual components of a polypeptide.

Suitable DNA binding domains include, but are not limited to, any Cas protein or variant (e.g., a type II or type IV Cas protein). Exemplary Cas proteins and variants can be found in Tables 1 and 2. The Cas protein can be any Cas protein comprising a RuvC domain, an HNH domain, or both. The Cas protein can be a nickase or a nuclease active Cas protein. Suitable sequences DNA binding domain include, but are not limited to, any sequence found in Table 14; or any sequence having at least 80%, 85%, 90%, 95%, 96%, 97%, 98%, or 99% identity with a sequence found in Table 14.

Suitable DNA polymerase domains include, but are not limited to, reverse transcriptase domains. Such DNA polymerase domains include, but are not limited to, any sequence found in Table 11, Table 12, or Table 13; or any sequence having at least 80%, 85%, 90%, 95%, 96%, 97%, 98%, or 99% identity with a sequence found in Table 11, Table 12, or Table 13.

Suitable peptide tag sequences include, but are not limited to, sequences found in Table 16, including sequences that have one or two substitutions compared to a sequence in Table 16. Suitable single domain antibody sequences include, but are not limited to, sequences found in Table 17, including sequences having at least 80%, 85%, 90%, 95%, 96%, 97%, 98%, or 99% identity to a sequence in Table 17. Any of the peptide tag sequences in Table 16 can be paired with a single-domain antibody sequence of Table 17 in a split prime editor system.

Suitable NLS sequences include, but are not limited to, any sequence found in Table 3, or a sequence having one or two substitutions compared to a sequence found in Table 3.

Suitable linker peptide sequences include, but are not limited to, any sequence found in Table 15, or a sequence having at least 80%, 85%, 90%, 95%, 96%, 97%, 98%, or 99% identity to a sequence in Table 15.

Suitable self-cleaving peptide sequences include, but are not limited to, any sequence found in Table 19, or a sequence having at least 80%, 85%, 90%, 95%, 96%, 97%, 98%, or 99% identity to a sequence in Table 19.

In some embodiments, the split prime editor comprises two peptides not joined by a self-cleaving peptide. In certain embodiments, the prime editor comprises an amino acid sequence as set forth in Table 20 and/or Table 21.

In some embodiments, the first polypeptide comprises, from N-terminus to C-terminus, a DNA binding domain, a first peptide linker, a peptide tag, a second peptide linker, and a nuclear localization sequence (NLS). In some embodiments, the first polypeptide may further comprise a second NLS located N-terminally of the DNA binding domain. In such embodiments, the second NLS may be attached to the DNA binding domain via a third peptide linker. In some embodiments, the second polypeptide comprises, from N-terminus to C-terminus, an NLS, an optional first peptide linker, a single-domain antibody amino acid sequence, a second peptide linker, and a DNA polymerase domain. In some embodiments, the second polypeptide may further comprise a second NLS located C-terminally of the DNA polymerase domain. In such embodiments, the second NLS may be attached to the DNA polymerase via a third peptide linker. Exemplary first and second polypeptide sequences can be found in Table 20.

In some embodiments, the first polypeptide comprises, from N-terminus to C-terminus, a DNA binding domain, a first peptide linker, a single-domain antibody amino acid sequence, an optional second peptide linker, and an NLS. In some embodiments, the first polypeptide may further comprise a second NLS located N-terminally of the DNA binding domain. In such embodiments, the second NLS may be attached to the DNA binding domain via a third peptide linker. In some embodiments, the second polypeptide comprises, from N-terminus to C-terminus, an NLS, a first peptide linker, a peptide tag, a second peptide linker, and a DNA polymerase domain. In some embodiments, the second polypeptide may further comprise a second NLS located C-terminally of the DNA polymerase domain. In such embodiments, the second NLS may be attached to the DNA polymerase via a third peptide linker. Exemplary first and second polypeptide sequences can be found in Table 21.

In some embodiments, the first polypeptide comprises, from N-terminus to C-terminus, a DNA binding domain, a first peptide linker, an NLS, an optional second peptide linker, and a single-domain antibody amino acid sequence. In some embodiments, the first peptide may further comprise a second NLS located N-terminally of the DNA binding domain. In such embodiments, the second NLS may be attached to the DNA binding domain via a third peptide linker. In some embodiments, the second polypeptide comprises, from N-terminus to C-terminus, a peptide tag, a first peptide linker, an NLS, a second peptide linker, and a DNA polymerase domain. In some embodiments, the second peptide may further comprise a second NLS located C-terminally of the DNA polymerase domain. In such embodiments, the second NLS may be attached to the DNA polymerase domain via a third peptide linker.

In some embodiments, the first polypeptide comprises, from N-terminus to C-terminus, a DNA binding domain, a first peptide linker, an NLS, a second peptide linker, and a peptide tag. In some embodiments, the first polypeptide may further comprise a second NLS located N-terminally of the DNA binding domain. In such embodiments, the second NLS may be connected to the DNA binding domain via a third peptide linker. In some embodiments, the second polypeptide comprises, from N-terminus to C-terminus, a single-domain antibody amino acid sequence, an optional first peptide linker, an NLS, a second peptide linker, and a DNA polymerase domain. In some embodiments, the second polypeptide further comprises a second NLS located C-terminally of the DNA polymerase domain. In such embodiments, the second NLS may be attached to the DNA polymerase domain via a third peptide linker.

In some embodiments, the split prime editor comprises, from N-terminus to the C-terminus, a DNA binding domain, a first peptide linker, a peptide tag, a second peptide linker, a first nuclear localization sequence (NLS), a self-cleaving peptide, a second NLS, an optional third peptide linker, a single-domain antibody amino acid sequence, a fourth peptide linker, and a DNA polymerase domain. In some embodiments, the split prime editor further comprises a third NLS located N-terminally of the DNA binding domain. In such embodiments, the third NLS may be attached to the DNA binding domain via a fifth peptide linker. In some embodiments, the split prime editor further comprises a fourth NLS located C-terminally of the DNA polymerase domain. In such embodiments, the fourth NLS may be attached to the DNA polymerase domain via a sixth peptide linker.

In some embodiments, the split prime editor comprises, from N-terminus to the C-terminus, a DNA binding domain, a first peptide linker, a single-domain antibody amino acid sequence, an optional second linker, a first NLS, a self-cleaving peptide, a second NLS, a third peptide linker, a peptide tag, a fourth peptide linker, and a DNA polymerase domain. In some embodiments, the split prime editor further comprises a third NLS located N-terminally of the DNA binding domain. In such embodiments, the third NLS may be attached to the DNA binding domain via a fifth peptide linker. In some embodiments, the split prime editor further comprises a fourth NLS located C-terminally of the DNA polymerase domain. In such embodiments, the fourth NLS may be attached to the DNA polymerase domain via a sixth peptide linker.

In some embodiments, the split prime editor comprises, from N-terminus to the C-terminus, a DNA binding domain, a first peptide linker, a single-domain antibody amino acid sequence, an optional second peptide linker, a first NLS, a self-cleaving peptide, a second NLS, a third peptide linker, a peptide tag, a fourth peptide linker, and a DNA polymerase domain. In some embodiments, the split prime editor further comprises a third NLS located N-terminally of the DNA binding domain. In such embodiments, the third NLS may be attached to the DNA binding domain via a fifth peptide linker. In some embodiments, the split prime editor further comprises a fourth NLS located C-terminally of the DNA polymerase domain. In such embodiments, the fourth NLS may be attached to the DNA polymerase domain via a sixth peptide linker.

In some embodiments, the split prime editor comprises, from N-terminus to the C-terminus, a DNA binding domain, a first peptide linker, a peptide tag, a second peptide linker, a first NLS, a self-cleaving peptide, a second NLS, an optional third peptide linker, a single-domain antibody amino acid sequence, a fourth peptide linker, and a DNA polymerase domain. In some embodiments, the split prime editor further comprises a third NLS located N-terminally of the DNA binding domain. In such embodiments, the third NLS may be attached to the DNA binding domain via a fifth peptide linker. In some embodiments, the split prime editor further comprises a fourth NLS located C-terminally of the DNA polymerase domain. In such embodiments, the fourth NLS may be attached to the DNA polymerase domain via a sixth peptide linker.

In some embodiments, the split prime editor system comprises a self-cleaving peptide linker between the first and second polypeptides and has an amino acid sequence as set forth in Table 18.

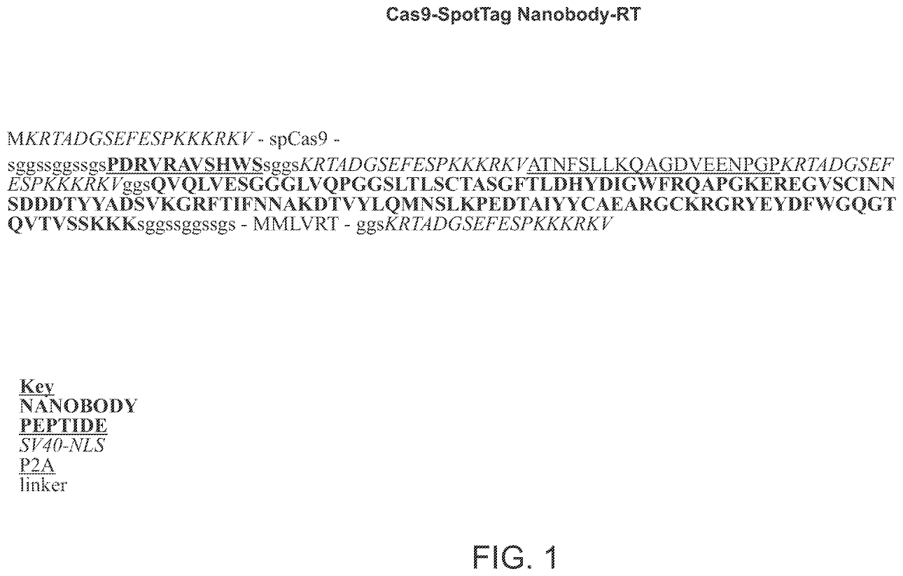

In some embodiments, the split prime editor comprises, from the N-terminus to the C-terminus, a first nuclear localization sequence (NLS), an spCas9 amino acid sequence, a first peptide linker, a SpotTag® peptide tag, a second peptide linker, a second NLS, a self-cleaving peptide, a third NLS, a third peptide linker, a single-domain antibody amino acid sequence, a fourth peptide linker, a reverse transcriptase amino acid sequence, a fifth peptide linker, and a fourth NLS (as shown in FIG. 1 and in Table 18).

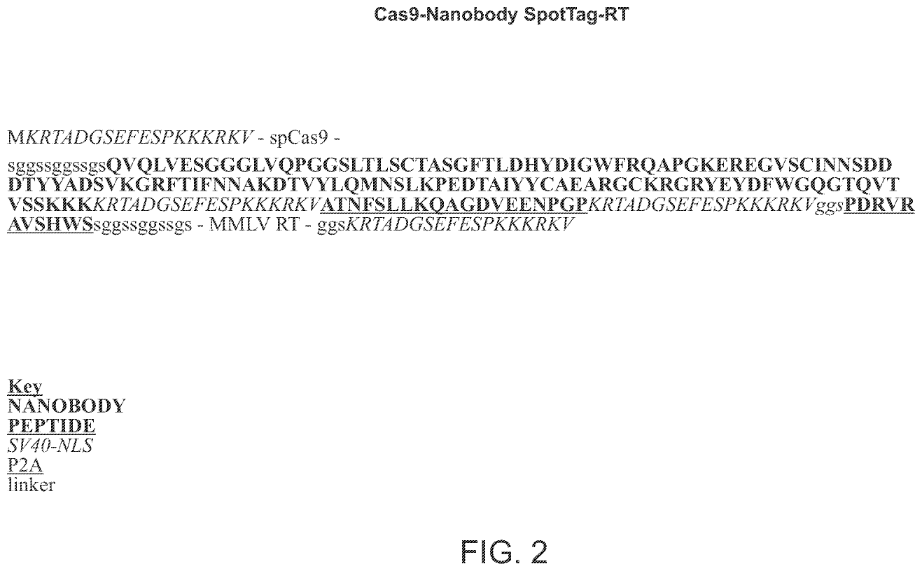

In some embodiments, the split prime editor comprises, from the N-terminus to the C-terminus, a first NLS, an spCas9 amino acid sequence, a first peptide linker, a single-domain antibody amino acid sequence, a second NLS, a self-cleaving peptide, a third NLS, a second peptide linker, a SpotTag® peptide tag, a third peptide linker, a reverse transcriptase amino acid sequence, a fourth peptide linker, and a fourth NLS (as shown in FIG. 2 and in Table 18).

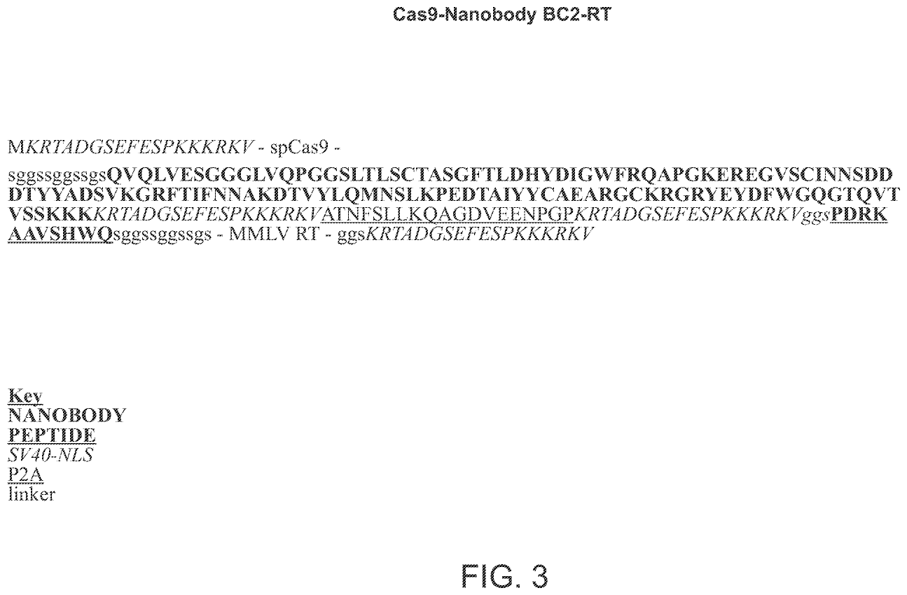

In some embodiments, the split prime editor comprises, from the N-terminus to the C-terminus, a first NLS, an spCas9 amino acid sequence, a first peptide linker, a single-domain antibody amino acid sequence, a second NLS, a self-cleaving peptide, a third NLS, a second peptide linker, a BC2 peptide tag, a third peptide linker, a reverse transcriptase amino acid sequence, a fourth peptide linker, and a fourth NLS (as shown in FIG. 3 and in Table 18).

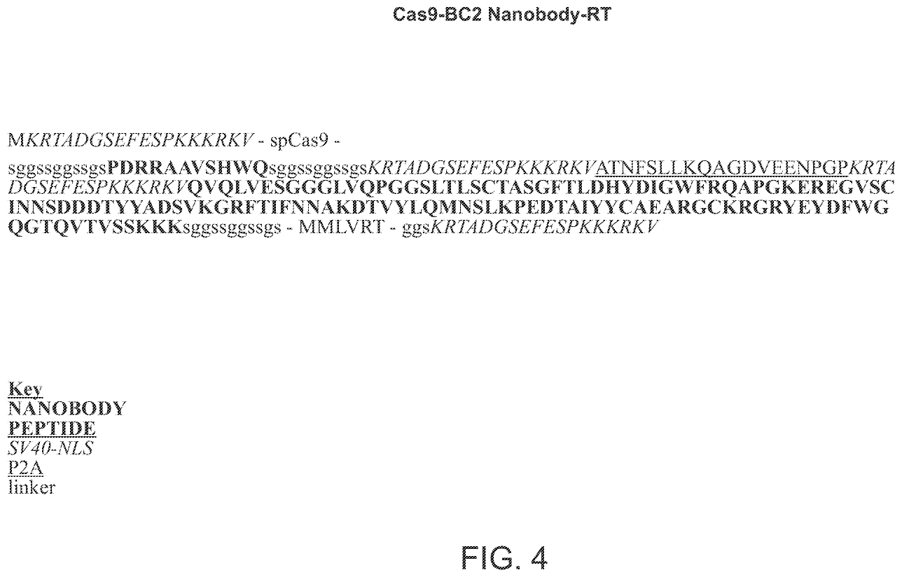

In some embodiments, the split prime editor comprises, from the N-terminus to the C-terminus, a first NLS, an spCas9 amino acid sequence, a first peptide linker, a BC2 peptide tag, a second peptide linker, a second NLS, a self-cleaving peptide, a third NLS, a single-domain antibody amino acid sequence, a third peptide linker, a reverse transcriptase amino acid sequence, a fourth peptide linker, and a fourth NLS (as shown in FIG. 4 and in Table 18).

TABLE 18
Amino acid sequences of exemplary self-cleaving peptide split
prime editor systems

| SEQ ID NO: | Split prime editor configuration |
|---|---|
| 8005 | MKRTADGSEFESPKKKRKVDKKYSIGLDIGTNSVGWAVITDEYKVPSK |
| | KFKVLGNTDRHSIKKNLIGALLFDSGETAEATRLKRTARRRYTRRKNR |
| | ICYLQEIFSNEMAKVDDSFFHRLEESFLVEEDKKHERHPIFGNIVDEVA |
| | YHEKYPTIYHLRKKLVDSTDKADLRLIYLALAHMIKFRGHFLIEGDLNP |
| | DNSDVDKLFIQLVQTYNQLFEENPINASGVDAKAILSARLSKSRRLENL |
| | IAQLPGEKKNGLFGNLIALSLGLTPNFKSNFDLAEDAKLQLSKDTYDD |
| | DLDNLLAQIGDQYADLFLAAKNLSDAILLSDILRVNTEITKAPLSASMI |
| | KRYDEHHQDLTLLKALVRQQLPEKYKEIFFDQSKNGYAGYIDGGASQ |
| | EEFYKFIKPILEKMDGTEELLVKLNREDLLRKQRTFDNGSIPHQIHLGEL |
| | HAILRRQEDFYPFLKDNREKIEKILTFRIPYYVGPLARGNSRFAWMTRK |
| | SEETITPWNFEEVVDKGASAQSFIERMTNFDKNLPNEKVLPKHSLLYE |
| | YFTVYNELTKVKYVTEGMRKPAFLSGEQKKAIVDLLFKTNRKVTVKQ |
| | LKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWG |
| | RLSRKLINGIRDKQSGKTILDFLKSDGFANRNFMQLIHDDSLTFKEDIQ |
| | KAQVSGQGDSLHEHIANLAGSPAIKKGILQTVKVVDELVKVMGRHKP |
| | ENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSI |
| | DNKVLTRSDKNRGKSDNVPSEEVVKKMKNYWRQLLNAKLITQRKFD |
| | NLTKAERGGLSELDKAGFIKRQLVETRQITKHVAQILDSRMNTKYDEN |
| | DKLIREVKVITLKSKLVSDFRKDFQFYKVREINNYHHAHDAYLNAVV |
| | GTALIKKYPKLESEFVYGDYKVYDVRKMIAKSEQEIGKATAKYFFYSN |
| | IMNFFKTEITLANGEIRKRPLIETNGETGEIVWDKGRDFATVRKVLSMP |
| | QVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKG |
| | YKEVKKDLIIKLPKYSLFELENGRKRMLASAGELQKGNELALPSKYVN |
| | FLYLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTTID |
| | RKRYTSTKEVLDATLIHQSITGLYETRIDLSQLGGDSGGSSGGSSGSPD |
| | RVRAVSHWSSGGSKRTADGSEFESPKKKRKVATNFSLLKQAGDVEEN |
| | PGPKRTADGSEFESPKKKRKVGGSQVQLVESGGGLVQPGGSLTLSCTA |
| | SGFTLDHYDIGWFRQAPGKEREGVSCINNSDDDTYYADSVKGRFTIFN |
| | NAKDTVYLQMNSLKPEDTAIYYCAEARGCKRGRYEYDFWGQGTQVT |
| | VSSKKKNIEDEYRLHETSKEPDVSLGSTWLSDFPQAWAETGGMGLAV |
| | RQAPLIIPLKATSTPVSIKQYPMSQEARLGIKPHIQRLLDQGILVPCQSP |
| | WNTPLLPVKKPGTNDYRPVQDLREVNKRVEDIHPTVPNPYNLLSGLPP |
| | SHQWYTVLDLKDAFFCLRLHPTSQPLFAFEWRDPEMGISGQLTWTRLP |
| | QGFKNSPTLFNEALHRDLADFRIQHPDLILLQYVDDLLLAATSELDCQ |
| | QGTRALLQTLGNLGYRASAKKAQICQKQVKYLGYLLKEGQRWLTEA |
| | RKETVMGQPTPKTPRQLREFLGKAGFCRLFIPGFAEMAAPLYPLTKPG |
| | TLFNWGPDQQKAYQEIKQALLTAPALGLPDLTKPFELFVDEKQGYAK |
| | GVLTQKLGPWRRPVAYLSKKLDPVAAGWPPCLRMVAAIAVLTKDAG |
| | KLTMGQPLVILAPHAVEALVKQPPDRWLSNARMTHYQALLLDTDRV |
| | QFGPVVALNPATLLPLPEEGLQHNCLDILAEAHGTRPDLTDQPLPDAD |
| | HTWYTDGSSLLQEGQRKAGAAVTTETEVIWAKALPAGTSAQRAELIA |
| | LTQALKMAEGKKLNVYTDSRYAFATAHIHGEIYRRRGWLTSEGKEIK |
| | NKDEILALLKALFLPKRLSIIHCPGHQKGHSAEARGNRMADQAARKAA |
| | ITETPDTSTLLIENSSPSGGSKRTADGSEFEPKKKRKV- |
| 8006 | MKRTADGSEFESPKKKRKVDKKYSIGLDIGTNSVGWAVITDEYKVPSK |
| | KFKVLGNTDRHSIKKNLIGALLFDSGETAEATRLKRTARRRYTRRKNR |
| | ICYLQEIFSNEMAKVDDSFFHRLEESFLVEEDKKHERHPIFGNIVDEVA |
| | YHEKYPTIYHLRKKLVDSTDKADLRLIYLALAHMIKFRGHFLIEGDLNP |
| | DNSDVDKLFIQLVQTYNQLFEENPINASGVDAKAILSARLSKSRRLENL |
| | IAQLPGEKKNGLFGNLIALSLGLTPNFKSNFDLAEDAKLQLSKDTYDD |
| | DLDNLLAQIGDQYADLFLAAKNLSDAILLSDILRVNTEITKAPLSASMI |
| | KRYDEHHQDLTLLKALVRQQLPEKYKEIFFDQSKNGYAGYIDGGASQ |
| | EEFYKFIKPILEKMDGTEELLVKLNREDLLRKQRTFDNGSIPHQIHLGEL |
| | HAILRRQEDFYPFLKDNREKIEKILTFRIPYYVGPLARGNSRFAWMTRK |
| | SEETITPWNFEEVVDKGASAQSFIERMTNFDKNLPNEKVLPKHSLLYE |
| | YFTVYNELTKVKYVTEGMRKPAFLSGEQKKAIVDLLFKTNRKVTVKQ |
| | LKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWG |
| | RLSRKLINGIRDKQSGKTILDFLKSDGFANRNFMQLIHDDSLTFKEDIQ |
| | KAQVSGQGDSLHEHIANLAGSPAIKKGILQTVKVVDELVKVMGRHKP |
| | ENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSI |
| | DNKVLTRSDKNRGKSDNVPSEEVVKKMKNYWRQLLNAKLITQRKFD |
| | NLTKAERGGLSELDKAGFIKRQLVETRQITKHVAQILDSRMNTKYDEN |
| | DKLIREVKVITLKSKLVSDFRKDFQFYKVREINNYHHAHDAYLNAVV |
| | GTALIKKYPKLESEFVYGDYKVYDVRKMIAKSEQEIGKATAKYFFYSN |
| | IMNFFKTEITLANGEIRKRPLIETNGETGEIVWDKGRDFATVRKVLSMP |
| | QVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKG |
| | YKEVKKDLIIKLPKYSLFELENGRKRMLASAGELQKGNELALPSKYVN |
| | FLYLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTTID |
| | RKRYTSTKEVLDATLIHQSITGLYETRIDLSQLGGDSGGSSGGSSGSQV |
| | QLVESGGGLVQPGGSLTLSCTASGFTLDHYDIGWFRQAPGKEREGVSC |
| | INNSDDDTYYADSVKGRFTIFNNAKDTVYLQMNSLKPEDTAIYYCAEA |
| | RGCKRGRYEYDFWGQGTQVTVSSKKKKRTADGSEFESPKKKRKVAT |
| | NFSLLKQAGDVEENPGPKRTADGSEFESPKKKRKVGGSPDRVRAVSH |
| | WSSGGSSGGSSGSNIEDEYRLHETSKEPDVSLGSTWLSDFPQAWAETG |
| | GMGLAVRQAPLIIPLKATSTPVSIKQYPMSQEARLGIKPHIQRLLDQGIL |
| | VPCQSPWNTPLLPVKKPGTNDYRPVQDLREVNKRVEDIHPTVPNPYNL |
| | LSGLPPSHQWYTVLDLKDAFFCLRLHPTSQPLFAFEWRDPEMGISGQL |
| | TWTRLPQGFKNSPTLFNEALHRDLADFRIQHPDLILLQYVDDLLLAATS |
| | ELDCQQGTRALLQTLGNLGYRASAKKAQICQKQVKYLGYLLKEGQR |
| | WLTEARKETVMGQPTPKTPRQLREFLGKAGFCRLFIPGFAEMAAPLYP |
| | LTKPGTLFNWGPDQQKAYQEIKQALLTAPALGLPDLTKPFELFVDEKQ |
| | GYAKGVLTQKLGPWRRPVAYLSKKLDPVAAGWPPCLRMVAAIAVLT |
| | KDAGKLTMGQPLVILAPHAVEALVKQPPDRWLSNARMTHYQALLLD |
| | TDRVQFGPVVALNPATLLPLPEEGLOHNCLDILAEAHGTRPDLTDQPL |
| | PDADHTWYTDGSSLLQEGQRKAGAAVTTETEVIWAKALPAGTSAQR |
| | AELIALTQALKMAEGKKLNVYTDSRYAFATAHIHGEIYRRRGWLTSE |
| | GKEIKNKDEILALLKALFLPKRLSIIHCPGHQKGHSAEARGNRMADQA |
| | ARKAAITETPDTSTLLIENSSPSGGSKRTADGSEFEPKKKRKV- |
| 8007 | MKRTADGSEFESPKKKRKVDKKYSIGLDIGTNSVGWAVITDEYKVPSK |
| | KFKVLGNTDRHSIKKNLIGALLFDSGETAEATRLKRTARRRYTRRKNR |
| | ICYLQEIFSNEMAKVDDSFFHRLEESFLVEEDKKHERHPIFGNIVDEVA |
| | YHEKYPTIYHLRKKLVDSTDKADLRLIYLALAHMIKFRGHFLIEGDLNP |
| | DNSDVDKLFIQLVQTYNQLFEENPINASGVDAKAILSARLSKSRRLENL |
| | IAQLPGEKKNGLFGNLIALSLGLTPNFKSNFDLAEDAKLQLSKDTYDD |
| | DLDNLLAQIGDQYADLFLAAKNLSDAILLSDILRVNTEITKAPLSASMI |
| | KRYDEHHQDLTLLKALVRQQLPEKYKEIFFDQSKNGYAGYIDGGASQ |
| | EEFYKFIKPILEKMDGTEELLVKLNREDLLRKQRTFDNGSIPHQIHLGEL |
| | HAILRRQEDFYPFLKDNREKIEKILTFRIPYYVGPLARGNSRFAWMTRK |
| | SEETITPWNFEEVVDKGASAQSFIERMTNFDKNLPNEKVLPKHSLLYE |
| | YFTVYNELTKVKYVTEGMRKPAFLSGEQKKAIVDLLFKTNRKVTVKQ |
| | LKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWG |
| | RLSRKLINGIRDKQSGKTILDFLKSDGFANRNFMQLIHDDSLTFKEDIQ |
| | KAQVSGQGDSLHEHIANLAGSPAIKKGILQTVKVVDELVKVMGRHKP |
| | ENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSI |
| | DNKVLTRSDKNRGKSDNVPSEEVVKKMKNYWRQLLNAKLITQRKFD |
| | NLTKAERGGLSELDKAGFIKRQLVETRQITKHVAQILDSRMNTKYDEN |
| | DKLIREVKVITLKSKLVSDFRKDFQFYKVREINNYHHAHDAYLNAVV |
| | GTALIKKYPKLESEFVYGDYKVYDVRKMIAKSEQEIGKATAKYFFYSN |
| | IMNFFKTEITLANGEIRKRPLIETNGETGEIVWDKGRDFATVRKVLSMP |
| | QVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKG |
| | YKEVKKDLIIKLPKYSLFELENGRKRMLASAGELQKGNELALPSKYVN |
| | FLYLASHYEKLKGSPEDNEQKQLFVEQHKHYLDEIIEQISEFSKR VILA |
| | DANLDKVLSAYNKHRDKPIREQAENIIHLFTLTNLGAPAAFKYFDTTID |
| | RKRYTSTKEVLDATLIHQSITGLYETRIDLSQLGGDSGGSPDRRAAVSH |
| | WQSGGSSGGSSGSKRTADGSEFESPKKKRKVATNFSLLKQAGDVEEN |
| | PGPKRTADGSEFESPKKKRKVQVQLVESGGGLVQPGGSLTLSCTASGF |
| | TLDHYDIGWFRQAPGKEREGVSCINNSDDDTYYADSVKGRFTIFNNAK |
| | DTVYLQMNSLKPEDTAIYYCAEARGCKRGRYEYDFWGQGTQVTVSS |
| | KKKNIEDEYRLHETSKEPDVSLGSTWLSDFPQAWAETGGMGLAVRQA |
| | PLIIPLKATSTPVSIKQYPMSQEARLGIKPHIQRLLDQGILVPCQSPWNTP |
| | LLPVKKPGTNDYRPVQDLREVNKRVEDIHPTVPNPYNLLSGLPPSHQW |
| | YTVLDLKDAFFCLRLHPTSQPLFAFEWRDPEMGISGQLTWTRLPQGFK |
| | NSPTLFNEALHRDLADFRIQHPDLILLQYVDDLLLAATSELDCQQGTR |
| | ALLQTLGNLGYRASAKKAQICQKQVKYLGYLLKEGQRWLTEARKET |
| | VMGQPTPKTPRQLREFLGKAGFCRLFIPGFAEMAAPLYPLTKPGTLFN |
| | WGPDQQKAYQEIKQALLTAPALGLPDLTKPFELFVDEKQGYAKGVLT |
| | QKLGPWRRPVAYLSKKLDPVAAGWPPCLRMVAAIAVLTKDAGKLTM |
| | GQPLVILAPHAVEALVKQPPDRWLSNARMTHYQALLLDTDRVQFGPV |
| | VALNPATLLPLPEEGLQHNCLDILAEAHGTRPDLTDQPLPDADHTWYT |
| | DGSSLLQEGQRKAGAAVTTETEVIWAKALPAGTSAQRAELIALTQALK |
| | MAEGKKLNVYTDSRYAFATAHIHGEIYRRRGWLTSEGKEIKNKDEILA |
| | LLKALFLPKRLSIIHCPGHQKGHSAEARGNRMADQAARKAAITETPDT |
| | STLLIENSSPSGGSKRTADGSEFEPKKKRKV- |
| 8008 | MKRTADGSEFESPKKKRKVDKKYSIGLDIGTNSVGWAVITDEYKVPSK |
| | KFKVLGNTDRHSIKKNLIGALLFDSGETAEATRLKRTARRRYTRRKNR |
| | ICYLQEIFSNEMAKVDDSFFHRLEESFLVEEDKKHERHPIFGNIVDEVA |
| | YHEKYPTIYHLRKKLVDSTDKADLRLIYLALAHMIKFRGHFLIEGDLNP |
| | DNSDVDKLFIQLVQTYNQLFEENPINASGVDAKAILSARLSKSRRLENL |
| | IAQLPGEKKNGLFGNLIALSLGLTPNFKSNFDLAEDAKLQLSKDTYDD |
| | DLDNLLAQIGDQYADLFLAAKNLSDAILLSDILRVNTEITKAPLSASMI |
| | KRYDEHHQDLTLLKALVRQQLPEKYKEIFFDQSKNGYAGYIDGGASQ |
| | EEFYKFIKPILEKMDGTEELLVKLNREDLLRKQRTFDNGSIPHQIHLGEL |
| | HAILRRQEDFYPFLKDNREKIEKILTFRIPYYVGPLARGNSRFAWMTRK |
| | SEETITPWNFEEVVDKGASAQSFIERMTNFDKNLPNEKVLPKHSLLYE |
| | YFTVYNELTKVKYVTEGMRKPAFLSGEQKKAIVDLLFKTNRKVTVKQ |
| | LKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWG |
| | RLSRKLINGIRDKQSGKTILDFLKSDGFANRNFMQLIHDDSLTFKEDIQ |
| | KAQVSGQGDSLHEHIANLAGSPAIKKGILQTVKVVDELVKVMGRHKP |
| | ENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSI |
| | DNKVLTRSDKNRGKSDNVPSEEVVKKMKNYWRQLLNAKLITQRKFD |
| | NLTKAERGGLSELDKAGFIKRQLVETRQITKHVAQILDSRMNTKYDEN |
| | DKLIREVKVITLKSKLVSDFRKDFQFYKVREINNYHHAHDAYLNAVV |
| | GTALIKKYPKLESEFVYGDYKVYDVRKMIAKSEQEIGKATAKYFFYSN |
| | IMNFFKTEITLANGEIRKRPLIETNGETGEIVWDKGRDFATVRKVLSMP |
| | QVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKG |
| | YKEVKKDLIIKLPKYSLFELENGRKRMLASAGELQKGNELALPSKYVN |
| | FLYLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTTID |
| | RKRYTSTKEVLDATLIHQSITGLYETRIDLSQLGGDSGGSSGGSSGSQV |
| | QLVESGGGLVQPGGSLTLSCTASGFTLDHYDIGWFRQAPGKEREGVSC |
| | INNSDDDTYYADSVKGRFTIFNNAKDTVYLQMNSLKPEDTAIYYCAEA |
| | RGCKRGRYEYDFWGQGTQVTVSSKKKKRTADGSEFESPKKKRKVAT |
| | NFSLLKQAGDVEENPGPKRTADGSEFESPKKKRKVGGSPDRKAAVSH |
| | WQSSGGSSGGSSGSNIEDEYRLHETSKEPDVSLGSTWLSDFPQAWAET |
| | GGMGLAVRQAPLIIPLKATSTPVSIKQYPMSQEARLGIKPHIQRLLDQGI |
| | LVPCQSPWNTPLLPVKKPGTNDYRPVQDLREVNKRVEDIHPTVPNPYN |
| | LLSGLPPSHQWYTVLDLKDAFFCLRLHPTSQPLFAFEWRDPEMGISGQ |
| | LTWTRLPQGFKNSPTLFNEALHRDLADFRIQHPDLILLQYVDDLLLAA |
| | TSELDCQQGTRALLQTLGNLGYRASAKKAQICQKQVKYLGYLLKEGQ |
| | RWLTEARKETVMGQPTPKTPRQLREFLGKAGFCRLFIPGFAEMAAPLY |
| | PLTKPGTLFNWGPDQQKAYQEIKQALLTAPALGLPDLTKPFELFVDEK |
| | QGYAKGVLTQKLGPWRRPVAYLSKKLDPVAAGWPPCLRMVAAIAVL |
| | TKDAGKLTMGQPLVILAPHAVEALVKQPPDRWLSNARMTHYQALLL |
| | DTDRVQFGPVVALNPATLLPLPEEGLQHNCLDILAEAHGTRPDLTDQP |
| | LPDADHTWYTDGSSLLQEGQRKAGAAVTTETEVIWAKALPAGTSAQR |
| | AELIALTQALKMAEGKKLNVYTDSRYAFATAHIHGEIYRRRGWLTSE |
| | GKEIKNKDEILALLKALFLPKRLSIIHCPGHQKGHSAEARGNRMADQA |
| | ARKAAITETPDTSTLLIENSSPSGGSKRTADGSEFEPKKKRKV |

TABLE 21
Amino acid sequences of exemplary split prime editor systems
having the DNA binding domain fused to a single-domain antibody
(lacking a self-cleaving peptide)
DNA Binding Domain-Single-domain antibody peptide

| SEQ ID NO: | Sequence |
|---|---|
| 8009 | MKRTADGSEFESPKKKRKVDKKYSIGLDIGTNSVGWAVITDEYKVPSK |
| | KFKVLGNTDRHSIKKNLIGALLFDSGETAEATRLKRTARRRYTRRKNR |
| | ICYLQEIFSNEMAKVDDSFFHRLEESFLVEEDKKHERHPIFGNIVDEVA |
| | YHEKYPTIYHLRKKLVDSTDKADLRLIYLALAHMIKFRGHFLIEGDLNP |
| | DNSDVDKLFIQLVQTYNQLFEENPINASGVDAKAILSARLSKSRRLENL |
| | IAQLPGEKKNGLFGNLIALSLGLTPNFKSNFDLAEDAKLQLSKDTYDD |
| | DLDNLLAQIGDQYADLFLAAKNLSDAILLSDILRVNTEITKAPLSASMI |
| | KRYDEHHQDLTLLKALVRQQLPEKYKEIFFDQSKNGYAGYIDGGASQ |
| | EEFYKFIKPILEKMDGTEELLVKLNREDLLRKQRTFDNGSIPHQIHLGEL |
| | HAILRRQEDFYPFLKDNREKIEKILTFRIPYYVGPLARGNSRFAWMTRK |
| | SEETITPWNFEEVVDKGASAQSFIERMTNFDKNLPNEKVLPKHSLLYE |
| | YFTVYNELTKVKYVTEGMRKPAFLSGEQKKAIVDLLFKTNRKVTVKQ |
| | LKEDYFKKIECFDSVEISGVEDRFNASLGTYHDLLKIIKDKDFLDNEEN |
| | EDILEDIVLTLTLFEDREMIEERLKTYAHLFDDKVMKQLKRRRYTGWG |
| | RLSRKLINGIRDKQSGKTILDFLKSDGFANRNFMQLIHDDSLTFKEDIQ |
| | KAQVSGQGDSLHEHIANLAGSPAIKKGILQTVKVVDELVKVMGRHKP |
| | ENIVIEMARENQTTQKGQKNSRERMKRIEEGIKELGSQILKEHPVENTQ |
| | LQNEKLYLYYLQNGRDMYVDQELDINRLSDYDVDAIVPQSFLKDDSI |
| | DNKVLTRSDKNRGKSDNVPSEEVVKKMKNYWRQLLNAKLITQRKFD |
| | NLTKAERGGLSELDKAGFIKRQLVETRQITKHVAQILDSRMNTKYDEN |
| | DKLIREVKVITLKSKLVSDFRKDFQFYKVREINNYHHAHDAYLNAVV |
| | GTALIKKYPKLESEFVYGDYKVYDVRKMIAKSEQEIGKATAKYFFYSN |
| | IMNFFKTEITLANGEIRKRPLIETNGETGEIVWDKGRDFATVRKVLSMP |
| | QVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKG |
| | YKEVKKDLIIKLPKYSLFELENGRKRMLASAGELQKGNELALPSKYVN |
| | FLYLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTTID |
| | RKRYTSTKEVLDATLIHQSITGLYETRIDLSQLGGDSGGSPDRRAAVSH |
| | WQSGGSSGGSSGSKRTADGSEFESPKKKRKV |
| 8010 | KRTADGSEFESPKKKRKVGGSPDRVRAVSHWSSGGSSGGSSGSNIEDE |
| | YRLHETSKEPDVSLGSTWLSDFPQAWAETGGMGLAVRQAPLIIPLKAT |
| | STPVSIKQYPMSQEARLGIKPHIQRLLDQGILVPCQSPWNTPLLPVKKP |
| | GTNDYRPVQDLREVNKRVEDIHPTVPNPYNLLSGLPPSHQWYTVLDL |
| | KDAFFCLRLHPTSQPLFAFEWRDPEMGISGQLTWTRLPQGFKNSPTLF |
| | NEALHRDLADFRIQHPDLILLQYVDDLLLAATSELDCQQGTRALLQTL |
| | GNLGYRASAKKAQICQKQVKYLGYLLKEGQRWLTEARKETVMGQPT |
| | PKTPRQLREFLGKAGFCRLFIPGFAEMAAPLYPLTKPGTLFNWGPDQQ |
| | KAYQEIKQALLTAPALGLPDLTKPFELFVDEKQGYAKGVLTQKLGPW |
| | RRPVAYLSKKLDPVAAGWPPCLRMVAAIAVLTKDAGKLTMGQPLVIL |
| | APHAVEALVKQPPDRWLSNARMTHYQALLLDTDRVQFGPVVALNPA |
| | TLLPLPEEGLQHNCLDILAEAHGTRPDLTDQPLPDADHTWYTDGSSLL |
| | QEGQRKAGAAVTTETEVIWAKALPAGTSAQRAELIALTQALKMAEGK |
| | KLNVYTDSRYAFATAHIHGEIYRRRGWLTSEGKEIKNKDEILALLKAL |
| | FLPKRLSIIHCPGHQKGHSAEARGNRMADQAARKAAITETPDTSTLLIE |
| | NSSPSGGSKRTADGSEFEPKKKRKV |
| 8011 | KRTADGSEFESPKKKRKVGGSPDRKAAVSHWQSSGGSSGGSSGSNIED |
| | EYRLHETSKEPDVSLGSTWLSDFPQAWAETGGMGLAVRQAPLIIPLKA |
| | TSTPVSIKQYPMSQEARLGIKPHIQRLLDQGILVPCQSPWNTPLLPVKKP |
| | GTNDYRPVQDLREVNKRVEDIHPTVPNPYNLLSGLPPSHQWYTVLDL |
| | KDAFFCLRLHPTSQPLFAFEWRDPEMGISGQLTWTRLPQGFKNSPTLF |
| | NEALHRDLADFRIQHPDLILLQYVDDLLLAATSELDCQQGTRALLQTL |
| | GNLGYRASAKKAQICQKQVKYLGYLLKEGQRWLTEARKETVMGQPT |
| | PKTPRQLREFLGKAGFCRLFIPGFAEMAAPLYPLTKPGTLFNWGPDQQ |
| | KAYQEIKQALLTAPALGLPDLTKPFELFVDEKQGYAKGVLTQKLGPW |
| | RRPVAYLSKKLDPVAAGWPPCLRMVAAIAVLTKDAGKLTMGQPLVIL |
| | APHAVEALVKQPPDRWLSNARMTHYQALLLDTDRVQFGPVVALNPA |
| | TLLPLPEEGLQHNCLDILAEAHGTRPDLTDQPLPDADHTWYTDGSSLL |
| | QEGQRKAGAAVTTETEVIWAKALPAGTSAQRAELIALTQALKMAEGK |
| | KLNVYTDSRYAFATAHIHGEIYRRRGWLTSEGKEIKNKDEILALLKAL |
| | FLPKRLSIIHCPGHQKGHSAEARGNRMADQAARKAAITETPDTSTLLIE |
| | NSSPSGGSKRTADGSEFEPKKKRKV |

TABLE 20
Amino acid sequences of exemplary split prime editor systems
having the DNA polymerase domain fused to a single-domain
antibody (lacking a self-cleaving peptide)
(SEQ ID No. provided in left column)

| | |
|---|---|
| SEQ | |
| ID | |
| NO: | Sequence |

DNA Polymerase Domain-Single-domain antibody peptide

| | |
|---|---|
| 8012 | KRTADGSEFESPKKKRKVGGSQVQLVESGGGLVQPGGSLTLSCTASGFTL |
| | DHYDIGWFRQAPGKEREGVSCINNSDDDTYYADSVKGRFTIFNNAKDTV |
| | YLQMNSLKPEDTAIYYCAEARGCKRGRYEYDFWGQGTQVTVSSKKKNIE |
| | DEYRLHETSKEPDVSLGSTWLSDFPQAWAETGGMGLAVRQAPLIIPLKAT |
| | STPVSIKQYPMSQEARLGIKPHIQRLLDQGILVPCQSPWNTPLLPVKKPGT |
| | NDYRPVQDLREVNKRVEDIHPTVPNPYNLLSGLPPSHQWYTVLDLKDAF |
| | FCLRLHPTSQPLFAFEWRDPEMGISGQLTWTRLPQGFKNSPTLFNEALHR |
| | DLADFRIQHPDLILLQYVDDLLLAATSELDCQQGTRALLQTLGNLGYRAS |
| | AKKAQICQKQVKYLGYLLKEGQRWLTEARKETVMGQPTPKTPRQLREFL |
| | GKAGFCRLFIPGFAEMAAPLYPLTKPGTLFNWGPDQQKAYQEIKQALLTA |
| | PALGLPDLTKPFELFVDEKQGYAKGVLTQKLGPWRRPVAYLSKKLDPVA |
| | AGWPPCLRMVAAIAVLTKDAGKLTMGQPLVILAPHAVEALVKQPPDRW |
| | LSNARMTHYQALLLDTDRVQFGPVVALNPATLLPLPEEGLQHNCLDILAE |
| | AHGTRPDLTDQPLPDADHTWYTDGSSLLQEGQRKAGAAVTTETEVIWA |
| | KALPAGTSAQRAELIALTQALKMAEGKKLNVYTDSRYAFATAHIHGEIY |
| | RRRGWLTSEGKEIKNKDEILALLKALFLPKRLSIIHCPGHQKGHSAEARGN |
| | RMADQAARKAAITETPDTSTLLIENSSPSGGSKRTADGSEFEPKKKRKV |

Compatible DNA binding domain-peptide tag peptides

| | |
|---|---|
| 8013 | MKRTADGSEFESPKKKRKVDKKYSIGLDIGTNSVGWAVITDEYKVPSKK |
| | FKVLGNTDRHSIKKNLIGALLFDSGETAEATRLKRTARRRYTRRKNRICY |
| | LQEIFSNEMAKVDDSFFHRLEESFLVEEDKKHERHPIFGNIVDEVAYHEKY |
| | PTIYHLRKKLVDSTDKADLRLIYLALAHMIKFRGHFLIEGDLNPDNSDVD |
| | KLFIQLVQTYNQLFEENPINASGVDAKAILSARLSKSRRLENLIAQLPGEK |
| | KNGLFGNLIALSLGLTPNFKSNFDLAEDAKLQLSKDTYDDDLDNLLAQIG |
| | DQYADLFLAAKNLSDAILLSDILRVNTEITKAPLSASMIKRYDEHHQDLTL |
| | LKALVRQQLPEKYKEIFFDQSKNGYAGYIDGGASQEEFYKFIKPILEKMD |
| | GTEELLVKLNREDLLRKQRTFDNGSIPHQIHLGELHAILRRQEDFYPFLKD |
| | NREKIEKILTFRIPYYVGPLARGNSRFAWMTRKSEETITPWNFEEVVDKG |
| | ASAQSFIERMTNFDKNLPNEKVLPKHSLLYEYFTVYNELTKVKYVTEGM |
| | RKPAFLSGEQKKAIVDLLFKTNRKVTVKQLKEDYFKKIECFDSVEISGVE |
| | DRFNASLGTYHDLLKIIKDKDFLDNEENEDILEDIVLTLTLFEDREMIEERL |
| | KTYAHLFDDKVMKQLKRRRYTGWGRLSRKLINGIRDKQSGKTILDFLKS |
| | DGFANRNFMQLIHDDSLTFKEDIQKAQVSGQGDSLHEHIANLAGSPAIKK |
| | GILQTVKVVDELVKVMGRHKPENIVIEMARENQTTQKGQKNSRERMKRI |
| | EEGIKELGSQILKEHPVENTQLQNEKLYLYYLQNGRDMYVDQELDINRLS |
| | DYDVDAIVPQSFLKDDSIDNKVLTRSDKNRGKSDNVPSEEVVKKMKNY |
| | WRQLLNAKLITQRKFDNLTKAERGGLSELDKAGFIKRQLVETRQITKHVA |
| | QILDSRMNTKYDENDKLIREVKVITLKSKLVSDFRKDFQFYKVREINNYH |
| | HAHDAYLNAVVGTALIKKYPKLESEFVYGDYKVYDVRKMIAKSEQEIGK |
| | ATAKYFFYSNIMNFFKTEITLANGEIRKRPLIETNGETGEIVWDKGRDFAT |
| | VRKVLSMPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFL |
| | EAKGYKEVKKDLIIKLPKYSLFELENGRKRMLASAGELQKGNELALPSKY |
| | VNFLYLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTTIDRK |
| | RYTSTKEVLDATLIHQSITGLYETRIDLSQLGGDSGGSSGGSSGSPDRVRA |
| | VSHWSSGGSKRTADGSEFESPKKKRKV |
| 8009 | MKRTADGSEFESPKKKRKVDKKYSIGLDIGTNSVGWAVITDEYKVPSKK |
| | FKVLGNTDRHSIKKNLIGALLFDSGETAEATRLKRTARRRYTRRKNRICY |
| | LQEIFSNEMAKVDDSFFHRLEESFLVEEDKKHERHPIFGNIVDEVAYHEKY |
| | PTIYHLRKKLVDSTDKADLRLIYLALAHMIKFRGHFLIEGDLNPDNSDVD |
| | KLFIQLVQTYNQLFEENPINASGVDAKAILSARLSKSRRLENLIAQLPGEK |
| | KNGLFGNLIALSLGLTPNFKSNFDLAEDAKLQLSKDTYDDDLDNLLAQIG |
| | DQYADLFLAAKNLSDAILLSDILRVNTEITKAPLSASMIKRYDEHHQDLTL |
| | LKALVRQQLPEKYKEIFFDQSKNGYAGYIDGGASQEEFYKFIKPILEKMD |
| | GTEELLVKLNREDLLRKQRTFDNGSIPHQIHLGELHAILRRQEDFYPFLKD |
| | NREKIEKILTFRIPYYVGPLARGNSRFAWMTRKSEETITPWNFEEVVDKG |
| | ASAQSFIERMTNFDKNLPNEKVLPKHSLLYEYFTVYNELTKVKYVTEGM |
| | RKPAFLSGEQKKAIVDLLFKTNRKVTVKQLKEDYFKKIECFDSVEISGVE |
| | DRFNASLGTYHDLLKIIKDKDFLDNEENEDILEDIVLTLTLFEDREMIEERL |
| | KTYAHLFDDKVMKQLKRRRYTGWGRLSRKLINGIRDKQSGKTILDFLKS |
| | DGFANRNFMQLIHDDSLTFKEDIQKAQVSGQGDSLHEHIANLAGSPAIKK |
| | GILQTVKVVDELVKVMGRHKPENIVIEMARENQTTQKGQKNSRERMKRI |
| | EEGIKELGSQILKEHPVENTQLQNEKLYLYYLQNGRDMYVDQELDINRLS |
| | DYDVDAIVPQSFLKDDSIDNKVLTRSDKNRGKSDNVPSEEVVKKMKNY |
| | WRQLLNAKLITQRKFDNLTKAERGGLSELDKAGFIKRQLVETRQITKHVA |
| | QILDSRMNTKYDENDKLIREVKVITLKSKLVSDFRKDFQFYKVREINNYH |
| | HAHDAYLNAVVGTALIKKYPKLESEFVYGDYKVYDVRKMIAKSEQEIGK |
| | ATAKYFFYSNIMNFFKTEITLANGEIRKRPLIETNGETGEIVWDKGRDFAT |
| | VRKVLSMPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFL |
| | EAKGYKEVKKDLIIKLPKYSLFELENGRKRMLASAGELQKGNELALPSKY |
| | VNFLYLASHYEKLKGSPEDNEQKQLFVEQHKHYLDEIIEQISEFSKR VILA |
| | DANLDKVLSAYNKHRDKPIREQAENIIHLFTLTNLGAPAAFKYFDTTIDRK |
| | RYTSTKEVLDATLIHQSITGLYETRIDLSQLGGDSGGSPDRRAAVSHWQS |
| | GGSSGGSSGSKRTADGSEFESPKKKRKV |

Disclosed herein, in some embodiments, are compositions, systems, and methods using a split prime editor. In some embodiments, the split prime editor comprises a DNA binding domain and a DNA polymerase domain, wherein the split prime editor comprises a first polypeptide comprising a first amino acid sequence and a second polypeptide comprising a second amino acid sequence. In some embodiments, the first amino acid sequence forms at least a portion of the DNA binding domain. In certain embodiments, the first amino acid sequence forms the DNA binding domain.

In some embodiments, the first amino acid sequence forms at least a portion of the DNA polymerase domain. In certain embodiments, the first amino acid sequence forms the DNA polymerase domain.

In some embodiments, the first amino acid sequence forms at least a portion of the DNA binding domain. In certain embodiments, the first amino acid sequence forms the DNA binding domain.

In some embodiments, the first amino acid sequence forms the DNA binding domain and a portion of the DNA polymerase domain.

In some embodiments, the first amino acid sequence forms the DNA polymerase domain and a portion of the DNA binding domain.

In some embodiments, the second amino acid sequence forms at least a portion of the DNA binding domain. In certain embodiments, the second amino acid sequence forms the DNA binding domain.

In some embodiments, the second amino acid sequence forms at least a portion of the DNA polymerase domain. In certain embodiments, the second amino acid sequence forms the DNA polymerase domain.

In some embodiments, the second amino acid sequence forms the DNA binding domain and a portion of the DNA polymerase domain.

In some embodiments, the second amino acid sequence forms the DNA polymerase domain and a portion of the DNA binding domain.

In some embodiments, the first polypeptide and the second polypeptide are joined by a self-cleaving peptide. In some embodiments, the first polypeptide and the second polypeptide are covalently linked by a self-cleaving peptide. In some embodiments, the C-terminus of the second polypeptide and the N-terminus of the first polypeptide are linked by a self-cleaving peptide. In some embodiments, the N-terminus of the second polypeptide and the C-terminus of the first polypeptide are linked by a self-cleaving peptide. In some embodiments, the self-cleaving peptide has a sequence as set forth in Table 19 (e.g., 2A peptide, such as a P2A, E2A, T2A, a F2A peptide, a BmCPV2A peptide, or a BmFV2A peptide)

TABLE 19
Exemplary self-cleaving peptide sequence

| | Self- | |
|---|---|---|
| SEQ | cleaving | |
| ID NO: | peptide | Sequence |
| 8004 | P2A | ATNFSLLKQAGDVEENPGP |
| 8014 | E2A | QCTNYALLKLAGDVESNPGP |
| 8015 | T2A | EGRGSLLTCGDVEENPGP |
| 8016 | F2A | VKQTLNFDLLKLAGDVESNPGP |
| 8017 | BmCPV2A | RTAFDFQQDVFRSNYDLLKLSGDIESNPGP |
| 8018 | BmFV2A | PSIGNVARTLTRAKIEDELIRAGIESNPGP |

In certain embodiments, the first polypeptide and the second polypeptide are configured to passively assemble in a host cell to form the split prime editor.

In some embodiments, the first polypeptide has affinity for the second polypeptide.

In some embodiments, the second polypeptide has affinity for the first polypeptide.

In some embodiments, the first polypeptide comprises a single-domain antibody, the second polypeptide comprises a peptide tag, and the single-domain antibody is configured to bind to the peptide tag. In some embodiments, the first polypeptide comprises a peptide tag, the second polypeptide comprises a single-domain antibody, and the single-domain antibody is configured to bind to the peptide tag.

In some embodiments, the first polypeptide comprises a single-domain antibody (e.g., a NANOBODY®). In some embodiments, the single-domain antibody has the amino acid sequence disclosed in Table 17).

In some embodiments, the second polypeptide comprises a single-domain antibody (e.g., a NANOBODY®). In some embodiments, the single-domain antibody has the amino acid sequence in Table 17).

In some embodiments, the first polypeptide comprises a peptide tag (e.g., a SpotTag®, a BC2 tag) configured to bind to a single-domain antibody. In some embodiments, the second polypeptide comprises a peptide tag (e.g., a SpotTag®, a BC2 tag) configured to bind to a single-domain antibody. In some embodiments, the peptide tag has any one of the amino acid sequences of in Table 16). In some embodiments, the peptide tag is a SpotTag®, a BC2 tag, or a variant thereof.

In some embodiments, the first polypeptide and second polypeptide undergo directed evolution to, for example, increase affinity of the first polypeptide and the second polypeptide to each other. As used herein, “directed evolution” encompasses methods to design proteins with desirable functions and characteristics. In some embodiments, directed evolution generates random mutations in the gene of interest and requires no protein structure information. Directed evolution mimics natural evolution by imposing stringent selection and screening methodologies to identify proteins with optimized functionality, including affinity, binding, catalytic properties, thermal and environmental stability. Exemplary methods for performing directed evolution are described below in Table A. In some embodiments, the first and/or second polypeptide have undergone one of the methods of directed evolution listed in Table A.

The polypeptides that have undergone directed evolution may have an editing efficiency of at least about 5%, 10%, 15%, 20%, 25%, 30%, 35%, 40%, 45%, 50%, 55%, 60%, 65%, 70%, 75%, 80%, 85%, 90%, or 95% for example, when transfected into cells. The polypeptides that have undergone directed evolution may have an editing efficiency of at least about 5%, 10%, 15%, 20%, 25%, 30%, 35%, 40%, 45%, 50%, 55%, 60%, 65%, 70%, 75%, 80%, 85%, 90%, or 95% for example, when transduced into cells.

TABLE A
Exemplary Methods of Directed Evolution

| | |
|---|---|
| Method | Method Information |

Random

| | |
|---|---|
| Error-prone PCR | Employs polymerase to generate mutations |
| | by imposing nucleotide incorporation error |
| | during DNA replication |
| Sequence Saturation | Generates multiple, random single nucleotide |
| Mutagenesis (SeSaM) | mutations in a given gene sequence |
| Site-directed mutagenesis | Enables replication by use of primers with |
| | modified bases resulting in mismatch and |
| | variation at a given position |
| Cassette mutagenesis | Gene cassette or oligonucleotide used for |
| | site-directed mutagenesis |

Recombination

| | |
|---|---|
| DNA shuffling | Mutation and recombination of homologous |
| | genes |
| Staggered Extension | Modified annealing and extension steps |
| Protocol (StEP) | generating staggered fragments |
| Incremental Truncation for | Random recombination between two gene |
| the Creation of Hybrid | fragments |
| Enzymes (ITCHY) | |
| Random Chimeragenesis | Gene family shuffling with multiple |
| on Transient Templates | crossover events for every gene |
| (RACHITT) | |

In certain embodiments, the split prime editor further comprises an affinity moiety that has affinity for either the DNA binding domain or the DNA polymerase domain. In some embodiments, the affinity moiety has affinity for the DNA binding domain. In some embodiments, the affinity moiety has affinity for the DNA polymerase domain.

In some embodiments, the split prime editor comprises a peptide tag/antibody or antibody fragment system that facilities localization of the first and second polypeptides.

In some embodiments, the first polypeptide further comprises a peptide tag. In some embodiments, the second polypeptide further comprises a single domain antibody sequence. In some embodiments, the first polypeptide further comprises a single domain antibody sequence. In some embodiments, the second polypeptide further comprises a peptide tag.

Exemplary peptide tag/antibody or antibody fragment systems include the Spot-Tag® and BC2 systems. These systems include short peptide tag that binds to an antibody or antibody fragment. In some embodiments, the peptide tag is less than 50 amino acids (e.g., less than 49 amino acids, less than 48 amino acids, less than 47 amino acids, less than 46 amino acids, less than 45 amino acids, less than 44 amino acids, less than 43 amino acids, less than 42 amino acids, less than 41 amino acids, less than 40 amino acids, less than 39 amino acids, less than 38 amino acids, less than 37 amino acids, less than 36 amino acids, less than 35 amino acids, less than 34 amino acids, less than 33 amino acids, less than 32 amino acids, less than 31 amino acids, less than 30 amino acids, less than 29 amino acids, less than 28 amino acids, less than 27 amino acids, less than 26 amino acids, less than 25 amino acids, less than 24 amino acids, less than 23 amino acids, less than 22 amino acids, less than 21 amino acids, less than 20 amino acids, less than 19 amino acids, less than 18 amino acids, less than 17 amino acids, less than 16 amino acids, less than 15 amino acids, less than 14 amino acids, less than 13 amino acids, less than 12 amino acids, less than 11 amino acids, less than 10 amino acids, less than 9 amino acids, less than 8 amino acids, less than 7 amino acids, less than 6 amino acids, less than 5 amino acids, less than 4 amino acids, or less than 3 amino acids) in length.

The peptide tag may comprise any sequence set forth in Table 16. The single domain antibody sequence may comprise the sequence set forth in Table 17.

In some embodiments, the DNA binding domain and/or the DNA polymerase domain comprises a peptide tag (e.g., a SpotTag®, a BC2 tag, or variants thereof) that is configured to bind to the affinity moiety (e.g., an affinity moiety).

In some embodiments, the affinity moiety comprises an antibody or fragment thereof (e.g., a NANOBODY®). In some embodiments, the affinity moiety comprises a single-domain antibody (e.g., a NANOBODY®).

TABLE 17
Exemplary single-domain antibody sequence

| SEQ | |
|---|---|
| ID NO: | Single-domain antibody sequence |
| 8002 | QVQLVESGGGLVQPGGSLTLSCTASGFTLDHYDIGWFRQAP |
| | GKEREGVSCINNSDDDTYYADSVKGRFTIFNNAKDTVYLQM |
| | NSLKPEDTAIYYCAEARGCKRGRYEYDFWGQGTQVTVSSKK |
| | K |

TABLE 16
Exemplary peptide tag sequences

| SEQ ID NO: | Peptide Tag sequence |
|---|---|
| 8003 | PDRVRAVSHWS |
| 8019 | PDRKAAVSHWQ |
| 8020 | PDRRAAVSHWQ |

In certain embodiments, the affinity moiety has affinity for the DNA binding domain.

In certain embodiments, the affinity moiety has affinity for the DNA polymerase domain.

In some embodiments, wherein the affinity moiety is fused to the first polypeptide and has affinity for the second amino acid sequence.

In some embodiments, the affinity moiety is fused to the second polypeptide and has affinity for the first amino acid sequence.

The polypeptides including an affinity moiety may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transfected into cells. The polypeptides including an affinity moiety may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transduced into cells.

In some embodiments, the first polypeptide comprises a SpyTag peptide sequence and the second polypeptide comprises a SpyCatcher peptide sequence. The SpyCatcher-SpyTag system is a method for protein ligation. The system is based on a modified domain from a Streptococcus pyogenes surface protein (SpyCatcher), which recognizes a cognate 13-amino-acid peptide (SpyTag). Upon recognition, the SpyCatcher and SpyTag form a covalent isopeptide bond between the side chains of a lysine in SpyCatcher and an aspartate in SpyTag. This technology may be used, among other applications, to create covalently stabilized multi-protein complexes, to label proteins (e.g., for microscopy). The SpyTag system is versatile as the tag is a short, unfolded peptide that can be genetically fused to exposed positions in target proteins. Similarly, SpyCatcher can be fused to reporter proteins such as GFP, and to epitope or purification tags. Exemplary SpyCatcher Reagents are shown in Table 4.

TABLE 4
Exemplary SpyCatcher Reagents

| | | Bio-Rad |
|---|---|---|
| | | Catalog Number |
| Monovalent Format | | |
| SpyCatcher2 | SpyCatcher2 protein | TZC001 |
| SpyCatcher2-CYS | SpyCatcher2 with an engineered | TZC001CYS |
| | cysteine residue; use for site- | |
| | specific chemical conjugation | |
| | to a label of choice | |
| SpyCatcher2: Biotin | SpyCatcher2 conjugated to biotin | |
| SpyCatcher2: HRP | SpyCatcher2 conjugated to HRP | |
| SpyCatcher2: PE | Spycatcher2 conjugated to RPE | |
| SpyCatcher3 | SpyCatcher3 protein | TZC025 |
| SpyCatcher3-CYS | SpyCatcher3 with an engineered | TZC025CYS |
| | cysteine residue; use for site- | |
| | specific conjugation to a label | |
| | of choice | |
| Bivalent Format | | |
| BiSpyCatcher2 | BiSpyCatcher2 protein | TZC002 |
| BiSpyCatcher2-CYS | BiSpyCatcher2 with one engineered | TZC002CYS |
| | cysteine residue; use for site- | |
| | specific conjugation to a label | |
| | of choice | |
| BiSpyCatcher2-CYS3 | BiSpyCatcher2 with three engineered | TZC002CYS3 |
| | cysteine residues; use for site- | |
| | specific conjugation to a label | |
| | of choice | |
| BiSpyCatcher2: Biotin | BiSpyCatcher2 conjugated to biotin | |
| BiSpyCatcher2: HRP | BiSpyCatcher2 conjugated to HRP | |
| BiSpyCatcher2: PE | BiSpyCatcher2 conjugated to RPE | |
| Ig-like Format | | |
| hIgG1-FcSpyCatcher3 | SpyCatcher3 fused to the hinge | TZC009 |
| | region, CH2, and CH3 of human | |
| | IgG1 | |
| hIgG1- | hIgG1-FcSpyCatcher3 conjugated | |
| FcSpyCatcher3: Biotin | to biotin | |
| hIgG1-FcSpyCatcher3: | hIgG1-FcSpyCatcher3 conjugated | |
| HRP | to HRP | |
| hIgG2-FcSpyCatcher3 | SpyCatcher3 fused to the hinge | TZC016 |
| | region, CH2, and CH3 of human | |
| | IgG2 | |
| hIgG3-FcSpyCatcher3 | SpyCatcher3 fused to the hinge | TZC017 |
| | region, CH2, and CH3 of human | |
| | IgG3 | |
| hIgG4-FcSpyCatcher3 | SpyCatcher3 fused to the hinge | TZC018 |
| | region, CH2, and CH3 of human | |
| | IgG4 | |
| hIgG4-Pro- | SpyCatcher3 fused to the hinge | TZC019 |
| FcSpyCatcher3 | region, CH2, and CH3 of human | |
| | IgG4-Pro (S228P) | |
| hIgA-FcSpyCatcher3 | SpyCatcher3 fused to the hinge | TZC020 |
| | region, CH2, and CH3 of human | |
| | IgA | |
| mIgG2a-FcSpyCatcher3 | SpyCatcher3 fused to the hinge | TZC012 |
| | region, CH2, and CH3 of mouse | |
| | IgG2a | |
| rbIgG-FcSpyCatcher3 | SpyCatcher3 fused to the hinge | TZC013 |
| | region, CH2, and CH3 of rabbit | |
| | IgG | |

Orthogonal systems to the SpyCatcher-SpyTag system include SnoopTag-SnoopCatcher system, SdyTag-SdyCatcher system, DogTag-DogCatcher system, SpyTag-SpyDock system, and isopeptag-Pilin-C system.

The polypeptides including the SpyCatcher-SpyTag system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transfected into cells. The polypeptides including the SpyCatcher-SpyTag system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transduced into cells.

In certain embodiments, first polypeptide comprises a SnoopTag peptide sequence and the second polypeptide comprises a SnoopCatcher peptide sequence. The SnoopTag-SnoopCatcher system is derived from the adhesin RrgA of Streptococcus pneumonia. The peptide SnoopTag forms a spontaneous isopeptide bond to its protein partner SnoopCatcher.

The polypeptides including the SnoopTag-SnoopCatcher system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transfected into cells. The polypeptides including the SnoopTag-SnoopCatcher system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transduced into cells.

In some embodiments, the first polypeptide comprises a SdyTag peptide sequence and the second polypeptide comprises a SdyCatcher peptide sequence. The Sdy Tag-SdyCatcher system is derived from the Cna protein B-type (CnaB) domain of Streptococcus dysgalactiae.

In certain embodiments, the first polypeptide comprises a DogTag peptide sequence and the second polypeptide comprises a DogCatcher peptide sequence. The DogTag-DogCatcher system is derived from the adhesin RrgA of Streptococcus pneumonia.

The polypeptides including the SdyTag-SdyCatcher system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transfected into cells. The polypeptides including the SdyTag-SdyCatcher system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transduced into cells.

In some embodiments, the first polypeptide comprises a SpyTag peptide sequence and the second polypeptide comprises a SpyDock peptide sequence.

The polypeptides including the SdyTag-SdyDock system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transfected into cells. The polypeptides including the SdyTag-SdyDock system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transduced into cells.

In certain embodiments, the first polypeptide comprises an isopeptag peptide sequence and the second polypeptide comprises a Pilin-C peptide sequence. The isopeptag-Pilin-C system is derived from the pilin protein (Spy0128) of Streptococcus pyogenes.

The polypeptides including the isopeptag-Pilin-C system may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transfected into cells. The polypeptides including the isopeptag-Pilin-C may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transduced into cells.

In some embodiments, the split prime editor comprises a third polypeptide encoding a third amino acid sequence. In certain embodiments, the third amino acid sequence forms at least a portion of the DNA binding domain and/or the DNA polymerase domain.

In various embodiments, the split prime editors described herein may be delivered to cells as two or more fragments which become assembled inside the cell (either by passive assembly, or by active assembly, such as using split intein sequences) into a reconstituted split prime editor. In some cases, the self-assembly may be passive whereby the two or more split prime editor fragments or polypeptides associate inside the cell covalently or non-covalently to reconstitute the split prime editor. In other cases, the self-assembly may be catalyzed by dimerization domains installed on each of the fragments. In still other cases, the self-assembly may be catalyzed by split intein sequences installed on each of the split prime editor fragments.

Once delivered or expressed within a cell, the split intein domains of the different fragments associate and bind to one another, and then undergo trans-splicing, which results in the excision of the split-intein domains from each of the fragments, and a concomitant formation of a peptide bond between the fragments, thereby restoring the split prime editor.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any split prime editor polypeptide component. In some embodiments, the intein-N and intein-C of a split intein can associate non-covalently to form an active intein and catalyze a-trans splicing reaction. In some embodiments, the trans splicing reaction excises the two intein sequences and links the two extein sequences with a peptide bond. As a result, the intein-N and the intein-C are spliced out, and a protein domain linked to the intein-N is fused to a protein domain linked to the intein-C essentially in same way as a contiguous intein does. In some embodiments, a split-intein is derived from a eukaryotic intein, a bacterial intein, or an archaeal intein. Preferably, the split intein so-derived will possess only the amino acid sequences essential for catalyzing trans-splicing reactions. In some embodiments, an intein-N or an intein-C further comprise one or more amino acid substitutions as compared to a wild type intein-N or wild type intein-C, for example, amino acid substitutions that enhances the trans-splicing activity of the split intein. In some embodiments, the intein-C comprises 4 to 7 contiguous amino acid residues, wherein at least 4 amino acids of which are from the last β-strand of the intein from which it was derived. In some embodiments, the split intein is derived from a Ssp DnaE intein, e.g., Synechocytis sp. PCC6803, or any intein or split intein known in the art, or any functional variants or fragments thereof.

In one embodiment, the split prime editor can be delivered using a split-intein approach. In certain embodiments, the split site is located one or more polypeptide bond sites (i.e., a “split site or split-intein split site”), fused to a split intein, and then delivered to cells as separately-encoded fusion proteins. Once the split-intein fusion proteins (i.e., protein halves) are expressed within a cell, the proteins undergo trans-splicing to form a complete or whole split prime editor with the concomitant removal of the joined split-intein sequences. To take advantage of a split prime editor delivery strategy using split-inteins, the split prime editor needs to be divided at one or more split sites to create at least two separate halves of a split prime editor, each of which may be rejoined inside a cell if each half is fused to a split-intein sequence.

An exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C.

Additional naturally occurring or engineered split-intein sequences are known in the or can be made from whole-intein sequences described herein or those available in the art.

Examples of split-intein sequences can be found in Stevens et al, “A promiscuous split intein with expanded protein engineering applications,” PNAS, 2017, Vol. 114:8538-8543; Iwai et al., “Highly efficient protein trans-splicing by a naturally split DnaE intein from Nostc punctiforme, FEBS Lett, 580:1853-1858, each of which are incorporated herein by reference. Additional split intein sequences can be found, for example, in WO 2013/045632, WO 2014/055782, WO 2016/069774, and EP2877490, the contents each of which are incorporated herein by reference.

In certain embodiments, the first polypeptide comprises a C-terminal intein sequence. In certain embodiments, wherein the second polypeptide comprises a N-terminal intein sequence. In some embodiments, assembly of the first polypeptide and the second polypeptide in a host cell results in fusion of the C-terminal intein sequence and the N-terminal intein sequence to generate a full intein sequence, which then results in splicing and excision of the full intein sequence.

The polypeptides including the intein sequence may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transfected into cells. The polypeptides including the intein sequence have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transduced into cells.

In certain embodiments, the first polypeptide comprises a first affinity moiety and the second polypeptide comprises a second affinity moiety. In some embodiments, the first affinity moiety described herein has affinity for the second affinity moiety described herein.

In some embodiments, the first affinity moiety comprises a C-terminal leucine zipper monomer. In some embodiments, the second affinity moiety comprises an N-terminal leucine zipper monomer. In some embodiments, the C-terminal leucine zipper monomer and the N-terminal leucine zipper monomer forms a dimer in a host cell.

The polypeptides including leucine zippers may have an editing efficiency of at least about 5%, 10%, 15%, 20%, 25%, 30%, 35%, 40%, 45%, 50%, 55%, 60%, 65%, 70%, 75%, 80%, 85%, 90%, or 95% for example, when transfected into cells. The polypeptides including leucine zippers may have an editing efficiency of at least about 5%, 10%, 15%, 20%, 25%, 30%, 35%, 40%, 45%, 50%, 55%, 60%, 65%, 70%, 75%, 80%, 85%, 90%, or 95% for example, when transduced into cells. A benefit of using leucine zipper is to separate the polymerase and nuclease (or a portion of them), and allow them to fit within AAV vectors.

In some embodiments, the first affinity moiety comprises a C-terminal dimerization domain. In some embodiments, the second affinity moiety comprises a N-terminal dimerization domain. In certain embodiments, the C-terminal dimerization domain and the N-terminal dimerization domain form a dimer in a host cell. As used herein, a “dimerization domain” includes any protein domain that facilitates self-association of proteins to form dimers.

The polypeptides including dimerization domains may have an editing efficiency of at least about 5%, 10%, 15%, 20%, 25%, 30%, 35%, or 40%, 45%, 50%, 55%, 60%, 65%, 70%, 75%, 80%, 85%, 90%, or 95% for example, when transfected into cells. The polypeptides including dimerization domains may have an editing efficiency of at least about 5%, 10%, 15%, 20%, 25%, 30%, 35%, 40%, 45%, 50%, 55%, 60%, 65%, 70%, 75%, 80%, 85%, 90%, or 95%, for example, when transduced into cells.

In certain aspects, the prime editor systems described herein comprise a split prime editor comprising a DNA binding domain and a DNA polymerase domain, wherein the split prime editor comprises a first polypeptide comprising a first amino acid sequence, a second polypeptide comprising a second amino acid sequence, and a third polypeptide comprising a third amino acid sequence. The third amino acid sequence may comprise at least a portion of the DNA binding domain and/or at least a portion of the DNA polymerase domain.

### Prime Editing Compositions/Systems

Disclosed herein, in some embodiments, are compositions, systems, and methods using a prime editing composition or system. The term “prime editing composition” or “prime editing system” refers to compositions involved in the method of prime editing as described herein. A prime editing composition may include a split prime editor, e.g., a split prime editor comprising a DNA binding domain and a DNA polymerase domain, wherein the split prime editor comprises a first polypeptide comprising a first amino acid sequence and a second polypeptide comprising a second amino acid sequence. The composition may further include a PEgRNA. A prime editing composition may further comprise additional elements, such as second strand nicking ngRNAs. Components of a prime editing composition may be combined to form a complex for prime editing, or may be kept separately, e.g., for administration purposes.

In some embodiments, a prime editing composition comprises a split prime editor disclosed herein comprising at least two separate polypeptides, wherein at least one of the polypeptides is complexed with a PEgRNA and optionally complexed with a ngRNA. In some embodiments, the prime editing composition comprises a split prime editor comprising a DNA binding domain and a DNA polymerase domain associated with each other through a PERNA. For example, the prime editing composition may comprise a split prime editor comprising a DNA binding domain and a DNA polymerase domain linked to each other by an RNA-protein recruitment aptamer RNA sequence, which is linked to a PERNA. In some embodiments, a prime editing composition comprises a PEgRNA and a polynucleotide, a polynucleotide construct, or a vector that encodes a split prime editor disclosed herein.

In some embodiments, a prime editing composition comprises a PERNA, a ngRNA, and a polynucleotide, a polynucleotide construct, or a vector that encodes a split prime editor disclosed herein. In some embodiments, a prime editing composition comprises multiple polynucleotides, polynucleotide constructs, or vectors, each of which encodes one or more prime editing composition components (e.g., a first amino acid sequence that forms at least a portion of the DNA binding domain and a second amino acid sequence that form at least a portion of the DNA polymerase domain). In some embodiments, the PEgRNA of a prime editing composition is associated with the DNA binding domain, e.g., a Cas9 nickase, of the split prime editor. In some embodiments, the PEgRNA of a prime editing composition complexes with the DNA binding domain of a split prime editor and directs the split prime editor to the target DNA.

In some embodiments, a prime editing composition comprises one or more polynucleotides that encode split prime editor components and/or PERNA or ngRNAs. In some embodiments, a prime editing composition comprises a polynucleotide encoding a split prime editor comprising a DNA binding domain and a DNA polymerase domain. In some embodiments, a prime editing composition comprises (i) a polynucleotide encoding a protein comprising a DNA binding domain and a DNA polymerase domain, and (ii) a PEgRNA or a polynucleotide encoding the PEgRNA. In some embodiments, a prime editing composition comprises (i) a polynucleotide encoding a protein comprising a DNA binding domain and a DNA polymerase domain, (ii) a PERNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a split prime editor, e.g., a Cas9 nickase, (ii) a polynucleotide encoding a DNA polymerase domain of a split prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a split prime editor, e.g., a Cas9 nickase, (ii) a polynucleotide encoding a DNA polymerase domain of a split prime editor, e.g., a reverse transcriptase, (iii) a PEgRNA or a polynucleotide encoding the PEgRNA, and (iv) an ngRNA or a polynucleotide encoding the ngRNA.

In some embodiments, the at least one polynucleotide encoding the DNA binding domain or the polynucleotide encoding the DNA polymerase domain further encodes an additional polypeptide domain, e.g., an RNA-protein recruitment domain, and/or an adapter protein, such as an MS2 coat protein domain, a PP7 adapter protein, a Qβ adapter protein, a F2 adapter protein, a GA adapter protein, a fr adapter protein, a JP501 adapter protein, a M12 adapter protein, a R17 adapter protein, a BZ13 adapter protein, a JP34 adapter protein, a JP500 adapter protein, a KU1 adapter protein, a M11 adapter protein, a MX1 adapter protein, a TW18 adapter protein, a VK adapter protein, a SP adapter protein, a FI adapter protein, a ID2 adapter protein, a NL95 adapter protein, a TW 19 adapter protein, a AP205 adapter protein, a ϕCb5 adapter protein, a ϕCb8r adapter protein, a ϕ12r adapter protein, a ϕCb23r adapter protein, a 7s adapter protein, a PRR1 adapter protein, a leucine zipper monomer, a dimerization domain, an affinity moiety (e.g., antibody (e.g., NANOBODY®)), scaffold protein, a SpyTag peptide sequence, a SpyCatcher peptide sequence, a SnoopTag peptide sequence, a SnoopCatcher peptide sequence, a SdyTag peptide sequence, a SdyCatcher peptide sequence, a DogTag peptide sequence, a DogCatcher peptide sequence, and a SpyDock peptide sequence

In some embodiments, a prime editing composition comprises (i) a polynucleotide encoding a first polypeptide comprising a first amino acid sequence (e.g., the N-terminal half of a split prime editor) and an intein-N and (ii) a polynucleotide encoding a second polypeptide comprising a second amino acid sequence (e.g., the C-terminal half of the split prime editor) and an intein-C. In some embodiments, a prime editing composition comprises (i) a polynucleotide encoding a N-terminal half of the split prime editor and an intein-N(ii) a polynucleotide encoding a C-terminal half of the split prime editor and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, a prime editing composition comprises (i) a polynucleotide encoding a N-terminal portion of a DNA polymerase domain and an intein-N, (ii) a polynucleotide encoding a C-terminal portion of the DNA polymerase domain, an intein-C, and a DNA binding domain.

In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing composition comprises (i) a polynucleotide encoding a N-terminal portion of a DNA polymerase domain and an intein-N, (ii) a polynucleotide encoding a C-terminal portion of the DNA polymerase domain, an intein-C, and a DNA binding domain, and (iii) a PERNA or a polynucleotide encoding the PERNA, and/or (iv) a ngRNA or a polynucleotide encoding the ngRNA.

In some embodiments, a prime editing system comprises one or more polynucleotides encoding one or more split prime editor polypeptides, wherein activity of the prime editing system may be temporally regulated by controlling the timing in which the vectors are delivered. For example, in some embodiments, a polynucleotide encoding the split prime editor and a polynucleotide encoding a PERNA may be delivered simultaneously. For example, in some embodiments, a polynucleotide encoding the split prime editor and a polynucleotide encoding a PERNA may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

### Split Prime Editor Nucleotide Polymerase Domain

In some embodiments, a split prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the split prime editor comprises a DNA-dependent DNA polymerase. For example, a split prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA).

The DNA polymerases can be wild type polymerases from eukaryotic, prokaryotic, archael, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

For synthesis of longer nucleic acid molecules (e.g., nucleic acid molecules longer than about 3-5 Kb in length), at least two DNA polymerases can be employed. In certain embodiments, one of the polymerases can be substantially lacking a 3′ exonuclease activity and the other may have a 3′ exonuclease activity. Such pairings may include polymerases that are the same or different. Examples of DNA polymerases substantially lacking in 3′ exonuclease activity include, but are not limited to, Taq, Tne(exo-), Tma(exo-), Pfu(exo-), Pwo(exo-), exo-KOD and Tth DNA polymerases, and any functional mutants, functional variants and functional fragments thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA polymerase is a Pol IV family DNA polymerase. In some embodiments, the DNA polymerase is an E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA polymerase is a Pol III family DNA polymerase. In some embodiments, the DNA polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UITma).

In some embodiments, a split prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a split prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a split prime editor comprising the engineered RT has improved prime editing efficiency over a split prime editor having a reference naturally occurring RT.

In some embodiments, a split prime editor comprises a virus RT, for example, a retrovirus RT. Non-limiting examples of virus RT include Moloney murine leukemia virus (M-MLV or MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the split prime editor comprises a wild type M-MLV RT. An exemplary sequence of a wild type M-MLV RT is provided in SEQ ID NO: 4448.

In some embodiments, the split prime editor comprises a M-MMLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the wild type M-MMLV RT as set forth in SEQ ID NO: 4448, where X is any amino acid other than the wild type amino acid. In some embodiments, the split prime editor comprises a M-MMLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the wild type M-MMLV RT as set forth in SEQ ID NO: 4448. In some embodiments, the split prime editor comprises a M-MLV RT comprising one or more amino acid substitutions D200N, T330P, L603W, T306K, and W313F as compared to the wild type M-MMLV RT as set forth in SEQ ID NO: 4448. In some embodiments, the split prime editor comprises a M-MLV RT comprising amino acid substitutions D200N, T330P, L603W, T306K, and W313F as compared to the wild type M-MMLV RT as set forth in SEQ ID NO: 4448. In some embodiments, a split prime editor comprising the D200N, T330P, L603W, T306K, and W313F as compared to the wild type M-MMLV RT may be referred to as a “PE2” split prime editor, and the corresponding prime editing system a PE2 prime editing system.

Exemplary wild type moloney murine leukemia virus reverse transcriptase:

(SEQ ID NO: 4448)
TLNIEDEYRLHETSKEPDVSLGSTWLSDFPQAWAETGGMGLAVRQAPLIIPLKAT
STPVSIKQYPMSQEARLGIKPHIQRLLDQGILVPCQSPWNTPLLPVKKPGTNDYRPVQDL
REVNKRVEDIHPTVPNPYNLLSGLPPSHQWYTVLDLKDAFFCLRLHPTSQPLFAFEWRD
PEMGISGQLTWTRLPQGFKNSPTLFDEALHRDLADFRIQHPDLILLQYVDDLLLAATSEL
DCQQGTRALLQTLGNLGYRASAKKAQICQKQVKYLGYLLKEGQRWLTEARKETVMGQ
PTPKTPRQLREFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLDPVAAGWPP
CLRMVAAIAVLTKDAGKLTMGQPLVILAPHAVEALVKQPPDRWLSNARMTHYQALLL
DTDRVQFGPVVALNPATLLPLPEEGLQHNCLDILAEAHGTRPDLTDQPLPDADHTWYTD
GSSLLQEGQRKAGAAVTTETEVIWAKALPAGTSAQRAELIALTQALKMAEGKKLNVYT
DSRYAFATAHIHGEIYRRRGLLTSEGKEIKNKDEILALLKALFLPKRLSIIHCPGHQKGHS
AEARGNRMADQAARKAAITETPDTSTLLIENSSP.

In some embodiments, an RT variant may be a functional fragment of a reference RT that have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., a wild type RT. In some embodiments, the RT variant comprises a fragment of a reference RT, e.g., a wild type RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the reference RT. In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a corresponding wild type RT (M-MLV reverse transcriptase) (e.g., SEQ ID NO: 4448).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end compared to a reference RT, e.g., a wild type RT. In some embodiments, the reference RT is a wild type M-MLV RT. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end compared to a reference RT, e.g., a wild type RT. In some embodiments, the reference RT is a wild type M-MLV RT. In still other embodiments, the RT truncated variant has a truncation at the N-terminal and the C-terminal end compared to a reference RT, e.g., a wild type RT. In some embodiments, the N-terminal truncation and the C-terminal truncation are of the same length. In some embodiments, the N-terminal truncation and the C-terminal truncation are of different lengths.

For example, the split prime editors disclosed herein may include a functional variant of a wild type M-MLV reverse transcriptase. In some embodiments, the split prime editor comprises a functional variant of a wild type M-MLV RT, wherein the functional variant of M-MLV RT is truncated after amino acid position 502 compared to a wild type M-MLV RT as set forth in SEQ ID NO: 4448. In some embodiments, the functional variant of M-MLV RT further comprises a D200X, T306X, W313X, and/or T330X amino acid substitution compared to compared to a wild type M-MLV RT as set forth in SEQ ID NO: 4448, wherein X is any amino acid other than the original amino acid. In some embodiments, the functional variant of M-MLV RT further comprises a D200N, T306K, W313F, and/or T330P amino acid substitution compared to compared to a wild type M-MLV RT as set forth in SEQ ID NO: 4448, wherein X is any amino acid other than the original amino acid. A DNA sequence encoding a split prime editor comprising this truncated RT is 522 bp smaller than PE2, and therefore makes its potentially useful for applications where delivery of the DNA sequence is challenging due to its size (i.e., adeno-associated virus and lentivirus delivery). In some embodiments, a split prime editor comprises a M-MLV RT variant, wherein the M-MLV RT consists of the following amino acid sequence:

(SEQ ID NO: 8001)
TLNIEDEYRLHETSKEPDVSLGSTWLSDFPQAWAETGGMGLAVRQAPLIIPLKATSTPVSI
KQYPMSQEARLGIKPHIQRLLDQGILVPCQSPWNTPLLPVKKPGTNDYRPVQDLREVNK
RVEDIHPTVPNPYNLLSGLPPSHQWYTVLDLKDAFFCLRLHPTSQPLFAFEWRDPEMGIS
GQLTWTRLPQGFKNSPTLFNEALHRDLADFRIQHPDLILLQYVDDLLLAATSELDCQQG
TRALLQTLGNLGYRASAKKAQICQKQVKYLGYLLKEGQRWLTEARKETVMGQPTPKT
PRQLREFLGKAGFCRLFIPGFAEMAAPLYPLTKPGTLFNWGPDQQKAYQEIKQALLTAP
ALGLPDLTKPFELFVDEKQGYAKGVLTQKLGPWRRPVAYLSKKLDPVAAGWPPCLRM
VAAIAVLTKDAGKLTMGQPLVILAPHAVEALVKQPPDRWLSNARMTHYQALLLDTDR
VQFGPVVALNPATLLPLPEEGLQHNCLDNSRLIN.

In some embodiments, a split prime editor comprises a eukaryotic RT, for example, a yeast, drosophila, rodent, or primate RT. In some embodiments, the split prime editor comprises a Group II intron RT, for example, a. Geobacillus stearothermophilus Group II Intron (GsI-IIC) RT or a Eubacterium rectale group II intron (Eu.re.I2) RT. In some embodiments, the split prime editor comprises a retron RT.

In some embodiments, the RT comprises an amino acid sequence having at least 90% (e.g., at least 91%, at least 92%, at least 93%, at least 94%, at least 95%, at least 96%, at least 97%, at least 98%, at least 99%, or at least 99.9%) sequence identity to any one sequence as set forth in Table 11, 12, or 13. In some embodiments, the RT comprises any one sequence as set forth in Table 11, 12, and Table 13.

In some embodiments, the DNA polymerase domain comprises any one of the sequences in Tables 11, 12 or 13.

TABLE 11
Exemplary RT Homolog (RT domain) Sequences

| SEQ | | | |
|---|---|---|---|
| ID | | Accession | |
| NO: | Amino acid Sequence | Number | Species |
| 8021 | HAQLLRSIKARFPDCTRKSVVRSGDESPLRTPGK | AAG17765 | Rhodomonas |
| | FEKAWRAKVTTRRLTKLIHNGCIILFGVRYHPG | | salina |
| | KGTSTSWSPFRIYWRPIATGVSRSNQDTFTSVDI | | |
| | MQRAKVTYFGGCGNMRYPTARKSHGYGASIIG | | |
| | FTRREAGLRLYTQNSAISDSSMTSCGIISKHRKF | | |
| | NKDKNFVNKRLINIIGDVQTLIVAYEFVKSKPGQ | | |
| | MVKGSIDSTLDDIDLAWIKSISKVIKAGKFKFIPS | | |
| | RRIYVSKTGCKERRPIMTGFPRDKLVQKAIQLV | | |
| | LEPIYENVFLENSHGFRPARGCHTALKSIKQGFH | | |
| | GVTWVIESDIASCFSSVNHEVLLSIIKERIKCVKT | | |
| | LALIRNLLESGYVDLGAFCKSKLGIPQGSSLSPL | | |
| | LCNIYLHKFDTFMYELKQRFVYTSSKDPRINPA | | |
| | YKRLQRQIQNTPGLVEKSKFIQELRKTPSKDLFD | | |
| | PKYRRLFYIRYADDFSIGITGQKKDAVEILDQAK | | |
| | IFLSEELKMDLKESKIRVVHLKKQSIFFLGTTIYG | | |
| | ISCVEKPMRTVKHSNWKTSIKIRVTPRVGLHAP | | |
| | MKVLLEKLLQNKFVKRDKEGIFKPTALGKLVN | | |
| | FDHADIIGYYNSVARGIMNYYSFVDNYSRLGSI | | |
| | VKYYLLHSCALTLALKYKLRFKSKAFKRFGGK | | |
| | LKCPDTKKEFFIPKNFFRTEKFSINPPDTEQVISK | | |
| | RWNNKLTKSSLFKACVICGTTPAEMYHVCKTR | | |
| | DLRNKYKTKKLDFFKFQMASFNQKQVPLCQFH | | |
| | HKSLHQGKLSEADKVAFREGITNL | | |
| 8022 | LPDTIERAVRSLPTVIRSGRKVNGLYRLLKSPLL | AAD03884 | Novosphingobium |
| | WEHAYQRIAPNKGAMTPGVDGQTFDGFSPDKV | | aromaticivorans |
| | RSIIERLANGTYRPQPARRVYIPKANGQKRPLGV | | |
| | PTTEDKLVQEVVRTILEQIYEPLFSRHSHGFRPK | | |
| | RSCHTALESIRAIWTGVKWLIDVDVVGFFDNID | | |
| | HDVLVSLLEKRIADRRFVRLIRGLLKAGYVEDW | | |
| | VFHKTYSGTPQGGVVSPMLANIYLHELDMFMQ | | |
| | AKMAGFDKGKQRSPSPDARRIRNRLSYVRRTV | | |
| | DQLRAKGRGDDPRVTSFLEEIGRLKAERLAVPA | | |
| | SDAFDPNYRRLRYCRYADDFIIGVTGSKSEARQI | | |
| | MEEVRTYLSDHLKLAVSAEKSGIHKASDGARFL | | |
| | GYEVRTMTNPNPHKAIFDGRPAVRRGLADRMK | | |
| | LLVPRDRVVRFVNSKEWGDYDSFRPVGRAALR | | |
| | FASDVEIVLAYNAEWRGFANYYAIADDVKRKL | | |
| | NKAGYFALLSCVKTIAGKHRTSARRVFAKLRR | | |
| | GTDFYISYEVGDTTRTIKLWQLKDLQRHTRTW | | |
| | GGIDIPSSAKFVFSRTELVERLNARECERCGSND | | |
| | QPCEVHHVRRIGELQHAGFSRHMAAARQRKRM | | |
| | VLCSRCHNDVHAGQPTDRQRRTARSRGEPNAL | | |
| | KGARSVRRGA | | |
| 8023 | ASKETGMFSLAGELASLVEESSSHVDDDSKPRS | NP_177575 | Arabidopsis |
| | RMELKRSLELRLKKRVKEQCINGKFSDLLKKVI | | thaliana |
| | ARPETLRDAYDCIRLNSNVSITERNGSVAFDSIA | | |
| | EELSSGVFDVASNTFSIVARDKTKEVLVLPSVAL | | |
| | KVVQEAIRIVLEVVFSPHFSKISHSCRSGRGRAS | | |
| | ALKYINNNISRSDWCFTLSLNKKLDVSVFENLLS | | |
| | VMEEKVEDSSLSILLRSMFEARVLNLEFGGFPK | | |
| | GHGLPQEGVLSRVLMNIYLDRFDHEFYRISMRH | | |
| | EALGLDSKTDEDSPGSKLRSWFRRQAGEQGLKS | | |
| | TTEQDVALRVYCCRFMDEIYFSVSGPKKVASDI | | |
| | RSEAIGFLRNSLHLDITDETDPSPCEATSGLRVL | | |
| | GTLVRKNVRESPTVKAVHKLKEKVRLFALQKE | | |
| | EAWTLGTVRIGKKWLGHGLKKVKESEIKGLAD | | |
| | SNSTLSQISCHRKAGMETDHWYKILLRIWMEDV | | |
| | LRTSADRSEEFVLSKHVVEPTVPQELRDAFYKF | | |
| | QNAAAAYVSSETANLEALLPCPQSHDRPVFFGD | | |
| | VVAPTNAIGRRLYRYGLITAKGYARSNSMLILL | | |
| | DTAQIIDWYSGLVRRWVIWYEGCSNFDEIKALI | | |
| | DNQIRMSCIRTLAAKYRIHENEIEKRLDLELSTIP | | |
| | SAEDIEQEIQHEKLDSPAFDRDEHLTYGLSNSGL | | |
| | CLLSLARLVSESRPCNCFVIGCSMAAPAVYTLH | | |
| | AMERQKFPGWKTGFSVCIPSSLNGRRIGLCKQH | | |
| | LKDLYIGQISLQAVDFGAWR | | |
| 8024 | NTSERASAHVSYTNWKAVQMYVTKLRQRIYRA | NP_832403 | Bacillus cereus |
| | EQLQQQRKVRKLQRLLMRSEANLLLSIRRVTQQ | | |
| | NKGKRTAGVDEHTALSRRERNLLYEQLKKLNT | | |
| | LQHRPKPAKRIYIVKKNGKLRPLGIPTIKDRVYQ | | |
| | NIVRNALEPQWEARFEAISYGFRPKRSTHDAIRS | | |
| | IFNRINGGTKKKWIVEGDFQGCFDHLNHEWILK | | |
| | QTSYFPGRKLLKRWLKMGYMEQSFFAETQEGT | | |
| | PQGGIISPLLANIALHGMEETLGITYKKNYKAND | | |
| | SYIMNPACKFTLIRYADDFVVLTETKEQALSVY | | |
| | MRLRPYLKDRGLELSPEKTKVTHIEEGFEFLGFL | | |
| | IRQYQTEQGNKLFIKPSKGSRQKAKKKIGDTLR | | |
| | VMRGQPIGEIIRVLNPIIRGYGQYWKHVVSKKIF | | |
| | GTMDSYIYWRIGKHLRQLHPKKSWKWIYARYY | | |
| | RHPHHGGNAWTPTCPKTNIQLLHMSWIKIERHN | | |
| | MVKFKNSPDDPTLKEYWEKRDRKVFDTENTM | | |
| | DRMKLARKQGYRCAICKTPLQNGEKVVVKDM | | |
| | PVPQHLILSNLNLKLVHLPCLY | | |
| 8025 | DSKDMQRLQTTQQRGYPLDREMEFQKTTEVHS | ZP_00778259 | Thermoanaerobacter |
| | ISSASRDGRNEVQRYTSKMLEMIVERGNMRAA | | ethanolicus |
| | YKRVVANKGSHGVDGMEVDELLPYLKENWPTI | | |
| | KQQLLEGKYKPQPVRRVEIPKPDGGVRLLGIPT | | |
| | ALDRLIQQAIAQILNRVYNHTFSDSSYGFRPGRS | | |
| | AKDAIKAAEAYINEGYTWVVDMDLEKFFDRVN | | |
| | HDIIMSKLEKRIGDKRVLKLIRRYLESGVMINGV | | |
| | KVSTEEGTPQGGPLSPLLANIMLDELDKELEKR | | |
| | GHKFCRYADDCNIYVKSRSAGNRVMKSIKKFIE | | |
| | SKLKLKVNEAKSAVDRPWRRKFLGFSFYTKEN | | |
| | EVRIRIHEKSIKRFKEKVREITNRNKGISMENRIK | | |
| | RLNQITTGWVNYFGLADAKSIMKTLDEWIRRRL | | |
| | RACIWKQWKKIKTKHDNLVKLGVEEQKAWEY | | |
| | ANTRKGYWRISNSPILNKTLTNKYFESIGYKSLS | | |
| | QRYLIVHNS | | |
| 8026 | QETKPFGISKNVVMMAFERVKANKGTYGMDE | ZP_00738538 | Bacillus |
| | QSIEMYEMDLKNNLYKLWNRMSSGSYFPKPVK | | thuringiensissero |
| | AVAIPKKNGGTRTLGIPTVEDRVAQMVAKLYFE | | varisraelensis |
| | PNVERLFYEDSYGYRPNKSAIQAIEATRKRCWR | | |
| | KDWVLEFDIKGLFDNIRHDYLIEMVKRHTNQE | | |
| | WVTLYVQRWLITPFQMEDGTLIERTAGTPQGG | | |
| | VISPVLANLFLHYTFDDFMVKEFSSIPWARYAD | | |
| | DGIAHCTSLKQAKYLQRRLEERFKLFGLELNLE | | |
| | KTKIAYCKDDDRQLSYPNTSFDFLGYTFRPRHA | | |
| | KNKHGKFFTNFSPAIADKAKKAIRKEVRSWRLQ | | |
| | LKADKTLQDISNMFNKKIQGWINYYGHFYKSE | | |
| | MYSVLRYINSSLIKWVRRKYKKRKHRRKAEYW | | |
| | LGTIAQRERKLFAHWKYGILPATNNGSRMS | | |
| 8027 | KKEKIDGWYKSGRNYLHFDEKVSFEKASRIVK | ZP_01457859 | Desulfovibrio |
| | NPRKVASWNFFPFLQTTVKTSKITRNDEGEIVPK | | vulgaris DP4 |
| | NKSRPISYAAHTDSHIYSYYATLLQPIYEKFIEKH | | |
| | GLGTNITGFRKLDGECNIDFAHRAFNAIRSMTPC | | |
| | IALSFDVKSFFDEIDHSILKQAWCTILEKTLLPED | | |
| | HFAIFKSLTTYSYVDRDDAFNAFGITKSSKKNGI | | |
| | RRICNPLEFRSILRPAGLIKRNKNSYGIPQGSPISG | | |
| | LLSNIYLFEFDKAISYFASDTKSHYYRYCDDIIIIC | | |
| | NEEHEELFKNLVSDELKKLNLRTNEKNVIRKFF | | |
| | MGCDGPECDKPIQYLGFVFDGKRAVIRSASHSR | | |
| | FLKRMRKAVSLAKQTKRKRDKIRTSKGLETTSL | | |
| | HKKKLYTKYSYLGNRNYISYAHRAAKIMDEDA | | |
| | IKKQVKPLWKRLRQEIESD | | |
| 8028 | SMKEFALNLSALYSAFDAVKENHGCAGADGVT | CAJ74578 | Candidatus |
| | IERYEGNLDLNLRIMRKELTEQTYFPLPLLRILV | | Kuenenia |
| | DKGNGEARALCIPSVRDRIVQAAVLQLIEPVLE | | stuttgartiensis |
| | KEFEECSFAYRKGRSVKQAVYKVREYYEQGYQ | | |
| | WVVDADIDAFFDSVDYSLLLLKFKCYIHDPCIQ | | |
| | NLVGLWLKGEVWDGKTVTTLKKGIPQGSPISPI | | |
| | LANLYLDEFDEELTRNGYKLVRFSDDFIILCKNS | | |
| | GMAKESLKLTKKILEKLLLELDEEQVINFDQGF | | |
| | KFLGVIFVKSMIMVPFDRPKKERKVLFFPKPLDL | | |
| | EVYFKQRKQGKIWQTST | | |
| 8029 | KKYSLTPAMLEVCKEYNFFSDVSGFMPLTSENI | ZP_00813439 | Shewanella |
| | KEIKIGKKFAYSHQDNNKPTHQKLSKIIFENFLS | | putrefaciens |
| | NIPLNQSAIAYVKKKSYFDFIEPHRNNYFFLRIDL | | CN-32 |
| | KDFFHSISEDLLKRTLSDYFSSESLSETIKQSNID | | |
| | AIFTFLTVNLKSDSSNVKFLDKKILPIGFPLSPNL | | |
| | ANIVFRKTDLLLEKLCDMHGVTYTRYADDMLF | | |
| | SSRGIMEKNLLFRKNNNYKKPYIHSDNFLSEIKY | | |
| | LVSIDGFFINHNKTIKSVNTLSLNGYTISGTNFPD | | |
| | JEGKIRLSNKKTKIIEKVIHEININPDDKVTFEKCF | | |
| | KKEFPKPKYEKNRDNFINNLCTIKINQKLLGYRS | | |
| | YLISIIKFNNKFNCISESSEDKYNTLLSKIENVIKK | | |
| | RIK | | |
| 8030 | EHTYYPHHPINSLKALNRALGIDEDEIFHALSNIS | YP_573256 | Chromohalobacter |
| | YKEVPIKKKDGAIRVTYDATSALKVVQKKVTS | | salexigens |
| | NIFHRVNFPHYIHGCIRDTKTPRNIYTNAYPHAG | | DSM3043 |
| | CKQVILCDIKDFFPSIKAKTVFFIFRHCLGFSPNV | | |
| | SQRLTDLCTYNGTLPQGASTSSYIANLAFWDVE | | |
| | PLLVKKLESLGLTYTRFADDITISAKKSISKSLKT | | |
| | QVLHEVRRTIRKRGCSLKKNKTIVLKRSQVIIGK | | |
| | DKETQETTRNPITVTSLSIHHSSVTISKVERRKVR | | |
| | AFVDKLSKTEFNRVSYHEWCRRYSSAMGRVSR | | |
| | LISCGHKEGEPLKQRLKALKDEHKKFHQRSSNV | | |
| | SGRK | | |
| 8031 | HTVHGNFFYKLSNKKRLAKYLRVSLKELLTLQ | YP_797537 | Leptospira |
| | DDSNYKVWSEEGENGKLREIQEPVYKLKSVHS | | borgpetersenii |
| | KIQKSLASIVAPEFLFSGVKGKSNISNASYHKDG | | |
| | NYIVTADIQSFYINCNKEHIFRFFKYTLRTSDDIA | | |
| | RILTELCCYKTFLPTGSPVSQILAYHSYARIFNRI | | |
| | DAFSKANDITFSLYVDDITLSSEKSIHRYILKTIS | | |
| | KLLKSVNLSLKKEKTKFYNKNSYKIVTGCAISP | | |
| | DHILLRPNKIMRKIDNKLCKHEKDLSKLTPKEIE | | |
| | SVLGQVIYLRSITPNSYPQLFKSLSLMKTEEKIK | | |
| | QRPL | | |
| 8032 | DKKAIYIERFLVYAPRKYRVYKIPKRKHGSRVIA | YP_856565 | Aeromonas |
| | QPTAELKKLQRAFINRSKIPVHECAMAYKDGVS | | hydrophila |
| | IKDNAQLHSTNTFFLSMDFENFFNSITPDLLWGV | | ATCC7966 |
| | FNKFGKVISPNEKLWLSKLLFWCPSKKNSNKLI | | |
| | LSVGAPSSPKVSNFCMYFFDEYISTYCQDRNITY | | |
| | SRYADDLSFSTNEKDILFQIPGVVKETLLKLFGR | | |
| | DITINNSKTVFSSKAHNRHVTGITITNEGELSLGR | | |
| | EKKRYIKHLIFRFKNGLLDVSDVSYLRGILSFAF | | |
| | YIEPAFKTSMVKKYTKATIDSIFNGVDDGK | | |
| 8033 | KILKIPKKNGKYRTIYAPDAEEKRALRGIVGILN | GAA02480 | Pelotomaculum |
| | QKCQHVCDPAAVHGFMPLKSPVTNALAHVGR | | thermopropionicum |
| | KYTVSFDLEDFFDTVTPEKASKCLTKEQKELVF | | |
| | VDGAARQGLPTSPAVANLAATDMDRAILKWIE | | |
| | KSGKSVVYTRYADDLAFSFDDPELIPVIQKKVPE | | |
| | IIRRSGFRVNTDKTTVQAAVAGRRIICGVAVDD | | |
| | EGVHPTREVKRRLRAAKHQGNELEAAGLEEWC | | |
| | KLKVPSGKRQKARETTEGLDELRKHWKLRKID | | |
| | MAKAVSRKVIPEKDLGDNCYITNDPAYFMGMS | | |
| | TFTTGWKSCMRMDGGEYRKGVMAWLALPGTS | | |
| | VAVFLSDRTMNIAGVERRRMRARCLVHKLENG | | |
| | QLVYDRLYGNPDDTPVLVKKLEEAGIRPIREFA | | |
| | GKGIYVEGDVPASMAMPYCDNLWEEKINIKSG | | |
| | KRVVRFYV | | |
| 8034 | SITDLALAIGVSPRLITSFIHAPGNHYRHFNIGKR | EAT03589 | Deltaproteobacterium |
| | GGGERVISSPRTFLKVVQYWILDYLLHPLPCHP | | MLMS-1 |
| | NCHSYQKGKSILSNSLPHVGKKYVANIDILNFFP | | |
| | SITERMVFDFLKKNNFGEQLSKSLSRIVTLNNGL | | |
| | PQGAPTSPVISNSFLNKFDEIISEKSLLLDVSFTR | | |
| | YADDITISGDRKENIISLIEISEHYLNSIGLKLNNK | | |
| | KTRIASKGGQQRVTGIVVNKTAQPPRKFRKNIR | | |
| | SMFHHAGMKPELFVDKINVLRGYVSYLQSFPNL | | |
| | YDGNEIKKYKKICATIQANFVQKQ | | |
| 8035 | EWIEYREQFITTAKKASKNSGYIKKNLKYAEKL | NP_603068 | Fusobacterium |
| | YNQKLPIIYNASHFSKLVGYSLQYLYAASNDSS | | nucleatum |
| | KFYREFEIKKKNGGSRKISEPLPSLKEIQKWILEN | | ATCC25586 |
| | ILNKIQKEKVSKYAKAYQKKISIKENVKFHRGQ | | |
| | KKVLSLDITNFFLNIKIDKIYEVFYNLGYSKSLST | | |
| | LFSNLCTLNYSLPQGAPTSPILSNIVMLNFDNEIE | | |
| | KIVLEKRIRYTRYADDMTFSGDFLEKEIIKYVKE | | |
| | NLNKIGLKINNKKTRVRKNWQQQMVTGIIVNE | | |
| | KIQISRKKRDELRQTMYYIEKYGIDSFLKYKGIK | | |
| | NKVYYLSHLKGILEYAYFINKNDKKLFNYIEYL | | |
| | KNNFFKEKSSI | | |
| 8036 | TIEVQRWEDKFEIKPGVWVYVPSVEARKVGGKI | NP_806368 | Salmonella |
| | LQAVRNKWIPPLYFYHLRTGGHLKAARLHLKS | | enterica |
| | DFFAVVDIKQFFQSTSRSRITRDLKSYFTYSQAR | | |
| | EISTFSTVRNLSHSPHKHVLPFGFVQSPILATLCL | | |
| | DKSYFGSLLRRLNKHHDLKLSVFMDDVIISSNN | | |
| | LAQLQAAYDEALVAMRKSGYQANMSKTQAPS | | |
| | SKISVFNLTLSKGVMKVTSQKMSDFLIDFYSSN | | |
| | YEPHRIGVKNYVEAVNPGQAKLFKL | | |
| 8037 | NRWSSRAFKKHNSDKPAAVVETAALYGRKIQT | ZP_01043439 | Idiomarina |
| | SCPELPVIFTLNHLALKSGVPYNNLRSFVDRTIE | | baltica OS145 |
| | RPYRSFTLRKNGLGSNPRKFRVIKVPQQDLKKA | | |
| | QQFINQNILSKMEPHECSVAFSPGSKIYDAASEH | | |
| | CNARWLLKFDIVSFFESITEKSVYRVFRRYNYPA | | |
| | LLSFEMARICTALKARPPINWGGAPPNIRYRTIP | | |
| | GYSNKNLGTLPQGAPTSPMLANLVSYNLDRRL | | |
| | KLIADAYNCHYSRYADDITFSTDSSLSRGEVSRII | | |
| | AMINSTLREYGHTMNKAKTTIAPPGARKFYLG | | |
| | MNIHGDSPQLRKSFKRKLKQHLFFCEKNSVGPE | | |
| | KHSKHLGFVSVIGFKNHLRGLINYANQVDTVFG | | |
| | ENCMKRFQSIDWPL | | |
| 8038 | EKIENEIVNKTYLAINSLEELRNMIGIKSDYFYK | BAB43301 | Staphylococcus |
| | CLYVNDHFYNVIKIPKRKKDEYRELMIPNMALK | | aureus N315 |
| | NIQRWILDNVLYRRQVHKCATGFVPRKSIVNNA | | |
| | IPHVGQKYILKMDIENFFPSITFKQVRKIFSEMGY | | |
| | KFELATALANLCTVNNQLPQGAPTSPYIANIIFY | | |
| | NIDKRIFSYCQKNNLRYTRYADDITISGSNKVSF | | |
| | SKEIIREIVNQYNFRINESKTIMFKPGDRKKVTGI | | |
| | IVNEKISVPKTLIREVRKQIYFVNKFGLEEHLIRN | | |
| | NYSLDYEQQFIMSIYGKISFIKMIDFKKGVSLQK | | |
| | KFNEVLGNIESSNMYRDNIDFDDIELHWIN | | |
| 8039 | TARLDPFVPAASPQAVPTPELTAPSSDAAAKRE | P23072 | Myxococcus |
| | ARRLAHEALLVRAKAIDEAGGADDWVQAQLV | | xanthus |
| | SKGLAVEDLDESSASEKDKKAWKEKKKAEATE | | |
| | RRALKRQAHEAWKATHVGHLGAGVHWAEDR | | |
| | LADAFDVPHREERARANGLTELDSAEALAKAL | | |
| | GLSVSKLRWFAFHREVDTATHYVSWTIPKRDG | | |
| | SKRTITSPKPELKAAQRWVLSNVVERLPVHGAA | | |
| | HGFVAGRSILTNALAHQGADVVVKVDLKDFFP | | |
| | SVTWRRVKGLLRKGGLREGTSTLLSLLSTEAPR | | |
| | EAVQFRGKLLHVAKGPRALPQGAPTSPGITNAL | | |
| | CLKLDKRLSALAKRLGFTYTRYADDLTFSWTK | | |
| | AKQPKPRRTQRPPVAVLLSRVQEVVEAEGFRV | | |
| | HPDKTRVARKGTRQRVTGLVVNAAGKDAPAA | | |
| | RVPRDVVRQLRAAIHNRKKGKPGREGESLEQL | | |
| | KGMAAFIHMTDPAKGRAFLAQLTELESTASAAP | | |
| | QAE | | |
| 8040 | YSQNLTTIPLATESNLERLETDNLALLRSHGLAE | ZP_00112324 | Nostoc |
| | YNTAEEIAFAMVISLEKLHFLTTSTSLTRHYLPF | | punctiforme |
| | KISKKTGGKRIISAPKPELKAAQRWILENILEKLE | | PCC73102 |
| | VHNAAHGFCKNRSIVTNAKPHVGANVIVNIDLQ | | |
| | NFFQSISYKRIKELFSGFGYSESTATIFGLICTTAE | | |
| | IAINGQINHTASENRHLPQGSPASPAISNLVCRNL | | |
| | DIRLAAIAENLGFCYTRYADDLTFSTSEDASSKI | | |
| | SNLIKNTKFIIHGENFTVNDNKTKISSKSVQQEV | | |
| | TGVIVNTQLNISKKTLKAFRATLYQIEQEGLSGK | | |
| | SWGKSTNLIAAITGFANYVAMINPDKGAEFKSS | | |
| | VERIKQKYGGSQTDEVRF | | |
| 8041 | AGMPGFVSAWRSEQPPRVVRVLTRPPFQRPPPP | ZP_01511780 | Burkholderia |
| | WLHDVALPQLPTLGDLAAWLDIEPGDLGWFAD | | phytofirmans |
| | RWRVPTRGAATPLHHYAYKAIEKRDGRCRIIEIP | | |
| | KPRLRALQRKVLSGLLDRIPAHESVHGFRHGRN | | |
| | IVTFAAPHVGKAVVMRFDLTDFFASVHAGRVY | | |
| | SAFYALGYPQAVARALTALCTNRIPSGRLLAPD | | |
| | VRERIDWRERQRYRNRHLPQGAPTSPALANLC | | |
| | AFRLDLRLAGLARSVGATYTRYADDLAFSGDE | | |
| | ELARMADRLCIRVAAIALEEGFGVNLRKTRVM | | |
| | RRSARQHLAGVVVNSHANVARPEFDALKAVLT | | |
| | NCVRHGWRSQNRDDLADFRAHLAGRVAHVA | | |
| | MVNAVRGARLRAVFERIEWEEEKPLDA | | |
| 8042 | ELLLGIVIVVTCWMVVRIIRSSKNQEGYKRWRA | BAD47792 | Bacteroides |
| | GNYASENPYAKEKASGPLSQGLFSKRVRTTGAR | | fragilis YCH46 |
| | RFDDGAIRWCANLLATEESRLREVLDYIPRQYT | | |
| | CFHVRKRSGGFRYISAPAGDFRSMQQTIYHRILL | | |
| | LANIHPAVTGFCPGKSVSDNARVHLGRKNVLK | | |
| | VDLHDFFPSIRSPRVRAAFREMGYSRPIAKVLAE | | |
| | LCCLRCCLPQGAPTSPALSNIIAYPMDKKMMAL | | |
| | AGEYGLVYTRYADDLTFSGDYLPKDEVLVRIH | | |
| | RIIREEGFTMNVKKTRFLSEHKRKIITGVSVSSG | | |
| | KKMTLPKVKKREIRKNVHYVLTKGLVGHQEHI | | |
| | GSTDPVYLKRLLGSLCYWRSIEPDNRYVSDSITA | | |
| | LKRLM | | |
| 8043 | KTLKNIEDRKDLADYLNIPIKRLTYILYIKRTENL | ZP_00231674 | Listeria |
| | YYSFEIPKKSGGVRNIDAPKSELKALQKKLAAS | | monocytogenes |
| | LTKYQEILQKSKRKAPNISHGFEKGKSIISNAKIH | | 4bH7858 |
| | RNKKIVYNLDLENFFESFHFGRVRGFFEKNKDF | | |
| | ELSTEVATIIAQLSCFNGALPQGAPSSPIITNFICRI | | |
| | MDMRILKLAKNYKLDYTRYADDLTFSTNDKKF | | |
| | IDQIDYFLHKLTKEIEKAGFKLNKNKTNLNFKDS | | |
| | RQLVTGLVVNKKINVDRRYYKETRAMAHRLY | | |
| | KTGEFQIDDKNGTLNQLEGRFSFINQVQRYNNV | | |
| | IDSSKHDFNNLNAFEKQYQAFLFYKYFYANNKP | | |
| | HIVTEGKTDINYIKAALKKHHLEFPNLIVKKEDG | | |
| | EFDFRVAFLKRTNRLAYFLNIKKDGADTMKNIC | | |
| | KYFFDIENNEVPNYLKTFKILTKQIASNPTILIFD | | |
| | NEISNNVKPVSKIIKYIKLKEDSRVMLTEKSYLN | | |
| | LEDSLYLLMNPLVKNKKECEIEDLFDEATLNHEI | | |
| | NGKKFSREKNMDLNKYYSKERFSNFIYNEYREI | | |
| | DFSNFKPMLENLNFIIENYKNEK | | |
| 8044 | DHFSSVVDNYILQTNKDGHYCWRPFELIHPAIY | YP_670592 | Escherichia coli |
| | VHLVHKITEDESWQLLLERFGEFQSNTKIVCASL | | 536 |
| | PRESEEDGVSDKAKAVSGWWRDVEQESINKSL | | |
| | QFKYLFSTDIANFYPSIYTHSIPWAIYTKEDAKA | | |
| | ARGAGRNLGDQIDYALRQMRWGQTNGIPQGSA | | |
| | LMDFIAEIVLGYADELLGQKLESQNINDYHIIRY | | |
| | RDDYRIFTNSKEDAEAIARHLTVILQGLGLQLN | | |
| | ASKTSLTEDLVLGSMKPDKQEALMVFGRSVNA | | |
| | TTIQKTLLKLVIFSRKYKNSGQLEAYLAKINKRL | | |
| | ERMSSIKEEVRAIVSIISDLMINNPRTFSSCALVL | | |
| | SNVLKFVDNDVEKLELLKQIKDKFKPILGTGILD | | |
| | IWLQRISYHINRDIEYKETLCSIVSGVHDRPHEH | | |
| | VWNSEWISDNNFKNSIMATSFVDNDKLQACEPI | | |
| | IPEEEVTLFPYDSDVDEEDLE | | |
| 8045 | KDLTAKDLIGKGYFPKEIPKGFSTNSLAEKFSNL | ZP_01171347 | Bacillus |
| | DFSTFTKKERGKWYKTSNISIPKFAHSRRILNVP | | NRRLB-14911 |
| | APFPQMRLSQLLVKNTEELNEYYSQSKLSLTRPI | | |
| | VKEESDRAVERKYHFSKIIERRIESINDKKYILKT | | |
| | DISRYFPTIYTHSIPWALHTKEVAKQTRGDSLLG | | |
| | NTIDEYVRNIQDGQTMGLPVGPDTSLIISEVIGT | | |
| | AIDIKLQEAHPNIIGSRYTDDFEFYFKTQSEAEK | | |
| | VLNTIQEIVRHFELDINPVKTEIISSPNLLEPIWLS | | |
| | NLKLYQFRSSATAQKNDIKTFFSTAFYYQNQSP | | |
| | YEGVLKYCLKKIKNLKIKEDNWSLFEALILHIM | | |
| | LIDPTTLPLIENILFGYKEIGYPINEQKIKDTIAEIF | | |
| | ASNIAVGNNYEIMWALSLSNKLQLKISNESSKL | | |
| | LFNLEDSFSNILTMEAYTNGYIEGGYEPEYFKTL | | |
| | LNENELYGRNWLFAYEMSVKGWLKPHQQKEY | | |
| | VKKDTFYNQLFESKVEFYHNDRKAEIKNDDWL | | |
| | TALLNDDDIEELFIQNASSKKPYLRGGSGGADY | | |
| 8046 | KASKLRPRDFLQCLLSTAYLPEELPPTVTSREYS | NP_766843 | Bradyrhizobium |
| | EFCRRNYALVRAEKDKLIKLATSYDTYSAPRNV | | diazoefficiens |
| | PGRRALAVVHPLAQLGVSLLITERRAEIRSLLKK | | USDA110 |
| | SGTSLYDVSEAAAQAKAFAGLDFQKRRTLAAK | | |
| | LHSEKPFILQADISRFFYTAYTHSIPWAVLGKEK | | |
| | AKELLRTNRKKLNAHWSNKIDEALQSCQSRETF | | |
| | GIPVGPDTSRVLAELLLSGVETDKSLSKYLQPTN | | |
| | AFRLLDDFSIGFDNEADARQALRAIRQVLWRYN | | |
| | LQLNEEKTKIITSPLIFREKWKLDFDKAPLSQIDP | | |
| | QQQLRDIEYLVDLALNACFESRGNSGSMGLPPP | | |
| | KSGDSSRRHLSHLA | | |
| 8047 | APDKDFVLIALLKYNYFPAHKKEKEELPPIFSTK | CAJ75424 | Candidatus |
| | QFTKKIAQKLSHLRSRKDGYDQISYKITRYNNIP | | Kuenenia |
| | RVLSIPHPKPYADIVFCLSENWDNLAYICNNEVS | | stuttgartiensis |
| | LIRPRQHKDGRIIIMNYEGSHEKIERSLKKSFGH | | |
| | KFCIETDITNCFPSIYSHAIPWALIGLKEAKSRKR | | |
| | YKNEWFNKIDARQRMLKRNETQGVPIGPASSNI | | |
| | ITEIILAKVDEVMSKDFNYIRFIDDYTCYCKKYE | | |
| | DAEEFARRLSQELSKYNLTLNLKKTHIHQLPKP | | |
| | TNDDWIIDLKSRISGDSKKITHYQAVNYIDYAVS | | |
| | LNKKIPDGSILKYAFKSIRGRLNVKDERFCLNYI | | |
| | LLLAPHYPILIPIINKMLHNASKKDKFVYEKELK | | |
| | YILDESIVNHRSDGMCWTLYYLLKNKVTLSPEI | | |
| | AKHIIATKDCMAILMLYLFKKFDTEINGFANGL | | |
| | DKTDLYGLDNYWLLLYQLFYDNKIKNPYKDEE | | |
| | TFKILKDNNVNFIQSK | | |
| 8048 | SNIDDRMREVRAVLTETLPFELPLGFTNENLFLS | YP_682736 | Roseobacter |
| | ELRLDQMTGVQQNYLNRLRRPHNNYTKPYLYS | | denitrificans |
| | INRSRRSKNTLGLIHPAVQLRIATFYSEFEQTIIQ | | OCh114 |
| | ACGRSTFSIRHPYEALRIYSKDSAKDVRKRWKL | | |
| | ALPGENVGHAIKTSYTSSYFAYRKYLLLDKFFSS | | |
| | NEIIRLEGKYSRLRMLDVSKCFFNIYTHSISWSL | | |
| | KDKDFSKKNAKNYSFEQQFDTLMQHSNYNETA | | |
| | GILVGPEVSRIFAEIILQRVDVELERAVSKRLKLE | | |
| | CGRDYDIRRYVDDFHLFANDEDVLDKVEGVLA | | |
| | EILETYKLFLNTGKSEEVERPFVTGISRLKFEVSG | | |
| | ICAKLYDELTVDLSNNEESSAEARETLRKARVS | | |
| | LDALRHIGGTERALLPSAMSEVFTTLSRIVRSLN | | |
| | KMTELDLSEAQSEDLIARVKTVVRVLFYLSAID | | |
| | FRVPPIIRLSLILKEVVRLSKKLAQPYRETITGYL | | |
| | VYELSELMATHYVEEAEAVGLEVANTFVLGLV | | |
| | VEPALFAMQESSAKFLHNVLTGKHKCYFSVLC | | |
| | ALHALNSVENIKEDEKNAFVDGLINRILSNDFEI | | |
| | EVSCEEYLIFCDALSCPAIDRDVRWQTFQDKLG | | |
| | GQGLSKAAFDELALCFRHINWDANGPGFELVQ | | |
| | RRLPPVYFSG | | |
| 8049 | PMLKLAERSLDWALLHIEKYGDTDIFPVPFEFEA | YP_519037 | Desulfitobacterium |
| | IRYQWEQSMRSWLRSQDILQWTPRPYRRCLTPK | | hafniense Y51 |
| | HRYGFRVATQLDPLETIVFTSLVYEIGKDIESARI | | |
| | PKEEKIAFSHRFAAKPDGRMYDSEYSWDLFQD | | |
| | HCGELVESNDYRYVVIADIADFYPRIYFHPLENA | | |
| | LSECTRKKNHIKAITSMIKNWNFSVSYGIPVGSA | | |
| | ASRLLAELVIDDVDRGLLSEGVKHCRYVDDYRI | | |
| | FCKNEREAHEHLALLANTLFENHGLTLQQHKT | | |
| | RILPIEEFYNHYLQRENSQELNSLSAKFYHILDSL | | |
| | GIENRYEDIDYDDLAADVQAQIDNLNLMGILKE | | |
| | QVLETEAIDIPLVRFVLKRLKQIDSEESVDFVLD | | |
| | NINQLYPVFKEVITYITSLRSLNTADKHEIGKKLI | | |
| | QLLEDSIVSHLEYHRLWVFDTFTKDREWDNEG | | |
| | KFVNLYNSYHDEFSQRKLILALGRAQQHSWFKS | | |
| | RKRTVNQMSPWLKRAFLAAASCLPGDEAEHW | | |
| | YKSLQGSLDPLELTITKWVQANPF | | |
| 8050 | QSNDEVDYIVDPAFWLNGQALGFPVNFELVLK | ZP_01039369 | Erythrobacter |
| | HLRQDMRDDWYYDCLQYDDLFKDPSEAKRIIIS | | NAP1 |
| | LLQEWNGEYRGTRSVVRNIPKQGYGERYGLET | | |
| | DFFDRFVYQAICSFLIPFYDPLLGHRVLSYRYEP | | |
| | TPIKAKYLFKNKIDRWFTFEGVTLTFRKSGLYLL | | |
| | ITDLSNFFENVSREQIIKALEQAVPNLLATGPQK | | |
| | LHVRNAIATLDRLLGQWTYSGDHGLPQNRDAS | | |
| | AFLSNILLSNVDRKMAEKGYDYYRYVDDIRIIT | | |
| | DSETHARRGLQDLIRELRTVGLNINAKKTEILAP | | |
| | DVSDEKVAKYFPSQNSSTIAINQMWQSRSRRVV | | |
| | TRSVTYIFEILSKCIAEWDTQSRTFRFAVNRVAK | | |
| | LVDSGLFDVGDALSVELLDTLSQSLSEHAVSTD | | |
| | QYCRLIATLDHGGRCLPSLEAFLLAEDGAIHDW | | |
| | QNYNIWMLLAVRQHRSDDLVALAERKLQADM | | |
| | KSGEAAAILIWLRCVDEKALIARCLEEFANLPFQ | | |
| | NARYLLISASVLEEEVLRPLYGHVPTGLRGTGH | | |
| | RTQRHCNEDGLPFAPRENTDLLNLIDEISGYD | | |
| 8051 | LTDRFHQIRKEELENLFSKKNISDVWRKIVRDQL | ZP_01202165 | Flavobacteria |
| | RRVDILDTEDYYDFNYNIDERALLLRTVLLNGN | | bacterium |
| | YQPSQPLIYRIEKKFGICRHLVIPHPLDALVLQVI | | BBFL7 |
| | TENISQQILNNQPSKNSYYSRDKHNLRKPHEIDE | | |
| | YGYHWRRLWKKMQKQIYQFKEEKELIIVTDLS | | |
| | NYYDSIYIPELRKVISGFIDKKESVLDILFKIIERIS | | |
| | WLPDYLPYTGRGLPTTNLEGVRLLAHSFVFEID | | |
| | EVLKSKSNESFTRWMDDIIIGVNSRTEAVNVLSS | | |
| | TSDMLKSRGLALNLKKTNIYSSKEAEFHFQIEEN | | |
| | QYLDSIDFDYHIEHGIRKIGSELSKRFTKHLKNN | | |
| | VSAKYSEKITKRYITSFAKLQSKQLLKKVPVLEN | | |
| | EIPGVRGNLLYYLSSLGFSKRTSEIVLNILKELKL | | |
| | HDDISLFNVCKLVTDWEIPITKESDAFIKAFIKQV | | |
| | KSFSIQRKQPFDFYCLIWVKTKYEHPDELLKFIN | | |
| | DYDYIWKTHPFLRRQVTSIMGRLLNYRKDEITK | | |
| | FLQSQIATSEPQVVSVANSILEFSKIKTVEQKVK | | |
| | MYLFPQSKYRTYPYQKFLVLCSFLNSEVYSKNE | | |
| | DIKKKVLENISDPYYLKWLDYQYNIK | | |
| 8052 | QVTLLREERMRSFLSQVCDQLNMRWAWEKVK | NP_882842 | Bordetella |
| | RASVPGDIWIDEADLAHFEVHLGHELRGLGDDL | | parapertussis |
| | LSGRFRMSPIRPMVFPKNPDGDGNPRVRQYFHF | | 12822 |
| | TVRDQAWVAVVNVLGRYIDEQMPVWSYGNRL | | |
| | FRSAWIEEDIHGNKIRKIGPYRHSSGRIYRLFQQS | | |
| | WPLFRRHIALAVSAAAHGYSKVDSLDDDEREE | | |
| | LGFQRRMHRANQCPFVLADYWTNLPTGPNESD | | |
| | VYWASVDLEKFYPSIPLTACVDAISQFVPAELRP | | |
| | EVQRLLKTLTQLPLNLDGWTDAELKHIELDESR | | |
| | KTFHKIPTGLMVSGFLANAALLPVDQEVQKTLP | | |
| | RGRVAHFRYVDDHVILTKTFDDLITWIDHYKDV | | |
| | IDNLGSGASINPAKTEPKALGELLGTSDTSKRFA | | |
| | GSDLWNRAQKECRLDPEFPTPLMTKTIALVSAI | | |
| | GKTDFTALEDNELSILPQQL | | |
| 8053 | LLPTLRGHATFGVDVRHTQKITGHGMTNKTDK | YP_373506 | Burkholderia lata |
| | YAALAPRIEYLSDVVVLSQAWKKTHTYIRHHN | | |
| | WYADTLELDCSAVNLDGELSQWSAELRDGTYT | | |
| | PKAARLVPAPKSDPWVFGDAEINGWAPVSSSEH | | |
| | FLRPLAHVGIREQTIATAAMLCLADCVESAQGD | | |
| | TSLDALDAQKAGVFSYGNRLFCSWTDQGARAR | | |
| | FSWGNSNVYSRYFQDYQSFVERPLLIAQSAVLS | | |
| | GQDALTLFVIKLDLSAFYDNINIEGLVEKLTELY | | |
| | WRYSETIAPTAKTSSARFWATLAKSLSIGWQVE | | |
| | DAKWAPYLKGQKLPSGLPQGLVSSGFFANAYL | | |
| | VDFDEAVGESIGRSFNRRGVKFRLHDYCRYVD | | |
| | DVRLVVSCDKQVPSEEELGLALTEWVQARLDS | | |
| | KANDVEFEERLVVNEQKTEVQPFASLGGESGTA | | |
| | ARMKSLQSQLSGPFDIAALQHVEAGLNGLLAQ | | |
| | AELGTAAKQKVDGGRNLPPLASVVRPKREVRD | | |
| | DTLTRFAAYRLTKALKLRRKMTDLTQDEEAGL | | |
| | SRNILLHDLEVAARRLVAAWSLNPGLAQVLTY | | |
| | ALDLFPCPELLKTITDALLTKVLGTQEDAYSTGT | | |
| | ALYTLAQLFRAGASQTGKYWESDGSLQVGDVE | | |
| | RYRLELGQLARMLIDEGVLPWYVRQQALLLLA | | |
| | SLSQVITLDTSFDELPYHRSLHEFIGKRVDEGLP | | |
| | HIEETIAVSLVGHQLLRDDAHYAMWFAALSRQ | | |
| | STRKDRLIALELLAQNQPHLLRVIAASRSNRQLA | | |
| | AEPAFQSIVRYFSSFKEPEDECALVDGEWLSFGD | | |
| | IVKMRDTPFHEENALLQLAFALADAVSRTDELP | | |
| | EQWTPQTIQVRCEDWALLSDPRGMPRLSIRIGPT | | |
| | RGRRDPRYRTPEWCNSEDAILYAIGRVIRCAAT | | |
| | GELDFTARQWLLREENIGWYRGISSTWKKRQIG | | |
| | MLNTSVAMAGTTAAITPWFSELLLRLLRWPGL | | |
| | QAQLETSSSVGVVTDAAMLRDLVHERLKAQAA | | |
| | LFGKSSNLPIYVYPVDWPIDESRLLRVCVIQGLL | | |
| | PTTKDFDGGLASLHKEGFRARQRNHTASILYLA | | |
| | YQQLQARDSVLGKDHKPYVDLVVLPEYSIHLD | | |
| | DQDLMRAFSDATGAMIFYGLCGATHPVTSEPIN | | |
| | AARWLVPQRRNGGRSWVEVDQGKKYPTTEEE | | |
| | TLGVKPWRPHQVVIELSSGGAGKFRISGAICYD | | |
| | ATDISLPADLRDVSHMFIVSAMNKDVKTFDSM | | |
| | VGMLRYHMYQHILVANAGEFGGSTAQAPYEQE | | |
| | HKRLISHNHGSDQISVSVFDVDINHFGPNLQATK | | |
| | VTLDPSGKKKRIGKTPPAGLMRDSVQ | | |
| 8054 | RPLAHVSIKDQTIFTALMMLLANHVETEQGDTS | AAR05370 | Aeromonas |
| | TSFYDVHAKGLINYGNRLHCKYSDNNAIYSWG | | hydrophila |
| | NSNTYSKFFTDYQRFLERPIHFGREAKRVKTSKE | | |
| | EIYEIHLDFSKFYDSVNRGILTKKISALVEKITGA | | |
| | ETDECISHVLSKFRNWKWTEKSKELYTGVCKN | | |
| | KHIETLKDNRGIPQGLVAGGFLANIYMLDFDKA | | |
| | ISKLIGQYLDDNETILLIDACRYVDDLRLIIKADK | | |
| | NEVSENKIREVITTRFKSYYDELELILQPQKTKV | | |
| | KKFSSKDGAISSKLAHIQNKISGPMPLHELDEQL | | |
| | GHLEGADRTNRQP | | |
| 8055 | KRVGLLFERVVAFENLLHATRQAARGKKSQLR | ZP_00592520 | Prosthecochloris |
| | VAHFLFHQEKECLRLQTELKQGIWQPSGFRVFEI | | aestuarii |
| | REPKPRRISAADFQDRVVQHALCNILGPLCERRL | | DSM271 |
| | IFDTWACRRGKGSHLAMKRAQAFSRRFPYFLK | | |
| | CDIRRYFDSVDHTILKRLLWRLIKDKPVLNLLD | | |
| | RIIDHPLPGALPGKGLPIGNLTSQHFANLYLGEL | | |
| | DHQLKDRMGVKAYLRYMDDMLIFADDKSRLH | | |
| | ELVTGIEDFVKQHLQLSLRPSATLVAPVSEGVPF | | |
| | LGFRIFPGLVRVNGQALRRFRHRLRLHEKAYQT | | |
| | GKMDVESLTASVQSMIAHLQHADTHRLRQSLL | | |
| | SSSCALG | | |
| 8056 | FFLRIVMKRIGNLYESVVSGESLWEGYLGAKKS | AAZ18310 | Psychrobacter |
| | KGGRRGCFQFEKSLGRELNELQEELANNTYKPR | | arcticus 273-4 |
| | PYFKFIVYEPKKREIYAPAFRDCVVQYAIYLRV | | Bacteroides |
| | MPIFDKTFIDQSFACRTGLGTHKAAEYAQDALR | | |
| | RAGPNTYTLQLDIKKFFYSIDRPTLRKLLERKIK | | |
| | DKRLVDLMMLFADYPEPKGIPIGNLLSQMFALI | | |
| | YMNPVDHYATRVLKPAAGYCRYVDDFLLFGLT | | |
| | RAQALTYRKLLTDFVEQKLKLTLSRSTIANTKR | | |
| | GANFCGYRTWRSGRFIRKHSLYKTRKAVRANK | | |
| | LESVISHLAHASKTHSLQHLLNYAEQQNHGLYC | | |
| | QLPKIYHTRHHQAVERSGRINGVMRNRCSNVC | | |
| | IDNKLFTFQQIYEAYRKLKSYIYYDNTSLFIRKEF | | |
| | SSFESSILAGENFEDNFKKKMKSLYDILNSDNYQ | | |
| | TDLDKLVSKIGYKLVPKSIKKKESTIVTNIPHTG | | |
| | KIEVESYNILIDAPIEIHIISVLWLIVAGKELCKYV | | |
| | NENNYAYKLLLLDESYLPMTDIKIETNKENYVV | | |
| | TGLQLYEPYFIGYQNWRDNALNSATKLLDDNK | | |
| | DATILSLDIQRYFYSVRIDLDSIKNRCSHSNKQIE | | |
| | KCFHLLQIINKTYTSKINKLLDIPLTNDELNAGY | | |
| | TILPIGLLSSGLLGNLYLEDFDKTIKEELNPAYYG | | |
| | RYVDDILFVFSDRKVKLEVNNPIHDFIDRYFIKK | | |
| 8057 | NILETILDENNITYLLPVGSHKLKIQSEKVILEHF | YP_212908 | fragilis |
| | NHKESRAAINIFKKNLDKNRSEFRFLPYEENIDN | | NCTC9343 |
| | EFDNEAFSMHYSDSINKLRSIKEFKEDKYGASKF | | |
| | LAHKIFLSKISPNEKDYNRKYFFQSSKQILTFFKG | | |
| | STALSLYTLWEKVATYFVINNESKCLIIFYNQVL | | |
| | NTINNIKTVNGENKIKEDLKEFLFISIAMPISMRM | | |
| | NIKIDNNNDDVVIHKLADDIRKTNMFRQNLLGI | | |
| | SCINYTDYIDHITNDLFHINIKNIKDLKIQINIAKFI | | |
| | SPRFVHYHEFNILNIYQVFSSINKESDIRLFDKIQ | | |
| | DIAFNQYKEFNYDWRFLYSNTDPIYPKDFFTLT | | |
| | QSENSNCKNINILEDNEVECNKKIGLANIEVSEN | | |
| | DILSAIKMIPNISKERRKRIFEIMNYSHKKHVDLV | | |
| | VLPEVSVPFEWIDFFAEQSKNNNIAFIIGLEHVVS | | |
| | SYNFAYNFTATILPIKQKEFTTCLVRIRLKNYYS | | |
| | HSEKELLKGYRLINPSEIKPELKLYDLFHWRKSY | | |
| | FSVYNCFELANISDRALFKSKLDFIVATEYNKDI | | |
| | NYFSDIAGSWVRDLHCFFVQANSSQYGDSRIVQ | | |
| | PAKSDFKNMISVKGGKHPVVLIDELQIDKLRDF | | |
| | QNKEYNLQKELIDKEKTHLKPTPPDFNKDNVLK | | |
| | RIKDEPI | | |
| 8058 | REQSFDKFSLSKRIKQSDFYKCKNLVDEKVFDQ | YP_049163 | Pectobacterium |
| | VIEESYQLAHGLTAPVISKTISKGKEVYYVDRLS | | atrosepticum |
| | YKLILRKLQGNIRNKIETDKLQRNEIVRNLVSYL | | SCRI1043 |
| | QEGVKSKVICIDLKSFYESIDIDSLLSETAKIGSLS | | |
| | YHSKKLIEVVLDEHRSIGGKGVPRGLELSSLLAD | | |
| | LYLQEFDEWIKRIDGVFLYKRFVDDILIMTDHK | | |
| | VDEQSILTSIKNKLPANLCINNLKTQIIEIKKRTH | | |
| | SPNDVEGKLAATIDYLGYKIKVIDTHIPPAANGA | | |
| | SSEGKANSVYRKVVIGVSDKKLNKIKTKLCKAF | | |
| | YNYELNRDFTLLLDRIIFLSTNRLLINKDKNRKM | | |
| | PTGIFYNYPLVNDDNESLKVIDFYIRALILGSGC | | |
| | RLSKKLNGSLNNGQVKTLLKISFAKGFSNKIHK | | |
| | KYSLNRLKEITRIWK | | |
| 8059 | VGRHEKNSVLEISFKYAPRGVELVKARSEGAAP | CAE73057 | Caenorhabditis |
| | LPTEPRGQRESARPIAYIKARTTAVRRVTLSVVH | | briggsae |
| | RYGRMHTLATKKLRKISCPPTFKKETKFLKSSEI | | |
| | EKFKNSQKKVFDVEALYTNIDNRAAYQAVVDK | | |
| | LKKNASVIDWYGDSFTHIKMLLKSCLEFNGFQF | | |
| | DGRIYEQKRGLAMGSRLAPVLAVLYMDIIETPS | | |
| | KVHPIILFRSYIDDYIVVAESQDTLDNIFTCLNSQ | | |
| | ATHIRLTREAPKMDWLSFLNCELRFKNKVFSSR | | |
| | WYRKPSNKNLLIRMDSGHPKQQKINTISTTQKT | | |
| | ATENSTIDQRSYSRNLANEGNFDGKKNRLSNAK | | |
| | KSENFRSSLQNRIDFV | | |
| 8060 | DVEIQFENLYAQTAELVSSSKDNVESFKSTLVD | CAJ00247 | Schistosoma |
| | CCFRYLNHGHSSKGILTNKHKEALRKLKTNDNL | | mansoni |
| | LITKPDKGYGIVLMDKNNYINKMKAFLNDQSK | | |
| | FQKLVVKNDLADKIEKQIIDSLKQIKQQGFISEK | | |
| | VFEMLKSIGTRTPRLYGLPKIHKSGLPLRPVLDM | | |
| | NNSAYHTIAKWLMQILKPLHKEIVKHSVKDSFE | | |
| | FVNNIKNLSLKNKFMISLDVTSLFTNIPLLETVDF | | |
| | ICNELTERHTETVIPVTAIKQLILRCTMNVQFRF | | |
| | DNEYYRQLDGVAMGSPLGPILADIFLAKLENGP | | |
| | LKDTISHLTSYCRYIDDTFIVLEKEHEKENILNIF | | |
| | NNIHPSITFTLEEEQNGSISFLDVQLTRRIDGTLK | | |
| | RGLHRKSTVGQYTHFYRAVSIK | | |
| 8061 | RSELIEIMNQCRAFRLTMDLKRYYLTKPNGKYR | AAD12231 | Fusarium |
| | PIGSPTLGSKVISKALTDIWTTIADKRRGVMQHA | | oxysporum |
| | FRPKLGVWSAAFAVCQKLRSRKPSDVIIEFDLK | | |
| | GFFNTIKRNSVQEAANRFSLLLGNCVRHIIDNTR | | |
| | YVFEELKPETELHIINDYTHHKYKRAIPIYRTGV | | |
| | PQGLPLSPVAATIALENEVNMPEMVMYADDGIL | | |
| | IGGKEKFAEFVKKAIRVGAEVAPEKTREVTKEF | | |
| | KFLGLTFNLEKETVSNGDSYRFWNDKDL | | |
| 8062 | DNIHTAKHLISPHCYLASIDLQDAYYSIPVDPNS | XP_0011926 | Strongylocentrotus |
| | RKYLRFMWQGERWQFAALTNGLSTAPRLFTKL | 93 | purpuratus |
| | LKPVFAELRQAGHTVIGYLDDTINIGETKEKLKE | | |
| | SVMRQHFENRGLSRRTVDIITASWRASTCKQYQ | | |
| | VYITQWRRFCHSRNTSYLQAEVETVLEFLSSLF | | |
| | HDRNLSYRNDNYPSRLQEARVPLLPRSTCTRQN | | |
| | VYGNKLTPQMLCAGYLRGGIDSCDGDSGGPLV | | |
| | CENSNSVWKVVGVTSWGYGCAQPNAPGVYAV | | |
| | VT | | |
| 8063 | SPSRWLIRTIRLGYAIQFAKRPPKFTGVYFSRVN | XP_689703 | Danio rerio |
| | PLSAPVLREEIAALLAKGAIEPVPPAEMESGFYS | | |
| | PYFIVPKKSGGSRPILDLRVLNRCLHKLPFRMLT | | |
| | QRRILQCVRPRDWFAAIDLKDAYFHVSILPRHR | | |
| | QFLRFAFEGRAWQYKVLPFGLSLSPRVFTKLAE | | |
| | GALAPLRLAGIRILSYLDDWLILAHSREQLIMHR | | |
| | DEVLRHLRLLGLQVNREKSKLAPVQRISFLGME | | |
| | LDSITMRLLGHMASAAAVTPLGLLHMRPLQHW | | |
| | LHDRHRVSVTALCRRALSPWNDPSFLQAGVPL | | |
| | GQASSHVVVSTDASNTGWGAVCRGHAAAGLW | | |
| | KGAQLHWHINRLELLAVFLALHRFLPVLERQH | | |
| | VLVRTDSTAAAAYINRMGGMRSRRMSQLARRL | | |
| | LLWSHPRLKSLRAIHVPGTLNRAADALSRQLLR | | |
| | PGEWRLHPESVQLIWARFGEAQIDLFASPENAH | | |
| | CQLFFSLTEGSLGTDALAHSWPRGMRKYAFPPV | | |
| | SLLTQFLCKVREDEEQVLLVAPLWPNRTWISEL | | |
| | SLLATALPWRIPLREDLLSQGQGTIWHPRPDLW | | |
| | NLHLPLKKQTVTGSIIAESLKGYML | | |
| 8064 | LPPTGALSQTLQSLTDSKIQEVEKLRGLYESQKT | XP_001217723 | Aspergillus |
| | SILHEADQVTNHQERVARILVGIKRYYPNEYHD | | terreus NIH2624 |
| | PEVRNIEQLLDQARYDSSIPPETLQKFESQLRAR | | |
| | LETKSRRLGLADLYCRLLTEWMQPPTDPEKGK | | |
| | EVATIEDDFLLVEGKQKQKLKELCDQFESVVFE | | |
| | PRETNADEIRGFLDGLFATEESAKALEELRARIH | | |
| | LQCITFWEEEEPFNPDALAMCIRGLLTEDLLSEE | | |
| | KQDTLKYILENKVALREISDVLNMRYADLGNW | | |
| | DWRAGKDGIPVLPRQQVNGKYRIWMDEDVLQ | | |
| | AVFTQYIFIRLCNMVKETLTDFIGDGRVWDWG | | |
| | RSREMTERDKLRWKYYFNLSSPASFGVDAVRK | | |
| | QEFLERHFLYQIPSTQTTLQERGGAYDDDDGDE | | |
| | GSYAAPSEVPKNIKQQLLRKIATETLIQRQVNGR | | |
| | AAVVQSDLQWYATALPHSTIFAVVKYLGFPEK | | |
| | WIRFFEKYLKTPLNLIRSFEDSQSGPRIRHRGVP | | |
| | MSHASEKFLGELVLFFMDVTVNRKTGMLLYRM | | |
| | HDDLWFCGEPEQCVKTWEVLQKYARITGLDEN | | |
| | YSKTGSVYLADIVDEAVVSKLPQGPVKFGFLIL | | |
| | DPKSGSWVIDHSQVEAHIQQLKKQLDHCDSIIS | | |
| | WIRTWNSCIGRFFRSTFGEPAFCFGRPHVDAILA | | |
| | TYAKLQNTLFDDQRQCGALRVTEFLRQRIKSHY | | |
| | GEFDIPDSFFFLPEELGGLGLRNPFVPIMLVRGNI | | |
| | ENSPIDLCDKFKKEEIEDYVAAKKAFEDLHERA | | |
| | RLRRLDYINREADSRLGQIIKTSEMNHFMSFEEY | | |
| | TRFRESKSTRLRSCYEELMRVPERMSLFSTQIVR | | |
| | DELHRTGRSRLGHLDLETKWLLNLYADELLAN | | |
| | FGGFDLVDKKFLPVGVLNMVKGKKVKWQMVL | | |
| 8065 | AASSLSTLQHVTLQKLNKLDSQRQQFESDKKSI | EAT92517 | Parastagono |
| | LEQVSSVPDHRSKVEALLDGFELHGIAPKQADL | | sporanodorum |
| | SISNLKHFVHQAKHDPSVSASLLKDWQSRLEHE | | SN15 |
| | LNVKSNKYEYAALFGKLVTEWIKHSTLVKSAD | | |
| | VSDGSIAKGRKKMQEQRQSWENYAFVEKEVN | | |
| | QSTIEQYLSDIFGDALQTEKIKKSPLRVLRDSMK | | |
| | EVMDFKSDLDTSEKDFSSNKRFGHSAPHGSRFTI | | |
| | ELLQSCIRGVKKADLFTGRKLEMIIDLEKQPAVL | | |
| | KELVDVLNMDVDGLDHWEWDGPVPLNMRRQ | | |
| | TNGKYRVYMDEEIHQAILLHFIGKTWAVALKK | | |
| | AFTNFYHSGAWLQAPYRSMPKKIRQRREHFIEN | | |
| | SNKSGDSVRNYRRQKYQQEYFMTQLPSNAFED | | |
| | AREYDAAEGQEKNSHIATKQTMLRLLTTEILLN | | |
| | TKVYGECSVLQSDFKWFGPSLPHDTIFAVLEFF | | |
| | GVPAKWLRFFKRFLEVPVVFAQDGAGAKARVR | | |
| | KCGIPNSHILSDALGEAVLFCLDFAVNRRTKGA | | |
| | NIHRFHDDLWFWGQETTSVQAWEAIKEFTEVM | | |
| | GLQLNEEKTGSSIIVADKSRARVPHPNLPEGNLH | | |
| | WGFLELDASAGRWVIDRAQVDEHITELRRQLD | | |
| | ACHSVMAWIQAWNSYVGLFFNTNFAQPANCFG | | |
| | RQHNDMIIETFSHIQRSFFGKYGTANVTEYLRSV | | |
| | LKERFQTTDAVPDAFFYFPVELGGLGLNNPSISA | | |
| | FATYQNSSRDPSARIERAFEEEREAYDTAKQRW | | |
| | DAGDVPCPNRETDEPFMSFEEYTAFREETSHPLF | | |
| | EAYMNLLECPVEERVETSDEMYEALRRSDAPH | | |
| | ALGSNHYWLWIFNLYAGDLKQRFGGQGVQLG | | |
| | ERDLLPVGLVEVLKSEKVRWEN | | |
| 8066 | ALVDTGAETSIIYGDPNQFSGSKAMIGGFGGQM | XP_001234064 | Gallus gallus |
| | IPVTQTWLKLGVGRLPPREYKVSIAPIPEYILGID | | |
| | ILSGLTLQTTVGEFRLRERCISIRAVQAIIRGHAEI | | |
| | EPICLPQPRRITNTKQYRLPGGQQEITKTVQELE | | |
| | RVGIIRPAHSPYNSPIWPVRKPDGTWRMTVDYR | | |
| | ELNKVTPPIHAAVPNIASLMDTLSREIETYHCVL | | |
| | DLANAFFSIPIAKESQDQFAFTWEGRQWTFQVL | | |
| | PQGYVHSPTFCHNLVASDLANWNKPSTVKMFH | | |
| | YIDDLMLTSDSIEALEKTVPSLITYLQEKGWAIN | | |
| | PQKVQGPGLSVKFLGVVWSGKTKVLPSAIIDKI | | |
| | QAFPVPTKPKQLQEFLGILGYWRSFIPHLAQLLK | | |
| | PLYRLTKKGQVWDWGRTEQEAFQQAKIAVKQ | | |
| | AQALGIFDPTLPAELDVHVTQEGFGWGLWQRQ | | |
| | GSVRIPIGFWSQIWHGAEERYSMVEKQLLATYS | | |
| | ALQAVEPITQTAEVIVKTTLPIQGWVKDLTHIPK | | |
| | TGVAQSQTVARWVAYLSQRSRLSSSPLKEELQK | | |
| | ILGPVTYHNGSSRGNPSRWRAVAYHPSTETIWF | | |
| | EEGDGQSSQWAELRAVWMVITQEPGNSALNIC | | |
| | TDSWAVYRGLTLWIAQWATQDWTIHARPIWG | | |
| | KDMWVDIWNVVRHRTVRAYHVSGHQPLQSPG | | |
| | NDEADTLARVRWLGNTPSEDIAHWLHRKLRHA | | |
| | GQKTMWAAAKAWGLPIQLPDIVQACQDCDAC | | |
| | SRMRPRPLPETTAHLARGHNPLQRWQIDYIGPL | | |
| | PRSEGARYALTCVDTASGLMQAYPVAKANQA | | |
| | NTIKALTRLMASYGTPEVIESDQGTHFTGATVQ | | |
| | KWAEDNNIEWRFHLPYNPTGAGLIERYNGILKA | | |
| | ALKADSQSLQGWTKRLYETLRDLNERPRDGRP | | |
| | SALKMLQTTWASPLRIQITSKDTSLKPQVGTMN | | |
| | NLLLPAPDDLEPGRHKVKWPWKVQAGPKWCG | | |
| | LLAPWGRLLEVGGSVNPSVIGVWPTEVIVDTPV | | |
| | FIARGTLIMSMWQIRTPPLVPDIVIQSQISGQRV | | |
| | WYRRPGRAPIQAEVLTQDRNTACILPWRADLPL | | |
| | LVPIKHLFYSP | | |
| 8067 | VKSLCQPRKHQGGHQVIHEFLCIPECPLPLPGRD | XP_874426 | Bos taurus |
| | LLSKLGVQVTFSPEERPTFRVGTPTNLLSLSVTP | | |
| | QDKWRLREPPGDKQGQATEVERRLTQLFPEVW | | |
| | GEDNPPGLARHQAPMIIELKACATLVRKCQYPIP | | |
| | RGARIGILPHISRLKQAGILVECQTAWNTPILPV | | |
| | KKEGGQDYRPVQDLRLVSQATVTLHLSVPKPY | | |
| | TLLSLLPPKTRIYTCLDLTEAFSRICLAPASQPIFA | | |
| | FEWDDPIGGNKQQLTWTHLSQGFKNTPNIFGEA | | |
| | LASDLEPFQPERYGCCLLQYVDDGLLAAETWV | | |
| | ECCEGTPVLLHLRAEAGSRVSRKKAQICKEEIR | | |
| | YLGFVLRGGTRLLDQSRKEVILRPHTPKTHRQV | | |
| | REVLGATGFCRIWIPRYSQIAQPLFELLTGPEENP | | |
| | INWTEKQQKAFEELRLAFTSACALVLPDLPKPF | | |
| | TLYVTGKDEQPWGF | | |
| 8068 | PEIANDGSQSHITFFLCASLLFPMEARSYWGDSP | XP_693232 | Danio rerio |
| | CCSPQLDDIVDHDGLQTKRSSSSPQGAPKKRTA | | |
| | FVDITNAHKIELCNPIKKKDPAKKVQKTSVLLK | | |
| | NDVNLKSIVSPEEKLEELKNVESDIEETSCKDPIP | | |
| | PHLLPPEIPPEFDIDSEHLSDSSHTSEYAKEIFDYL | | |
| | KNREEKFVLCDYMVDQPNLNTNMRAILVDWL | | |
| | VEVQENFELNHETLYLAVKVTDHYLAVSQTKR | | |
| | EALQLIGSTAMLIASKFERVEDICLNWRNGHLT | | |
| | NILLCAQHAEDKLTVKQEQDKKKAERELCMAL | | |
| | VQVANQRSNQHLTTKFKGDKGVKGEKDTRWR | | |
| | NWYVTYGDETCRACRARDVNRYQMSALATEM | | |
| | DFWLRIDPKGANQQRGADVRRGKSRMERVWA | | |
| | NSQQLQDIMSPGELHCTSHVVTDRDAEFEKAW | | |
| | FEANVNETLILEKMYWKDSLCAVSVSLSEKQRS | | |
| | FYLMSAQAVPHISVCKGKHQSWADLGPFEKQC | | |
| | LEVKDCISREGGVEWSALSQAFRVDSETETAVS | | |
| | RTVTAIDKHCVKNSCMVDFNAADIHPALAEILS | | |
| | ELWAKSKYDVGFIKGCDPVTIIAKSDYRPCQQQ | | |
| | YPLKREAIEGITPVFEALLEQGVIVPYNNSKVRT | | |
| | PIFPVKKIRDNGMPTEWRFVQDLQAVNAAVKQ | | |
| | RAPLVPNPYTILSQIPEKSQFYSVVDLANAFFSV | | |
| | PVDKDSQFWFAFNFNGKGYTFTRLCQGFTASPT | | |
| | LYNEALLRSLEPLTLTAGTALLQYVDDLLICAE | | |
| | NEETCVKDTVTVLRHLAKEGHKDMQTFATGLE | | |
| | KNKWRQSGCVMKDNVKAAQGKAAERPLHSLR | | |
| | PGDFVVIRDLRRKSWRAKLWLGPFQVLLTTETA | | |
| | VKVAERATWVHAGHCWKVPSPEKDSTRE | | |
| 8069 | VHGTLLVLQGPFVSAGGHLVIHEFLYLLGSPIPL | XP_607546 | Bos taurus |
| | PGRDLLTKLGAQITFAPGKSASLTLGRQSALMM | | |
| | AMTISREDEWCLYSSGREQINPPRLLKEFPDVW | | |
| | EEKWPPGLAKNSVPIVVDLRPGATPVRQKQYPV | | |
| | SQEACLGIWDHIQHPQNAEILIECPSPWNTPILLV | | |
| | KRSGGNDYRAIQDLRTINCSDHHPSSGPKFLHSL | | |
| | ESLTHSGKLVHLPRSHGLILLPPAVTSQPLFAFE | | |
| | WEDPHTGRKTQLTWTQLPQGFKNSPILFGEALA | | |
| | ANLAAFPSETFNCTLLLYVDNLLLASSTQGDCW | | |
| | RGTKALLALLSTTGYKVSWKKAQICRQEVKYL | | |
| | GFVITKRHWVLRHERKRAICSIPWRDTKKEV | | |
| 8070 | VVGTLPLNLLGLDMLKGKSWTDDKGREWMFG | NP_989963 | Gallus gallus |
| | VPSLNIRLLQTAPPLPPSNLTCVKPYPLPLGARS | | |
| | GISPVLAELKEQGIVIPTHSPFNSPVWPVRKPNG | | |
| | KWRLTIDYRRLNANTGPLTAAVPNISELIAAIQE | | |
| | QAHPFMATIDVKDMFFMVPLHPDDQLRFAFTW | | |
| | EGQQYTFTRLPQGFKHSPTLAHYALAKELEQIP | | |
| | LEEGVRLYQYIDDILIGGDHLTPVKIMHDKIIKR | | |
| | LEELGLTIPPDKIQSPAAEVKFLGIWWKGGMACI | | |
| | PQDTLSALDQLKMPENKKELQHALGLLVFWRK | | |
| | HIPDFSIIARPLYDLLRKGVSWGWTPVHEEALQL | | |
| | LIFEAITHQSLGPIHPSDPVQIEWGFAHSGLSIHL | | |
| | WQKGPEGPIRPIGFYSRSFKDAEKRYSQLEKGLF | | |
| | VVSLALREAERTIRQQPIILRGPFKVIKSVMSGTS | | |
| 8071 | PPDGVAQRASVRKWYAQIEHYCNIFKVTEGAP | AAA73090 | Homo sapiens |
| | KTLAIQDDILSTTDTDLPSVVQVAPPYSDQLQN | | |
| | VWFTDASSKREGKVWKYRAVALQIGTDLTIITE | | |
| | GEGSAQVGELVAVWSVFQHESESTTRVHIYTDS | | |
| | YAVFKGCTEWLPFWEKNNWEVNRIPVWQKEK | | |
| | WQDIISIAKKGQFSVAWVAAHQEDGTPVSHWN | | |
| | NRADELARIAPLRQGEPDSDNWERLVEWLHVK | | |
| | RGHTGALDLYRETQARGWPVTREQCRTCISAC | | |
| | DLCRTRLGQHPLQDAPLHLREGKHLWETWQID | | |
| | YIGPFRKSEGKQYVLVGVEIISGLLQAESCPRAT | | |
| | GENTVKALKKWFSILPKPTSIQSDNGSHFTSGVV | | |
| | QEWAREEGIHWIFHTPYYPQANGIVERSNGLLK | | |
| | KFLKPEKTNWSTRTSDAVRRVNDRWGINGCPR | | |
| | FNAFYPKAPPLLPITLNPDKLEEPSYSPGQPVLV | | |
| | DLPHVGPVPLTLMESLNKYTWRAKDAREKEYK | | |
| | INARWIIPSF | | |
| 8072 | TVGGKDIDFLVDTSAEHSVVTASVAPLSKKTIDI | O14746 | Homo sapiens |
| | IGAMGVSAKQAFCLPQTCTIGGHKVIHQFLYMP | | |
| | DCPLPLLGRDLLSKLRATISFTEHGSLLLKLPGT | | |
| | GVIMTLMLPREEEWRLFLTEPGQEIRPALAKRW | | |
| | PRVWAEDNPPGLAVNQAPVLIEVKPGVQPVRQ | | |
| | KQYPVLREALEGIQVHLKCLRTFRIIVPCQSPWN | | |
| | TPLLPVPKPGTKDYRPVQDLRLVNQATVTLHPT | | |
| | VPNLYTLLGLLPAEDSWFTCLDLKDAFFSIRLAP | | |
| | ERQKLFAFQWEDPESGVTTQYTWTQLPQRFKN | | |
| | SPTIFGEALARDLQKFPTRDLGCVLLQYVDDLL | | |
| | LGHPTAVGCAKGTDALLRHLEDCGYKVSKKKS | | |
| | SDLPTAGMLLGIYYPTGGAQPRIRKKAGHL | | |
| | PRAPRCRAVRSLLRSHYREVLPLATFVRRLGPQ | | |
| | GWRLVQRGDPAAFRALVAQCLVCVPWDARPP | | |
| | PAAPSFRQVSCLKELVARVLQRLCERGAKNVL | | |
| | AFGFALLDGARGGPPEAFTTSVRSYLPNTVTDA | | |
| | LRGSGAWGLLLRRVGDDVLVHLLARCALFVLV | | |
| | APSCAYQVCGPPLYQLGAATQARPPPHASGPRR | | |
| | RLGCERAWNHSVREAGVPLGLPAPGARRRGGS | | |
| | ASRSLPLPKRPRRGAAPEPERTPVGQGSWAHPG | | |
| | RTRGPSDRGFCVVSPARPAEEATSLEGALSGTR | | |
| | HSHPSVGRQHHAGPPSTSRPPRPWDTPCPPVYA | | |
| | ETKHFLYSSGDKEQLRPSFLLSSLRPSLTGARRL | | |
| | VETIFLGSRPWMPGTPRRLPRLPQRYWQMRPLF | | |
| | LELLGNHAQCPYGVLLKTHCPLRAAVTPAAGV | | |
| | CAREKPQGSVAAPEEEDTDPRRLVQLLRQHSSP | | |
| | WQVYGFVRACLRRLVPPGLWGSRHNERRFLRN | | |
| | TKKFISLGKHAKLSLQELTWKMSVRDCAWLRR | | |
| | SPGVGCVPAAEHRLREEILAKFLHWLMSVYVV | | |
| | ELLRSFFYVTETTFQKNRLFFYRKSVWSKLQSIG | | |
| | IRQHLKRVQLRELSEAEVRQHREARPALLTSRL | | |
| | RFIPKPDGLRPIVNMDYVVGARTFRREKRAERL | | |
| | TSRVKALFSVLNYERARRPGLLGASVLGLDDIH | | |
| | RAWRTFVLRVRAQDPPPELYFVKVDVTGAYDT | | |
| | IPQDRLTEVIASIIKPQNTYCVRRYAVVQKAAHG | | |
| | HVRKAFKSHVSTLTDLQPYMRQFVAHLQETSPL | | |
| | RDAVVIEQSSSLNEASSGLFDVFLRFMCHHAVRI | | |
| | RGKSYVQCQGIPQGSILSTLLCSLCYGDMENKL | | |
| | FAGIRRDGLLLRLVDDFLLVTPHLTHAKTFLRTL | | |
| | VRGVPEYGCVVNLRKTVVNFPVEDEALGGTAF | | |
| | VQMPAHGLFPWCGLLLDTRTLEVQSDYSSYAR | | |
| | TSIRASLTFNRGFKAGRNMRRKLFGVLRLKCHS | | |
| | LFLDLQVNSLQTVCTNIYKILLLQAYRFHACVL | | |
| | QLPFHQQVWKNPTFFLRVISDTASLCYSILKAK | | |
| | NAGMSLGAKGAAGPLPSEAVQWLCHQAFLLKL | | |
| | TRHRVTYVPLLGSLRTAQTQLSRKLPGTTLTAL | | |
| | EAAANPALPSDFKTILD | | |
| 8073 | QKINNINNNKQMLTRKEDLLTVLKQISALKYVS | O77448 | Tetrahymena |
| | NLYEFLLATEKIVQTSELDTQFQEFLTTTIIASEQ | | thermophila |
| | NLVENYKQKYNQPNFSQLTIKQVIDDSIILLGNK | | |
| | QNYVQQIGTTTIGFYVEYENINLSRQTLYSSNFR | | |
| | NLLNIFGEEDFKYFLIDFLVFTKVEQNGYLQVA | | |
| | GVCLNQYFSVQVKQKKWYKNNENMNGKATSN | | |
| | NNQNNANLSNEKKQENQYIYPEIQRSQIFYCNH | | |
| | MGREPGVFKSSFFNYSEIKKGFQFKVIQEKLQG | | |
| | RQFINSDKIKPDHPQTIIKKTLLKEYQSKNFSCQE | | |
| | ERDLFLEFTEKIVQNFHNINFNYLLKKFCKLPEN | | |
| | YQSLKSQVKQIVQSENKANQQSCENLFNSLYDT | | |
| | EISYKQITNFLRQIIQNCVPNQLLGKKNFKVFLE | | |
| | KLYEFVQMKRFENQKVLDYICFMDVFDVEWFV | | |
| | DLKNQKFTQKRKYISDKRKILGDLIVFIINKIVIP | | |
| | VLRYNFYITEKHKEGSQIFYYRKPIWKLVSKLTI | | |
| | VKLEEENLEKVEEKLIPEDSFQKYPQGKLRIIPK | | |
| | KGSFRPIMTFLRKDKQKNIKLNLNQILMDSQLV | | |
| | FRNLKDMLGQKIGYSVFDNKQISEKFAQFIEKW | | |
| | KNKGRPQLYYVTLDIKKCYDSIDQMKLLNFFN | | |
| | QSDLIQDTYFINKYLLFQRNKRPLLQIQQTNNLN | | |
| | SAMEIEEEKINKKPFKMDNINFPYYFNLKERQIA | | |
| | YSLYDDDDQILQKGFKEIQSDDRPFIVINQDKPR | | |
| | CITKDIIHNHLKHISQYNVISFNKVKFRQKRGIPQ | | |
| | GLNISGVLCSFYFGKLEEEYTQFLKNAEQVNGSI | | |
| | NLLMRLTDDYLFISDSQQNALNLIVQLQNCANN | | |
| | NGFMFNDQKITTNFQFPQEDYNLEHFKISVQNE | | |
| | CQWIGKSIDMNTLEIKSIQKQTQQEINQTINVAIS | | |
| | IKNLKSQLKNKLRSLFLNQLIDYFNPNINSFEGL | | |
| | CRQLYHHSKATVMKFYPFMTKLFQIDLKKSKQ | | |
| | YSVQYGKENTNENFLKDILYYTVEDVCKILCYL | | |
| | QFEDEINSNIKEIFKNLYSWIMWDIIVSYLKKKK | | |
| | QFKGYLNKLLQKIRKSRFFYLKEGCKSLQLILSQ | | |
| | QKYQLNKKELEAIEFIDLNNLIQDIKTLIPKISAK | | |
| | SNQQNTN | | |
| 8074 | SASFPSIPGFAGPLSLKAFLEEYFGLHLTFAAETA | AAO67516 | Leishmania |
| | SPSPRAAATAETPSAAGFRALRDVVLPPNQSFL | | amazonensis |
| | VVVYVALHASSSPPPTTAHASPTPATPDPGCAA | | |
| | PPTGLGRLRQPLAHQTAVSTAHDTCVTRCNPSD | | |
| | RQNPFCLSSSSTGKNGNARSPWCASASWLLYTN | | |
| | TSHRPFTDALLRHPWRASFSACLGPAAMGFIEM | | |
| | YCPIVLQLEAMAGGVQVIGPALKHVALETSFSA | | |
| | TAQLSEMKGLPPRGSASVSSRGVKRAVDTGECS | | |
| | APLPLQKQRRVEAVASPPKAGRRLQREVAPSHR | | |
| | PCRDDSFSSPVNRAAMPATWAAALTRTDDPRT | | |
| | RLYSVRVSDSDCTGDGGGALPLPAGSLEAHWL | | |
| | PRHPRSLHRVLQAALPKRAAYGASTRHYCAGT | | |
| | GERGTGCTSVKNISMWHVAHVFRWLVLQPSQS | | |
| | GEVAFQTTSPKFDLPSYLRRFLSTDVDQCSRLDL | | |
| | RGAALRHTGYLEEAFRRQQQGVEPWDVQRLST | | |
| | PVDVVVSYLRTLLPTLRWAPLKEANNGLFWGR | | |
| | DAAGSERVLDALMRAVRGWLIAGRQAVVPVS | | |
| | RFLDGVPVAQVPWLNGFYTTTPSLPFSDATSSA | | |
| | SRHARRERSQVQQRVWLQFVLFLTQDILPFLLR | | |
| | ASFTITWSSKNTHKLLFFPAFVWRRLVRREVRR | | |
| | TRSCHAPRSQMSLAEERTRMSGADHAALVSAP | | |
| | NACGGAVAAAFPSPHSAASNASAAISAPRDEW | | |
| | RAVRTGGALAHWSARATLATRGGGASCLYAG | | |
| | VRFRPDRRKLRPIAVVRSASLRSLKEMARGSPSP | | |
| | YSHASAIVRLLRRLGCSDADGQLPAATATLLRQ | | |
| | VQARSRHNRRTGVHRSAHPLPPHLPHKAALQD | | |
| | ALRCLVSGVEEQRVRDGLPRLSNLSHQDEYAEL | | |
| | RSFCEEVRGRHAIPCEGPAKTTPAVSSASPPGAT | | |
| | ACFAPYVTLLRSDASRCYDNLPQGRVLAAVRSL | | |
| | VKHDAYRVLRFTVIHAVDSEATCKGGCLLRRTF | | |
| | TTRTIPCAEAECGFLARIPRGHIYWEEEGRTPTG | | |
| | PHTSAAVSRTTDASSRCGANLISGAAVRALLSE | | |
| | HIRHHLVVVSGGSLFEQRVGILQGSPVAMLLCD | | |
| | RLFSNVVDTALSSILSEHAERSLLLRRVDDVLVA | | |
| | TTSPAAAERCLRAMQRGWPSVGYLSNPSKLTLS | | |
| | KACGSLVPWCGLLLHDTTLEVSVEWRRIGVLL | | |
| | GSLRVGDPHYVHRGDYEPLYLTQRFLAVLQLR | | |
| | VAPTALCGRMNSKTRQLQTFYEVGLLWTRVVL | | |
| | EKVQEALPVARNCCVTVLLLRPLAVCVGRLCR | | |
| | LLSRHQRFLAARQSACDVSAAEVRACVLTALH | | |
| | RTVQAKLRVLQARTVRAMTAQSGRTQRGPSLK | | |
| | GRRNNFCSTKASSVGRKGCEQRRRGRNRRNTR | | |
| | VCLRSFWWLTAAEVESQWRRSLGALYRAAPRP | | |
| | GEACGSTASSPSPAASASLLMEDGPLSMHARAL | | |
| | SATRLSQT | | |
| 8075 | GKKRKRPVKEPVKDDPICRAKSSPQQPAGNTAR | EAA59961 | Aspergillus |
| | SLSTNRNQAADAGKICHPVISLYYRHVVTLRQY | | nidulans |
| | ILQRIPRSSRARRRRIAAVGGHSAARDGLAAVP | | FGSCA4 |
| | KNEKDLADLLDTTLVGILKELPPTRSEERRRDFI | | |
| | AFTQAQQTTQTGTDSGPIVDFAISSIFNRPSHGK | | |
| | LENVLSHGYRQGGGRLPCSIPNVAAQFPNKNVQ | | |
| | MLKQSPWTEVLALLGSNGDEIMLKLLLDCGLF | | |
| | MAVDARKGVYCQISGQAISSLKPIDTSPEDCPA | | |
| | AFNGSSSPVKRHAVPWQGAAPRAHTKGPKENQ | | |
| | QQLSPNAIIFCRQRMLYARPHLNANGGITFGLPN | | |
| | HVLSRFHSAKSLQQTVHVMKYVFPRQFGLHNV | | |
| | FTSHSSYYENPLSKNYSSREEEIARKEGLEAARN | | |
| | QLRKSGFHAAIGERQESIKIPKRLRGKPLELIRQL | | |
| | QNRSRRCCYKKLLQYYCSEELSGPWILGQLSAE | | |
| | SNSVLSTSSSRPLVTQPSLAHQDMQRELRPTSYA | | |
| | KGFSKGSGATKPKENLTDHATPAASVSAFCRAV | | |
| | IRNLIPLEFFGVGEQGITHQKMILGHVDRFIRMR | | |
| | RFESLSLHEVCEGIKPESLRQAPSENNISASDLQK | | |
| | RRELFHEFLYYLFDSILLPLIRGSFYVTESQVHRY | | |
| | RLFYFRHDVWRRLTAQPLAHLRASIFEELAPET | | |
| | AEKLLSGKKSIGYGSLRLLPKTTGIRPILNLRRRT | | |
| | LVRSIYAGKNRYHPAQSVNSAIAPVYSMLNYER | | |
| | GRRNDLLGSSMFSVGDMHSRLKKFKESLMSRG | | |
| | WDQRKRLYFVKLDIQSCFDTIPQAKIVRLVEKL | | |
| | VSEENYHWMKYVEMRLASEFDNMWPLRKPQQ | | |
| | RRTWSKYLQRVGPVGRPENLADAIANGSVVGR | | |
| | RNTVLVDTIAQKEYNGEGLLDILNEHIRNNLVKI | | |
| | GKKYFRQRKGIPQGSVLSSLLCSLLYAEMERDV | | |
| | LGFLQTDDALLLRLLDDFLLVTLDSGLAMDFLR | | |
| | VMVRGQPDYGISVNPAKSLVNFAAVVDGAQIP | | |
| | RLVDTPLFPYCGSLIDTRTLEIFRDQDRMLEGAD | | |
| | SASVALSDSLSIDSTRTPGRSFYRKVLASIKQSM | | |
| | HPMYLDSTHNSLPAVLLNVYKSFVTAAMKMY | | |
| | RYTRSLPGRARPRPEVVVRTIHDATQLGYRLIR | | |
| | GRHGLCRVTHPQLQYLGGAAFQFVLGRKQTQY | | |
| | AGVLRWLDGTLAEARQSVGNSVLLAQAVQKG | | |
| | NRTYREWRF | | |
| 8076 | PITRSTGRGRIETEQSPPSETTATTQSMWTETTA | S33901 | Silkworm Pao |
| | NTVMSLVAPTTESSCATANTEATTKLAEKPGNS | | |
| | KTEAVKQYIAKQNDVPTKQRAGTVKSDRSRNR | | |
| | KEQKIAKAREELARLQVELAAARLATLEAGSD | | |
| | DENSESEYSKSELDERVGTWLETQPTKTENHDR | | |
| | HKETPAGACDKQDFSDLTAAITLAVKAAREPRY | | |
| | TELPFFNGNHQDWLSFRAAYHETMNSFTKTENI | | |
| | NRLRRNLKGRAKEAVDGLLITNADPSDVIRSLE | | |
| | ARFGRPETIAITELDTLRALPRLTETPRDICIFSSK | | |
| | VTNAVATLRALNCTHYLYNPETTKTMLEKLTP | | |
| | TLRYRYYDFTAVQPKEDPDLIKFEKFMKREAEL | | |
| | CSPYAQPEQAGHYSQPAQHNRRTQNVHIVSEKP | | |
| | SRAKCPVCSNTEHTTTDCYIFKKADSNTRWDIA | | |
| | KNKHLCFRCLQYKNKTHNCKPKTCGINDCKYT | | |
| | HNKMLHFDRKIEKTDNSDKETTENINSAWTGK | | |
| | QKQSYLKIIPVQVQGPIGTVDTYALLDDGSTVTL | | |
| | IDEIICKKTGTTGPIDPLHIQAINNIKSTETRSRRV | | |
| | NLTLRGLNSRKEIIQARTVNDLQVTAQKIPKEQI | | |
| | DEYSHLQDISDIITYENAKPGILIGQDNWHMLLA | | |
| | SKVRRGNRNQPIASLTPLGWVLHGGRTRTLSHH | | |
| | INYINHASETQEDDKIENLVKQYFAMDALCITPR | | |
| | RPKTDPEEQALRILNSNTVHTTDGRYETALLWK | | |
| | TDNVSLPDNYNNSLKRLINIENKLDHNPELKQK | | |
| | YTEQMEALVAKGYAEPAPKTKTENRTWYLPHF | | |
| | AVVNPPKPEKLRVVHDAAARTRGVALNDMLL | | |
| | KGPNLLQSLPGVIMRFRQHNITATADIKEMFIQV | | |
| | KLRPEDKDALRYLWRKDQRDNKPPEENRMTSL | | |
| | IFGASSSPSTAIYVKNLNAQKHEATHPEAAATIQ | | |
| | NRHYVDDYLDIFKGLKDAVLVTTDFRRKHERK | | |
| | PTSKTFWIDSEIVLRWTRTESRSYKPYVAQRLTA | | |
| | IEDSSTINERRWLPTKHNVADDVTRHVPMSYQN | | |
| | EHRWFRWTEFLRQRQNSWPTESASETTEPMGE | | |
| | VNIAAAVPAGASWPRRRHEKWKCQPRNTRMR | | |
| | GKSDSDISRSRQRGAHRRHQNQGWSSTETSTKT | | |
| | TDPAHRRRPSCTEKNATDSHGGSNVQDEIGFFIV | | |
| 8077 | PAADKRVKMFNLKRVEIMNTLQDFEEFTKSFD | CAJ14165 | Anopheles |
| | ATIDAYQIPSRLEQLEELVSEFTELRKAFNETVD | | gambiae |
| | DSEAFDIMQKDRREFNKRSHEVRAFLLKNSSHS | | |
| | GASSGLNTTQVNTTISAGTQNHLRLPKVDLPSF | | |
| | DGEITKWLTFKDRFSSMVHDSTEMPEVLKLQYL | | |
| | LSALKGDAAHQFEHMQITADNYYVTWEALLKR | | |
| | YDNSKVLKREYFKAFYSLEKMKTDSTEELARIV | | |
| | NEANRLVRGLERLNEPVDKWDTPLTSLLFYKL | | |
| | DSKTLVAWEQYSVDFKTDEFTNLVEFLEQRVNI | | |
| | LKSSAQNICNQYSANSIMVTGRQARRDGRNVA | | |
| | LPVQQTNNTFKGYLKCPLCNEQHPLHVCERFER | | |
| | ASVINREEIVRKHGLCFNCLRKGHSARECRSTY | | |
| | VCQQCKRKHHSKLCKIGRLSEVEVVPSTSRLTA | | |
| | TAQANCSKKTVILSTAQIIILDVNDQPYKVRALL | | |
| | DNGSQLNFITERVAQELRLKRARVSEQIAGVGG | | |
| | AIMRVAGSVVGTIRSLTTEYTTCLEFLILPKIATD | | |
| | LPSETMDVRGWKLPKDVRLADPTFHERGSIDM | | |
| | LIGADTFVEMIKAKKIKLDHELPTLLETELGWIV | | |
| | SGAYKHNNLNQSMACTIVSQGGENDIASLMNT | | |
| | FFNIEEVQDQNLWNVEERECEDHFQATTRRDEN | | |
| | GRYVVRLPLKAERELGESKEVALRRLIGLERRF | | |
| | EREPKVKEAYEAFMQEYITLGHMSVRENENSSD | | |
| | GYYMPHHAVFKQDSTTTKCRVVFDGSCKTSNG | | |
| | RSLNDILKVGPTIQQDTTDILLRWRRRAIAVVGD | | |
| | VEKMYRQVWVHEEDRKFQRILWRSHSSEKIKT | | |
| | YELNTITYGTASAPFLAIRTLNQVLEDNKEKYPL | | |
| | AASRINDFYVDDFISGADSENEAKQLCEETKAA | | |
| | LAMGGFPLRKWASNCPHILPSETEIDNIQRVIEL | | |
| | KSREGAVSTLGLVWNPILDTLGVKISEPETCEIY | | |
| | TKRSIIRTIAKIYDPLGIVDTVKAKAKQFMQRV | | |
| | WSLKKENGDSYGWDEEIPQQMRQEWEVFERQ | | |
| | LTHLQEVQVPRCVTIVGARNIQIHGFCDASEEG | | |
| | YGACVYVRSTNGEEIVSRLFVSKSKVTPLATKH | | |
| | TIARLELCAAHLLGKLLVKLKRATEDPYETFCW | | |
| | TDSSTVIYWLKSSPSRWKTFVANRVSQIQNATK | | |
| | EFEWRHVPGIHNPADAVSRGRNPAEVVEDKLW | | |
| | WHGPDWLVKDPKHWPKNIESGNTCETAKEEK | | |
| | QTKTTLTCMVKEESFINKLCERVGSFTKLKRIVA | | |
| | YCHRFFDRKRIHRKSYFELRELKRAEKTIIRLVQ | | |
| | NEVYATEYECIKQGQQVVRKSPLRVIRPILDKD | | |
| | NVMRVGGRLSNADIKDEQKHPVIIPGKHRIAELI | | |
| | ADKYHKILRHAGAQLMINTMQLRFWIVGARNV | | |
| | AKRTVFNCVKCTRCRPKLIQQPMADLPEQRVR | | |
| | QARPFSISGVDYAGPIMVKGTHRRAVPTKGYISI | | |
| | FVCFVTKAVHIELVSNLTSSAFLAALRRFVARR | | |
| | GHVTELHSDNGTNFRGANNKLRELYKLLNSDT | | |
| | HQDEVVGWCAERDMKWKFTPPAAPHFGGLWE | | |
| | AAVKSMKFHLKRVLGTGHLTFEDLSTLLAEIEA | | |
| | CLNSRPITAISEDPNDMEALTPGHFLVGNHLQTV | | |
| | ADVDIADVPTNRLNHWRLIQKHMQHIWNRWH | | |
| | REYLSTLQKRAKWNKNAISIEPGRLVILQEDNV | | |
| | AVSKWPMARVVDLHPGKDGVTRVVTLKCANG | | |
| | KEIRRPIHRIAPLPIES | | |
| 8078 | TLGSSRSPSPGRRRHASEGGTAVPPTPANCGKPT | T26836 | Caenorhabditis |
| | KKRTRGQVSLATRIVGPLKRRINHKVDAAKRIL | | elegans |
| | AETEAKMEILLNMPQDQLVSSEDSTYLDALLIR | | |
| | LQTILVALEGMRDLISDKFRDTEMMVDPNRHQ | | |
| | HHQEVLDYLEKSSTARFVDHLTHDIQQLETEMR | | |
| | SRNIPITHFDPSLLATTDVETGATTEDDANDEER | | |
| | RDIEATIEDHAQNNGPSDHRVISDLRTPHGSTPS | | |
| | TGTPRLSSPGMVWDNEGLSLHDELQIANLLDPA | | |
| | NPQRSPMAPAAPTSSAAAPTQSAAAPPSSATAP | | |
| | MSSAAAYHLHGQHGQQLGAPAHTNGGTTHRQ | | |
| | QPLEQRIQTTTKGVLQGKREQLKQGPTETPLLV | | |
| | QHAPTTTGPGTRITPQRVLNAPALQTHQVINSQ | | |
| | VGYPSAGAHEYSAFHPLLQYAPPRGEDTLTHRL | | |
| | LAAIEAIATSQSQMQSELISQGRSVHVLTDRME | | |
| | ATEKLIVEPKILNTTVAEETSRPAMPQPTHETAQ | | |
| | QQQTTGSEYEYEFDSDDDNNQQPLPPQPRTEIR | | |
| | YVEVKNNGSHDTQNLLKYLGKYDGNSNIDSFL | | |
| | TDFKESVMENENLNQANKFMILKTHLLGKARD | | |
| | CISRDHVTAKALEKTITSLKSVFGKDENKTSLLA | | |
| | QIHAIGFPQSDVREMRRAIAKHSILVEQLVNSGL | | |
| | AANDERTFTPLTSRLPPAIRTRVTQFWGSKGEN | | |
| | ATFQEIFDYVTTCVDDMARESILALRHLPTAESE | | |
| | TEVGPLGIPYSGQINHANATTQNQGNPNGKKTS | | |
| | ISLADKPVYKREDHPKTYYDSNTGESLPGYNAP | | |
| | GKQGPVLRLLPRTFPLYEGTTKKTCKACKGSHH | | |
| | TLRCTLSSKDFRQALNASRLCPICTGYHSVEQCR | | |
| | CLMKCILCQGLHHTGGTLSPTSAIPTINPLLTFLP | | |
| | TFSDSAHFEITSTNIYNGRRIDMILGNDLLAWLN | | |
| | ANPETKKHILPSGRLVEITDFGHIVHPVPDKTIY | | |
| | QNHTQIEVTSETFMHASALINGPNPEDPNLALTL | | |
| | QVEQQWKLENIGIEAQPLNDHTTTSAKDLQASF | | |
| | ENTLRYTPEGILEVAFPLNGNEVRLKDNYEVAV | | |
| | KRLHATVNALKNSKNPNLLKQYDEIFKTQEASG | | |
| | IIESVTPNMKLETKYNYNMPHRAVIKESSNTTK | | |
| | VRVVYDASSHAVGQLSLNDVVHAGANMVIPLF | | |
| | GILIRSRFIKLMIVGDLEKAFHQVQVQPEFRNLT | | |
| | LFLWLTDLNKPITRDNICTKRFVRLPFGMSCSPN | | |
| | LLASTIVHFLVHNPDELNNDILDNLYVDNILIGT | | |
| | NDLALIMNRITRLKQIFSHMKMNIREFVVNHDE | | |
| | SMEKIDPKDRVSARTIKLLGMKWNSSPDADTYT | | |
| | IKIADVQTIMHPTKRDVASKMAETFDPLGLISPI | | |
| | QVSMKRLIQKLWSHEVNWKDPIPKHLLDDWQ | | |
| | AIQASFIDRTITVPRRLTTDFEYKDIQLLISSDASQ | | |
| | DIYAAAAYVYFSYGDDRPPVISLITSKNKIKPSR | | |
| | ETNWTIPKLELLGIEIGSNLASSIVKELRCKVTNI | | |
| | RLFTDSSCALYWILSKKNTRVWVANRIDQIHLN | | |
| | QTRMSECGIDTSIHHCPTKDNPADIATRGMSTSE | | |
| | LQNSDLWFNGPEFLKQKPEDWPCKIEGTFTCPA | | |
| | EFQAVVFAEILDPKTKKTKKPLMEKAEKPPASE | | |
| | TVLHILELPSKFESIISFRYTNSLRKLMLVTYRTL | | |
| | LAISKMRKGKVPTSWILEKFMMAPNLLEKRRV | | |
| | ARHYIFLQHYKECAEQGLTFPSSLRYYVAPDGL | | |
| | YRVLKQAKSPTLPAEANEPILVHPKHPLANLLM | | |
| | LETHEINGHLPEQYTRAALRTRYWLPNDSSVAR | | |
| | SVISKCIQCKKVHGLPFPYPHSMTLPESRTTPSTP | | |
| | FQNAGLDYMGPVEYSKDDGVSTGKAYVLIYTC | | |
| | FTTRATILRVVSDGSTERFIMALKTIFHQVGVPK | | |
| | MVYSDNAPAFILGGSILNDDISTWEHHSDPLTSF | | |
| | MATQSIHFFRITPVSPWQGGMYERIVGLVKHQI | | |
| | LKVCGADRFDYFTLSYIVSSAQAMVNNRPLMQ | | |
| | HSRQPDDMIAIRPCDFLNPGVMIETPPTEFTPSAP | | |
| | SGVPEQRVRAHLASLEETIELLWKYWSLGYIINL | | |
| | RQNHHRNVRCADLKPTVGQVVLVNTNLVKRQ | | |
| | NWPLGVIVQVNRSERTDEIRTAVVKCKGKLYK | | |
| | RSVCQLIPLEVQSSDMDSLPDTENREDGQECLM | | |
| | DAGMTVQHPSAPPLTIPSAALFDSPDEHYSPELF | | |
| | PRETCPNVTEATENPSPKIQNNTMIPLVPNTSTIQ | | |
| | NARLDLHERVGEVDNFENPDLDQVHVDSKDEV | | |
| | EYQDPSTTEELPTAIPGRSRPILPRRVKKPVYYN | | |
| | YFLHTTTAVTSTFSTPECCEMIPSPNYLVL | | |
| 8079 | EHLGVDPNPLHPIDQYTLLAANKILQRTKRYAE | NP_508646 | Caenorhabditis |
| | ALENLRHYVDDKFQEPVLRGSPLKDVYHEQVQ | | elegans |
| | EHLARLQPKSLLTEAKRDITMLERELLNHGFPIT | | |
| | TSDPQELVLTPYEYETSEDGTSSSDVDDLASFDG | | |
| | AFDNLRETMGSDHVQIDHQNPNPRVTIPSAILSP | | |
| | PTNGSSHLNYRTVSQPSPLTVHRDSALGSLSRQP | | |
| | SLADELQDERHQHRLSQIRLRALEDTFIARRQA | | |
| | DEEAEELGRQQLSQYREMRAARERRLEEMRSQ | | |
| | PSPPQAPAPAPRRPHTVHSGAEPTTPDLVPAPAL | | |
| | TGYPTPEQLLPQAMLQAMTEMGRLISQLQRDQ | | |
| | TQARREQTSFMNECREHLRPPAEGSIGQSAYSP | | |
| | DDEGEEQSQRGSSPPVQPIPDSRSPSGVINFETNA | | |
| | KNLPKFDGTGNFRAFRNGFDTVVLDDPRLPSVT | | |
| | KCNLLRNHLVGNAQQCISHDDDPLVAYQTTMD | | |
| | MLESVYGKGDTQRGLLERFRKLKFHQSNPEQM | | |
| | KLDLTSHQLLVQRLVSTGLSATDDRITMGLIGK | | |
| | LPISFRDKVTEFYTDMDDHPSAIAFYQRIRKHIN | | |
| | SFENGLIAASLQPLHVAPVNEIPSHYVKGSVHV | | |
| | VDQKQQPKKGELRHPTSSSGGQKERDTSAFYID | | |
| | PATGAQLGGHLRPGKRGVHLTLIARTFPLPDET | | |
| | SKKPCAACGGSHSPTRCHLTSQAFREATAQKGL | | |
| | CANCCGKHAIEQCKSHFTAPAFSDQDAHHLDSL | | |
| | EIDHLSISSQRTFDGKRIDMILGNDVLTCLHGDR | | |
| | HTRRHQLPSRRVVDDTRIGYIVHSVPSLILYTSD | | |
| | ERKWVFNDQNGLTHSLMLANMVLDHQYVEDP | | |
| | ELKLHWSIEQLWKFENLGIEPIPLVDETKKSTQD | | |
| | LLAEFQQNACYTNGVLEVALPENGNEEKLKNN | | |
| | YAIAYKRLCSLHETLTKGKNLITKYDRVIKDQL | | |
| | LAGIIELVTPEMKPDSPIEYFMPHRAVIKESSNTT | | |
| | KLRVVLDASSPIGKDLSLNDCLHAGTNLLTPLY | | |
| | GILLRSRCYRYIIVADIEKAFHQVRLQVKHRSVT | | |
| | QFLWLADPSQPANADNVVRYRFTRIPFGVASSP | | |
| | FLLGAAIHHFLGRNPHRLNNEIRDNLYVDNCML | | |
| | GTDDFTKVMPTAMAAKSIFRKMNMNLREFVTN | | |
| | CDGIMQHIRAEDRAESRDIKLLGCMWNSNETV | | |
| | DTYSIKIAVLDIDHPTKREVASKLAETFDPLGLV | | |
| | TPILVQFKRLIQQLWIAGVSWKDRIPIELLPLWR | | |
| | NLQKSFVDKSIHVERRLTFVNEEVIDCQLIIFTDA | | |
| | SQDIYAAAAYAHFTYKKWPPVTRLITSKSKIKE | | |
| | VSAANYTIPKLELLGILCGSNLAVTLSKELRLPIS | | |
| | SIKLFTDSSCALYWILSAKNTRAWVHNRVQKY | | |
| | HENCARMSECGLSTSLHHVPTKENPADLATRG | | |
| | MSTTELQKSLFWFRGPRFLANPPESWPQKIEGTI | | |
| | TCPAEFQDLVYKEIIDTSTEKKKSKPLIEKAIPAA | | |
| | PKATESVLHLTTGPFKSFIPTLNSVCKMFPGKSW | | |
| | DSEIMVEFKNSESALHRRKLVRKLIILHHYRESE | | |
| | ALGLKLPADLDYYVDSHGFYLVKKQVTSHALP | | |
| | QEANEPVILFKDHPLATLVMRETHVINGHSSEL | | |
| | YTVSAAKTMFWIPHIKVLAKSVVSNCVDCKKV | | |
| | HGLPFRYPNSKTLPEKRTSPSKPFATAGLDYMG | | |
| | PIEYLKDDGVTIGKSYVLVYTCLVTRGAMLRVL | | |
| | PDATTETYLMGLRSIFHCVGSPTDIYSDNAAIFK | | |
| | LGASMLNDDILSGDELSDSLTSYLASQQINFFYI | | |
| | TPLSPWQGGVYERIVGLLKHQLYKVSSVEKLS | | |
| | MFSLQYLVSGAQAMINSRPLTPHARSPNDMIAL | | |
| | RPIDFQLPGVMLDIPFVHPTGNGRGAEERARAH | | |
| | LAQLETALNRLWQIWTLGYLFHLRKAKHRNKK | | |
| | CTSIKPAVGQVVLIDTNHVNRHKWPLGVILQVH | | |
| | ESKRDHEVRTATVKAHGKRCLRSVCQLIPLEVQ | | |
| | ASEDFTSADPPSEGDLVELEEHDCDDPTSDIPTQ | | |
| | AYFEHSRSTARTLLRVSPRVSEIRCLSLGRVTDS | | |
| | PLLVPSVNNN | | |
| 8080 | FIGSIASNSSLTDCQRFHYLKSYLAGDALALVKH | AAB03640 | Drosophila |
| | IPVTNDNYREAWERLEQRYNKQSLIIRSFLNSFM | | melanogaster |
| | SLPSAINSNIGTVRKIADGADEVIRGLRALNCEE | | |
| | RDPWLIFILLSKLDSDTRQAWAQCAESEEKGVTI | | |
| | NRFLKFLTSRCDTLEAFELTRSTQARRAATTHH | | |
| | ADTHPRREEPKCTSCQQNHQLFKCPQFIALDIAS | | |
| | RRDFLKSRKLCFNCLSPAHMVGNCTSRHTCRIC | | |
| | RRKHHTLVHGSSQPIQNGNNIDTASVDSRDRPA | | |
| | VSHAGSTIGHNQPLAREGHRLGSETPAENNFTH | | |
| | HTLENIPAAGSQTLLPTILADVIDAWGNTTTCRL | | |
| | LLDTGSTITLASESFVQRIGVRRTHARISILGLAA | | |
| | NSAGVTRGRAHIKLRSRHSGQTVELVSFILTSLT | | |
| | SSLPAQVIDTSSSTWRQICELPLADPTFCTPGAID | | |
| | VIVGSDQLWSLYTGDRKHFGNDFPIALNTVFG | | |
| | WILAGSYSAFDDHPTSAVTHHADLDTMVRSFM | | |
| | EMDSIQPNQALLDASDPTERHFAATHKRSTDGV | | |
| | YVVEYPFKEKAPPIDSTLPQAINRFFSLERKFRR | | |
| | YPELKQQYEAFLDDYLQRGHMEKLTSAQVEES | | |
| | PDTCFYLPHHAVIKLDSLTTKCRVVFDGSGKDS | | |
| | SGVSLNDRLHIGPPIQRDLFGVCLRFRQHQYVL | | |
| | CADVEKMFRGIKVFKPHTNFQRIVWRTTENEPL | | |
| | LHFRLLTVTYGLAPSPFLAVRVLKQLADDHGHE | | |
| | YPAAAHALLHDAYVDDIPTGANTFEELMILKDE | | |
| | LIALLDKGKFKLRKWSSNSWRLLKSLPEEDRCF | | |
| | EPIQLLNKSAADSPVKVLGIQWNPGKDVLYLNL | | |
| | KGCDATISPTKRELLSQLSRIYDPLGLVAPVTVL | | |
| | LKLIFQESWTSVLQWDDPIPESLRTRWRALVED | | |
| | LPALTQCQVPRYIASPFRDVQLHGFADASSHAY | | |
| | GAVVYARVAVGCSFQVTLVAAKTRVAPIKPVSI | | |
| | PRLELNAALLLSRLLSIVKTSLTIPLESTSCWTDS | | |
| | EIVLHWLSAPPRRWNTYVCNRTSEILSDFPRSC | | |
| | WNHVRTEDNPADCASRGLHPSKLLEHRLWWK | | |
| | GPSWLATPTSEWPPSTSKFSVSSSFDVNTEERAI | | |
| | KPTTLHNFPDESIHELLIHKFSTWTRLIRVSSYCH | | |
| | RFIHTLRSHHRNSAPFLTSEELLDAQRRLIRHVQ | | |
| | QKSFAREYEQLENRRQLNAKSHLIRFSPFLDDY | | |
| | GVMRVGGRIEQSTLNYNAKHPILIPKDTPLAGL | | |
| | LVRHFHVSYLHTGVDATFTNLRQQYWILGARN | | |
| | LVRKAVFQCKSCFLQRKGTSNQIMGELPIPRVQ | | |
| | ASRCFQHTGLDYAGPIAIKESKGRTPRIGKAWFS | | |
| | IFVCLTTKALHIEVVSELTTQAFIAAFQRFIARRA | | |
| | KPTDLYSDNGTTFHGGKKTLDDMRRLAIQQAK | | |
| | DEELAGFFANEGISWHFIPPSAPHFGGMWEAGV | | |
| | RSIKLHMKRILGSKALTFEELSTVLTQIEAILNSR | | |
| | PLCPTGDNSLDPLTPAHFLTGSPYTALPEPCRLD | | |
| | MQVNRLERWNQLQAMVQGFWKRWHMEYLTS | | |
| | LHERTKWHLETENLKIDTLVVLKEPNLPPSKWI | | |
| | LGRITAVHAGIDNKVRVVTVKTAHGLYKRPIAK | | |
| | IAVLPLC | | |
| 8081 | VRGAGVRSRGRGRGRVLKGTGESDGHSAKVEQ | CAB78181 | Arabidopsis |
| | SVGSQPEFVEPGVRNGLGADIAGATGVGAGGA | | thaliana |
| | GVGTGVHAVGAEGPGVMGAAAGGAQVPEVGL | | |
| | AGLLRQLLERLPGVVPVETPVAPRVAEVQQRA | | |
| | AVAEEVLSYLRMMEQLQRIDTGYFSGGTSPEEA | | |
| | DSWRSRVGRNFGSSRCPAEYRVDLAVHFLEGD | | |
| | AHLWWRSVTARRRQTDMSWADFVAEFKAKYF | | |
| | PQEALDPYAGQGMEDDQAQMRRFLRGLRPDLR | | |
| | VRCRVSQYATKAALVETAAEVEEDFQRQVVGV | | |
| | SPVVQPKKTQQQVTPSKGGKPAQGQKRKWDH | | |
| | PSRAGQGGRARCFSCGSLDHKDAGGQFLAVLG | | |
| | RAKGVDIQIAGESMPADLIISPVELYDVILGMD | | |
| | WLDYYRVHLDWHRGRVFFERPEGRLVYQGVR | | |
| | PISGSLVISAVQAEKMIEKGCEAYLVTISMPESV | | |
| | GQVAVSDIRVVQEFQDVFQSLQGLPPSQSDPFTI | | |
| | ELEPGTAPLSKAPYRMAPAEMAELKKQLKDLL | | |
| | GKGFIRPSTSPWGAPVLFVKKKDGSFRLCIDYRE | | |
| | LNRVTVKNRYPLPRIDELLDQLRGATCFSKIDLT | | |
| | SGYHQIPIAEADVRKTAFRTRYGHFEFVVMPFG | | |
| | LTNAPAVFMRLMNSVFQEFLDEFVIIFIDDILVY | | |
| | SKSPEEQEVHLRRVMEKLREQKLFAKLSKCSFW | | |
| | QREMGFLGHIVSAEGVSVDPEKIEAIRDWPRPT | | |
| | NATEIRSFLGWAGYYRRFVKGFASMAQPMTKL | | |
| | TGKDVPFVWSQECEEGFVSLKEMLTSTPVLALP | | |
| | EHGQPYMVYTDASRVGLGCVLMQHGKVIAYA | | |
| | SRQLMKHEGNYPTHDLEMAAVIFALKIWRSYL | | |
| | YGGKVQVFTDHKSLKYIFTQPELNLRQRRWME | | |
| | LVADYDLEIAYHPGKANVVVDALSRKRVGAAL | | |
| | GQSVEVLVSEIGALRLCAVAREPLGLEAVDRAD | | |
| | LLTRVRLAQKKDEGLRATKMYRDLKRYYQWV | | |
| | GMKMDVANWVAECDVCQLVKAEHQVLGGML | | |
| | QSLPIPEWKWDFITMDLVVGLRVSRTKDAIWVI | | |
| | VDRLTKSAHFLAIRKTDGAAVLAKKFVSEIVKL | | |
| | HGVPLNMKEAQDRQRSYADKRRRELEFEVGDR | | |
| | VYLKMAMLRGPNRSISETKLSPRYMGPFKIVER | | |
| | VEPVAYRLELPDVMRAFHKVFHVSMLRKCLHK | | |
| | DDEALAKIPEDLQPNMTLEARPVRVLERRIKEL | | |
| | RQKKIPLIKVLWDCDGVTEETWEPEARMKARF | | |
| | KKWFEKQVAA | | |
| 8082 | VEVLEEEVEVQTLTPSRSEGASGSRNPRHRRRG | AAM08509 | Oryza sativa |
| | SRTPPLSDPLRREAGGALLRHPPVNVEPEAPVQ | | Japonica Group |
| | RWLDDVANLVTTAQRRLAVSGRSTATGTSRTS | | |
| | TTLSSSARRRARRIATASRRSTAPTSSGASESRR | | |
| | RHDSLYGEQDARVNIERRRDERRATRMGEGAS | | |
| | SSGVPRFSSRGGPPLTSTPGGTGYKAFVASLRNV | | |
| | RWPPKFCLNLTEKYNGSINPSEFLQIYTTIIVAAG | | |
| | GDDRVMANYFPMALKAHAVVYAFWNGVRHN | | |
| | RKLEKIASKEPKTTAELFELADKVAQKEEAWA | | |
| | WNSPSTGAAAAAAPETAPRSKRRDRRGKRKPA | | |
| | RSDDEGHVLAADGPSRAPRRERATDGKTSYTA | | |
| | PSGKGRSADKWCSVHNTYRHSLADCRSVKNLA | | |
| | ERFRKADEEKRQSRREGKALTTPANDQREESKK | | |
| | KAPADDGDDSEGLEFQDILCKVLRDKCGQKSA | | |
| | AKTCGILEFVRDNSRVRSQKAVFLMAEKPPPSP | | |
| | SSASPGSVKEKIQQLDLSEVNEGNVMTITLDKLT | | |
| | PDQKEFEAMMQQARNQFLNSFMQTRKGTVVQ | | |
| | KYQVRVVADVPGTGSSKDGEMKQALGGSAQP | | |
| | SNKGATNGSAQENQGDHSQGVHGVQGDGTQG | | |
| | PRGGSLNQDGSASQEFFNNFQDRVDYAVHNAFI | | |
| | NQSGVLVNTLSNMMKSIADGSIAKHQAAGPVY | | |
| | LPGGQLVNPRQLMRENPQHSGQVANRLTQDQV | | |
| | ATMFLPLQPTVDLVQQQPIQQTPPIQQVVQPIQQ | | |
| | QVVHWADLEKQFHSYFYSGVHEMKLYNLTAIK | | |
| | QRHDEPVHEYIQRFREMRNKFISLSLTDAQIADL | | |
| | AFQGMIAPIREKFSSEDFESLPHLTQKVTLHEQR | | |
| | FAEARRNSRKVNHVCSYMCGSDDEDDDSEIAA | | |
| | AEWVRSKKVMPCQWVKNSGKEERYDFDITKA | | |
| | DKIFDLLLWEMQIQLPAGHTIPSAEELGKKRYC | | |
| | KWHNSGSHTTNDGKVFRQQIQSAIEGGKIKFDD | | |
| | SKKPMKVDGNPFPVNMVHTAGQTADGGRARG | | |
| | FQMNSAKIINKYQRKYNKQQEKHYEEGDDGFD | | |
| | PHWGCEFFRFCWNEGMRLPYIEDYPGCEQAVF | | |
| | KKPEGAENRHLKPLYINDYVNGKPMSKMMVD | | |
| | GGAALNLMLYATFRKLGRNAEDLIKTNMVLKD | | |
| | FGGNPSETKGVLNVELTVGGKTIPTTFFVIDGKG | | |
| | SYSLLLGRDWVHANCCIPSTMHQCLIQWQGDKI | | |
| | EIVPADSQLKMENPSYYFEGIVEGSNVYTKDTV | | |
| | DDLDDKQGQGFMSADDLEDIDIGPGDRPRPTFIS | | |
| | QNLSSEFRTKLIELLKEFRDCFAWQYYEMPGLS | | |
| | RSIVEHRLPTEPGVRPHQQPPRRCKADMLEPVK | | |
| | AEIKCLYDASFIRRCRYAEWVSSIVPVIKKNGKE | | |
| | RVCIDFRDLNKATPKDEYPMPVADQLVDAASG | | |
| | YKILSFMDGNVGYNQIFMAEEDIHKTAFRCPSAI | | |
| | GLFEWVVMTFGLKSAGATYQRAMNYIYHDLIS | | |
| | WLVEVYIDDVVVKSKEIEDHIADLRKVFERTRK | | |
| | YGLKMNPTKCAFGVSAGQFLGFLVHERGIEITQ | | |
| | RSINVIKMIKPPEDKTELQEMIGKINFVRRFISNL | | |
| | SGRLEPFTPLLRLKADQQSTWGAEQQKALDNIK | | |
| | EYLSSPPVLIPPQKGIPFWLYLSAGDKSIGSVFIQ | | |
| | KLEGKERADVVKYMLSAPILKGRIGKWIFSLTE | | |
| | FDLWYESQKAIKGQAIANFIVDHRDDSIGLVEV | | |
| | VLWTLFFDGSVCTHGCGIGLVIISPRGACFEFAY | | |
| | TIKPYATNNQAEYEAVLKGLQLLKEVQADTIEI | | |
| | MGDSLLVISQLAGEYECMNDTLIVYNDKCQEL | | |
| | MKEFRLVTLKHFVQEHIIYRFGIPQTVTTDQGSI | | |
| | FVSDEFVQFADSMGIKLLNSSPYYTQANGQAEA | | |
| | SNKSLIKLIKRKISDYPRQWHTRLAEALWSYRM | | |
| | ACHGSTQVPPYKLVYGHEAVLPWEVRIDSRRTE | | |
| | LQNDLTADEYYNLMADEREDLVQSRLRALAKV | | |
| | TKDKERVAWHYNKKVVPKDFSEGELVWKLILS | | |
| | IGTRDSKFSKWSPNWEGPFQIHKVVSKGAYML | | |
| | QGLDGEVYGRALNGKYLKKYYPSVWVNA | | |
| 8083 | LHDDLQRGPSIIKNTPPPFVIQFGSLPPVTFFEYG | BAB08213 | Oryza sativa |
| | SKVYMQQAQDVTQFQEAQSKKORKRASAKAK | | Japonica Group |
| | KERRTLMLEARTLLKESVVAEIKGDIQAAQKLR | | |
| | VKASNRRSITASLRAPDPVATPKLPTPTVQHTEV | | |
| | ELLEALEAVSDNLRRHISHTRRANSPHPLRNYR | | |
| | RKYRKVQRLHQLVSSRIAQSSLLEEDWSLDTSV | | |
| | LIKKVFKFPSILEPPYDLFPDEWACEPTKIKEKVR | | |
| | CAIMKEYWKRRDREHLVLPGSTIFVDYNTYTPR | | |
| | QQSTWESCLHLSAVGGSSDYNNNRFAVLRSEA | | |
| | PAPRSEDLRQELRELQDRMAQLGRRLQDHEAP | | |
| | RSSSTQAGGRSRRYQPSYHPQHDRRTLAPRRTL | | |
| | PSTQVMHQRQTALPPRWNRWSRHQDYPTSSRL | | |
| | AQEWRVREAPSSQVPPHVPSSPRREVYTQRRRE | | |
| | TNAPNPATRQVAPPLLPTPSIPPRRQHAPTENQR | | |
| | KRERRRNNRYALYRELEDLVLKHTQVRVRPDG | | |
| | EVHQEDERIVFRISPSLERDARYNYLIARLTPKP | | |
| | RRTLDVADKNREQALTQPCPVTILQRGKGPVQ | | |
| | ATLGISLSTSARQSKENQSTPMEGVEQTPVEQV | | |
| | DKASRQEEAIINPMVDVLPQQESSSVPPARVEQ | | |
| | VAGSKNIEDPKESIVMCSALAAHYETKPNAAW | | |
| | VPPPVTHDFTYPSDEEIVPNPRANFSKTFLPQLD | | |
| | QVASRPGANTRMKAIAIKNVEATPSQARKDLED | | |
| | HVEVEDLDELESTSSSSLEVNLNLPRYNELNPSL | | |
| | PSDGEGYPNNFDSAPAHVTAEGDPRQHARQHA | | |
| | PRGENPSIGNWATMKEVFKKHFVAMKKDFSIV | | |
| | ELSQVRQWRDEAIDDYVIRFRNSFVCLAREMHL | | |
| | EDAIEMCVHGMQQHWSLEVSRREPKTFSALSS | | |
| | AVAATKLEFEKSPQIMELYKNASAFDPTKRFNA | | |
| | TKPSGSGNKPKVPTEANSTKVFSTAPQGQVPMI | | |
| | GAKNEQVGGRQRSTLQDLLKKQYIFRRELVKD | | |
| | MFNQLMEHRALNLPEPRRPDQVTMTDNPLYCP | | |
| | YHRYIGHAIEDCIAFKEWLQRAVNEKRINLDAD | | |
| | AINPDYHAVNMVSVEPFPQKQREGRRATSWAP | | |
| | LAQVEDQIAKIMLTKAPATHVEASHGDNNRAW | | |
| | SIVRWKPQPMSFPPRRPQMKLSPHTHPTSRRWL | | |
| | DPSRRRPPPRFVPFSEGDESFPRRGRELPTLAQFL | | |
| | PKGWEQSSTSTREAKGVNNSIPTPDIAPCNVILT | | |
| | YNDSTSTGSDETFTGREREIFHAELDPEKTKVEE | | |
| | VNISLRGGKTLPDPHKSKVPNVDKPAKKASPPG | | |
| | EAPEAPETKTGSKEKPAVDYKVLAHLKRIPALL | | |
| | SVYDALMMVPDLREALIKALQAPEVYEVDMA | | |
| | KHRLYDNPLFVNEITFADEDNIIKGGDHNRPLYI | | |
| | EGNIGSAHLRRILIDLGSAVNILPVRSLTRAGFTT | | |
| | KDLEPIDVVICGFDNQGKPTLGAITIKIQMSTFSF | | |
| | KVRFFVIEANTSYSALLGRPWIHKYRVVPSTLH | | |
| | QCLKFLDGNGVQQRITSNFSPYTIQESYHADAK | | |
| | YYFPVEENKQQLGRTTPAADIIVEPGTETTPEHV | | |
| | YPIYYTNIAQSKTLYLNTDHLGGNFSRKRETAQ | | |
| | KQRRCANYHHLTGKTESKQGSRACTTGSGQEE | | |
| | SCRDRGGKSGCSVHAAPSPLHFSFYPAREEACT | | |
| | TMKAEPRMARLLEKAGINLQRNNRLPPPPAVCE | | |
| | DWWAQAEEFIKRRCKEQPKYGLGYINVDEPDD | | |
| | EDEVFEDDIFHCCTISTTTRGDALLQQHPFEVAA | | |
| | VGVEEELDVAGALKOLDDGGQPTIDELVEMNL | | |
| | GTEDDPRPIFVSGMLTEEEREDYRSFLMEFRDCF | | |
| | AWTYKEMPGLDSRVATHKLAIDPQFRPVKQPP | | |
| | RRLRPEFQDQVIAEVDRLINVGFIKEIQYPRWLA | | |
| | NIVPVEKKNGQVRVCVDFRDLNRACPKDDFPLP | | |
| | ITEMVVDSTTGYGALSGYNQIKMDLLDAFDTAF | | |
| | RTPKGNFYYTVMPFGLKNAGATYQRAMQFVL | | |
| | DDLIHHSVECYVDDMVVKTKDHEHHQEDLRIV | | |
| | FERLRRHQLKMNPLKCAFAVQSGVFLGFVIRHR | | |
| | GIEIEPKKIKAILNMPPPQELKDLRKLQGKLAYIR | | |
| | RFISNLSGRIQPFSKLMKKGTPFVWDEECQNGF | | |
| | DSIKRYLLNPPVLAAPVKGRPLILYIATQPASIGA | | |
| | LLAQHNDEGKEVACYYLSRTMVGAEQNYSPIE | | |
| | KLCLALIFALKKLRHYMLAHQIQLIARADPIRYV | | |
| | LSQPVLTGRLGKWALLMMEYDITFVPQKAIKG | | |
| | QALAEFLATHPMPDDSPLIANLPDEEIFTAELQE | | |
| | QWELYFDGASRKDINPDGTPRRRAGAGLVFKT | | |
| | PQGGVIYHSFSLLKEECSNNEAEYEALIFGLLLA | | |
| | LSMEVRSLRAHGDSRLIIRQINNIYEVRKPELVP | | |
| | YYTVARRLMDKFEHIEVIHVPRSKNAPADALAK | | |
| | LAAALVFQGDNPAQIVVEERWLLPAVLELIPEE | | |
| | VNIIITNSAEEEDWRQPFLDYFKHGSLPEDPVER | | |
| | RQLQRRLPSYIYKAGVLYKRSYGQEVLLRCVD | | |
| | RSEANRVLQEVHHGVCGGHQSGPKMYHSIRLV | | |
| | GYYWPGIMADCLKTAKTCHGCQIHDNFKHQPP | | |
| | APLHPTVPSWPFDAWGIDVIGLINPPSSRGHRFIL | | |
| | TATDYFSKWAEAVPLREVKSSDVINFLERHIIYR | | |
| | FGVPHRITSDNAKAFKSQKIYRFMEKYKIKWNY | | |
| | STGYYPQANGMAEAFNKTLGKILKKTVDKHRR | | |
| | DWHDRLYEALWAYRVTVRTPTQATPYSLVYG | | |
| | NEAVLPLEIQLPSLRVAIHDELTKDEQIRLRFQEL | | |
| | DAVEEERLGALQNLELYRQNMVRAYDKLVKQ | | |
| | RVFRKGELVLVLRRPIVVTHKMKGKFEPKWEG | | |
| | PYVIEQAYDGGAYQLIDHQGSQPMPPINGRFLK | | |
| | KYFV | | |
| 8084 | TPVDSMSKDPPAEAENGISTTSEPEKDPNAAKSC | AAX95475 | Oryza sativa |
| | PSDKKHEPTRTTSEVTRTWCPIHKTRRHILQTCS | | Japonica Group |
| | VFLDVQAEIRASKERGIQRTSPPRDVYCPIHKAK | | |
| | THDLSSCKVHLSAMRTSPPKVQQSQIYPRDADK | | |
| | EQGATTISDRFVRVIDIDPHEPSILHLLEDQASSS | | |
| | TSTPCDVYAIDGTSTSRDGDAETADQSVTPTPA | | |
| | QHIRILNAILSESPFDPVLNADLDQWTERLRESV | | |
| | ANLSNAFAEAAARAPLEQPPTGGANGEQPEERT | | |
| | PHRQATPPPRGNSDLRDHLNGRREARRTQDNE | | |
| | NRTIEKYDGSTDPEEFLQVYSRVLYVAGADDN | | |
| | ALANYLPTAMKESAQSWLVHLPPYSISSWADL | | |
| | WQQFVTNFQGTYKRHAIEDDLHTLTNMIPEITD | | |
| | ASVIRALKSGVRDHYTTQELATRRITTAHKLFEI | | |
| | VDRCAHTDDALRHKNDKPRTGGEKKPAKDAR | | |
| | LSQARKRVAGVGNGRLKRSPRPECYTIHKSDKH | | |
| | PLETCFVFKKALTKQLALEKGKRGAASKMKWS | | |
| | EQKIEFSEADHLKTAVTPGRYPIVVEPTIQNIKV | | |
| | ARVLIDDGSSINLLFASTLDAMGIPRKQITFDVA | | |
| | EFDAAYNAIIGRTALTKFIAASHYAYQVLKMPG | | |
| | PKGTITIQGNEKLAVQCDKRSLDMVEHTPNTPA | | |
| | TAEPPKKPFDMPGVPREVIKHKLIVRPNAKPVK | | |
| | QKLRRFAPDRKQAIREAIRKWRMCIDFTGLNKA | | |
| | CPKDHFPLPRIDQLVDSTAGFQGALNDQLGHNV | | |
| | EAYVDDIVVKKKTSDSLIDDLRETFDNLRRYRL | | |
| | MLNPKKCTFGVPSGKLLGSLVSERGIEVNPEKIV | | |
| | GHREREIAHKTQGSPEANWMHGGTKQEAEDAF | | |
| | IALKHYLSNPHVLVAPQPNEELFLYIAATPYSPT | | |
| | VTAVSSFPLGEVVRNKDVVGRIAKWVLELSQF | | |
| | NVHFVPQIAIKSQVLADFVADWTMPENKSDSQT | | |
| | DSETWTMAFDGALNSQGPGAGSILTSPSGDQFK | | |
| | HEIHLNFRATNNTAEYEGLLAGIRAAAALGVKR | | |
| | LIVKGDSELVANQVHKDYKCSSPELSKYLAEVR | | |
| | KLEKRFDGIEVRDIYCKDNIEPDDLAWRASRRE | | |
| | PLEPSTFLDVLTKPSVKEANNEEAEKITRQAKIY | | |
| | CMIGNDLYKKASNGVLLKCLWSDDGKHLLLDI | | |
| | HEGICESHAGGRKLRCEACQFHSKHTKLPAQVL | | |
| | QTIPLTWPFSCWGLDILGPFPRGQGGYKFLFVAI | | |
| | DKFTKWTEVTPTGEIKANNAINFIKGIFCKYGLP | | |
| | HRIITDNCSQFISADFQDYCIKLGVKICFASVSHP | | |
| | QSNGQVERENGIVLQGIETRVYDRLMSYDKKW | | |
| | IEELPSILWAVCTTPTTSNKETSFFLVYGSEAML | | |
| | PTELRH | | |
| 8085 | TEKLPPSPGTGVKPPVNKTEAKNPSAEVDPSNIV | ABF96295 | Oryza sativa |
| | PITLDRLTAEQRDELEQMMSNVKNKFMDSFQE | | Japonica Group |
| | TRRETIPMRSARVHLKVTKVLQLAQVTKVTFLK | | |
| | MWADLEKQLHSYFYSGIHEMKLSDLTAIKQRH | | |
| | DESVQDYIQRFREMRNRCYSLSLTDSQLADLAF | | |
| | QVLIAPIKEKFSAQDFESLSHLAQKVTLHEQRFA | | |
| | EAKKNFKKINHVYPYCDSDDEDDDSEVAAAEW | | |
| | VKRKKVIPCQWVKSSGKEERFDFDITKADKIFDI | | |
| | LLREKQIQLPAGHIIPSAEELGKRRYCKWHNSGS | | |
| | HSTNDCKVFRQQIQAAIEGGKIKFDDSKKPMKV | | |
| | DGNPFLVNMVHTSERAADGGSNRKFQVNSARII | | |
| | SKYQRKYDRQQGEYHEEDGGFDPHWDCEFFRF | | |
| | CWNEGMRLPSIEDCPGCSNAGNSSRSYSRAEFE | | |
| | AKQADVDDVEEASAKVVLSPEQAIFEKPEGIEN | | |
| | RHLKPLYINGFANGKPMSKMMVDGGAAVNLM | | |
| | PYATFRKLGRNPNDLIKTNMVLKDFGGNPSETK | | |
| | GVLNVELTVGSPGDRPRPTFISKNLSSEFRTKLIE | | |
| | LLKEYRDCFAWEYYEMPGLSRSVVEHRLPIKPG | | |
| | IRPYQQPPRRCKADMLEVVKAEVKHLYDAGFI | | |
| | HPCRYAEWVSNIVPVIKKNGKVRVYIDDEVVIS | | |
| | KEIEDHIADLRKVFERTRKYGLKMNPTKCAFGK | | |
| | LEPFTPLLRLKADQKFTWGAEQQKALDNIKKYL | | |
| | SSPPVLIPPQKGISFRLYLSAGDKSIGSVLIQELER | | |
| | KERAIFYLSRRLLDAETRYSPVEKLCLCLYFSCT | | |
| | KLRHYLLSNECTVICKADVVKYMLSAPILKGRV | | |
| | GKWIFALTEFDLRYESPKTIKGQAIADFIMDHRD | | |
| | DSIGSVDIVPWTLFFDGSVCTHGCGIGLVIISPRG | | |
| | ASFEFAYTIKPYVTNNQAEYEAVLKGLQLLKEV | | |
| | EADAIEIMGDSLLVISQLAGEYECKNDTLMVYN | | |
| | EKCRELMSGFRLVTLKHVSREQNVEANDLAQG | | |
| | ASGYKPMLKDVEIEVATITADDWRYVVFQYLQ | | |
| | NPSQSASRKLCYKALKYTLLDDELYYRTIHGVL | | |
| | LKYLSADQAMVVMGEIPPYKLVYGHEAVLPWE | | |
| | VRIGSRRTYLQDELTTDEYYNLMADEREDLVQS | | |
| | RLRALAKVTKDKERVARHYNKKVVPKSFSEGE | | |
| | LVWKLILPIGTRDNKFGKWSPNWEGPFQIHKVV | | |
| | SKGAYMLKGLVGKVYGRALNGKYLKKYYPNV | | |
| | WVNL | | |
| 8086 | AAEEGAEPSASVAEDGEAQAPSQPPSAPAPSQPS | ABF96966 | Oryza sativa |
| | SAPATSVQVPNTADVAKAATAARALQTKAEILS | | Japonica Group |
| | TNQLVVPQAAPSQPAAPTALAVVQAQISLDPEA | | |
| | QAEADMEAMRQNMTRLQDMLRQMQEQQQAY | | |
| | EVTRWTKATSAPILQYSAGYAPPQVRPQVVTQP | | |
| | SPPLAAQPPVYFAGQHQPSGQATQTVAEGASAL | | |
| | QAQLQVFHRQLNQPHYISSTTPSAHPVPTIRQQV | | |
| | PTRGFGTNQAPIQAAMTWLQPIFDPSMAAQQVP | | |
| | PVGAGQPNAMAQLHAQAAISPFATPYPQQGAV | | |
| | NRAGGEKGLPLSGGIKTRPIPPQFKFPPVPRYSG | | |
| | ETDPKEFLSIYESAIEAAHGDENTKAKVIHLALD | | |
| | GIARSWYFNLPANSIYSWEQLRDVFVLNFRGTY | | |
| | EEPKTQQHLLGIRQRPGESIREYMRRFSQARCQ | | |
| | VQDIIEASVINAASAGLLEGELTRKIANKEPQTL | | |
| | EHLLRIIDGFARGEEDSKRRQAIQAEYDKASVA | | |
| | TAQAQAQVQIAEPPPLSVRQSQSAIQGQPPRQG | | |
| | QAPMTWRKFRTDRAGKAVMAVEEVQTLRKEF | | |
| | DALQASNHQQPARKKVRKDLYYTFHGRSSHTT | | |
| | EQCRNIRQRGNAQDPRLQQGTTVEAPREAVQE | | |
| | QTPPVEQRQDVQQRMGLPTQALTPAPTSLRGFG | | |
| | GEAVQVLGQTLLLIAFSSVENRREEQILFDVVNI | | |
| | PYNYNAIFGRATLNKFKAISHHNYLKLKMPGPK | | |
| | GVIVVKGLQPSAASKRDLAIINRAVHNVETEPH | | |
| | ERPKHTPKPTPHGKVAKVQIDDFDPTKLVSLRS | | |
| | PRLKLRKMSADRQEAAKAEIHMNPLNIPKTSFV | | |
| | TPFGTFCQLRMPFGLRNAGATFARLVYKVLGK | | |
| | QLGRNVKAYVDDIVVKIHKAFDHANNLQETFD | | |
| | SLRAAGIKLNPEKCVFSVRAGKLLGFLVSERGIE | | |
| | ANPEKIDAIQQMKPPSSVHEVQKLAGRIAALSRF | | |
| | LSKAAERGLPFFKTLRGAGKFNWTPECQAAFD | | |
| | KLKQYLQSPPVLISPPLGSELLLYLAASPVAVSA | | |
| | ALVQETESGQKPVYFVSEALQGAKTRYIEMEKL | | |
| | AYALVMASRKLKHYFQAHKVIVPSQYPLGEILR | | |
| | GKEVTGRLSKWAAELSPFDLHFVARSAIKSQVL | | |
| | ADTTEYEAILLGLRKAKALGVRRLLIRTDSKLV | | |
| | AGHVDQSFEAKEEGMKRYLEAVRSMEKCFTGI | | |
| | MVEHLPRDQNEEADALAKSAACGGPHSPGIFFE | | |
| | VLHTPSVPMDSSEVMVIDQEKLGEDPYDWRTPF | | |
| | VKHLETGWLPVDEAEAKRLQLRATKYKMVSG | | |
| | QLYRSGVLQPLLRCISFAKGEEMAKEIHQGLCG | | |
| | AHQAARTVASKGLDIIGPFPVARNGYKFAIVAV | | |
| | EYFSRWIEAEPLGAITSAAVQKFVWKNIVCRFG | | |
| | VPKEFITDNGKQFDSDKFREMCEGLNLEIRFVSV | | |
| | AHPQSNGAAERANGKILEALKKRLEGAAKGKW | | |
| | PEELLSVLWALRTTPTRPTKFSPFMLLYGDEAM | | |
| | TPAELGANSPRVMFSEGEEGREESLELLEGVRV | | |
| | EALEHMHKYTTSTSATYNKKV | | |
| 8087 | KEQFGLRPKDAGNLYRQPYPEWFERVPLPNRFK | ABA93011 | Oryza sativa |
| | VPDFSKFSGQDSTSTYEHISQFLAQCGEASAVD | | Japonica Group |
| | ALRGMIAPIMEKFSSEDFESLSHLTQKVTLHEQ | | |
| | WFAEARRNSRKVNHVCPYLCGSDDEDDDSEIA | | |
| | VAEWVRSKKVVPCQWVKNSGKEERYEFDITKA | | |
| | DKIFDLLLREKQIQLLAGHTIPSVEELGKKRYCK | | |
| | WHNSGSHTTNDCKVFRQQIQAAIEGGKIKFDDS | | |
| | KRPMKVDGNPFPVNMVHTTGRIADGVRTRGFQ | | |
| | VNSAKIINKYQRKYDKQQEKHYEEDDDGFDPH | | |
| | WGVSLLRIRKKVSKIEKPERFQEVEQEINYRLKR | | |
| | TKPKQEWRVKKQAPVADEAAVDAAKRLAKGK | | |
| | SVVIASVNMVFTLLAEFGVKQADVDEVEEESA | | |
| | KLFLSPEQAVFEKPEGTENRHLKPLYINGYVNG | | |
| | KPMSKMMVDGGAAVNLMPYATFRKLGRNTED | | |
| | LIKTNMVLKDFSGNPSDIKGVLNVELTLGNKTIP | | |
| | TSFFVIDGKGSYSFLLGRDWIHANCCIPSTMHQC | | |
| | LIQWQADKIEIVPADRSVNDCLSGKFWDGDFLK | | |
| | VFDFDIQPVEDGEPKLLFWGRRVYTKDTIDDLD | | |
| | DKQRQGFMSADDLEEIDIGPGDRPKPTFISKNLS | | |
| | AEFRTKLIELLKEFRDCFAWEYYEMPGLSRSIVE | | |
| | HRLPIKPGVRPHQQPPRRRKADMLEPVKAEIKR | | |
| | LYDAGYNQIFMAEEDIHKTAFRCPGAIGLFEWV | | |
| | VMTFGLKSAGAMYQRAMNYIYHDSIGWLVEV | | |
| | YIDDVVVKSKEIGDHIANLRKFLRFLVHERGIEV | | |
| | TQRSVNAIKKIQPPENKTKLQEMIGKINFVRRFIS | | |
| | NLLGRLRHYLLSNECTVICKADVVKYMLSAPIL | | |
| | KGRVGKWIFSLTESDLRYESPKAIKGQAVADFI | | |
| | VEHHDDSIGSVEIVLWTFFFDGSVCTHGYGIGL | | |
| | VIISPRGACFEFAYTIKPYATNNQAEYEADLKGL | | |
| | QLLNQLAGEYECKNDTLMIYNEKCQELLKEFRL | | |
| | VTLRHVSREQNTEANDLAQGASGYKPMIKNVE | | |
| | VEVATITADDWRYDVHQYLQDPSQSASRKLRY | | |
| | KALKYTLFDDELYYRMVDGVLLKCLSADQAK | | |
| | VAIGEVHEGICGTHQSAHKMKWLLRRAGYFWP | | |
| | TMLEDCFRYYKGCQYCQKFGAIQRAPVSAMNP | | |
| | IIKPWPFRGWGIDMIGIINPPSSKGHKFMLVATD | | |
| | YFTKWVEAIPLKKADSGDAIQFVQEHIIYRFGIP | | |
| | QTMTTDQGSIFVSDEFVQFADSMGIKLLNSSPYL | | |
| | CTS | | |
| 8088 | DYLEQENRVLREEMTAMQTRMDEMAELIKTM | ABE77575 | Medicago |
| | AEAQTQAQAQIQAQAQALAQAQTQAQTLTEAQ | | truncatula |
| | ARSQAPPPPPPVRTQAEASSSWTLCADTPTQSAP | | |
| | QRSTPWFPPFTAGEIFRPITCEAQMPTHQYTAQT | | |
| | PLPAMRVTPATMTYSAPVIHTIPQTEEPIFHSGN | | |
| | AEAYEEVSDLRAKYDELRRDMKALHEKGKFG | | |
| | KTAYDLCLVPSVQVPHKFKIPDFEKYKGSSCPE | | |
| | EHLKMYVRRMPAYAQDDQILIYYFQESLTGPAS | | |
| | KWYTNLDKTRVQTFRDLCEAFVEQYSYNVDM | | |
| | TPDRSDLQAMTQGDKETFKEYAQRWRDTAAQ | | |
| | VSPRIEEKEMTKLFLKTLNHFYYKKMVGSTPKS | | |
| | FAEMVGMGVQLEEGVREGRLVKNTTPASGTKK | | |
| | TGNHFPRKKEQEVGMVTHGGPQQTYPAYQHIA | | |
| | AITPTSHPFQQTNNHPQIPQYPQMPQYPQIPQYP | | |
| | QFPQNPSPQNTQQQNIQQQNFQQQPYQQYPYQ | | |
| | QYPQQYFQQQPYQQRPQQPRPPRMPINPIPVTY | | |
| | AELLPGLLKKNLVQNRTAPPIPEKLPSWYRLDQ | | |
| | TCDFHEGGRCHNIETCYAFKSAVQRLINDGKITF | | |
| | TDSAPNVQTNPLPNHGAATVNMIENCQKTRPIL | | |
| | NVQHIRTPLVPLHAKLCKVDLFEHDHDLCEICL | | |
| | MNSGGCQKVRNDIQGLLDRGELVVERKSDDVC | | |
| | VITPEGPLEVFYDSRKSTITPLVICLPGPLPYASE | | |
| | KAIPYKYNATMIEEGREVPIPPLSSVDNIVEDSR | | |
| | VLRNGRVVPIVFPKRIDATTNKELRTKDADVAK | | |
| | EVDQPKEAGTSAEFDEILKLIKKSEYKVVDQLM | | |
| | QTPSKISIMSLLLNSEAHKDALMKVLEQAFVDY | | |
| | DVTVGQFGGIVGNITACNNLSFSDEELPAEGRN | | |
| | HNRALHISVNCKTDALSNVLVDTGSSLNVLSKT | | |
| | TYTQLAYQGAPLRRSGVMVKAFDGSRKDVLGE | | |
| | VILPITVGPQVFQVNFQVMDIQASYSCLLGRPWI | | |
| | HEAGAVTSTLHQKLKFVKNGKLVTVNGEEALL | | |
| | VSHLSSFSFIGADDVEGTPFQGFTIEDKNAKRNE | | |
| | ASISSLRDAQKVIQAGGSTSWGKLIELPENKHRE | | |
| | GLGFFPSTGLSTAKKGTFHSSGFIHAIIEDDPESV | | |
| | PRGFITPGVSSHNWVAVDVPFVAHLSKLEIDEP | | |
| | VEQHNPMISPNFEFPVYKAEEEENEEIPDEISRLL | | |
| | EQERKTIQPYGDELEVINLGTKEDKKEIKVGASL | | |
| | ETSVKKQVIELLKEYVDVFAWSYRDMPGLDTDI | | |
| | VVHHLPLKPECPPVKQKLRRTRPDMALKIKEEV | | |
| | QKQIDAGFLVTSNYPQWLANIVPVPKKDGKVR | | |
| | MCVDYRDLNKASPKDDFPLPHIDVLVDSTAKS | | |
| | KVFSFMDGFFGYNQIKMAPEDREKTSFITPWGT | | |
| | FCYKVMPFGLINAGATYQRGMTTLFHDMIHKEI | | |
| | EVYVDDMIVKSITEEDHVKYLQKMFQRLRKYK | | |
| | LRLNPNKCTFGVRSGKLLGFIVSQKGIEVDPDK | | |
| | VKAIREMPAPRTEKEVRGFLGRLNYISRFISHMT | | |
| | ATCGPIFKLLRKEQGIVWTEDCQKAFDNIKKYL | | |
| | LEPPILIPPIEGRPLIMYLTVLENSMGCVLGQQDE | | |
| | TGRKEHAIYYLSKKFTECESRYSILEKTCCALA | | |
| | WAAKRLRHYMINHTTWLVSKMDPIKYIFEKPA | | |
| | LTGRIARWQMLLSEYDIEYRSQKAIKGSILADHL | | |
| | AHQPLEDYRPIKFDFPDEEIMYLKMKDCDEPLF | | |
| | GEGPDPDSVWGLIFDGAVNVYGNGIGAVLLTP | | |
| | KGTHIPFTARLRFDCTNNIAEYEACIMGIEEAIDL | | |
| | RIKNIEIYGDSALVINQIKGKWETLHAGLIPYRD | | |
| | YARRLLTFFNKVELHHIPRDENQMADALATLSS | | |
| | MIKVNHHNDVPLISVKFLDRPAYVFAAEAVFDD | | |
| | KPWFHDIKVFLQTREYPPGASNKDKKTLRRLSS | | |
| | NFFLNGDILYKRNFDTVLLRCVDKYEADLLIHEI | | |
| | HEGSFGIHPNGHTMAKKILRAGYYWMTMESDC | | |
| | YKHTRKCHKCQIYADKIHMPPTTLNLLSSPWPF | | |
| | SMWGIDMIGRIEPKASNGHRFILVAIDYFTKWV | | |
| | EAASYANVTKQVVVKFIKNHIIYRYGIPNRIITD | | |
| | NGTNLNNKMMKELCDDFKIEHHNSSPYRPQMN | | |
| | GAVEAANKNIKRIVQKMVVTYKDWHEMLPFA | | |
| | LHGYRTSVRTSTGATPFSLVYGMEAVLPVEVEI | | |
| | PSLRVLMEADLSEAEWVQNRYDQLNLIEEKRM | | |
| | TALCHGQLYQKRMKQAFDKKVRPREFKEGDL | | |
| | VLKKIFSFQPDSRGKWAPNYEGPYVVKRAFSGG | | |
| | AMTLQTMDGEELPRPVNIDAVKKYFV | | |
| 8089 | TKQSSSKTEVEPTNVIPITLDDFEGEDCKSMKEY | AAM19047 | Oryza sativa |
| | IKEITQEALMRACTRTRQGMIIKPGPRPKLTLDL | | Japonica Group |
| | VSNEEVTQSIQQQVASTIDSSMIIFKNKLDATIEG | | |
| | RFDEFLRTKFGPLMADFMLKDKASTSASQAPID | | |
| | QTSRRTDGAAQTAGPTGPDGRSDRILPRRLDRD | | |
| | SGRSDRASGRRSDRALDRTFDSPVSTVATNSQV | | |
| | PPHVPNAYNDVARGYPPDTRQGQYNHITPQTQP | | |
| | IRPPNPPPNQHRPDNMEEIISGIIRDKFGIEARNR | | |
| | AKVYQKPYPDYYDNVPFPRGYRVPEFTKFSGE | | |
| | DSRTTWEHRFRDVRNRCYSLNITDRDLAGLTEN | | |
| | GLIAPLRERLDGQQFLDVSQLMQKALAQESRV | | |
| | KDNKKFVRPYEKKPNVNLIDYPEASDSEGEGDH | | |
| | DMYVAEWSWTNKNKPFVCSNLMPTPRKDWQS | | |
| | EVQSAIDEGRLKFTDSSKMKLDHDPFPVNTINF | | |
| | NDKKMLIRPEQAESTKGKGVVIGEPRPKMIVPK | | |
| | KPENRANEEKREGKRITVEARTSEVITIKVGSHD | | |
| | VPIPSGDEVGESSSNKPKAGTSSSQSAGLTRPSG | | |
| | RSDRSHTAGLTGPSGQSDRRPNDGPTDAPGRSD | | |
| | SRHHVGPTGAPGRSDRWSKSGLTGLQHRFDGR | | |
| | FTKGSAGTSSSSSRPNRGHYLPPGTEPKPRRFNE | | |
| | LRPPPVWRRKSVEKEEPIVVEKKEKQSVDKDES | | |
| | SLKEDMDINMVCMLPMEFCAVDEAEVAQFSLG | | |
| | PKDAVFEKPDESNRHMKPLYLKGHIDGKPVSR | | |
| | MLVDGGAAVNLMLYSLFKKMGRGVDELKKTN | | |
| | MILNGFNDEPTEAKGIFSVELTVGNKTLPTAFFI | | |
| | VDVQDFDKVEKLGQGFTSADPLKEVDIGNGTK | | |
| | PRPTFVNKNMRADYKVKIIKLLKEYVDCFAWK | | |
| | YHEMPGLSRELVEHRLPIKPGFRPYKQPPRHFNP | | |
| | LLYDRVKEEIDRLLKAGFIRPCRYAEWVSSIVSV | | |
| | EKKGSGKIRVCIDFRDLNKATPKDEYPMPIADM | | |
| | MINDASGHKNAGATYQRAMNLIFHDLLGIILEI | | |
| | YIDDIVVKSDGMEGHIADLRLAFERMRQYGLK | | |
| | MNPLKCAFGVSAGRFLGFMVHERGVEIYPKKIE | | |
| | KIRDFKAPICKKEVQKLLGMVNYLRRFIFNLAG | | |
| | KIDAFVPILHLKKEADFTWGAKQQEAFEELKRY | | |
| | LSTPPVVRAPKAGKPIRLYIASEDKVIGAVLTQE | | |
| | EDGKVYIITYLSRHLLDAETRPILSGRIGKWAYA | | |
| | LIKYDLAYEPLKSMRGQIACDFIVDHHVDIAYE | | |
| | EDVCLVEVIPWKIYFDGSSCKEGQGIGVVLFSPN | | |
| | GMCYEASVRLEYYCTNNQAEYNALLFDLQVM | | |
| | EMVGAKYVEAFGDSELVVQQVAEVYKCLDGS | | |
| | LNRYLDSCLDIIANFDNFAIRHIARHDNSRANDL | | |
| | AQQASGYDVKKGLFLLFEEPMLDFKFLCEIGEI | | |
| | GDQGRSDRRCAAGLTGDQVQSDRPHAADLTG | | |
| | DPGRSDRPCMAGLTGSGQGAGGHATINLEAKLI | | |
| | VNSDICAQETEEDWRIPLIRYLKDPTLKVDWKIR | | |
| | RQAFKYTLLDEDLYRQNIDDVLLKCLDEDQSK | | |
| | VAMGEGHRFVLVAMDYFTKWAEVVPLKNMT | | |
| | HTEIIDFILKHIIHRFGIPQTLTMDQAKSSNKTLL | | |
| | KLIKKKIEEHPKRWHEVLSEALWAHRISKHGAT | | |
| | KVTPFELVYGQEAILPVEVNLGSLRYIKQDDLS | | |
| | GEDYKTLMGDNLDEVIDKRLKALEEIENEKKR | | |
| | VAKAYNKRVKVKLFQVGDLVWKTILSLGTRFR | | |
| | EFDSRYSSKRQRGMCLHPNLASKVKIGKVAKI | | |
| | WKSTGFLRVGRSDRRVLGGQTAG | | |
| 8090 | QHYHDTIQQENQLIPSFFTYIAKLKQDLDSLPDE | XP_0012490 | Coccidioides |
| | FFHKRRSKVKDYPQTKKTPVKPLPKISEEEHKQ | 02 | immitis RS |
| | QIDEELCLRCGQPGHKTKFCTNSSNKSQQTDKK | | |
| | NKNQAKTRTAKPMQDPGQTLERQGVNPIKKAS | | |
| | RCKQAALLDSGTTVNSISYKLASQLDWDQPETP | | |
| | MEVIEMLNRAEADWYSIYKTQLTITDSMGTIKM | | |
| | KKYYCPSRQRFYKNDILIFSASEKEHEKHVRLV | | |
| | MEYLREYQLFAKLAKCAFKRQTISYLGYIIDNE | | |
| | GIKMDPKQIQVITEWLLLQSFHNIQIFLGFANFY | | |
| | QRFIQKYSVIVALLTDLLKSSEKRRKKEPFLLTP | | |
| | TTRKVFCELQAVFFREPVIQHYNPECRIHLETDT | | |
| | SEHAAEMVQGRPGGTKIIDVLNLLLEAQGDDSS | | |
| | VQARFTKTESQQSE | | |
| 8091 | NSSDSTNNTSPHSSATCSTAPTTSSVPAVTFLPTP | XP_502848 | Yarrowia |
| | QSPDFSHEHARYLNWLRSIYPVHTIPIFTGDAVL | | lipolytica |
| | VSQNAQLAHDWLIAVENFLCTFPVPHHARPHLL | | CLIB122 |
| | GSRLFGSAGLWWRQSMAKNILSNWHEFKSNFA | | |
| | SYWCPEFNSQTESHFFHKVRQGVAETAAEFAQ | | |
| | RRLQVGKMAGVPKTYHVSLRRSRKLIYDPDNQ | | |
| | VYLCMVKPRRDSEAVSREELELISKNKDIVVNT | | |
| | PPDLAKSPFCVNYGLCRHETEFVEYHVNRMLEE | | |
| | GLVDPTQSVYGAPVLIIISKDGEFRMCTDHRILD | | |
| | DRSINDRFWLPKTDEILSQIGHNGVFSKLHLFSG | | |
| | YYQAFKKPTGLSNNVISIFPLDLREDRDNHLGLS | | |
| | EVLDQLGGCLSGVAFDEFDNLIVCSKDRETHTA | | |
| | DLDNVLRVLRQRHIYVNKYQSDMFKSSLELLG | | |
| | HIVDKQTCRPDPLKVCTIVNWRAPLNTTDTASF | | |
| | LHLAGYYRRYIPDFALVARPMALLCGGNKPFD | | |
| | WTEKCQSAFDLIKTTLVRAAPVRLETLRGAYRL | | |
| | STEIFETCFSVVLEQQDASGMFHVVDRKSARFH | | |
| | GLELRFTEYEKNVKAIVYALRKWRIGHGLFLIQ | | |
| | TRFPLSRDIILNPAYLKGSSLSRWLHFIHAHQFE | | |
| | VADISQNKMQKNGTDGLKRGVEVDGKMGVDN | | |
| | VVDSGANSLAKRVCVEN | | |
| 8089 | TKQSSSKTEVEPTNVIPITLDDFEGEDCKSMKEY | AAM19047 | Oryza sativa |
| | IKEITQEALMRACTRTRQGMIIKPGPRPKLTLDL | | Japonica Group |
| | VSNEEVTQSIQQQVASTIDSSMIIFKNKLDATIEG | | |
| | RFDEFLRTKFGPLMADFMLKDKASTSASQAPID | | |
| | QTSRRTDGAAQTAGPTGPDGRSDRILPRRLDRD | | |
| | SGRSDRASGRRSDRALDRTFDSPVSTVATNSQV | | |
| | PPHVPNAYNDVARGYPPDTRQGQYNHITPQTQP | | |
| | IRPPNPPPNQHRPDNMEEIISGIIRDKFGIEARNR | | |
| | AKVYQKPYPDYYDNVPFPRGYRVPEFTKFSGE | | |
| | DSRTTWEHRFRDVRNRCYSLNITDRDLAGLTEN | | |
| | GLIAPLRERLDGQQFLDVSQLMQKALAQESRV | | |
| | KDNKKFVRPYEKKPNVNLIDYPEASDSEGEGDH | | |
| | DMYVAEWSWTNKNKPFVCSNLMPTPRKDWQS | | |
| | EVQSAIDEGRLKFTDSSKMKLDHDPFPVNTINF | | |
| | NDKKMLIRPEQAESTKGKGVVIGEPRPKMIVPK | | |
| | KPENRANEEKREGKRITVEARTSEVITIKVGSHD | | |
| | VPIPSGDEVGESSSNKPKAGTSSSQSAGLTRPSG | | |
| | RSDRSHTAGLTGPSGQSDRRPNDGPTDAPGRSD | | |
| | SRHHVGPTGAPGRSDRWSKSGLTGLQHRFDGR | | |
| | FTKGSAGTSSSSSRPNRGHYLPPGTEPKPRRFNE | | |
| | LRPPPVWRRKSVEKEEPIVVEKKEKQSVDKDES | | |
| | SLKEDMDINMVCMLPMEFCAVDEAEVAQFSLG | | |
| | PKDAVFEKPDESNRHMKPLYLKGHIDGKPVSR | | |
| | MLVDGGAAVNLMLYSLFKKMGRGVDELKKTN | | |
| | MILNGFNDEPTEAKGIFSVELTVGNKTLPTAFFI | | |
| | VDVQDFDKVEKLGQGFTSADPLKEVDIGNGTK | | |
| | PRPTFVNKNMRADYKVKIIKLLKEYVDCFAWK | | |
| | YHEMPGLSRELVEHRLPIKPGFRPYKQPPRHFNP | | |
| | LLYDRVKEEIDRLLKAGFIRPCRYAEWVSSIVSV | | |
| | EKKGSGKIRVCIDFRDLNKATPKDEYPMPIADM | | |
| | MINDASGHKNAGATYQRAMNLIFHDLLGIILEI | | |
| | YIDDIVVKSDGMEGHIADLRLAFERMRQYGLK | | |
| | MNPLKCAFGVSAGRFLGFMVHERGVEIYPKKIE | | |
| | KIRDFKAPICKKEVQKLLGMVNYLRRFIFNLAG | | |
| | KIDAFVPILHLKKEADFTWGAKQQEAFEELKRY | | |
| | LSTPPVVRAPKAGKPIRLYIASEDKVIGAVLTQE | | |
| | EDGKVYIITYLSRHLLDAETRPILSGRIGKWAYA | | |
| | LIKYDLAYEPLKSMRGQIACDFIVDHHVDIAYE | | |
| | EDVCLVEVIPWKIYFDGSSCKEGQGIGVVLFSPN | | |
| | GMCYEASVRLEYYCTNNQAEYNALLFDLQVM | | |
| | EMVGAKYVEAFGDSELVVQQVAEVYKCLDGS | | |
| | LNRYLDSCLDIIANFDNFAIRHIARHDNSRANDL | | |
| | AQQASGYDVKKGLFLLFEEPMLDFKFLCEIGEI | | |
| | GDQGRSDRRCAAGLTGDQVQSDRPHAADLTG | | |
| | DPGRSDRPCMAGLTGSGQGAGGHATINLEAKLI | | |
| | VNSDICAQETEEDWRIPLIRYLKDPTLKVDWKIR | | |
| | RQAFKYTLLDEDLYRQNIDDVLLKCLDEDQSK | | |
| | VAMGEGHRFVLVAMDYFTKWAEVVPLKNMT | | |
| | HTEIIDFILKHIIHRFGIPQTLTMDQAKSSNKTLL | | |
| | KLIKKKIEEHPKRWHEVLSEALWAHRISKHGAT | | |
| | KVTPFELVYGQEAILPVEVNLGSLRYIKQDDLS | | |
| | GEDYKTLMGDNLDEVIDKRLKALEEIENEKKR | | |
| | VAKAYNKRVKVKLFQVGDLVWKTILSLGTRFR | | |
| | EFDSRYSSKRQRGMCLHPNLASKVKIGKVAKI | | |
| | WKSTGFLRVGRSDRRVLGGQTAG | | |
| 8092 | HLPSATLDANPRMFKPRLYRVSPCDRQAIDLVF | CAJ41904 | Ustilago hordei |
| | DELTWQGHLTTAPPGTPCSWPVFVVYHEGKPC | | |
| | PVVDLRQLNDVVDPDVYPLPTPDKLREKLAGA | | |
| | KYITMFDLCKAFYQMLLHPDDHWKATVLTHC | | |
| | GQETLSCTIMGQSHSVSFLQQVLTEAFKVDRLS | | |
| | TMAFVYVDDFGVHSNSLDEHMDHIHTVLGVIQ | | |
| | TLGLTLAQDKAHVACKEVPLLGHLVSRQGTRT | | |
| | MPSKCEAIKSIPYPAMLNQLEHMVGFFSYYKNY | | |
| | VPHFSALIAPLQHLKTTLLRLSPKTRQARKCYCT | | |
| | GMSVPDDTSTRQSLTKLKTILQDWALQFPDYSQ | | |
| | PFLLYVDTSQQHGFALALHQNHQDSGIGDSIQCI | | |
| | DAIHLNSTDASAKAPVWFDSHALKPAEKSYWP | | |
| | TELEAAAAVWALFRMKRFLDALPGPHLLFTDH | | |
| | LAVTSIADAKPFSSTPAARNPRLVRFTLILAEFCP | | |
| | KLWILHRKGIYMAHVDALSRIQASETELASFHA | | |
| | HELIIDPGLIAHILQSQQSDSMLQLLHQELTATG | | |
| | KGTLPFDNGSFGLNADNILCRITPSGVWKPCIGT | | |
| | AALPRIIGLVHTGHLGTKATFDRFRAVAYAPHL | | |
| | LCHVEDFVKCCTQCQQMRTLHHWPYGSLQPLP | | |
| | APDTPFTTISCDFIVCLPLACTLFDLDPVDTVLIL | | |
| | TDTATCRIYLLSSTTTWSTERWSLRYVEQLLPH | | |
| | VGWPKKIISDRDLQLTSQFWCSLNTCYSCELIFS | | |
| | TAHHKSNGQSKRAIQSVELLLRGLCNAWSDDW | | |
| | ADHLPLVELLLGNRPNASMNAAPNDLLYGLRL | | |
| | HDPFTMLQLVMSLSDLTLLDCCLALCQQALDH | | |
| | LALVQAYMHQWYNSLHTPPPKLAISDWVWLEL | | |
| | HDGYLLPPSFLPPDQRLGIQHIGPYPIKHMVSNL | | |
| | AYEISLPLESHLHPVISIQHLEPYMPSDKPITTSLV | | |
| | TEILKEHKTHCCSKQYLVCFEHASCDKWVLENT | | |
| | VTNPAILEQWHLRRPLLPPSASPD | | |
| 8093 | TGDFYKRAFWKRTILNPGLRHRSSRLNYRVTHL | EAQ91761 | Chaetomium |
| | DEACGDRWAETPDDVDRNYQWQWDGYPYPQ | | globosum |
| | LLPVAQNGWLKIREPATPLRQTVPHLARIRRAIS | | CBS148.51 |
| | PRIGSCQPLRHTRHLQHHQQSDDPMPTPAVDRQ | | |
| | LRARQLLRFVGTDHPHVTHVDRKDIDHARSFYS | | |
| | WFKKWRPVTTTSTPSISSVGSNNHHQAVTTDCP | | |
| | SERNNQPVETASNASEEISTPTLYTHASSTRPSSP | | |
| | TPTTSDLPSTGNTPKTSTYRRSTSTDNNVDMAD | | |
| | NGRRQPVPDPDGPLTAQAAAALMAAAFRQHRE | | |
| | NQNADMGNLLAAAIDHQQRQQPAASTALQAV | | |
| | DVGYFDPSAKDPSGAGLISGGKINKYTDIFPFCD | | |
| | RLVDLAATHGDDAVRRIWSQCLQGPALVWHS | | |
| | HILTDDDRELLRTATINAICNKLKSRFKIDYSVA | | |
| | LDTLKQSRFTMSDVANDKDIMAFVQTMMRNA | | |
| | KACDMSRHGQLIAAFEALDGDIQSELDKPTSTT | | |
| | EIDSFLRQIQERESVLRKRAQRFRQPQQPYRQHH | | |
| | IGIRGNNNNSHSGTVINNNGINHKAKIRGQGLQP | | |
| | QGQQWQYNQYNVGNQPANNQRQYGQQQQQA | | |
| | QQPGAVVPENRRLPAPPQRNQYRPPTPGRAIPA | | |
| | FHGSAQYQQPSVEDAPEQDVAGPDDFGPAESY | | |
| | YGNAPYPDDEYPAAPEWLDVDHRGPDTDTTDT | | |
| | PDDVVAQFVSLSIASKCRHCAKSFPSNNKLMAH | | |
| | IYADHLKRPRPDRKARADTAGAIPSAREVVDAH | | |
| | LAEAHPVADDPMHLSKIVESKNHPRPRSRLYRN | | |
| | VGRQAMVGRPHTVIRHRASPLMVRGIGKGVHN | | |
| | TLDYAIIDLNFYGTLPSGSTAIASFTREVTIVDDL | | |
| | RANMLLGMDCMTPEKFDILLSDEAIVINSCGGIR | | |
| | IPITTKRHGKPIKSKVIAKHRISIPPQTLASIAVNH | | |
| | AVHVDDGQTLLFEPATLNVSVFAAVADCHMES | | |
| | AIVRNDTNRPITVHANQCVGHLVSMEPDCQAY | | |
| | LVDDAAAAELAVHKPAPPPEERRLATMTEDDIK | | |
| | LNTIQHPCGVTIYNLPPAQMAPLWSLVTEYQDV | | |
| | FKDKGFVNLDQDQWMRVKLRPGWHETLPKKC | | |
| | RIYPMNAEDRGVVKDTIGKLESGGKATKTRFQ | | |
| | VPFSFPVFVVWRTMPDGTRKGRMVVDIRMLNK | | |
| | IVLPDAYPMKSQDDIMARLANAKYITILDAVAF | | |
| | YYQWLVDPRDRWVFTMNTPEGQYTFNCVVMG | | |
| | YRNSNAYVQRQMNLLLKHIDAADAYCDDVAIG | | |
| | SRKFDTDDGHLAHLRRVFDALRRRNISIGPSKSF | | |
| | IAFPSATVLGRMVNSMGMSTTTERLSAITKLNFP | | |
| | ATLKDLEYFIGATGWLRHNVPLYSILVEPLQRR | | |
| | KTALLKTRNRKGKRRSWSHAVQLLLPTSEELAS | | |
| | FEAVKAALSRHTTLAFFRDADPFCIDVDVSALGI | | |
| | GAEVYHIEPAALQKVTKDGLIVKYPPRTAIQPL | | |
| | AYLSRTLSLAERDYWPTEMEVLGLVWVLAKCK | | |
| | RWITATKSSPLYVFTDHKSILGLNNRTADITSST | | |
| | STTNKRLIRAAEFFSTFDLRIQHKPGKFHVVADA | | |
| | LSRLPSTNNVTDPQAPGGLDNLPNDREEHWAFC | | |
| | AATAPLRLPSIRTFPEPADKDVDLGQPTATTTSG | | |
| | IVTLDIHPEFVERLQEGYLQDPVWKRTMTVIRQ | | |
| | NNSLLPANRANLPFELHKGLLWKTGGQVPRLC | | |
| | VPRTCLTDILNAIHDGNHRGFQALRTRLSNFCIS | | |
| | QPTKMLRAYVNACPQCKANDRRHHSPYGSLQP | | |
| | LTGNECPYYMITIDFIVDLPTSTDKLDVALVIVD | | |
| | KLSKETQIVLGKSTWKSSHWGPQLLNRLLTAN | | |
| | WGLPKVILSDRDPKFTAALWRAIWKTLGTNLL | | |
| | YTTAYHPSTDGQSERTIQTIESALRHYIQALDDF | | |
| | TRWPETVPRLQFEHNNIRSRTTGKSPNEIVKGFN | | |
| | PVAVADVIGDHQPARDPNLPQLRMEAHDAVAI | | |
| | AAMTMKHYYDRRHMPRFFDVGSKVWLRVHK | | |
| | GYNMPATDLIGPKFSQQYAGPLEVVERVGRSA | | |
| | YRLRLPPSWRIHDVVSIDHLEPHTFDPYGRQLPA | | |
| | IQPVTTHNQTVKAIVSHRLRGNGNQYLVKYDG | | |
| | LGAEFDQWLPEQRLASIAPGILQQWLQQQQHH | | |
| | K | | |
| 8094 | PAITQYDPALPLFQPIGRTNNNITINPIACPVYRK | EAU82224.2 | Coprinopsis |
| | LSHDDFLLAVDANGTETACIRQETAVSLWTTLK | | cinerea okayama |
| | DIGLAINKAGNPGRNEAVHLLNQYYEIPHYKFD | | 7#130 |
| | RLGLQRPLILDLVRPNLHNIFNPLYAWGPKEYL | | |
| | TKTYQALQIVGRVALRWIDDAKKVCQELGPDC | | |
| | GWDWEEGNEIEEEAPPMPIQAHPLNPYNDLTTR | | |
| | DPRVRPYPARQEIRNSTNRDLILHPTASSAARAN | | |
| | MQIVVHPNRSNAARANNSVTIYRRGNRMARAS | | |
| | AYSTDGSEYVIRNGKQTNTNF | | |
| 8083 | LHDDLQRGPSIIKNTPPPFVIQFGSLPPVTFFEYG | >BAB08213.2_2 | [Oryza sativa |
| | SKVYMQQAQDVTQFQEAQSKKQRKRASAKAK | | Japonica Group] |
| | KERRTLMLEARTLLKESVVAEIKGDIQAAQKLR | | |
| | VKASNRRSITASLRAPDPVATPKLPTPTVQHTEV | | |
| | ELLEALEAVSDNLRRHISHTRRANSPHPLRNYR | | |
| | RKYRKVQRLHQLVSSRIAQSSLLEEDWSLDTSV | | |
| | LIKKVFKFPSILEPPYDLFPDEWACEPTKIKEKVR | | |
| | CAIMKEYWKRRDREHLVLPGSTIFVDYNTYTPR | | |
| | QQSTWESCLHLSAVGGSSDYNNNRFAVLRSEA | | |
| | PAPRSEDLRQELRELQDRMAQLGRRLQDHEAP | | |
| | RSSSTQAGGRSRRYQPSYHPQHDRRTLAPRRTL | | |
| | PSTQVMHQRQTALPPRWNRWSRHQDYPTSSRL | | |
| | AQEWRVREAPSSQVPPHVPSSPRREVYTQRRRE | | |
| | TNAPNPATRQVAPPLLPTPSIPPRRQHAPTENQR | | |
| | KRERRRNNRYALYRELEDLVLKHTQVRVRPDG | | |
| | EVHQEDERIVFRISPSLERDARYNYLIARLTPKP | | |
| | RRTLDVADKNREQALTQPCPVTILQRGKGPVQ | | |
| | ATLGISLSTSARQSKENQSTPMEGVEQTPVEQV | | |
| | DKASRQEEAIINPMVDVLPQQESSSVPPARVEQ | | |
| | VAGSKNIEDPKESIVMCSALAAHYETKPNAAW | | |
| | VPPPVTHDFTYPSDEEIVPNPRANFSKTFLPQLD | | |
| | QVASRPGANTRMKAIAIKNVEATPSQARKDLED | | |
| | HVEVEDLDELESTSSSSLEVNLNLPRYNELNPSL | | |
| | PSDGEGYPNNFDSAPAHVTAEGDPRQHARQHA | | |
| | PRGENPSIGNWATMKEVFKKHFVAMKKDFSIV | | |
| | ELSQVRQWRDEAIDDYVIRFRNSFVCLAREMHL | | |
| | EDAIEMCVHGMQQHWSLEVSRREPKTFSALSS | | |
| | AVAATKLEFEKSPQIMELYKNASAFDPTKRFNA | | |
| | TKPSGSGNKPKVPTEANSTKVFSTAPQGQVPMI | | |
| | GAKNEQVGGRQRSTLQDLLKKQYIFRRELVKD | | |
| | MFNQLMEHRALNLPEPRRPDQVTMTDNPLYCP | | |
| | YHRYIGHAIEDCIAFKEWLQRAVNEKRINLDAD | | |
| | AINPDYHAVNMVSVEPFPQKQREGRRATSWAP | | |
| | LAQVEDQIAKIMLTKAPATHVEASHGDNNRAW | | |
| | SIVRWKPQPMSFPPRRPQMKLSPHTHPTSRRWL | | |
| | DPSRRRPPPRFVPFSEGDESFPRRGRELPTLAQFL | | |
| | PKGWEQSSTSTREAKGVNNSIPTPDIAPCNVILT | | |
| | YNDSTSTGSDETFTGREREIFHAELDPEKTKVEE | | |
| | VNISLRGGKTLPDPHKSKVPNVDKPAKKASPPG | | |
| | EAPEAPETKTGSKEKPAVDYKVLAHLKRIPALL | | |
| | SVYDALMMVPDLREALIKALQAPEVYEVDMA | | |
| | KHRLYDNPLFVNEITFADEDNIIKGGDHNRPLYI | | |
| | EGNIGSAHLRRILIDLGSAVNILPVRSLTRAGFTT | | |
| | KDLEPIDVVICGFDNQGKPTLGAITIKIQMSTFSF | | |
| | KVRFFVIEANTSYSALLGRPWIHKYRVVPSTLH | | |
| | QCLKFLDGNGVQQRITSNFSPYTIQESYHADAK | | |
| | YYFPVEENKQQLGRTTPAADIIVEPGTETTPEHV | | |
| | YPIYYTNIAQSKTLYLNTDHLGGNFSRKRETAQ | | |
| | KQRRCANYHHLTGKTESKQGSRACTTGSGQEE | | |
| | SCRDRGGKSGCSVHAAPSPLHFSFYPAREEACT | | |
| | TMKAEPRMARLLEKAGINLQRNNRLPPPPAVCE | | |
| | DWWAQAEEFIKRRCKEQPKYGLGYINVDEPDD | | |
| | EDEVFEDDIFHCCTISTTTRGDALLQQHPFEVAA | | |
| | VGVEEELDVAGALKQLDDGGQPTIDELVEMNL | | |
| | GTEDDPRPIFVSGMLTEEEREDYRSFLMEFRDCF | | |
| | AWTYKEMPGLDSRVATHKLAIDPQFRPVKQPP | | |
| | RRLRPEFQDQVIAEVDRLINVGFIKEIQYPRWLA | | |
| | NIVPVEKKNGQVRVCVDFRDLNRACPKDDFPLP | | |
| | ITEMVVDSTTGYGALSGYNQIKMDLLDAFDTAF | | |
| | RTPKGNFYYTVMPFGLKNAGATYQRAMQFVL | | |
| | DDLIHHSVECYVDDMVVKTKDHEHHQEDLRIV | | |
| | FERLRRHQLKMNPLKCAFAVQSGVFLGFVIRHR | | |
| | GIEIEPKKIKAILNMPPPQELKDLRKLQGKLAYIR | | |
| | RFISNLSGRIQPFSKLMKKGTPFVWDEECQNGF | | |
| | DSIKRYLLNPPVLAAPVKGRPLILYIATQPASIGA | | |
| | LLAQHNDEGKEVACYYLSRTMVGAEQNYSPIE | | |
| | KLCLALIFALKKLRHYMLAHQIQLIARADPIRYV | | |
| | LSQPVLTGRLGKWALLMMEYDITFVPQKAIKG | | |
| | QALAEFLATHPMPDDSPLIANLPDEEIFTAELQE | | |
| | QWELYFDGASRKDINPDGTPRRRAGAGLVFKT | | |
| | PQGGVIYHSFSLLKEECSNNEAEYEALIFGLLLA | | |
| | LSMEVRSLRAHGDSRLIIRQINNIYEVRKPELVP | | |
| | YYTVARRLMDKFEHIEVIHVPRSKNAPADALAK | | |
| | LAAALVFQGDNPAQIVVEERWLLPAVLELIPEE | | |
| | VNIIITNSAEEEDWRQPFLDYFKHGSLPEDPVER | | |
| | RQLQRRLPSYIYKAGVLYKRSYGQEVLLRCVD | | |
| | RSEANRVLQEVHHGVCGGHQSGPKMYHSIRLV | | |
| | GYYWPGIMADCLKTAKTCHGCQIHDNFKHQPP | | |
| | APLHPTVPSWPFDAWGIDVIGLINPPSSRGHRFIL | | |
| | TATDYFSKWAEAVPLREVKSSDVINFLERHIIYR | | |
| | FGVPHRITSDNAKAFKSQKIYRFMEKYKIKWNY | | |
| | STGYYPQANGMAEAFNKTLGKILKKTVDKHRR | | |
| | DWHDRLYEALWAYRVTVRTPTQATPYSLVYG | | |
| | NEAVLPLEIQLPSLRVAIHDELTKDEQIRLRFQEL | | |
| | DAVEEERLGALQNLELYRQNMVRAYDKLVKQ | | |
| | RVFRKGELVLVLRRPIVVTHKMKGKFEPKWEG | | |
| | PYVIEQAYDGGAYQLIDHQGSQPMPPINGRFLK | | |
| | KYFV | | |
| 8095 | SKEVGATPGLVPTHSPEVQGPKSYANVVSSRPS | AAD08951 | Arabidopsis |
| | LTKFNVDVSVVDGKSMVVVPDVVLEDSVPLW | | thaliana |
| | DDFLVGRFPSSAPHIAKIHVIVNKIWNLGDKSIRI | | |
| | DVFAVNDNTVKFRIRNASARLRALRRGMWNIC | | |
| | DLPMIVSKWTPIVEDAQPEIKSMPMWVVIKNVP | | |
| | YSMFTWPGLVAVGNDLVESVPLVDSQALEVVK | | |
| | GDIAEEVEEGEIASNSNQKSVQGEKIQEEGDWL | | |
| | TVSSSGGKKYISKVRKDFNLWSILEEVQNEDSV | | |
| | GKETEDSVKGVLEVVVVEGKEEMALNKTQQV | | |
| | KSCFDGASTRVSIPRSSKKAHKFVSVPNQKATD | | |
| | VLPRQFGCVLETRVIESKVPVIFAKVFKDWQMV | | |
| | SNYEFNRLGRIWVVWSSSVQLQVIFKSSQMIVC | | |
| | LVRVEHYDVEFICSFIYASNFVEERKKLWQDLH | | |
| | NLQNSVAFRNKPWLLFGDFNETLKMEEHSSYA | | |
| | VSPMVTPGMRDFQIVVRYCSLEDMRTHGPLFT | | |
| | WGNKRNEGLICKKLDRVLLNPEYNSAYPHSYCI | | |
| | MDSGGCSDHLRGRFHLRSAIQKPKGPFKFTNVI | | |
| | AAHPEFMPKVEDFWKNTTELFPSTSTLFRFSKK | | |
| | LKELKPILKDLSRNNLSDLTRRATYAYEELCRC | | |
| | QTKSLTTLNPHDIVDESLAFERWEKERHLLNAI | | |
| | HEVMDPQGTRPPNQDDIKIEAVRFFSDLLSSQPS | | |
| | DFTGISVDELKGILQYRYSLHEQNLLVAEITEAE | | |
| | VMKVFFSIPLNKSPGPDGYTVEFFRETWSVIGQE | | |
| | VTMAIKSFFTYGFLPKGLNSTILALIPKRTYAKE | | |
| | MKDYRPISCCNVLYKAISKLLANRLKCLLPEFIA | | |
| | PNQSAFISDRLLMENLLLASELVKDYHKDGLSP | | |
| | RCAMKIDLSKAFDSVQWPFLLNTLAALDIPEKFI | | |
| | HWINLCISTASFSVQVNGLRQGCSLSPYLFVICM | | |
| | NVLSAMLDKGAVEKRFGYHPRCRNMGLTHLCF | | |
| | ADDIMVFSAGSAHSLEGVLAIFKDFAAFSGLNIS | | |
| | LEKSTLFMASISSETCASILARFPFDSGSLPVRYL | | |
| | GLPLMTKRMTLADCLPLLEKIRSRISSWKNRFLS | | |
| | YAGRLQLLNSVISSLTKFWISAFRLPRACIREIEQ | | |
| | ISAAFLWSGTDLNPHKAKVAWHDVCKPKSEGG | | |
| | LGLRSLVDANKICCFKLIWRLVSAKHSLWVNWI | | |
| | QNNLIRTVAEALSSHRRRSHRDDILNDIEEELEK | | |
| | LLCRGICTEQDRSLCRSIGGQFKAKFFSPEIWHQI | | |
| | REQGLVKQWHKAIWFSGATPKFTFISWLAAHD | | |
| | RLTTGDKMASWNRGISSVCVLCNISAESRDHLF | | |
| | FSCNFSSHIWDRLTRRLLLCRYTTNFPALLLLLS | | |
| | GQDFSGTKRFLLRYVFQATIHTLWRERNKRRH | | |
| | GDLPIPSDHIIKFIDRQTRNRLSTITKQGLHKYAD | | |
| | GLRIWFAARDNLTPNH | | |
| 8096 | NKTLRVIQLNVRKQGAVHESLMNDEETQNTVA | BAE66176 | Aspergillus |
| | LAIQEPQARRIQGRLLTTPMGHHKWTKMVPST | | oryzae RIB40 |
| | WREGRWAVRSMLWINKEVEAEQVPIESPDLTA | | |
| | AVIRLPERLIFMASVYVEGGNASALDDACNHLL | | |
| | DAITKVRRDTGVVVEILIMGDFNRHDQLWGGD | | |
| | DVSLGRQGEADPIIDLMNECALSSLLRRGTKTW | | |
| | HGGGHSGDCESTIDLVLASENLADSVIKCAILGT | | |
| | EHGSDHCAIETVFDAPWSLPKHQGRLLLKNAP | | |
| | WKEINTRIANTLAATPSEGTVQQKTDRLMSAVS | | |
| | EAVHALTPKSKPSSHAKRWWTADLTQLRQIHT | | |
| | YWRNHARSERRAGRKVPYLETMAQGAAKQYH | | |
| | DAIRQQKKKHWNQFLADNDNIWKAERYLKSG | | |
| | EDAAFGKIPQLLRADGTTTTDHKEQAEELLAKF | | |
| | FPPLPDNIDDEGTRPQRAPVEMPAITMEEIERQL | | |
| | MAAKSWKAPGEDGMPAIVWKMTWPTVKYRV | | |
| | LDLFQASLEGGTLPRQWRHAKIIPLKKPNKENY | | |
| | TIAKSWRPISLLATLGKVLESVVAERISHAVETH | | |
| | GLLPTSHFGARKQRSAEQALVLLQEQIYAAWR | | |
| | GRRVLSLISFDVKGAYNGVCKERLLQRMKARGI | | |
| | PEDLLRWVEAFCSERTATIQINGQLSEVHSLPQA | | |
| | GLPQGSPLSPILFLFFNADLVQRQIDSQGGAIAFV | | |
| | DDFTAWVTGPTAQSNREGIEGIIKEALHWERRS | | |
| | GATFEAEKTAIIHFTPKTSKLDREPFTIKGQAVEP | | |
| | KDHVKILGVLMDTSLKYKEHIARAASKGLEAV | | |
| | MELRRLRGLSPSTARQLFTSTVTPVVDYASNVW | | |
| | MHAFKNKATGPINRVQRVGAQAIVGTFLTVAT | | |
| | SVAEAEAHIATAQHRFWRRAVKMWTDLHTLP | | |
| | DTNPLRRNTARIKKFRRFHRSPLYQVADALKNI | | |
| | EMETLETINPFTLAPWEARMQTDGEAMPDPQAI | | |
| | PGGSIQIAISSSARNGFVGFGVAIEKQPPQYRKL | | |
| | KLKTFSVTLGARSEQNPFSAELAAIAHTLNRLV | | |
| | GLKGFRFRLLTSNKATALTIQNPRQQSGQEFVC | | |
| | QMYKLINRLRRKGNHIKILWVPASEDNKLLGLA | | |
| | KEQARAATHEDAIPQAQVSRMKSTTLNLARSQ | | |
| | AATTKALPEDVGRHIKRVDAALPGKHTRQLYD | | |
| | GLSWKEATVLAQLRTGMARLNGYLYRINVAQT | | |
| | DQCACGQARETVEHFLFRCRKWTTQRIALLQC | | |
| | TRTHRGNLSLCLGGKSPSNDQQWVPNLEAVRA | | |
| | SIRFAMTTGRLDAV | | |
| 8097 | THANGQTTNKIYVTCICGKLCKNHWGLKIHLA | XP_684355 | Danio rerio |
| | RMKCLEQESKVQRTGPEPGETQEEPGPEATHRA | | |
| | KSLHVPEPQTPSEVVQQRIKWPPASKRSEWLQF | | |
| | DEDVSNIIQAIAKGDADSRLKTMTTIIFSYALERF | | |
| | GCIEKGKTKPTTPYTMNRRATQIHHLRQELRSL | | |
| | KKLYKKATDEEKQPLAELKNILRKKLMILRRAE | | |
| | WHRRRGRERARKRAAFITNPFGFTKQLLGDKRS | | |
| | GRLECLIEEVNRFIEETVSDPLREQELEPNKALIS | | |
| | PTPPAREFSLRGPSLKEVKEIIKASRSASTPGPSGI | | |
| | PYLVYKRCPGLLLHLWKILKVIWQRGRVAEQW | | |
| | RCAEGVWIPKEENSKNINQFRIISLLSVEGKVFFS | | |
| | IVSRRLTEFLLENNYIDPSVQKGGIPGAPGCLEH | | |
| | TGVVTQLIREAHENRGDLVVLWLDLANAYGSIP | | |
| | HKLVELALHRHHVPSKIKDLILDYYNNFKMRVT | | |
| | SGSETSSWHRIGKGIITGCTISVILFALAMNMVV | | |
| | KSAEVECRGPLTKSGVRQPPIRAYMDDLTITTTT | | |
| | VPGSRWILQGLERLIAWARMSFKPSKSRSMVLK | | |
| | KGKVVDKFHFSISGSVNPTITEQPVKSLGKLFDS | | |
| | SLKDSAAIQKSKKELGAWLAMVDKSGLPGRFK | | |
| | AWIYQHSILPRVLWPLLIYAVPMSTVESLERKIS | | |
| | GFLRKWLGLPRSLTSAALYGTSNTLQLPFSGLT | | |
| | EEFIVVRTREALQYRDSRDGKVSSACIEVRTGR | | |
| | KWNAGKAVEVAESRLQQKALVGTVATGRAGL | | |
| | GYFPKTLVSQVKGKERHHLLQGEVRASVEEER | | |
| | VSRVVGLRQQGAWTRWNTLQCRITWANILHA | | |
| | DFQRVRFLVQAVYDVLPSPSNLHIWGKNETPSC | | |
| | LLCSGRGSLEHLLSSCPKALADGRYRWRHDQV | | |
| | LKAIAASLASAINTSKNHRAPRKAVHFIKAGEKP | | |
| | RALPQLTTGLLHKASDWQLEVDLGKQLRFPHHI | | |
| | AATRLRPDIIAISEASRQLIILELTVPWEERIEEAN | | |
| | ERKRAKYQELVEACRERGWRTYYEPIEIGCRGF | | |
| | AGRSLCKVLSRLGITGVAKKRAIRSASEAAEKA | | |
| | TRWLWIKRADPWTAVGTQVGT | | |
| 8098 | ERSVEEKRKNWRMVDWKEYREKLEANLRKEM | EAU86808 | Coprinopsis |
| | GVGEIEDEDELEVEVDALIRAIGMTTEQVVKILE | | cinerea |
| | RVDWSRGWWNDECRRKKKEFNEARREAWKY | | |
| | RAMPEHPALEEERRIGREYRTLIERTRTECWNE | | |
| | WVREVTELQTWTLNKFIGNTPGDGGLDRMPTL | | |
| | RWTDENGVEVIATDGRSKAKGLVRQLFPERPA | | |
| | ESGVPEGYEYPEPVEYEARMTEERIKGAIKSLK | | |
| | AYKAPGPDGIPNVVWKECVELLAPQLERIFKAV | | |
| | YEKGMYSERWKEWTTVVLKKPGKPRYDTPKA | | |
| | WRPIALMNTMGKILTALLTEDLKYVTEKYSLLP | | |
| | NTHFGGRPGRTTTDAIQLLTSWIKGHWRKGNV | | |
| | VSVLFLDIEGAFPNVVVSRLAHNMRRRRVPEFI | | |
| | VKLIEHQLRDRRTKLKFDDYESEWVPIDNGSGQ | | |
| | GDPKSMLEYLFYNADLIDLVAGLGEELEEGENG | | |
| | EDAPRGSARERGTEKRDENAAAFVDDAWLGG | | |
| | AGATFEEANETLKDMMNRRGGAMEWSKKHNS | | |
| | KFEISKLVYMGFTRRMRRTREGEGGKMTAEER | | |
| | PELEMEGAGNDGGGEGDKVEHGVQENGESEER | | |
| | SGTEGAEDVVQRGDDTKGNIRIGNMVHTSEGD | | |
| | RGEEEEGGISFGDSKTHESTQNLPASNNWSAKD | | |
| | NGHGRLGDPRGDTTINGNAQLDMSKGLDTVVN | | |
| | CCFLAYKSLIALASLVALSSLALPLWFQDTTSDD | | |
| | PESTPTTQGSAISGKETTEETPVADTQAGRSIPRN | | |
| | TDDETGNHRTGCESTKRATTIRN | | |
| 8099 | SSGSRCEDWKRVRNLQRLLLKSYSNVLLAVRR | PZO49854.1 | Phormidesmispri |
| | VTQINAGKNTPGIDKMLVKTGPAKGKLVDLLK | | estleyi |
| | PQNAWQPLAARRVQIPKRNGKRRPLGIPSIIDRC | | |
| | LQAVVKAALEPCWEAQFEPTSYGFRPSRSVHD | | |
| | AIARLYVTANVNNRKKWVLEADIAGCFDTIDH | | |
| | DFLLQQIGHFPARRVIAQWLKAGYVENGIFHPS | | |
| | EAGTPQGGILSPLLANIALHGMETALGITRYAQ | | |
| | GCVKRTVKRVLVRYADDCVVVCDSQVEAEQA | | |
| | QVDLQRFLKFRGLELSEEKTRIVHLSEGFDFLSF | | |
| | NVRHYRSQNTRTGWKLLIKPAKSGGSKRTADG | | |
| 8100 | SSGSSGTPESKQSQPGGRKPPLMCHPAITYDAM | WP_1572103 | Turneriella parva |
| | CSLAGLQRAQISLIEELKRRGEGSKHALSYEDLT | 36 | |
| | ELGALLRSHQYSHRPCRLMTIKVGSKKRDIDSP | | |
| | DWLDRIVQRTYVDTIYPLVQQMACDSSHAYLY | | |
| | KRSIHTALWRLIMNIEHFGYSHVERTDIESFFDSI | | |
| | PHAEMERVIDLHIRDIELNAFSHELLRVAEGFKN | | |
| | SKVGLPTGWLIPPLWANMLLTPVDARLESAGL | | |
| | KFFRYGDDYGILQRSKQEAEFAQGLLESALKPL | | |
| | GLHLKPGYSHKTYTRKLEDGLIVLGHEIRRINNR | | |
| | LTVAISKNSLAETRSGGSKRTADG | | |
| 8101 | SSGSDEVSVTDRSLEQAFNAVFHDRESENDFCT | PCJ98666.1 | Alteromonadaceae |
| | LPLAPEVSEIPLHLRKVYRPSDKLKTYLRFIDKV | | bacterium |
| | VLRHLKYNASVVHSYIKGSSALTAVQAHAKNQ | | |
| | AFFLSDIKSFFPNIGDQDVRKVLMRDSHRIPILDF | | |
| | DQHIERVTKLMTLDGVLPVGFPTSPKLSNGFLH | | |
| | EFDNALAAYCDSTGLTYTRYSDDIIISGMDRAK | | |
| | LTVLREKVQMMLEEHASKSLRLNDEKTRVTHR | | |
| | GNKVKILGLVITPDGQVTIDVSRKHALEGLLHS | | |
| | GGSKRTADG | | |
| 8102 | SSGSLRNFGLPVISSLEDFASSTRLSVSFIKYYLF | WP_2022638 | Enterococcus |
| | QTDSHYKVFSIPKKKGGERIIAQPSRNLKAIQSW | 42 | faecium |
| | ILRNILDRLSSSENSKGFEKGDSILNNALPHSGAS | | |
| | YILSIDIEDFFPSISANKVHSVFRSLGYNSDVCKIL | | |
| | TTFCTYKGRLPQGAPTSPKLANLVSQQLDARIQ | | |
| | GYAGPKGIIYTRYADDLTLSSNTVKKLEKARDII | | |
| | GLISKSEGLKINSLKTKLTGSRSRKSVTGLIVTKE | | |
| | GVGIGRAKYRELRSHIYSGGSKRTADG | | |
| 8103 | SSGSEIPLIYSNRKLYEYIKNNKDDFLQCDIHKES | WP_0575852 | Paeniclostridium |
| | DSILTIPFTYLVRKNENEYRRLSLLHPIAQLQVA | 75 | sordellii |
| | NTLMKYDNLLLNYFNSNSTFSIRTPVGINDSYLN | | |
| | IENRHKLELEWIEKERAKDFSDEENEFVSNYFVI | | |
| | KKFKTITEFYKSDYVKNLELKYKNLIRIDYANCF | | |
| | ENIYTHSLEWAYVGNKNIIKNNLHDERFSAKLD | | |
| | ILAQRINYNETNGLVVGPEISRTLAEVVLARIDK | | |
| | NVYFDLKEKNIIYKRDYEVVRFIDDIMIFYNAEN | | |
| | IGDYIKESIENYSREFKLKINSSKTKYEKRPFFRE | | |
| | HMWISHSKKSIRSFLKYYDGSINYSGYTYDRFIE | | |
| | EFKELICSGGSKRTADG | | |
| 8104 | SSGSKLNKSILESYLQWYPFSKLTENSKCTILSE | WP_0947571 | Staphylococcus |
| | KFFFNFIKNGAIFKEYNTFNFPSHYSQKTSASFR | 11 | aureus |
| | NMTLVSPFVYLYIEVVGYHISKKYTRKSKYVRC | | |
| | YYSGDLSENEFSYKNSYDKFFADINALSSTYDN | | |
| | FYKFDISNFFDAVDINLLFKLINEGEEILDTRSSLI | | |
| | YKRLLQQIGGNKFPTLENSSTLSYLATYIYLDKV | | |
| | DYELEKVLQKNSKIESFQIIRYVDDLYIFFNTME | | |
| | SELNLVSSEIKNVVIDAYRKVKLNLNENKTKLG | | |
| | KSSEVNETLSVALYNHYVYKEEIDIAHFYDKNK | | |
| | ILLFLDDLYSGGSKRTADG | | |
| 8105 | SSGSSRAAGIDGITVDLFTGIAREQIHQLYRQMR | WP_0884289 | Halomicronema |
| | QERYVARPAKGFYLAKQKGGHRLIGIPTVRDRI | 78 | hongdechloris |
| | VQRYLLQSIYPSLENAFSDAVFAYRPGLSIYAAV | | |
| | KRVMERYRYQPTWVIKADIQQFFDQLSWPLLL | | |
| | HQLDQLSLPATWVQWIEQQLKAGIVVSGQFYQ | | |
| | PGQGVLPGSILSGALANLYLNDFDRHCLEADIPL | | |
| | VRYGDDCVAVCQSYLEASRSLALMQDWIEGLS | | |
| | LSFHPEKTTIIPPGQAFVFLGHRFRNGTVEGPAR | | |
| | QKAEGRRSGGSKRTADG | | |
| 8106 | SSGSVWESYKKVRANKGSSGVDGVSLQQFEEK | MBI4970604. | Candidatus |
| | LSDNLYKVWNRLSSGSYFPPAVKEVEIPKKDGG | 1 | Omnitrophica |
| | KRLLGIPTVGDRVAQMVVKDYLEPRLEKEFLN | | |
| | QSYGYLKSDKKISELSGKRRLEIEGEARISRAMC | | |
| | HFRLLECYGQFFDLNSEYGVVIKMSASREIEAIK | | |
| | RSTVKQTYDSILVDLNFGIANAPVVSPHDKFSQT | | |
| | LAKAHKAKVLLYMGEYADAASVALDAMGDA | | |
| | NYKLEDTYQEIFAKGYKAREVLFSPYMVYEEKS | | |
| | NTWTYAGFYCPVAQIETMADNEKADEVDSGGS | | |
| | KRTADG | | |
| 8107 | SSGSATYDNFLLAWQRTVNTTSRMIRDELGMKI | WP_0966735 | Fischerella sp.NI |
| | FAHNLQTNLEYLVQQVKAKDFPYKPLADHKVY | 02 | ES-4106 |
| | VPKPSTTLRTMSLMAVSDVIIYQALVNIIADKAY | | |
| | SYLVTHENQCVLGNIYSGPGKRWMLRPWKKQ | | |
| | YTRFVDCIENLYHAGNPWIASTDIVAFYDTIDH | | |
| | ARLLSLIRKYCGDDQQFQELLQECLAKWAVHN | | |
| | SNITMGRGIPQGSNASDFLANLFLYEIDKEMIVN | | |
| | GYHYIRYVDDVRILASDKSTVQRGLILFDLELK | | |
| | RAGLVAQVTKTSVHEIEDIETEISRLRFIITAPTR | | |
| | NGNCLLVTLPSLPKSEQASGGSKRTADG | | |
| 8108 | SSGSSGLLPLLGKREVWDEFLSYKAEKQHLSRK | MBD891878 | Lachnospiraceae |
| | DARYWTKFVEEEQYRSVTDHILEPDFSLSVPVK | 0.1 | bacterium |
| | LSVNKSNTGKKRVVYSFPEQESMVLKLLGHLLS | | |
| | RYDACLSPACYSFRKNITAKDAVSHILAVPGLS | | |
| | RKYVLKMDIRNYFNSMPVSSLLHVLKEILSDDP | | |
| | FLYSFLERMLTANEAYEHGRLITEERGAMAGTP | | |
| | TSAFFADVYLLSLDNYFAERGIPYFRYSDDILIL | | |
| | ADSPKELLSYREIAAKLIEEKGLSLNPDKLSVTP | | |
| | PGGAFEFLGFSIRGTDCPEKGMPAGKVDLSEAS | | |
| | GGSKRTADG | | |
| 8109 | SSGSRCMQRITKLYNKLLRSNRIFEQDQAGINIS | WP_0272704 | Legionella |
| | DIYTDKKNITKILIRELLNGSYKPMQYDERKVYI | 68 | sainthelensi |
| | NSKMRLIANYSFIDRLLLSILYDLFRERTLNLISP | | |
| | SVYSYISGRSAKQAIQSFCSYLKQIQAPNKQINL | | |
| | YVLRADITNYGGSIPADTHAIFWNYFYDILEEIK | | |
| | DLEQRDCLRIVIEEALRPILHTEDNLPYQKIVGIP | | |
| | VGSPLATLIYNLYLSELDEALSDIPYGFYARYSD | | |
| | DFIYANTDVNQFKEGERRITAILEKLRLRCNPSK | | |
| | NQRFYLTHAGKPSIDSEHFIGSNRIELCGLIIFSD | | |
| | GTRTLKRSIIQKMLERISGGSKRTADG | | |
| 8110 | QYQLQDAYGYCSYPRPQAAKSLLEKSLSDASL | WP_0136598 | Marinomonas |
| | HQACQTMYPRQANFDSSDTDEEHHDAIDELLT | 58 | mediterranea |
| | KLYVSRERIFKREFTPSQLHSVEIEKPEGGTRLLS | | |
| | VPNWHDRTLQKAVTECLGNTLEHIWMKHSYG | | |
| | YRKGHSRLQARDQINQYIQQGYEWVLESDIESF | | |
| | FDSVNWLNLEQRLKLLLPNEPLVPLLMQWVSA | | |
| | AKQTEDEQTLARHNGLPQGAPISPILANLLLDDL | | |
| | DQDMIAKGHQIVRYADDFVLLFKSKAAAESAL | | |
| | DDIITALKEHHLAINLEKTRIVEASQGFRYLGYL | | |
| | FVDGYAIETKREYRKEHAQLDKQLNASSLENEP | | |
| | SLQQEPAVQNEQSTLIGEREKLGTLKL | | |
| 8111 | GWLYNQMAMPETIFQAWYKVASNDGRPGWD | WP_0124658 | Chlorobium |
| | NKSIEDYSLQLEENLKALSQALLTGTYKQGPLM | 87 | limicola |
| | KLVLLKPDGKDRVLLIPGVMDRVAQTAAAIVLS | | |
| | PIIEAELGNCTFAYRPGISREGAAREIDRLHREGY | | |
| | QWVLDADIRSFFDNVRHDLLFQRLVELIDDKEM | | |
| | ISLLHRWLTAEIVDGINPRIQNTMGLPQGCPISPA | | |
| | LANLYLDRFDETMEKEGFKLVRFADDYLVLCK | | |
| | TRPKAEAALKLSETALAELKLELHSDKTRITTFA | | |
| | EGFKYLGYLFIRALVIPTKMHPEEWYDKLGKFK | | |
| | LRKKSEHALPSDPDAMTGETAKFELETDQGEKI | | |
| | ELTKNELLQTEFGCKLLESLDKKQLSVDEFLEK | | |
| | VARQDEERQKEKRDALKKLYSPFLNTKL | | |
| 8112 | SSGSKWKTLKKKRRYITNYQKIDSIKNNADSLF | WP_0897359 | Chryseobacterium |
| | ETIRYYKEKHPNELFIINLNKFVKDIQDSILNTNF | 81 | jejuense |
| | CFTSPKIIPLSKKDQSKCRPIALYNLKDRIIISLTN | | |
| | KYLSEYFDEHFFPESHAFRPKRIYKGKKVVTSH | | |
| | HHAMDSILKYKSDYKGKKLYVSECDISKFYDSV | | |
| | NHTIVKECFKKLISQSNLVIDSNAKRIFYKYLES | | |
| | YSFVHNVKIYNHKKYSDYWQQYKIDNGYFGWI | | |
| | DDDLKDLKYYKSVNHNRVGVPQGGAISGLIAN | | |
| | MVLHFADLELLKKKDSKLHYVRFCDDMVIIHP | | |
| | NKKQCEDYYQVYNESLKKLKLVPHLPLNFNFN | | |
| | NKQILKEFWSEDTKSKSPYRWSGSFRNSTKWIG | | |
| | FVGYEVSFNNEIRVRKRSLKKEKLKQSGGSKRT | | |
| | ADG | | |
| 8113 | SSGSKILQVVDNVERIYREGAGDKATQMIFSDIG | WP_0700435 | Streptococcus |
| | TPKSKEEGFDVYNELKDLLVDRGIPKEQIAFVH | 19 | agalactiae |
| | DANTDEKKNSLSRKVNSGEVRILMASTEKGGT | | |
| | GLNVQSRMKAVHHLDVPWRPSDIVQRNGRLIR | | |
| | QGNMHQEVDIYHYITKGSFDNYLWQTQENKLK | | |
| | YITQIMTSKDPVRSAEDIDEQTMTASDFKALAT | | |
| | GNPYLKLKMELENELTVLENQKRAFNRSKDEY | | |
| | RHTVSYCEKHLPIMEKRLSQYDKDIAQSLATKS | | |
| | QDFVMRFDNQAMNNRAEAGDYLRKLITYNRS | | |
| | DTKEVKTLASFRGFDLKMTTRGPSEPLPETVSL | | |
| | MIVGDNQYTVASGGSKRTADG | | |
| 8114 | SSGSMSKLKRLRSASTKPQLARVLEVDAAFLTR | WP_0658187 | Vibrio cidicii |
| | CLYINKTQNQYHQFSIAKKSGGTRLINAPSKELK | 78 | |
| | SLQKKLSILLLDCIDEINAEKYPRSQLVKPKLRK | | |
| | NGDPDYAAEVLKIKISTAETKQPSLAHGFVKER | | |
| | SILTNAMMHVGKKNVLNIDLNDFFDCFNFGRV | | |
| | RGFFIKNENFKLDQHIATVIAQISCFDNKLPQGSP | | |
| | CSPVITNLITHSLDIRLASLAKKHKCTYTRYADD | | |
| | ITFSTRLSEFPAQIMWHDSTTYRAGKALRKEISR | | |
| | SGFSINNSKTRIQYKDSRQNVTGLVVNKKPNIK | | |
| | QEYWRLVRAKCNSGGSKRTADG | | |
| 8115 | AEFRTKLIELLKEFRDCFAWEYYEMPGLSRSIVE | ABA93011.1 | Oryza sativa |
| | HRLPIKPGVRPHQQPPRRRKADMLEPVKAEIKR | | Japonica |
| | LYDAGYNQIFMAEEDIHKTAFRCPGAIGLFEWV | | |
| | VMTFGLKSAGAMYQRAMNYIYHDSIGWLVEV | | |
| | YIDDVVVKSKEIGDHIANLRKFLRFLVHERGIEV | | |
| | TQRSVNAIKKIQPPENKTKLQEMIGKINFVRRFIS | | |
| | NLLGRLRHYLLSNECTVICKADVVKYMLSAPIL | | |
| | KGRVGKWIFSLTESDLRYESPKAIKGQAVADFI | | |
| | VEHHDDS | | |
| 8116 | SSGSIEMSIDHIVQKRGAPGYDKMQPEELPAYW | HBZ63715.1 | Lachnospiraceae |
| | AKHGERIKETIQNGSYVPRPISIHYIPKADKTKK | | bacterium |
| | RKLGIPCIIDRMILYAIQSVMTPYFEEEFSDRSYA | | |
| | FRKGKGCHDALFACLLELNRGAEYVVDLDIKSF | | |
| | FDKVNHTLLFELLDKKIEDPYLLLLLKKYIRTKA | | |
| | VCGKTFYINRIGLPQGTAISPILANMFLNSFDKH | | |
| | LEKMEIRFVRYADDIVIFCHNKEDAHYLLSDAE | | |
| | SYLRYKLKLRLNQEKTKIVRPWELEYLGYSFSA | | |
| | ASNGNMFFSLGEKTKQHMSGGSKRTADG | | |
| 8117 | KYLVEVQDEVKPRGVLNIIPKQDNFRAIVSIFPD | XP_008199629 | Tribolium |
| | SARKPFFKLLTSKIYKVLEEKYKTSGSLYTCWSE | | castaneum |
| | FTQKTQGQIYGIKVDIRDAYGNVKIPVLCKLIQS | | |
| | IPTHLLDSEKKNFIVDHISNQFVAFRRKIYKWNH | | |
| | GLLQGDPLSGCLCELYMAFMDRLYFSNLDKDA | | |
| | FIHRTVDDYFFCSPHPHKVYDFELLIKGVYQVN | | |
| | PTKTRTNLPTH | | |
| 8118 | EFRTKLIELLKEFRDCFAWQYYEMPGLSRSIVEH | XP_0241905 | Rosa chinensis |
| | RLPTEPGVRPHQQPPRRCKADMLEPVKAEIKCL | 73 | |
| | YDASFIRRCRYAEWVSSIVPVIKKNGKERVCIDF | | |
| | RDLNKATPKDEYPMPVADQLVDAASGYKILSF | | |
| | MDGNVGYNQIFMAEEDIHKTAFRCPSAIGLFEW | | |
| | VVMTFGLKSAGATYQRAMNYIYHDLISWLVEV | | |
| | YIDDVVVKSKEIEDHIADLRKVFERTRKYGLKM | | |
| | NPTKCAFGVSAGQFLGFLVHERGIEITQRSINVI | | |
| | KMIKPPEDKTELQEMIGKINFVRRFISNLSGRLEP | | |
| | FTPLLRLKADQQSTWGAEQQKALDNIKEYLSSP | | |
| | PVLIPPQKGIPFWLYLSAGDKSIGSVFIQKLEGKE | | |
| | RADVVKYMLSAPILKGRIGKWIFSLTEFDLWYE | | |
| | SQKAIKGQAIANFIVDHRDDS | | |
| 8119 | VAVSDIRVVQEFQDVFQSLQGLPPSQSDPFTIEL | XP_013739312 | Brassica napus |
| | EPGTAPLSKAPYRMAPAEMAELKKQLKDLLGK | | |
| | GFIRPSTSPWGAPVLFVKKKDGSFRLCIDYRELN | | |
| | RVTVKNRYPLPRIDELLDQLRGATCFSKIDLTSG | | |
| | YHQIPIAEADVRKTAFRTRYGHFEFVVMPFGLT | | |
| | NAPAVFMRLMNSVFQEFLDEFVIIFIDDILVYSK | | |
| | SPEEQEVHLRRVMEKLREQKLFAKLSKCSFWQ | | |
| | REMGFLGHIVSAEGVSVDPEKIEAIRDWPRPTN | | |
| | ATEIRSFLGWAGYYRRFVKGFASMAQPMTKLT | | |
| | GKDVPFVWSQECEEGFVSLKEMLTSTPVLALPE | | |
| | HGQPYMVYTDASRVGLGCVLMQHGKVIAYAS | | |
| | RQLMKHEGNYPTHDLEMAAVIFALKIWRSYLY | | |
| | GGKVQVFTDHKSLKYIFTQPELNLRQRRWMEL | | |
| | VADYDLEIAYHPGKANVVVDALSRK | | |
| 8120 | RKLENTLESETELKRTLDKLYSKTKEHMEKKTR | WP_234449435 | Staphylococcus |
| | IKHTSLLEIAMSKPNIVTAIHSLKSNKGSMTPGV | | aureus |
| | DGKTIQDYLRLSEEKLIELIRGRLTNFKAHLIKR | | |
| | VFIPKANGGQRPLGIPTIEDRIIQQMMKQVLEPV | | |
| | LEAQFFKYSFGFRPERTTYHALERVKVLVHNTG | | |
| | YHWIVEGDIRQFFDKVNHRILIKKLWSMGIKDR | | |
| | RILCLITEFLKAGIFKNIIRNDNGTPQGGILSPLLA | | |
| | NVYLHSFDKWVAKQFEEFTTRHEYSKHDHKLR | | |
| | GLKSSNLKPGYLIRYADDWVLVTNNKSHAYRW | | |
| | KTVIKNFLQKELKLELSEEKTRITNIRHKPIEFLG | | |
| | FKYKVVLKGVKGKKKKDKKTRYISQITPSDKKI | | |
| | KRKVKELRATLTSLGKRLSHDKLSNAQLILAYN | | |
| | SKVRGLINYYSYATESPIMDREGYKLRKKTFNL | | |
| | LSRRGGVLHPINKCINLADKYPKRTQKTLAIKTE | | |
| | VGYIGIMHLNLTKMNENLYKQKVQNETPYSPS | | |
| | GRKLIERRKGTKEFSVRLDEITSLSLLEKVRKKL | | |
| | VNSPRYNFEYFMNRGYAFNRDRGKCRITGVPL | | |
| | GKHNLHVHHINPNLPLEEVNKLPNLACVDKEIH | | |
| | KAIHNEVDMSSILNNKEINKLKRFRNKIHAI | | |
| 8121 | SSGSKLEYKKVRKLNKLLINSFYNWVVQIHQVA | NP_001018800 | Schizosaccharom |
| | PEGKDNSRGDGKTLRIWNKGRVLNTKELVEWR | | yces pombe |
| | TRAIEVWRRNQSYQPKKLKRIYIPKPDGTERPLS | | |
| | IPTWFDQIIQAIIGNVVECQVESIIEANDLKAYGF | | |
| | RKGFKTADAIQGLQGYALTGKKQKLVEFDRSK | | |
| | FYETIPDDKLLAVLENVDRYTRNNIEKMLKNES | | |
| | LDLKGIVTKPEMGTPQGGNISPILANLYASTQIM | | |
| | LPFKKESQSKLTMYADDGIIICDNKENPEMALA | | |
| | KLEAIAEKAGLKLKKDKTKIIDGDRFNFLGYEIT | | |
| | RGKGIRLQKDMIKKCQKDGSGGSKRTADG | | |
| 8122 | SSGSFGIDIENATFKIVVINDKNNLSGTKKRRVIH | XP_039686367 | Medicago |
| | MPSPEMRIIHKRLIRWIRGQKRLVPISASGSRPG | | truncatula |
| | DSVFKSVFIHKKTRKHSYVSNGYTHIDTIGWFPR | | |
| | HFFCLDIKDAFPSVSVSKMTEALLFAGLDPNDY | | |
| | SYNKVISILERYCFTKEDGLIVGANASPDLFNIY | | |
| | AEYFLDRNLRRYCHEHSLVYTRYLDDLIFSSNK | | |
| | TIGKRKRKSIYKFIDESGLKVNEKKTKIYDLRKG | | |
| | CAVINGIGVNEKGRMFIPRQYLDTLRGYMNSGG | | |
| | SKRTADG | | |
| 8123 | SSGSFSANHCTAEDVANLFNYLNSHGEGNVERE | HCJ67074 | Elusimicrobia |
| | MLLKVIAEPEERKVLQEVKCLIDRYYPKRKKNP | | bacterium |
| | LEGIPKIKQFVTRFRNKERHYRSFAIPKRNGGRR | | |
| | IIEAPTQELANIQRLILKKILYRNQIWNSNSSVHG | | |
| | FVPGRNILSNASLHKEATVIVRIDLKDAFRNTKE | | |
| | EMLVKHLKEYFTEKGAKILVRLCTYKGHLPQG | | |
| | APSSGMLLNFVLGELDGKLKKIAGFMGWRYSR | | |
| | YADDLTFSCVEFNKHTVGIGKLIERVKSMIKDY | | |
| | SYRVNEEKIRVFKKNRAMRVTGLVLNSGKPTIS | | |
| | RKFRRNVRAKVHSGGSKRTADG | | |
| 8124 | FEVELTQENYRLPIRNYPLPPGKMQAMNDEINQ | NP_001018800 | Schizosaccharom |
| | GLKSGIIRESKAINACPVMFVPKKEGTLRMVVD | | yces pombe |
| | YKPLNKYVKPNIYPLPLIEQLLAKIQGSTIFTKLD | | |
| | LKSAYHLIRVRKGDEHKLAFRCPRGVFEYLVMP | | |
| | YGISTAPAHFQYFINTILGEAKESHVVCYMDDIL | | |
| | IHSKSESEHVKHVKDVLQKLKNANLIINQAKCE | | |
| | FHQSQVKFIGYHISEKGFTPCQENIDKVLQWKQ | | |
| | PKNRKELRQFLGSVNYLRKFIPKTSQLTHPLNKL | | |
| | LKKDVRWKWTPTQTQAIENIKQCLVSPPVLRHF | | |
| | DFSKKILLETDASDVAVGAVLSQKHDDDKYYP | | |
| | VGYYSAKMSKAQLNYSVSDKEMLAIIKSLKHW | | |
| | RHYLESTIEPF | | |
| 8125 | ELVEHRLPIKPGFRPYKQPPRHFNPLLYDRVKEE | XP_039686367 | Medicago |
| | IDRLLKAGFIRPCRYAEWVSSIVSVEKKGSGKIR | | truncatula |
| | VCIDFRDLNKATPKDEYPMPIADMMINDASGH | | |
| | KNAGATYQRAMNLIFHDLLGIILEIYIDDIVVKS | | |
| | DGMEGHIADLRLAFERMRQYGLKMNPLKCAFG | | |
| | VSAGRFLGFMVHERGVEIYPKKIEKIRDFKAPIC | | |
| | KKEVQKLLGMVNYLRRFIFNLAGKIDAFVPILH | | |
| | LKKEADFTWGAKQQEAFEELKRYLSTPPVVRA | | |
| | PKAGKPIRLYIASEDKVIGAVLTQEEDGKVYIIT | | |
| | YLSRHLLDAETRPILSGRIGKWAYALIKYDLAY | | |
| | EPLKSMRGQIACDFIVDHHVDIAYEEDVCLVEVI | | |
| | PWKIYFDGSSCKEGQGIGVVLFSPNGMCYEASV | | |
| | RLEYYCTNNQAEYNALLFDLQVMEMVGAKYV | | |
| | EAFGDSELVVQQVAEVYKCLDGSLNRYLDSCL | | |
| | DIIANFDNFAIRHIARHDNSRANDLAQQASGYD | | |
| | VK | | |
| 8126 | SEFRTKLIELLKEYRDCFAWEYYEMPGLSRSVV | ABF96295.1 | Oryza sativa |
| | EHRLPIKPGIRPYQQPPRRCKADMLEVVKAEVK | | group |
| | HLYDAGFIHPCRYAEWVSNIVPVIKKNGKVRVY | | |
| | IDDEVVISKEIEDHIADLRKVFERTRKYGLKMNP | | |
| | TKCAFGKLEPFTPLLRLKADQKFTWGAEQQKA | | |
| | LDNIKKYLSSPPVLIPPQKGISFRLYLSAGDKSIG | | |
| | SVLIQELERKERAIFYLSRRLLDAETRYSPVEKL | | |
| | CLCLYFSCTKLRHYLLSNECTVICKADVVKYML | | |
| | SAPILKGRVGKWIFALTEFDLRYESPKTIKGQAI | | |
| | ADFIMDHRDDS | | |
| 8127 | DTDHRTDKVWVLGIQRKLYQWSKANPDDQWR | WP_0109679 | Sinorhizobium |
| | DMWGWLTDLRVLRHAWQRVASNKGGRTAGV | 89 | meliloti |
| | DGMTVGRIRNRSEHRFLVDLQADLRSGAYRPSP | | |
| | ARRKLIPKAGKPGQFRPLGIPTIRDRVVQGAAKI | | |
| | LLEPIFEAQFWHVSYGFRPGRNTHGALEYIRRA | | |
| | ALPQKRDEDTRRNRLPYPWVIEGDIKGCFDNIN | | |
| | HHHLLERMRKRIGDRRVVRLVGLFLKAGVLTE | | |
| | DQFLRTDAGTPQGGIISPLLANIALSAIEERYER | | |
| | WTYHRKKTQARRKSNGVAAAASARDSDRIAG | | |
| | RCVYLPVRYADDFVVLVSGSLEEAMAEKSALA | | |
| | DYLIKTTGLTLLPEKTKVTAMTEGFEFLGFRFSV | | |
| | HWDKRYGYGPRVEIPKAKAANLRHKVKQLTQ | | |
| | RDSISVSLGEKLRGVNAITSGWANYYRYCVGA | | |
| | GRVFVALDWYIGLRLYCWLHKKRPKATPSELW | | |
| | GSKQPSRRRATRRVWREGSVEQHVLGWTPVDR | | |
| | YRLAWMDMPDFAMSSGEPDA | | |
| 8128 | SPVLAELKEQGIVIPTHSPFNSPVWPVRKPNGK | NP_989963 | Gallus gallus |
| | WRLTIDYRRLNANTGPLTAAVPNISELIAAIQEQ | | |
| | AHPFMATIDVKDMFFMVPLHPDDQLRFAFTWE | | |
| | GQQYTFTRLPQGFKHSPTLAHYALAKELEQIPL | | |
| | EEGVRLYQYIDDILIGGDHLTPVKIMHDKIIKRL | | |
| | EELGLTIPPDKIQSPAAEVKFLGIWWKGGMACIP | | |
| | QDTLSALDQLKMPENKKELQHALGLLVFWRKH | | |
| | IPDFSIIARPLYDLLRKGVSWGWTPVHEEALQLL | | |
| | IFEAITHQSLGPIHPSDPVQIEWGFAHSGLSIHLW | | |
| | QKGPEGPIRPIGFYSRSFKDAEKRYSQLEKGLFV | | |
| | VSLALREAERTIRQQPIILRG | | |
| 8129 | SSGSNVRSIMPLSKGKSLLHRTFTTANLDSSLKT | KJR40057.1 | Candidatus |
| | LPNSSPGPDTITTDDLKKAGDQFLDKLKNNIVN | | Magneto |
| | GNYKQGKTKQYRIPKNDDTFRYIYVLNTTDRL | | ovumchiemensis |
| | VHKTIADYISPIVDNIISNSAYAYRRGLNTKGAA | | |
| | NALNNALKEGYTSGIKADISEFFDSINISALSMMI | | |
| | DSLFPFEPLADFINGILENNTRDGIKGILQGSPLSP | | |
| | LLSNLYLTRFDSDMESKGFFKLIRYADDFVLLL | | |
| | KTASSYEETIKHVEDSLSTLGLKLKPEKTTEITQ | | |
| | GKAINFLGYVITDETIAKPSGGSKRTADG | | |
| 8130 | QPLLQFPEGKVRNFQRKLYVKAKQEKTFRFYSL | WP_0666659 | Desulfotomaculum |
| | YDKLYREDVLQYAWQQCRANKGAPGADGQSF | 84 | copahuensis |
| | KDIEEKVGVERFLKEIAEELRNGTYRPMPVRRV | | |
| | YILKPDGSQRPLGIPTIKDRIAQMACLTVIQPIFE | | |
| | ADFLDCSYGFRPKRNAHQAIGAITENIKQGFTA | | |
| | VYDADLTKCFDSIQHRLIMDSLAERITDGKVLR | | |
| | LIKGWLEAPIVEPGGPKQGRKNYQGTPQGGVIS | | |
| | PLLANIVLNRLDRLWHRPGGPRERYNARLVRY | | |
| | ADDFVVLARFIGEPIKNELESIITSMGLNLNEKK | | |
| | TRILDLNKGDILNFLGYSIRISRDKNRRITIKPSD | | |
| | KAIARLRDKIREIISRERLYHGLKGIIAEINPVLRG | | |
| | WKQYFKLTNVSRIFSGLNFYITARFYRVGRKTS | | |
| | QRYSKIFKPGVYVTLRKMGLYCLATD | | |
| 8131 | WFADEPRHTRGGSRMADLYRQVRLMKTLSSA | WP_1541004 | Pseudomonas |
| | WRVVRASCMQSSSSEIRNEAIEFEADSFRQLKSI | 73 | aeruginosa |
| | QSKLQKKKFEFLPQHGIAKKRPGKSSRPLVIAPI | | |
| | PNRIVQRAILDVLQDNVAYVQEILKVETSFGGIK | | |
| | GKNVALAIAAINKAFSNGVTHYVRSDIPSFFTKV | | |
| | QRAKVVDALAKNIDDVDMVNLFSAAIETTLGN | | |
| | LTDLQRRGLESIFPLSHDGVAQGSPLSPLIANIYL | | |
| | AEFDREMNREGLACIRYIDDFVIMAASEKQVM | | |
| | KGFRAAKAVLRRQGLQVYSPDDDPLKASKGDV | | |
| | RDGFDFLGCYVKPGLVQPSKFARNRLLEKIDSG | | |
| | GSKRTADG | | |
| 8132 | RAAGIDGITVDLFTGIAREQIHQLYRQMRQERY | WP_0884289 | Halomicronema |
| | VARPAKGFYLAKQKGGHRLIGIPTVRDRIVQRY | 78 | hongdechloris |
| | LLQSIYPSLENAFSDAVFAYRPGLSIYAAVKRV | | |
| | MERYRYQPTWVIKADIQQFFDQLSWPLLLHQL | | |
| | DQLSLPATWVQWIEQQLKAGIVVSGQFYQPGQ | | |
| | GVLPGSILSGALANLYLNDFDRHCLEADIPLVR | | |
| | YGDDCVAVCQSYLEASRSLALMQDWIEGLSLS | | |
| | FHPEKTTIIPPGQAFVFLGHRFRNGTVEGPARQK | | |
| | AEGRR | | |
| 8133 | DTSNLMEQILSSDNLNRAYLQVVRNKGAEGVD | WP_1441257 | Dorea |
| | GMKYTELKEHLAKNGETIKGQLRTRKYKPQPA | 33 | formicigenerans |
| | RRVEIPKPDGGVRNLGVPTVTDRFIQQAIAQVLT | | |
| | PIYEEQFHDHSYGFRPNRCAQQAILTALNIMND | | |
| | GNDWIVDIDLEKFFDTVNHDKLMTLIGRTIKDG | | |
| | DVISIVRKYLVSGIMIDDEYEDSIVGTPQGGNLS | | |
| | PLLANIMLNELDKEMEKRGLNFVRYADDCIIMV | | |
| | GSEMSANRVMRNISRFIEEKLGLKVNMTKSKV | | |
| | DRPSGLKYLGFGFYFDPRAHQFKAKPHAKSVA | | |
| | KFKKRMKELTCRSWGVSNSYKVEKLNQLIRGW | | |
| | INYFKIGSMKTLCKELDSRIRYRLRMCIWKQWK | | |
| | TPQNQEKNLVKLGIDRNTARRVAYTGKRIAYV | | |
| | CNKGAVNVAISNKRLASFGLISMLDYYIEKCVT | | |
| | C | | |
| 8134 | SSGSQLRVEIRGRRSQPIISSWVSLLESTLFTVSPS | WP_1574472 | Catenovulum |
| | TTQLSPTHKSLSYPNYNFIDHIDADLSPHMTVRH | 74 | agarivorans |
| | ILAADLGISVQLISRILANKTQYYRSFEIIRKNGN | | |
| | KRLIEAPRTYLKVLMRYINHHLLTGLAIHDSVH | | |
| | SYRQGKSFLTNAQIHVAKQYVFNLDIENYFGCI | | |
| | NKRQVRELFSINDFTASAATLLSELCTFNDRLPQ | | |
| | GAPTSPIISNAILFKIDQSMHRYCEKNNLCYTRY | | |
| | SDDITLSGNSRQSIVKAKSRLIAMIHGAGFKIND | | |
| | KKTRLMPYHKQQLVTGVVVNKEATPARNELRR | | |
| | IRAKFHSGGSKRTADG | | |
| 8135 | ALLERILARDNLITALKRVEANQGAPGIDGVST | WP_0135228 | Geobacillus |
| | DQLRDYIRAHWSTIHAQLLAGTYRPAPVRRVEI | 81 | sp. Y412MC52 |
| | PKPGGGTRQLGIPTVVDRLIQQAILQELTPIFDPD | | |
| | FSSSSFGFRPGRNAHDAVRQAQGYIQEGYRYVV | | |
| | DMDLEKFFDRVNHDILMSRVARKVKDKRVLKL | | |
| | IRAYLQAGVMIEGVKVQTEEGTPQGGPLSPLLA | | |
| | NILLDDLDKELEKRGLKFCRYADDCNIYVKSLR | | |
| | AGQRVKQSIQRFLEKTLKLKVNEEKSAVDRPW | | |
| | KRAFLGFSFTPERKARIRLAPRSIQRLKQRIRQLT | | |
| | NPNWSISMPERIHRVNQYVMGWIGYFRLVETPS | | |
| | VLQTIEGWIRRRLRLCQWLQWKRVRTRIRELRA | | |
| | LGLKETAVMEIANTRKGAWRTTKTPQLHQALG | | |
| | KTYWTAQGLKSLTQRYFELRQG | | |
| 8136 | FTQEHLHFAWLQVCAGSKTAGVDGISVELFES | WP_0151136 | Nostoc |
| | MATEQLQNLVYQLNNETYTASPAKGFYIPKKN | 54 | sp.PCC7107 |
| | GDKRLVGIPTVRDRIIQRLLLDELYFPLEGTFLD | | |
| | CSYAYRPGHNILQAVQHLYGYYQYQPKWIIKA | | |
| | DVADFFDNLSWALLLTFLEKLSLEPSVLQLIEQQ | | |
| | LQSGMIIAGQYRNFGKGVLQGGILSGALANLYL | | |
| | TNFDKKCLSQGINLVRYGDDFVIACNSWQEAN | | |
| | RILDKITVWLGEVYLTLQSEKTQIFTPNDEFTFL | | |
| | GYRFAGGEVYAPPPPKPVLKGEWVINDSGNPYF | | |
| | RTKPRPKKPVSHPPKACSIDKPINFPRASLSHYW | | |
| | QETMTTKL | | |
| 8137 | TSSRKEQGQQKTLSRGSLQEEVVNTQGTVRAQS | WP_0834303 | Alicyclobacillus |
| | SYPAQVRSDTCGTEYTLLDEMLKLDNMMAAL | 65 | macrosporangiidus |
| | KRVEQNKGAAGVDEVDVKSLRPYLKEHWFRIR | | |
| | EELLEGTYKPQPVRRVEIPKSDGGVRLLGIPTLV | | |
| | DRLIQQGLAQVLTPIFDPNFSNSSYGFRPNRSTH | | |
| | QAVKQAKQYIEDGYRHVVDLDLEKFFDRVNHD | | |
| | ILMARVTRKVKDKRVLKLIRAYLNAGVMANG | | |
| | VCVRSEQGVPQGGPLSPILSNIILDDLDKELERR | | |
| | GHRFVRYADDCNIYVKTVRAGQRVMEGVKRF | | |
| | VEDELKLKVNEQKSAVDRPWKRKFLGFSFTPER | | |
| | KTRTRIAPKARAKFEDKVRELTSRSRSMSMAKR | | |
| | IDQLNVYLRGWMGYFRLADTRSVIESLDQWTR | | |
| | RRLRMCYLKQWKKPKSVYRNLVKLGLSADFA | | |
| | RRISGSGKGYWRLSNTPQMNKALGLAFWANQ | | |
| | GLLSLVHLYDKHRSVS | | |
| 8138 | LNQILARPNMIQALKRVEANKGRHGVDMMPV | WP_0108613 | Lysinibacillus |
| | QTLRQHILENWESIKAQILTGTYEPQPVRRVEIP | 99 | sphaericus |
| | KPDGGVRLLGIPTVTDRLIQQAISQILSKEYDQT | | |
| | FSDNSYGFRPNRSAHDAVRKAKGYMKEGYRW | | |
| | VVDMDLEKFFDKVNHDRLMATLAKRIHDKSLL | | |
| | KLIRKYLQAGVMINGVVSSTEEGTPQGGPLSPL | | |
| | LSNIVLDELDKELEKRGHKFVRYADDCNIYVKS | | |
| | KRAGDRTIASVQRFVEGKLRLKMNESKSAVDR | | |
| | PWNRKFLGFSFSHHKEPKVRVAKTSLQRMKKK | | |
| | IREITSRKKPVPMEHRIEKLNQYLIGWCGYFALA | | |
| | DTPSIFSRLDGWIKRRLRMCLWKNWKKPRTRV | | |
| | RNLIRLKVPYGKAYEWGNTRKGYWRISKSPILH | | |
| | RTLGNSYWGSQGLKSLQSRYESLRYSS | | |
| 8139 | WEQVWERENLLAALKRVERNGGAPGIDGMTV | WP_0157390 | Ammonifex |
| | EELRPYLREHWLEIRETLDQQSYQPSPVRRVEID | 74 | degensii |
| | KPEGGVRLLGIPTVLDRFLQQAMAQVLTPLFEP | | |
| | QFSPASYGFRPGRSAHEAVKQAQEYVQAGYEW | | |
| | VVDIDLERYFDQVNHDMLMARVARVVADKRV | | |
| | LKEIRAYLKSGVMVNGVVQDTGEGTPQGGPLS | | |
| | PLLSNIMLDDLDKELEKRGHKFVRYADDCNVY | | |
| | VRTQRAGERVMESVKAYLEKKLKLKVNPKKSK | | |
| | VERATRVKFLGFSFYERNGEVRVRVASQSVARF | | |
| | RKKLRGLTKRTRSGKLEEVIETINGYLMGWMA | | |
| | YYRLADTPSVFAGLDSWIRRRLRQMIWKRWKR | | |
| | GKTRYRELVKLGVPRGRAALGAVGKSPWHMS | | |
| | KTPVVNEALSNAYLRNSGLKSLKARYQELRVA | | |
| 8140 | ERILSRENLIQALERVEKNKGSYGVDEMDVKSL | WP_0108964 | Alkalihalobacillus |
| | RLHLHENWTSIRNEIIEGSYFPKPVRRVEIPKPNG | 06 | halodurans |
| | GVRKLGIPTVMDRFLQQAIAQILTQLYDPTFSER | | |
| | SFGFRPHRRGHNAVRQAKQWMKEGYRWVVDI | | |
| | DLEKFFDKVNHDRLMRKLSSRIQDPRVLQLIRR | | |
| | YLQTGVMERGLVSPNTEGTPQGGPLSPLLSNIV | | |
| | LDELDNELEKRGLKFVRYADDCNIYVRSKRAG | | |
| | LRIMESVTSFIENRLKLKVNREKSAVDRPWNRK | | |
| | FLGFSFTRGKDPKMRVSKESVKRLKQRIRELTS | | |
| | RRHSMKMSDRLRRLNRYLTGWLGYYQVVDTP | | |
| | SILAQIDAWIRRRLRMIRWKEWKTTSARQKNLV | | |
| | RLGIKKAKAWQWANSRKGYWRVAHSPIMDYA | | |
| | LNSEYWKGQGLMSLAERYQTRRWT | | |
| 8141 | LERILSRENLIQALTRVEGNKGSHGVDEMPVQN | WP_0880530 | Virgibacillus |
| | LRAHILEHWTTIRGQLEKGTYYPQPVRRVEIPKP | 30 | dakarensis |
| | NGGKRKLGIPTVMDRFLQQAIAQVLTDIYDPTF | | |
| | SQHSYGFRPKRRGHDAVREARNYIKQGYRWVV | | |
| | DMDLEKFFDKVNHDRLMRTLSQRINDSRVLKLI | | |
| | RRYLQAGVMEDGIVRPNTEGAPQGGPLSPLLSN | | |
| | IVLDELDKELEKRGLHFVRYADDCNIYVRSKRA | | |
| | GLRVMKSITKFIEGKLKLKVNEQKSAVDRPWK | | |
| | RKFLGFSFTVHKEKPKIRVSKESVQRFKQRIREL | | |
| | TSRRKSMNMKDRIEKLNRYLVGWLGYYQLAD | | |
| | TPSIFKGLDSWIRRRLRMIRWKEWKKVKTKYK | | |
| | NLMKQGINKGKAWEWANTRKAYWRIANSPIL | | |
| | HKALGDRYWSNQGLKSLYYRYQTLRWT | | |
| 8142 | AQIEEFVHVERISMLMEMILSRENLLAALKRVE | WP_0662517 | Aeribacillus |
| | QNKGSHGVDGMSVKDLRRHLYENWDSIRQSLR | 48 | pallidus |
| | EGTYKPLPVRRVEIPKPNGGVRLLGIPTVTDRFI | | |
| | QQAIAQVLTKIFDPTFSNHSYGFRPGRRGHDAV | | |
| | REAKGYIKEGYRWVVDIDLEKFFDKVNHDKLM | | |
| | GILAKTIKDQILLKVIRRYLQSGVMINGVVMET | | |
| | DMGTPQGGPLSPLLSNIMLHELDKELEKRGHKF | | |
| | VRYADDCNIYVKTKKAGIRVMNSITNFIEKELK | | |
| | LKVNKEKSAVDRPWKRKFLGFSFTLNKTPKVRI | | |
| | ANESVKRLKNKIREITSRSKPYPMEKRIEKLNKY | | |
| | LMGWCGYFALAETPSKFKELDEWIRRRLRMCL | | |
| | WKEWKTPKTRIRKLRGLGVPSHKAFEWGNTRK | | |
| | KYWRIACSPILHKTLDNSYWKRRGLRSLFERYQ | | |
| | ALRHT | | |
| 8143 | LMDLILSRENLIAALKRVERNKGSHGIDGMSVK | WP_1972453 | Cytobacillus |
| | SLRRHLYENWDSLCDSLRKGTYQPNPVRRIEIP | 11 | firmus |
| | KPNGGVRLLGIPTVTDRFIQQAIAQILTPLFDPSF | | |
| | SEHSYGFRPNRRGHDAVRKAREYISEGYRWVID | | |
| | MDLEKFFDKVNHDKLMGILASRIQDRLVLKLIR | | |
| | KYLQAGIMINGVVYDAEEGTPQGGPLSPLLSNIL | | |
| | LDKLDKELERRGHKFVRYADDCNIYMKSKKAG | | |
| | ERVMNSITRFIEQKLKLKVNRGKSAVDRPWKR | | |
| | KFLGFSFTLNKKPKVRIANESIKRLKTKIREFTSR | | |
| | SKSIPMEVRIEKLNQYLTGWCGYFALADTPSKF | | |
| | KEFDEWIRRRLRMIEWKQWKNPRTRVRKLKGL | | |
| | GVPDQKAYEWGNSRRKYWRIASSPILHKTLDN | | |
| | SYWSNRGLKSLYQRYEFLRQT | | |
| 8144 | RSHEGQRQQKISRESLRQREAVKPSGYAGAPSS | WP_1382262 | Paenibacillus |
| | SSAQIDPSSREANNDLLERMLRGDNLRLAYKRV | 90 | algicola |
| | VQNGGAPGVDNVTVANLQAYLKTHWEAVKTE | | |
| | LLAGTYRPMPVKRVEIPKPGGGVRLLGIPTVMD | | |
| | RFLQQALLQVMTPIFDADFSRHSYGFRPGKRAH | | |
| | DAVKQAQRYMQEGFRWVVDMDLAKFFDRVN | | |
| | HDMLMARVARKVTDKCVLKLIRAYLNAGVMA | | |
| | NGVTEKTGEGTPQGGPLSPLLANILLDDLDKEL | | |
| | TERGLRFVRYADDCNIFVASKRAGERVMDSVT | | |
| | RFVEGKLKLKVNREKSAVDRPWNRKFLGFSFL | | |
| | RDKKATIRLAPQTISRFKEKVRELTNRTRSMSM | | |
| | ENRIAQLNRYLMGWIGYFRLASAKGHCEKFDQ | | |
| | WIRRRLRMCLWKQWKRVRTRIRELRALGVPE | | |
| | WACFVMANSRRGAWEMSRNTNNALPTSYWEA | | |
| | KGLKSLLSRYLELC | | |
| 8145 | SSNELHRKQKTHSRASLREIAVNTQRTAKGQSIS | WP_2210399 | Gelria sp. |
| | SAQTRISPCKDQGNNLMEKVVERSNMMAALRR | 73 | Kuro-4 |
| | VEQNKGAAGIDGVKTDELRNLLWDIWTDTKEQ | | |
| | LLTGTYRPKPVRRVEIPKPNGGVRLLGIPTVLDR | | |
| | LIQQALLQILTSIFDPTFSEASYGFRPGKRAHTAV | | |
| | RVARSYVESGYDWVIDMDIEKFFDRVNHDILM | | |
| | ARVARKVKDKRVLKLIRRYLQAGVMLGGVVV | | |
| | RTEEGTPQGGPLSPLLANIYLDDLDKELEKRGH | | |
| | KFVRYADDCNIYVKSQRAAERVMQSIREFLQK | | |
| | RLKLKVNEEKSCVDRPWNLKYLGFSMFKSKGK | | |
| | VRICLAPETIKRVKNKIREFTSRSKPIRMEDRIRR | | |
| | LNAYLGGWLGYFALADTSNDFASIDGWLRRRL | | |
| | RMCLWRQWDRVRTRLRELRALGLPEWVAHQL | | |
| | ANTRKGPWRMSHRPLHSALNNAYWEKQGLLS | | |
| | LAKRHQAICQA | | |
| 8146 | SPANRAVIDEQMDKWMKLDVIEPSKSPWAAPV | XP_0431833 | Rhizoctonia |
| | FIVYRNAKPRMVIDLRKLNESVVPDEHPIPRQEE | 11 | solani |
| | ILQSLQGCKYLSSLDALAGFTQLSIHEDDREKLA | | |
| | FRTHRGLFQFKRMPFGYRNGPAVFQRVMQNVL | | |
| | SPYLWLFTLVYIDDIVIYSKTFDEHLAHVDLVLK | | |
| | AIMESKLTLSPDKCHFGYGSILLLGQKVSRLGLS | | |
| | THKEKVQSILDLETPKNVKTLQTFLGMMVYFSS | | |
| | YIPFYSWIAHPLFQLLKKGTKWEWKAEHQNAF | | |
| | ELCKEVLTEAPVRAHAMPGRPYRVYSDACDFG | | |
| | LAAILQQVQPVEIRDLRGTRTYDRLRKAFDKGE | | |
| | KVPVLAQTISKDVKDVPEDVWASDFESTTVHIE | | |
| | RVIAYWSRVLQSAERNYSPTEREALALKEGLVK | | |
| | FQVYLEGEKVLAITDHAALQWSRTFQNVNRRL | | |
| | LSWGLVFSAF | | |
| 8147 | SSGSSYFYKERRGKIYESYYGSIVTYNTTTSKMA | WP_0147575 | Thermoanaero |
| | DSKELFSSFFKARMSLKKEFPYDEIAIKLFEYNL | 44 | bacterium |
| | EDNINRFSKEILKGYKFNTDFIGYKVPKNEKDD | | |
| | RQKVMDNIFNTIAGASFLDIIGIVIDREFSSNCCG | | |
| | NRLNKKLNTEYSYEYFWYGWYYKFMKKAFNK | | |
| | VLNKNNYYLKLDIKSFYTNINQNILYDKIIKLIPY | | |
| | KDSRLKEFINSLIKRHIPYVNNGKGLPQGSLTSG | | |
| | FLANLYLDDFDKYFISKTNDGYMRYVDDIFIFG | | |
| | KTEEQIKELGKEAENKLKDLYLEINKEKTSMGD | | |
| | KSSLKNIYYDDKELDDFQKRLSGGSKRTADG | | |
| 8148 | SSGSLRINSLKHLAHRLGFAPEVLQKAASRAEK | WP_0159038 | Desulforapulum |
| | SYKFDKIPKKSGKGFREISKPNALLKNIQKAIHK | 04 | autotrophicum |
| | LLTEIEISDNAHCGIKKRSNVTNAMNHCNKEWV | | |
| | YSMDFKNFFPNISHHQVYGLFRYELKCSPDVTSI | | |
| | LTRLCTVKGGVPQGGSMSMDIANLVSRKLDTR | | |
| | LEGLCKIHNLSYTRHCDDLNFSGKRILDTFRAK | | |
| | VEIIIKESGFPLNPDKETLIPHHHPQSVVGLRVNR | | |
| | KKPCVPRKTRREWRKEKHSGGSKRTADG | | |
| 8149 | IPNVVWKECVELLAPQLERIFKAVYEKGMYSER | 36HUJA2X0 | Citromicrobium |
| | WKEWTTVVLKKPGKPRYDTPKAWRPIALMNT | 1R | |
| | MGKILTALLTEDLKYVTEKYSLLPNTHFGGRPG | | |
| | RTTTDAIQLLTSWIKGHWRKGNVVSVLFLDIEG | | |
| | AFPNVVVSRLAHNMRRRRVPEFIVKLIEHQLRD | | |
| | RRTKLKFDDYESEWVPIDNGSGQGDPKSMLEY | | |
| | LFYNADLIDLVAGLGEELEEGENGEDAPRGSAR | | |
| | ERGTEKRDENAAAFVDDAWLGGAGATFEEAN | | |
| | ETLKDMMNRRGGAMEWSKKHNSKFEISKLVY | | |
| | MGFTRRMR | | |
| 8150 | KSAEYLNTFRLRNLGLPVMNNLHDMSKATRIS | WP_0990105 | Escherichia coli |
| | VETLRLLIYTADFRYRIYTVEKKGPEKRMRTIYQ | 51 | |
| | PSRELKALQGWVLRNILDKLSSSPFSIGFEKHQSI | | |
| | LNNATPHIGANFILNIDLEDFFPSLTANKVFGVF | | |
| | HSLGYNRLISSVLTKICCYKNLLPQGAPSSPKLA | | |
| | NLICSKLDYRIQGYAGSRGLIYTRYADDLTLSA | | |
| | QSMKKVVKARDFLFSIIPSEGLVINSKKTCISGPR | | |
| | SQRKVTGLVISQEKVGIGREKYKEIRAKIHHIFC | | |
| | GKSSEIEHVRGWLSFILSVDSKSHRRLIAYISKLE | | |
| | KKYGKNPLNKAKT | | |
| 8151 | ITPLVSFTSFAQFEQALRDSRVSAHSGASFSNSLE | WP_0142640 | Granulicella |
| | VDRLVKSARELYGRGLPPIVSSRTFSLLFGVSPR | 22 | mallensis |
| | LISAMTKSPEKYWRTFEIKKRSGKGRNIAAPRVF | | |
| | LKTVQRFLLRFVLEKIPIHPNAFGFAPGKGIFKH | | |
| | AERHLKARFVLTLDIADFFPSISWTQVRDIFANI | | |
| | GFPDGVPSLLADLCTRNKVFPQGAPTSPYLSNLI | | |
| | FLKTDEALTEAANQFEMRYSRYADDLTFSCDSQ | | |
| | PSDEARLAFEQIIRDAGFRIQHSKTRLRGPSQAR | | |
| | EVTGLLVNEKIQPSRHTRRLLRAKFH | | |
| 8152 | SLQLEEEYRLYQEREKPPEELQEWLLRFPQAWA | XP_0343693 | Arvicanthis |
| | ETGGTGMARQAPPVVIELKSGATPIGVRQYPMS | 84 | niloticus |
| | KEAREGIRPHIKRLLEQGILVPCRSPWNTPLLPV | | |
| | KKPGTNDYRPVQDLREVNKRVQDVHPTVPNPY | | |
| | NLLSTLPPTRTWYTVLDLKDAFFCLRLHPNSQP | | |
| | LFAFEWRDPESGRTGQLTWTRLPQGFKNSPTLF | | |
| | DEALHRDLAPFRANNPQVTLIIYIDDILLATETRE | | |
| | DCELGTQKILAELGELGYRVSAKKAQLCRTEVT | | |
| | YLGYTLKNGQRWLTEARKRTVTQIPTPTTPRQV | | |
| | REFLGTAGFCRLWIPGFATLAAPLYPLTKEKGK | | |
| | FIWTKEHQVAFETLKKTLLQAPALALPDLSKPF | | |
| | TLYIDERKGVARGVLTQALGPWKRPVAYLSKK | | |
| | LDPVASGWPSCLRAIAATATLIKDADKLTLGQK | | |
| | VTVVAPHALENIIRQPPDRWITNARITHYQSLLL | | |
| | TERVTFAPPAVLNPATLLPEADETPVHQCEEILA | | |
| | EEAGTWSDLTDQPWPGAETWFTNGSSFVKKGK | | |
| | RRAGAAVVDRRTVIWASSLPEGTSAQKAELIAL | | |
| | IQALKLAEGKSVNIYTDSRYAFATAHVHGAIYR | | |
| | QRGLLTSAGRDIKNKKEILDLLVAIHLPRKVAII | | |
| | HCPGHQKGTGPIEKGNQMADQMAKEAAHGPM | | |
| | TLIAKVGSRQDERALEKRALTEEEGLEYLT | | |
| 8153 | VLNLEEEYRLHEKPVPSSIDPSWLQLFPTVWAE | NP_056790 | Gibbon ape |
| | RAGMGLANQVPPVVVELRSGASPVAVRQYPMS | | leukemia virus |
| | KEAREGIRPHIQRFLDLGVLVPCQSPWNTPLLPV | | |
| | KKPGTNDYRPVQDLREINKRVQDIHPTVPNPYN | | |
| | LLSSLPPSHTWYSVLDLKDAFFCLKLHPNSQPLF | | |
| 8154 | AFEWRDPEKGNTGQLTWTRLPQGFKNSPTLFD | XP_0370593 | Peromyscus |
| | EALHRDLAPFRALNPQVVLLQYVDDLLVAAPT | 69 | leucopus |
| | YRDCKEGTQKLLQELSKLGYRVSAKKAQLCQK | | |
| | EVTYLGYLLKEGKRWLTPARKATVMKIPPPTTP | | |
| | RQVREFLGTAGFCRLWIPGFASLAAPLYPLTKES | | |
| | IPFIWTEEHQKAFDRIKEALLSAPALALPDLTKPF | | |
| | TLYVDERAGVARGVLTQTLGPWRRPVAYLSKK | | |
| | LDPVASGWPTCLKAVAAVALLLKDADKLTLGQ | | |
| | NVTVIASHSLESIVRQPPDRWMTNARMTHYQSL | | |
| | LLNERVSFAPPAVLNPATLLPVESEATPVHRCSE | | |
| | ILAEETGTRRDLKDQPLPGVPAWYTDGSSFIAE | | |
| | GKRRAGAAIVDGKRTVWASSLPEGTSAQKAEL | | |
| | VALTQALRLAEGKDINIYTDSRYAFATAHIHGAI | | |
| | YKQRGLLTSAGKDIKNKEEILALLEAIHLPKRVA | | |
| | IIHCPGHQKGNDPVATGNRRADEAAKQAALST | | |
| | RVLAETTKPQELI | | |
| | ALSLEEEYRLFEAEPPEKSPEELQNWLREFPQA | | |
| | WAETAGLGLARDQPPLMISLKASATPVSIRQYP | | |
| | MSREAHEGIKPHIRRLLDQGVLKPCQSPWNTPL | | |
| | LPVKKPGTGDYRPVQDLREVNKRVEDIHPTVPN | | |
| | PYNLLSTLPPTHVWYTVLDLKDAFFCLRLHPQS | | |
| | QLLFAFEWRDPEKGSSGQLTWTRLPQGFKNSPT | | |
| | LFDEALHADLAGFRVEHPTLTLLQYMDDLLLV | | |
| | ARSRTECMEGTRALLARLGQKGYRASAKKAQV | | |
| | CRDKVTYLGYTLSRGQRWLTGARKETIISIPPPR | | |
| | NPRQVREFLGTAGYCRLWIPGFAKLAAPLYPLT | | |
| | KPGTMFQWEEKHQKAFQQIKKALLEAPALGLP | | |
| | DLTKPFELFVDENSGFAKGVLVQRLGPWRRPV | | |
| | AYLSKKLDPVATGWPPCLRMVAAIAVLLKDAG | | |
| | KLTLGQPLTVLASHAVEALVRQPPDRWLSNAR | | |
| | MTYYQALLLDSDRVNFGPIVSLNPATLLPLPSPS | | |
| | EEHDCLQILAEAHGTRPDLTDQPLKDPDAVWY | | |
| | TDGSSFLEEGERRAGAAITTESEVVWASSLPPGT | | |
| | SAQRAELIALTQALRMAEGKKLTVYTDSRYAF | | |
| | ATAHVHGEIYRRRGLLTSAGKEIKNKKEILDLL | | |
| | KALFLPLQLSIVHCPGHQKDDSAVARGNRLADL | | |
| | TARTVASQPAGSSQLMAIQEPQPERDPVPYSPE | | |
| | DHELAKKMGSDWEQRRQAYILGDRMVMSTSH | | |
| | TRYMLR | | |
| 8155 | TLKLEDEYKLHDDPRPTPENIDQWLAKYPEAW | XP_0088533 | Nannospalax |
| | AETNGMGLAKQQPPLVISLKASVTPANVKQYP | 49 | galili |
| | MTIEAQQGIRPHIKRLLEQGILIPCQSAWNTPLLP | | |
| | VKKPGGTDYRPVQDLREVNKRVEDIHPTVPNP | | |
| | YNLLSTLPPSHAWYTVLDLKDAFFCLKLHPSSQ | | |
| | PLFAFEWKDPELGLSGQLTWTRLPQGFKNSPTL | | |
| | FDGALHQDLAAFRTQYPHLIILQYVDDILLAAET | | |
| | KEECLEGTGALLQELGQLGYRASAKKAQLCKK | | |
| | EVTYLGYQLKEGQRWLTKARQQTILSIPAPKDR | | |
| | KQVREFLGTAGFCRLWIPGFAEMAAPLYPLTKA | | |
| | SEGFTWENEHQRAFENIKQALLTAPALGLPDLN | | |
| | KPFELYVDEKTGYAKGVLTQKLGPWRRPVAYL | | |
| | SKKLDPVASGWPPCLRMVAALAVLVKDAFKLT | | |
| | LGQPLCIRAPHALESLIRQPPDRWLSNTRMTHY | | |
| | QALLLDTDRIQFGSPVALNPATLLPSTEEPDHHD | | |
| | CLQILAEVFGTRPDLKDQPLDNADYTWYTDGSS | | |
| | FLKGSQRRAGAAVTSKDKVIWAKPLPEGTSAQ | | |
| | KAELIALTQALRLAEGKSLNVYTDSRYAFATAH | | |
| | IHGEIYRRRGLDL | | |
| 8156 | TCPLSEESRLLPLTFSPDRPSTTSTPTSLTLLNHF | XP_0277130 | Vombatus |
| | KGLVPGVWAETNPFGLAGHQPPVVVQLSSTAT | 74 | ursinus |
| | PACVQQYPLTRAALLGIKPHIDRLLAAGILRPCQ | | |
| | SSWNTPLLPVRKPGSGDFRPVQDLREVNARVET | | |
| | VHPTVPNPYTLLSSLDPARTWYTVLDLKDAFFSI | | |
| | PLAPVSQPIFAFTWTDPNTGTSSQLTWTRLPQGF | | |
| | KNSPTLFGSALASDLAAFRVSYPEVTLLQYVDD | | |
| | LLLATSSEAICKDATLHLLQLLEASGYRISGKKA | | |
| | QLCSQSVVYLGFTLRSGQRLLSRGRVAAILGMP | | |
| | APRNRRGLREFLGMAGYCRLWILGFAEVAKPL | | |
| | YEALTGEPTQFVWGPRQQEAFDKLRKALSSTPA | | |
| | LSLPDLSKPFRLYVSESRAVAKGVLTQPLGPWN | | |
| | RPVAYLSKQLDPVASGWPSCLRTVAAIAVLVRE | | |
| | AAKLTFGQPLEISASHHLEQLLHSPPTRWISNSR | | |
| | LTHYQSLLLDSARISFAPPVTLNPATLLPDSPPSS | | |
| | PIHDCLDTLDSIHTSRPGLTDVPLTNPDLVLFTD | | |
| | GSSFVQEGIRRAGAAVVTPVETLWDTALPPGTS | | |
| | AQRAELIALTQALRLSAGRRVNIYTDSRYAFAT | | |
| | VHIHGYVYLQRGLLTSAGREIRNKSQIQDLLDA | | |
| | VWLPKEVAVIHVPAHTRGTDPQSLGNAAADKA | | |
| | ARAAACKPLIPAM | | |
| 8157 | TAPLEEEYRLFLEAPIQNVTLLEQWKREIPKVW | YP_223871 | Reticulo |
| | AEINPPGLASTQAPIHVQLLSTALPVRVRQYPITL | | endotheliosis |
| | EARRSLRETIRKFRAAGILRPVHSPWNTPLLPVR | | virus |
| | KPGTSEYRMVQDLREVNKRVETIHPTVPNPYTL | | |
| | LSLLPPDRTWYSVLDLKDAFFCIPLAPKSQLIFA | | |
| | FEWTDAEEGESGQLTWTRLPQGFKNSPTLFDEA | | |
| | LNRDLQGFRLDHPSVSLLQYVDDLLIAADTQAA | | |
| | CLSATRDLLMTLAELGYRVSGKKAQLCQEEVT | | |
| | YLGFKIHKGSRTLSNSRTQAILQIPVPKTKRQVR | | |
| | EFLGTIGYCRLWIPGFAELAQPLYAATRGGNDP | | |
| | LEWGEKEEEAFQSLKLALTQPPALALPSLDKPF | | |
| | QLFIEETGGAAKGVLTQALGPWKRPVAYLSKR | | |
| | LDPVAAGWPRCLRAIAAAALLTREASKLTFGQ | | |
| | DIEITSSHNLESLLRSPPDRWLTNARITQYQVLLL | | |
| | DPPRVRFKQTAALNPATLLPETDDTLPIHHCLDT | | |
| | LDSLTSTRPDLTDQPLAQAEATLFTDGSSYIPHG | | |
| | KRYAGAAVVTLDSVIWAEPLPIGTSAQKAELIA | | |
| | LTKALEWSKDKSVNIYTDSRYAFATLHVHGMI | | |
| | YKERGLLTAGGKAIKNAPEILALLTAVWLPKRV | | |
| | AVMHCRGHQKDDAPTSAGNRRADEVAREVAI | | |
| | RPLSIQATVFDAPDMP | | |
| 8158 | TVLLPPTYHKQLSCQTKNTLNIDEYLLQFPDQL | NP_045937 | Walleyedermal |
| | WASLPTDIGRMLVPPITIKIKDNASLPSIRQYPLP | | sarcoma virus |
| | KDKTEGLRPLISSLENQGILIKCHSPCNTPIFPIKK | | |
| | AGRDEYRMIHDLRAINNIVAPLTAVVASPTTVL | | |
| | SNLAPSLHWFTVIDLSNAFFSVPIHKDSQYLFAF | | |
| | TFEGHQYTWTVLPQGFIHSPTLFSQALYQSLHKI | | |
| | KFKISSEICIYMDDVLIASKDRDTNLKDTAVML | | |
| | QHLASEGHKVSKKKLQLCQQEVVYLGQLLTPE | | |
| | GRKILPDRKVTVSQFQQPTTIRQIRAFLGLVGYC | | |
| | RHWIPEFSIHSKFLEKQLKKDTAEPFQLDDQQV | | |
| | EAFNKLKHAITTAPVLVVPDPAKPFQLYTSHSE | | |
| | HASIAVLTQKHAGRTRPIAFLSSKFDAIESGLPPC | | |
| | LKACASIHRSLTQADSFILGAPLIIYTTHAICTLL | | |
| | QRDRSQLVTASRFSKWEADLLRPELTFVACSAV | | |
| | SPAHLYMQSCENNIPPHDCVLLTHTISRPRPDLS | | |
| | DLPIPDPDMTLFSDGSYTTGRGGAAVVMHRPVT | | |
| | DDFIIIHQQPGGASAQTAELLALAAACHLATDK | | |
| | TVNIYTDSRYAYGVVHDFGHLWMHRGFVTSA | | |
| | GTPIKNHKEIEYLLKQIMKPKQVSVIKIEAHTKG | | |
| | VSMEVRGNAAADEAAKNAVFLVQRVLK | | |
| 8159 | TTLVPLQEYEERLLKQTMLTGSYKEKLQSLFLK | YP_001956722 | African green |
| | YDALWQHWENQVGHRRIKPHHIATGTVNPRPQ | | monkey simian |
| | KQYPINPKAKASIQTVINDLLKQGVLIQQNSIMN | | foamy virus |
| | TPVYPVPKPDGKWRMVLDYREVNKTIPLIAAQ | | |
| | NQHSAGILSSIFRGKYKTTLDLSNGFWAHSITPE | | |
| | SYWLTAFTWLGQQYCWTRLPQGFLNSPALFTA | | |
| | DVVDLLKEVPNVQVYVDDIYISHDDPREHLEQL | | |
| | EKVFSLLLNAGYVVSLKKSEIAQHEVEFLGFNIT | | |
| | KEGRGLTETFKQKLLNITPPRDLKQLQSILGLLN | | |
| | FARNFIPNFSELVKPLYNIIATANGKYITWTTDN | | |
| | SQQLQNIISMLNSAENLEERNPEVRLIMKVNTSP | | |
| | SAGYIRFYNEFAKRPIMYLNYVYTKAEVKFTNT | | |
| | EKLLTTIHKGLIKALDLGMGQEILVYSPIVSMTK | | |
| | IQKTPLPERKALPIRWITWMSYLEDPRIQFHYDK | | |
| | TLPELQQVPTVTDDIIAKIKHPSEFSMVFYTDGS | | |
| | AIKHPNVNKSHNAGMGIAQVQFKPEFTVINTWS | | |
| | IPLGDHTAQLAEVAAVEFACKKALKIDGPVLIV | | |
| | TDSFYVAESVNKELPYWQSNGFFNNKKKPLKH | | |
| | VSKWKSIADCIQLKPDIIIIHEKGHQPTASTFHTE | | |
| | GNNLADKLATQGSYVVNINTTPSLDAELDQLLQ | | |
| | GQYP | | |
| 8160 | TTLVPLQEYQERLLKQTALPEREKKILHSLFLKY | YP_009666126 | Guenon simian |
| | DALWQHWENQVGHRRIKPHHIATGTVNPRPQK | | foamy virus |
| | QYPINPKAKPSIQIVINDLLKQGVLIQQNSVMNT | | |
| | PVYPVPKPDGKWRMVLDYREVNKTIPLIAAQN | | |
| | QHSAGILSSIVREKYKTTLDLSNGFWAHSITPES | | |
| | YWLTAFTWQGKQYCWTRLPQGFLNSPALFTAD | | |
| | VVDLLKEVPNVQAYVDDIYISHNDPKEHLEQLE | | |
| | KVFSLLLNAGYVVSLKKSEIAQYEVEFLGFNITK | | |
| | EGRGLTDTFKQKLLNITPPKDLKQLQSILGLLNF | | |
| | ARNFIPNFSELVKPLYNIIAIANGKFIQWTEENSQ | | |
| | QLQYIISVLNSAENLEERNPEVKLIMKVNTSPSA | | |
| | GYIRFYNESAKRPIMYLNYVYTKAEIKFTNTEK | | |
| | LLTTIHKGLIKALDLAMGQGILVYSPIVSMTKIQ | | |
| | RTPLPERKALPIRWITWMSYLEDPRIQFHYDKTL | | |
| | PELQNVPMVTGDEVAKTKHPSEFSMVFYTDGS | | |
| | AIKHPNINKSHSAGMGIAQVQFKPEFTVLNTWSI | | |
| | PLGDHTAQLAEVAAVEFACKKALKINGPVLIVT | | |
| | DSFYVAESANKELPYWQSNGFLNNKKKPLRHIS | | |
| | KWKSIAECIQLKPDISIIHEKGHQPTATTFHTEGN | | |
| | TLADKLATQGSYVVNSNTTPSLDAELDQLLQG | | |
| | RYP | | |
| 8161 | TVLVPLQDYQERLLKQTTLPKEQKDQLEKLFLK | YP_009513242 | Rhesus macaque |
| | YDALWQHWENQVGHRRIKPHNIATGTLAPRPQ | | simian foamy |
| | KQYPINPKAKPSIQIVIDDLLKQGVLIQQNSTMN | | virus |
| | TPVYPVPKPDGKWRMVLDYREVNKTIPLIAAQ | | |
| | NQHSAGILSSIYRGKYKTTLDLTNGFWAHPITPE | | |
| | SYWLTAFTWQGKQYCWTRLPQGFLNSPALFTA | | |
| | DVVDLLKEVPNVQAYVDDIYMSHDDPQEHLEQ | | |
| | LEKVFSILLNAGYVVSLKKSEIAQREVEFLGFNI | | |
| | TKEGRGLTETFKQKLLNVIPPKDLKQLQSILGLL | | |
| | NFARNFIPNYSELVKPLYTIVANANGKFISWTEE | | |
| | NSNQLQYIISVLNQADNLEERNPETRLILKVNSS | | |
| | PSAGYIRYYNEGSKRPIMYVNYVFSKAEVKFTQ | | |
| | TEKMLTTMHKGLIKAMDLAMGQEILVYSPIVS | | |
| | MTKIQKTPLPERKALPVRWITWMTYLEDPRIQF | | |
| | HYDKTLPELQQTPSVTEDVIAKTKHPSEFAMVF | | |
| | YTDGSAIKHPDINKSHSAGMGIAQVQFQPEYKV | | |
| | IHQWSIPLGDHTAQLAEIAAVEFACKKALKISGP | | |
| | VLIVTDSFYVAESANKELSYWKSNGFLNNKKKP | | |
| | LKHVSKWKSIAECLQLKPDITIIHEKGHQQPMTT | | |
| | LHTEGNNLADKLATQGSYVVHCNTTPSLDAEL | | |
| | DQLLQGHNP | | |
| 8162 | TVLVPLHEYQERLLQQTALPKEQKELLQKLFLK | YP_009508556 | Japanese |
| | YDALWQHWENQVGHRRIKPHNIATGTLAPRPQ | | macaque simian |
| | KQYPINPKAKPSIQIVIDDLLKQGVLIQQNSTMN | | foamy virus |
| | TPVYPVPKPDGKWRMVLDYREVNKTIPLIAAQ | | |
| | NQHSAGILSSIYRGKYKTTLDLTNGFWAHPITPE | | |
| | SYWLTAFTWQGKQYCWTRLPQGFLNSPALFTA | | |
| | DVVDLLKEIPNVQAYVDDIYISHDDPQEHLEQL | | |
| | EKIFSILLNAGYVVSLKKSEIAQREVEFLGFNITK | | |
| | EGRGLTDTFKQKLLNITPPKDLKQLQSILGLLNF | | |
| | ARNFIPNYSELVKPLYTIVANANGKFISWTEDNS | | |
| | NQLQHIISVLNQADNLEERNPETRLIIKVNSSPSA | | |
| | GYIRYYNEGSKRPIMYVNYIFSKAEAKFTQTEK | | |
| | LLTTMHKGLIKAMDLAMGQEILVYSPIVSMTKI | | |
| | QRTPLPERKALPVRWITWMTYLEDPRIQFHYDK | | |
| | SLPELQQIPNVTEDVIAKTKHPSEFAMVFYTDGS | | |
| | AIKHPDVNKSHSAGMGIAQVQFIPEYKIVHQWS | | |
| | IPLGDHTAQLAEIAAVEFACKKALKISGPVLIVT | | |
| | DSFYVAESANKELPYWKSNGFLNNKKKPLRHV | | |
| | SKWKSIAECLQLKPDIIIMHEKGHQQPMTTLHTE | | |
| | GNNLADKLATQGSYVVHCNTTPSLDAELDQLL | | |
| | QGHYP | | |
| 8163 | TVLVPLQEYQERLLKHTALPKEQVKQLEKLFLK | YP_009508551 | Eastern |
| | FDALWQHWENQVGHRRIKPHNIATGILTPRPQK | | chimpanzee |
| | QYPINPKAKPSIQIVIDDLLKQGVLIQQNSIMNTP | | simian foamy |
| | VYPVPKPDGKWRMVLDYREVNKTIPLIAAQNQ | | virus |
| | HSAGILSSIYRGKYKTTLDLTNGFWAHPITPESY | | |
| | WLTAFTWQGKQYCWTRLPQGFLNSPALFTADV | | |
| | VDLLKEVQNVQAYVDDIYISHDDPQEHVEQLE | | |
| | KVFSILLNAGYVVSLKKSEIAQREVEFLGFNITK | | |
| | EGRGLTDTFKQKLLNITPPKDLKQLQSILGLLNF | | |
| | ARNFIPNYSELVKPLYTIVANANGKFITWSEENS | | |
| | NQLQRIISVLNQAENLEERNPETRLIIKINSSPSA | | |
| | GYIRYYNEGSKRPIMYVNYVFSKAEMKFTHTE | | |
| | KLLTTMHKGLIKAMDLAMGQEILVYSPIVSMTK | | |
| | IQKTPLPERKALPVRWITWMTYLEDPRIQFHYD | | |
| | KSLPELQQIPNVTEDVIAKTKHPSEFSMVFYTDG | | |
| | SAIKHPDVNKSHSAGMGIAQAQFQPEYKVLHQ | | |
| | WSIPLGDHTAQLAEIAAVEFACKKALKVSGPVL | | |
| | IVTDSFYVAESANKELSYWKSNGFLNNKKKPLK | | |
| | HVSKWKSIAECLQLKPDIVIIHEKGHQPSMTTLH | | |
| | TEGNNLADKLATQGSYVVHCNTTPSLDAELDQ | | |
| | LLQGHNP | | |
| 8164 | TILVPLQEYQDRILNKTALPEEQKQQLKALFTK | NP_056803 | Simian foamy |
| | YDNLWQHWENQVGHRKIRPHNIATGDYPPRPQ | | virus |
| | KQYPINPKAKPSIQIVIDDLLKQGVLTPQNSTMN | | |
| | TPVYPVPKPDGRWRMVLDYREVNKTIPLTAAQ | | |
| | NQHSAGILATIVRQKYKTTLDLANGFWAHPITP | | |
| | DSYWLTAFTWQGKQYCWTRLPQGFLNSPALFT | | |
| | ADAVDLLKEVPNVQVYVDDIYLSHDNPHEHIQ | | |
| | QLEKVFQILLQAGYVVSLKKSEIGQRTVEFLGF | | |
| | NITKEGRGLTDTFKTKLLNVTPPKDLKQLQSILG | | |
| | LLNFARNFIPNFAELVQTLYNLIASSKGKYIEWT | | |
| | EDNTKQLNKVIEALNTASNLEERLPDQRLVIKV | | |
| | NTSPSAGYVRYYNESGKKPIMYLNYVFSKAELK | | |
| | FSMLEKLLTTMHKALIKAMDLAMGQEILVYSPI | | |
| | VSMTKIQKTPLPERKALPIRWITWMTYLEDPRIQ | | |
| | FHYDKTLPELKHIPDVYTSSIPPLKHPSQYEGVF | | |
| | CTDGSAIKSPDPTKSNNAGMGIVHAIYNPEYKIL | | |
| | NQWSIPLGHHTAQMAEIAAVEFACKKALKVPG | | |
| | PVLVITDSFYVAESANKELPYWKSNGFVNNKKE | | |
| | PLKHISKWKSIAECLSIKPDITIQHEKGHQPINTSI | | |
| | HTEGNALADKLATQGSYVVNCNTKKPNLDAEL | | |
| | DQLLQGNNV | | |
| 8165 | TVLVPLEQYKERILKETALEGQFKQQLQNILSTF | YP_ | Simian foamy |
| | DTLWQHWENQVGHRKIPPHNIATGTHPPRPQK | 009508888 | virus |
| | QYPINPKAKESIQIVINDLLKQGVLIQQNSIMNTP | | Pongopygmaeus |
| | VYPVPKPDGRWRMVLDYREVNKTIPLIAAQNQ | | pygmaeus |
| | HSAGILASIYRGTYKTTLDLANGFWAHPITPNSY | | |
| | WLTAFTWQGKQHCWTRLPQGFLNSPALFTADV | | |
| | VDLMKHIPNVQVYVDDLYLSHDDPQEHLQVLQ | | |
| | QVLHILHDAGYVVSLKKSAIAQKVVEFLGFNIT | | |
| | KTGRGLTDAFKEKLLNISPPQNLKQLQSILGLM | | |
| | NFARNFIPNYAERVKPFYSLISTAKSNNILWNDE | | |
| | LTSQLQELITLLNQADNLEERKPTTRLIIKVNSSS | | |
| | HAGYIRYYNEGSKKPILYINYVFSKAEEKFSMLE | | |
| | KLLTTLHKALIKAVDLAMGTEIMVYSPIVSMTK | | |
| | IQKTPLPERKALPVRWITWMTYLEDPRITFHYD | | |
| | KTLPELKDVPSVYQNDIPIVPHPSQYSMVFYTD | | |
| | GSAIKNPNPTKTHSAGMGVVQGKFNPEFQVVN | | |
| | QWSIPLGNHTAQLAEVAAVEFACKQALKITGPV | | |
| | LIITDSFYVAESANKELPYWKSNGFVNNKKKPL | | |
| | KHVSKWKSIADCLSLKTGITIKHEKGHQPSHTS | | |
| | VHTEGNALADKLATQGSYVVNNIIKPSLDAELD | | |
| | QVLQGNLP | | |
| 8166 | TIRVPIEEYKERIIQQSTLPRDYKDKLRTLLEKYN | YP_ | Central |
| | ILWQHWENQVGHRRIFPHNIATGTCKPKPQRQY | 009508546 | chimpanzee |
| | PINPKARASIQVVIDDLLKQGVLIKQTSVMNTPV | | simian foamy |
| | YPVPKPDGRWRMVLDYREVNKTIPLIGAQNQH | | virus |
| | SLGLLTTLVREKYKTTLDLANGFWAHPITPESY | | |
| | WITAFTWQGLQYCWTRLPQGFLNSPALFTADV | | |
| | VDLLKEIPNVQVYVDDLYISHEDPQEHLDVLDK | | |
| | IFQKLKDAGYVVSLKKSEIAQSTVEFLGFNITKE | | |
| | GRGLTESFKTKLLDLKPPETLKQLQSILGLLNFA | | |
| | RNFVSNFSELVKPLYQLISTAKGNNISWSNENTK | | |
| | QLQQLISALNNADNLEERKPDVKLIVKLNASPS | | |
| | AGYIRFYNETGKKPIMYINYVFTKAEIKFSPLEK | | |
| | LLVTLHKALIKALDIAMGKEILVYSPIVSMTKIQ | | |
| | KTPLPERKALPIRWITWMTYLEDPRISFYYDKTL | | |
| | PELKLVPEVQEKEKIIASRHPSQYTSVFYTDGSAI | | |
| | RSPDVSKAHSAGMGVVQGYFDPEFKISNSWSVP | | |
| | LGDHTAQYAEVCAVEFACKKALSVSGPVLIITD | | |
| | SFYVAESATKELPYWRSNGFLTNKKKPLKHVS | | |
| | KWKVIADCLQSKPDIVILHEKGHQPNNTSIHTEG | | |
| | NALADKLATQGSYTVNNIQNPSLDAELDQILQG | | |
| | NFP | | |
| 8167 | TVKLPVQDFKKELINKANINNEEKKQLAKLLDK | YP_ | Yellow-breasted |
| | YDILWQQWENQVGHRKIPPHNIATGTVAPRPQR | 009508582 | capuchin simian |
| | QYHINTKAKPSIQQVIDDLLKQGVLVKQTSVMN | | foamy virus |
| | TPVYPVPKPDGKWRMVLDYRAVNKTIPLIGAQ | | |
| | NQHSLGILTNLVRQKYKSTIDLSNGFWAHPITK | | |
| | DSQWITAFTWEGKQHVWTRLPQGFLNSPALFT | | |
| | ADVVDLLKDIPGISVYVDDIYFSTETVSEHLKIL | | |
| | EKVFKILLEAGYIVSLKKSALLRHEVTFLGFSITQ | | |
| | TGRGLTSEFKDKIQNITPPKTLKELQSILGLFNFA | | |
| | RNFVPNFSEIIKPLYSLISTAEGNNIKWTSEHTRH | | |
| | LEEIVSALNHAGNLEQRDDESPLVVKLNASPKT | | |
| | GYIRYYNKGGQKPIAYASHVFTNTESKFTPLEK | | |
| | LLVTMHKALIKAIDLALGQPIEVYSPIVSMQKLQ | | |
| | KTPLPERKALSTRWITWLSYLEDPRIIFHYDKTL | | |
| | PDLKNVPETITEKQPKILPIIEYAAVFYTDGSAIR | | |
| | SPDKNKSHSSGMGIVQAIFKPELTIEHQWTIPLG | | |
| | DHTAQYAEISAVEFACKKANNISGPVLIVTDSD | | |
| | YVARSVNEELPFWRSNGFVNNKKKPLKHISKW | | |
| | KNISDSLLLKRDITIVHEPGHQPSHTSIHTQGNNL | | |
| | ADKLATQGSYNVNSIVKNPSLDAELEQLINGHS | | |
| | M | | |
| 8168 | TIKLPVQDLKNTLVSQANIGKEDKIKLAKLLDK | YP_ | Spider monkey |
| | YDDLWQQWDNQVGNRKITPHNIATGTYPPKPQ | 009508561 | simian foamy |
| | KQYHINPKAKPSIQIVINDLLKQGVLRQSTSPMN | | virus |
| | TPVYPVPKPDGKWRMVLDYRAVNKTIPLIAAQ | | |
| | NQHSLGILTNLIRHKYKSTIDLSNGFWAHPITED | | |
| | SQWITAFTWEGKQHVWTRLPQGFLNSPALFTA | | |
| | DVVDILKEVPGVSVYVDDIYISSPTMEEHFQVL | | |
| | DSIFRKLLETGYIVSLKKSALARYEVNFLGFVISE | | |
| | TGRGLTSEFRERLQEITPPTTLKQLQSILGFLNFA | | |
| | RNFVPNFSELVQPLYQLISTASGNFIQWTAEHTL | | |
| | RLNELISALNHAGNLEQRRGDSPLVVKVNASDK | | |
| | TGYIRYYNDNSLIPIAYASHVFSTAELKFTPLEK | | |
| | LLVTMHRALLKGIDLALGQPIKVYSPIASMQKL | | |
| | QKTPIPERKALSTRWVTWLSYLEDPRITFYYDK | | |
| | TLPDLKHVPASTDNNIITLLPITEYEAVFYTDGS | | |
| | AIKSPKTEQTHSAGMGIVMVVYTPEPNITQQWS | | |
| | IPLGDHTAQYAEISAVEFACKKASLLQGPVLIVT | | |
| | DSDYVARSANKELPFWRSNGFLNNKKKPLKHIS | | |
| | KWKNISDSLLLKRNITIVHEPGHQPSKTSIHTLG | | |
| | NSLADKLAVQGSYSVNTINKIPSLDAELNQILEG | | |
| | NLP | | |
| 8169 | TIKLPVQEQKDSLVSQANIKKEDKIKLAKLLDK | YP_ | White-tufted-ear |
| | YDALWQNWENQVGNRKITPHNIATGTEPPRPQ | 009508577 | marmoset simian |
| | KQYHINPKAKPSIQIVINDLLKQGVLKQVTSPM | | foamy virus |
| | NTPVYPVPKPDGKWRMVLDYRAVNKTIPLIAA | | |
| | QNQHSLGILTNLVRHKYKSTIDLSNGFWAHPITS | | |
| | DSQWITAFTWEGKQHVWTRLPQGFLNSPALFT | | |
| | ADVVDILKEIPNVSVYVDDIYFSSPTVEEHLDTL | | |
| | EKIFDKLLQAGYIVSLKKSALARYEVNFLGFAIS | | |
| | ETGRGLTSEFKERLQEITPPTTIKQLQSIMGFLNF | | |
| | ARNFIPNFSELVQPLYQLIATASGNFIHWTTEHT | | |
| | LRLREIISALNHAGNLEQRVGDSPLVIKVNASDR | | |
| | TGYIRYYNDGSIVPIAYASHVFSSAEQKFTPTEK | | |
| | LLVTMHRALLKGLDLALGQPIRVYSPVASMQK | | |
| | LQKTPLPERKALSTRWVTWLSYLEDPRITFFYD | | |
| | KSLPDIKTFLPQLLLNAYMLPITQYEAVFYTDGS | | |
| | AIKAPKLTQAHSAGMGIVMVIFNPEPTVKQQWS | | |
| | IPLGDHTAQYAEISAVEFACKKASLLTGPVLIVT | | |
| | DSDYVARSANDELPFWRSNGFLNNKKKPLKHIS | | |
| | KWKNISDSLLLKRNITIVHEPGHQPSKTSIHTFG | | |
| | NSLADKLAVQGSYTVNTVHTPSLDAELNQILNK | | |
| | DFP | | |
| 8170 | TIKLDLEEQQGTLLNNSILSKKGKEELKQLFEKY | YP_ | Feline foamy |
| | SALWQSWENQVGHRRIRPHKIATGTVKPTPQK | 009513249 | virus |
| | QYHINPKAKPDIQIVINDLLKQGVLIQKESTMNT | | |
| | PVYPVPKPNGRWRMVLDYRAVNKVTPLIAVQN | | |
| | QHSYGILGSLFKGRYKTTIDLSNGFWAHPIVPED | | |
| | YWITAFTWQGKQYCWTVLPQGFLNSPGLFTGD | | |
| | VVDLLQGIPNVEVYVDDVYISHDSEKEHLEYLD | | |
| | ILFNRLKEAGYIISLKKSNIANSIVDFLGFQITNEG | | |
| | RGLTDTFKEKLENITAPTTLKQLQSILGLLNFAR | | |
| | NFIPDFTELIAPLYALIPKSTKNYVPWQIEHSTTL | | |
| | ETLITKLNGAEYLQGRKGDKTLIMKVNASYTTG | | |
| | YIRYYNEGEKKPISYVSIVFSKTELKFTELEKLLT | | |
| | TVHKGLLKALDLSMGQNIHVYSPIVSMQNIQKT | | |
| | PQTAKKALASRWLSWLSYLEDPRIRFFYDPQMP | | |
| | ALKDLPAVDTGKDNKKHPSNFQHIFYTDGSAIT | | |
| | SPTKEGHLNAGMGIVYFINKDGNLQKQQEWSIS | | |
| | LGNHTAQFAEIAAFEFALKKCLPLGGNILVVTD | | |
| | SNYVAKAYNEELDVWASNGFVNNRKKPLKHIS | | |
| | KWKSVADLKRLRPDVVVTHEPGHQKLDSSPHA | | |
| | YGNNLADQLATQASFKVHMTKNPKLDIEQIKAI | | |
| | QACQNNERLP | | |
| 8171 | TTLVPLQEYQERLLKQTALPNKEKTMLQSLFLR | YP_ | Guenon simian |
| | YDALWQHWENQVGHRRIKPHHIATGTVPPRPQ | 009666126 | foamy virus |
| | KQYPINPKAKPSIQVVINDLLKQGVLVQQNSTM | | |
| | NTPIYPVPKPDGKWRMVLDYREVNKTIPLIAAQ | | |
| | NQHSAGILSSIFRGKYKTTLDLSNGFWAHPITPE | | |
| | SYWLTAFTWQGQQYCWTRLPQGFLNSPALFTA | | |
| | DVVDLLKEIPNVQAYVDDIYISHDDPVEHVQQL | | |
| | EKVFSLLLNAGYVVSLKKSEIAKHEVEFLGFNIT | | |
| | KEGRGLTDTFKQKLLNITPPKDLKQLQSILGLLN | | |
| | FARNFIANFSELVRPLYNIVSSANGKYITWTQEN | | |
| | SQQLQNIISTLNSAKNLQERNPEVRLVMKVNTS | | |
| | PSAGYIRFYNEATKQPIMYLNYVYSKAETKFTM | | |
| | TEKLLTTIHKGLIKALDLAMGQEILVYSPIVSMT | | |
| | KIQKTPLPERKALPIRWITWMSYLEDPRIQFYYD | | |
| | KTLPELLQVPKVTEDEIAKTKHPSEFNMVFYTD | | |
| | GSAIKHPNIKKSHSAGMGIAQVQFKPDFTIVNT | | |
| | WSIPLGDHTAQMAEIAAVEFACKKALKITGPVL | | |
| | VVTDSFYVAESANKELPYWQSNGFVNNKKKPL | | |
| | KHVSKWKSIAECLQLKPDIVIMHEKGHQPSNTT | | |
| | FHTEGNNLADKLATQGSYVVNTNTTPSLDAEL | | |
| | DQLLQGHTP | | |
| 8172 | IAQKAINIFEKVQVFQRKIYLSTKADNKRKFGVL | WP_2022638 | Enterococcus |
| | YDKVYRKDILKVAWFYVKRNKGSAGIDDFTIEE | 42 | faecium |
| | JEAYGVQKFLDEIEDQLRNKKYQPKAVKRVYIP | | |
| | KANGKKRPLGIPTVRDRVVQTAVKIVIEPIFEAD | | |
| | FQEFSYGFRPKRSANQAIREIYKYLNYGCEWVI | | |
| | DADLKGYFDTIPHDKLLLLVKERVTDKSIIKLLS | | |
| | LWLEAGIMEDNQVRSNILGTPQGGVISPLLANIY | | |
| | LNALDRYWKNNRLEGRGHDAHLIRYADDFVIL | | |
| | CSNNPKKYYQYAKQRIDKLGLTLNEEKTRIVHA | | |
| | TEGFDFLGYTLRKSKSHKSGKYKTYYYPSRKS | | |
| | MKSIKGKVKDVIQTGQHLNLPDVMERLNPMLR | | |
| | GWANYFKAGNSKQHFKSIDNYVIYNLTIMLRK | | |
| | KHKKSGKGWREHPPSWYYNYFGLVCLRKLSTN | | |
| | INDDSQRYGR | | |
| 8173 | KKGKPIYVPNSFGEELGKKIKRKVAKKYTFDNF | A0A0H1A76 | Aquamicrobium |
| | IYHFKDGSHVVALHRHRKNAFFCRVDISRFFYS | 8 | sp.LC103 |
| | VKRNRLKRVLKSIGISKAEHYAKWSTVKNPFDG | | |
| | GGYVLPYGFIQSPILATLVLAESPIGAFLRGLPET | | |
| | ITPSVYMDDLCLSGQDEAELKVAFDGLVAAVV | | |
| | DAGFTLNDEKTREPAPQIDIFNCSLESGSTVVLP | | |
| | ERIEEFF | | |
| 8174 | FELKYRTRGKWIFVPTDQCERKGRRIIQYFSRFK | UPI000365A | Bradyrhizobium |
| | FPDYFYHYQPGGHIAALHAHLQHKLFFKIDIQN | 698 | sp.WSM2793 |
| | FYYSIARMRVTRALRSHAYPGANTLAKWSTVR | | |
| | NPYGGPLAHVLPIGFVQSPLLASLVLMKSPVAE | | |
| | AIERARKSGVTISVYMDDFIGSHDDEATLQAAY | | |
| | ADIRDASVRAGLLPNPAKLVAPTAAITAFNCDL | | |
| | SFGAANVSIDRVAKY | | |
| 8175 | TVKFETYRYSYLRKGKPVFVPSERGEAIGRELK | A0A2E4Z3C8 | Mesorhizobium |
| | AKVEAAINFEDIYYHLREGGHVAALHAHRDHR | | |
| | YFARVDIERFFYGISRNRVARELHGIGIEKAGYF | | |
| | AKWSCVKNPFEEPRYALPYGFVQSPILATLVLT | | |
| | RSGVGAFLRGLPENVTASVYMDDIALSCDDAE | | |
| | ALQATYAGLRAALEESNFAINEDKAQRPAEAIE | | |
| | LFNCALSQRFTSVLQARRDDFF | | |
| 8176 | LENYKEKYKHEGKFIFVPNYECIRKGLRIVEFCR | UPI000660A | Microvirga |
| | DRLEFPDCFYHYRNGGHVAALHRHLDNRFFFRI | 62D | massiliensis |
| | DLQNFYYAISRNRVCAALHASGFHKARTYGKW | | |
| | SCVRNPVAEDPRYSLPIGFVQSPALASLVLMRSP | | |
| | IMGAISAAERDSVFISVYLDDLIGSSTDFSLLERA | | |
| | YYTILEACGAANLQVNARKLLPPASEIHAFNCV | | |
| | LKHGLAEVTDERIRKFV | | |
| 8177 | TVAIRNYSFKYDRRGKPVFAPSNVGRRIGNEVK | A0A0Q6K2A | Sphingorhabdus |
| | EAVEGAFAFSPLYFHFRAGGHVAAIHHHRPHRF | 2 | pulchriflava |
| | FARIDISRFFYSISRRRVQSALDRIGVANAAFYA | | |
| | KWSTVTNPFEAPRYALPYGFVQSPILASLVLASS | | |
| | SVGEHLSSLSPDVTVSVYVDDISLSADRLDALQ | | |
| | AAYDATLAVLESDGFLVSADKLRPPAAAIDVEN | | |
| | CDLAQGLSKVQDDRINQFMADLPSHEAE | | |
| 8178 | PVQFQNFDYTYQRNGKPVFAPSPLGRQIGEDIK | UPI00068F6 | Sphingomonas |
| | EQVEAKYQFDDFVFHLRKKGGHVAALHSHRPH | D74 | sp.Leaf339 |
| | GYFARVDIRRFFYSVARNRVQRALATIGIPRAR | | |
| | HYAKWSCVKNPYDLPTYSLPYGFVQSPILASLV | | |
| | LMESAVGSFLRGLVAENHVTVSVYMDDISLSSD | | |
| | DLPRLQAAFNRLVHDLAEARFQVSPAKLRPPGP | | |
| | VMDLFNCDLRQGETVVREERIDLFEAEPQSH | | |
| 8179 | YDHHFALKPGTRVYIPTEMGRKRGTEIKGAIEG | UPI000C80A | Tsuneonella |
| | LWKPPANYFHLLEGGHVAAVKSHRNATWLAS | 9D7 | flava |
| | LDLQRFFDQITRTKIHRALKVIGLPHQDAWEMA | | |
| | CDSTVDKKPPKRHFSLPFGFVQSPIVASVVLAQS | | |
| | ALGGAIRNLVAGGLTVTVYVDDITISGSSEEQVF | | |
| | AAVEQLETGAELAGLAFNPEKTQLPNGAVTSFN | | |
| | IAFGSGALEVKEDRMAEFEVAIRNGNEYQIEGIL | | |
| | GYVGSVNHGQA | | |
| 8180 | KWLHKFERKPGRWVFEPSPEARAEGVEVKELV | IOHPP5 | Burkholderia |
| | ESHWKAPSYYFHLRQGGHVAALMRHQRSTCFV | | lessedis |
| | KVDIADFFGSVSRSRISRVLKEFVSHAEARRIAT | | |
| | ASTVQHPEDPARQILPYGFVQSPLLASLALHKSG | | |
| | LGKYLDQLHRENSVVVTVYVDDIIVSGNDPEEL | | |
| | GDVLTTMKTKAQRSRLAFSDDKEQGPAATISAF | | |
| | NIELAAGTPLSVLPPKLAEFREAFQASTSELQRA | | |
| | GIQGYVRSVNPVQA | | |
| 8181 | KRWEHRFQVKSGRWVFVPTPASRAVGEQIRAR | UPI000C157 | Stenotrophomonas |
| | VAKAWSPPAFYYHLRKGGHVAALRAHADSTY | 4FC | maltophilia |
| | FFRCDLKNFFGSINLSRVTRCLKRYFSYVEARG | | |
| | MASASVVIAPGATKTMLPYGFVQSPLLASLALD | | |
| | QSRAGAYLRKLSAQEGITVSVYMDDIVVSSLEI | | |
| | GLLDKIKAELGEKAEKAGISLNQEKTEGPSTLVT | | |
| | VFNVELSHNSLVISEERMAEFREALMIASCDAV | | |
| | AMGILGYVASVNDAQLSKLY | | |
| 8182 | RNFNHKIDLGNGKWAYVQEKHLIPHARRMTNL | A0A1V1UJF | Roseovarius |
| | HIERARFPKFYFHFHSGGHVMASKLHSENSFFSHI | 8 | sp. A-2 |
| | DLERFFYHVSKNKIVRSMKKVGFGFREAEGFAV | | |
| | VSTVRSEKGFTLPFGFVQSPALAALVLDQSQLG | | |
| | KTLREIAPNVQTTLYGDDILLSSKNEKTLREASD | | |
| | NVLLACQKANFPTNQEKTKVVQTKITAFNINISR | | |
| | NCLEITEERMNDFYVNIRHLGNCPTTQGILGYVS | | |
| | RVNQRQAE | | |
| 8183 | KWLSRFRLKSNTWVYVPTFETVKEGKLFKKAIE | UPI0009C18 | Pseudomonas |
| | FKWIPPTNYYHLRSGGHVEAVKYHLGGKFFVH | C3C | lurida |
| | ADISKFFNSINRSRITRELKPYFGYERSRAIAMES | | |
| | TVSIPVDSGQIFALPFGFVQSTIIASLCLRKSSLGK | | |
| | TIDVLNKTDGIRVSVYVDDIVVSTQCLEKAKAA | | |
| | FLMIQKSAERSGFLLNKEKSQGPSDKITAFNIDL | | |
| | RQNFMEVTSWRFSELLSSYKDATSDKKKSGIW | | |
| | GYVNSVNSAQASML | | |
| 8184 | WSNRFEIKPGRWVYNPTKESRLIGQKIIKLINKS | A0A1E5DOI7 | Vibrio genomo |
| | WKKPPYYYHLRCGGHVEALAIHLENQFFATVDI | | sp. F6 |
| | SDFFGHISRSRITRALKPIVGYEVARKIAKLSTIK | | |
| | TTENYTHSHHLPYGFNQSPILASICLFNSTLGKY | | |
| | LETAANDENITVSVYMDDIVISSQNEELLTQTFD | | |
| | QIFFTAKKSKFVINENKTKPVAKQTEAFNIVITH | | |
| | GDMKIEYDRLVKFQHAYSGSNSEHQRHGIGSY | | |
| | VGSVNKAQAKLL | | |
| 8185 | WKNRFEVKPGTWVYEPTLESKKYGRELITQIRK | UPI00073E7 | Vibrio |
| | KWKAPEYYYHLRDGGHVKALEKHTANNFFAS | 897 | parahaemolyticus |
| | LDIKDFFGSISRTRVTRTLKPLFGYDIARKLAKLS | | |
| | SVKNDGSKAHSHSIPYGYVQSPILASICLHKSTF | | |
| | GSELDSCFNDQNVTISVYVDDIVISSNDKKLLKH | | |
| | WCERLKNAAKRSKFTLNALKESPADSTVVAFNI | | |
| | EVTHNSMVITKERFKLLYEAYQNSQSIMQRKGI | | |
| | GGYVGTVNKSQARLLDL | | |
| 8186 | DKWIHKFEIKEGRWVYVPSEKTREIGGKIHFYIK | A0A502GKH | Ewingella |
| | HKWNYPLYMYHLRKGGHVAAANRHIKKQYFS | 5 | americana |
| | LIDISDFFGSTSQSRVTRELGKLIPYAKAREIAKL | | |
| | STVRSPPSNGLKHVIPYGYPQSPILATLCFHQSFC | | |
| | GKLINIISKSGQISVSIYMDDILLSSDDLSQLEIAF | | |
| | NSVKAALAKSGYCINERKTQSPSTMVKVENLEL | | |
| | SQQSLRVSPKRIVEFLQAYISSKNAAERKGIASY | | |
| | VGSVNKSQSTIFK | | |
| 8187 | KPTWLHKFEVKENRWVYIPDAETHHLGQKIHT | A0A6S5JQQ | Enterobacter |
| | YIKHKWKSPLYMFHLREGGHVAAANYHIKKKY | 2 | cloacae |
| | FSLIDISNFFGATSQSRVTRELGRLIPYVKAREIA | | |
| | RLSTVKNLNGNGLKHVIPYGYPQSPILASLCFHQ | | |
| | SFCGSTINTVSKSGHVSVSIFMDDILLSSDDLGQ | | |
| | LENAFDIILEAIRRSGYTVNENKTQPPSLMVNVF | | |
| | NLELSQNSLRVTSKRIVEFLQAFISSKNPHERKGI | | |
| | ASYVGSINTAQAKLFR | | |
| 8188 | ERWNSKFQIKKGAWVFIPTKETIDNGLQIKKLIE | A0A1H3PXH | Nitrosomonas |
| | KHWSFPKYYFHLIKGGHVRALHEHKKHKFFIHL | 4 | halophila |
| | DIKNFFGQINRSRVTRSLKEYVSYEKAREIAVES | | |
| | TVRLPESTEVKYILPFGFVQSPIIASICLSKSALGR | | |
| | HLTSLKQQNEYAVSIYMDDILVSANNSEKLMM | | |
| | EIMRIKAASEKSKLLLNAAKQVGPDTRIKAFNIE | | |
| | ISQDLLQITPSKLQELANTYATSENDHQRAGIFN | | |
| | YVLSVNPSQVEAF | | |
| 8189 | NTENEWLHKFEIKPGRWVYIPNEATLKQGKLIH | A0A7Y8YG | Kalamiella |
| | AYIRSKWKSPLYMFHLREGGHVAAANHHVQK | K8 | piersonii |
| | KYFALIDIQDFFGATSQSRITRALGNFIPYDKAR | | |
| | KIAKLSTVKNQNGNGLKHVIPYGYPQSPILASFC | | |
| | FHQSFCGDLIKNISKSGDISVSIYMDDILLSGNDL | | |
| | GRLEDTFKAVNEALVKSGYTVNRSKTQLPSTIV | | |
| | NVFNLELSQNSLRISAKRIVDFLQTYISSSHPPEK | | |
| | KGIASYVGSVNKEQARLFK | | |
| 8190 | TYEAKWINKFLLKPKKWVFVPSKQSKIIGKNIC | A0A259PL07 | Polynucleobacter |
| | RLLRKHWDPPEYFYHLRNGGHVEALRAHEQNQ | | sp.86C-FISCH |
| | YFSRLDIDNFFGSITRSRITRSLKKFVSYKTAWDI | | |
| | AHQSTVKDPDCISMRFMLPFGFIQSPLLASICLD | | |
| | QSFLGEFLKLLNKNPQLSLSVYMDDLVISSNDR | | |
| | DLLLSISADLKNAIVKSHWSSNEQKEDICKQLIM | | |
| | AFNIEISHQSTRISDPRMQEFKYSYKHAANSNSK | | |
| | LGIVNYIRSVNPVQG | | |
| 8191 | STPSDWLHKFEIKAERWVFVPSKETLKRGQKIH | A0A7T9UC3 | Serratia |
| | AYIKQKWKYPRYMFHLRNGGHVAAANFHLKS | 7 | plymuthica |
| | NYFSLIDVSDFYGTTSQSRVTRELGRLVPYVKA | | |
| | REIARLSTVVNPNRNGFKHVIPYGYPQSPILASL | | |
| | CFHHSFCGGVISSISKSERVFVSVYMDDILLSSD | | |
| | DMNLLVEAFDTVRQALRKSGYTLNENKTQPPS | | |
| | PKIQVFNLELGHNHLRVTPKRIVEFLKVFTSSSN | | |
| | EHERKGIASYVGSINKSQAKLFR | | |
| 8192 | EKWKHKFLLKENKWVHIPSEEMIKYGSALHRYI | A0A376EX1 | Cronobacter |
| | RKNWRFPLYYYHLRNGGHVAAARLHKRNNYF | 3 | universalis |
| | CLIDIKGFFESTSQSRVTRELKKIIPYDKARLIAK | | |
| | LSTVRLPNAVGHKFAVPYGYPQSPVLATLCLQN | | |
| | SYAGNVIDSFHRSGCVTVSVYMDDIILSCKSLVT | | |
| | LNQHFDVLCKALRKSRYELNASKTQSPAAKISV | | |
| | FNLELGHQHLKVESERMMLFIQAFAKSGNEHER | | |
| | KSIAKYVNTVNASQARHHFPK | | |
| 8193 | TIEVQRWEDKFEIKPGVWVYIPSVEARKVGGKI | A0A774SQ2 | Enterobacteriaceae |
| | LQAVRNKWIPPLYFYHLRTGGHLKAARLHLKS | 6 | |
| | DFFAVVDIKQFFQSTSRSRITRDLKSYFTYSQAR | | |
| | EISTFSTVRNLSHSPHKHVLPFGFVQSPILATLCL | | |
| | DKSYFGSLLRRLNKHHDLKLSVFMDDVIISSND | | |
| | LAQLQAAYDEALVAMRKSGYQANMSKTQAPS | | |
| | SKISVFNLTLSKGVMKVTSQKMSDFLIDFYSSN | | |
| | YEPHRIGVKNYVEAVNPGQAKLFKL | | |
| 8194 | TIEVQRWEDKFEIKPGVWVYVPSVEARKVGGKI | A0A5H7Z7D | Klebsiella |
| | LQDVRNKWIPPLYFYHLRTGGHLKAARLHLKS | 3 | pneumoniae |
| | DFFAVVDIKQFFQSTSRSRITRDLKSYFTYSQAR | | |
| | EISTFSTVRNLSHSPHKHVLPFGFVQSPILATLCL | | |
| | DKSYFGSLLRRLNKHHDLKLSVFMDDVIISSND | | |
| | LAQLQAAYDEALVAMRKSGYQANMSKTQAPS | | |
| | SKISVFNLTLSKGVMKVTSQKMSDFLIDFYSSN | | |
| | YEPHRIGVKNYVEAVNPGQAKLFKL | | |
| 8195 | TLELQRWKHKFEIKPGVWVFAPSPEAVKNGSKI | UPI000666D | Escherichia coli |
| | LNAVRKHWNPPLYFFHLRTGGHLKATRLHLKN | B4B | |
| | TYFAVVDIKQFFLSTSRSRVTRCLNEYFSYEKAR | | |
| | EISRYSTVKNPSSGLHKYVLPFGFVQSPILATLCL | | |
| | DKSYLGSLLRKLSRKGTMKLSIYMDDVIISSNDF | | |
| | QTLQTTYQEILQAMEKSGYKLNLKKSQPPSNKI | | |
| | VVFNISLRHGNMSVTAERLSEFLIDFYASNDEQ | | |
| | HKLGIANYVRSVNLEQAKLFR | | |
| 8196 | VKWEHRFELKKDKWVYVPTKEMRLYGTEIHN | A0A7Z8XG66 | Enterobacter |
| | HLRAKWIPPLYFYHLRDGGHVACAKKHLHNRY | | hormaeche |
| | FALIDIKNFFESTGQSRLTRELKTYLTYNNARQV | | |
| | AKLSTVRNPLPERPKYIIPYGYPQSPILSTFCLHK | | |
| | SFCGNLFKQLHVNPDIDISIYMDDIILSANDLSSC | | |
| | ESAYRLLSEGLERSGYQMNTLKSQFPSEKIHVF | | |
| | NLELKHNSLRVLPFRLIEFLIAYTKSKNRHERKG | | |
| | IASYVGSVNTDQAKLFK | | |
| 8197 | EIEVQRWEHKFEIRPGVWVYVPSVRTSELGERIL | A0A0V9E1K6 | Enterobacter |
| | QSIRNKWIPPLYFYHLRTGGHLKAARLHLKSSFI | | sp.50588862 |
| | AVIDIKHFFQSTSRSRITRDLKAYFTYSQAREIAK | | |
| | FSTVKNLSDTPHKHVLPFGFVQSPMLATFCLDK | | |
| | SHLGSLLRRLNKHPNVKLSVYMDDVIISSNDIV | | |
| | QLQTTYDEILLAMDKSGYQVNMIKTQAPSSLIK | | |
| | VFNLYLSKGNMKVTSQKMSDFLIDFYASDYEP | | |
| | HKIGVKNYVESVNPEQAKLLKL | | |
| 8198 | SNMAPRWINKFEIKPNVWVYEPSEACREEGAEI | UPI0005DB0 | Yersinia |
| | LRFINRKWKIPTYYYHLRRGGHVEALRVHIEND | 90F | enterocolitica |
| | WFVSLDIKEFFQSTSRSRVTRTLRNFLPYEKARII | | |
| | AKVSTVSNFTNDKFSHFIPFGYVQSPLLATICLH | | |
| 8199 | YSSFGQLIKELSRCEDVKLSVYMDDIILSSSSLEL | | |
| | LERTKTLLEESASRSHYKLNTLKVQGPAERITAF | | |
| | NIDLSHQSMVISPKRLLSFLVDFNSPDSDNLKRE | | |
| | GIRSYIYSVNSEQAEYF | | |
| | STIDVTLKEVADPIRLKLAWTKIKKKGSIGGVD | KPA10619.1 | Candidatus |
| | GVTISSFNANLEVNLSELSNQILTNQYTPEPLQA | | Magnetomorum |
| | AHIPKPGKSEKRQLGLPSLKDKIVQSSLASILSDF | | sp. HK-1 |
| | YEIHFSNCSYAYRPGKGSVKAIGRVRDFLNRKN | | |
| | YWIASVDIDNFFDSVDHEICTSILKEQISDQSIIRL | | |
| | ISLYFSSGMIKFDQWQDTEIGIPQGGAISPVISNIY | | |
| | LNKLDHFLHTLNAFFVRYADDIILFSNTQQSLSE | | |
| | TYQKTNEFLNKKLNLKLNALDNPIINVSKGFSFL | | |
| | GIYFHRCQLKIDFKRIDEKIEKMKYIIHKQKQID | | |
| | AVIKEINEFFNGVQRHYGNIIPDSYQLKNLESTV | | |
| | LDELSIFLAKMKNEGHINSKKACKLVLDPLVFM | | |
| | SERTKSQRDAVIDKIIADAFTIVDQKKDTDEKRI | | |
| | EKSVDSAIHQKRQAYAKKIATETE | | |
| 8200 | AGVQRTLPAETDGQDNGEASTFDLLERILSSNN | HHV47343 | Tissierellia |
| | MNAAYKQVVRNKGSHGIDGMKVDELLPHLKE | | bacterium |
| | NGNNLIEELKAGTYRPKPVRRVEIPKPDGGVRL | | |
| | LGIPTVVDRMIQQAIAQILTPIFDPEFSESSFGFRP | | |
| | GRSAHQAIKRAQEYMDEGYNWVVDIDLAKYF | | |
| | DTVNHDKLMALVARKVKDKRVLKLIREYLKA | | |
| | GVMINGVVIETEEGCPQGGPLSPLLSNIMLDELD | | |
| | KELEKRGHKFCRYADDCNIYVRSRKAAERTMQ | | |
| | SVTKFLEGKLKLKVNREKSAVDRPWKLKFLGF | | |
| | SFYRGKEGIRIRVHRKSIERVKEKIRNITSRSNGM | | |
| | SMDTRLLKLKQLIRGWVNYFRIADMKSLAQSL | | |
| | DEWTRRRLRMCIWKQWKRVRTRFQNLMKLGL | | |
| | DRQKALEFANTRKGYWRIANSPILSVTITNERLQ | | |
| | KRGYTGFVAELA | | |
| 8201 | LEQILARENLMTALHRVERNKGSHGVDGMPVQ | WP_080874617 | Oceanobacillus |
| | NLRAHIMEHWASIREQLETGTYYPQPVRRYEIH | | timonensis |
| | KEGGGMRKLGIPTVLDRFIQQAIAQVLTTIYDPT | | |
| | FSENSYGFRPKRRGHDAVRKARAYMKDGYRW | | |
| | VIDMDLEKFFDKVNHDRLMRTLSRRVKDPKVL | | |
| | QLIRRFLQAGIMEDGVVHPNTEGASQGGPLSPL | | |
| | LSNIVLDELDKELEKRGLHFVRYADDFHIYVRS | | |
| | KRAGHRIMESITNFIEKKMKLEVNKEKSAVDRP | | |
| | WKRKFLGFSFTFHKENPKIRIAKESIKRFKRRIRE | | |
| | LTSRKKSMNMGDRIEKLNQYLAGWLGYYQLA | | |
| | ETPTIFKELDGWIRRRLRMIRWKEWKKVKTKH | | |
| | KNLVKQGIKKGKAWEWANTRKSYWRTANSPIL | | |
| | HRALGDQYWSEQGLKSLTNSYLTKRWT | | |
| 8202 | LEQLLSRENLLQALKRVESNKGSHGVDGMTVK | WP_154118777 | Paenibacillus sp. |
| | SLREHIVQNWQKIRQAIEEGTYEPSPVRRVEIPK | | LC-T2 |
| | PNGGGVRKLGIPTVTDRMLQQAIAQVLTPWFDP | | |
| | PFSEHSYGFRPKRRGHDAVRKARTFMKEGYRF | | |
| | VVDLDLEKFFDRVNHDRLMMKIAEKVKDKKV | | |
| | LLLIRKYLQSGVMENGLVQPTREGAPQGGPLSP | | |
| | LLSNIVLDELDKELEKRGHRFVRYADDCNIYVK | | |
| | TLRAGERVKASVTRFIETRLKLKVNQAKSAVDH | | |
| | PWKRKFLGFSFSTDIEPKVRIAKQSLQKAKVRIR | | |
| | EITSRKKPMRMEERMKELNQYLMGWCGYFSLA | | |
| | DTPSIFQRMDAWIRRRLRMCLWKQWKNPRTKV | | |
| | KRLLSLGMPKGKAYEWGNTRKGYWRIAGSPIL | | |
| | SRALNNQYWESNGLKSLLDRYNSIRNIS | | |
| 8203 | LMKPILSRENLLNALKRVERNGGSYGVDKMST | WP_179156869 | Bacillus sp. |
| | QNLRLYIVEHWAELRNALQQGTYEPQPVRRVEI | | EB106-08-02- |
| | PKSNGGVRLLGIPTVLDRFIQQAITQTLTPIYDPT | | XG196 |
| | FSENSYGFRPQRRGHDAVRKAKGYIEEGYRWV | | |
| | VDIDLEKFFDKVNHDKLMGLLSKRIDDKTLLGL | | |
| | IRKFLNAGIMIGGVVSQNTEETPQGGPLSPLLSNI | | |
| | ILDVLDKELEDRGHKFVRYADDCNIYVKSKKA | | |
| | GIRTMEGITAFIEKGLSLKVNHDKSAVDRPWNR | | |
| | KFLGFSFTNRKEPKIRIAKQSIKRFKLKVKEITSR | | |
| | KSPIPMEIRIQKLNQYLVGWCGYYALADTPSVF | | |
| | KDLEGWIRRRLRLCYWKQWKLPKTRIRKLIGFG | | |
| | IDKHKAYEWGNTRKGYWRITNSPILSRALNNAF | | |
| | WRKEGLKSLYERYESLRHT | | |
| 8204 | LMERILSKENLLSALKRVERNKGSHGVDEMRV | WP_212605652 | Sporosarcina sp. |
| | QNLRTHIVNHWEPIKMELLKGDYEPQPVRRVEI | | Marseille-Q4063 |
| | PKPDGGVRLLGIPTVMDRFIQQAIAQILTSVYDP | | |
| | MFSDHSYGFRPKRSAHDAVRKAKGYLTEGNR | | |
| | WVVDIDLEKFFDKVNHDRLMGTLAKRIQDKRL | | |
| | LKLIRKYLKSGIMINGIVSASEEGTPQGGPLSPLL | | |
| | SNIVLDELDSELEKRGHKFVRYADDCNIYLKTK | | |
| | KAGSRVMNSVTSFIEKKLKLKVNLDKSAVDRP | | |
| | WKRKFLGFSFTFHKEPKVRIAKESLQRMKNKIR | | |
| | EITSRKKPCPLAYRIKKLNQYLMGWCGYFALA | | |
| | DTPSVFRNFDSWIRRRLRMCMWKAWKLPKTK | | |
| | VRKLTGLGIPKGKAYEWGNTRKSYWRISNSPIL | | |
| | HRALDNSYWNHQGLKSLSSRYEVLRNQP | | |
| 8205 | ERILSRGNLLSALKRVERNKGSHGVDGMSVQN | WP_126433867 | Bacillus |
| | LRRHIMEHWESLKAELLEGTYQPQPVRRVEIPK | | freudenreichii |
| | PDGGVRLLGIPTVTDRFIQQAIAQVLSSIYEPTFS | | |
| | NHSYGFRPNRSAHDAVRKTKEFIKEGKRWVVD | | |
| | IDLEKFFDRVNHDRLMGTLSKRIKDKRLLKLIRS | | |
| | YLKAGVMINGLVSANEEGTPQGGPLSPLLSNIV | | |
| | LDELDKELEKRGHAFVRYADDCNIYVNTQKAG | | |
| | SRVMASLTSFIEGKLKLKVNQGKSAVDRPWKR | | |
| | KFLGFSFTSGKEPKVRIAKESIKRMKQKIRDITSR | | |
| | KKPYPMEYRIEKLNQYLMGWCGYFALADTPSIF | | |
| | IRLDSWIKRRLRMCRWKEWKQPKTKMRKLIGL | | |
| | GVPKWQAYEWGNSRKGYWRISKSPILHKTLGN | | |
| | SYWSTQGLKSLISRYESLRHIS | | |
| 8206 | LLNQILSRENMLQALKRVEQNKGSHGVDWMP | WP_061797426 | Niallia circulans |
| | VQILRQHIVENWHSIREAIFKGTYEPMPVRRVEI | | |
| | PKSDGGVRLLGIPTVKDRLIQQAIAQVLSKIYDP | | |
| | MFSEHSYGFRPNRSAHDAVRKAKGYIKEGYRW | | |
| | VVDMDLEKFFDKVNHDRLMGTLAKRINDKPLL | | |
| | KLIRKYLQAGVMMDGVISSTEKGTPQGGPLSPL | | |
| | LSNIVLDELDKELESRGHKFVRYADDCNVYVKS | | |
| | KRAGERTRASIQRFIEKKLRLKVNEKKSAVDRP | | |
| | WKRKFLGFSFTSSKEPKIRIAKESLKRMKMKIRE | | |
| | ITSRKMPYSMRYRMEKLNQYLMGWCGYFALA | | |
| | DTKSLFIKLDGWIRRRLRMCQWKDWKKPKTRI | | |
| | RNLIHLGVPKGKAYEWGNSRKGYWRVSNSPIL | | |
| | DKTLDISYWNNQGFKSLQTRYKFLRHLS | | |
| 8207 | LMNQILSRENLLLALKRVERNKGSHGVDKMPV | WP_ | Lysinibacillus |
| | KFLRQHVVENWLTIKKQILEGTYQPQPVRRIEIP | 053592381 | |
| | KPDGGVRLLGIPTVTDRLIQQAIAQVLSNLYDPN | | |
| | FSNHSYGFRPKRSAHDAIREAKGYIKEGYRWVV | | |
| | DMDLEKFFDKVNHDRLMSTLAKKISDKPLLKLI | | |
| | RRYLQSGVMINGVVYDTDEGTPQGGPLSPLLSN | | |
| | IVLDELDKELEKRGHKFVRYADDCNIYVKTKR | | |
| | AGERVMASIKTFIEKTLRLKINEKKSAVARPWQ | | |
| | RKFLGFSFTSRKEPQVRIAKESIKRMKNKIRELT | | |
| | ARKKPFPMEYRIQQLNQYLIGWCGYFALADTK | | |
| | SIFESLDGWIRRRLRMCLWKDWKKPRTKVRNLI | | |
| | RLGVPDWKAYEWGNTRKSYWRISKSPILHRTL | | |
| | GNSYWSNQGLKSLQARYEILRYSS | | |
| 8208 | LLNQILSRENLLQALRRVEKNKGSHGVDKMPV | WP_ | Psychrobacillus |
| | QTLRQHMKDNWLSIKEQLLEGTYEPQPVRRIEIP | 142642771 | vulpis |
| | KPDGGVRLLGIPTVTDRLIQQAIAQVLSRLYDPT | | |
| | FSEHSYGFRPNRSAHDAVRKAKGYIKEGYRWII | | |
| | DMDLEKFFDKVNHDRLTSTLAKRINDKPLLKLI | | |
| | RKYLQSGVLINGIVLDINEGTPQGGPLSPLLSNIV | | |
| | LDELDKELEQRGHRFVRYADDCNIYVKSKRSG | | |
| | ERVMESVQTFIERKLRLKVNKKKSAVDRPWKR | | |
| | KFLGFSYTSNKEPKVRIAKESLQRMKKKIREITS | | |
| | RKKPYPMEYRIEQLNRYLIGWCGYFALADTKLI | | |
| | FGEIDGWIRRRLRMCLWKNWKKPRTKVRNLIR | | |
| | LGIPDGKAFEWGNTRKGYWRISNSPILSRALNN | | |
| | SYWSNQGFKSLQARYEILRYSS | | |
| 8209 | ERILSRENLLNAIKRVEKNKGKHGVDEMPVAAL | WP_ | Bacillaceae |
| | RGHIMLNWNELRKSLSEGTYIPSPVRRVEIPKPD | 061794427 | |
| | GKGKRKLGIPTVTDRFIQQAITQVLTKMYDPGF | | |
| | SECSFGFRPKRRAHQAVKLAQSYIEEGYRWVV | | |
| | DIDLEKFFDKVNHDKLMSKLAERINDRTLLKLI | | |
| | RRFLTSGVMEGGLVSPNLEGTPQGGPLSPLLSNI | | |
| | VLDELDTELERRGHRFVRYADDCNIYVKSKRA | | |
| | GERVMKAMTHFIEGKLKLKVNRDKSAVDRPW | | |
| | RRKFLGFSFTSNLKPKVRISPQSIKRFKDKIRKLT | | |
| | SRRRSIAMEVRIHDLNEYLVGWVNYYHLADTR | | |
| | SVITKLEGWVNRRLRMIRWKEWKLPRTKIKKLI | | |
| | ELGVPEGKAYKWGNTRKAYWRISKSPILHKTL | | |
| | GKAYWLSLGLKSISARYDLQRST | | |
| 8210 | DLMEQVVARENMWAALRRVEQNRGAPGVDG | EKP93788 | Thermaerobacter |
| | MTVEQLRGFLREQWSQVRAQLLAGTYKPQPVR | | subterraneus |
| | RVEIPKPGGGTRLLGIPTVLDRLIQQALLQVLTPI | | DSM 13965 |
| | FDPDFSEHSYGFRPGRSAHQAVEAARRHVEEGY | | |
| | AWVVDLDLEQFFDRVNHDVLMARVMRKVAD | | |
| | KRVRMLIRRYLQAGVMVGGVKVRTEEGTPQG | | |
| | GPLSPLLANILLDELDKELERRGHRFVRYADDC | | |
| | NIYVRSERAGHRVMAGVRRFLEKRLRLKINEQK | | |
| | SAVDRPWRRKFLGFSMYRGREGIRLRVAPQTV | | |
| | QRLKDRIRGLTSRTWPVSMPERIRRINAYLRGW | | |
| | LAYFRIADMAVLLRNVEGWLRRRLQACLWKQ | | |
| | WKRPRTRLRELRALGHPEWRVRQWALSRRGY | | |
| | WAMAGGPLNSALGKPYWLAQGLLSLTRCYHE | | |
| | LRRA | | |
| 8211 | ALLETILSRNNLITALKRVEANKGAPGIDGVPTE | WP_ | Caenibacillus |
| | QLRDDIRKHWKSIKRQLLEGTYKPAPVRRVEIP | 077616959 | caldisaponilyticus |
| | KPNGGVRLLGIPTVMDRFIQQAILQVLTPIFDPH | | |
| | FSPYSYGFRPKRRAHDAVRQAQKYIQEGYRYV | | |
| | VDIDLEKFFDRVNHDILMSRVARKVEDKRVLKL | | |
| | IRAYLKAGVMLEGVRVRSEEGTPQGGPLSPLLA | | |
| | NILLDDLDKELEKRGLKFCRYADDCNIYVRSPR | | |
| | AGQRVKQSVQKYLEKKLKLKVNEEKSAVDRP | | |
| | WKRKFLGFSFTSQREARIRLAPKSVQRFKNKIR | | |
| | QLTNPNWSLPMEERIRKLNQYTMGWMGYFALI | | |
| | ETPSPLKRLEEWIRRRLRLCRWHQWKRVRTRIR | | |
| | ELRALGLKEHEVFEIANTRKGAWRTTRTPQLHK | | |
| | ALGKAYWLKQGLKSLTQRYFELRQDWRTA | | |
| 8212 | ALLERILARDNLITALKRVEANRGAPGIDGVSTD | WP_ | Caldibacillus |
| | QLRDYIRTHWSSIRAQLLEGTYRPTPVRRVEIPK | 020154220 | debilis |
| | PNGGIRLLGIPTVMDRLIQQAILQELTPIFDPDFS | | |
| | PYSFGFRPGRSAHDAVRQAQRYIREGYRYVVDI | | |
| | DLEKFFDRVNHDILMSRVARKVKDKRVLKLIR | | |
| | AYLQAGVMIGGVKVQTEEGTPQGGPLSPLLANI | | |
| | LLDDLDKELEKRGLKFCRYADDCNIYVKSLRA | | |
| | GLRVKQGIQRFLEKKLKLKVNEEKSAVDRPWK | | |
| | RTFLGFSFTPEREARIRLAPKSIQRFKQRIRQSTN | | |
| | PNWSLPMEERIRRVNQYTMGWMGYFQLIETPSI | | |
| | LRNMEGWVRRRLRLCLWLQWKRVRTRMRELR | | |
| | ALGLNERTVLEIANTRKGAWRTTKTPQLHQAL | | |
| | GKSYWKAQGLKSLTQRYFELRQG | | |
| 8213 | CRKQNSERNSFGKVGVKPRGYRRGQSIDRQDLS | WP_ | Brevibacillus |
| | LVLRREKYRVELLEQILERKNLLEALKKVESNG | 198827538 | composti |
| | GAAGIDGVSTEHLRAYVVEHWEKIRQQLLDGT | | |
| | YKPAPVRRVEIPKPDGGVRLLGIPTALDRMLQQ | | |
| | AILQVLTPIFDPGFSPNSFGFRPGKRGHDAVRQA | | |
| | QRFIREGYRIVVDIDLEKFFDRVNHDILMSRVAR | | |
| | KVKDKRVLKLIRKYLKSGVMAGGIVSHTEEGTP | | |
| | QGGPLSPLLSNIMLDDLDKELERRGLHFSRFAD | | |
| | DCNIYVKTKRAGERVKASIERYLEGKLKLKVN | | |
| | KEKSAVERPWKRKFLGFSFTAQKEARIRISPKSL | | |
| | KRVKDKIRTLTKPTWSISMKERIQQLNQYLMG | | |
| | WIGYYALIETPKPLAELESWLKRRLRLCLWHQ | | |
| | WKRVRTRYRELRKLGLTHQQAFEIASTRKGAW | | |
| | RTSITPHLHKALGNAYWQSQGLKSVTQRYFEIR | | |
| | QGWRTA | | |
| 8214 | DLMEQILSRQNLLEALHRVESNKGAVGIDGVST | WP_ | Thermicanus |
| | EQLREYVMKHWGTIRQQLLEGTYKPSPVRRVEI | 039944322 | aegyptius |
| | PKPDGGVRLLGIPTVIDRLIQQAILQVLTPIVDPG | | |
| | FSPNSFGFRPNRRGHSAVRQAQRFIREGYRIVVD | | |
| | IDLAQFFDRVNHDILMSRVARKIKDKRVLKLIR | | |
| | AYLQSGVMTGGVCVSSEEGTPQGGPLSPLLGNI | | |
| | LLDDLDKELERRGLRFCRYADDCNIYVKTRRA | | |
| | GERIKASVTRFLEGRLKIKVNEEKSAVDWPWKR | | |
| | KFLGFSFTFEKEARIRLAPKSLKRVKNKIRELTTP | | |
| | TWSISMKERIRILNQYLMGWMGYYALIETPSIL | | |
| | KTLEQWTRRRLRLCLWHQWKRVRTRIRELRAL | | |
| | GLPERQVLEIANTRKGAWRTSQTPHLHKALGIA | | |
| | YWQSQGLKSLTQRYNELRQGWRTA | | |
| 8215 | RSRDGHRQQNTSQEGCQREVAVKPQGTVGVPS | WP_ | Thermobacillus |
| | PLPAQIAPSSRKAQDDLLEKMLERENLLKAYRK | 015253141 | composti |
| | VVQNGGAPGVDGVTVTELQAYLNTHWEAVKA | | |
| | ALLAGTYSPLPVRRVEIPKPGGGVRLLGIPTVM | | |
| | DRLLQQALLQVMEPIFDPHFSWHSYGFRPGKRA | | |
| | HDAVRQAQQYIQSGLRWVVDMDLEKFFDRVN | | |
| | HDILMARVARRIDDKRVLKLIRAYLNAGVMAG | | |
| | GVVVRTEEGTPQGGPLSPLLANILLDDLDKELT | | |
| | RRGLHFVRYADDCNIFVASKRAGERVMESVIRF | | |
| | VEGKLKLKVNRDKSAVDRPWNRKFLGFSFLSN | | |
| | KQATVRLAPKTIQRFKKKVREITDRSRPLTMEE | | |
| | RIHRLNRFMMGWIGYFRLAAAKNHCGNLDAW | | |
| | MRRRLRMCLWKQWKRPRTRLRNLRALGVPEW | | |
| | AARMMANSRRGPWEMSRNTNNALPTSYWEAK | | |
| | GLKSLLSRYLELC | | |
| 8216 | RSHEEQRQPNISQESCQQREAVKPSGYAGAPSSS | WP_ | Paenibacillus sp. |
| | SAQVAPSSREDQNNLLERLLEGDNLRLAYKRV | 083612306 | P32E |
| | VQNGGAPGVDHVTVANLQAYLKTHWETVKAE | | |
| | LLTGTYRPAPVKRVEIPKPGGGVRLLGIPTVMD | | |
| | RFLQQALLQVMNPIFDAQFSWYSYGFRPGKSA | | |
| | HGAVKQAQRYIQSGLRWVVDLDMEKFFDQVN | | |
| | HDMLMARVARKVADKRVLTLIRAYLNAGVMV | | |
| | DGKLERSWEGTPQGGPLSPLLANILLDDLDKEL | | |
| | TGRGLRFVRYADDCNIFVASKRAGERVKESVC | | |
| | RFVEGKLKLKVNREKSAVARPWHRKFLGFSFLS | | |
| | QKQATIRLAPKTISRFKEKIRELTNRTWSISMEE | | |
| | RISRLNRYMMGWIGYFRLASAKTHLQNLDQWI | | |
| | RRRLRMCLWKQWKRVRTRIRELRALGVPEWA | | |
| | CFMMGNSRRGVWEMSRNINNALRASYWEAKG | | |
| | LKSLLSRYLELG | | |
| 8217 | ERVLSQQNMHEALKQVRRNKGAAGIDGMETA | NCB17444 | Synergistales |
| | DLRPWLIEHWVRIREELLGGTYKPLPVRRVEIPK | | bacterium |
| | PDGGVRLLGIPTVVDRLIQQALHQELYHIFDPGF | | |
| | SESSYGFRKYHSARQAVEKARRYIGEGFRYVVD | | |
| | MDLEKFFDRVNHDMLMARVARKVTDKRVLKL | | |
| | IRAYLEAGVMTGGLFGETREGTPQGGPLSPLLA | | |
| | NIMLDDLDKELEKRGHRFVRYADDCNIYVRSR | | |
| | RAGERVMDGMRKFIENRLKLKVNEAKSAVDRP | | |
| | QNRKFLGFSFTGEKEPRIRIAPKALERFKNTVRR | | |
| | LTDRGRSTSTEERIRRLSEYLRGWAGYFRLAQT | | |
| | PSVFQKLDRWIRRRLRMCILKQWKNIRTKRRKL | | |
| | VSLGLSHDDAMKIASSRKGYWRLAETPQLHIA | | |
| | MGNRYFKTLGLVSLASG | | |
| 8218 | ASSRREQRQQKIPSGSYPQKEAVNPQGAGEAPS | NSW83172 | Syntrophothermus |
| | SLPAQTTGTTREANRTNLMEMVVERENMIRAL | | sp. |
| | KRVEANKGAAGVDGMKVEDLREYLKESWPEIR | | |
| | EQLLAGTYHPKPVRRVEIPKPDGGVRLLGIPTAL | | |
| | DRIIQQALLQILTPTFDPEFSPFSYGFRPYRKAEN | | |
| | AVRRAQEYISEGYRWVVDMDLEKFFDRVNHDI | | |
| | LMSRVARKVKDKRVLRLIRRYLQAGVMVNGC | | |
| | CVATEEGTPQGGPLSPLLANIMLDDLDRELMRR | | |
| | GHCFVRYADDCNIYVKSQRAGERVMESVKRFV | | |
| | EGELKLKVNLQKSAVDRPWKRKILGFSFTWDK | | |
| | EPRIRLAPKTIKRFKDKIRELTKRSRSQSMEDRIG | | |
| | ALNTYLMGWIGYFKLADTRSVLQSLDEWVRRR | | |
| | LRMCYLKQWKKPKTKLRNLIVLGIPADWAALIS | | |
| | GSRKGYWRLANTPQMNKALGLAFWRNQGLRS | | |
| | LVGRYDQLRFTS | | |
| 8219 | EEILADENLQEALQRVCANKGAAGIDGITTTEF | MBK8399227 | Leptospiraceae |
| | HKQMSEEWKETKQRLLLGKYKPKGVRRVEIPK | | bacterium |
| | PAGGIRMLGIPTVMDRFIQQAMLQRLTPIFDPEF | | |
| | SKFSYGFRPNKSAHDAVRQAKKYIEEGHKFVV | | |
| | DIDLEKFFDKVNHDILMHLVGKKIRDKRVLRLI | | |
| | GSYLRAGVMTNGVCIPNEEGTPQGGVISPILANI | | |
| | MLNELDKELEARGHKFCRYADDCNIYVKSMKA | | |
| | GERVKASITRFLNKKLKLKVNETKSAVDKPMN | | |
| | RKFLGFTFGNVDSVVIQISSQSLERVKNKIRELT | | |
| | NPMRSVSMEERIKVINRYIIGWLGYYSLIEVPETI | | |
| | ESIDGWLRRRMRSCQWQQWKKPKTRIRELIKL | | |
| | GLKESTARKMGYSRKGNWRCSRTPAMHKAMG | | |
| | IKHWKDRGLINLVARYEIYRESWRTA | | |
| 8172 | IAQKAINIFEKVQVFQRKIYLSTKADNKRKFGVL | WP_ | Streptococcus |
| | YDKVYRKDILKVAWFYVKRNKGSAGIDDFTIEE | 000561483.1 | |
| | IEAYGVQKFLDEIEDQLRNKKYQPKAVKRVYIP | | |
| | KANGKKRPLGIPTVRDRVVQTAVKIVIEPIFEAD | | |
| | FQEFSYGFRPKRSANQAIREIYKYLNYGCEWVI | | |
| | DADLKGYFDTIPHDKLLLLVKERVTDKSIIKLLS | | |
| | LWLEAGIMEDNQVRSNILGTPQGGVISPLLANIY | | |
| | LNALDRYWKNNRLEGRGHDAHLIRYADDFVIL | | |
| | CSNNPKKYYQYAKQRIDKLGLTLNEEKTRIVHA | | |
| | TEGFDFLGYTLRKSKSHKSGKYKTYYYPSRKS | | |
| | MKSIKGKVKDVIQTGQHLNLPDVMERLNPMLR | | |
| | GWANYFKAGNSKQHFKSIDNYVIYNLTIMLRK | | |
| | KHKKSGKGWREHPPSWYYNYFGLVCLRKLSTN | | |
| | INDDSQRYGR | | |
| 8220 | GTANELPLLEQALSDDRLLAGWERVRANAGGP | WP_ | Tibeticola |
| | GVDGVTVEQFGGKVLRALAGLRQRVTASHYQ | 124224144.1 | sediminis |
| | ALPLRRIEITRPGKAPRVLAVPCVADRVVQSAV | | |
| | ALTISPRLDPGFEDFSFGYRPGRSVPRAVQHLAE | | |
| | ARDSGLVWVAEADIQSCFDRIPWAALLQRLGE | | |
| | VLPDAGLLALIQHWLSLPLQWPDGHQQVRCMG | | |
| | VPQGSPLSPLLSNVFLDGMDKELAAGPWRVIRY | | |
| | ADDFVIAAASREEARRGLAQAARWLRRLGLRL | | |
| | NLDKTRVIHFDQGFSFLGVRFRGRQMSAVQPG | | |
| | AEPWVLPRATQPRPHSPSSKPAQHSRSPAPTAR | | |
| | ASAPATPPSAQPEPLGPAAPSPNAAASAQPSQPR | | |
| | AADATLQDLQRLSVAPPNEPSPPRLRT | | |
| 8221 | STLPTPSSTDQDSPPPFWTLARLAEALEHVSARQ | KFB76584.1 | Candidatus |
| | GGAGADEQTLAEFAADAEAQLGLLALQLTQGS | | Accumulibacter |
| | YRPAPARLIPVAKPGGGVRELLLPAVRDRIVQS | | sp. SK-02 |
| | ALARYLADLLEPDFGEASHAYRPGHSVATALH | | |
| | RLQALRDGGLVFVAVCDIHHFFDSVDHRRLFSL | | |
| | LDDLPLERRLREQMKTCVRIEVADVQGQGAWS | | |
| | LARGLAQGSPLSPVLANLFLMAFDAACARAGL | | |
| | ALVRYADDCVLACASETEAQSALAFAADALEN | | |
| | IGLALNTRKSRLASFAEGFEFLGAFCGAEGMLG | | |
| | GRPGEAACLPPTTGPVHEAAAADDERPPSHGHR | | |
| | PRLR | | |
| 8222 | NPTSDILERIAESSKSHTDGVFTRLYRYLLREDIY | WP_ | Butyricicoccus |
| | FTAYKNLYANAGASTKGTDDDTADGFGAKYV | 087017951.1 | porcorum |
| | SDIIESLRNLEYSPKPVRRIYIPKHNGKLRPLGIPS | | |
| | FRDKLIQDAIRQILEAIYEPIFSDDSHGFRPGRSC | | |
| | HTAFDRIKYGFNGTKWFIEGDIKGCFDNIDHKV | | |
| | LLNILSKKVKDSKLINLIGAFLKAGYMEEWKYF | | |
| | QTYSGTPQGGILSPILANIYLHELDKKVAEIKQR | | |
| | FDSNEPKKYTEEYGGICHRISTLHRKSKNNPDSP | | |
| | DREKWIAEEVELKRQRVKIPVYQDNNKRICYVR | | |
| | YADDFLIGVVGSKEDCVEIKAELKDFLAAELKL | | |
| | ELSDEKTKITHSSESARFLGYDVSVRRSQELKRR | | |
| | SDGVKQRTLNGTVMLNAPLKDKIEEFLISNGYG | | |
| | VRTADGRIVPIATKGLRNRSDFEIVSTYNSQMR | | |
| | GICNYYRMASNFPKLDYFVYIMEYSCLKTLASK | | |
| | HQTTMAKARGDRRTGKRWGVPYETKTGTKTLI | | |
| | FLNMTDIRKSRKAKLDNVDAVPKSVSKQNEIKN | | |
| | RLNAGICELCGCDSEPVVVHHVQSLKALKGKS | | |
| | AWERKMRSIRRKTLIVCETCHNKIHNKTFC | | |
| 8223 | KPTSEILERMYRNSEEHSDGIYTRLYRYLLREDI | CDB92781.1 | Acidaminococcus |
| | YMTAYKNLYANKGAGTEGVDNDTADGFGKEY | | intestini |
| | VNQIIDELKNQTYEPKAVKRVYIPKRNGKMRPL | | CAG:325 |
| | GIPSFRDKLIQDAIRQILEVIYEPVFSTHSHGFRPN | | |
| | RSCHSALKEISRSFRSTKWFVEGDIKGCFDNIDH | | |
| | TVLLNLLSEKIKDSKFINLIGKFLKAGYMDNWE | | |
| | YHKTYSGTPQGGILSPILANIYLHELDKKVEAM | | |
| | QKEFNAPADYAYTPAYGKKVRGIVKLQKRYGE | | |
| | CVDEAEKKELLKQIHKLEVEKRRLPYKDASDK | | |
| | KIAYVRYADDFIIGVSGSREDAERIKQELTLFVA | | |
| | TRLKLELSDEKTKITHSSGNAHFLGYDINVRRC | | |
| | QESKRKTNGVLQRTLNNSVELLIPMERIEKFMY | | |
| | DREIVIQGKDGKLIPWQRNSMAGLTDLEIVDTY | | |
| | NSQTRGICNYYCIASNFSKLTYFVYLMEYSCLK | | |
| | TLAKKHKTRISGIKRIFKCGKSWGIPYKTKKEKK | | |
| | RMMIVKFSDFKRGTVFDEPSIDTVKNHIHFNTR | | |
| | NSLEARLKACKCELCGAEGDGIAFEIHHINKMK | | |
| | NLKGKEQWEMAMIARKRKTLVVCKECHKKIH | | |
| | HSS | | |
| 8224 | KPTSEILERMYQNSAKHTNGVYTRLYRYLLRED | WP_ | Anaeromusa |
| | IYLTAYKKLYANKGAGTKGVDNDTADGFGME | 018704816.1 | acidaminophila |
| | YVHQIIDELKNQTYMPKPVRRTHIPKQNGKMRP | | |
| | LGIPSFRDKLVQDVIRQFLEAIYEPIFSDRSHGFR | | |
| | PNRSCHTALKQISRSFRGAKWFVEGDIKGCFDNI | | |
| | DHAVLLNLLSEKIKDSKFVTLIGKFLKAGYLEA | | |
| | WQYHATYSGTPQGGILSPILANIYLHELDKKVE | | |
| | QLKQDFSRPAEKVRTTIYSTKAREIERVRKLYA | | |
| | DCVSDEERKEVLDKIQKLKTEIRTLPYKDATDK | | |
| | KLAYVRYADDFIISVCGTREECEEIKQQLKSFLS | | |
| | EKLKLELSDDKTKITHSSENARFLGYDVNVRRN | | |
| | NECKRKGNGTIQRTLNNSVELLVPFEKIERFMFE | | |
| | RKIVKQDKDGTLIPWQRLSMYGLTDLEVLDTY | | |
| | NSQTRGICNYYSLASNFAKLKYFVYLMEYSCLK | | |
| | TLAQKHKTRISAIKRKYKAGHSWGIPYETKNGA | | |
| | KKMMSIKFSDLNKSAIFNGEVDKITHHAHFTNA | | |
| | NSLENRLKMKKCELCGADSNTTFEIHHINKLKN | | |
| | LKGKEQWERAMIARKRKTLVVCKSCHNGIHHS | | |
| | S | | |
| 8225 | KPTIEILTRLQENSKNNHEEVFTKLFRYLLRPDIY | OLA23482.1 | Faecalibacterium |
| | YVAYQNLYANNGAATKGVDEDTADGFSEDKV | | sp. |
| | NRIIEALRNGTYEPKPVRRTYIKKKNGKMRPLG | | CAG:74_58_120 |
| | LPTFTDKLVQDVIRMVLQAIYEPVFSNYSHGFRP | | |
| | GRSCHTALAQLKHEFIGAKWFVEGDIKGCFDNI | | |
| | DHSVLIGIVGKKVKDARFINLLRLFLKAGYMEE | | |
| | WKYYGTYSGCPQGGIISPILANIYLNELDTFVEK | | |
| | LKKSFDTNTPYTLTPQYRALQNKRANTKQKINR | | |
| | REVGEERDQLIAQYIGLGKELRKTPAKLCNDKK | | |
| | LKYVRYADDFLIAVNGSKEDCEWIKAQLTEFIR | | |
| | GTLKMELSQEKTLITHSNDCARFLGYDVRVRRD | | |
| | QQVKPWKNCKQRTMNNTVELLIPFRDKIEKYLF | | |
| | AKGAVKQRPDNGKLEPVARIGLTRNTDLEIVTT | | |
| | YDAELRGLCNFYYLASNYRNLNYFSYLMEYSC | | |
| | LKTLAWKHKCKLSKIYDKYRIGAKRWGIPYET | | |
| | KSGRKVRKLTKFNEVDGKRCEDAIPTIVTIIAKS | | |
| | RTTIDSRLKACRCELCGYEGKDRKYEVHHVNK | | |
| | VKNLKGKEPWEIVMIAKRRKTLVVCHECHQKI | | |
| | HHGY | | |
| 8226 | KPTMEILTKLQENSKKHHDEVFTRLFRYMLRPD | WP_ | Amphibacillus |
| | IYFVAYQHLYANRGAGTKGINEETADGFSEKYV | 017472863.1 | jilinensis |
| | EQIIEALRTETYRPKPVRRTYIKKSNGKMRPLGL | | |
| | PTFTDKLVQEVIRMILESVYEPIFSNNSHGFRSGR | | |
| | SCHTALTQIKNQFIGARWFVEGDIKGCENNIDHT | | |
| | ILTKIIGKKIKDARFIKLVHLFLKSGYMENWKYY | | |
| | GTYSGCPQGGIISPILANIYLNELDNFMEKIKQDF | | |
| | DNRTPYQLTAEYKKVMNKRSSLSQKIKRCEAG | | |
| | ARRDGFIEEYNNLSQQIYKIPAKLCNDKKLMYV | | |
| | RYADDFLIAVNGNKQDCEWIKAKLTEFIHNDLN | | |
| | MELSQEKTLITHSSICARFLGYDVRIRRSQQIKA | | |
| | WKKTKQRTMNNSVELLIPLEDKIQSFLFSRGIVR | | |
| | QRKDNGKMEPFRRNSLLRQTDLEIVSTYDAELR | | |
| | GICNYYSLAVNYSKLNYFSYLMEYSCLKTLATK | | |
| | HRTKISKIISKCRMANKRWGIPYQTKSGMKRKR | | |
| | LTKIYEIDRKKCEDIFPRAITIYAKGKTTFDDRLK | | |
| | AKVCEVCGRTDSERYEIHHVNKVKNLKGKEPW | | |
| | EQIMIAKRRKTMVVCHECHQKIHHGF | | |
| 8227 | KPTVEILTKLQENSKKHHDEVFTRLYRYLLRPDI | WP_ | Clostridioides |
| | YYEAYQHLYSNKGAGTKGITEDTADGFSEKYV | 022618695.1 | difficile |
| | ERIIELLKAETYLPKPVRRTYIKKSNGKMRPLGL | | |
| | PIFADKLVQEAIRMILEAVYEPVFIDYSHGFRPG | | |
| | RSCHTALAQIKKEFTGARWFIEGDIKGCFDNISH | | |
| | AVLVEVIGRKIKDARFLKLIRSFLKAGYMENWK | | |
| | YHETCSGCPQGGIISPILANIYLNELDQYIMKLK | | |
| | KDFDVTAKAPYTPEYSRIIWKRQRLHNRIKDSE | | |
| | GMEREQLIDEYKSATAQMFKIPAKLCEDKKIKY | | |
| | VRYADDFLIAVNGSRQECEVIKGQLTEFVHNTL | | |
| | KMELSQEKTLITHSNTPARFLGYDVRVRRDQQI | | |
| | KPKGRFKTRSMNNKVELNIPFKDRIEKFLFANGI | | |
| | VEQRKDNGKLEPCKRPQLLNMTDLEIVTVYNA | | |
| | ELRGICNYYGIASNFNKLIYFNYLMEYSCLKTLA | | |
| | NKHCSKISKVREMYKDGTGEWGIPYQTKKGMK | | |
| | RMYFAKYSDCKGKRFTDIIPQQAKNHSHNTTTF | | |
| | ESRLKAKACEICGCTDSDKYEIHHVNKLKNLKG | | |
| | KTKWEQVMIAKRRKTIVVCHKCHMVIHHGGK | | |
| | KE | | |
| 8228 | KSTMEILTKLQENSQKNQDEVFTRLYRYLLRPD | WP_ | Faecalibacterium |
| | IYFIAYQHLYSNKGAGTKGVNDDTADGFSEQY | 097783669.1 | prausnitzii |
| | VTAIIEALRTGSYEPKPVRRTYIQKKNGKLRPLG | | |
| | LPVFADKLVQEAIRMILEAIYEPIFSIYSHGFRPG | | |
| | RSCHTALAMIKHEFTGAKWFIEGDIKGCFDNID | | |
| | HSTLIGVLNRKIKDARFLNLIRMFLKSGYMEDW | | |
| | DFHETYSGCPQGGIISPILANVYLNELDRYITQL | | |
| | KKEFDHGYNPRNFTEEYNAIRHKRDALHEKIKK | | |
| | AEGTMREQLIAQHKQLTKQLFRTPAKACTDKR | | |
| | LKYVRYADDFLIAVNGTREECEAIKAKLTDFVR | | |
| | DTLKMELSQEKTLITHSNTPARFLGFDVRVRRD | | |
| | ASVKRSGKRKMRTMNNKVELNIPLKDKVETYL | | |
| | LSHSIAKRDRKRLIPIHRPILLNRTDLEIVMIYNA | | |
| | ELRGLCNYYAIASNFNKLVYFGYLMEYSCLKTL | | |
| | ANKHRSRISKVRYEYRDGTGAWGVPYETKKGK | | |
| | RRMMFAKYSDCKGKDLTEKVPDLAYRYSHNT | | |
| | TSFEERLKAKVCEVCGCTDSDSYEIHHVNKVKN | | |
| | LKGKADWEKVMLAKRRKTIVVCHKCHMRIHH | | |
| | GTKTE | | |
| 8229 | KPTTEILVNISKNSSKNKDEVFTRLYRYMLRPDL | 1947404.3. | |
| | YFIAYKNLYANKGASTQGIDNDTADGFSKEKID | peg.615 | |
| | RIIQSLSDESYQPKPVRRKYIQKKGNSKKKRPLG | | |
| | IPTFTDKLVQEVLRMILEAVYEPIFSNNSHGFRPE | | |
| | KSCHTALNSIKKEFTGTTWFVEGDIKGCFDNIN | | |
| | HHVLVDIIGRKIKDARLIKLVWKFLRAGYIEDW | | |
| | KYHTTYSGSPQGGIISPLLANIYLNELDKFAEKT | | |
| | AKAFYKKRDREHTKEYDAVMNALVLVKYHLK | | |
| | KATGQQKSDLLKQKKRLQRQLRKIPCSSQTDK | | |
| | VMKYVRYADDFIIGVKGDKIDCEKIKKQFADFI | | |
| | SQELKMELSEEKTLITHSSQFARFLGYDIRVRRD | | |
| | NTVKPHGTHLQRTMNMKVELCIPFQDKIMPFLF | | |
| | NKSIIRQLKDGTLEPIARKYLYSCTDLEILTAFNA | | |
| | ELRGICNYYALASNYNRLRYFAYFMEYSCLKTI | | |
| | AGKHKTTARKIISKYSYDGSWRIPYKTKEGIKYS | | |
| | KFADFMKCKKVTDFDEVIKDYAVMHASTRTTF | | |
| | EDRLSAEVCELCGKINAPLEIHHVNKVKNLKGK | | |
| | DFWEIMMIAKKRKTIAVCKECHHKIHHP | | |
| 8230 | QPTIEILDRIRKNSRDNKEEIFTRLYRYLLRPDLY | WP_ | Enterocloster |
| | YLAYKNLYANKGAGTKGVNDDTADGFSKEKV | 002592887.1 | clostridioformis |
| | DRIIQSLADGTYTPNPVRRKYIQKKQNSTKKRPL | | |
| | GIPTFTDKLVQEVLRMILESVYEPIFSNNSHGFRP | | |
| | NRSCHTALKSLKREFSGVSWFIEGDIKGCFDNID | | |
| | HQVLANVINAKIKDARLIQLIWKFLKAGYMED | | |
| | WQYHATYSGCPQGGIVSPILANIYLNELDKFVE | | |
| | KTAKEFYKSRDRHHTPEYDKVTWQIKKAQKQL | | |
| | KTATGQEKTALLQKIAQLKAVMHKTPCMSKTD | | |
| | KVIKYIRYADDFILGVKGDKADCGRIKRQLSDFI | | |
| | SQTLKMELSEQKTLITHSNQYARFLGYDIRVRR | | |
| | DQKLKPHGNHVSRTLNGSVELCIPFADKIMPFLF | | |
| | GKSVIRQLRDGTIEPTARKYIFRCTDLEIVSTYNS | | |
| | ELRGICNYYSIASNFNKLQYFEYLMEYSCLKTL | | |
| | AGKHESTSRKMMRKYRDGNGSWGVPYQTKAG | | |
| | IKRRSFARFMDCKNTDLWTDKIIDFAIAHIGSRT | | |
| | SFDDRLSARVCELCGKTNVPLEIHHVNKVKNLK | | |
| | GKQLWELAMIAKKRKTLAVCKDCHHKIHHP | | |
| 8231 | QPTTAILDRIMRNSRKNNEEIFTRLYRYMLRPDL | WP_ | Anaerotruncus |
| | YYLAYNKLYRNKGAATKGVDDDTADGFSEEKI | 016316325.1 | sp. G3(2012) |
| | NRIIQSLADETYMPKPVRREYIPKKRSSTKKRPL | | |
| | GLPSFTDKLVQEVLRMILEAVYEPTFSDFSYGFR | | |
| | PHRDCHTALKALKKEFTGVSWFIEGDIKGCFDN | | |
| | IDHQVLVGVISSKIKDARLIKLIWKFLKAGYMEE | | |
| | WKYHTTYSGCPQGGIISPLLSNIYLNELDKFAEK | | |
| | VARAFYKPRDRVRTPEYAKIQCKKDYAQKLLK | | |
| | TATGQKKVELLKRVKSLKSELRKVPCSSKTDKV | | |
| | MKYIRYADDFIIGVKGDKSDCEHIKRQFSDFISE | | |
| | HLKMELSEEKTLITHSNQYARFLGYDVRVRRD | | |
| | GKVKPTDRCLKRTLNYTVELNVPFADKIMPFLF | | |
| | DKAIIKQTHDGKIEYIARKYLYRCTNLEIIDTYNS | | |
| | ELRGICNYYSIASNFTSLNYFAYLMEYSCLKTLA | | |
| | GKHKSTSRKIREQFRTGSGDWGIPYNTAKGQQK | | |
| | YRTFAKYMDCKDSDRENDVIVECAIRHAGTRTT | | |
| | LEKRLSAGICELCGKTNTPLAMHHVNKVKNLK | | |
| | GKQQWEIVMIAKRRKTLAVCKDCHYKIHHP | | |
| 8232 | KPTMEILERIKKNSEENKDEVFTRIYRYLLRPDI | MBS4931873.1 | Clostridiales |
| | YFVAYQNLYSNNGASTKGVDDDTADGFSEAKI | | bacterium |
| | ERIIKCLEDESYQPKPFRRVYIKKPNGKMRPLGI | | |
| | PSFTDKLVQEAVRIILEAIYEPIFMDTSHGFRPNR | | |
| | SCHTALQSVKYEFRGARWFIEGDIKGCFDNINH | | |
| | NVLVSCINKKIKDARFTKLIYKFLKAGFVDDFV | | |
| | YNNTYSGCAQGGIISPILANIYLHELDKFVENLS | | |
| | KEFNEPATEKFTADYRKAQNAMAVTRKKIKKA | | |
| | ENADDEVEKAELLKVYKSQRATLLKTPCKSQT | | |
| | DKKLKYVRYADDFIIGVNGSKVDCVRIKQQLSD | | |
| | FISNTLKMELSEEKTLITHSNTYAKFLGYNIRVR | | |
| | RSNTVKPNGRGATQRTMSNGVELAIPLKEKING | | |
| | FMFKNGIVKQCDNGELEPVCRNDMLRLTDLEIV | | |
| | SGYNAELRGICNYYYMASNFYMLNYFSYLMEY | | |
| | SCLKTLAGKHRCSIGKIKEKFSDHKGKWCIAYE | | |
| | TKKGTSYLYLSKYSDCKKGKNATDTRTSMVQI | | |
| | HKNTRSTFESRLKAKCCELCGSTTSNQYEIHHV | | |
| | NKIRNLKGKEPWEIMMLSKRRKTMVVCWECH | | |
| | KKIHNQNFEVKQ | | |
| 8233 | AEMQPTTEILTRISKNSLNNKDEVFTRLFRYLLR | ERJ86739.1 | Ruminococcus |
| | EDIWFEAYRNLYANNGASTKGVNDDTADGFSE | | callidus ATCC |
| | RKIQKITEQLKNGKFNPTPVRRTYIQKKNSDKM | | 27760 |
| | RPLGIPTFTDKLVQEAVRMILEAVYEPIFHECSH | | |
| | GFRPNRSCHTALKSLRMKFTGAKWFIEGDIKGC | | |
| | FDNINHDVLIGILNKKIKDARLIQLIQQFLKAGY | | |
| | LEDWIYHRTYSGTPQGGIISPILANIYLHELDKFV | | |
| | ENLKEEFDKPSKEKYTLEYRKAKYQTEKARKAI | | |
| | RECDPQDYERKKQLIKNLKAVRSVQLKTPCKSQ | | |
| | TDKKIQYIRYADDFILSVNGSREECIEIKKKLSQY | | |
| | ISEVLKMQLSDEKTLITHSSNHARFLGYDISVRR | | |
| | NAKIKSKNGGVSLRTLNNKVELLIPLKEKINRF | | |
| | MFDKGVIFQKKDGSLFPTHRSYMIHMSDLEIIST | | |
| | YNSELRGICNYYNLASNYCQLRYFAYLMEYSC | | |
| | LKTLAAKHNTKISKIIAKFKDGKGGWGIPYETK | | |
| | SGKKRCYFAKYSDCKDSKDGTDNISNAAVIYG | | |
| | YSRNTLEERLKAKVCELCGDTNAEYYEIHHVH | | |
| | KVKDLKGKNDWERAMIAKRRKTLVLCRNCHH | | |
| | KVHNQ | | |
| 8234 | AEMLPTTEILTRISKNSLKNPNETFTRVFRYMLR | ETA80462.1 | Youngiibacter |
| | PDIWFLAYKNLYANNGASTKGINNDTADGFSE | | fragilis 232.1 |
| | KTISNIIKSLENGEFCPTPVRRTYIAKKSSDKKRP | | |
| | LGIPTFTDKLVQEVLRMVLEAIYEPVFMDCSHG | | |
| | FRPNRSCNTALKSLRLKFTGAKWFVEGDIRGCF | | |
| | DNIDHSVLIRLLNQKIKDERLIQLIYKFLKAGYM | | |
| | EDWTYHRTYSGTPQGGIFSPVLANIYLHELDKFI | | |
| | VNLKNEFDKPSAELYTVEYRKAQWQTVKARK | | |
| | AIKNCDPNNKIQKKQFIKEMKSVRSVQLKTPCK | | |
| | SQTDKKIQYIRYADDFIIAVNGSREDCVEIKNKL | | |
| | SLFISSALKMQLSEEKTLITHSSNYARFLGYDVCI | | |
| | RRNAKVKPKKGGITVRTLNNKVELLIPIKDKLN | | |
| | KFLFNKGIVYQKKDGTLFSTHRTSLIRLSDLEIVS | | |
| | TYNSELRGICNYYSLASNYCQLRYFAYLMEYSC | | |
| | LKTLAAKHNSYISKIINKFQNGKGEWGIPYETK | | |
| | QGPKRCYFAKYSDCKSGKDYTDKITKAAIIYGF | | |
| | SRNTLEERIKAKVCELCGKTNADHYEIHHIHKV | | |
| | KDLKGKADWERAMISKRRKTMVLCRNCHHKI | | |
| | HNQ | | |
| 8235 | KPTTEILARISQNSLANKEEVFTKLYRYLLRPDI | WP_ | Streptococcus |
| | YFVAYKNLYANNGAATKGVNEDTADGFSEAKI | 069987880.1 | agalactiae |
| | DSIIKALADETYQPMPVRRTYIQKKNNRKKLRP | | |
| | LGIPTFTDKLVQEVLRMILEAVYEPIFLDVSHGF | | |
| | RPKRSCHTALKQLRREFNGTRWFVEGDIKGCFD | | |
| | NINHAVLVGLLSNKIRDARITKLIYKFLKAGYLE | | |
| | NWQYHKTYSGTPQGGIISPLLANIYLHELDKFV | | |
| | MKLKSEFDTPGVGQITPEYRELHNEIKRLSHRLT | | |
| | KVTGEEREMVLAEYKPKRQKLMTIPCTAQTDK | | |
| | KLKYVRYADDFLIAVKGNREDCQWIKSKLAEFI | | |
| | GDTLKMELSEDKTLITHSSKCARFLGYDVRVRR | | |
| | SGKIKRGGPGHVKMRTLNGGVELLVPLNDKIR | | |
| | QFVFTKGVAIQKEDGSMFPIHRKYLVGLTDLEI | | |
| | VSVYNAELRGICSYYGMASNFCKLHYFSYLME | | |
| | YSCLKTLASKHKTSLSKIIDKCNDGTGKWGVPY | | |
| | ETKLGSKRRYFANYADCKGKGSATDYISNAAV | | |
| | VYGYAVNTLENRLKAKVCELCGTTESDHYEVH | | |
| | HINKLKNLKGKERWEIAMIAKHRKTLVVCRDC | | |
| | HRSIIHKK | | |
| 8236 | QPTTEILARISKNSLANKEEIFTKLYRYLLRPDLY | WP_ | Eubacteriales |
| | FLAYNHLYANNGAATKGANNDTADGFSEVKIA | 021642534.1 | |
| | NIIKSLSDDTYQPTPVRRIYISKKSDPKKKRPLGI | | |
| | PTFTDKLIQEALRMVLEAVYEPVFLNASHGFRP | | |
| | KRSCHTALTSLKKEFNGTRWFVEGDIKGCFDTI | | |
| | DHATLVGFVNNKIKDARIIKLIYKFLKAGYLED | | |
| | WQYHKTYSGTPQGGIISPLLANIYLHELDKYVM | | |
| | KLKAEFDAPNTEKITPEYRELHNEIKMLSYYIKK | | |
| | ADGTEKERLLAEYKPKRKRLMSIPCTSQTDKKI | | |
| | KYVRYADDFIIGVKGSQEDCQWIKSKLAEFISET | | |
| | LKMELSEEKTLITHSSECARFLGYDVRVRRSGEI | | |
| | KRGGPGNAKKRTLNNHTELLVPLNDKIHKFIFS | | |
| | KGIAIQKIDGTLFPVHRNSLLRLTDLEIVTAYND | | |
| | ELRGLCNYYGMASNFHKMKYLAYLMKYSCLK | | |
| | TLASKHKSSISKVIAMFKDGKGDWGIPYETKAG | | |
| | AKRRYFVNYIDCKEAKNPTDIISNAAVIYGQSVT | | |
| | TLEKRLKARVCELCGTAESDHYEIHHVNKLKNL | | |
| | KGRKQWEIAMLAKRRKTLVVCEKCHHEIHNQ | | |
| 8237 | QPTTEILERISKNSLTHKEEVFTRLYRYLLRPDIY | WP_ | Faecalibacterium |
| | YQAYQRLYTNKGASTKGANQDTADGFSEAKIE | 087385514.1 | sp. An122 |
| | KIIQSLADETYQPTPVRRTYIAKKNNPKKKRPLG | | |
| | IPTFTDKLVQEALRMILEAIYEPLFLDCSHGFRPK | | |
| | RSCHTALEKLKYQFGGVRWFVEGDIKGCFDNIN | | |
| | HEALVGFIGNKIKDARIVKLVYKFLKAGYLEDW | | |
| | VYHKTYSGTPQGGILSPLLANIYLNELDQFVMK | | |
| | LKDEFETPEKGQITPEYRALHNKIKNLCYHIDRK | | |
| | QGVEKERMIAECKVLRKQLLKTPCTAQTDKKL | | |
| | KYIRYADDFIIGVKGSKEDCQWIKSKLAEFIGQT | | |
| | LKMELSEEKTLITHSSQCARFLGFDVRVRRCEK | | |
| | VKRNKKGAKMRTLNNHVELLVPFDDKIHDFIFS | | |
| | KKIAIQKKDGKLFPVHRNSLLRATDLEIVTVYN | | |
| | DELRGICNYYGIASNFCKLKYLSYLMEYSCLKT | | |
| | LAAKHKSKISKVVAMYKDGTGEWGIPYETKKK | | |
| | SKRRYFANYMDCKNAKNPTDQISNAAIIYGQSV | | |
| | TTLEKRLKARVCELCGTTESEHYEIHHINKLKNL | | |
| | KGKEPWEIAMLAKRRKTLVVCERCHHLIHNQ | | |
| | KPTMAILERISKNSMEQKDEVFTRLYRYLLRPDI | | |
| 8238 | YYIAYQNLYSNKGAGTKGIDDDTADGFSEKKIS | WP_ | Streptococcus |
| | TIINSLASESYTPKPVRRTYISKKSSSKLRPLGLPT | 014622875.1 | equi |
| | FTDKLIQEVLRLILEAIYEPIFLDTSHGFRPKRSC | | |
| | HTALKMIKREFGGARWFVEGDIKGCFDNIDHQ | | |
| | VLISIIQKKVKDARFIKLIYKFLKAGYMENWNY | | |
| | HKTYSGTPQGGILSPLLANIYLHELDLFVLKLKE | | |
| | QFDNPQKDNITSEYRQAHNELKRLSNRLKKVEG | | |
| | NEKQELLEEYLIKRQRLMTIPCTAQTDKKLKYV | | |
| | RYADDFIISVKGNKKDCHWLKQQLADFINGHL | | |
| | KMTLSPEKTLITHSSNCARFLGYDIRVRRSQAIK | | |
| | RGGSGQVKKRTLNGSVELLIPFKDKIHLFLFNK | | |
| | GIVIQKNDGSYFPVHRKNILTATDLEIVTIYNSEL | | |
| | RGICRYYGLTSNFNQLNYFAYLMEYSCLKTLAS | | |
| | KHKTSLVKIRAKYKDGFGSWAIPYETKTTKKR | | |
| | MYFTDYTKCKSPSTFTDLKSSVAVTYGYSRTTF | | |
| | ESRLKAKKCELCGTTDKQTTYEIHHVNKGKNL | | |
| | KGKEKWEQMMIAKQRKTLVVCHHCHRHVIHN | | |
| | H | | |
| 8239 | KPTMAILERISKNSQENIDEVFTRLYRYLLRPDIY | WP_ | Lactococcus |
| | YVAYQNLYSNKGASTKGILDDTADGFSEEKIKK | 011835237.1 | cremoris |
| | IIQSLKDGTYYPQPVRRMYIAKKNSKKMRPLGIP | | |
| | TFTDKLIQEAVRIILESIYEPVFEDVSHGFRPQRS | | |
| | CHTALKTIKREFGGARWFVEGDIKGCFDNIDHV | | |
| | TLIGLINLKIKDMKMSQLIYKFLKAGYLENWQY | | |
| | HKTYSGTPQGGILSPLLANIYLHELDKFVLQLK | | |
| | MKFDRESPERITPEYRELHNEIKRISHRLKKLEG | | |
| | EEKAKVLLEYQEKRKRLPTLPCTSQTNKVLKYV | | |
| | RYADDFIISVKGSKEDCQWIKEQLKLFIHNKLK | | |
| | MELSEEKTLITHSSQPARFLGYDIRVRRSGTIKRS | | |
| | GKVKKRTLNGSVELLIPLQDKIRQFIFDKKIAIQ | | |
| | KKDSSWFPVHRKYLIRSTDLEIITIYNSELRGICN | | |
| | YYGLASNFNQLNYFAYLMEYSCLKTIASKHKG | | |
| | TLSKTISMFKDGSGSWGIPYEIKQGKQRRYFAN | | |
| | FSECKSPYQFTDEISQAPVLYGYARNTLENRLK | | |
| | AKCCELCGTSDENTSYEIHHVNKVKNLKGKEK | | |
| | WEMAMIAKQRKTLVVCFHCHRHVIHKHK | | |
| 8240 | KPTMAILERISKNSQENIDEVFTRLYRYLLRPDIY | YP_796487 | Lactococcus |
| | YVAYQNLYSNKGASTKGILDDTADGFSEEKIKK | | lactis subsp. |
| | IIQSLKDGTYYPQPVRRMYIAKKNSKKMRPLGIP | | cremoris SK11 |
| | TFTDKLIQEAVRIILESIYEPVFEDVSHGFRPQRS | | |
| | CHTALKTIKREFGGARWFVEGDIKGCFDNIDHV | | |
| | TLIGLINLKIKDMKMSQLIYKFLKAGYLENWQY | | |
| | HKTYSGTPQGGILSPLLANIYLHELDKFVLQLK | | |
| | MKFDRESPERITPEYRELHNEIKRISHRLKKLEG | | |
| | EEKAKVLLEYQEKRKRLPTLPCTSQTNKVLKYV | | |
| | RYADDFIISVKGSKEDCQWIKEQLKLFIHNKLK | | |
| | MEFSEEKTLITHSSQPARFLGYDIRVRRSGTIKRS | | |
| | GKVKKRTLNGSVELFIPLQDKIRQFIFDKKIAIQK | | |
| | KDSSWFPVHRKYLIRSTDLEIITIYNSELRGICNY | | |
| | YGLASNFNQLNYFAYLMEYNCLKTIASKHKGT | | |
| | LSKTISMFKDGSGSWGIPYEIKQGKQRRYFANFS | | |
| | ECKSPYQFTDKISQAPVLYGYARNTLENRLKAK | | |
| | CCELCGTSDENTSYEIHHVNKVKNLKGKEKWE | | |
| | MAMIAKQRKTLVVCFHCHRHVIHKHK | | |
| 8241 | NPTSEILERVNKSSSEHHDGVFTRLFRYLLREDI | 1638786.3. | |
| | YFAAYQKLYANSGAMTPGSDNDTADGFSAEYV | peg.2502 | |
| | HELIEELRSGKYKPKPVRREYIKKQNGKMRPLG | | |
| | IPSFRDKLLQEAVRMFLEAIYEPLFYDQSHGFRP | | |
| | ERSCHTALDQIKTNFRSVKWFIEGDIKGCFDNID | | |
| | HAVLIKTLEVKIKDSRFINIIRAFLKAGYVEDFQ | | |
| | YHTTISGTPQGGIISPILANIYLHELDRKVMKLKE | | |
| | KFDKQSTRHQTPEYLHLAKRRQTLQKKIDRVK | | |
| | GEERELAIKEYKAVCNQKLKTPARMSDDKKLV | | |
| | YCRYADDFLIGISGSREDCEEIKEILREFLSTQYH | | |
| | LELSAEKTKITHSAERVRFLGYDVAVRRSQKIK | | |
| | KKANGVKQRTLNNSVELTVPLEDKIMQFLFKN | | |
| | DIIGQKPNGEIWAVCVPRLRHLSEVDIVNRYNA | | |
| | QIRGICNYYCLAANYDKLNYFRYLMEYSCLKTL | | |
| | ASKSNSTTRKIIQKYRHDGKWAIPHEVKGGIKY | | |
| | AKLVSLADCKAGKLMSDKDPWQYKSFDPKKLS | | |
| | QYVRLSAGVCELCGDNSDSCCIYHAGKMKNLK | | |
| | STTEWGKKMLHMRRKTLIVCPKCFKKIHREQN | | |
| | K | | |
| 8242 | KPTFEILERIEKCSTKYVDGVFTRIYRYLLREDIY | WP_ | Aerococcus sp. |
| | HAAYQNLYANKGATTKGIDEDTADGFSNEYVQ | 070626229.1 | HMSC072A12 |
| | ELINSLKDGSYKAKPVRREYIPKQNGKLRPLGIP | | |
| | TFRDKLLQEVVRMILEAIYEPIFHKNSHGFRPGK | | |
| | SCHTALKQIKTEFTGVVWFIEGDIKGCFDNINHN | | |
| | KLIEILGRKIKDSKFLNIIRQFLKAGYIENWQYN | | |
| | ATYSGAPQGSICAPILANIYLNELDKKFDEISTHF | | |
| | DKPSSAYKSPKYHEVDKEMKRLSYWIDNTTDE | | |
| | EERQELIKQYKEQKKSLRTLPCKNKDNKRFTFV | | |
| | RYADDWLVGVCGTKEDCKDLKEEIAKFLDEEL | | |
| | KLTLSEEKTLITHSSEKVRFLGYDISVRRNKQVK | | |
| | GHKMKNGKWRQSRTLHMKVALTIPHSDKIEKF | | |
| | MFDKGVIRQKENGEIQPIHRAGLLNLSDSEIVEH | | |
| | YNAEARGLCNYYKLAVDYHTLGYFCYLMEYS | | |
| | CLKTIANKHKTSIRKIINKYKDGKTWSVPYETK | | |
| | AGTKRVKPVKIADCKGGKVEDIIFVRKKENWK | | |
| | TTIRQRLNAKTCELCGCKNAELYEVHVVKNLK | | |
| | DLGDSNWEQAMKEKRRKTLVVCNKCHKEIHE | | |
| | H | | |
| 8243 | KPTSEILERIAKSSTEHKDGVFTRLYRYLLREDIY | WP_ | Streptococcus |
| | YAAYQKLYANRGATTKGIDDDTADGFSAHYIK | 044681649.1 | suis |
| | ELIHDLENGTYRANPVRREYIPKKNGKMRPLGI | | |
| | PSFRDKLLQEVVRMILEAIYEPVFDDHSHGFRPN | | |
| | RSCHTALRQISSDFTGVVWFIEGDITGCFDNIDH | | |
| | EILIDILARKIKDSKFLNVIRQFLKAGYVENWKY | | |
| | NKTYSGTPQGGIVSPILANIYLNELDKKFNEIKR | | |
| | RFDEPRTSRHEKTPKYREIDNEMKKISYWIDHT | | |
| | DDDEKRKELVKQFKQLKKEIHTIPCHPQTHKKF | | |
| | TFVRYADDWLVGVCGTKEECIALKAEIADFLSK | | |
| | ELKLTLSEEKTLITHSSEKVRFIGYDICVRRSQEI | | |
| | KGYKMKNGKWRKSRSLHLKVALTIPHTEKIEK | | |
| | FLFAKKAIIQTNGGALKFKPVHRTALLNLSDSEI | | |
| | VEHYNAEMRGILNYYNLAVDYHTLDYFCYLM | | |
| | EYSCLKTIANKHKTSIRKIVRLYKDGNTWSVPY | | |
| | ETKEGTKRVRPIKIADCKRGEASDIVFQRTKFN | | |
| | WKSTIRQRLNAGVCELCGKKHADLYEVHVVRN | | |
| | LNELGNSDWELAMKSKRRKTLVVCSDCHRRIH | | |
| | K | | |
| 8244 | KPTSMILERIAKSSTEHKDGVFTRLYRYLLREDI | 1950830.3.pe | |
| | YFAAYQKLYANKGATTKGIDNDTADGFSSKYV | g474 | |
| | NDLIQELKNGTYQANPVRRVYIEKKNGKLRPLG | | |
| | IPSFKDKLLQEVVRMILEAIYEPVFDKNSHGFRP | | |
| | NKSCHTAMKQISSEFTGVIWFIEGDIKGCFDNID | | |
| | HQILINIIAKKIKDSKFLNIIRQFLKAGYIENWKY | | |
| | NATHSGTPQGGICSPILANIYLNELDNKFREIQG | | |
| | KFNKARTIEEIKTLEYRTIDNEMKRVSYWINHTE | | |
| | NEQERNNLIKKYKALQQEIHKVPCHTKNNKKFT | | |
| | FVRYADDWLAGVCGTKEECVMLKAEIAKFLTE | | |
| | ELKLTLSEEKTLITHSSQKVRFLGYNINVRRSKE | | |
| | VKGFKMKNGKYRKSRTLHYKVALTIPHKEKIE | | |
| | KFLFSKGVIMQKANGEIKPIHRTVLLNLSDKEIL | | |
| | EQYNAEMRGILNYYRLAVDYHTLNYFCYLMEY | | |
| | SCLKTIANKHKSSIRKIIREYKDKNTWSIPYETKT | | |
| | GIKRIRPVKIADCKKGVVNDVIYKRTNFSFKSTI | | |
| | RQRLNARTCELCGQTGNELYEVHTIKNLNELGN | | |
| | LNWEKAMKKMKRKTIIVCKECHNIIHS | | |
| 8245 | KTTCEILERIQKNSTEHKDGVYTRLYRYLLREDI | WP_ | [Clostridium] |
| | YYVAYQRLYSNKGATNKGATTKGVDNDTADG | 021420371.1 | innocuum |
| | FGQVYVQELITQLRNGTYKPKPSRRVYIEKSNG | | |
| | KMRPLSIPSFRDKLLQEVVRMFLEAIYEPIFSDYS | | |
| | HGFRPNRSCHSALKQAKIYFTGAKWFIEGDIKG | | |
| | CFDNINHKVLINILERKIKDSKFINIIRLFLTAGYV | | |
| | DDFKYNATYSGCAQGGIISPILANIYLNELDKKI | | |
| | LEIKNKFDKPHQAKYTKEYSHIKSKRDYQKSKL | | |
| | KNCDEEQRKEILRTIDDLNKKLRKTPRTPNDDK | | |
| | NIYFIRYADDFLIAVKGNKNDCEIIKKEIHDFLRD | | |
| | ELKLTLSEEKTLITHSSNKALFLGYNISIRRSQTV | | |
| | KSVSQNGRKYKQRTLNNSVALTVPFERIEKFMF | | |
| | KRRMIKQIKPKTFRPLHRKGWLYLPDYVIVERY | | |
| | DAELRGILNYYNLAVDYNYLGYFRYLMEYSCL | | |
| | ATIAGKHNSSTSKIVSQYRHGKYWGVPYLINK | | |
| | GEEKIKRLARLKDCKSNACNDTIVKHRYVKAT | | |
| | NASIRDRLQTGVCELCGKRIDVPLEVHIVSKLKD | | |
| | LKDDKPWKVVMKSKRRKTLVVCPECHKHIHVE | | |
| 8246 | TKPTSDILERIYKNSSEHKDGVYTRLYRYLLRD | WP_ | Ruminiclostridium |
| | DIYYLAYQKLYSNKGASTKGIDNDTADGFGKK | 024832200.1 | josui |
| | YVDSLIKELSDGTYTPKPVRREYIKKKNGKMRP | | |
| | LGIPSFRDKLLQEVIRNFLEAIYEPTFSDFSHGFR | | |
| | PKRSCHTALEQAKLYFRGAKWFIEGDIKGCFDD | | |
| | IDHDKLIEILQRKIKDSRFINVIRSFLKAGYMED | | |
| | WKYHQTYSGCPQGGILSPILANIYLNELDNEIAK | | |
| | IKQAFDKPATRKITPEHSSLSAKLFKRRKKLKSA | | |
| | TGEQRTALLSEIHDLEEQYRKTPSKMQDDKKVS | | |
| | YVRYADDFLIAENGSKEDCVRLKEQLAKFLFDE | | |
| | YKLTLSKDKTLITHSSERVRFLGYDISVRRNQEY | | |
| | MTDSRGRKARHLNNTVALSVPFEKIEKHMFEK | | |
| | GFVRQTEAKKFRPLHKKGWLYLPDAEIVERYN | | |
| | AEIRGIVNYYYLASNLYKLQYFAYLMEYSCLAT | | |
| | LAGKHNSTIKKIVAKHKQGKDWAIKYKTENGA | | |
| | TKEKRIVKLKDCKGKCEDKIVQHRYSVNTNATI | | |
| | RARLQAGICELCGSKDKASYEVHHVPSVKGLD | | |
| | GTSLWEQIMKSKRRKTLVVCEDCHKAIHDD | | |
| 8247 | KPTAEILERINKNSNEHKDGVYTRLYRYLLREDI | WP_ | Petroclostridium |
| | YYSAYQKLYSNKGASTEGIDNDTADGFGKKYV | 094550212.1 | xylanilyticum |
| | ESSIEELSNNTYKPKPVRREYIKKSNGKMRPLGI | | |
| | PSFRDKLLQEVMRRFLEAIYEPIFSDFSHGFRPN | | |
| | RSCHTALKQTLPYFKGARWFIEGDIKGCFDNID | | |
| | HDKLIEILQRKIKDSKFINIIRSFLKAGYIEDFRYN | | |
| | QTYSGTPQGGILSPILANIYLNELDNKIMEIKQNF | | |
| | DKPATRCVNPTYDEIRGKRYWLQQKLKNATDE | | |
| | EKPVLISRINEYSKKLLKLPYKSQTDKNIAFVRY | | |
| | ADDFLIAVRGNKEDCIKIKEQLREFLNDELKLTL | | |
| | SDEKTLITHSSEKVRFLGYDISVRRNQQISTNSL | | |
| | GHKKRQLNGTVELLVPLEKIEKFMFDKGIIRQS | | |
| | KAKKFHPIHRKGWLYLPDQEILERYNAEIRGILN | | |
| | YYHLANNYNKLNYFQYLMEYSCLATLAGKHN | | |
| | SSISKVIDKYKSGKGWAIKYKTEKGKTREKRIV | | |
| | KLQDCKGFCDDNIVRHIYSVNTNATIRARLQAG | | |
| | VCELCGSRGKSNYEVHHVSSVKGLEGNKLWEQ | | |
| | IMKIKNRKTLVVCEDCHKAIHS | | |
| 8248 | LERALQQMRERADWRYLQSETQYVPWRAPGV | 19533 | |
| | DNMTLAYAADHLDEIITQRLERLSCLPYGPLPA | | |
| | KRYYIEEGSKQRPIAIMTVPDGIVSRALLELVRE | | |
| | PLEEPLPPCNFAYLQGIGPQRRVDHITSMVEQYG | | |
| | WVVQLDIRSYFDSIPHDLLYERIDRLIVDPDLLA | | |
| | LLWEFVTQPIRENGCDHATTVGVPQGGVISPVL | | |
| | ANLYLSPLDEAMLAEGWGYARYADDFVIFTST | | |
| | KAEARRARDYATEIIAELGLQVHRTGRKQAIIA | | |
| | KDCDGFEFCGHFYKWYGDRVYVAPRRSKIEEV | | |
| | V | | |
| 8249 | ETSVRHLGELTYPLRASAAFQRQALTGEPDLLT | WP_ | Buchananella |
| | EIAAPDSLLNAWRYVFTRDAKDGYLLQQSQQIA | 073825178.1 | hordeovulneris |
| | ADPDRFVAALSGALLSGRYQPEPQVEVLIPKKG | | |
| | KTSAMRELSIPSIRDRVVERAVLNAIIDRADLLQ | | |
| | CSASFAFRRGLGVQAATHEITQLRDSGNRYVLL | | |
| | TDIANYFGRINIADSLRVLQRGLFCSRTLALLRFI | | |
| | AKPRRVVGRRRIRSRGLAQGSCLSPLLANLALT | | |
| | DIDFALADTGVGYVRFADDILLCAPSRTELAAS | | |
| | QRLLASLAAHQGLQLNEEKTMHTSFDAGFCYL | | |
| | GVDFTAHQPVTDLHYGVKHTKQPAKV | | |
| 8250 | WFADEPRHTRGGSRMADLYRQVRLMKTLSSA | RCG92311.1 | Pseudomonas |
| | WRVVRASCMQSSSSEIRNEAIEFEADSFRQLKSI | | aeruginosa |
| | QSKLQKKKFEFLPQHGIAKKRPGKSSRPLVIAPI | | |
| | PNRIVQRAILDVLQDNVAYVQEILKVETSFGGIK | | |
| | GKNVALAIAAINKAFSNGVTHYVRSDIPSFFTKV | | |
| | QRAKVVDALAKNIDDVDMVNLFSAAIETTLGN | | |
| | LTDLQRRGLESIFPLSHDGVAQGSPLSPLIANIYL | | |
| | AEFDREMNREGLACIRYIDDFVIMAASEKQVM | | |
| | KGFRAAKAVLRRQGLQVYSPDDDPLKASKGDV | | |
| | RDGFDFLGCYVKPGLVQPSKFARNRLLEKID | | |
| 8251 | ATYDNFLLAWQRTVNTTSRMIRDELGMKIFAH | WP_096673502 | ischerella sp. |
| | NLQTNLEYLVQQVKAKDFPYKPLADHKVYVPK | | NIES-4106 |
| | PSTTLRTMSLMAVSDVIIYQALVNIIADKAYSYL | | |
| | VTHENQCVLGNIYSGPGKRWMLRPWKKQYTR | | |
| | FVDCIENLYHAGNPWIASTDIVAFYDTIDHARLL | | |
| | SLIRKYCGDDQQFQELLQECLAKWAVHNSNIT | | |
| | MGRGIPQGSNASDFLANLFLYEIDKEMIVNGYH | | |
| | YIRYVDDVRILASDKSTVQRGLILFDLELKRAGL | | |
| | VAQVTKTSVHEIEDIETEISRLRFIITAPTRNGNC | | |
| | LLVTLPSLPKSEQA | | |
| 8252 | DAEYLKSVWKSDIRPLLRQAKFSNSRYAIDPLH | WP_079554060 | Arthrobacter sp. |
| | YAAYEWNLDAFVDGIVRDLKLHQFTPERGEVIR | | 49Tsu3.1M3 |
| | AAKGTGLTRPVCELSPRDALVYTAIVKRVEDQL | | |
| | LVSSRKWVGHTRSDKGSSVETGDGAVDSFDWF | | |
| | QFWLRRQGLIADILEIDGVKFIVESDISNFFPSIRL | | |
| | EHVREHLLAHTRLSKELVRLCMQMIDGVLPRS | | |
| | NYLDYSHLGLPQGNNDSSRAIAHSFLAPIDQEFD | | |
| | VEGLAGRYTRYMDDVLYGVRHVAEGEKIISRL | | |
| | QRSLESLALTPNSAKTKIVPVDEYLRDSMVESN | | |
| | AEIERIQSLLESSGALGGSTEPEAKL | | |
| 8253 | AVWENIVEAERISTNRKMRNPGVIRHIGNRWRN | WP_ | Prevotella sp. |
| | LIEIQQFVLNGTMRTDEYQHEQRVSGQDKLRDI | 091853483.1 | BP1-145 |
| | AKLHFHPSHIQHQLITMAGNRRIDRSLIRHTYAS | | |
| | RKGYGQILCATEMKKSLSKYRRTERWYGQGDV | | |
| | CKYYDNIPHSLIREDLERLFKDKKFVDTFMEPFE | | |
| | RFAPEGKGIPLGIRPSQSIGNLTLKDFDHFMTEE | | |
| | NKCADYKRYLDDFMFTGATKGEVKRKMKRAI | | |
| | KYLHDLGFNTHEPKIHRISEGMDMLGFVYYGV | | |
| | KNDMWWRKSDKKRWLRHR | | |
| 8254 | AYDYYLHRKEKRDQKSGKSSNVITLKQIADHE | WP_ | Gimesia maris |
| | YLLYCFQELRRYGGLGAGKDDISYFDISTSDCA | 002646604.1 | |
| | KVFRKLSESLLRGRYRPQFPRKVPIPKPGTDEKR | | |
| | TLKINSIFDRSVSMALDKTLAPQLEKLFLEGSYG | | |
| | NRTNRSPWKMLAQLKKTVEETGRWVLAIEDIR | | |
| | KAFDNVKVKDIVKTHQQAQLELKEKHGIKINDS | | |
| | VVNLISTIAKGVTQKRKKGIDQGSNYSPQSLNV | | |
| | LLHYIHDVPLNAEVAFPLWYRYVDNLTYLCKS | | |
| | VSEGQRVLIKVRQVLNSASLKLKGEDGIVDLRK | | |
| | TTSSLLGFKLRRSNNQLIYLIAPRSWENLK | | |
| 8255 | SYFYKERRGKIYESYYGSIVTYNTTTSKMADSK | WP_ | Thermoanaerobacterium |
| | ELFSSFFKARMSLKKEFPYDEIAIKLFEYNLEDNI | 014757544.1 | |
| | NRFSKEILKGYKFNTDFIGYKVPKNEKDDRQKV | | |
| | MDNIFNTIAGASFLDIIGIVIDREFSSNCCGNRLN | | |
| | KKLNTEYSYEYFWYGWYYKFMKKAFNKVLNK | | |
| | NNYYLKLDIKSFYTNINQNILYDKIIKLIPYKDSR | | |
| | LKEFINSLIKRHIPYVNNGKGLPQGSLTSGFLAN | | |
| | LYLDDFDKYFISKTNDGYMRYVDDIFIFGKTEE | | |
| | QIKELGKEAENKLKDLYLEINKEKTSMGDKSSL | | |
| | KNIYYDDKELDDFQKRL | | |
| 8256 | ARLEAEGQHRQAKRLNRMYCKSFDTKLVAAT | WP_ | Methylobacterium |
| | DANKRLPAGQRAKRAELHEIAAGMDLRQTQGT | 170855116.1 | sp. |
| | ATFRAEPKKKGYRPVVNFDLRGRTAQLVLKRA | | 275MFSha3.1] |
| | AKPFIKIRPDQYASDGGQPAACHRIIELAAQGYA | | |
| | WFEEIDVRSFYASIIPEGVTELLGDLPKEMTEAN | | |
| | TLAKRVRASFMKTARDIPSDDLCKLRNEVRAGI | | |
| | PQGSALSPLVAEAVMSNVLDQATQGADWPDV | | |
| | QLVVFADNIAVLGRTKADVEDAAENLAGAFSR | | |
| | SQLGPFNLHRKPARSINQGFDFLSTRFIARNRRIR | | |
| | AEVAPAARLKRIH | | |
| 8257 | PKGFSFKDTFTPIQRKESLIGLLGIKDIEKFESLLR | WP_ | [Photorhabdus] |
| | DGVENAYYIKPPIKKKNGGERIVYAPNRMLKSI | 011148932.1 | |
| | LRKINNRIFNQINFPDYLYGSIPDKENPRDYILCA | | |
| | HQHCKAKILIKLDIENFFPTMKTKFVFNIFKDLF | | |
| | KFSDEVSNILTKLTTYDGFVPQGAPTSTYLANL | | |
| | YFYDCEPNKVNYLRSLGFRYTRLIDDITVSRLK | | |
| | KEGDWKFVETIISEFITQKELSVNKDKTQLLSAN | | |
| | SPQSFKVHGLCIEETTPRFTKNERINIKTQVKRV | | |
| | VKTGYNRDNIRMQKNYHDVYFSVKGKITKLKR | | |
| | VNCPDYPLLKKLLAKHCDPLPEHKEIKRINRVIS | | |
| | NLSKDHATFGSTERYRSRYFQVIFRLEILKKLYP | | |
| | VEANEFKARLKLISPIKNEN | | |
| 8258 | IYKGSKIDSLDKLSEVLSIDIDELTNVLLLEDEAK | WP_ | Acinetobacter |
| | YKAGFIKKSNGKLRNIYNPNTSLRKIQRRIKNRI | 005070670.1 | |
| | FTQQIEWPDYIFGSIPADEISSNDYVASAEKHCG | | |
| | ARALLKLDIEDFFDNITQELVEKIFKNFFKYNDE | | |
| | LSKILAQLCCVDGKVPQGGITSSYIASLALFSIEE | | |
| | RLFFRLKNKKLIYTRYIDDITISSKNSEYNFDSIIK | | |
| | IVEGQLNSIDLPLNIDKIKVERFSSKALKVHNIRV | | |
| | DLKTPKFDKIEVKNIRAAIH | | |
| 8259 | TQLNVDELASFVGTNAQTIQTITNKTTSYYKSFE | WP_ | Oenococcus oeni |
| | LKKRSGGSRTILAPKQQLLSIQKKIATTLEKIYPV | 032821736.1 | |
| | SIYSHGFIYKKGIKTNAEEHLRSTELLNFDIDNFF | | |
| | DNIPEYRIFGIFRYYFNMNNYISGILKELTCVERH | | |
| | LPQGAPSSPILSNIICYKIDKDLGKLARRNHCKY | | |
| | TRYVDDITFSSKRKLPSSIYNRINKSCSNNIVKIL | | |
| | SDSGFTINHRKTRLLTKSQRQEVTGITTNKQLNV | | |
| | SKTYIRSTRAMLY | | |
| 8260 | RWDESKKRRAENKKKREAENKQRREAWDIYR | OGQ87915.1 | Deltaproteobacteria |
| | KKTVVHAGEGVSSGLQDVLSDTDALVARGLPV | | bacterium |
| | MHCAADVAVMLGLPLPTLRWLTFHRRATALV | | |
| | HYHRFDIPKKTGGRRLISAPKATLKKAQQVVLD | | |
| | NILSRLPTEPEAHGFVAQHSIVTNAACHAGKAV | | |
| | VINVDLKDFFPSIGFRRVRGLFQRLGYSGQVAT | | |
| | MLGLLCTEPPRIKAELDGKVFHVALGERVLPQG | | |
| | ACTSPAITNTICRRLDRRLVGLASKHGFTYSRYA | | |
| | DDLTFSGNVPKKAGRLLRSVRAILENEGFAENG | | |
| | KKTRVMRQSRRQEVTGLTVNDKPRVSREQRRE | | |
| | LRAILH | | |
| 8261 | LVETFGSINNIKNALLDYFEYYECEKNKELVIMS | WP_ | Hungatella |
| | ILIRKELCTLDLASPFEYSIIDVIGSMSYFIEKLKT | 055655910.1 | hathewayi |
| | RVAFHQNYERLRYAPIKRWRARKRFSAYELFG | | |
| | MDIMEMRQMQSLFGYNKKKSVISKNLLINGKK | | |
| | RKIKMYSCTSEGFALRSFHLKLMKQLQKLIELQ | | |
| | PYSYAYRKDRSIFMCMNQHIDSKFFLKIDIKDFF | | |
| | NSISKGKMNKILKCHFCYDSKQAYEDNVIRRRS | | |
| | RYLGEYVKEWLGIKEITDICFVNGRLALGMVTS | | |
| | PILSNIYMDFFDERFHDNYPGLIYTRYSDDILISS | | |
| | GKWFDYKSILNFIAKELCYLELEINEHKVGFYK | | |
| | LKQAGDHIKFLGLNIVQGPEENYITVGKQYIKD | | |
| | VCSNIS | | |
| 8262 | YRSYDIKNIEEVKVRLLQAENYTKSIESSLKFNI | ABS14021.1 | Brucella anthropi |
| | AHTKGRALYFPQDYETEIIIRKANTNIKKILGINP | | ATCC 49188 |
| | VSRNDIIRHLKEILREGVPYVIGRYDIKRFYDNIK | | |
| | ISALNQNLDESLSTTYDTRRLVSGFLSSHEALYS | | |
| | SGLPTGISLSATLSELYLRNFDRGIKALPWVRYF | | |
| | ARYVDDIIIIAEPRTTAQLMESALISGLPDGLALN | | |
| | SGKDKRYFRKLERDFGGAGPEADFDYLGYRFK | | |
| | VDKILKKSSDCGTLASRKVTVDISEKKVKIRKTR | | |
| | FIYAVHKYLAD | | |
| 8263 | KGKKEKKKGAFFSSIEEVKKLKFDFVVSRISAHT | EMJ85443 | Leptospira |
| | KSDLLVEPTGYYDLEWFVKNRRSDLFNVIHSFY | | meyeri serovar |
| | SPSKKITLNMPKGNFSYRPVSYLLPMDSLIYLAV | | Semaranga str. |
| | TEKLIFYTKDKFSKFVYSNLLNPFDKKEVFSEPV | | Veldrot |
| | KHWLRMRNTIRSNYKTNSLDKYYSADISGYFE | | Semarang 173 |
| | NIKIKYLLKKAKFYIGKHEVSYTKYLKKLLEKW | | |
| | QYADSQGLIQPHPASSILGKIYLSPVDSYFSYLG | | |
| | KRYSRYVDEFHIQTDDLSEMLNITIHLNEQLREL | | |
| | GLNLNSKKTIFKIGKDIWDEINENQDFFSAVDYA | | |
| | QRIRKKDELA | | |
| 8264 | SGTPESKQSQPGGRKPPLMCHPAITYDAMCSLA | AF0732 | Turneriella parva |
| | GLQRAQISLIEELKRRGEGSKHALSYEDLTELGA | | DSM 21527 |
| | LLRSHQYSHRPCRLMTIKVGSKKRDIDSPDWLD | | |
| | RIVQRTYVDTIYPLVQQMACDSSHAYLYKRSIH | | |
| | TALWRLIMNIEHFGYSHVERTDIESFFDSIPHAE | | |
| | MERVIDLHIRDIELNAFSHELLRVAEGFKNSKVG | | |
| | LPTGWLIPPLWANMLLTPVDARLESAGLKFFRY | | |
| | GDDYGILQRSKQEAEFAQGLLESALKPLGLHLK | | |
| | PGYSHKTYTRKLEDGLIVLGHEIRRINNRLTVAI | | |
| | SKNSLAETR | | |
| 8265 | CIGDIESAAWRAFRGHSRKPEVRAFQESMSSNC | CCX61742.1 | Bacteroides sp. |
| | AFLYDSLRDGSWQNLMVYRQLTKTNNNGKVR | | CAG: 598 |
| | QIDSPSLVVRIYQHLLLNLLEPHYFRKDNLNGLN | | |
| | CKPGCGITSAIPSRSVIHRLKHIFYDRRDLHYCLT | | |
| | IDQRQCYDHITPKVFRKALKQMVDDKWLVDFA | | |
| | VDVCFVDGRLPIGTPTSPFVHHVVMLEFDYFVK | | |
| | SLSSASVRYADDNFLAFATKEEAQAAKWRIKN | | |
| | WWWFRLGMRAKRGSAVVRPLSEPCDFCGYVF | | |
| | HRVDGMGICDHNKGYVK | | |
| 8266 | RAARNNSAPGPNGVPYLVYKRCPKLLARLWKI | BAC82599.1 | Tetraodon |
| | LRVIWRRGKVAHQWRWAEGVWVPKEEKSTLI | | nigroviridis |
| | EQFRTISLLNVEGKIFFSILSHRLSDFLLKNQYIDS | | |
| | SVQKGGIPGVPGCLEHCGVVTQLIREAREGRGS | | |
| | LAVLWLDLANAYGAIPHKLVEMALARHHVPCS | | |
| | IKTLIMDYYDSFHLRFVTSGSVTSEWHRLEKGII | | |
| | TGCTISVIIFALAMNMLAKSAEPECRGPITKSGIR | | |
| | QPPIRAFMDDLTVTTTSVPGCRWILQGLERLMT | | |
| | WARMRFKPGKSRSLVLKAGKVTDRFRFYLGGT | | |
| | QIPSVSEKPVKSLGKMFDGSLKYAFS | | |
| 8267 | QVDWDEINEMLYERQWQKLGRGASTKTRIAQD | WP_0603863 | Bacteroides |
| | KTIIDLREMSVNGTRLNLDTALKNLRRDMIDDW | 31.1 | stercoris |
| | FFDPLQYVDLCNQDFVLDYFSSQDVREHYKFQE | | |
| | MEVLYIPKESLVQRKAMVGNFVDRLLYIAIVEK | | |
| | LAPLMEEYISSRVYAARLNRSEDNSLIANGVNQ | | |
| | WIKMNYLIDEWLEKGVGCLFKCDVVNYFDNIS | | |
| | HATLIGFLREIATDADALNAIKMLEQMFSEISDS | | |
| | QTNCGLPQNSDASSLLATFYLSHVDIQIQAQAIE | | |
| | YCRFMDDIYFMAPDYFSARNVLQSLEGELRRLN | | |
| | LCLNSSKVVCITLENKKEVDEFREGLSLYNHTN | | |
| | QKIKQLIR | | |
| 8268 | IAFIHPKNNEKANAHWEYYRDFLHDTDRFAESI | KJR43562 | Candidatus |
| | ATIPLDEFKLLYEKAYWHQKIEFPAIDYIWRTIQ | | Magnetoovum |
| | GASNDKIEYLEYSLNYAFQSWYSGKFIDKGLEY | | chiemensis |
| | SIPKQNPKKERRKVLLSPIDSIVEMKIIADIGPEIE | | |
| | NIIDAKFRQGGPVSLGNRLDLKENSLVKKRNLF | | |
| | KYWPKQFRLRYFTILQQIQDKTDGYIVNIDVSDF | | |
| | YPSIPHQEMMKIIEKYLPKLSNFQKHWINDFLKT | | |
| | GEMSKDDGKGIPQGPPLSHVLANLYLYDKLDS | | |
| | KIIHGFLPKFYTRYVDDIVYVASSKKDAETFYK | | |
| | WIDHVINPLVKNKDKSYIVSNSEYLNTYKQHEII | | |
| | ELIRIKVEQVIQKVYLIAKIL | | |
| 8269 | KEPDVSLGSTWLSDFPQAWAETGGMGLAVRQ | PE2d497 | |
| | APLIIPLKATSTPVSIKQYPMSQEARLGIKPHIQR | | |
| | LLDQGILVPCQSPWNTPLLPVKKPGTNDYRPVQ | | |
| | DLREVNKRVEDIHPTVPNPYNLLSGLPPSHQWY | | |
| | TVLDLKDAFFCLRLHPTSQPLFAFEWRDPEMGI | | |
| | SGQLTWTRLPQGFKNSPTLFNEALHRDLADFRI | | |
| | QHPDLILLQYVDDLLLAATSELDCQQGTRALLQ | | |
| | TLGNLGYRASAKKAQICQKQVKYLGYLLKEGQ | | |
| | RWLTEARKETVM | | |
| 8270 | TLNIEDEYRLHETSKEPDVSLGSTWLSDFPQAW | Q9YK99 | Murine leukemia |
| | AETGGMGLAVRQAPLIIPLKATSSPVSIKQYPMS | | virus |
| | QEARLGIKPHIQRLLDQGILVPCQSPWNTPLLPV | | |
| | KKPGTNDYRPVQDLREVNKRVEDIHPTVPNPY | | |
| | NLLSGLPPSHQWYTVLDLKDAFFCLRLHPTSQP | | |
| | LFAFEWRDPEMGISGQLTWTRLPQGFKNSPTLF | | |
| | DEALHRDLADFRIQHPDLILLQYVDDLLLAATS | | |
| | ENDCQQGTRALLQILGDLGYRASAKKAQICQK | | |
| | QVKYLGYLLKEGQRWLTEARKETVMGQPIPKT | | |
| | PRQLREFLGTAGFCRLWIPGFAEMAAPLYPLTK | | |
| | TGTLFNWGPDQQKAYQEIKQALLTAPALGLPD | | |
| | LTKPFELFVDEKQGYAKGVLTQRLGPWRRPVA | | |
| | YLSKKLDPVAAGWPPCLRMVAAIAVLTKDAGK | | |
| | LTMGQPLVVLAPHAVEALVKQPPDRWLSNAR | | |
| | MTHYQALLLDTDRVQFGPVVALNPATLLPLPEE | | |
| | GLRHDCLDILAEAHGTRPDLTDQPLPDADHTW | | |
| | YTDGSSFLQEGQRKAGAAVTTETEVIWAKALPS | | |
| | GTSAQRAELIALTQALKMAEGKKLNVYTDSRY | | |
| | AFATAHIHGEIYRRRGLLTSEGKEIKNKGEILAL | | |
| | LKALFLPKRLSIIHCPGHQKGNSAEARGNRMAD | | |
| | QAAREIASKETPETSTLLIENSTP | | |
| 8271 | TLNIEDEYRLHETSKEPDASLESTWLSDFPQAW | Q60FS9 | Murine leukemia |
| | AETGGMGLAVRQAPLIIPLKATSTPVSIKQYPMS | | virus |
| | QEARLGIKPHIQRLLDQGILVPCQSPWNTPLLPV | | |
| | KKPGTNDYRPVQDLREVNKRVEDIHPTVPNPY | | |
| | NLLSGLPPSHQWYTVLDLKDAFFCLRLHPTSQP | | |
| | LFAFEWRDPEMGISGQLTWTRLPQGFKNSPTLF | | |
| | DEALHRDLAGFRIQHPDLILLQYVDDLLLAATS | | |
| | ENDCQQGTRALLQILGDLGYRASAKKAQICQK | | |
| | QVKYLGYLLKEGQRWLTEARKETVMGQPIPKT | | |
| | PRQLREFLGTAGFCRLWIPGFAEMAAPLYPLTK | | |
| | TGTLFNWGPDQQKAYQEIKQALLTAPALGLPD | | |
| | LTKPFELFVDEKQGYAKGVLTQRLGPWRRPVA | | |
| | YLSKKLDPVAAGWPPCLRMVAAIAVLTKDAGK | | |
| | LTLGQPLVVLAPHAVEALVKQPPDRWLSNARM | | |
| | THYQALLLDTDRVQFGPVVALNPATLLPLPEEG | | |
| | LRHDCLDILAEAHGTRPDLTDQPLPDADHTWY | | |
| | TDGSSFLQKGQRKAGAAVTTETEVIWAKALPS | | |
| | GTSAQRAELIALTQALKMAEGKKLNVYTDSRY | | |
| | AFATAHIHGEIYRRRGLLTSEGKEIKNKGEILAL | | |
| | LKALFLPKRLSIIHCPGHQKGNSAEARGNRMAD | | |
| | QAAREIASKETPETSTLLIENSTP | | |
| 8272 | TLNIEDEYRLHETSKEPDVSLGSTWLSDFPQAW | P08361 | Cas-Br-E murine |
| | AETGGMGLAVRQAPLIIPLKATSTPVSIKQYPMS | | leukemia virus |
| | QEARLGIKPHIQRLLDQGILVPCQSPWNTPLLPV | | |
| | KKPGTNDYRPVQDLREVNKRVEDIHPTVPNPY | | |
| | NLLSGLPPSHQWYTVLDLKDAFFCLRLHPTSQP | | |
| | LFAFEWRDPEMGISGQLTWTRLPQGFKNSPTLF | | |
| | DEALHRDLAGFRIQHPDLILLQYVDDLLLAATS | | |
| | ELDCQQGTRALLQTLGDLGYRASAKKAQICQK | | |
| | QVKYLGYLLKEGQRWLTEARKETVMGQPIPKT | | |
| | PRQLREFLGTAGFCRLWIPGFAEMAAPLYPLTK | | |
| | TGTLFNWGPDQQKAFQEIKQALLTAPALGLPDL | | |
| | TKPFELFVDEKQGYAKGVLTQKLGPWRRPVAY | | |
| | LSKKLDPVAAGWPPCLRMVAAIAVLTKDAGKL | | |
| | TMGQPLVILAPHAVEALVKQPPDRWLSNARMT | | |
| | HYQALLLDTDRVQFGPVVALNPATLLPLPEEGL | | |
| | QHDCLDILAEAHGTRSDLMDQPLPDADHTWYT | | |
| | DGSSFLQEGQRKAGAAVTTETEVIWARALPAG | | |
| | TSAQRAELIALTQALKMAEGKKLNVYTDSRYA | | |
| | FATAHIHGEIYRRRGLLTSEGKEIKNKDEILALL | | |
| | KALFLPKRLSIIHCPGHQKGNSAEARGNRMADQ | | |
| | AAREVATRETPETSTLLIENSTP | | |
| 8273 | TLNIEDEYRLHETSKEPDVSLGSTWLSDFPQAW | O41250 | Rauscher murine |
| | AETGGMGLAVRQAPLIIPLKATSTPVSIKQYPIS | | leukemia virus |
| | QEARLGIKPHIQRLLDQGILVPCQSPWNTPLLPV | | |
| | KKPGTHDYRPVQDLREVNKRVEDIHPTVPNPY | | |
| | NLLSGLPPSHQWYTVLDLKDAFFCLRLHPTSQS | | |
| | LFAFEWRDPEMGISGQLTWTRLPQGFKNSPTLF | | |
| | DEALHRDLADFRIQHPDLILLQYVDDLLLAATS | | |
| | ELDCQQGTRALLQTLGDLGYRASAKKAQICQK | | |
| | QVKYLGYLLKEGQRWLTEARKETVMGQPTPKT | | |
| | PRQLREFLGTAGFCRLWIPGFAEMAAPLYPLTK | | |
| | TGTLFNWGPDQQKAYQEIKQALLTAPALGLPD | | |
| | LTKPFELFVDEKQGYAKGVLTQKLGPWRRPVA | | |
| | YLSKKLDPVAAGWPPCLRMVAAIAVLTKDAGK | | |
| | LTMGQPLVILAPHAVEALVKQPPDRWLSNARM | | |
| | THYQALLLDTDRVQFGPIVTLNPATLLPLPEEGL | | |
| | QHDCLDILAEAHGTRPDLTDQPLPDADHTWYT | | |
| | DGSSFLQEGQRKAGAAVTTETEVIWAKALPAG | | |
| | TSAQRAELIALTQALKMAEGKKLNVYTDSRYA | | |
| | FATAHIHGEIYRRRGLLTSEGKEIKNKDEILALL | | |
| | KALFLPKRLSIIHCPGHQKGNRAEARGNRMADQ | | |
| | AAREVATRETPETSTLLIENSTP | | |
| 8274 | TLNIEDEYRLHETSKGPDVPLGSTWLSDFPQAW | P26808 | Friend murine |
| | AETGGMGLAVRQAPLIIPLRAASTPVSIKQYPMS | | leukemia virus |
| | REARLGIKPHIQRLLDQGILVPCQSPWNTPLLPV | | (ISOLATE |
| | KKPGTNDYRPVQDLREVNKRVEDIHPTVPNPY | | PVC-211) |
| | NLLSGLPPSHQWYTVLDLKDAFFCLRLHPTSQS | | |
| | LFAFEWRDPEMGISGQLTWTRLPQGFKNSPTLF | | |
| | DEALHRDLADFRIQHPDLILLQYVDDLLLAATS | | |
| | ELDCQQGTRALLQTLGDLGYRASAKKAQICQK | | |
| | QVKYLGYLLKEGQRWLTEARKETVMGQPTPKT | | |
| | PRQLREFLGTAGFCRLWIPGFAEMAAPLYPLTK | | |
| | TGTLFKWGPDQQKAYQEIKQALLTAPALGLPD | | |
| | LTKPFELFVDEKQGYAKGVLTQKLGPWRRPVA | | |
| | YLSKKLDPVAAGWPPCLRMVAAIAVLTKDAGK | | |
| | LTMGQPLVILAPHAVEALVKQPPDRWLSNARM | | |
| | THYQALLLDTDRVQFGPIVTLNPATLLPLPEEGL | | |
| | QHDCLDILAEAHGTRPDLTDQPLPDADHTWYT | | |
| | DGSSFLQEGQRKAGAAVTTETEVIWAKALPAG | | |
| | TSAQRAELIALTQALKMAEGKKLNVYTDSRYA | | |
| | FATAHIHGEIYRRRGLLTSEGKEIKNKEEILALL | | |
| | KALFLPKRLSIIHCPGHQKGNRAEARGNRMADQ | | |
| | AAREVATRETPETSTLLIENSAP | | |
| 8275 | TLNIEDEYRLHETSKGPDVPLGSTWLSDFPQAW | P26809 | Friend murine |
| | AETGGMGLAVRQAPLIIPLKATSTPVSIKQYPMS | | leukemia virus |
| | QEARLGIKPHIQRLLDQGILVPCQSPWNTPLLPV | | (ISOLATE |
| | KKPGTNDYRPVQDLREVNKRVEDIHPTVPNPY | | FB29) |
| | NLLSGLPPSHQWYTVLDLKDAFFCLRLHPTSQS | | |
| | LFAFEWRDPEMGISGQLTWTRLPQGFKNSPTLF | | |
| | DEALHRDLADFRIQHPDLILLQYVDDLLLAATS | | |
| | ELDCQQGTRALLQTLGDLGYRASAKKAQICQK | | |
| | QVKYLGYLLKEGQRWLTEARKETVMGQPTPKT | | |
| | PRQLREFLGTAGFCRLWIPGFAEMAAPLYPLTK | | |
| | TGTLFEWGPDQQKAYQEIKQALLTAPALGLPDL | | |
| | TKPFELFVDEKQGYAKGVLTQKLGPWRRPVAY | | |
| | LSKKLDPVAAGWPPCLRMVAAIAVLTKDAGKL | | |
| | TMGQPLVILAPHAVEALVKQPPDRWLSNARMT | | |
| | HYQALLLDTDRVQFGPIVALNPATLLPLPEEGL | | |
| | QHDCLDILAEAHGTRPDLTDQPLPDADHTWYT | | |
| | DGSSFLQEGQRKAGAAVTTETEVVWAKALPAG | | |
| | TSAQRAELIALTQALKMAEGKKLNVYTDSRYA | | |
| | FATAHIHGEIYRRRGLLTSEGKEIKNKDEILALL | | |
| | KALFLPKRLSIIHCPGHQKGNRAEARGNRMADQ | | |
| | AAREVATRETPETSTLLIENSAP | | |
| 8276 | TLNIEDEYRLHETSKGPDVPLGSTWLSDFPQAW | P26810 | Friend murine |
| | AETGGMGLAFRQAPLIISLKATSTPVSIKQYPMS | | leukemia virus |
| | QEARLGIKPHIQRLLDQGILVPCQSPWNTPLLPV | | (ISOLATE 57) |
| | KKPGTNDYRPVQDLREVNKRVEDIHPTVPNPY | | |
| | NLLSGLPPSHQWYTVLDLKDAFFCLRLHPTSQS | | |
| | LFAFEWKDPEMGISGQLTWTRLPQGFKNSPTLF | | |
| | DEALHRDLADFRIQHPDLILLQYVDDLLLAATS | | |
| | ELDCQQGTRALLQTLGDLGYRASAKKAQICQK | | |
| | QVKYLGYLLKEGQRWLTEARKETVMGQPTPKT | | |
| | PRQLREFLGTAGLCRLWIPGFAEMAAPLYPLTK | | |
| | TGTLFKWGPDQQKAYQEIKQALLTAPALGLPD | | |
| | LTKPFELFVDEKQGYAKGVLTQKLGPWRRPVA | | |
| | YLSKKLDPVAAGWPPCLRMVAAIAVLTKDVGK | | |
| | LTMGQPLVILAPHAVEALVKQPPDRWLSNARM | | |
| | THYQALLLDTDRVQFGPIVALNPATLLPLPEEGL | | |
| | QHDCLDILAEAHGTRPDLTDQPLPDADHTWYT | | |
| | DGSSFLQEGQRRAGAAVTTETEVIWAKALPAG | | |
| | TSAQRAELIALTQALKMAAGKKLNVYTDSRYA | | |
| | FATAHIHGEIYRRRGLLTSEGKEIKNKDEILALL | | |
| | KALFLPKRLSIIHCPGHQKGNHAEARGNRMAD | | |
| | QAAREVATRETPETSTLLIENSAP | | |
| 8277 | TLNIEDEYRLHETSKEPDVPLGSTWLSDFPQAW | Q2F7J0 | Xenotropic |
| | AETGGMGLAVRQAPLIIPLKATSTPVSIKQYPMS | | MuLV-related |
| | QEARLGIKPHIQRLLDQGILVPCQSPWNTPLLPV | | virus VP42 |
| | KKPGTNDYRPVQDLREVNKRVEDIHPTVPNPY | | |
| | NLLSGLPPSHQWYTVLDLKDAFFCLRLHPTSQP | | |
| | LFAFEWRDPEMGISGQLTWTRLPQGFKNSPTLF | | |
| | DEALHRDLADFRIQHPDLILLQYVDDLLLAATS | | |
| | EQDCQRGTRALLQTLGNLGYRASAKKAQICQK | | |
| | QVKYLGYLLKEGQRWLTEARKETVMGQPTPKT | | |
| | PRQLREFLGTAGFCRLWIPGFAEMAAPLYPLTK | | |
| | TGTLFNWGPDQQKAYQEIKQALLTAPALGLPD | | |
| | LTKPFELFVDEKQGYAKGVLTQKLGPWRRPVA | | |
| | YLSKKLDPVAAGWPPCLRMVAAIAVLTKDAGK | | |
| | LTMGQPLVILAPHAVEALVKQPPDRWLSNARM | | |
| | THYQAMLLDTDRVQFGPVVALNPATLLPLPEK | | |
| | EAPHDCLEILAETHGTRPDLTDQPIPDADYTWY | | |
| | TDGGSFLQEGQRRAGAAVTTETEVIWGGVLPA | | |
| | GTSAQRAELIALTQALKMAEGKKLNVYTDSRY | | |
| | AFATAHVHGEIYRRRGLLTSEGREIKNKNEILAL | | |
| | LKALFLPKRLSIIHCPGHQKGNSAEARGNRMAD | | |
| | QAAREAAMKAVLETSTLLIEDSTP | | |
| 8278 | PHIQRLLDQGILVPCQSPWNTPLLPVKKPGTND | P03355 | Moloney murine |
| | YRPVQDLREVNKRVEDIHPTVPNPYNLLSGLPP | | leukemia virus |
| | SHQWYTVLDLKDAFFCLRLHPTSQPLFAFEWR | | isolate Shinnick |
| | DPEMGISGQLTWTRLPQGFKNSPTLFDEALHRD | | |
| | LADFRIQHPDLILLQYVDDLLLAATSELDCQQG | | |
| | TRALLQTLGNLGYRASAKKAQICQKQVKYLGY | | |
| | LLKEGQRWLTEARKETVMGQPTPKTPRQLREF | | |
| | LGTAGFCRLWIPGFAEMAAPLYPLTKTGTLFNW | | |
| | GPDQQKAYQEIKQALLTAPALGLPDLTKPFELF | | |
| | VDEKQGYAKGVLTQKLGPWRRPVAYLSKKLD | | |
| | PVAAGWPPCLRMVAAIAVLTKDAGKLTMGQPL | | |
| | VILAPHAVEALVKQPPDRWLSNARMTHYQALL | | |
| | LDTDRVQFGPVVALNPATLLPLPEEGLQHNCLD | | |
| | ILAEAHGTRPDLTDQPLPDADHTWYTDGSSLLQ | | |
| | EGQRKAGAAVTTETEVIWAKALPAGTSAQRAE | | |
| | LIALTQALKMAEGKKLNVYTDSRYAFATAHIH | | |
| | GEIYRRRGLLTSEGKEIKNKDEILALLKALFLPK | | |
| | RLSIIHCPGHQKGHSAEARGNRMADQAARKAAI | | |
| | TE | | |
| 8279 | TLNLEDEYRLYETSAEPEASPGSTWLSDFPQAW | Q9WHV7 | Murine leukemia |
| | AETGGMGLAVRRRPLIIPLNATSTPVSIKQYPMS | | virus |
| | QEARLGIKPHIQRLLDQGILVPCQSPWNTPCLPV | | |
| | KKPGTNDYRPVQDLREVNKRVEDIHPTVPNPY | | |
| | NLLSGLPPSHRWYTVLDLKDAFFCLRLHPTSQP | | |
| | LFAFEWRDPGMGISGQLTWTRLPQGFKNSPTLF | | |
| | DEALHRDLADFRIQHPDLILLQYVDDILLAATSE | | |
| | LDCQQGTRALLLTLGNLGYRASAKKAQLCQKQ | | |
| | VKYLGYLLREGQRCLTEARKETVRGQPTPKTPR | | |
| | QLREFLGTAGFCRLWIPGFAEMAAPLYPLTKTG | | |
| | TLFNWGPDQQKAYQEIKQALLTAPALGLPDLT | | |
| | KPFELFVDEKQGYAKGSLTQKLGPWRRPVAYL | | |
| | SKKLDPVAAGWPPCLRMVAAIAVLRKDAGKLT | | |
| | MGQPLVILAPHADEALVKQPPDRWLSNARMTH | | |
| | YQAMLLDTDRVQFGPVVALNPSTFIPLPEEGAP | | |
| | HDCLEILAETHGTRPDLTDQPIPDADHTWYTDG | | |
| | SSFLQEGQRKAGAAVTTETEVIWARALPAGTSA | | |
| | QRELIALTQALKMAEGKRLNVYTDSRYAFATA | | |
| | HIHGEIYRRRGLLTSEGREIKNKSEILALLKALFL | | |
| | PKRLSIIHCLGHQKGDSAEARGNRLADQAAREA | | |
| | AINTPPDTSTLLIEDSTP | | |
| 8280 | TLNIEDEYRLHEISTEPDVSPGSTWLSDFPQAWA | P11227 | Radiation murine |
| | ETGGMGLAVRQAPLIIPLKATSTPVSIKQYPMSQ | | leukemia virus |
| | EAKLGIKPHIQRLLDQGILVPCQSPWNTPLLPVK | | |
| | KPGTNDYRPVQGLREVNKRVEDIHPTVPNPYNL | | |
| | LSGLPTSHRWYTVLDLKDAFFCLRLHPTSQPLF | | |
| | ASEWRDPGMGISGQLTWTRLPQGFKNSPTLFDE | | |
| | ALHRGLADFRIQHPDLILLQYVDDLLLAATSEL | | |
| | DCQQGTRALLKTLGNLGYRASAKKAQICQKQV | | |
| | KYLGYLLREGQRWLTEARKETVMGQPTPKTPR | | |
| | QLREFLGTAGFCRLWIPRFAEMAAPLYPLTKTG | | |
| | TLFNWGPDQQKAYHEIKQALLTAPALGLPDLT | | |
| | KPFELFVDEKQGYAKGVLTQKLGPWRRPVAYL | | |
| | SKKLDPVAAGWPPCLRMVAAIAVLTKDAGKLT | | |
| | MGQPLVILAPHAVEALVKQPPDRWLSNARMTH | | |
| | YQAMLLDTDRVQFGPVVALNPATLLPLPEEGAP | | |
| | HDCLEILAETHGTEPDLTDQPIPDADHTWYTDG | | |
| | SSFLQEGQRKAGAAVTTETEVIWARALPAGTSA | | |
| | QRAELIALTQALKMAEGKRLNVYTDSRYAFAT | | |
| | AHIHGEIYKRRGLLTSEGREIKNKSEILALLKALF | | |
| | LPKRLSIIHCLGHQKGDSAEARGNRLADQAARE | | |
| | AAIKTPPDTSTLLIEDSTP | | |
| 8281 | TLGIEDEYRLHETSTEPDVSLGSTWLSDFPQAW | Q7SVK7 | Murine leukemia |
| | AETGGMGLAVRQAPLIIPLKATSTPVSIQQYPMS | | virus (strain |
| | HEARLGIKPHIQRLLDQGILVPCQSPWNTPLLPV | | BM5 ECO) |
| | KKPGTNDYRPVQDLREVNKRVEDIHPTVPNPY | | |
| | NLLSGLPPSHQWYTVLDLKDAFFCLRLHPTSQP | | |
| | LFAFEWRDPGMGISGQLTWTRLPQGFKNSPTLF | | |
| | DEALHRDLADFRIQHPDLILLQYVDDILLAATSE | | |
| | LDCQQGTRALLQTLGDLGYRASAKKAQICQKQ | | |
| | VKYLGYLLREGQRWLTEARKETVMGQPVPKTP | | |
| | RQLREFLGTAGFCRLWIPGFAEMAAPLYPLTKT | | |
| | GTLFSWGPDQQKAYQEIKQALLTAPALGLPDLT | | |
| | KPFELFVDEKQGYAKGVLTQKLGPWRRPVAYL | | |
| | SKKLDPVAAGWPPCLRMVAAIAVLTKDAGKLT | | |
| | MGQPLVILAPHAVEALVKQPPDRWLSNARMTH | | |
| | YQAMLLDTDRVQFGPVVALNPATLLPLPEEGAP | | |
| | HDCLEILAETHGTRPDLTDQPIPDADHTWYTDG | | |
| | SSFLQEGQRKAGAAVTTETEVIWAGALPAGTSA | | |
| | QRAELIALTQALKMAEGKRLNVYTDSRYAFAT | | |
| | AHIHGEIYRRRGLLTSEGREIKNKSEILALLKALF | | |
| | LPKRLSIIHCLGHQKGDSAEARGNRLADQAARE | | |
| | AAIKTPPDTSTLLIEDSTP | | |
| 8282 | TLNLEDEYRLYETSAEPEASPGSTWLSDFPQAW | Q90RL4 | Murine leukemia |
| | AETGGMGLAVRQAPLIIPLKATSTPVSIKQYPMS | | virus |
| | QEARLGIKPHIQRLLDQGILVPCQSPWNTPLLPV | | |
| | KKPGTNDYRPVQDLREVNKRVEDIHPTVPNPY | | |
| | NLLSGLPPSHRWYTVLDLKDAFFCLRLHPTSQP | | |
| | LFAFEWRDPGMGISGQLTWTRLPQGFKNSPTLF | | |
| | DEALHRDLAGFRIQHPDLILLQYVDDLLLAATS | | |
| | ELDCQQGTRALLQTLGDLGYRASAKKAQICQK | | |
| | QVKYLGYLLKEGQRWLTEARKETVMGQPIPKT | | |
| | PRQLREFLGTAGFCRLWIPGFAEMAAPLYPLTK | | |
| | TGTLFNWGPDQQKAYQEIKQALLTAPALGLPD | | |
| | LTKPFELFVDEKQGYAKGVLTQKLGPWRRSVA | | |
| | YLSKKLDPVAAGWPPCLRMVAAIAVLTKDAGK | | |
| | LTMGQPLVILAPHAEEALVKQPPDRWLSNARM | | |
| | THYQAMLLDTDRVQFGPVVALNPATLLPLPEE | | |
| | GAPHDCLEILAETHGTRPDLTDQPIPDADHTWY | | |
| | SDGSSFLQEGQRKAGAAVTTETEVIWARALPAG | | |
| | TSAQRAELIALTQALKMAEGKRLNVYTDSRYA | | |
| | FATAHIHGEIYRRRGLLTSEGREIKNKSEILALLK | | |
| | ALFLPKRLSIIHCLGHQKGDSAEARGNRLADQA | | |
| | AREAAIKTPPDTSTLLIEDSTP | | |
| 8283 | TLNLEDEYRLYETSAEPEVSPGSTWLSDFPQAW | P03356 | AKR |
| | AETGGMGLAVRQAPLIIPLKATSTPVSIKQYPMS | | (endogenous) |
| | QEAKLGIKPHIQRLLDQGILVPCQSPWNTPLLPV | | murine leukemia |
| | KKPGTNDYRPVQDLREVNKRVEDIHPTVPNPY | | virus |
| | NLLSGLPPSHRWYTVLDLKDAFFCLRLHPTSQP | | |
| | LFAFEWRDPGMGISGQLTWTRLPQGFKNSPTLF | | |
| | DEALHRDLADFRIQHPDLILLQYVDDILLAATSE | | |
| | LDCQQGTRALLLTLGNLGYRASAKKAQLCQKQ | | |
| | VKYLGYLLKEGQRWLTEARKETVMGQPTPKTP | | |
| | RQLREFLGTAGFCRLWIPGFAEMAAPLYPLTKT | | |
| | GTLFNWGPDQQKAYQEIKQALLTAPALGLPDL | | |
| | TKPFELFVDEKQGYAKGVLTQKLGPWRRPVAY | | |
| | LSKKLDPVAAGWPPCLRMVAAIAVLRKDAGKL | | |
| | TMGQPLVILAPHAVEALVKQPPDRWLSNARMT | | |
| | HYQAMLLDTDRVQFGPVVALNPATLLPLPEEG | | |
| | APHDCLEILAETHGTRPDLTDQPIPDADHTWYT | | |
| | DGSSFLQEGQRKAGAAVTTETEVIWARALPAG | | |
| | TSAQRAELIALTQALKMAEGKRLNVYTDSRYA | | |
| | FATAHIHGEIYRRRGLLTSEGREIKNKSEILALLK | | |
| | ALFLPKRLSIIHCLGHQKGDSAEARGNRLADQA | | |
| | AREAAIKTPPDTSTLLIEDSTP | | |
| 8284 | TLQLEDEYRLYEPEQDKPKSPEIDSWVTKFPLA | Q7ZKZ7 | Recombinant M- |
| | WAETGGMGLALQQPPLIIQLKATATPVSIKQYP | | MuLV/RaLV |
| | MSWEAYQGIKPHIRRLLDQGILVPCRSPWNTPL | | retrovirus |
| | LPVKKPGTGDYRPVQDLREVNKRVEDIHPTVPN | | |
| | PYNLLSTLQTTHTWYTVLDLKDAFFCLRLSPES | | |
| | QPLFAFEWKDSEMGLSGQLTWTRLPQGFKNSP | | |
| | TLFDEALHRDLADFRVQHPTLILLQFVDDLLLG | | |
| | ATSETACHQGTESLLQTLGRLGYRASARKAQIC | | |
| | QTQVTYLGYQLRDGQRWLTPARKQTVANIPAP | | |
| | RNGRQLREFLGTAGFCRLWIPGFAEMAAPLYPL | | |
| | TKQGVLFQWGAEQQEAFDNIKRALLSSPALGLP | | |
| | DITKPFELFVDEKQGYAKGVLTQRLGPWKRPV | | |
| | AYLSKKLDPVASGWPPCLRMVAAIAVLTKDAG | | |
| | KLTLGQPLTILAPHAVEALIKQPPDCWLSNSRM | | |
| | THYQALLLDAERVQFGPVVALNPATLLPLPEEA | | |
| | EQHDCLQILAEVHGTRPDLSDRPLQDADHTWY | | |
| | TDGSSYLVNGERKAGAAVTTEDKVIWASALPV | | |
| | GTSAQRAELIALTQALKMAEGKRLNVYTDSRY | | |
| | AFATAHIHGEIYRRRGLLTSEGKDIKNKTEILAL | | |
| | LAALFLPKRLSIIHCPGHQKGHSPEARGNRLAD | | |
| | VSAREAAMGTQVLSLKDQDQPTSP | | |
| 8285 | TLQLEDEYRLYEPEQDKPKSLEIDSWATKFPLA | Q7ZKZ9 | Recombinant M- |
| | WAETGGMGLALQQPSLIIQLKSTATPSSIKQYP | | MuLV/RaLV |
| | MSWEAYQGIKPHIRRLLDQGILVPCRSPWNMPL | | retrovirus |
| | LPVKKPGTGDYRPVQDLREVNKRVEDIHPTVPN | | |
| | PYNLLSTLPPTHTWYTVLDLKDAIFCLRLSPESQ | | |
| | PLFAFEWKDSEMGLSGQLTWTRLPQGFKNSPM | | |
| | LFDEALHRDLADFRVQHPTLILLQFVDDLLLGA | | |
| | TSETACHQGTESLLQTLGRLGYRASARKAQICQ | | |
| | TQVTYLGYQLRDGQRWLTPARKQTVANIPAPR | | |
| | NGRQLREFLGTAGFCRLWIPGFAEMAAPLYPLT | | |
| | KQGVLFQWGAEQQEAFDNIKRALLSSSALGLPE | | |
| | ITKPFELFVDEKQGYAKGVLTQRLGPWNHPVA | | |
| | YLSKKLDPVASGWPPCLRMVAAIAVLTKDAGK | | |
| | LTLGQPLTILAPHAVEALIKQPPGRWLSNSRMT | | |
| | HYQALLLDAEWVQFGPVVALNPATLLPLTEEA | | |
| | EQHDCLQILAEVHGIRPDLSDRPLQDADHTWYT | | |
| | DGSSYLVNGERKAGAAVTTEDKVIWASALPVG | | |
| | TSAQRAELIALTQALKMAEGKRLNVYTDRHYA | | |
| | FATAHIHGEIYQRRGLLTSEGKDIKNKTEIQALL | | |
| | AALFLPKRLRIIHCPGHQKGHSPEARGNRLADV | | |
| | SAPEAAMGTQVLFLKDQDQPTSP | | |
| 8286 | PLQLEDEYRLYEPEQAKPKSLEIDSWVTKFPLA | Q7ZKZ5 | Recombinant M- |
| | WAETGGMGLALQQPPLIIQLKATATPVSIKQYP | | MuLV/RaLV |
| | MSWEAYQGIKPHIRRLLDQGILVPCWSPWNTPL | | retrovirus |
| | LPVKKPGTGDYRPVQDLREVNKRVEDIHPAVP | | |
| | NPYNLLSTLPPTHTWYMVLDLKDAFFCLRLSPE | | |
| | SQPLFAFEWKDSEMGLSGQLTWTRLPQGFKNSP | | |
| | TLFDEALHRDLADFRVQHPTLILLQFVDDLLLG | | |
| | ATSETACHQGTESLLQTLGRLGYRASARKAQIC | | |
| | QTQVTYLGYQLRDGQRWLTPARKQTVANIPAP | | |
| | RNGRQLREFLGTAGFCRLWIPGFAEMAAPLYPL | | |
| | TKQGVLFQWGAEQQEAFDNIKRALLSSPALGLP | | |
| | DITKPFELFVDEKQGCAKGVLTQRLGPWKCPV | | |
| | VYLSKKLDPVASGCSPCLRMVAAIAVLTKDAG | | |
| | KLTLGQPLTILAPHAVEALIKQPPDRWLSNSRM | | |
| | THYQALLLDAEWVQFGPVVALNPATLLPLTEE | | |
| | AEQHDCLQILAEVHGIRPDLSDRPLQDADHTWY | | |
| | TDGSSYLVNGERKAGAAVTTEDKVIWASALPV | | |
| | GTSAQRAELIALTQALKMAEGKRLNVYTDRHY | | |
| | AFATAHIHGEIYQRRGLLTSEGKDIKNKTEIQAL | | |
| | LAALFLPKRLRIIHCPGHQKGHSPEARGNRLAD | | |
| | VSAPEAAMGTQVLFLKDQDQPTSP | | |
| 8287 | TLQLEEEYRLFEPESTQKQEMDIWLKNFPQAWA | P10273 | Feline leukemia |
| | ETGGMGTAHCQAPVLIQLKATATPISIRQYPMP | | virus |
| | HEAYQGIKPHIRRMLDQGILKPCQSPWNTPLLP | | |
| | VKKPGTEDYRPVQDLREVNKRVEDIHPTVPNPY | | |
| | NLLSTLPPSHPWYTVLDLKDAFFCLRLHSESQLL | | |
| | FAFEWRDPEIGLSGQLTWTRLPQGFKNSPTLFD | | |
| | EALHSDLADFRVRYPALVLLQYVDDLLLAAAT | | |
| | RTECLEGTKALLETLGNKGYRASAKKAQICLQE | | |
| | VTYLGYSLKDGQRWLTKARKEAILSIPVPKNSR | | |
| | QVREFLGTAGYCRLWIPGFAELAAPLYPLTRPG | | |
| | TLFQWGTEQQLAFEDIKKALLSSPALGLPDITKP | | |
| | FELFIDENSGFAKGVLVQKLGPWKRPVAYLSKK | | |
| | LDTVASGWPPCLRMVAAIAILVKDAGKLTLGQ | | |
| | PLTILTSHPVEALVRQPPNKWLSNARMTHYQA | | |
| | MLLDAERVHFGPTVSLNPATLLPLPSGGNHHDC | | |
| | LQILAETHGTRPDLTDQPLPDADLTWYTDGSSFI | | |
| | RNGEREAGAAVTTESEVIWAAPLPPGTSAQRAE | | |
| | LIALTQALKMAEGKKLTVYTDSRYAFATTHVH | | |
| | GEIYRRRGLLTSEGKEIKNKNEILALLEALFLPK | | |
| | RLSIIHCPGHQKGDSPQAKGNRLADDTAKKAAT | | |
| | ETHSSLTVLPTELIEG | | |
| 8288 | TVSLQDEHRLFDIPVTTSLPDVWLQDFPQAWAE | P10272 | Baboon |
| | TGGLGRAKCQAPIIIDLKPTAVPVSIKQYPMSLE | | endogenous |
| | AHMGIRQHIIKFLELGVLRPCRSPWNTPLLPVKK | | virus strain M7 |
| | PGTQDYRPVQDLREINKRTVDIHPTVPNPYNLLS | | |
| | TLKPDYSWYTVLDLKDAFFCLPLAPQSQELFAF | | |
| | EWKDPERGISGQLTWTRLPQGFKNSPTLFDEAL | | |
| | HRDLTDFRTQHPEVTLLQYVDDLLLAAPTKKA | | |
| | CTQGTRHLLQELGEKGYRASAKKAQICQTKVT | | |
| | YLGYILSEGKRWLTPGRIETVARIPPPRNPREVR | | |
| | EFLGTAGFCRLWIPGFAELAAPLYALTKESTPFT | | |
| | WQTEHQLAFEALKKALLSAPALGLPDTSKPFTL | | |
| | FLDERQGIAKGVLTQKLGPWKRPVAYLSKKLD | | |
| | PVAAGWPPCLRIMAATAMLVKDSAKLTLGQPL | | |
| | TVITPHTLEAIVRQPPDRWITNARLTHYQALLLD | | |
| | TDRVQFGPPVTLNPATLLPVPENQPSPHDCRQV | | |
| | LAETHGTREDLKDQELPDADHTWYTDGSSYLD | | |
| | SGTRRAGAAVVDGHNTIWAQSLPPGTSAQKAE | | |
| | LIALTKALELSKGKKANIYTDSRYAFATAHTHG | | |
| | SIYERRGLLTSEGKEIKNKAEIIALLKALFLPQEV | | |
| | AIIHCPGHQKGQDPVAVGNRQADRVARQAAM | | |
| | AEVLTLATEPDNTSH | | |
| 8289 | LQDFPQAWAETGGLGRAKCQVPIIIDLKPTAMP | P31792 | Feline |
| | VSIRQYPMSKEAHMGIQPHITRFLELGVLRPCRS | | endogenous |
| | PWNTPLLPVKKPGTRDYRPVQDLREVNKRTMD | | virus ECE1 |
| | IHPTVPNPYNLLSTLSPDRTWYTVLDLKDAFFCL | | |
| | PLAPQSQELFAFEWRDPERGISGQLTWTRLPQG | | |
| | FKNSPTLFDEALHRDLTDFRTQHPEVTLLQYVD | | |
| | DLLLAAPTKEACIRGTKHLLRELGDKGYRASAK | | |
| | KAQICQTKVTYLGYILSEGKRWLTPGRIETVAHI | | |
| | PPPQNPREVREFLGTAGFCRLWIPGFAELAAPLY | | |
| | ALTKESAPFTWQEKHQSAFEALKEALLSAPALG | | |
| | LPDTSKPFTLFIDEKQGIAKGVLTQKLGPWKRP | | |
| | VAYLSKKLDPVAAGWPPCLRIMAATAMLVKDS | | |
| | AKLTLGQPLTVITPHALEAIVRQTPDRWITNARL | | |
| | THYQALLLDTDRIQFGPPVTLNPATLLPAPEDQ | | |
| | QSAHDCRQVLAETHGTREDLKDQELPDADHSW | | |
| | YTDGSSYIDSGTRRAGAAVVDGHHIIWAQSLPP | | |
| | GTSAQKAELIALTKALELSEGKKANIYTDSRYA | | |
| | FATAHTHGSIYERRGLLTSEGKEIKNKAEIIALL | | |
| | KALFLPRKVAIIHCPGHQKGQDPIATGNRQADQ | | |
| | VARQVAVAETLTLTTKLEETNL | | |
| | TLQLDDEYRLFSPPVKLDQNIQFGSTQFPQALAE | | |
| 8290 | PAGMGLAKQVPPQVIQLKPSLAPVPVRQSPFSK | Q8Q6U4 | Porcine |
| | EAREGIRPHVQRLIQQGIIVPVQSPWNTPLLPVR | | endogenous |
| | KPGTNDYRPVQDFERGQKRVQDIHPTVPNPYNL | | retrovirus |
| | LCALPPQRSWYTVLDLKDAFFCLRLHPTSQPLF | | |
| | AFEWRDPGAGRTGQLTWTRLPQGFKNFPTIFDQ | | |
| | ALHRDLANFRIQHPQVTLLQYVDDLVLAAATK | | |
| | QDCLQRPKGLLVELSDLGDRAFGYKAHICPTEV | | |
| | TYLGYRLRGRHRWLTEAPQTTVVQIPGPTPAKQ | | |
| | VREFLGTVGFCRLWIPGFATLPAPLYPLPKEKGE | | |
| | FSWALQHQKAFDAIKKALLSAPALALPDVTKTL | | |
| | YVDERKGVARGVLTQTLGPWRRPVAYLSKKLD | | |
| | PVASGWPICLKAIAAVAILVKDADKLTLGQNIT | | |
| | VIAPHALENIVRQPPDRWMTNARMTHYQSLLL | | |
| | TERVTFAPPAALNPATLLPEETDEPLTHDCHQLL | | |
| | IEETGVRKDLTDIPLTGEPVTWFTDGSSYLVEGN | | |
| | KMAGAAVVDRTPTIWGTNLPERTSSQKGELIGL | | |
| | MQAFRLGQGKSINIYTDSRYAFATAHVHGAIYT | | |
| | QRGLLTSAGREIKNKEEILSLLEALHLPKRLAIIH | | |
| | CPGHQKAKDPISRGNQMADRVAKQAAQGVNL | | |
| | LPMIETPKAP | | |
| 8291 | TLQLDEYRLYSPLVKPDQNIQFWLEQFPKAWAE | Q8Q6U7 | Porcine |
| | TAGMGLAKQVPPQVIQLKASAAPVSVRQYLLS | | endogenous |
| | KEAREGIGPHVQRLIQQGILVPVQSPWNTPLLPV | | retrovirus |
| | RKPGTNDYRPVQDLREVNKRVQDIHPTVPNPY | | |
| | NLLCALPPQRSWYTVLDLKDAFFCLRLHPTSQP | | |
| | LFAFEWRDPGAGRTGQLTWTRLPQGFKNSPTIF | | |
| | DEALHRDLANFRIQHPQVTLLQYVDDLLLAGA | | |
| | TKQDCLEGLLLELFDLGYRASAKKAQICRREAT | | |
| | QLGVQVCGAGQSDWLTGKARKKTVQPKIGPPT | | |
| | TAKQVVREFFGAQVGFCRLWIPGFATLAAPLYP | | |
| | LTKEKGEFSWALEHQKAFDAIKKALSSAPALAL | | |
| | PDVLKPFTLYVDERKGVARGVLTQILGPWRRPV | | |
| | AYLSKKLDPVASGWPICLKAIAAVAILVKDADK | | |
| | LTLGQNITVIAPHALENIVRQPPDRWMTNARMT | | |
| | HYQSLLLTERVTFAPPAALNPATLLPEETDEPVT | | |
| | HDCHLLIEETGVRKDLTDIPPLTGKMLTWFTDG | | |
| | SSYVVEGKSMAGPPVVTGTRTIWASSLPEGTSA | | |
| | QKAELMALTQALRLAEGKSINIYTDSRYAFATA | | |
| | HVHGAIYKQRGLLTSAGREIKTKEEILSLLEALH | | |
| | LPKRLAIIHCPGHQKAKDPISRGNQMADRVAKQ | | |
| | AAQGVNLLPMIETPKAP | | |
| 8292 | TLQLDDEYRLYSPLVKPDQNIQSWLEQFPQAW | ADK35878.1 | Porcine |
| | AETAGMGLAKQVPPQVIQLKASATPISVRQYPL | | endogenous |
| | SREAREEIWPHVQRLIQQGILVPVRSPWNTPLLP | | retrovirus C |
| | VRKPGTNDYRPVQDLREVNKRVQDIHPMVPNP | | |
| | YNLLSALPPKRNWYTVLDLKNAFFCLRLHPTSQ | | |
| | PLFAFEWRDPGTGRTGQLTWTRLPQGFKNSPTI | | |
| | FDEALHRDLANFRIQHPQVTLLQYVDDLLLAGA | | |
| | TKQDCLEGTNALLLELSDLGYRASAKKAQICRR | | |
| | EVTYLGYSLRDGQRWLTEARKRTVVQILAPTT | | |
| | AKQVREFLGTAGFCRLWIPGFATLAAPLYPLTK | | |
| | EKGEFSWAPEHQKAFDAIKKALLSAPALALPDV | | |
| | TKPFTLYVDEHKGVARGVLTQSLGPWRRPVAY | | |
| | LSKKLDPVASGWPVCLKAIAAVAILVKDADKST | | |
| | LGQNITVIAPHALENIVRQPPDRWMTNARMTH | | |
| | YQSLLLTERITFAPPAALNPATLLPEETDEPVTH | | |
| | DCHQLLIEETGVRKDLTDIPLTGEVLTWFTDGSS | | |
| | YVVEGKRMARAAVVDGTRTIWASSLSEGTSAQ | | |
| | KAELVALTQALRLAEGKSINIYTDSRYAFATAH | | |
| | VHGAIYKQRGLLTSAGREVKNKEKILSLLEALH | | |
| | LPKRLAIIHCPGHQKAKDLISRGNQMADRVAKQ | | |
| | AAQGVNLLPIIETPKAP | | |
| 8293 | TLQLDDEYRLYSPLVKPDQNIQFWLEQFPQAW | Q5QGQ8 | Porcine |
| | AETAGMGLAKQVPPQVIQLKASATPVSVRQYP | | endogenous |
| | LSKEAQEGIRPHVQRLIQQGILVPVQSPWNTPLL | | retrovirus C/A |
| | PVRKPGTNDYRPVQDLREVNKRVQDIHPTVPNP | | |
| | YNLLCALPPQRSWYTVLDLKDAFFCLRLHPTSQ | | |
| | PLFAFEWRDPGTGRTGQLTWTRLPQGFKNSPTI | | |
| | FDEALHRDLANFRIQHPQVTLLQYVDDLLLAGA | | |
| | TKQDCLEGTKALLLELSDLGYRASAKKAQICRR | | |
| | EVTYLGYSLRDGQRWLTEARKKTVVQIPAPTT | | |
| | AKQMREFLGTAGFCRLWIPGFATLAAPLYPLTK | | |
| | EKGEFSWAPEHQKAFDAIKKALLSAPALALPDV | | |
| | TKPFTLYVDERKGVARGVLTQTLGPWRRPVAY | | |
| | LSKKLDPVASGWPICLKAIAAVAILVKDADKLT | | |
| | LGQNITVIAPHALENIVRQPPDRWMTNARMTH | | |
| | YQSLLLTERVTFAPPAALNPATLLPEETDEPVTH | | |
| | DCHQLLIEETGVRKDLTDIPLTGEVLTWFTDGSS | | |
| | YVVEGKRMAGAAVVDGTRTIWASSLPEGTSAQ | | |
| | KAELMALTQALRLAEGKSINIYTDSRYAFATAH | | |
| | VHGAIYKQRGLLTSAGREIKNKEEILSLLEAVHL | | |
| | PKRLAIIHCPGHQKAKDLISRGNQMADRVAKQ | | |
| | AAQGVNLLPIIEMPKAP | | |
| 8294 | TLQLDDEYRLYSPLVKPDQNIQFWLEQFPQAW | Q4VFZ2 | Porcine |
| | AETAGMGLAKQVPPQVIQLKASATPVSVRQYP | | endogenous |
| | LSKEAQEGIRPHVQRLIQQGILVPVQSPWNTPLL | | retrovirus C/A |
| | PVRKPGTNDYRPVQDLREVNKRVQDIHPTVPNP | | |
| | YNLLCALPPQRSWYTVLDLKDAFFCLRLHPTSQ | | |
| | PLFAFEWRDPGTGRTGQLTWTRLPQGFKNSPTI | | |
| | FDEALHRDLANFRIQHPQVTLLQYVDDLLLAGA | | |
| | TKQDCLEGTKALLLELSDLGYRASAKKAQICRR | | |
| | EVTYLGYSLRDGQRWLTEARKKTVVQIPAPTT | | |
| | AKQVREFLGTAGFCRLWIPGFATLAAPLYPLTK | | |
| | EKGEFSWAPEHQKAFDAIKKALLSAPALALPDV | | |
| | TKPFTLYVDERKGVARGVLTQTLGPWRRPVAY | | |
| | LSKKLDPVASGWPVCLKAIAAVAILVKDADKL | | |
| | TLGQNITVIAPHALENIVRQPPDRWMTNARMTH | | |
| | YQSLLLTERVTFAPPAALNPATLLPEETDEPVTH | | |
| | DCHQLLIEETGVRKDLTDIPLTGEVLTWFTDGSS | | |
| | YVVEGKRMAGAAVVDGTRTIWASSLPEGTSAQ | | |
| | KAELMALTQALRLAEGKSINIYTDSRYAFATAH | | |
| | VHGAIYKQRGLLTSAGREIKNKEEILSLLEALHL | | |
| | PKRLAIIHCPGHQKAKDPISRGNQMADRVAKQA | | |
| | AQGVNLLPMIETPKAP | | |
| 8295 | TLQLDDEYRLYSPLVKPDQNIQFWLEQFPQAW | Q90RL9 | Porcine |
| | AETAGMGLAKQVPPQVIQLKASAAPVSVRQYP | | endogenous |
| | LSKEAREGIRPHVQRLIQQGILVPVQSPWNTPLL | | retrovirus C |
| | PVRKPGTNDYRPVQDLREVNKRVQDIHPTVPNP | | |
| | YNLLCALPPQRSWYTVLDLKDAFFCLRLHPTSQ | | |
| | PLFAFEWRDPGAGRTGQLTWTRLPQGFKNSPTI | | |
| | FDEALHRDLANFRIQHPQVTLLQYVDDLLLAGA | | |
| | TKQDCLEGTKALLLELSDLGYRASAKKAQICRR | | |
| | EVTYLGYSLRGGQRWLTEARKRTVVQIPAPTTA | | |
| | KQVREFLGTAGFCRLWIPGFATLAAPLYPLTKE | | |
| | KGEFSWAPEHQKAFDAIKKALLSAPALALPDVT | | |
| | KPFTLYVDERKGVARGVLTQTLGPWRRPVAYL | | |
| | SKKLDPVASGWPICLKAIAAVAILVKDADKLTL | | |
| | GQNITVIAPHALENIVRQPPDRWMTNARMTHY | | |
| | QSLLLTERVTFAPPAALNPATLLPEETDEPVTHD | | |
| | CHQLLIEETGVRKDLTDIPLTGEMLTWFTDGSS | | |
| | YMVEGKRMAGAAVVDGTRTIWASSLPEGTSAQ | | |
| | KAELMALTQALRLAEGKSINIYTDSRYAFATAH | | |
| | VHGAIYKQRGLLTSAGREIKNKEEILSLLEALHL | | |
| | PKRLAIIHCPGHQKAKDPISRGNQMADRVAKQA | | |
| | AQGVNLLPMIETPKAP | | |
| 8296 | TLQLDDEYRLYSSLVKPDQNIQFWLEQFPQAW | Q8UM99 | Porcine |
| | AETAGMGLAKQVPPQVIQLKASAAPVSVRQYP | | endogenous |
| | LSKEAREGIRPHVQRLIQQGILVPVQSPWNTPLL | | retrovirus |
| | PVRKPGTNDYRPVQDLREVNKRVQDIHPTVPNP | | |
| | YNLLCALPPQRSWYTVLDLKDAFFCLRLHPTSQ | | |
| | PLFAFEWRDPGAGRTGQLTWTRLPQGFKNSPTI | | |
| | FDEALHRDLANFRIQHPQVTLLQYVDDLLLAGA | | |
| | TKQDCLEGTKALLLELSDLGYRASAKKAQICRR | | |
| | EVTYLGYSLRGGQRWLTEARKRTVVQIPAPTTA | | |
| | KQVREFLGTAGFCRLWIPGFATLAAPLYPLTKE | | |
| | KGEFSWAPEHQKAFDAIKKALLSAPALALPDVT | | |
| | KPFTLYVDERKGVARGVLTQTLGPWRRPVAYL | | |
| | SKKLDPVASGWPVCLKAIAAVAILVKDADKLT | | |
| | LGQNITVIAPHALENIVRQPPDRWMTNARMTH | | |
| | YQSLLLTERVTFAPPAALNPATLLPEETDEPVTH | | |
| | DCHQLLIEETGVRKDLTDIPLTGEVLTWFTDGSS | | |
| | YVVKGKRMAGPPVVDGTRTIWASSLPEGTSAQ | | |
| | KAELMALTQALRLAEGKSINIYTDSRYAFATAH | | |
| | VHGAIYKQRGLLTSAGREIKNKEEILSLLEALHL | | |
| | PKRLAIIHCPGHQKAKDPISRGNQMADRVAKQA | | |
| | AQGVNLLPMIETPKAP | | |
| 8297 | TPQLDDEYRLYSPQVKPDQDIQSWLEQFPQAW | A1YTJ2 | Porcine |
| | AETAGMGLAKQVPPQVIQLKASATPVSVRQYP | | endogenous |
| | LSREAREGIWPHVQRLIQQGILVPVQSPWNTPLL | | retrovirus C |
| | PVRKPGTNDYRPVQDLREVNKRVQDIHPTVPNP | | |
| | YNLLSALPPERNWYTVLDLKDAFFCLRLHPTSQ | | |
| | PLFAFEWRDPGTGRTGQLTWTRLPQGFKNSPTI | | |
| | FDEALHRDLANFRIQHPQVTLLQYVDDLLLAGA | | |
| | TKQDCSEGTKALLLELSDLGYRASAKKAQICRR | | |
| | EVTYLGYSLRDGQRWLTEARKKTVVQIPAPTT | | |
| | AKQVREFLGTAGFCRLWIPGFATLAAPLYPLTK | | |
| | EKGEFSWAPEHQKAFDAIKKALLSAPALALPDV | | |
| | TKPFTLYVDERKGVARGVLTQTLGPWRRPVAY | | |
| | LSKKLDPIASGWPVCLKAIAAVAILVKDADKLT | | |
| | LGQNITIIAPHALENIVRQPPDRWMTNARMTQY | | |
| | QSLLLTERITFAPPAALNPATLLPEETDEPVTHD | | |
| | CHQLLIEETGVRKDLIDIPLTGEVLTWFTDGSSY | | |
| | VVEGKRMAGAAVVDGTRTIWASSLPEGTSAQK | | |
| | AELMALTQALRLADGKSINIYTDSRYAFATAHV | | |
| | HGAIYKQRGLLTSAGREIKNKEEILSLLEALHLP | | |
| | KRLAIIHCPGHQKAKDPISRGNQMADRVAKQA | | |
| | AQGVNLLPIIETPKAP | | |
| 8298 | TLQLDDEYRLYSPQVKPDQDIQSWLEQFPQAW | Q8UM96 | Porcine |
| | AETAGMGLAKQVPPQVIQLKASATPVSVRQYP | | endogenous |
| | LSREAREGIWPHVQRLIQQGILVPVQSPWNTPLL | | retrovirus |
| | PVRKPGTNDYRPVQDLREVNKRVQDIHPTVPNP | | |
| | YNLLSALPPERNWYTVLDLKDAFFCLRLHPTSQ | | |
| | PLFAFEWRDPGTGRTGQLTWTRLPQGFKNSPTI | | |
| | FDEALHRDLANFRIQHPQVTLLQYVDDLLLAGA | | |
| | TKQDCLEGTKALLLELSDLGYRASAKKAQICRR | | |
| | EVTYLGYSLRGGQRWLTEARKKTVVQIPAPTT | | |
| | AKQVREFLGTAGFCRLWIPGFATLAAPLYPLTK | | |
| | EKGEFSWAPEHQKAFDAIKKALLSAPALALPDV | | |
| | TKPFTLYVDERKGVARGVLTQTLGPWRRPVAY | | |
| | LSKKLDPVASGWPICLKAIAAVAILVKDADKLT | | |
| | LGQNITVIAPHALENIVRQPPDRWMTNARMTH | | |
| | YQSLLLTERVTFAPPAALNPATLLPEETDEPVTH | | |
| | DCHQLLIEETGVRKDLTDIPLTGEVLTWFTDGSS | | |
| | YVVEGKRMAGAAVVDGTRTIXASSLPEGTSAQ | | |
| | KAELMALTQALRLAEGKSINIYTDSRYAFATAH | | |
| | VHGAIYKQRGLLTSAGREIKNKDEILSLLEALHL | | |
| | PKRLAIIHCPGHQKAKDLISRGNQMADRIAKQA | | |
| | AQAVNLLPIIETPKAP | | |
| 8299 | TLQLDDEYRLYSPQVKPDQDIQSWLEQFPQAW | ACD35951.1 | Porcine |
| | AETAGMGLAKQVPPQVIQLKASATPVSVRQYP | | endogenous |
| | LSREAREGIWPHVQRLIQQGILVPVQSPWNTPLL | | retrovirus |
| | PVRKPGTNDYRPVQDLREVNKRVQDIHPTVPNP | | |
| | YNLLSALPPERNWYTVLDLKDAFFCLRLHPTSQ | | |
| | PLFAFEWRDPGTGRTGQLTWTRLPQGFKNSPNI | | |
| | FDEALHRDLANFRIQHPQVTLLQYVDDLLLAGA | | |
| | TKQDCLEGTKALLLELSDLGYRASAKKAQICRR | | |
| | EVTYLGYSLRGGQRWLTEARKKTVVQIPAPTT | | |
| | AKQVREFLGTAGFCRLWIPGFATLAAPLYPLTK | | |
| | EKGEFSWAPEHQKAFDAIKKALLSAPALALPDV | | |
| | TKPFTLYVDERKGVARGVLTQTLGPWRRPVAY | | |
| | LSKKLDPVASGWPVCLKAIAAVAILVKDADKL | | |
| | TLGQNITVIAPHALENIVRQPPDRWMTNARMTH | | |
| | YQSLLLTERVTFAPPAALNPATLLPEETDEPVTH | | |
| | DCHQLLIEETGVRKDLTDIPLTGEVLTWFTDGSS | | |
| | YVVEGKRMAGAAVVDGTHTIWASSLPEGTSAQ | | |
| | KAELMALTQALRLAEGKSINIYTDSRYAFATAH | | |
| | VHGAIYKQRGLLTSAGREIKNKEEILSLLEALHL | | |
| | PKRLAIIHCPGHQKAKDLISRGNQMADRVAKQ | | |
| | AAQAVNLLPIIETPKAP | | |
| 8300 | VLSTEEEYRLHEEQPKGAAPLDWVTAFPNVWA | O89815 | Mus dunni |
| | EQAGMGLAKQVPPVVVELKADATPISVRQYPM | | endogenous |
| | SKEAKEGIRPHIRRLLDQGILVACQSPWNTPLLP | | virus |
| | VRKPGTNDYRPVQDLREVNKRVLDIHPTVPNPY | | |
| | NLLSSLPPERTWYTVLDLKDAFFCLRLHPKSQL | | |
| | LFAFEWRDPEGGQTGQLTWTRLPQGFKNSPTLF | | |
| | DEALHRDLAPFRAQNPQLTLLQYVDDLLIAAAS | | |
| | KELCQQGTERLLTELGNLGYRVSAKKAQICQTE | | |
| | VIYLGYTLRGGKRWLTEARKKTVMMIPPPTTPR | | |
| | QVREFLGTAGFCRLWIPGFATLAAPLYPLTREGI | | |
| | PFEWKEEHQRAFEAIKSSLMTAPALALPDLTKS | | |
| | FVLYVDERAGIARGVLTQALGPWKRPVAYLSK | | |
| | KLDPVASGWPTCLKAIAAVALLIKDADKLTMG | | |
| | QQVTVVAPHALESIVRQPPDRWMTNARMTHY | | |
| | QSLLLNDRVTFAPPAILNPATLLPLTNDSVPVHR | | |
| | CADILAEEIGTRKDLTDQPWPGAPSWYTDGSSF | | |
| | LIEGKRRAGAAVVDGKKVIWASALPEGTSAQK | | |
| | AELIALTQALREAEGKIINIYTDSRYAFATAHIH | | |
| | GAIYRQRGLLTSAGKDIKNKEEILALLEAIHAPK | | |
| | KVAIIHCPGHQKGEDLVAKGNRMADSVAKQVA | | |
| | QGAMILTEKGNPSKS | | |
| 8301 | VLNLEEEYRLHEKPVPPSIDPSWLQLFPMVWAE | Q9TTC1 | Koala retrovirus |
| | KAGMGLANQVPPVVVELKSDASPVAVRQYPM | | |
| | SKEAREGIRPHIQRFLDLGILVPCQSPWNTPLLP | | |
| | VKKPGTNDYRPVQDLREVNKRVQDIHPTVPNP | | |
| | YNLLSSLPPSHTWYSVLDLKDAFFCLKLHPNSQ | | |
| | PLFAFEWRDPEKGNTGQLTWTRLPQGFKNSPTL | | |
| | FDEALHRDLASFRALNPQVVMLQYVDDLLVAA | | |
| | PTYRDCKEGTRRLLQELSKLGYRVSAKKAQLC | | |
| | REEVTYLGYLLKGGKRWLTPARKATVMKIPTP | | |
| | TTPRQVREFLGTAGFCRLWIPGFASLAAPLYPLT | | |
| | REKVPFTWTEAHQEAFGRIKEALLSAPALALPD | | |
| | LTKPFALYVDEKEGVARGVLTQTLGPWRRPVA | | |
| | YLSKKLDPVASGWPTCLKAIAAVALLLKDADK | | |
| | LTLGQNVLVIAPHNLESIVRQPPDRWMTNARM | | |
| | THYQSLLLNERVSFAPPAILNPATLLPVESDDTPI | | |
| | HICSEILAEETGTRPDLRDQPLPGVPAWYTDGSS | | |
| | FIMDGRRQAGAAIVDNKRTVWASNLPEGTSAQ | | |
| | KAELIALTQALRLAEGKSINIYTDSRYAFATAHV | | |
| | HGAIYKQRGLLTSAGKDIKNKEEILALLEAIHLP | | |
| | KRVAIIHCPGHQRGTDPVATGNRKADEAAKQA | | |
| | AQSTRILTETTKNQEHF | | |
| 8302 | VLNLEEEYRLHEKPVPSFVDPSWLQLFPTVWAE | ALV83309.1 | Gibbon ape |
| | RTGMGLANQVPPVVVELKSGASPVAVRQYPMS | | leukemia virus |
| | KEAREGIRPHIQKFLDLGVLVPCQSPWNTPLLPV | | |
| | KKPGTNDYRPVQDLREINKRVQDIHPTVPNPYN | | |
| | LLSSLPPSHIWYSVLDLKDAFFCLRLHPNSQPLF | | |
| | AFEWRDPEKGNTGQLTWTRLPQGFKNSPTLFD | | |
| | EALHRDLAPFRALNPQVVLLQYVDDLLVAAPT | | |
| | YEDCKEGTQKLLQELSKLGYRVSAKKAQLCQK | | |
| | EVTYLGYLLKEGKRWLTPARKATVMKIPAPTTP | | |
| | RQVREFLGTAGFCRLWIPGFASLAAPLYPLTKES | | |
| | FPFVWTEEHQKAFDHIKEALLSAPALALPDLTK | | |
| | PFTLYVDERAGMARGVLTQTLGPWRRPVAYLS | | |
| | KKLDPVASGWPTCLKAVAAVALLLKDADKLTL | | |
| | GQKVTVIASHSLESIVRQPPDRWMTNARMTHY | | |
| | QSLLLNERVSFAPPAVLNPATLLPVESEATPVHR | | |
| | CSEILAEETGTRRDLKDQPLPGVSAWYTDGSSFI | | |
| | VEGKRRAGAAIVDGKRTVWASSLPEGTSAQKA | | |
| | ELVALTQALRLAKGRNINIYTDSRYAFATAHIH | | |
| | GAIYKQRGLLTSAGKDIKNKEEILALLEAIHLPK | | |
| | QVAIIHCPGHQRGNNPVATGNRRADEAAKQAA | | |
| | LSTRVLAEITKLQEP | | |
| 8303 | VLNLEEEYRLHEKPVPSFVDPSWLQLFPTVWAE | ALV83306.1 | Gibbon ape |
| | RAGMGLANQVPPVVVELKSGASPVAVRQYPMS | | leukemia virus |
| | KEAREGIRPHIQKFLDLGVLVPCQSPWNTPLLPV | | |
| | KKPGTNDYRPVQDLREINKRVQDIHPTVPNPYN | | |
| | LLSSLPPSHIWYSVLDLKDAFFCLRLHPNSQPLF | | |
| | AFEWRDPEKGNTGQLTWTRLPQGFKNSPTLFD | | |
| | EALHRDLAPFRVLNPQVVLLQYVDDLLVAAPT | | |
| | YEDCKEGTQKLLQELSKLGYRVSAKKAQLCQK | | |
| | EVTYLGYLLKEGKRWLTPARKATVMKIPAPTTP | | |
| | RQVREFLGTAGFCRLWIPGFASLAAPLYPLTKES | | |
| | FPFVWTEEHQKAFDHIKEALLSAPALALPDLTK | | |
| | PFTLYVDERAGMARGVLTQTLGPWRRPVAYLS | | |
| | KKLDPVASGWPTCLKAVAAVALLLKDADKLTL | | |
| | GQKVTVIASHSLESIVRQPPDRWMTNARMTHY | | |
| | QSLLLNERVSFAPPAVLNPATLLPVESEATPVHR | | |
| | CSEILAEETGTRRDLKDQPLPGVSAWYTDGSSFI | | |
| | AEGKRRAGAAIVDGKRTVWASSLPEGTSAQKA | | |
| | ELVALTQALRLAKGRNINIYTDSRYAFATAHIH | | |
| | GAIYKQRGLLTSAGKDIKNKEEILALLEAIHLPK | | |
| | QVAIIHCPGHQRGSNPVATGNRRADEAAKQAA | | |
| | LSTRVLAETTKPQEP | | |
| 8304 | VLNLEEEYRLHEKPVPSSIDPSWLQLFPTVWAE | P21414 | Gibbon ape |
| | RAGMGLANQVPPVVVELRSGASPVAVRQYPMS | | leukemia virus |
| | KEAREGIRPHIQKFLDLGVLVPCRSPWNTPLLPV | | |
| | KKPGTNDYRPVQDLREINKRVQDIHPTVPNPYN | | |
| | LLSSLPPSYTWYSVLDLKDAFFCLRLHPNSQPLF | | |
| | AFEWKDPEKGNTGQLTWTRLPQGFKNSPTLFD | | |
| | EALHRDLAPFRALNPQVVLLQYVDDLLVAAPT | | |
| | YEDCKKGTQKLLQELSKLGYRVSAKKAQLCQR | | |
| | EVTYLGYLLKEGKRWLTPARKATVMKIPVPTTP | | |
| | RQVREFLGTAGFCRLWIPGFASLAAPLYPLTKES | | |
| | IPFIWTEEHQQAFDHIKKALLSAPALALPDLTKP | | |
| | FTLYIDERAGVARGVLTQTLGPWRRPVAYLSK | | |
| | KLDPVASGWPTCLKAVAAVALLLKDADKLTLG | | |
| | QNVTVIASHSLESIVRQPPDRWMTNARMTHYQ | | |
| | SLLLNERVSFAPPAVLNPATLLPVESEATPVHRC | | |
| | SEILAEETGTRRDLEDQPLPGVPTWYTDGSSFIT | | |
| | EGKRRAGAPIVDGKRTVWASSLPEGTSAQKAE | | |
| | LVALTQALRLAEGKNINIYTDSRYAFATAHIHG | | |
| | AIYKQRGLLTSAGKDIKNKEEILALLEAIHLPRR | | |
| | VAIIHCPGHQRGSNPVATGNRRADEAAKQAALS | | |
| | TRVLAGTTKPQEPI | | |
| 8305 | VLSLEEEYRLHEKPVPTSIDPSWLQLFPTVWAER | O70652 | Gibbon ape |
| | AGMGLANRVPPVVVELKSGASPVAVRQYPMSK | | leukemia virus |
| | EAREGIRPHIQRFLDLGVLVPCRSPWNTPLLPVK | | |
| | KPGTNDYRPVQDLREINKRVQDIHPTVPNPYNL | | |
| | LSSLPPSHTWYSVLDLKDAFFCLKLHPNSQSLFA | | |
| | FEWKDPEKGNTGQLTWTRLPQGFKNSPTLFDE | | |
| | ALHRDLALFRAHNPQVKLLQYVDDLLVAAPTY | | |
| | QDCKEGTQKLLQELSKLGYRVSAKKAQLCQKE | | |
| | VTYLGYLLKEGKRWLTPARKATVMKIPAPTTP | | |
| | RQVREFLGTAGFCRLWIPGFASMAAPLYPLTKE | | |
| | SIPFIWTEEHQKAFDLIKKALLSAPALALPDLTK | | |
| | PFTLYVDERAGVARGVLTQTLGPWRRPVAYLS | | |
| | KKLDPVASGWPTCLKAVAAVALLLKDADKLTL | | |
| | GQNVTVIASHSLESIVRQPPDRWMTNARMTHY | | |
| | QSLLLNERVSFAPPAVLNPATLLPVESEATPVHR | | |
| | CSEILAEETGTRQDLKDQPLPGVPTWYTDGSSFI | | |
| | AEGKRKAGAAIVDGKRTVWASSLPEGTSAQKA | | |
| | ELVALTQALRLAEGRNINIYTDSRYAFATAHIH | | |
| | GAIYKQRGLLTSAGKDIKNKEEILALLEAIHLPK | | |
| | RVAIIHCPGHQKGNDPVATGNRRADEAAKQAA | | |
| | LSTRVLAETTKPQEP | | |
| 8306 | VLGLEEEYRLHEKPVPSSVDPSWLQLFPDVWAE | QDA02050.1 | Flying-fox |
| | KGGMGLANRVPPIVVELKSDALPVAVRQYPMS | | retrovirus |
| | REAREGIRPHIQRFLDLGVLVPCQSPWNTPLLPV | | |
| | KKPGTSDYRPVQDLREINKRVQDIHPTVPNPYN | | |
| | LLSSLPPNHTWYSVLDLKDAFFCLKLHPNSQLL | | |
| | FAFEWRDPEKGHTGQLTWTRLPQGFKNSPTLFD | | |
| | EALHRDLASFRASNPQVVLLQYVDDLLVAAPT | | |
| | YKDCKEGTQKLLQELSELGYRVSAKKAQLCQR | | |
| | EVTYLGYLLKEGKRWLTPARKATVMEIPTPTTP | | |
| | RQVREFLGTAGFCRLWIPGFASLAAPLYPLTKES | | |
| | TPFLWTEEHRRAFDQIKEALLTAPALALPDLTKP | | |
| | FALYVDERAGVARGVLTQTLGPWRRPVAYLSK | | |
| | KLDPVASGWPTCLKAVAAVALLLKDADKLTLG | | |
| | QSVTVIASHSLESIVRQPPDRWMTNARMTHYQS | | |
| | LLLNERVSFAPPAVLNPATLLPAESGAAPVHECS | | |
| | EILAEETGTRQDLTDQPLPGVPAWYTDGSSFITE | | |
| | GKRRAGAAIVDGKRTVWMSSLPEGTSAQKAEL | | |
| | IALTQALRLADGKDINIYTDSRYAFATAHIHGAI | | |
| | YRQRGLLTSAGKEIKNKEEILALLEAIHLPKRVA | | |
| | IIHCPGHQKGNDPVAIGNRRADEAAKQAALAV | | |
| | RVLAETIEPQGQ | | |
| 8307 | VLNLEEEYRLHEKPAPPSIDPFWLQLFPNVWAE | QJT93247.1 | Hervey pteropid |
| | QGGMGLANQVPPVVVELKSDASPVAVRQYPM | | gammaretrovirus |
| | SKEAREGIRPHIQRFLDLGVLVPCQSPWNTPLLP | | |
| | VKKPGTNDYRPVQDLREINKRVQDIHPTVPNPY | | |
| | NLLSSLPPSHTWYSVLDLKDAFFCLKLHPNSQL | | |
| | LFAFEWRDPEKGHTGQLTWTRLPQGFKNSPTLF | | |
| | DEALHRDLASFRASNPQVILLQYVDDLLVAAPT | | |
| | YEDCKEGTQKLLQELSELGYRVSAKKAQLCQK | | |
| | EVTYLGYLLKEGKRWLTPARKATVMRIPTPITP | | |
| | RQVREFLGTAGFCRLWIPGFASLAAPLYPLTKES | | |
| | VPFLWTEEHQRAFDHIKEALLTAPALALPDLTK | | |
| | PFALYVDEKAGVARGVLTQTLGPWRRPVAYLS | | |
| | KKLDPVASGWPTCLKAVAAVALLLKDADKLTL | | |
| | GQNVTVIASHSLESIVRQPPDRWMTNARMTHY | | |
| | QSLLLNERVSFAPPAVLNPATLLPAESEAALVH | | |
| | DCSEILAEETGTRQDLTDQPLPGVPAWYTDGSS | | |
| | FIAEGKRRAGAAIVDNKRTVWMSSLPEGTSAQ | | |
| | KAELIALTQALRLADGKDINIYTDSRYAFATAHI | | |
| | HGAIYRQRGLLTSAGKEIKNKEEILALLEAIHLP | | |
| | RRVAIVHCPGHQKGNDPIALGNRRADEAAKQA | | |
| | ALSVRVLAETTGPQGP | | |
| 8308 | VLNLEEEYRLHEKPVPSSIDPLWLQLFPNVWAE | QJT93250.1 | Macroglossus |
| | KGGMGLASQVPPVVVELKSDASPVAVRQYPMS | | minimus |
| | REAQEGIRPHIQRFLDLGVLVPCQSPWNTPLLPV | | gammaretrovirus |
| | KKPGTNDYRPVQDLREVNKRVQDIHPTVPNPY | | |
| | NLLSSLPPSHTWYTVLDLKDAFFCLKLHPNSQP | | |
| | LFAFEWRDPEKGHTGQLTWTRLPQGFKNSPTLF | | |
| | DEALHRDLASFRASNPQVVLLQYVDDLLVAAP | | |
| | TYEDCKEGTQKLLQELSNLGYRVSAKKAQLCQ | | |
| | KEVTYLGYLLKEGQRWLTPARKATVMGIPTPT | | |
| | TPRQVREFLGTAGFCRLWIPGFASLAAPLYPLTK | | |
| | ESTPFLWTEEHQKAFDCIKEALLTAPALALPDLT | | |
| | KPFALYVDERDGVARGVLTQTLGPWRRPVAYL | | |
| | SKKLDPVASGWPTCLKAVAAVALLLKDADKLT | | |
| | LGQNVTVIASHSLESIVRQPPDRWMTNARMTH | | |
| | YQSLLLNERVSFAPPAVLNPATLLPAESEAAPV | | |
| | HTCSEILAEETGTRKDLTDQPLPGVPAWYTDGS | | |
| | SFITEGKRRAGAAIVDSKRTVWMSSLPEGTSAQ | | |
| | KAELIALTQALRLANGRDINIYTDSRYAFATAHI | | |
| | HGAIYRQRGLLTSAGKEIKNREEILALLEAIHLP | | |
| | RRVAIIHCPGHQKGNDPVAVGNRRADEAAKQA | | |
| | ALSVQVLAEITKPQEL | | |
| 8309 | TLPLAEEYLLYEGPHDTGDRWLEKWKDELPGV | AGV92853.1 | Galidia ERV |
| | WAETNPPGLAKDRPPIHVQLMSTAQPIRVRQYP | | |
| | MTLEARRGVRENIRKLRAAGILVPCHSPWNTPL | | |
| | LPVRKAETGQYRMVQDLREVNKRVETIHPTVP | | |
| | NPYTLLSLLPPDHIWYSVLDLKDAFFCLPLAPGS | | |
| | QPLFAFEWSDPEEGESGQLTWTRLPQGFKNSPT | | |
| | LFDEALSHDLQSYRTGHPEVTLLQYMDDLIVAA | | |
| | RSEAECAQATRDLLETLGDQGYRVSAKKAQLC | | |
| | SQQVTYLGFRLKGGTRTLTESRIKAIVQIPSPKT | | |
| | KRQVREFLGTVGYCRLWIPGFAELAKPLHAVA | | |
| | GGGARPLTWTKTEEEAFQALKSALLQAPALSLP | | |
| | DLEKPFQLFVAENKGVAKGVLTQRIGPWKRPV | | |
| | AYLSRKLDPVAAGWPGCLRAIAAAALLVKEAS | | |
| | KLTFGQNLEVTSAHNLESLLRSPPDRWMTNTRV | | |
| | TQYQVLLLDPPRVSFRQTAALNPATLLPEADES | | |
| | LPFHQCEDTLDALTTLRPDLTDRPLLDAEVTLFT | | |
| | DGSSFVDQGXRHAGAAIVTLDSTIWAEALPKGT | | |
| | SAQRAELIALTKALLWGEDKRVNIYTDSRYAFA | | |
| | TLHVHGALYKERGLLTTGGKEIKNAPEILALLSS | | |
| | VWKPKKVAVIHCRGHQKNDTNIARGNQRADR | | |
| | VAKEVARGEIAPVLTLQEPNPV | | |
| 8310 | SSPLVEEYRLFVEQPAQNLALLDLWREDIPEVW | AGV92856.1 | Echidna ERV |
| | AESNPPGLATTQVPVHVQLTSTALPIRIRQYPISL | | Duck infectious |
| | EARRSLRGSIRKFKAAGILKPVHSPWNTPLLPVR | | |
| | KTGTSEYRMVQDLREVNKRVETIHPTVPNPYTL | | |
| | LSLLPPDRTWYSVLDLKDAFFCIPLTCQSQLLFA | | |
| | FEWIDIEEGESGQLTWTRLPQGFKNSPTLFDEAL | | |
| | SRDLQGYRFDHPTVTLLQYVDDLLIAARSRDEC | | |
| | LQATRDLLVTLGSMGYRVSGSKAQLCQEEVTY | | |
| | LGFRIKDGTRTLAQSRVQAILQIPAPKTKKQVRE | | |
| | FLGTVGYCRLWIPSFAELAQPLYAATRGADAPL | | |
| | RWTGTEEEAFQRLKTALLQPPALALPNLDKPFQ | | |
| | LFVDEAKGVAKGVLMQTLGPWKRPVAYLSRK | | |
| | LDPLAAGWPRCLRAIAAAALLSKEASKLTFEQS | | |
| | LEITSSHNLEGLLRTPPDKWLTNARVTQYQVLL | | |
| | LDPPRVIFKQTAALNPATLLPATDDSLPLHHCA | | |
| | DTLDALTTTRPDLTDQPLADAEATLFTDGSSYV | | |
| | KKAEYAGAAVVTTNSIVWAEALPRGTSAQRAE | | |
| | LIALTKALEWSRDKTVNIYTDSRYAFATLHVHA | | |
| | MIYKERGLLTAGGKAIKNASEILALLTAIWLPKR | | |
| | VAVIHCRGHQQGESLEALGNRLADKTAREVAK | | |
| | KSPAIQASLCDPPRTP | | |
| 8311 | TAPLEEEYRLFLEAPIQNVTLLEQWKREIPKVW | AGV92859.1 | anemia virus] |
| | AEINPPGLASTQAPIHVQLLSTALPVRVRQYPITL | | |
| | EARRSLRETIRKFRAAGILRPVHSPWNTPLLPVR | | |
| | KPGTSEYRMVQDLREVNKRVETIHPTVPNPYTL | | |
| | LSLLPPDRTWYSVLDLKDAFFCIPLAPESQLIFAF | | |
| | EWTDAEEGESGQLTWTRLPQGFKNSPTLFDEAL | | |
| | NRDLQGFRLDHPSVSLLQYVDDLLIAADTQAAC | | |
| | LSATRDLLMTLAELGYRVSGKKAQLCQEEVTY | | |
| | LGFKIHKGSRTLSNSRTQAILQIPVPKTKRQVRE | | |
| | FLGTIGYCRLWIPGFAELAQPLYAATRGGNDPL | | |
| | EWGEKEEEAFQSLKLALTQPPALALPSLDKPFQ | | |
| | LFIEETGGAAKGVLTQTLGPWKRPVAYLSKRLD | | |
| | PVAAGWPRCLRAIAAAALLTREASKLTFGQDIEI | | |
| | TSSHNLESLLRSPPDRWLTNARITQYQVLLLDPP | | |
| | RVRFKQTAALNPATLLPETDDTLPIHHCLDTLDS | | |
| | LTSTRPDLTDQPLAQAEATLFTDGSSYIRDGKR | | |
| | YAGAAVVTLDSVVWAEPLPIGTSAQKAELIALT | | |
| | KALEWSKDKSVNIYTDSRYAFATLHVHGMIYK | | |
| | ERGLLTAGGKAIXNAPEILALLTAVWLPKRVAV | | |
| | MHCRGHQKDDAPTSAGNRRADEVAREVAIRPL | | |
| | SVQATVSDAPDMP | | |
| 8312 | TAPLEEEYRLFLEAPIQNVTLLEQWKREIPKVW | AHC55379.1 | Reticulo |
| | AEINPPGLASTQAPIHVQLLSTALPVRVRQYPITL | | endotheliosis |
| | EARRSLRETIRKFRAAGILRPVHSPWNTPLLPVR | | virus |
| | KPGTSEYRMVQDLREVNKRVETIHPTVPNPYTL | | |
| | LSLLPPDRTWYSVLDLKDAFFCIPLAPKSQLIFA | | |
| | FEWTDAEEGESGQLTWTRLPQGFKNSPTLFDEA | | |
| | LNRDLQGFRLEHPSVSLLQYVDDLLIAADTQAA | | |
| | CLSATRDLLMTLAELGYRVSGKKAQLCQEEVT | | |
| | YLGFKIHKGSRTLSNSRTQAILQIPVPKTKRQVR | | |
| | EFLGTIGYCRLWIPGFAELAQPLYAATRGGNDP | | |
| | LEWGEKEEEAFQSLKLALTQPPALALPSLDKPF | | |
| | QLFIEETGGAAKGVLTQALGPWKRPVAYLSKR | | |
| | LDPVAAGWPRCLRAIAAAALLTREASKLTFGQ | | |
| | DIEITSSHNLESLLRSPPDRWLTNARITQYQVLLL | | |
| | DPPRVRFKQTAALNPATLLPETDDTLPIHHCLDT | | |
| | LDSLTSTRPDLTDQPLAQAEATLFTDGSSYIRDG | | |
| | KRYAGAAVVTLDSVIWAEPLPIGTSAQKAELIA | | |
| | LTKALEWSKDKSVNIYTDSRYAFATLHVHGMI | | |
| | YKERGLLTAGGKAIKNAPEILALLTAVWLPKRV | | |
| | AVMHCRGHQKDDAPTSAGNRRADEVAREVAI | | |
| | RPLSIQATVSDAPDMP | | |
| 8313 | TAPLEEEYRLFLEAPIQNVTLLEQWKREIPKVW | ASH96780.1 | Reticulo |
| | AEINPPGLASTQAPIHVQLLSTALPVRVRQYPITL | | endotheliosis |
| | EARRSLRETIRKFRAAGILRPVHSPWNTPLLPVR | | virus |
| | KPGTSEYRMVQDLREVNKRVETIHPTVPNPYTL | | |
| | LSLLPPDRTWYSVLDLKDAFFCIPLAPKSPLIFAF | | |
| | EWTDAEEGESGQLTWTRLPQGFKNSPTLFDEAL | | |
| | NRDLQGFRLDHPSVSLLQYVDDLLIAADTQAAC | | |
| | LSATRDLLMTLAELGYRVSGKKAQLCQEEVTY | | |
| | LGFKIHKGSRTLSNSRIQAILQIPVPKTKRQVREF | | |
| | LGTIGYCRLWIPGFAELAQPLYAATRGGNDPLE | | |
| | WGEKEEEAFQSLKLALTQPPALALPSLDKPFQL | | |
| | FIEETGGAAKGVLTQALGPWKRPVAYLSKRLDP | | |
| | VAAGWPRCLRAIAAAALLTREASKLTFGQDIEI | | |
| | TSSHNLESLLRSPPDRWLTNARITQYQVLLLDPP | | |
| | RVRFKQTAALNPATLLPETDDTLPIHHCLDTLDS | | |
| | LTSTRPDLTDQPLAQAEATLFTDGSSYIPHGKRY | | |
| | AGAAVVTLDSVIWAEPLPIGTSAQKAELIALTK | | |
| | ALEWSKDKSVNIYTDSRYAFATLHVHGMIYKE | | |
| | RGLLTAEGKAIKNAPEILALLTAVWLPKRVAV | | |
| | MHCRGHQKDDAPTSAGNRRADEVAREVAIRPL | | |
| | SIQATVFDAPDMP | | |
| 8314 | TAPLEEEYRLFLEAPIQNVTLLEQWKREIPKVW | P03360 | Reticulo |
| | AEINPPGLASTQAPIHVQLLSTALPVRVRQYPITL | | endotheliosis |
| | EAKRSLRETIRKFRAAGILRPVHSPWNTPLLPVR | | virus |
| | KSGTSEYRMVQDLREVNKRVETIHPTVPNPYTL | | |
| | LSLLPPDRIWYSVLDLKDAFFCIPLAPESQLIFAF | | |
| | EWADAEEGESGQLTWTRLPQGFKNSPTLFDEA | | |
| | LNRDLQGFRLDHPSVSLLQYVDDLLIAADTQAA | | |
| | CLSATRDLLMTLAELGYRVSGKKAQLCQEEVT | | |
| | YLGFKIHKGSRSLSNSRTQAILQIPVPKTKRQVR | | |
| | EFLGTIGYCRLWIPGFAELAQPLYAATRGGNDP | | |
| | LVWGEKEEEAFQSLKLALTQPPALALPSLDKPF | | |
| | QLFVEETSGAAKGVLTQALGPWKRPVAYLSKR | | |
| | LDPVAAGWPRCLRAIAAAALLTREASKLTFGQ | | |
| | DIEITSSHNLESLLRSPPDKWLTNARITQYQVLL | | |
| | LDPPRVRFKQTAALNPATLLPETDDTLPIHHCLD | | |
| | TLDSLTSTRPDLTDQPLAQAEATLFTDGSSYIRD | | |
| | GKRYAGAAVVTLDSVIWAEPLPIGTSAQKAELI | | |
| | ALTKALEWSKDKSVNIYTDSRYAFATLHVHGM | | |
| | IYRERGLLTAGGKAIKNAPEILALLTAVWLPKR | | |
| | VAVMHCKGHQKDDAPTSTGNRRADEVAREVA | | |
| | IRPLSTQATISDAPDMP | | |
| 8315 | TAPLEEEYRLFLEAPIQNVTLLEQWKREIPKVW | ACJ65653.1 | Reticulo |
| | AEINPPGLASTQAPIHVQLLSTALPVRVRQYPITL | | endotheliosis |
| | EAKRSLRETIRKFRAAGILRPVHSPWNTPLLPVR | | virus] |
| | KSGTSEYRMVQDLREVNKRVETIHPTVPNPYTL | | |
| | LSLLPPDRIWYSVLDLKDAFFCIPLAPESQLIFAF | | |
| | EWADAEEGESGQLTWTRLPQGFKNSPTLFDEA | | |
| | LNRDLQGFRLDHPFVSLLQYVDDLLIAADTQAA | | |
| | CLSATRDLLMTLAELGYRVSGKKAQLCQEEVT | | |
| | YLGFKIHKGSRTLSNSRTQAILQIPVPKTKRQVR | | |
| | EFLGTIGYCRLWIPGFAELAQPLYAATRGGNDP | | |
| | LVWGEKEEGAFQSLKLALTQPPALALPSLDKPF | | |
| | QLFVEETGGAAKGVLTQALGPWKRPVAYLSKR | | |
| | LDPVAAGWPRCLRAIAAAALLTREASKLTFGQ | | |
| | DIEITSSHNLESLLRSPPDRWLTNARITQYQVLLL | | |
| | DPPRVRFKQTAALNPATLLPETDDTLPIHHCLDT | | |
| | LDSLTSTRPDLTDQPLAQAEATLFTDGSSYIRDG | | |
| | KRYTGAAVVTLDSVIWAEPLPIGTSAQKAELIA | | |
| | LTKALEWSKDKSVNIYTDSRYAFATLHVHGMI | | |
| | YRERGLLTAGGKAIKNAPEILALLTAVWLPKRV | | |
| | AVMHCKGHQKDDAPTSTGNRRADEVAREVAIR | | |
| | PLSTQATISDAPDMP | | |
| 8316 | TAPLEEEYRLFLEAPIQNVTLLEQWKREIPKVW | ACT75574.1 | Reticulo |
| | AEINPPGLASTQAPIHVQLLSTALPVRVRQYPITL | | endotheliosis |
| | EAKRSLRETIRKFRAAGILRPVHSPWNTPLLPVR | | virus] |
| | KSGTSEYRMVQDLREVNKRVETIHPTVPNPYTL | | |
| | LSLLPPDRIWYSVLDLKDAFFCIPLAPESQLIFAF | | |
| | EWADAEEGESGQLTWTRLPQGFKNSPTLFDEA | | |
| | LNRDLQGFRLDHPFVSLLQYVDDLLIAADTQAA | | |
| | CLSATRDLLMTLAELGYRVSGKKAQLCQEEVT | | |
| | YLGFKIHKGSRTLSNSRTQAILQIPVPKTKRQVR | | |
| | EFLGTIGYCRLWIPGFAELAQPLYAATRGGNDP | | |
| | LVWGEKEEESFQSLKLALTQPPALALPSLDKPF | | |
| | QLFVEETGGAARGVLTQALGPWKRPVAYLSKR | | |
| | LDPVAAGWPRCLRAIAAAALLTREASKLTFGQ | | |
| | DIEITSSHNLESLLRSPPDRWLTNARITQYQVLLL | | |
| | DPPRVRFKQTAALNPATLLPETDDTLPIHHCLDT | | |
| | LDSLTSTRPDLTDQPLAQAEATLFTDGSSYIRDG | | |
| | KRYTGAAVVTLDSVIWAGPLPIGTSAQKAELIA | | |
| | LTKALEWSKDKSVNIYTDSRYAFATLHVHGMI | | |
| | YRERGLLTAGGKAIKNAPEILALLTAVWLPKRV | | |
| | AVMHCKGHQKGDAPTSTGNRRADEVAREVAIR | | |
| | PLSTQATISDAPDMP | | |
| 8317 | TAPLEEEYRLFLEAPIQNVTLLEQWKREIPKVW | AUS82407.1 | Reticulo |
| | AEIIPPGLASTQAPIHVQLLSTALPVRVRQYPITL | | endotheliosis |
| | EAKRSLRETIRKFRAAGILRPVHSPWNTPLLPVR | | virus] |
| | KSGTSEYRMVQDLREVNKRVETIHPTVPNPYTL | | |
| | LSLLPPDRIWYSVLDLKDAFFCIPLAPESQLIFAF | | |
| | EWADAEEGESGQLTWTRLPQGFKNSPTLFDEA | | |
| | LNRDLQGFRLDHPFVSLLQYVDDLLIAADTQAA | | |
| | CLSATRDLLMTLAELGYRVSGKKAQLCQEEVT | | |
| | YLGFKIHKGSRTLSNSRTQAILQIPVPKTKRQVR | | |
| | EFLGTIGYCRLWIPGFAELAQPLYAATRGGNDP | | |
| | LVWGEKEEEALQSLKLALTQPPALALPSLDKPF | | |
| | QLFVEETGGAAKGVLTQALGPWKRPVAYLSKR | | |
| | LDPVAAGWPRCLRAIAAAALLTREASKLTFGQ | | |
| | DIEITSSHNLESLLRSPPDRWLTNARITQYQVLLL | | |
| | DPPRVRFKQTAALNPATLLPETDDTLPIHHCLDT | | |
| | LDSLTSTRHDLTDQPLAQAEATLFTDGSSYIRDG | | |
| | KRYTGAAVVTLGSVIWAEPLPIGTSAQKAELIA | | |
| | LTKALEWSKDKSVNIYTDSRYAFATLHVHGMI | | |
| | YRERGLLTAGGKAIKNAPEILALLTAVWLPKRV | | |
| | AVMHCKGHQKDDAPTSTGNRRADEVAREVAIR | | |
| | PLSTQATISDAPDMP | | |
| 8318 | TTLVPLQDYQERLLKQTAFPEQHRKRLQTLFLK | AXY87475 | Simian foamy |
| | YDALWQHWENQVGHRRIKPHHIATGTVAPRPQ | | virus |
| | KQYPINPKAKPSIQIVINDLLKQGVLIQQNSTMN | | |
| | TPVYPVPKPDGKWRMVLDYREVNKTIPLIAAQ | | |
| | NQHSAGILSSIYRGKYKTTLDLSNGFWAHSITPE | | |
| | SYWLTAFTWQGKQYCWTRLPQGFLNSPALFTA | | |
| | DVVDLLKEIPNVQAYVDDIYISHDDPEEHLEQL | | |
| | EKVFSILLNAGYVVSLKKSEIAQYEVEFLGFNIT | | |
| | KEGRGLTETFKQKLLNITPPKDLKQLQSILGLLN | | |
| | FARNFIINFSELVKPLYSIISNAQGKYITWTEENS | | |
| | NQLQHIIDVLNMAENLEERNPETRLIVKVNASPS | | |
| | AGYIRFYNEHSKRPIMYINYVFTKAEIKFTPTEK | | |
| | LLTTIHKALIKALDIAMGQEILVYSPITSMTKIQK | | |
| | TPLPERKALPIRWITWMTYLEDPRIFFHYDKTLP | | |
| | ELQQIPAVTEDVVYKTKHPSEFQRVFYTDGSAI | | |
| | KHPDITKSHSAGMGIAETQFSPEFKVLNKWSIPL | | |
| | GDHTAQLAEIAAEEFACKKALKITGPVLIVTDSF | | |
| | YVAESANKELPYWQSNGFLNNKKKPLKHVSK | | |
| | WKSIAECLQLKPDITIIHEKGHQPTATSFHTEGN | | |
| | SLADKLATQGSYVVNTNTTPSLDAELDQLLQG | | |
| | QYPKGYPKHYSYKLQEGHVVVERPNGIRIIPPK | | |
| | ADRSTIILQAHNIAHTGRDSTFLKVTSKYWWPN | | |
| | LRKDVVKVIRQCKQCLVTNQAVLTAPPILRPE | | |
| 8319 | TVLVPLQDYQERLLKQTTLPKEQKDQLEKLFLK | YP_0095132 | Rhesus macaque |
| | YDALWQHWENQVGHRRIKPHNIATGTLAPRPQ | 42 | simian foamy |
| | KQYPINPKAKPSIQIVIDDLLKQGVLIQQNSTMN | | virus |
| | TPVYPVPKPDGKWRMVLDYREVNKTIPLIAAQ | | |
| | NQHSAGILSSIYRGKYKTTLDLTNGFWAHPITPE | | |
| | SYWLTAFTWQGKQYCWTRLPQGFLNSPALFTA | | |
| | DVVDLLKEVPNVQAYVDDIYMSHDDPQEHLEQ | | |
| | LEKVFSILLNAGYVVSLKKSEIAQREVEFLGFNI | | |
| | TKEGRGLTETFKQKLLNVIPPKDLKQLQSILGLL | | |
| | NFARNFIPNYSELVKPLYTIVANANGKFISWTEE | | |
| | NSNQLQYIISVLNQADNLEERNPETRLILKVNSS | | |
| | PSAGYIRYYNEGSKRPIMYVNYVFSKAEVKFTQ | | |
| | TEKMLTTMHKGLIKAMDLAMGQEILVYSPIVS | | |
| | MTKIQKTPLPERKALPVRWITWMTYLEDPRIQF | | |
| | HYDKTLPELQQTPSVTEDVIAKTKHPSEFAMVF | | |
| | YTDGSAIKHPDINKSHSAGMGIAQVQFQPEYKV | | |
| | IHQWSIPLGDHTAQLAEIAAVEFACKKALKISGP | | |
| | VLIVTDSFYVAESANKELSYWKSNGFLNNKKKP | | |
| | LKHVSKWKSIAECLQLKPDITIIHEKGHQQPMTT | | |
| | LHTEGNNLADKLATQGSYVVHCNTTPSLDAEL | | |
| | DQLLQGHNPPGYPKQYKYTLEDNKIIVERPNGQ | | |
| | RIVPPKSDREKIISMAHNIAHTGRDATFLKVSSK | | |
| | YWWPNLRKDVVKVIRQCKQCLVTNAANLTSPP | | |
| | ILRPE | | |
| 8320 | TILVPLQDYQSRILEKTALSEEFKKQLQTLFLKY | YP_0095085 | Western lowland |
| | DNLWQHWENQVCHRKIRPHNIATGDYPPRPQK | 71 | gorilla simian |
| | QYPINPKARSSIQVVIDDLLKQGVLVQQNSTMN | | foamy virus |
| | TPVYPIPKPDGRWGMVLDYREVNKTIPLIAAQN | | |
| | QHSAGILATIVRKKYKTTLVLANGFWAHPITPES | | |
| | YWLTAFIWQGKQYCWTRLPQGFLNSPALFTAD | | |
| | VVDLLKEISNVQAYVDDIYLSHDDPQEHLDQLE | | |
| | KVFQILLQAGYVVSLKKSEVAQKTVEFLGFNIT | | |
| | KEGRGLTEAFKAKLLDITPPKDLKQLQSILGLLN | | |
| | FARNFILNFAELVKPLYSLISSAKGKYIEWSNEN | | |
| | TVQLQTIIKALNNADNLEERIPEKRLIIKVNTSPS | | |
| | AGYVRYYNETGKKPIMYLNYVFSKAELKFTLL | | |
| | EKLLTTMHKALIKAMDLAMGQEILVYSPVVSM | | |
| | TKIQKTPIPERKALPIRWITWMTYLEDPRIQFHY | | |
| | DKTLPELKNIPDVLTENSSKIMIHPSQYNSVFYT | | |
| | DGSAIRSPDPTKSHNAGMGIVQVKFSPELQVINQ | | |
| | WSIPLGNHTAQMAEIAAVEFACKKALKITGPVL | | |
| | IITDSFYVAESTNKELPYWKSNGFVNNKKKPLK | | |
| | HVSKWKSIAECLSLKPDITIQHERGHQPIYTSIHT | | |
| | EGNALADKLATQGSYVVNNNDKKPNLDAELD | | |
| | HLIQGKYPKGYPKQYTYYMEDGKVKVNRPEGT | | |
| | KIIPPSLERAGIVQKAHNLAHTGREATLLKIANL | | |
| | YWWPNMRKDVVRQLGRCQQCLVTNAFNQTSG | | |
| | PILRPT | | |
| 8321 | TILVPLQEYQEKILSKTALPEDQKQQLKTLFVKY | YP_0095085 | Eastern |
| | DNLWQHWENQVGHRKIRPHNIATGDYPPRPQK | 51 | chimpanzee |
| | QYPINPKAKPSIQIVIDDLLKQGVLTPQNSTMNT | | simian foamy |
| | PVYPVPKPDGRWRMVLDYREVNKTIPLTAAQN | | virus |
| | QHSAGILATIVRQKYKTTLDLANGFWAHPITPES | | |
| | YWLTAFTWQGKQYCWTRLPQGFLNSPALFTAD | | |
| | VVDLLKEIPNVQVYVDDIYLSHDDPKEHVQQLE | | |
| | KVFQILLQAGYVVSLKKSEIGQKTVEFLGFNITK | | |
| | EGRGLTDTFKTKLLNITPPKDLKQLQSILGLLNF | | |
| | ARNFIPNFAELVQPLYNLIASAKGKYIEWSEENT | | |
| | KQLNMVIEALNTASNLEERLPEQRLVIKVNTSPS | | |
| | AGYVRYYNETGKKPIMYLNYVFSKAELKFSML | | |
| | EKLLTTMHKALIKAMDLAMGQEILVYSPIVSMT | | |
| | KIQKTPLPERKALPIRWITWMTYLEDPRIQFHYD | | |
| | KTLPELKHIPDVYTSSQSPVKHPSQYEGVFYTD | | |
| | GSAIKSPDPTKSNNAGMGIVHATYKPEYQVLNQ | | |
| | WSIPLGNHTAQMAEIAAVEFACKKALKIPGPVL | | |
| | VITDSFYVAESANKELPYWKSNGFVNNKKKPL | | |
| | KHISKWKSIAECLSMKPDITIQHEKGHQPTNTSI | | |
| | HTEGNALADKLATQGSYVVNCNTKKPNLDAEL | | |
| | DQLLQGHYIKGYPKQYTYFLEDGKVKVSRPEG | | |
| | VKIIPPQSDRQKIVLQAHNLAHTGREATLLKIAN | | |
| | LYWWPNMRKDVVKQLGRCQQCLITNASNKAS | | |
| | GPILRPD | | |
| 8322 | TIKLPVQDLKNTLVSQANIGKEDKIKLAKLLDK | YP_0095085 | Spider monkey |
| | YDDLWQQWDNQVGNRKITPHNIATGTYPPKPQ | 61 | simian foamy |
| | KQYHINPKAKPSIQIVINDLLKQGVLRQSTSPMN | | virus |
| | TPVYPVPKPDGKWRMVLDYRAVNKTIPLIAAQ | | |
| | NQHSLGILTNLIRHKYKSTIDLSNGFWAHPITED | | |
| | SQWITAFTWEGKQHVWTRLPQGFLNSPALFTA | | |
| | DVVDILKEVPGVSVYVDDIYISSPTMEEHFQVL | | |
| | DSIFRKLLETGYIVSLKKSALARYEVNFLGFVISE | | |
| | TGRGLTSEFRERLQEITPPTTLKQLQSILGFLNFA | | |
| | RNFVPNFSELVQPLYQLISTASGNFIQWTAEHTL | | |
| | RLNELISALNHAGNLEQRRGDSPLVVKVNASDK | | |
| | TGYIRYYNDNSLIPIAYASHVESTAELKFTPLEK | | |
| | LLVTMHRALLKGIDLALGQPIKVYSPIASMQKL | | |
| | QKTPIPERKALSTRWVTWLSYLEDPRITFYYDK | | |
| | TLPDLKHVPASTDNNIITLLPITEYEAVFYTDGS | | |
| | AIKSPKTEQTHSAGMGIVMVVYTPEPNITQQWS | | |
| | IPLGDHTAQYAEISAVEFACKKASLLQGPVLIVT | | |
| | DSDYVARSANKELPFWRSNGFLNNKKKPLKHIS | | |
| | KWKNISDSLLLKRNITIVHEPGHQPSKTSIHTLG | | |
| | NSLADKLAVQGSYSVNTINKIPSLDAELNQILEG | | |
| | NLPKGYPKQYKYVLKNNELIVQRPEGDKIIPPK | | |
| | ADRLPLVKTAHELAHTGREATLLKLQTTHWWP | | |
| | NMRKDIITVLRQCKPCLQTDSTNLTPIPPVSQP | | |
| | TEKLPIQDYKDNIVKRADITKEEKGMLYKLLDK | | |
| | YDPLWQQWENQVGNRQITPHIIATGTINPKPQK | | |
| | QYHINPKAKPSIQIVINDLLKQGVLKQQNSIMNT | | |
| | PIYPVPKTEGKWRMVLDYRAVNKTIPLIAAQNQ | | |
| | HSAGILTNLVRQKYKSTIDLSNGFWAHPIDQDS | | |
| | QWITAFTWEGKQYVWTRLPQGFLNSPALFTAD | | |
| 8323 | VVDLLKEIPNVNVYVDDIYVSTETINQHFQVLD | YP_0095085 | Squirrel monkey |
| | KIFQKLLQAGYVVSLKKSNLCRYEVTFLGFTISK | 66 | simian foamy |
| | YGRGLTEEFQEKLRNISPPNSLKQLQSILGLLNF | | virus |
| | ARNFIPNFSELIKPLYELISTAQGQSISWEPKHSQ | | |
| | ALNNLIIALNHADNLEQRNGEVPLVIKINASNTT | | |
| | GYIRFYNKNGKRPIAYASHVFNHTEQKFTPVEK | | |
| | LLTTMHKAIIKGIDLAIGQPIEIYSPIVSMQKLQKI | | |
| | TLPERKALSTRWLSWLSYIEDPRFLFIYDKTLPD | | |
| | LKEMPPTQTDDYNPMLPLHQYLAVFYTDGSSIK | | |
| | SPDPTKTHSSGMGIVQAIYEPNFQIKHQWSIPLG | | |
| | DHTAQYAEIAAVEFACKKALQVTGPVLIVTDSD | | |
| | YVARSVNNELNFWRSNGFVNNKKKPLKHISKW | | |
| | KSISESLLLHKNITIVHEPGHQPSSTSVHTQGNAL | | |
| | ADKLAVQGSYTINNITIKPSLDTELRAVLEGKLP | | |
| | KGYPKNLKYEYNSPNLIVIRKEGQRIIPPLSDRPK | | |
| | LVKQAHELAHTGREATLLRLQNQYWWPKMRK | | |
| | DVSHCLRTCMPCLQTNSTNLTTTRPFQQI | | |
| 8324 | TIKIDIQKQQEQLLHTTNLSSEGKKYLKDLFIKY | NP_054716 | Equine foamy |
| | DNLWQKWENQVGHRRITPHKIATGTLNPKPQK | | virus |
| | QYRINPKAKADIQIVIDDLLKQGVLKQQTSPMN | | |
| | TPVYPVPKPDGRWRMVLDYRAVNKVTPAIATQ | | |
| | NCHSASLLNTLYRGQYKTTLDLANGFWAHPIQ | | |
| | ESDQWITSFTWNGKSYVWTTLPQGFLNSPALFT | | |
| | ADVVDLLKDIPNVEVYVDDVYFSNDTEEEHLK | | |
| | TMDLLFQKLQTAGYIVSLKKSKLGQHTVDFLGF | | |
| | QITQTGRGLTDSYKSKLLDITPPNTLKQLQSILG | | |
| | LLNFARNFIPNYSELITPLYQLIPLAKGIYIPWET | | |
| | KHTAILQKIIKELNASENLEQRKPDVELIVKVHV | | |
| | SPTAGYIKFANKGSIKPIAYHNVVFSKTELKFTIT | | |
| | EKVMTTIHKALLKAFDLAMGQPIWVYSPIHSMT | | |
| | RIQKTPLTERKALSIRWLKWQTYFEDPRLIFHYD | | |
| | DTLPDLQNLPQTTLGNEVDILPLSEYEVVFYTD | | |
| | GSSIKSPKKDKQHSAGMGIIAVRYQPQMNIIQE | | |
| | WSIPLGDHTAQFAEIAAFEFALKQAIRKMGPVLI | | |
| | VTDSDYVAKSYNQELDFWVSNGFVNNKKKPL | | |
| | KHVSKWKSIADCKKHKADIHVIHEPGHQNDLQ | | |
| | SPYAMGNNAADKLAVKASYTVFSVQTLPSLDA | | |
| | ELHQLLDKQTPNPKGYPSKYEYTLRDGQVYVK | | |
| | RTDGEKIIPSKDDRVKILELAHKGPGSGHLGKNT | | |
| | MYIKILNKYWWPNLIKDISKYIRTCTNCIITNTD | | |
| | NVPNKSYIVQE | | |
| 8325 | TIKIDVESQKHTLITESTLSPQGQMRLKKLLDQY | NP_044929 | Bovine foamy |
| | QALWQCWENQVGHRRIEPHKIATGALKPRPQK | | virus |
| | QYHINPRAKADIQIVIDDLLRQGVLRQQNSEMN | | |
| | TPVYPVPKADGRWRMVLDYREVNKVTPLVAT | | |
| | QNCHSASILNTLYRGPYKSTLDLANGFWAHPIK | | |
| | PEDYWITAFTWGGKTYCWTVLPQGFLNSPALF | | |
| | TADVVDILKDIPNVQVYVDDVYVSSATEQEHL | | |
| | DILETIFNRLSTAGYIVSLKKSKLAKETVEFLGFS | | |
| | ISQNGRGLTDSYKQKLMDLQPPTTLRQLQSILG | | |
| | LINFARNFLPNFAELVAPLYQLIPKAKGQCIPWT | | |
| | MDHTTQLKTIIQALNSTENLEERRPDVDLIMKV | | |
| | HISNTAGYIRFYNHGGQKPIAYNNALFTSTELKF | | |
| | TPTEKIMATIHKGLLKALDLSLGKEIHVYSAIAS | | |
| | MTKLQKTPLSERKALSIRWLKWQTYFEDPRIKF | | |
| | HHDATLPDLQNLPVPQQDTGKEMTILPLLHYEA | | |
| | IFYTDGSAIRSPKPNKTHSAGMGIIQAKFEPDFRI | | |
| | VHLWSFPLGDHTAQYAEIAAFEFAIRRATGIRGP | | |
| | VLIVTDSNYVAKSYNEELPYWESNGFVNNKKK | | |
| | TLKHISKWKAIAECKNLKADIHVIHEPGHQPAE | | |
| | ASPHAQGNALADKQAVSGSYKVFSNELKPSLD | | |
| | AELEQVLSTGRPNPQGYPNKYEYKLVNGLCYV | | |
| | DRRGEEGLKIIPPKADRVKLCQLAHDGPGSAHL | | |
| | GRSALLLKLQQKYWWPRMHIDASRIVLNCTVC | | |
| | AQTNSTNQKPRPPLVIP | | |
| 8326 | TIKLNLEEQQRTLLNNSILSKKGKEELKRLFEKY | QER92092 | Feline foamy |
| | NALWQSWENQVGHRKIRPHKIATGTVKPTPQK | | virus |
| | QYHINPKAKPDIQIVINDLLKQGVLIQKESTMNT | | |
| | PVYPVPKPNGHWRMVLNYRAVNKVTPLIAVQN | | |
| | QHSYGILGSLFKGKYKTTIDLSNGFWAHPIVPED | | |
| | YWITAFTWQGKQYCWTVLPQGFLNSPGLFTGD | | |
| | VVDLLQGIPNVEVYVDDVYISHDSEKEHLEYLE | | |
| | ILFNRLNEAGYIVSLKKSNIANSSVDFLGFQITNE | | |
| | GQGLTDTFKEKLENITAPTTLKQLQSILGLLNFA | | |
| | RNFIPDFTELIAPLYALIPKSTKNYVPWQIEHSTT | | |
| | LETLIAKLNEAKYLQGRRGDKTLIMKVNASYTT | | |
| | GYIRYYNEGEKKPISYVSIVFSKTELKFTKLEKL | | |
| | LTTVHKGLLKALDLSMGQNIHVYSPIVSMQNIQ | | |
| | KTPQTAKKALASRWLSWLSYLEDPRIRFFYDPQ | | |
| | MPALKDLPAVNIGENNKKHPSNFQHIFYTDGSA | | |
| | ITSPTKEGHLNAGMGIVYFINRDGNLQKQQEWS | | |
| | ISLGNHTAQFAKIAAFEFALKKCLPLGGNILVVT | | |
| | DSNYVAKAYNEELDVWASNGFVNNRKKPLKHI | | |
| | SKWKSVANFKKLRPNVVVTHEPGHQKLNSSPH | | |
| | AYGNNLADQLATQASFKVQHDKNSKLDTEQIK | | |
| | AIQARQNNERVPVGYPKQYTYELRNNKCMVLR | | |
| | KDGWREIPPSRERYKLIKEAHDISHASREAVLLK | | |
| | IQENYWWPKMKKDVSSFLSTCNVCKMVNPLNL | | |
| | KPISPQAIV | | |
| 8327 | DIEKLIQNQIDKTNINKDKLRELLLNKREILAKSL | XP_0078891 | Callorhinchus |
| | TDCGKVKSNAEIRGKLHHKQRQYKIRREDKETI | 23 | milii |
| | GKIINNLMEQGVIKKCRSPTNSPIFLKKKPDGSW | | |
| | RLLLDCKALNECTNPKQGQSISSHGSIEKLTREK | | |
| | YHTTLNIANGFWSIPIVEKDQFKTAFTYQGQQY | | |
| | QWTRLPEGWCNSTVIFNEAIRRVLDDNPKITRF | | |
| | GGVIHFSNDNAESHIKLLKQILEKLDNHGLKIDL | | |
| | RKSQIGRYAVDFLGHQISETEDRLSRKFKEEVSQ | | |
| | VKPPKTRKELQSVLGKFNFAREFVINYAGKAAS | | |
| | LFKLTNKEPFQWDTNAQECLNQLQQDIQKAVP | | |
| | HSTRKPKSSLILDIYVSKDSSVTAVKQKSVTGEE | | |
| | TLKYLSFNFSKAEKKFAKDERLLATLFCTLKQT | | |
| | CQMVTSKEKITVRTSYPELAGVTRESQRNHKAL | | |
| | KCRWKKWESLLYDQRISFELRNGIEENE | | |
| 8328 | VDVPAVTKQKIEESDFSPAGKKKLREIIASAKVA | XP_0149148 | Poecilia latipinna |
| | RFKNDCGDLGPRFVHHIEGGVHPPVRQYPLNPG | 25 | |
| | AVEEMDKIVKELGSLGIIREELNPITNSPIQAVKK | | |
| | PESAGGGWRPVINFKALNRRTIANRASLINPQGT | | |
| | LKTLRVKKFKSCIDLANGFFSLRLARQSQGKTA | | |
| | FTHKGKSYVWQRLPQGYKNSPNVFQSAVMEVL | | |
| | GDVGATVYIDDVFIADDTEEEHLERLRKVIENL | | |
| | TKAGLKLNLKKCQFGQFQVNYLGFQVTSDLGL | | |
| | SDGYREKMMNIQPPQSENELQKILGLCNYVRD | | |
| | HVPNYQKYAKPLYNCLKKSVENEGGGKRPWV | | |
| | WTAANQRDLEDLKKAIQAAVRLEPRSLSDRLV | | |
| | AEINCEEDDAMIKVSNENGGLVTLWSYTLTSVE | | |
| | KKYPQEEKELAVLARYWSVLKDLAQGQPVKVI | | |
| | TQSQVHKYLRKGTVESTKATNARWGRWEDILL | | |
| | DPELEIGPAQPTNKKRQQETPEKPGQPEWTLYT | | |
| | DGSKKESDQVAYWGFILKQDGKERCRQKGKAP | | |
| | GSAQAGEVTAILEGLLELGKRKIKSARLVTDSY | | |
| | YCAQALKEDLAIWEENGFETAKGKPVAHRDLW | | |
| | KKIAELKMQLELEVEHQRAHTHEGAHWRGND | | |
| | EVDCYVQQRKIVFVGIEKWDSTPRGREVPEEYV | | |
| | DEVVRSVHEALGHAGVRPTRKELEEHELWIPV | | |
| | KQVQRVLRDCEVCGKYNAGRRGQRLEGLTI | | |
| 8329 | VNIPEATEQKLEESDFSPEGKEKLRKIITRATVA | KAE8297773 | Larimichthys |
| | RFKNDCGDLGSKYVHTIEGGVHPPVRQYPLNPG | | crocea |
| | AVEEMDKIVTELSALDIIREEPNPITNSPIQAVKK | | |
| | PEAAGGGWRPVINFKALNRRTVANRASLINPQG | | |
| | ALKTLQVKRFKSCIDLANGFFSLRLAKQSQGKT | | |
| | AFTHKGKAYVWQRLPQGYKNSPNVFQAAVMD | | |
| | VLKDLGVTIYIDDVFLADDTEEEHLQRLRQVVE | | |
| | RLTEAGLKLNLKKCQFGQFRVNYLGFQVAADL | | |
| | GLSDGYREKLNQVRPPTSENDLQKILGLCNYVR | | |
| | DHVPNYQKYAKPLYACLKKKGEESEEETPKKW | | |
| | SWTATDQQNLGRLKAVIQDAIRLEPRSLTTRLV | | |
| | AEVSCEDDDAVVKVKNEGGGMVTLWSYTLSS | | |
| | VERKFPQEEKELAVLARYWGTMKDLAQGQGIK | | |
| | VITQSQVHRYLRKETIESTKATNTRWGRWEDIL | | |
| | LDPDLEIGPAQPANRKAQKPQETEEKSYEWILY | | |
| | TDGSRKGQDDTAYWGYILKQDGKEQFRQKGR | | |
| | VSGSAQAGEVTAILEGLLELEKRKVKTARIITDS | | |
| | YYCAQALKEDLTIWEENGYEGAKGKMVAHQD | | |
| | LWKKIAELRLKMCLDVVHQKAHGKEGAHWKG | | |
| | NDEVDRYVQQRRIVFVGREKWEQTPKGRVVPE | | |
| | SSVVEVVQAVHEALGHVGTMSTRKELEKQQL | | |
| | WIPVGRVRQVLKDCNVCGRYNAGRRGKRVDG | | |
| | LTI | | |
| 8330 | VNIPEATEQKLEESDFSHEGKEKLRKIITRATVA | KAE8289514 | Larimichthys |
| | RFKNDCGDLGSKYVHTIEGGVHPPAVKKPEAA | | crocea |
| | GGGWRPVINFKALNRRTVANRASLINPQGALKT | | |
| | LQVKRFKSCIDLANGFFSLRLAKQSQGKTAFTH | | |
| | KGKAYVWQRLPQGYKNSPNVFQAAVMDILKD | | |
| | LGVTIYIDDVFLADDTEEEHLQRLRQVVERLTE | | |
| | AGLKLNLKKCQFGQFRVNYLGFQVAADLGLSD | | |
| | GYREKLNQVRPPASENDLQKILGLCNYVRDHV | | |
| | PNYQKYAKPLYACLKKKGEESEEGTPKKWSWT | | |
| | ATDQQNLGRLKAVIQDAIRLEPRSLGVAEVSCE | | |
| | DDDAVVKVKNEGGGMVTLWSYTLSSVEKKFP | | |
| | QEEKELAVLARYWGTMKDLAQGQGIKVITQSQ | | |
| | VHRYLRKETIESTKATNTRWGRWEDILLDPDLE | | |
| | IGPAQPANRKAQKPQETEEKSYEWILYTDGSRK | | |
| | GQDDTAYWGYILKQNGKEQFRQKGLVSGSAQ | | |
| | AGEVTAILEGLLELEKRKVKTARIITDSYYCAQA | | |
| | LKEDLTIWEENGYEGAKGKMVAHQDLWKKIA | | |
| | ELRLKMCLDVVHQKAHGKEGAHWKGNDEVD | | |
| | RYVQQRRIVFVGREKWEQTPKGRVVPESSVVE | | |
| | VVQAVHEALGHVGTMPTRKELEKQQLWIPVGR | | |
| | VHQVLKDCDVCGRYNAGRRGRRVDGLTI | | |
| 8331 | VKKFKSCIYLANRFFSLRLAKQSQGKTAFTHKG | KAF0022147 | Scophthalmus |
| | KAYVWQRLPQGYKNSPNVFQSAVMDVLSGLG | | maximus |
| | ATVYIDDVFIADDTEEEHLERLQKVVERISAAGL | | |
| | KLNLKKCQFGQFQVNYLGFQVAMDLGLSDGY | | |
| | REKINQITPPTTLNELQKILGLCNYVRDHVPGYQ | | |
| | QYAKPLYACLKTKEVLRNGKPDRNWNWTATD | | |
| | QDNLRKLKDAIQQAVRLEPRSLTTKLVAEVSCE | | |
| | EEDAVLRVSNEGGGLVTLWSYTLSSVEKKYPPE | | |
| | EKELAVLAKYWGALKDLAQGQTIKVTTRSQVH | | |
| | RFLRKGTVESTKATNTRWGRWEDILLDPDLEIS | | |
| | PEKLPSKKTTKEETTDKKPYEWTLYTDGSKKG | | |
| | QDDNAYWGFILKLNEKESFRQRGRALGSAQAG | | |
| | EVTAIMEGLLELGKRKIKRVRIITDSFYCAQALQ | | |
| | EDLTIWEENGFESAKGKMVAHQDTHWRGNNE | | |
| | VDRYVQQRKVVIVGIEKWDKTPKGRVVPEEFV | | |
| | KEVVQAVHEALGHAGTIPTRKELEKQDLWIPEK | | |
| | QIRRILKDCETCGKFNAGRRGQRVDGQTI | | |
| 8332 | MKEVLIGANLAIGKNDTGLISKRFQHTIHGGQH | GCB70404 | Scyliorhinus |
| | RPQKQYPLTRGAKSELEIAIKELEQQGIIQKVTY | | torazame |
| | ALTNSSLQVVSKPDGTFRMITNYKALNKVTKK | | |
| | DKRYLINPQTTLEQVAGKIYLTSIDLANGFWSVP | | |
| | LDPDSREKTAFTFGTKHYVYCRLPQGYVNSPNH | | |
| | FQAIVRELMKDDLALVYIDDVLIGDDDQDKHV | | |
| | ERVARIIKTLSEAGFKIGLKKCQIGRSEVNYLGY | | |
| | SVSKEGREACIEMRKKVAEITAPVSRKGVKKIM | | |
| | GILGYLRPVVKDFSLYAKPIYETLKGDFIWSSEA | | |
| | QGGLDQLKTAVAISGPLVGRQEEDDLSIKLDIY | | |
| | KNGYGMVLVNHSNQTLIKHLTGIWPRAEQKCS | | |
| | DIEKCLAAII | | |
| 8333 | VDLEHEINRLVKETLLPKNELRKLLEKYRNSFA | KAG1925097 | Pimephales |
| | KSKNDCGKLDDKYEYTILGGIPAPQRQYPLNKA | | promelas |
| | AYPEIRGTLNELLRKGIISVGENCPTNAPIQAVIK | | |
| | IDGSYRLVCNFKALNRLTVPDTRYLINARDATN | | |
| | CLDDGKILSKIDLANGFWSVPLAKDSRARTAFT | | |
| | FEGKQYVFNRLPMGFCNAPNAFQAIILEILEGLP | | |
| | VTAYIDDVLIATQTTEEHMRVLSETIRKLADAG | | |
| | FLLNLKKLELGKENVNFLGFEISGAERGIAKSTQ | | |
| | EKLEELKKERITNLRQLQSLLGRLNFVRDLIPGY | | |
| | SAKAKILYKATAGKDFHWDDRLESIKCDIINMA | | |
| | LASGRIVRRNPDKNLRVKIDNTPEEMELILYNEG | | |
| | DTKSPVMFISHEKPANHKKHENMSPVDILATIA | | |
| | RNLLVIKALAAEKLIIIVAKGEGIDLLAREAKNL | | |
| | VNENKRVHVFTWSKWIKIIDDNQFEFRNEKSPK | | |
| | KDRRTVIDPEQICYFTDGSSEKGETWWGFMVK | | |
| | LKGKIIHKEKGRLDNDKSAQEAEVTAVAKAIM | | |
| | HMRDNNRKKCVLVTDSEYVYLGIVQNLSTWEQ | | |
| | NNFNNAKGKPLAQVELWKVISECAKVVQPRVL | | |
| | HQSSHTVQKTPAAVGNREVDQYVRVRAITKES | | |
| | EGNLLQELHDKLNHPSTTYVAKYCKQLGLHVQ | | |
| | NLKTSYQKIKIKCPDCRKVMSSVHHDFGHI | | |
| 8334 | FPVYKAEEEENEEIPDEISRLLEQERKTIQPYGDE | ABE77575 | Medicago |
| | LEVINLGTKEDKKEIKVGASLETSVKKQVIELLK | | truncatula |
| | EYVDVFAWSYRDMPGLDTDIVVHHLPLKPECP | | |
| | PVKQKLRRTRPDMALKIKEEVQKQIDAGFLVTS | | |
| | NYPQWLANIVPVPKKDGKVRMCVDYRDLNKA | | |
| | SPKDDFPLPHIDVLVDSTAKSKVFSFMDGFFGY | | |
| | NQIKMAPEDREKTSFITPWGTFCYKVMPFGLIN | | |
| | AGATYQRGMTTLFHDMIHKEIEVYVDDMIVKSI | | |
| | TEEDHVKYLQKMFQRLRKYKLRLNPNKCTFGV | | |
| | RSGKLLGFIVSQKGIEVDPDKVKAIREMPAPRTE | | |
| | KEVRGFLGRLNYISRFISHMTATCGPIFKLLRKE | | |
| | QGIVWTEDCQKAFDNIKKYLLEPPILIPPIEGRPL | | |
| | IMYLTVLENSMGCVLGQQDETGRKEHAIYYLS | | |
| | KKFTECESRYSILEKTCCALAWAAKRLRHYMIN | | |
| | HTTWLVSKMDPIKYIFEKPALTGRIARWQMLLS | | |
| | EYDIEYRSQKAIKGSILADHLAHQPLED | | |
| 8335 | FSSVENRREEQILFDVVNIPYNYNAIFGRATLNK | ABF96966 | Oryza sativa |
| | FKAISHHNYLKLKMPGPKGVIVVKGLQPSAASK | | Japonica Group |
| | RDLAIINRAVHNVETEPHERPKHTPKPTPHGKV | | |
| | AKVQIDDFDPTKLVSLRSPRLKLRKMSADRQEA | | |
| | AKAEIHMNPLNIPKTSFVTPFGTFCQLRMPFGLR | | |
| | NAGATFARLVYKVLGKQLGRNVKAYVDDIVV | | |
| | KIHKAFDHANNLQETFDSLRAAGIKLNPEKCVF | | |
| | SVRAGKLLGFLVSERGIEANPEKIDAIQQMKPPS | | |
| | SVHEVQKLAGRIAALSRFLSKAAERGLPFFKTL | | |
| | RGAGKFNWTPECQAAFDKLKQYLQSPPVLISPP | | |
| | LGSELLLYLAASPVAVSAALVQETESGQKPVYF | | |
| | VSEALQGAKTRYIEMEKLAYALVMASRKLKHY | | |
| | FQAHKVIVPSQYPLGEILRGKEVTGRLSKWAAE | | |
| | LSPFDLHFVARSAIKSQVLADTTEY | | |
| 8336 | VNPLSAPVLREEIAALLAKGAIEPVPPAEMESGF | XP_689703 | Danio rerio |
| | YSPYFIVPKKSGGSRPILDLRVLNRCLHKLPFRM | | |
| | LTQRRILQCVRPRDWFAAIDLKDAYFHVSILPR | | |
| | HRQFLRFAFEGRAWQYKVLPFGLSLSPRVFTKL | | |
| | AEGALAPLRLAGIRILSYLDDWLILAHSREQLIV | | |
| | HRDEVLRHLRLLGLQVNREKSKLAPVQRISFLG | | |
| | MELDSITMRLLGHMASAAAVTPLGLLHMRPLQ | | |
| | HWLHDRVPRRAWHAGTHRVSVTALCRRALSP | | |
| | WNDPSFLQAGVPLGQASSHVVVSTDASNTGWG | | |
| | AVCRGHAAAGLWKGAQLHWHINRLELLAVFL | | |
| | ALHRFLPVLERQHVLVRTDSTAAAAYINRMGG | | |
| | MRSRRMSQLARRLLLWSHPRLKSLRAIHVPGTL | | |
| | NRAADALSRQLLR | | |
| 8337 | NTEELEKLLADYPAEARVFCALEPKRDGTSRPII | WP_0156413 | Candidatus |
| | KPNKPLNQWLKRMKRALYRQRRDWPTFIHGG | 29.1 | Saccharimonas |
| | VKKRSYVSFARPHANKNTVITIDIKDCFGSITQS | | aalborgensis |
| | EVQQALVSKLGLPDGLASRLAAKLCYKRRIPQG | | |
| | FATSSYLTNLYLNDTLLKINRQLKRKQIDMTVY | | |
| | VDDIALSGQKVDSAVIINLVTLELSRARLAISKA | | |
| | KVKVMRSHSPQIICGLVVNKGVALSRQKRKEIF | | |
| | SDIA | | |
| 8338 | FHITSKKRLAILLHSSVKELNNIVRSKDQMYQYF | WP_0004460 | Acinetobacter |
| | NETQTDNSGNIIKVRPIQNPHDRLKQIHSRIGKFL | 53 | baumannii |
| | GNLKAPEYLHSKRSKSAISNAKAHVGIKGHTLN | | |
| | IDITDFYPSTSRAKVQAFFGYTLQYPTDIAKYLS | | |
| | EICTVKNCLPTGSPLSSALAFWANKSMFDEIYR | | |
| | VAKSRAITMTVYVDDISFTGRAVNQNFLKKIIQI | | |
| | VGKYQHKIKQEKIKFFPEYSVKFVTGVAILNGR | | |
| | LQPAHRHYRDIRVLQK | | |
| 8339 | ARKENYYKAFDHASKNKHGKKAIIKFEADLEK | WP_0119662 | Parabacteroides |
| | NLSDLLYSFENGTFVTSPYRFMTVHEPKKRLIG | 32.1 | distasonis |
| | MLPFPDHVQHWAMLNEVEDYFTRSFSAYTYGG | | |
| | VKGRGPHAYMRMIRKVLRKYPERTTDYLLCDI | | |
| | HHFYPTVNHPVLKSQLRTRIKDNHLLRRLDEIID | | |
| | SVEGDTGMFPGTKLAQFFSLVYLYLFDHDLKRC | | |
| | FHVGECPALVEYYTKRYIEESIATAKTEHDYEEL | | |
| | SKGIQYLSDRFKGYLNRLDFCYRLADDVLILHE | | |
| | DTVFLHLVIEWIGLYYANELRIGLNPRWK | | |
| 8340 | FSDSLPPIFSSEELSLKESNINVSKDYLSKNGSNK | WP_0914746 | Aliicoccus |
| | RSKLINFSIPKNSSFRRTLSIVHPLHYIKFANLIDE | 05.1 | persicus |
| | QWENISKHFEKSSVSLTKIIKNNSKLEREHGFEA | | |
| | MRYKQIENLSLNRFILKIDINRYYPSIYTHSIPWA | | |
| | LHGKKYSKLNISEENLGNNLDTLTRNMQDGQTI | | |
| | GIPIGPFSSDIIQEIIGTAIDEDFSKKMEYKVPGYR | | |
| | YTDDMEYYFKNLNEANNALSVMNNVLKNYEL | | |
| | DLNSEKTVIEKIPMVLEKEWIRSLKNFRENKNYS | | |
| | KKNVRKEKELLIEYFNSIFN | | |
| 8341 | VPPALWGSRHNQRRFLRNVKKFISLGKHAKLSP | XP_0047684 | Mustela putorius |
| | QELTWKMKVQDCAWLRGSPGACSVPAAEHRR | 47 | furo |
| | REGVLARLLCWLMGTYVVELLRSFFYVTETTF | | |
| | QKNRLFFYRKSVWSPLQTLGVRQHCTSVRLREL | | |
| | SAAEVRRQHEARATLLTSRLRFLPKPGGLRPIVN | | |
| | MDYVAGARALCRDKKIQHLTSQVKTLFSVLNY | | |
| | ERARRPRLLGASVLGMDDIHRAWHDFVLRVRA | | |
| | QDPAPRLYFVKVDVTGAYDALPQDRLAEVVAN | | |
| | VLRPHENTYCLRRYAVVRRTAQGHVRRSFKRH | | |
| | VSTFTDLPPYMRQFVERLQETTSLRDSVVIEQSY | | |
| | SLNEASSGLFQLFLSLVYSHVIRIGGNLLLRLVD | | |
| | DFLLITPHLKRAQAFLRTLVRGVPEYGCSANLQ | | |
| | KTAVNFPVEDMALGSTAPLQLPAHCLFPWCGL | | |
| | LLDTQTLEVS | | |
| 8342 | LDLKVIKPSKSPHMAPAFLVNNEAEKRRGKKR | NP_056728 | Cauliflower |
| | MVVNYKAMNKATVGDAYNLPNKDELLTLIRG | | mosaic virus |
| | KKIFSSFDCKSGFWQVLLDQESRPLTAFTCPQG | | |
| | HYEWNVVPFGLKQAPSIFQRHMDEAFRVFRKF | | |
| | CCVYVDDILVFSNNEEDHLLHVAMILQKCNQH | | |
| | GIILSKKKAQLFKKKINFLGLEIDEGTHKPQGHIL | | |
| | EHINKFPDTLEDKKQLQRFLGILTYASDYIPKLA | | |
| | QIRKPLQAKLKENVPWRWTKEDTLYMQKVKK | | |
| | NLQGFPPLHHPLPEEKLIIETDASDDYWGGMLK | | |
| | AIKINEGTNTELICRYASGSFKAAEKNYHSNDKE | | |
| | TLAVINTIKKFSIYLTPVHFLIRTDNTHFKSFVNL | | |
| | NYKGDSKLGRNIRWQAWLSHYSFDVEHIKGTD | | |
| | NHFADFLSREFNKVNSSGGS | | |
| 8090 | QHYHDTIQQENQLIPSFFTYIAKLKQDLDSLPDE | XP_0012490 | Coccidioides |
| | FFHKRRSKVKDYPQTKKTPVKPLPKISEEEHKQ | 02 | immitis RS |
| | QIDEELCLRCGQPGHKTKFCTNSSNKSQQTDKK | | |
| | NKNQAKTRTAKPMQDPGQTLERQGVNPIKKAS | | |
| | RCKQAALLDSGTTVNSISYKLASQLDWDQPETP | | |
| | MEVIEMLNRAEADWYSIYKTQLTITDSMGTIKM | | |
| | KKYYCPSRQRFYKNDILIFSASEKEHEKHVRLV | | |
| | MEYLREYQLFAKLAKCAFKRQTISYLGYIIDNE | | |
| | GIKMDPKQIQVITEWLLLQSFHNIQIFLGFANFY | | |
| | QRFIQKYSVIVALLTDLLKSSEKRRKKEPFLLTP | | |
| | TTRKVFCELQAVFFREPVIQHYNPECRIHLETDT | | |
| | SEHAAEMVQGRPGGTKIIDVLNLLLEAQGDDSS | | |
| | VQARFTKTESQQSE | | |
| 8343 | AASSLSTLQHVTLQKLNKLDSQRQQFESDKKSI | EAT92517_1 | Parastagonospor |
| | LEQVSSVPDHRSKVEALLDGFELHGIAPKQADL | | a nodorum SN15 |
| | SISNLKHFVHQAKHDPSVSASLLKDWQSRLEHE | | |
| | LNVKSNKYEYAALFGKLVTEWIKHSTLVKSAD | | |
| | VSDGSIAKGRKKMQEQRQSWENYAFVEKEVN | | |
| | QSTIEQYLSDIFGDALQTEKIKKSPLRVLRDSMK | | |
| | EVMDFKSDLDTSEKDFSSNKRFGHSAPHGSRFTI | | |
| | ELLQSCIRGVKKADLFTGRKLEMIIDLEKQPAVL | | |
| | KELVDVLNMDVDGLDHWEWDGPVPLNMRRQ | | |
| | TNEIHQAILLHFIGKTWAVALKKAFTNFYHSGA | | |
| | WLQAPYRSMPKKIRQRREHFIENSNKSGDSVRN | | |
| | YRRQKYQQEYFMTQLPSNAFEDAREYDAAEGQ | | |
| | EKNSHIATKQTMLRLLTTEILLNTKVYGECSVL | | |
| | QSDFKWFGPSLPHDTIFAVLEFFGVPAKWLRFF | | |
| | KRFLEVPVVFAQDGAGAKARVRKCGIPNSHILS | | |
| | DALGEAVLFCLDFAVNRRTKGANIHRFHDDLW | | |
| | FWGQETTSVQAWEAIKEFTEVMGLQLNEEKTG | | |
| | SSIIVADKSRARVPHPNLPEGNLHWGFLELDAS | | |
| | AGRWVIDRAQVDEHITELRRQLDACHSVMAWI | | |
| | QAWNSYVGLFFNTNFAQPANCFGRQHNDMIIE | | |
| | TFSHIQRSFFGKYGTANVTEYLRSVLKERFQTTD | | |
| | AVPDAFFYFPVELGGLGLNNPSISAFATYQNSSR | | |
| | DPSARIERAFEEEREAYDTAKQRWDAGDVPCP | | |
| | NRETDEPFMSFEEYTAFREETSHPLFEAYMNLL | | |
| | ECPVEERVETSDEMYEALRRSDAPHALGSNHY | | |
| | WLWIFNLYAGDLKQRFGGQGVQLGERDLLPVG | | |
| | LVEVLKSEKIYPGSNPFGINQLFTLFRLRKKLRM | | |
| | SVANGWGGYDVTKEPRRTNKDEL | | |
| 8344 | PSTQTEFEKQLQQMLLDADALQSDEERVKLRT | XP_0225179 | Astyanax |
| | VLTKYRASFSQDSMDCGLTHIHMIRIPTHPDAAP | 07 | mexicanus |
| | AYVRQYKIPLASYGPVQEIIDDLLDKGIIRPCNST | | |
| | YSAPLWPVLKPNGKWRLTIDYRRLNDQVPLSR | | |
| | WPMTQLEQELPRVRDAKYFSTLDVASGFWTIP | | |
| | VHVEDQHKLAFTFAGRQFTFTRCPFGYSNSPAE | | |
| | FNIFLNKACPDARERGTLIYVDDVLIRNNSLDAH | | |
| | LEEIDHVLDQLTKAGAKISLAKCQWCKTKVNY | | |
| | VGLLVGPDGVLPQPCRAQGIVDIAEPKTIHALRS | | |
| | FLGVCNYSRQFIENFAELAKPLYQLLKQDVPFI | | |
| | WGEAQAQAMQTLKDKLASAPCLTYPDHSREFY | | |
| | LQVGFSEHCVSAGLYQVHDRDKRVVAYASKAL | | |
| | MAPELKYSDCEKALLATVWAVKHFSNYLGGQ | | |
| | KIIVETNHQPVVFLNSQRIREGVVTNARVASWL | | |
| | MALQSFEVEVRYAQNSRLPLGTDLAACQRCET | | |
| | DIPATSVPIPNLASQKPTNHRYFDPKECENIPTVY | | |
| | VDGCSFRHDQEGLKAGAGIVWLDDNPCEPQQF | | |
| | KLGSQTSQYAEIAAILITIQLAIDQGVKTLVICTD | | |
| | SNYARLSFTCHLPIWKTKGFLTSGRKAVKHTEL | | |
| | FTAADYLVVRHDMLVYWKKVRGHSRVPGTDK | | |
| | TYNDQADSLAKRGALEGVSWVFDPLKYPTQPN | | |
| | PTVLAVTRAQAKQTSTTEIPPCAAVSIDPEITDA | | |
| | DLITLQDADPDIKSIKAFLLDPTNNPITSQMLEAS | | |
| | IPLKQLLDNRAFLKVVKGLLVHVTETHTSPAFV | | |
| | VPPCHRGVMLGHAHDSPSAGHKGIKETYRTLK | | |
| | QVAFWPRMREHVASYIKGCLVCCQFQPANPLH | | |
| | RAPLQRK | | |

TABLE 12
Exemplary ancestral sequence reconstruction (ASR) RT domains

| SEQ | | | |
|---|---|---|---|
| ID | | | |
| NO: | Sequence | Length | Name |
| 8345 | QKEPTQDVTLPQTWLTDFPQAWAETAGMGLAVQQAPLVIE | 478 | N43.ZFERV |
| | LKATATPVSIKQYPMSREARRGIKPHIQRLLDQGILVPCQSP | | |
| | WNTPLLPVKKPGTNDYRPVQDLREVNKRVEDIHPTVPNPYN | | |
| | LLSGLPPERQWYTVLDLKDAFFCLRLHPTSQPLFAFEWRDP | | |
| | ETGMSGQLTWTRLPQGFKNSPTIFDEALHRDLADFRIQHPD | | |
| | VTLLQYVDDLLLAADTEQDCLKGTQALLQTLGELGYRASA | | |
| | KKAQICQREVTYLGYKLKGGQRWLTEARKETVLQIPTPKTA | | |
| | RQVREFLGTAGFCRLWIPGFAELAAPLYPLTKEGSAFNWGE | | |
| | KEEKAFQELKQALLTAPALGLPDLTKPFQLFVDEKQGIAKG | | |
| | VLTQKLGPWRRPVAYLSKKLDPVAAGWPPCLRMVAATAL | | |
| | LTKDAGKLTMGQEIVILTPHAVEAVLKQPPDRWLSNARMIH | | |
| | YQALLLDTPRVQFHPTVALNPATTEPLPES | | |
| 8346 | SKEPKQDVTLPQTWLSDFPQAWAETAGMGLAVQQPPIVIQL | 478 | N42.ZFERV |
| | KATATPVRIKQYPMSREARRGIKPHIQRLLDLGILVPCQSPW | | |
| | NTPLLPVKKPGTNDYRPVQDLREVNKRVEDIHPTVPNPYNL | | |
| | LSSLPPERQWYTVLDLKDAFFCLPLHPESQPLFAFEWRDPET | | |
| | GRSGQLTWTRLPQGFKNSPTIFDEALHRDLADFRIQHPDLTL | | |
| | LQYVDDLLLAADTEQDCLKGTQALLQTLGELGYRASAKKA | | |
| | QLCQREVTYLGYKLKGGQRWLTEARKETVLQIPTPKTPRQV | | |
| | REFLGTAGFCRLWIPGFAELAAPLYPLTKEGNAFNWGEKEE | | |
| | KAFQELKQALLQAPALGLPDLTKPFQLFVDEKQGIAKGVLT | | |
| | QKLGPWKRPVAYLSKKLDPVAAGWPPCLRMIAATALLTKD | | |
| | AGKLTLGQEIVILTPHAVEAVLKQPPDRWLSNARMIHYQAL | | |
| | LLDTPRVQFDPSVALNPATTETLPES | | |
| 8347 | LTQGDIQEINPEVWATEGKYGCLDIPPIKIEMQKDTPAIRVK | 446 | N30.ZFERV |
| | QYPMSPEGKKGLASVIEHLLKENILEPCMSPHNTPILAIKKDE | | |
| | GKFRLVQDLREINKRTIARHPVVPNPYTLLSKIPREHTWFTVI | | |
| | DLKDAFWACPLAEECRDWFAFEWEHPDRGRKQQLRWTRL | | |
| | PQGYTESPNIFGQALETLLEQFSPKEGVQILQYVDDLLISGET | | |
| | EKEVKDVSIQLLNFFGEKGLKVSQSKLQFVETEVTYLGHIIG | | |
| | KGSKRLSPARISGIVSISPPKTKRDIRKLLGLFGYCKHWIDKY | | |
| | TQGVKFLYDKLIDQEPMNWTESDEKQLQDLKEKLSSAPVLS | | |
| | LPDLKKEFDLFVNTEEGIAYGVLTQEWGGYRKPVAFLSKLL | | |
| | DPVARGWPACLQAVAAAAILIEEAQKLTLQGKIPHDLKTILS | | |
| | QRAQDNLELTTSPHQRDRTLTFKRNKR | | |
| 8348 | LTQEDIEEINPEVWAEEGKSGLLDIPPIKIEMQKETPPIRVKQ | 468 | N22.ZFERV |
| | YPISPEGRKGLAPIIEQLLKEGILEPCMSPHNTPILAVKKAEG | | |
| | KYRLVQDLREINKRTVTRHPVVPNPYTLLSQIPREHAWFTII | | |
| | DLKDAFWACPLAEECRDWFAFEWEHPETKRKQQLRWTRLP | | |
| | QGFTESPNLFGQALEKLLEQFSPEEGVQILQYVDDLLISGED | | |
| | QSEVRETSIQLLNFLGEKGLKVSKSKLQFVESEVTYLGHLIG | | |
| | KGYKRLSPERIAGILSIPPPKTKRDIRKLLGLFGYCRLWLDKY | | |
| | TQSVKFLYDKLVDSEPIEWTEEDEKQLKDLKEKLSSAPVLSL | | |
| | PDLKKEFDLFVNTEEGVAYGVLTQEWGGCKKPVAFLSKLL | | |
| | DPVARGWPTCLQAVAAAAILIEETQKLTLQGKIRVHTPHDL | | |
| | KTILSQKAQKWLTDSRILRYEIALMNTDNLEFTTSPIQRDRT | | |
| | LTFKRNKK | | |
| 8349 | LTLEDEEKINPEVWYTPDSVGRLDIEPITVTIKDPDTPIRIKQY | 644 | N8.ZFERV |
| | PISLEGRRGLKPVIERLLSKGLLEPCMSPHNTPILPVKKPDGS | | |
| | YRLVQDLREINKRTVTRFPVVANPYTLLSKLSPENQWYSVI | | |
| | DLKDAFWACPLDEESRDYFAFEWEDPETHRKQQLRWTVLP | | |
| | QGFTESPNLFGQALEQLLQEYQTGEGVTLIQYVDDLLIAGET | | |
| | EEEVRKESIKLLNFLGLKGLKVSKAKLQFVEEEVKYLGHWL | | |
| | SKGEKKLDPERVKGILSLPPPKSKRQIRQLLGLLGYCRQWIE | | |
| | NYSSKVKFLYEKLSQGGLVKWTEEDEKQLKRLRQDLIQAP | | |
| | VLSLPDLKRPFYLFVNTDNGTAYGVLTQEWAGKKKPVGYL | | |
| | SKLLDPVSKGWPTCLQAVVACALLTEEAHKITFNSELKVLS | | |
| | PHNIRGILQQKADKWITDSRLLKYEGILLDSPKLTLEVTGLQ | | |
| | NPAQFLYDEKPVAHNCMATIEEQTKIRPDLEEEELETGERLF | | |
| | VDGSSRVIEGKRVSGYAIIGGPEVIESGPLNKTWSAQACELY | | |
| | AVLRALERLKDKEGTIYTDSKYAFGVVHTFGKIWENAADQ | | |
| | EAKKAALTESEQKLKALFLPKRLSIIHCPGHQKGHSAEARG | | |
| | NRMADQAARKAAITETPDTSTLL | | |
| 8350 | LTQEDEEKINPEVWHTEDEAGRLDIEPISIEIERPEDPIRIKQY | 644 | N7.ZFERV |
| | PISLEGRRGLKPIIERLLKKGILEPCMSPHNTPILPVKKPDGSY | | |
| | RLVQDLREINKRTVTRFPVVANPYTLLSRVSPENQWYSVIDL | | |
| | KDAFWACPLAEESRDYFAFEWEDPETNRKQQLRWTRLPQG | | |
| | FTESPNLFGQALEQLLQQFSPGEGVTILQYVDDLLIAGETEEE | | |
| | VREATIKLLNFLGEKGLKVSKSKLQFVEPEVKYLGHWISKG | | |
| | KKRLDPERVAGILSLPPPKSKRQIRQLLGLLGYCRQWIENYS | | |
| | QKVKFLYEKLTEGGKIKWTEEDEKQLKRLKQALITAPVLSL | | |
| | PDLKKPFHLFVNTDNGTAYGVLTQEWAGVKKPVGYLSKLL | | |
| | DPVSRGWPTCLQAIVAVALLIEEAQKITFGGELIVYTPHNVR | | |
| | TILQQKAEKWLTDSRLLKYEAILLNAPKLELRVTKLONPAEF | | |
| | LYLEKPVSHNCTDTIEEQTKVRPDLEDEELEEGEKWFVDGS | | |
| | SRVIEGKRKSGYAIINGKEVIESGPLNASWSAQACELFAVLR | | |
| | ALERLKGKVGTIYTDSKYAFGVVHTFGKIWENLADQEAKK | | |
| | AALTESRQKLKALFLPKRLSIIHCPGHQKGHSAEARGNRMA | | |
| | DQAARKAAITETPDTSTLL | | |
| 8351 | LTLEDEEKINPEVWHTEDEAGRLDIEPITIEIERPEDPIRIKQYP | 646 | N6.ZFERV |
| | ISPEGRRGLKPIIERLLKKGILEPCMSPHNTPILPVKKPDGSYR | | |
| | LVQDLREINKRTVTRYPVVPNPYTLLSKVSPEHQWFSVIDLK | | |
| | DAFWACPLAEESRDIFAFEWEDPETGRKQQLRWTRLPQGFT | | |
| | ESPNLFGQALEKLLQQFSPPEGVTILQYVDDLLIAGETEEEV | | |
| | REATIKLLNFLGEKGLKVSKSKLQFVEPEVKYLGHLISKGQR | | |
| | KLSPERVAGILSLPPPKSKREIRKLLGLLGYCRLWIEGYTETV | | |
| | KFLYEKLTEGGKIKWTEEDEKQLQELKQALTTAPVLSLPDL | | |
| | KKPFHLFVNTEEGIAYGVLTQEWGGCKKPVAYLSKLLDPVS | | |
| | RGWPTCLQAVAAVAILIEEARKLTFGGKLVVYTPHAVRAIL | | |
| | QQKAEKWLTDSRLLKYEAILLDKPRLELHVTKLVQNPAEFL | | |
| | YLEPKPVHHDCTETLEENTKRRPDLEDEELEEGEKWFVDGS | | |
| | SRVIEGKRKSGYAIINGKEVIESGPLNASWSAQACELFAVLR | | |
| | ALERLKGKVGTIYTDSKYAFGVVHTFGKIWENLADQEAKK | | |
| | AALTESRQKLKALFLPKRLSIIHCPGHQKGHSAEARGNRMA | | |
| | DQAARKAAITETPDTSTLL | | |
| 8352 | LTQEDEEKINPEVWHTEEEAGRLDIEPISIEIERPEDPIRIKQYP | 480 | N20.ZFERV |
| | ISLEGRRGLKPIIEQLLKKGILEPCMSPHNTPILPVKKPDGSYR | | |
| | LVQDLREINKRTVTRYPVVPNPYTLLSKVPPEHQWFSVIDLK | | |
| | DAFWACPLAEESRDIFAFEWEDPETRRKQQLRWTRLPQGFT | | |
| | ESPNLFGQALEKLLQQFSPPEGVQILQYVDDLLISGEDEEEV | | |
| | REATIKLLNFLGEKGLRVSKSKLQFVEPEVKYLGHLISKGSK | | |
| | RLSPERIAGILSLPPPKSKREIRKLLGLLGYCRLWIEKYTQTV | | |
| | KFLYEKLTEGDKIKWTEEDEKQLKKLKQKLTSAPVLSLPDL | | |
| | KKPFHLFVNTEKGVAYGVLTQEWGGVKKPVAYLSKLLDPV | | |
| | SRGWPTCLQAIAATAILIEEAQKLTFGGKLIVYTPHNVRTILN | | |
| | QKAEKWLTDSRLLKYEAALMNKPRLELHVTKIVEDPAEFT | | |
| | YTPVQHDCTLTLKRQKK | | |
| 8353 | LTLEDEEKINPKVWHTGREAGRLDIEPISIEIERPEDPIRIKQY | 465 | N14.ZFERV |
| | PISLEGRRGLKPIIEDLIKKGILEPCMSRHNTPILAIKKTDGSY | | |
| | RLVQDLRAINERTKTRFPVVANPYTLLNRVSPEDTWYSVID | | |
| | LKDAFWTCPLAEGSRDYFAFQWEDPDTNRKQQLRWASLPQ | | |
| | GFVDSPNLFGQALEQLLSQFSPGEGTKILQYVDDLLVAGETE | | |
| | EDVRECTIELLNFLGEKGLKVSKSKLQFTEPEVKYLGHWITK | | |
| | GKKKLDPERVAGILELPPPKNKRQVRQLLGLLGYCRQWIEG | | |
| | YSEKVKFLYEKLTTDKIKWTEQDEKELQRLKEALITAPVLSL | | |
| | PDVKKKFQLFVDVSNHTAHGVLTQEWAGDKKPVGYLSKLL | | |
| | DPVSRGWPTCLQAIVAVALLIEEAKKITFGGDLVVYTPHNV | | |
| | RLILQQKAERWLTDARLLKYEAILIHAPELELRVTKASNPAE | | |
| | FLYLGK | | |
| 8354 | LPTQVEDAVVPWVWSTEGPGKSIAVEPVVIELKEGEQPVRI | 647 | N55.ZFERV |
| | KQYPMKPEARRGIKPIIEQFLKLGILEECQSEYNTPILPVKKP | | |
| | NGEYRLVQDLRAVNKITEDIYPVVANPYTLLTSLSPEHQWF | | |
| | TVIDLKDAFFCIPLEPESQKIFAFEWENPETGRKTQLTWTRLP | | |
| | QGFKNSPTIFGEQLAKDLEEWKAPPESGVLLQYVDDLLIATE | | |
| | TKEACIKATIALLNFLGQKGYRVSKKKAQLVQQEVIYLGYEI | | |
| | SGGQRKLGPDRKEAICQIPKPKTVKELRSFLGMVGWCRLWI | | |
| | PNYGLLAKPLYELLKEGSDKLNWTKEAEKAFQELKQALTT | | |
| | APALGLPDLSKPFQLFVNEKQGIALGVLTQKLGPWRRPVAY | | |
| | LSKQLDTVAAGWPSCLRAVAAVAILIQEARKLTMGQKMVV | | |
| | YVPHAVSAVLEQKAGHWLSSSRMLKYQAILLEQDDVELAV | | |
| | TNVLNPATFLYSEPEPVHHDCLETIEASYSSRPDLKDTPLED | | |
| | AEEWFTDGSSYVISGKRKSGYAVTTCKEVIESGPLNPSYSAQ | | |
| | KAEIIALTRALELAKGRTVNIYTDSRYAFGVVHAHGAIWKE | | |
| | LADREAKKAAKTELQQSLKALFLPKRLSIIHCPGHQKGHSA | | |
| | EARGNRMADQAARKAAITETPDTSTLL | | |
| 8355 | LPKQVEDAVVPWVWSTEGPGKAVAVEPVVIELKPGEQPVRI | 647 | N54.ZFERV |
| | KQYPMKREARKGIKPIIERFLKLGILEECQSPYNTPILPVKKP | | |
| | NGEYRLVQDLRAVNKITVTIYPVVPNPYTLLSSLSPEHQWFT | | |
| | VIDLKDAFFCIPLEPESQKIFAFEWEDPETGRKTQLTWTRLPQ | | |
| | GFKNSPTIFGEALAKDLQEWKAPPESGTLLQYVDDLLIATET | | |
| | KETCIKATIALLNFLGEKGYRVSKKKAQLVQQEVTYLGYEIS | | |
| | KGQRRLSPDRKEAICQIPKPKTVRELRSFLGMVGWCRLWIP | | |
| | NYGLLAKPLYELLKEGSDPLNWTEEEEKAFQQLKQALTTAP | | |
| | ALGLPDLSKPFQLFVNEKQGIALGVLTQKLGPWKRPVAYLS | | |
| | KQLDPVAAGWPSCLRAVAATAILIQEARKLTLGQKMVVYV | | |
| | PHTVSAVLEQKAGHWLSSSRLLKYQAILLDQPDVELKVTKV | | |
| | INPATFLYSEPEPVHHDCLETIEASYSSRPDLKDTPLEDAEEW | | |
| | FTDGSSYVINGKRKSGYAIITCKEVIESGPLNPSYSAQKAELI | | |
| | ALTRALELAKGRTGNIYTDSKYAFGVVHAHGAIWKELADR | | |
| | EAKKAAKTDLQQPLKALFLPKRLSIIHCPGHQKGHSAEARG | | |
| | NRMADQAARKAAITETPDTSTLL | | |
| 8356 | LPTQVVTAVVPQVWLSPHLDLFYRTDFPKAWAETEGPGKAI | 666 | N38.ZFERV |
| | QVEPVVIELKPGEQPVRIKQYPMSPEARRGIKPIIERLLKLGIL | | |
| | EPCQSPYNTPILPVKKPDNGEYRLVQDLRAVNKRTVTIYPV | | |
| | VPNPYTLLSSLSPEHQWFTVIDLKDAFFCIPLAPESQPIFAFE | | |
| | WEDPETGRKTQLTWTRLPQGFKNSPTIFGEALAKDLQEFPA | | |
| | PPEGVTLLQYVDDLLIAAETEEACLKATIALLNFLGQKGYR | | |
| | VSKKKAQLCQQEVTYLGYEISGGQRKLSPDRKEAILQIPKPK | | |
| | TVKELRSFLGMVGYCRLWIPGYAELAKPLYELLKEGSDKLN | | |
| | WTEEAEKAFQELKQALTTAPALGLPDLSKPFQLFVNEKQGI | | |
| | ALGVLTQKLGPWRRPVAYLSKKLDPVAAGWPSCLRAVAA | | |
| | VAILIQEARKLTMGQKMVVYTPHAVSAVLEQKADRWLSNS | | |
| | RMLKYQAILLDKPRVELHVTKVLNPATFLYSEPEPVHHDCL | | |
| | ETLEESYSRRPDLKDTPLEDAEEWFTDGSSYVISGKRKSGYA | | |
| | IITCKEVIESGPLNPSYSAQKAELIALTRALELAKGRTVNIYT | | |
| | DSKYAFGVVHAHGAIWKELADREAKKAAKTELQQSLKALF | | |
| | LPKRLSIIHCPGHQKGHSAEARGNRMADQAARKAAITETPD | | |
| | TSTLL | | |
| 8357 | LTGQVVTAVVPQLWLTEDEGGLIQVEPVTIELKPGEQPVRIK | 651 | N37.ZFERV |
| | QYPLSPEARRGIKPIIERLLKKGILEPCQSPYNTPILPVKKPDN | | |
| | GTYRLVQDLRAINKRTVTIYPVVPNPYTLLSSLSPEHQWFTV | | |
| | IDLKDAFFSVPLAPESQPIFAFEWEDPETGRKQQLTWTRLPQ | | |
| | GFKNSPTIFGQALEKTLQEFPAPPEGVTLLQYVDDLLIAAET | | |
| | EEECLKATIALLNFLGQKGFKVSKKKLQLCQPEVTYLGHEIS | | |
| | GGQRKLSPDRVAAILQLPKPKTVKELRSFLGLVGYCRLWIP | | |
| | GYTELAKPLYELLKEGSPPKDKLNWTEEAEKQFQELKQALT | | |
| | TAPALGLPDLKKPFHLFVNEKEGIALGVLTQKLGGHRRPVA | | |
| | YLSKKLDPVAAGWPSCLRAVAAVAILIQEARKLTMGQKMV | | |
| | VYTPHAVSAVLEQKADRWLSNSRMLKYQAILLDKPRVELH | | |
| | VTKVQNPATFLYSEPEPVHHDCLETLEESYSRRPDLKDTPLE | | |
| | DAEEWFTDGSSYVISGKRKSGYAIITCKEVIESGPLNPSYSAQ | | |
| | KAELIALTRALELAKGRTVNIYTDSKYAFGVVHAHGAIWKE | | |
| | LADREAKKAAKTELQQSLKALFLPKRLSIIHCPGHQKGHSA | | |
| | EARGNRMADQAARKAAITETPDTSTLL | | |
| 8358 | IPEEVEQAVVPWVWETDTPGKSKAAQPVVVELKEGKEPVRI | 632 | N56.ZFERV |
| | KQYPIKPEARQGIKPIIDKFLKLGILEECESEYNTPIFPVKKPN | | |
| | GEYRLVQDLRAINEITKDIYPVVANPYTLLTSVSEKHEWFTV | | |
| | IDLKDAFFCIPLEKESRKLFAFEWENPDTGRKTQLTWTRLPQ | | |
| | GFKNSPTIFGNQLAKELEEWKTTQVKVPPESYVLLQYVDDI | | |
| | LIATEEKETCIKLTISLLNFLGQGGYRVSKKKAQLVRQEVIY | | |
| | LGCEISQGQRKLGTNRIEAICAIPEPRNHQELRSFLGMVGWC | | |
| | RLWILNYGLIAKPLYEALKEPRLTWGKQQEKAFLELKQALT | | |
| | EAPALGLPDLSKDFQLFVNERQRLALGVLTQRLGPWKRPVG | | |
| | YFSKQLDTVSAGWPSCLRAVAATVILIQEARKLTLGRKIEVY | | |
| | VPHMVTAVLEQKGGHWLSSSRMLKYQAILMEQDDVELKIT | | |
| | NLINPAEFLSEEGPLAHDCVEIIEQTYASREDLKDVLLEQAEE | | |
| | WFTDGSSFGKNATGWAVSNNPQYSAQKAEIIAYIRAKGRTG | | |
| | NFYTDSRYAFGVVHAHGAIWKELADREAKKAAKTELQQSL | | |
| | KALFLPKRLSIIHCPGHQKGHSAEARGNRMADQAARKAAIT | | |
| | ETPDTSTLL | | |
| 8359 | LTGQVVTAVVPQLWLSLPPDPHLDLFYRTDFPKAAAIDDSL | 447 | N67.ZFERV |
| | WSESEDEGGLIQVEPVKIKLKPGERPVRIKQYPLSPEAEQGIK | | |
| | PIIERLLKKGILEPTQSPYNTPILPVKKPDNGTYRLVQDLRAI | | |
| | NKLTVTITPVVPNPYTLLSSLSPKHQWFTVIDLADAFFSVPL | | |
| | DPESQPIFAFTFENQQYTWTRLPQGFKNSPTIFSQALKKTLQE | | |
| | FPALPEGVTLLQYVDDLLIAAETEEECLKATIALLNFLAQKG | | |
| | FKVSKKKLQLCQPEVTYLGHEISGGQRKLSPDRVAAILQLPK | | |
| | PKTVKELQSFLGLVGYCRTWIPDYTELAKPLRELLRHEGSPP | | |
| | KDKLNWTEEAEKQFQELKKALTTAPALGLPDYKKPFHLHV | | |
| | NEKEGIALGVLLQKHGGHRRPVAYLSKKLDPVAAGWPSCL | | |
| | RAVAAVAIAIQEARHLVMGHKMVVYTPHA | | |

TABLE 13
Exemplary RT domains derived from a Cas-RT

| SEQ | | |
|---|---|---|
| ID | | |
| NO: | RT sequence | name |
| 8199 | STIDVTLKEVADPIRLKLAWTKIKKKGSIGGVDGVTISSFNANLE | A0A0M9DZB1 |
| | VNLSELSNQILTNQYTPEPLQAAHIPKPGKSEKRQLGLPSLKDKI | |
| | VQSSLASILSDFYEIHFSNCSYAYRPGKGSVKAIGRVRDFLNRKN | |
| | YWIASVDIDNFFDSVDHEICTSILKEQISDQSIIRLISLYFSSGM | |
| | IKFDQWQDTEIGIPQGGAISPVISNIYLNKLDHFLHTLNAFFVRY | |
| | ADDIILFSNTQQSLSETYQKTNEFLNKKLNLKLNALDNPIINVSK | |
| | GFSFLGIYFHRCQLKIDFKRIDEKIEKMKYIIHKQKQIDAVIKEI | |
| | NEFFNGVQRHYGNIIPDSYQLKNLESTVLDELSIFLAKMKNEGHI | |
| | NSKKACKLVLDPLVFMSERTKSQRDAVIDKIIADAFTIVDQKKDT | |
| | DEKRIEKSVDSAIHQKRQAYAKKIATETE | |
| 8199 | STIDVTLKEVADPIRLKLAWTKIKKKGSIGGVDGVTISSFNANLE | A0A0M9DZB1 |
| | VNLSELSNQILTNQYTPEPLQAAHIPKPGKSEKRQLGLPSLKDKI | |
| | VQSSLASILSDFYEIHFSNCSYAYRPGKGSVKAIGRVRDFLNRKN | |
| | YWIASVDIDNFFDSVDHEICTSILKEQISDQSIIRLISLYFSSGM | |
| | IKFDQWQDTEIGIPQGGAISPVISNIYLNKLDHFLHTLNAFFVRY | |
| | ADDIILFSNTQQSLSETYQKTNEFLNKKLNLKLNALDNPIINVSK | |
| | GFSFLGIYFHRCQLKIDFKRIDEKJEKMKYIIHKQKQIDAVIKEI | |
| | NEFFNGVQRHYGNIIPDSYQLKNLESTVLDELSIFLAKMKNEGHI | |
| | NSKKACKLVLDPLVFMSERTKSQRDAVIDKIIADAFTIVDQKKDT | |
| | DEKRIEKSVDSAIHQKRQAYAKKIATETE | |
| 8360 | GQYQLQDAYGYCSYPRPQAAKSLLEKSLSDASLHQACQTMYPRQA | F2K1V9 |
| | NFDSSDTDEEHHDAIDELLTKLYVSRERIFKREFTPSQLHSVEIE | |
| | KPEGGTRLLSVPNWHDRTLQKAVTECLGNTLEHIWMKHSYGYRKG | |
| | HSRLQARDQINQYIQQGYEWVLESDIESFFDSVNWLNLEQRLKLL | |
| | LPNEPLVPLLMQWVSAAKQTEDEQTLARHNGLPQGAPISPILANL | |
| | LLDDLDQDMIAKGHQIVRYADDFVLLFKSKAAAESALDDIITALK | |
| | EHHLAINLEKTRIVEASQGFRYLGYLFGGSQYKLILNGKTLKGET | |
| | TTEAVDAATAEKVFKQYANDNGVDGEWTYDDATKTFTVTEGGSKL | |
| 8361 | GWLYNQMAMPETIFQAWYKVASNDGRPGWDNKSIEDYSLQLEENL | B3EIR7 |
| | KALSQALLTGTYKQGPLMKLVLLKPDGKDRVLLIPGVMDRVAQTA | |
| | AAIVLSPIIEAELGNCTFAYRPGISREGAAREIDRLHREGYQWVL | |
| | DADIRSFFDNVRHDLLFQRLVELIDDKEMISLLHRWLTAEIVDGI | |
| | NPRIQNTMGLPQGCPISPALANLYLDRFDETMEKEGFKLVRFADD | |
| | YLVLCKTRPKAEAALKLSETALAELKLELHSDKTRITTFAEGFKY | |
| | LGYLFIRALVIPTKMHPEEWYDKLGKFKLRKKSEHALPSDPDAMT | |
| | GETAKFELETDQGEKIELTKNELLQTEFGCKLLESLDKKQLSVDE | |
| | FLEKVARQDEERQKEKRDALKKLYSPFLNT | |
| 8220 | GTANELPLLEQALSDDRLLAGWERVRANAGGPGVDGVTVEQFGGK | A0A3N4U3Z7_9 |
| | VLRALAGLRQRVTASHYQALPLRRIEITRPGKAPRVLAVPCVADR | BURK |
| | VVQSAVALTISPRLDPGFEDFSFGYRPGRSVPRAVQHLAEARDSG | |
| | LVWVAEADIQSCFDRIPWAALLQRLGEVLPDAGLLALIQHWLSLP | |
| | LQWPDGHQQVRCMGVPQGSPLSPLLSNVFLDGMDKELAAGPWRVI | |
| | RYADDFVIAAASREEARRGLAQAARWLRRLGLRLNLDKTRVIHFD | |
| | QGFSFLGVRFRGRQMSAVQPGAEPWVLPRATQPRPHSPSSKPAQH | |
| | SRSPAPTARASAPATPPSAQPEPLGPAAPSPNAAASAQPSQPRAA | |
| | DATLQDLQRLSVAPPNEPSPPRLRT | |
| 8221 | STLPTPSSTDQDSPPPFWTLARLAEALEHVSARQGGAGADEQTLA | A0A080MH79_ |
| | EFAADAEAQLGLLALQLTQGSYRPAPARLIPVAKPGGGVRELLLP | 9PROT |
| | AVRDRIVQSALARYLADLLEPDFGEASHAYRPGHSVATALHRLQA | |
| | LRDGGLVFVAVCDIHHFFDSVDHRRLFSLLDDLPLERRLREQMKT | |
| | CVRIEVADVQGQGAWSLARGLAQGSPLSPVLANLFLMAFDAACAR | |
| | AGLALVRYADDCVLACASETEAQSALAFAADALENIGLALNTRKS | |
| | RLASFAEGFEFLGAFCGAEGMLGGRPGEAACLPPTTGPVHEAAAA | |
| | DDERPPSHGHRPRLR | |
| 8221 | STLPTPSSTDQDSPPPFWTLARLAEALEHVSARQGGAGADEQTLA | A0A080MH79 |
| | EFAADAEAQLGLLALQLTQGSYRPAPARLIPVAKPGGGVRELLLP | 9PROT |
| | AVRDRIVQSALARYLADLLEPDFGEASHAYRPGHSVATALHRLQA | |
| | LRDGGLVFVAVCDIHHFFDSVDHRRLFSLLDDLPLERRLREQMKT | |
| | CVRIEVADVQGQGAWSLARGLAQGSPLSPVLANLFLMAFDAACAR | |
| | AGLALVRYADDCVLACASETEAQSALAFAADALENIGLALNTRKS | |
| | RLASFAEGFEFLGAFCGAEGMLGGRPGEAACLPPTTGPVHEAAAA | |
| | DDERPPSHGHRPRLR | |
| 8362 | LYVEQLATAWHLVQRGSRAAGIDGITVDLFTGIAREQIHQLYRQM | A0A1Z3HGW6 |
| | RQERYVARPAKGFYLAKQKGGHRLIGIPTVRDRIVQRYLLQSIYP | |
| | SLENAFSDAVFAYRPGLSIYAAVKRVMERYRYQPTWVIKADIQQF | |
| | FDQLSWPLLLHQLDQLSLPATWVQWIEQQLKAGIVVSGQFYQPGQ | |
| | GVLPGSILSGALANLYLNDFDRHCLEADIPLVRYGDDCVAVCQSY | |
| | LEASRSLALMQDWIEGLSLSFHPEKTTIIPPGQAFVFLGHRFRNG | |
| | TVEGPARQKAEGRRQKAPQPGYGPPQLCSIVKSPRRMLATSTDDY | |
| | WRDGMTTKL | |
| 8363 | FTQEHLHFAWLQVCAGSKTAGVDGISVELFESMATEQLQNLVYQL | K9QDL5_9NOS |
| | NNETYTASPAKGFYIPKKNGDKRLVGIPTVRDRIIQRLLLDELYF | O |
| | PLEGTFLDCSYAYRPGHNILQAVQHLYGYYQYQPKWIIKADVADF | |
| | FDNLSWALLLTFLEKLSLEPSVLQLIEQQLQSGMIIAGQYRNFGK | |
| | GVLQGGILSGALANLYLTNFDKKCLSQGINLVRYGDDFVIACNSW | |
| | QEANRILDKITVWLGEVYLTLQSEKTQIFTPNDEFTFLGYRFAGG | |
| | EVYAPPPPKPVLKGEWVINDSGNPYFRTKPRPKKPVSHPPKACSI | |
| | DKPINFPRASLSHYWQETMTT | |
| 8249 | ETSVRHLGELTYPLRASAAFQRQALTGEPDLLTEIAAPDSLLNAW | A0A1Q5PUX9 |
| | RYVFTRDAKDGYLLQQSQQIAADPDRFVAALSGALLSGRYQPEPQ | |
| | VEVLIPKKGKTSAMRELSIPSIRDRVVERAVLNAIIDRADLLQCS | |
| | ASFAFRRGLGVQAATHEITQLRDSGNRYVLLTDIANYFGRINIAD | |
| | SLRVLQRGLFCSRTLALLRFIAKPRRVVGRRRIRSRGLAQGSCLS | |
| | PLLANLALTDIDFALADTGVGYVRFADDILLCAPSRTELAASQRL | |
| | LASLAAHQGLQLNEEKTMHTSFDAGFCYLGVDFTAHQPVTDLHYG | |
| | VKHTKQPAKV | |
| 8249 | ETSVRHLGELTYPLRASAAFQRQALTGEPDLLTEIAAPDSLLNAW | A0A1Q5PUX9 |
| | RYVFTRDAKDGYLLQQSQQIAADPDRFVAALSGALLSGRYQPEPQ | |
| | VEVLIPKKGKTSAMRELSIPSIRDRVVERAVLNAIIDRADLLQCS | |
| | ASFAFRRGLGVQAATHEITQLRDSGNRYVLLTDIANYFGRINIAD | |
| | SLRVLQRGLFCSRTLALLRFIAKPRRVVGRRRIRSRGLAQGSCLS | |
| | PLLANLALTDIDFALADTGVGYVRFADDILLCAPSRTELAASQRL | |
| | LASLAAHQGLQLNEEKTMHTSFDAGFCYLGVDFTAHQPVTDLHYG | |
| | VKHTKQPAKV | |
| 8364 | IETRREVEAFEANSQSNLKRIADQLLHRKFIFPAAKGVPIQKAKG | >BIMetSil55537 |
| | KRGDIRPLVVAKVEARIVQRAIHDVLIEVPSIRRYVRTPYSFGGV | 2322\| |
| | RKEKDDSVSAVPAAIDAAMAAIGDGFSYYIRSDITAFFTKIPKSA | [Methylocella |
| | VAALVSDAVGHQSEFMDLFRRAIHVELENMARLARTVNAFPIYDI | silvestrisBL2] |
| | GVAQGNSLSPLLGNILLYDFDQQMNGNPDAVCLRYIDDFIIFAKT | |
| | QQLAENMFQKAIHILASHGMSVAKHKTVKGLVRDKFEFLGIEF | |
| 8365 | PTIRTEIEQFSLSLEKNLRRIADQLREKRYVFSQSYGVAVKKKNN | >DS_gi\|8270206 |
| | PSKKRPIVISPIPNRIVQRALLDVVQEIPSVRAKLDSGFNFGGIA | 3\|ref\| |
| | EIGVPQAILKAYKTALEKPYFIRTDICAFFDNIPRSQALEIITSA | YP_411629.1 |
| | SKDDDFNTLLTQATTTELSNLITLGRDKELFPLEGKGVAQGSCLS | [Nitrosospira |
| | PVLCNLLLDDFDKKMNARGIVCIRYIDDFILFAPSESKAFKAFAS | multiformis |
| | ASAFLEKLNLSVYDPRHSPDKAEHGVSNKGFEFLGCSV | ATCC25196] |
| 8366 | SSDEIKRDAEEFESRLPDSLVEIQRSLSKQTFTFLQQTGVAQKKP | >DS_gi\|7173551 |
| | GGKARPLVLAPIPNRVVQRALLDVLQRRVRFVRKVLDTPTSYGGI | 5\|ref\|YP_277063.1 |
| | PTKRVAMAISDARDAMRNGARFHIRSDIPAFFTKINKDRVQDLLR | [Pseudomonas |
| | SHINCDATLKLLDLAITTDLANIDDLRRQGLNEIFPIGIEGVAQG | syringae |
| | SPLSPLLANIYLADFDVAMNADGITCLRYIDDFLLLGESLSNVDR | pv |
| | AFNRALKTLDKIGLSAYDPRVDKVKASRGSTDKGFDFLGCNV | phaseolicola |
| | | 1448A] |
| 8367 | GIDGVMLSQLKEYLILNWQDIENQLILGTYEPNIVQVYELLSKKG | >PF_WP_05159 |
| | KVREIYKFTIIDNFIQKAVSLILQDKLDCLLSDNNYSFRKGKGTI | 2781.1 |
| | DVIRKGLELIEEGYEYIVEIDIKKYFENIDHVLLSKMLFDIIDDK | [Clostridium |
| | VLISLIMKYQNCLIQKDGKIKRKNKGLITGSSISPVISNLYLMDL | saccharogumia] |
| | DRQYLEYNYIRYCDNIYIFINNKDDGLTLINDISKCLKDKYKLEI | |
| | NQNKTSITHYLSKRMLGYYF | |
| 8368 | GIDGLYLSELRDDWNINGERYLSLLRKGKYKPGIVQIYEIVNYTG | >PF_WP_00875 |
| | KRRSISSFNSIDRLVLRCLATSLEKYYDSIFSSSSFAFRPGLGVD | 1399.1 |
| | KAVATFANNLNTGLTRVAIIDIKHYFDSIPIDRLEMILKRIIDDN | [Lachnoanaerobaculum |
| | VLLSLFHNLLYCRISEENVIKTKSKGILQGSPISPFLGNLYLSLL | saburreum] |
| | DTQLESMHVSFCRYCDDIAMFFASFEEAKETYTKVYDILKNDLEM | |
| | DINPQKSGIYEGIKQNYLGYSF | |
| 8369 | EIDGLHLSELRDYWDINGERYLSMLRAGKYKPGIVQIYEIINYTG | >PF_WP_060932241.1 |
| | KRRSISSFNSVDRLILRCLATSLEKYYNSIFSACSFAFRPGLGVD | [Lachnoanaerobaculum |
| | KAVAAFVSNLNKGLDKVVVIDIKHYFDSIPIDRLEMILKRIIDDK | saburreum] |
| | ILLSLFHKFLYCRISEENIIKTKNKGILQGSPISPFLGNLYLSLL | |
| | DTQLESMQIQFCRYCDDITMFFSSFDEAKEAYTKVSNILKNDLEM | |
| | DIHTQKSGIYEGIKQNYLGYNF | |
| 8370 | GIDGIFVKDFEEYWILNGQKILKQVMNGVYMVSPVQLREIIMPTG | >PF_CVI70780.1 |
| | KHRIIAHYTCTDRLITRILAESLQKEVDDSLSEYSYAYRKQRGVI | [Eubacteriaceae |
| | KAVEQAAAYMQAGKIWVLELDIENYFNNINLTLMEEKIREIILDK | bacterium |
| | NLFSLMEQYLRCEVMEEEYTKTYIKDKGLVQGCSLSPVLSNIYLN | CHKCI004] |
| | KLDQQMEKEGLSFCRFGDNINIYFYNKLEAAEWYAKIKAIIENEF | |
| | DLHLNIRKSGIYLGVNRIFLGYSF | |
| 8371 | GLDGVKLSELRAYWETNGKKIKESIFNGTYKVGAVEQRQIVNRKG | >PF_CRL43259.1 |
| | KKRTISLMNSIDRFIFRALYQKMASEWEKQFSQYSYAYQNNKGVL | [Roseburia |
| | TAVEQAAKYMEEGKDWSVELDIQNFFDNINHSIIISKLKAGIEDV | inulinivorans] |
| | RVLDLLIAYLTCTLLDDHAFHQMEQGVLQGGPLSPLLANVYMNEL | |
| | DHYMEKQGYSFGRFGDDINIYCSTYEEATVAFSDVTARMEKIEQL | |
| | PLNHGKTGIFKGINRKYLGYRF | |
| 8372 | GPDTITTDDLKKAGDQFLDKLKNNIVNGNYKQGKTKQYRIPKNDD | >PF_KJR40057. |
| | TFRYIYVLNTTDRLVHKTIADYISPIVDNIISNSAYAYRRGLNTK | 1 [Candidatus |
| | GAANALNNALKEGYTSGIKADISEFFDSINISALSMMIDSLFPFE | Magnetoovum |
| | PLADFINGILENNTRDGIKGILQGSPLSPLLSNLYLTRFDSDMES | chiemensis] |
| | KGFFKLIRYADDFVLLLKTASSYEETIKHVEDSLSTLGLKLKPEK | |
| | TTEITQGKAINFLGYVI | |
| 8373 | GLDGVSVQSFGDQAASHLEDLRQALQAGNYTPEPHQRIKVPKLDG | >PF_WP_01370 |
| | SGELRPLSLPTIKDKIVQEAVRRIIEPLFEPEFLPCSYAYRPGQG | 7702.1 |
| | PRRAIGRAIHYLEHDKCRWAVHADFDKFFDTLDHEVLLRRLQEKI | [Desulfobacca |
| | KELPVLKLVRMWLRTGSIGAKGTYDDADLGVGQGGILSPLLSNIY | acetoxidans] |
| | VHPLDVYLTDKGHRYIRYADNVLILADSQPRGTEGLNDLIYFSQE | |
| | MLKLRLNPEAKPLRHVADGFTFLGIHF | |
| 8374 | GLDGVEIDDQHTDADKMVSALIKELRTGAYVPVPYARGAIPKFDE | >PF_KKO17867.1 |
| | QNQWRKISLPSVRDKVVQQAFVEALGPVFNKTFLDCSYAYREGKG | [Candidatus |
| | PVKAIKRVEHILHTHHIRWVTTMDIDNFFDTMDHDIFIGEFTKKV | Brocadiafulgida] |
| | AEPEILQLVRLWLKAGCISARGDWIEPYDGIAQGAVVSPLFSNIY | |
| | LHPLDCFAIGNNCLYVRYSDNLIVLSETKETLYLWYEQLKSFLED | |
| | RLRLRLNEDPYPFKDKERGFVFLGIFF | |
| 8375 | GLDNVTVESFGNRLDQHISKLQKEIMEHRYVPKPLKSIHVPKYNK | >PF_KHE91657.1 |
| | ENEWRGLALPSVSDKVVQAALLQVVEPLGEKLFMDSSYAYRKGKG | [Candidatus |
| | HYKAIRRVEHCLGNRKKSWVVHRDIDNFFDTLNYDRLIDQFSALV | Scalinduabrodae] |
| | DGEPVMTELVALWCRTGLVEAGGRWRGVQSGIRQGNIISPLLSNL | |
| | YLHPLDEFAARLRIDWVRYCDDYLILCDSRKDAISADRLIKEYLK | |
| | EPLCLKLNNSGLSPCHIDEGFTFLGVSF | |
| 8376 | GLDGITVEEFGHRLDQHITKLQKDIRERRYIPQPAAVTYIPKFNE | >PF_WP_007220853.1 |
| | ENEWRELGLPSVADKVVQAAMLEVVEPLAEKMFLDCSYAYRPGTG | [Candidatus |
| | HYKAIRRVENSLNNRKKTWVVQRDIDNFFDTVDHNRLMEQFSALV | Jetteniacaeni] |
| | QGEPTMVELVALWCRMGLVEKNGRWRNVQAGIRQGGVISPLLANL | |
| | YLHPLDVFATKLGVDWIRYADDYVILGESQEEVVSSDVQIVEFLK | |
| | DSLGLMLNRDESSPKHIDEGFTFLGVRF | |
| 8377 | GVDGVTISSFNANLEVNLSELSNQILTNQYTPEPLQAAHIPKPGK | >PF_KPA10619.1 |
| | SEKRQLGLPSLKDKIVQSSLASILSDFYEIHFSNCSYAYRPGKGS | [Candidatus |
| | VKAIGRVRDFLNRKNYWIASVDIDNFFDSVDHEICTSILKEQISD | Magnetomorums |
| | QSIIRLISLYFSSGMIKFDQWQDTEIGIPQGGAISPVISNIYLNK | p.HK1] |
| | LDHFLHTLNAFFVRYADDIILFSNTQQSLSETYQKTNEFLNKKLN | |
| | LKLNALDNPIINVSKGFSFLGIYF | |
| 8378 | GIDGVSISEFETARDKNLQELSQQILYSQYTPEPLQAVQIPKPGK | >PF_ETR69258.1 |
| | TEKRQLGLPSLKDKIVQSSLASLLSDFYDPLFSNCSYAYRPQKGS | [Candidatus |
| | VRAIGRVKDFLNRKNHWAAPVDIDNFFDTVNHETCISILQDKISD | Magnetoglobus |
| | IDIIRLIRLYFSSGKIQFDKWQDTIIGIPQGGALSPVLSNVYLNE | multicellularis |
| | LDQYLHAIQANFVRYADDIILFANTRQFLLDFYEKTRHFLESKLQ | str. Araruama] |
| | LKLNQTSHPVMSMEKGFAFLGIYF | |
| 8379 | RVSLKREIHLPEKEIENLFRALQNSTYIPEPPQKIELKKHDKIRP | >PF_WP_025270209.1 |
| | ITIASKKDKIVQALLHEYLTELFDSSFSDKSYAYRPNKGPLKAVN | [Hippea |
| | RTFDYIKRGEKYVLKTDIKDFFETIDHSLLICMLKEKIKDDSLID | sp. KM1] |
| | LIMMYIKIGTVKNLEYEDHNLGVHQGNIISPILSNIYLDRMDKFL | |
| | ERHGFNFVRFADDFVVFAKTHDRIELIHRNLKRFLKVYKLGLNEE | |
| | KTYITTTDSGFAFLGAYF | |
| 8380 | GLDNISYIEFKQNFTSQIKELIETILKGTYSPEPLKKIEIQKEDS | >PF_WP_04699 |
| | LEKRPIALSSIKDKLVQRVLYKALNDYFDETFSNKSYAYRKDKST | 6094.1 |
| | LNAINRVGQFIQEQNHFILKTDIDNFFESINHDKLLTILDKHIQD | [Arcobacter |
| | KSIIRLISLFLQIGSFKEFDYFEHEDGVHQGDILSPLLSNIYLDL | butzleri] |
| | MDKWLEKYDIFFVRYADDFVVFSKKEDELKTIKENLEKFLESLDL | |
| | KFGIDKTYFTTIQKGFSFLGVYF | |
| 8381 | GIDNLSELNEHFIHKLKQSCLNQTYVPEPVLQKLIPKSDGENYRK | >PF_CZE46369.1 |
| | LAISSLKDKLIQKVLANELAWYFDKHFSDKSYAYRPGKSYKNAIF | [Campylobacter |
| | RLRDFLRVKPYFVIKSDIKDCFESINHSKLVALLAKYIKDKRVLN | geochelonis] |
| | LVEIWIKNGIFNRQTYIKHSKFGIHQGDVLSPLLANIYLNQMDKF | |
| | LETNNEIFIRYADDFVILADDEKFVQAKINSLKTFLSTIDLSLKD | |
| | TKTAIYSPTQSFEFLGVSF | |
| 8382 | GLDELSMDELCTEAFFAELKDEILNLSYSPQPLKRAFIPKENKDE | >PF_WP_021087740.1 |
| | FRKLAIPSLKDKFTQNILIGELSSYFDKGFSNRSYAYRSGKSYSN | [Campylo |
| | AIFRARDFCLTHDFVLKTDIKDFFENINHEKLLEILRSNIKDTRI | bacterconcisus] |
| | IRLIELWIKNGIFEHFDYTSHTKGVHQGDVLSPLLSNIYLDQMDK | |
| | FLEHSSIEFVRYADDFVLFFGSREACEQALAGLKDFLVTINLSLN | |
| | EAKTSLHDKDSEFTFLGVNF | |
| 8383 | GFDGLSADDICSGEFYAELKSEIFSLSYSPQPLKRAFIPKEAKDE | >PF_WP_005873073.1 |
| | LRKLAVPSLKDKFVQNILTRELSGYFDKSFSNRSYAYRNGKSYAN | [Campylo |
| | AIYRARDFFQIFSFAVKTDIKDFFENIDHEKLLEILRANIRDARI | bactergracilis] |
| | IRLIELWIKNGIFERFDYRAHTKGVHQGDVLSPLLSNIYLNQMDK | |
| | FLENSGVEFVRYADDFVMFFASYEAAEMRLARLKDFLKTISLSLN | |
| | EAKTSIHGKDSEFVFLGVSF | |
| 8384 | GQTIDAFRRDRDRNVTRISDSLKNGTYAPSPLRGVKISKNGGGFR | >W_[Fretibacterium |
| | RLGVPTVKDRIVFQGANRLLADVWDPLFAPLSFAYRSGRSIADAI | fastidiosum] |
| | DAVIERIRKGRVWFVKGDIKGCFDELSWDVLSACLHDWLPDESLR | 479198758 |
| | RLVNQAIRVPVVEGGQIRPRLRGIPQGSPLSPLLANLYLHSFDLQ | |
| | MLQQGFPVIRYADDWLLLVGSEPEAQAALQTAQGILSVLNIAINE | |
| | EKSGIGNLRCESVAFLGHRI | |
| 8385 | LCQVFGVHRSSYRYWKNRPEKPDGRRAVLRSQVLELHGISHGSAG | >DS_fid\|186781 |
| | ARSIATMATRRGYQMGRWLAGRLMKELGLVSCQQPTHRYKRGGHE | 73\|locus\|VBIShi |
| | HVAMPKWVILYCRRWMEAPMQSCENGELITRTRGTPQGGVISPLL | Boy33460_0060\| |
| | ANLFLHYAFDLWMEREYRGVPFERYADDIVVHCSRMSDATRLKNR | [Shigellaboydii |
| | LSERFSEVGLVLNAGKTNIAYIDTFKRRNVATSFTFLGYDF | Sb227] |
| 8386 | GVDGFTVAHFEKKLTDNLTELHHELVTGTWNPEPYLRVEIAKNET | >PF_WP_00748 |
| | EKRKLGLLCIKDKIVQQAIKTAIEPQMDKTFLNISYGYRPGKGAE | 1073.1 |
| | RAIRRTIQELKKLKNGYIAKLDIDNYFDNINQERLFTRLGNWLKD | [Bacteroide |
| | DETLRLIKLCVQTGIVNPQLKWERTTKGIPQGAILSPLLANFYLH | ssalyersiae] |
| | PFDQFAISKAPMYIRYADDFLIASPSEKQTKEAVELIKEELADTF | |
| | YLQLNKPLVCNFHDGVEFLGIIV | |
| 8387 | GIDGFTLSHFEKRLNDNLIELQHELISQTWNPEPYLRIEITKNET | >PF_WP_03255 |
| | EKRKLGLLCIKDKIVQQAIKTAFEPQLEKTFLNLSYGYRPNKGPE | 6864.1 |
| | RAIKRVVHDLKKLKSGYVAKLDIDNYFDTINHERLFTRLANWLKD | [Bacteroides |
| | DETLRLIRLCIQTGIVTPQLQWQEINKGVPQGAILSPLLANFYLH | fragilis] |
| | PFDQFAANKVPMYIRYADDFLIATSTEKQIKEAVELVKEELESQF | |
| | YLQLNTPIIHNFHDGIEFLGITI | |
| 8388 | GVDGKKALEPSQRLALYEVLVKNWKQWKHQPLKRVYIPKADGTRR | >DS_N.sp.I1/BA |
| | GLGIPTISDRAYQCLIKYALEPAAEAMFNARSYGFRPGRSCHDVQ | 000019/ |
| | KLLFSNLNGGQANGLSKRILELDIERCFDKIDHKFLMQSVQLPKA | 6209592 . . . |
| | AKQGIFWAIKAGVRGEFPSSESGTPQGGVISPLLANIVLHGLENV | 6207287/ |
| | GHELRYKVRSGGRQIDTIKGFRYADDVVFLLKPEDNPEALRQNID | Nostoc sp./CL2 |
| | TFLEARGLKVKEAKTKIVHSTDSFDFLGWNF | |
| 8389 | GVDGKASLTYKERVELDKLLMEQVNTWTHSKLREIPIPKKDGTKR | >DS_cianobacteria |
| | ILKVPTIKDRAWQCIIKYTIEPAHEAIFHERSYGFRPGRSTHDAQ | fid\|115549836\|locus |
| | KYLFDNLRSQSHGKDKIILEMDIEKCFDRISHNHLMSQIIAPQSV | \|VBIAnaSp4 |
| | KLGVWKCLKAGVNPEFPEQGTPQGGVCSPLLANIALHGIEAIHKS | 9473_5321 |
| | VRYADDMVFIFKKGDDQAKVFDEITEFLRIRGLNIKTAKTRFVPA | [Anabaena |
| | TTGFNFLGWKF | sp. 90] |
| 8390 | GCGESRTPRFNREVRRIIPPIDSNQCLAKYALEPAHEATFHEHSY | >DS_cianobacteria |
| | GFRPGRSTHDAQSQIANYLASSKGGINKRILELDIEKCFDRINHS | fid\|22782216\| |
| | TIMSNLIAPQGLKQGIFRALKAGINPEFPEQGTPQGGVVSPLLAN | locus\|VBINosSp37 |
| | IALNGIEDLHQYHDCNYKKITPSTPERNIKKACVRYADDMVFFLR | 423_6520\| |
| | PEDDAEEILEKISQFLAQRGLKISEKKTKLTASTDGFDFLGWNF | [Nostoc |
| | | sp. PCC7120] |
| 8391 | GIDGIKSLNFKQRFALAERLLKAHDWKHSKLREIPIPKKDGTTRM | >DS_C.w.I6/NZ |
| | LKVPTMADRAWQCLVKYALEPAHEALFHARSYGFRPGRSTHDAQK | AADV02000041/ |
| | ILFLNLKSDSNGLNKRILELDIEKCFDRINHTSIMERVIAPQTIK | 1584 . . . 4153/ |
| | TGIWRCLKAGVNPEFPEQGTPQGGVVSPLLANVALDGIEDIHYSI | Crocosphaera |
| | RYADDMVVILKPKDDADKILKDIQEFLAARGLKVSEKKTKLVRAT | watsonii/CL2 |
| | EGFDFLGWHF | |
| 8392 | GIDGKKSLTFEERFALEELLKAKSSKWKHQKLRAIPIPKKDGTTT | >DS_cianobacteria |
| | RLLKIPTLADRCWQCLAKYALEPAHEATFHKHSYGFRTGRSAHDA | fid\|115603115\|locus |
| | QKQVFQNLKSSSNGINKRILELDIEKCFDRINHSSIISNLIAPNR | \|VBIRivSp7 |
| | LKLGIFRCLKVGINPDFPEQGTCQGGVVSPLLANIALNGIEELHK | 7222_2588\| |
| | YHTNKGRKIKATTPEKDINTACVRYADDMVFFLRPEDDEKEILDN | [Rivularia |
| | ISQFLAKRGLKVSEKKTKLTASTFGFDFLGWHF | sp. PCC7116] |
| 8393 | GIDGVKSLDFNGRFELEITLKQSSGNWHHQELREIPIPKKDGTTR | >DS_N.sp.I2/ |
| | MLKIPTIADRCWQCLAKYALEPAHEATFHARSYGFRTGRAAHDAQ | BA000020/ |
| | QFLFSNLSSKAKRISKRVIELDIEKCFDRINHSTIMENLIAPKGI | 259212 . . . |
| | KLGIYRCLKAGINPEFPEQGTPQGGVVSPLLANIALNGIESIHRY | 261419/ |
| | HKDNQRITNKTPESDIRYPSVRYADDMVIVLRPQDDANEILAKIE | Nostoc |
| | DFLNARGMKVSAKKTKITATTDGFDFLGWHI | sp./CL2 |
| 8394 | GIDGKKSLTFRERFELSELLKASCNNWKHQGLREIPIPKKDGTTR | >DS_cianobacteria |
| | MLKIPTMADRAWQCLAKYALEPAHEATFHARSYGFRSGRSAHDAQ | fid\|115514952\| |
| | TVLLTHLRSNNNGINKRVIELDIEKCFDRISHTSIMENLIAPKGV | locus\|VBICalSp2 |
| | KLGIFRCLKAGINPEFPEQGTPQGGVVSPLLANIALNGIESIHRY | 27687_3172\| |
| | HRNGSKITNKTAGKDITEPSIRYADDMVIIIRPQDDAQKILADID | [Calothrix |
| | SFLAARGMKVSEKKTKITAATDGFDFLGWHF | sp. PCC6303] |
| 8395 | GIDGKTALTFEQRFQLSEKLRTEANNWKHQGLREIPIPKKDGKTR | >DS_cianobacteria |
| | ILKVPTIADRAYQCLVKYALEPAHEATFHARSYGFRTGRSAQDAQ | fid\|115337801\| |
| | KYLYTNLNSSVNGIEKRVIELDIEKCFDRINHTAIMDRLIAPYSI | locus\|VBIAnaCyl |
| | RLGIFRCLKAGVNPEFPEQGTPQGGVVSPLLANIALNGIESIHRY | 106394_6267 |
| | HIQGLRITNKTKGYKIVEPSVRYADDMIIILRPEDDAKEILDKIS | [Anabaena |
| | RFLAERGMKVSEKKTKLTATTDGFDFLGWHF | cylindrica |
| | | PCC7122] |
| 8396 | GIDGKASLNHEERFALSEELRTRSSKWKHQKLREIPIPKKDGTTR | >DS_cianobacteria |
| | LLKVPTIGDRAWQCLVKLALEPAHEATFHAKSYGFRTGRAAHDAQ | fid\|115430450\| |
| | KYLFDHLRSTSHGIEKRVIELDIEKCFDRIAHKSIMERLIAPSGI | locus\|VBICriEpi2 |
| | KLGIYRCLKAGVNPEFPEQGTPQGGVVSPLLANIALNGIEDIHQS | 39080_1694\| |
| | VRYADDMVFILKPKDDAVAILEQISQFLAERGMKISEKKTKLTAT | [Crinalium |
| | TDGFDFLGWHF | epipsammum |
| | | PCC9333] |
| 8397 | GIDGRASLTFEERLALSEELRAKSNNWKHQKLRSIPIPKKDGSTR | >DS_cianobacteria |
| | LLKIPTIADRAWQCLAKYALEPAHEATFHARSYGFRTGRSAHDAQ | fid\|115683516\| |
| | KFLFLNLSSKAHGISKRVIELDIEKCFDRISHTSIMERLIAPKGI | locus\|VBIOscNig |
| | KTGIFRCLKSGVNPGFPEQGTPQGGVVSPLLANIALNGIEEIHRS | 7962_8018 |
| | VRYADDMVIILKPKDDAKAILDKVSEFLAARGMKVSEKKTKLTAT | [Oscillatoria |
| | TDGFDFLGWHF | nigroviridis |
| | | PCC7112] |
| 8398 | GVDGYTASKPNERIKLYQQLVKCNVFRHRPKPAKRTFIPKKNGKL | >DS_B.me.I2/A |
| | RPLGIPTMRDRVYQNVVKNALEPQWEVKFEPTSYGFRPKRSTHDA | F142677/ |
| | ISNLFNKLNTNSKKKWVFEGDFLGCFDHLNHNWIMEQTSMFPGNT | 34045 . . . |
| | LIKRWLNMGYIEQDMLHTTTEGTPQGGIVSPLLANIALCGMEEEI | 36400/ |
| | GIVYKKTYKSNGGYKIDPKKIGRVLYADDFVIVTETKEQAESMYQ | Bacillus |
| | NLTPYLRKRGITLSKEKTRVTHIEDGFDFLGFSL | megaterium/CL1 |
| 8399 | GIDGYISNTPQERVELFNKLSRYSVRNIKVKPARRTYIPKKNGKL | >DS_Bacillifid\| |
| | RPLGIPVIVDRVYQNAFKNALEPQWEAKFEMTSYGFRPKRSTHDA | 18918903\|locus\| |
| | MSDLFTKLSKGSAKGWIFEGDFEGCFDNLNHDYIMGCINNFPNKS | VBIBacCer120424 |
| | IIRDWLESGYVDNDVFNETTKGTPQGGIISPLLANVALHGMEKEI | _5584\|[Bacillus |
| | GVRYIHTTRQGDTLYSNSVGVVRYADDFVIVCPTEEEAYGMYDKL | cereus |
| | EPYLNKRGLNLAKDKTRVVHISKGFDFLGFNF | Q1] |
| 8400 | GIDGITTNTPEDRVKLFHLLKGYSVRNIKAFPVKRAYIPKKNGKK | >DS_B.a.I1/AE0 |
| | RPLGIPVIKDRIFQNMVKNALEPQWECRFESMSYGFRPKRSAHDA | 11190/ |
| | MANLFLKLSRGTNRAWIFEGDFQGCFDNLNHEHILSCIEGFPYSN | 6579 . . . 9109/ |
| | AINQWLNAGCIDNKTFYKTETGTPQGGIISPLLANIALHGMEKEL | Bacillus |
| | GVRYHFPKRDGAMLYPDSIGIVRYADDFVIVCNSKEEAESMYAKL | anthracis/ |
| | QPYLDKRGLKLAEEKTRVVHITDGFDFLGFNF | CL1 |
| 8401 | GVDGKKSLRPNQRLKLVNELRLKGYKAKALRRVWIPKPGRDEKRG | >DS_C.w.I1/NZ |
| | LGIPTMKDRAMQALVKSALEPYWEAQFEGTSYGFRPGRSAQDAIS | AADV01000039/ |
| | RIFLAIKTNAKYVLDADIAKCFDKINHDYLLSKVDCPHNIKRIIK | 6112 . . . 8597/ |
| | QWLECGVMDKGIFEETDSGTPQGGVISPLLANIALHGMIIDIENH | Crocosphaera |
| | FPRTKRREDGSLKQGYKPKIIRYADDFVILHTDYDVILQCKNLVA | watsonii/CL2 |
| | QWLEKVGLELKPEKTSIRHTLKSIVHNGKTIEPGFDFLGFNI | |
| 8402 | GIDGIKNLPSMQRFNLVDLLKRHRFKASPTRRVWIPKPGKDEKRP | >DS_Tr.e.I2/CP |
| | LGISTMYDRALQALVKLGRSPEWEAHFEPNSYGLRPGRSTHDAIA | 000393/5587083 |
| | AIYVSINKKPKYVLDADISKCFDRINHDALLRKIGRTPYRRLIKQ | . . . 5589603/ |
| | WLKSGVFDNKQFSDTLEGTPQGGVISTLLVNIALHGMEKCLEKYA | Trichodesmium |
| | ETLPGKKRDNKQALSLIRYADDFVILHEDIKVVMQAKTVIQEWLN | erythraeum/CL2 |
| | QVGLELKPEKTKIAHTLEEYEGNKPEFDFLGFNI | |
| 8403 | GIDGVKSLKPSARLTLVMNMKLNHKVKATRRVWIPKPGNVEKRPL | >DS_C.sp. I1/X7 |
| | GIPTMQDRATQSLVKLALEPEWEAKFEPNSYGFRPGRNAHDAREA | 1404/ |
| | IFNSIRYSNKWVLDADISKCFDKINHEKLLTKINTFPTMRRQIKA | 446 . . . 2898/ |
| | WLKAGVLDNGHFSETTEGTPQGGVISPLLANIALHGLEKLVKEFA | Calothrix |
| | ASQRGGKVKNQNSISLIRYADDFVILAPNKTQIIVLKEIVKTWLA | sp./CL2 |
| | EMGLELNPNKTRIVSTFKSSEIFASQEVGFNFLGFNV | |
| 8404 | GVDGRKNLSPKARLILVQSMKLGDKASPTRRVWIPKPGSSGEKRP | >DS_N.sp. I4/AP |
| | LSIPTLYDRALQSLVKLALEPEWEARFEPNSFGFRPGRNAHDAMK | 003604/ |
| | AIFNTIKFKPKYVLDADIAKCFDKIDHNVLLSKLNTFPTISRQIR | 45422 . . . 47908/ |
| | AWLKAGVIDFSEYALHTTSMGVPQGGTISPLLANIALHGMENRIK | Nostoc |
| | QVALTLPGCKSENRQAISLIRFADDFVILHKDLAVIQRCQQIISE | sp./CL2 |
| | WLSELGLELKPSKTRISHTLNMYEGKVGFDFLGFTV | |
| 8405 | GVDGVKSLTPKARLALTKNLRISEKAKPMRRVWIAKPGTQEKRPL | >DS_G.v.I1/BA |
| | GIPTMTDRARQALLTLALEPEWEARFEPNSYGFRPGRSCHDALQA | 000045/ |
| | IYNAIRQQSKFVLDADIAKCFDRIDQQALLKKMNTSSAIRRQIRA | 168850 . . . |
| | WLKAGVMEGSELFPTPTGTPQGGVISPLLANIALHGMEERVKQVS | 171364/ |
| | KMAQLIRYADDFVCIHTDQQIVQSCQTVLEEWLAGMGLELKPSKT | Gloeobacter |
| | RIAHTLLLEEGQPGFDFLGFTV | violaceus/CL2 |
| 8406 | QRCFLSLAKRSSAEWILEGDIRACFDAFDHDWLIEHTPTDQGRLR | >DS_Gfid\|11564 |
| | AWLKSGFMEQRRIFPTERGTAQGGIISPTVANMVLDGLEGRIRAR | 1574\|locus\|VBIT |
| | FKRRGKVNLIRFADDFVITGESRAILENDVTPLVTEFLHERGLVL | hiNit264030_3543\| |
| | APEKTRIVHIDDGFDFLGFRF | [Thioalkalivibrio |
| | | nitratireducens sp. |
| | | DSM14787] |
| 8407 | IDRTQQALHLLALDPISETIADPNSYGFRPNRSTADAIAQCFKCL | >DS_fid\|352979 |
| | CQKRSARWVLEEDLKACFDKIGYQWLIENIQIDKRMLKQWLGSDF | 35\|locus\|VBIXen |
| | IDKGLFYRTAEGTPQGGIISPTLMLLTLAGLEKRVKEVARKTDDR | Bov95754_1334\| |
| | INSIEYADNFVMTGASEDVLLNEVKPQLIDFLRERGLTLSEEKTH | [Xenorhabdus |
| | ITHINDGFDFLGFNL | bovienii |
| | | SS2004] |
| 8408 | GIDGIIWNSDARCMTAVNQLSRKGYHAKPLRRIYIPKKNGKLRPL | >DS_fid\|541836 |
| | GIPCMIDRAQQALHLLALEPISETVADLNSYGFRPNRSAADAIAQ | 25\|locus\|VBIShe |
| | CFKCLCMKRSSQWVLEGDIKACFDKIGHQWLIDNIQLDKRMLKQW | Bal163160_2541\| |
| | LGCGYVDKGLFYKTAEGTPQGGIIPPTLMLLTLAGLEQLVKSIAC | [Shewanella |
| | KTGNSVNFIGYADDFIITGSSKEVLVNEIKPQLIGFLQERGLTLS | baltica OS117] |
| | DDKTHITHIDDGFDFLGFNI | |
| 8409 | GIDGIIWNTDARRMKAVNQLSRKAYIAKPLKRIYIPKKNGKLRPL | >DS_fid\|589338 |
| | GIPCMIDRAQQALHLLALEPVSETLADPNSYGFRPNRSTADAVDQ | 41\|locus\|VBIShe |
| | CFKCLAQKKSAQWVLEGDIKACFDKIGHQWLLDNITVDKRMLEQW | Bal147952_0958 |
| | LKSGFMDKGLFYRTDEGTPQGGVISPSLMLMTLAGLEQHIKSTAL | [Shewanella |
| | KKGTRANFIGYADDFVVTCASKEVLENDIKPLITDFLAERGLTLS | baltica |
| | EEKTHITHINDGFDFLGFNH | OS678] |
| 8410 | GIDGVIWNTDARRIAAVKQLKRKAYQAKPLKRIYIPKKNGKLRPL | >DS_Sh.sp. I1/C |
| | GIPCMIDRAQQALHLLALEPISETVADPNSYGFRPHRSTADAIAQ | P000446/ |
| | CFLCLSQRYSSEWVLEGDIKACFDKIGHQWLIDNIALDKKMLRQW | 2526748 . . . |
| | LECGFMDKGLFYRTDEGTPQGGIISPTLMLLTLSGLEQLLKATAR | 2528903/ |
| | RKGCNVNFIGYADDFVVTGSSKEVLVNEIKPLIARFLAERGLTLS | Shewanella |
| | EEKTHVTHINDGFDFLGFNL | sp./CL1 |
| 8411 | GIDGEKWLSSASKMKAVLSLTGKRYKAKPLKRVFINKPGKTKKRP | >DS_Ms.b.I1/N |
| | LGIPTMYDRAIQSLYSLALEPVAEIKSDLRSFGFRKHRSTKDACQ | Z_AAAR02000002/ |
| | QIFLCLSKKTSAQWILEGDIRGCFDNINHQWLLTNIPIDKAILTQ | 377828 . . . |
| | FLKAGFIYKRHLNPTKAGTPQGGIISPILANMTLDGIEKMLLVKY | 379992/ |
| | PKKGKNSKKVNFIRYADDFIVTANSKETAGEIKDEVVAFLKERGL | Methanosarcina |
| | ELSDDKTFITNINEGFDFLGWNF | barkeri/ |
| | | CL1 |
| 8412 | GVDKELWSTTASKMQAVLSLTDKNYKAKPLRRVYIEKKGKKAKRP | >DS_clostridiafid |
| | LGIPCMYDRAMQALYALALDPVSEVTADTKSFGFRKNRCCQDACE | \|161805880\|locus\| |
| | YIFTALSRENCAKWILEGDIKACFDYISHEWLIENIPMDKSVLKQ | VBICloPas18 |
| | FLKAGFVFENELFPTDDGTPQGGVISPILANMALDGMQKALSDRF | 034_1667 |
| | HTNKLGRVDNRFQIANKVYLVRYADDFIVTAATKEIAEEAKELIR | [Clostridium |
| | EFLQTRGLELSEEKTKITHINDGFDMLGWTF | pasteurianum |
| | | BC1] |
| 8413 | GIDGELWTTPAQKMEALLSLTDKGYKASPLRRVYIDKKGKKKKRP | >DS_Bacillifid\| |
| | LGIPTMYDRAMQALYALALEPIAETTADTKSFGFRKGRSCQDACE | 19653441\|locus\|V |
| | YIFTALSRKASPQWILKGDIKGCFDNISHDWLLENIPMDKSILKQ | BIStrEqu35012 |
| | FLKAGFVFKGELFPTEDGTPQGGIISSILANMALDGLQQVLSDRF | 1915 |
| | HTNRLGRIDFRFKNSHKVNLVRYADDFIVTAATQEIALEAKELIR | [Streptococcus equi |
| | EFLIGRGLELSEEKTLVTHINDGFDLLGWNF | sub sp. |
| | | zooepidemicus] |
| 8414 | GVDGQLWTNPPRKRQAIDELRSRGYRPQPLKRIYIPKRNGKQRPL | >DS_Bacteroidet |
| | SIPTMKDRAMQALHLMALQPVSETTADPCSFGFRPARQVADAVER | esfid\|115626437\| |
| | CFGLLSRQDSPQWVLEADIEACFDRIDHDWLLQHIPMEKTILGQW | locus\|VBIFibAes |
| | LKAGYIEKGNWWPTTEGTPQGGIISPVLANMALDGLAKELAAHFA | 90597_0767\| |
| | KSYKRPDRGFNPKVRLVRYADDFIITGISRQQLEEQVKPVVCNFL | [Fibrella |
| | SKRGLRLSESKTRQTAITEGFDFLGFTF | aestuarina] |
| 8415 | GVDGKIWSTPVAKSTGAQALQHRGYRPQPLRRIYIPKSNGKKRPL | >DS_fid\|199617 |
| | GIPTMRDRAMQALWKLALEPVAETRADPNSYGFRPQRSTADAIAH | 45\|locus\|VBIPse |
| | CENALAKRGSAHWVLEADIRGCFDNISHDWLLTNVPMDKVVLRKW | Stu31643_0668\| |
| | LRAGYVDQGALFATEAGTPQGGIISPVLANWTLDGLEDVVHASVA | [Pseudomonas |
| | STARKRKPFKIHVVRYADDFIITGATKAVLQHQVRPAIEAFLKER | stutzeriA1501] |
| | GLELSDEKTQITHISQGFDFLGQNV | |
| 8416 | GVDGKIWATPAAKSSGMESMRHRSYRALPLRRIYIPKSNGQKRPL | >DS_P.p.12/Y18 |
| | GIPRMLCRSMQALWKLALEPVSESLADPNSYGFRPNRSTADAIEY | 999/ |
| | CFITLAKRTSPVWVLEGDIRGCFDNFNHEWMLKNIPMDKTILRRW | 752 . . . 2957/ |
| | LQAGFIDEGTLFATQAGTPQGGIISPVIANMALDGLEAAVHASVG | Pseudomonas |
| | PTKRARERSKINVVRYADDFVVTGISKEILEHSVLPAVRQFMAIR | putida |
| | GLELSEEKTKITHIAEGFDFLGQNV | /CL1 |
| 8417 | GVDKVVWDTPEKKLCAMGDLKRRGYRPKPLKRVHIPKANGKLRPL | >DS_fid\|423466 |
| | GIPTMKDRAMQALYLLGLLPVSETTADGCSYGFRPERSVADAIER | 03\|locus\|VBIGa |
| | CFNALGRRDAAAWVLEADIKGCFDHISHDWLLGNVPMDKRVLATW | mPro61291_1949\| |
| | LKCGFMEKAVWFATEAGTPQGGIISPTLANFALDGLEQLLSKTFY | [gammaproteo |
| | RTMRHGKMVHPKVHLIRYADDFVITGSSEELLVNEVKPLVERFLA | bacterium |
| | ERGLMLSAEKTKVTHIDEGFDFLGQNV | sp. |
| | | HdN1] |
| 8418 | GVDRVTWSTPETKSEAVLSLRRHGYRPRPLRRIYIPKANGKKRPL | >DS_fid\|211635 |
| | GIPTMRDRAMQALYLLALEPIAETTGDKDSYGFRPGRSVADAIRQ | 95\|locus\|VBIBor |
| | CHTVLAWKRSAEWVLEADIEGCFDNISHDWLAENIPMDKAILKSW | Pet31633_1067\| |
| | LKAGYVESGSLFPTEAGTPQGGIISPVLANMALDGLQEVLGKSFF | [Bordetella |
| | RTRRQNKHYDPKVNFVRYADDFIVTGYSRELLEIEVLPLVEKFLA | petrii |
| | ARGLNISKAKTRVTHISEGFDFLGKNI | DSM128041 |
| 8419 | GVDGKTWSKPGSKMKAIYTLKRRGYKPLPLRRIYIPKSNGKKRPL | >DS_fid\|485791 |
| | GIPTMKDRAMQALYLMALEPVAETTADPNSFGFRPCRSTADAIEQ | 26\|locus\|VBIEsc |
| | CFTTLHRADRAQWILEADIRSCFDEISHEWLIANIPTDTAILKRW | Col159162_5518 |
| | LKAGYIDLGKLYPTSAGTPQGGIISPTLANMVLDGLQPLLKKTFY | [Escherichia coli |
| | RGGLNPEKINIIRYADDFVITGISHDTLSEKVLPLLENFLAERGL | UMNK88] |
| | TLSPEKTRITHISDGFDFLGMNI | |
| 8420 | GVDGITWSTQEQKTQAIKSLRRRGYKPQPLRRVYIPKANGKQRPL | >DS_Th.e.I1/ |
| | GIPTMKDRAMQALYALALEPVAETTADRNSYGFRRGRCTADAAGQ | BA000039/ |
| | CFLALARAKSAEHVLDADISGCFDNISHEWLLANTPLDKGILRKW | 27344 . . . 30566/ |
| | LKSGFVWKQQLFPTHAGTPQGGVISPVLANITLDGMEELLAKHLR | Thermo |
| | GQKVNLIRYADDFVVTGKDEETLEKARNLIQEFLKERGLTLSPEK | Synechococcus |
| | TKIVHIEEGFDFLGWNI | elongatus/ |
| | | CL1 |
| 8421 | GVDGETWSTPESKWKAIFRLQRTGYRPRPLRRVYIPKANGQRRPL | >DS_A.v.I1/AY |
| | GIPTMLDRAMQALYLLALEPVSETTADRNSYGFRPHRSTADAIEQ | 057439/ |
| | LFVNLGRKHSAQWVMEGDIKGCFDNISHDWLIANVPLDKAVLRKW | 1648 . . . 4444/ |
| | LKAGYLESGQLNPTGAGTPQGGIISPVLANLALDGLEKALESRFG | Azotobacter |
| | QRNTKASYKTKVNYVRYADDFVITGISKELLVNEVKPVVAAFMAE | vinelandii/ |
| | RGLSLAAEKSLFTHVSEGFDFLGQNV | CL1 |
| 8422 | GVDGQTWSSPEVKFLAINLLKRRGYKPQPLKRVYIPKSNGKSRPL | >DS_E.c.I5/AF0 |
| | GIPTMKDRAMQALYLLALEPVAEVTADQRSFGFRTGRSTADAIAQ | 74613/ |
| | CFCVLAQKTSAEWVLEGDIRGCFDNISHQWLIDNTSTDRQILTKW | 58241 . . . 60646/ |
| | LKAGYREKGQLFPVNSGTPQGGIISPVLANIALDGLEALLASEFK | Escherichia |
| | KRTVKGRLVNPKVNYVRYADDFIITGESKELLESQVLPVVRRFMA | coli/CL1 |
| | ERGLMLSPEKTKITHIEEGFDFLGQNI | |
| 8423 | GVDGITWSTPEAKSQAMLSIKRRGYRPQPLKRVYIPKTNGKMRPL | >DS_fid\|867388 |
| | GIPTMKDRAMQALYLLALEPVAETTADGRSFGFRPERSTADAIEQ | 73\|locus\|VBIPse |
| | CFTTLSKKVAPQWILEGDIKGCFDNISHDWLMGHVPTDREILRKW | Aer240047_2455 |
| | LKAGYMEDRQLFPTEAGTPQGGIISPTLANLVLDGLEAKLDAAFG | [Pseudomonas |
| | RKRYANGVQTRLMVNYVRYADDFIVTGRSKELLEQEVMPIIKDFM | aeruginosaDK2] |
| | QERGLTLSPEKTKITHIDDGFDFLGQNV | |
| 8424 | GIDGITKEDYGKKLKANLLSLLTRIRKGQYQAKPARIVKIPKEDG | >DS_fid\|228289 |
| | GKRPLVISCFEDKIIESTVSKILNSVFEPIFLKYSYGFHPKLNAH | 08\|locus\|VBIOri |
| | DALRELNRLTYNFNKGAIVEIDITKCFNTIKHCELMEFLRKRISD | Tsu129072_1468 |
| | KKFLRLVMKLIETPIIENDTIVTNKEGCRQGSIVSPILANVFLHY | [Orientia |
| | VIDSWFAKISEENLIGQTGMVRYCDDMVFVFESEADAKRFYDVLP | tsutsugamushi |
| | KRLNKYGLNINEAKSQMIKSGRDHAAN | str. Ikeda] |
| 8425 | GIDGVTKEVYGKKLEDNLQDLLARIRRHAYTPQASRLVEIPKEDG | >DS_fid\|352902 |
| | STRPLAISCFEDKIVQMAVTKLLTAIYEPLFLPCSYGYREGKNGH | 99\|locus\|VBILeg |
| | EALRALMKYSNEFRKGATLEIDLRKYFNTIPHGKLLEILEKKITD | Lon159544_1142\| |
| | RRFLKLIRKLIRSPVVANGKAELNELGCPQGSIISPILSNIYLHS | [Legionella |
| | VVDSWFDEISKSHLIGKTAMVRFADDMVFLFQRSEDAEKFYKVLP | longbeachae |
| | KRLEKYGLQLHVDKSSLLKSGSKEAEEADTRGERLQTYKFLGFTC | NSW150] |
| 8426 | GIDRMTKAAYGEHLDGNIHNLILRIRRGTYRPKAARITQIPKEDG | >DS_fid\|424648 |
| | SKRPLAISCTEDKLVQLAVSDILSRIYEPLFLPCSYGFRPGLNCH | 02\|locus\|VBIXen |
| | AALKALQQQTYRNWNGAVVEIDIRKYFNTIPHIELMSLLRKKISD | Nem38452_2364 |
| | RRFLRLIEVLITAPVIEGKQVSENVRGCPQGSILSPVLANIYLHQ | [Xenorhabdus |
| | VIDEWFDEISRSHIHGRAEMVRYADDRVFTFEFMSEAERFYKVLP | nematophila |
| | KRLNKYGLELHDDKSQRIPAGHIAALRASQSGRRLPTFNFLGFTC | ATCC19061] |
| 8427 | GVDGVTKAEYQENLETNLQNLHLKLRQMSYRPQPVRQVEIPKEDG | >DS_Ac.ma.I1/C |
| | SMRPLGISCTEDKVVQEMTRRILEAIYEPVFIDTSYGFRPKRSCH | P000840.1/ |
| | DALRQLNREVMRKPVNWVADIDLAKFFDTMPHQEILSVLSIRIKD | 228971 . . . 230873/ |
| | GNLLRLIARMLKAGIQTPGGVVYDELGSPQGSIVSPVIANIFLDY | Acaryochloris |
| | VLDQWFTNVVRHHCRGYCAIIRYADDVAAVFEHEEDAIRFMRVLP | marina/BacterialE |
| | RRLEKYGLRLNTKKTHLLAFGKRNARRCFQTGQRPSTFDFLGLTH | |
| 8428 | GIDRQTAKDYEANLEVNLKSLLERIKSGRYKAPPVRRTYIPKADG | >DS_fid\|426856 |
| | SQRPLGIPTFEDKVAQRAIVLLLEPIYEQDFRPFSFGFRPGRSAH | 79\|locus\|VBISti |
| | QALRELRSSILERNGRWVLDVDLRRYFDTIEHGKLREVLARRVAD | Aur4371220374 |
| | GVVRRMIDKWLKAGVLEEGPLLRLEQGTPQGGVISPLLANVYLHY | 7_3158\| |
| | VLDEWYEREVVPRMKGKCSLIRYADDLVMVFEDFLDCRRVLEVLG | [Stigmatella |
| | KRLAKYGLTLHPGKTRMVDFRFKRPGGGQHPATQATTFDFLGFTH | aurantiaca |
| | | DW4/31(Prj: |
| | | 54333)] |
| 8429 | GIDGRTADDYEKDLEANLESLRIRMMSGSYRAPPVRRHYIPKADG | >DS_fid\|190138 |
| | SRRPLGIPTIEDKVAQRAIVMLLEPIYEEDFLDCSFGFRPERSAH | 491\|locus\|VBIRh |
| | DAIRTLRDGIMDTGQRWVIDADISKYFDSIDHGHLRSFLDLRIRD | iEt1298076_5694 |
| | GVIRRMIDKWLNAGVLDQGTSSRSVAGTPQGGVISPLLANILLLH | [Rhizobium etli |
| | VLDRWFVEVVKPRLKRRCQMVRYADDFVMSFEDHLDGRRMLAVLG | bv.mimosae |
| | KRFERYGLRLHPDKTRYVDFRFRRPHG | str. Mim1] |
| 8430 | GVDEETWIDYHKQRETRIPQLLAAFKSGNYRAPNIRRVYIPKDKG | >DS_Bacteroidet |
| | KLRPLGLPTVEDKVLQTAVTRVLRPVYEDIFYHSSYGFRPGKSQH | esfid\|61290805\|locus |
| | QALEELTRQVSLEGKRYIIDADMQNYFGSINHQCLRDLLDLRIKD | \|VBINiaKor |
| | GVIRKMIDKWLKAGILDNGQLVYPTEGTPQGGSISPLISNVYLHY | 154066_6177\| |
| | VLDEWFYQQIRPLLKGDSFLIRFADDFLLGFTNKEDALRVMHVLP | [Niastella |
| | KRLGKYGLMLHPEKTKLIDLTTKKGGPDQEKNTFDFLGFCH | koreensis |
| | | GR2010] |
| 8431 | GVDGITKEQYGQDLEHNVRDLHARMKSMRYRHQPIRRVHIPKERG | >DS_fid\|236591 |
| | KTRPIGISCTEDKIVQAAVREMLEVIYEPVFRDVSYGFRPGRSAH | 27\|locus\|VBISor |
| | DALRALNRMLLGGVEWILEADIESFFDSIDRTKLMEMLQARVADK | Cel80414_0791\| |
| | SLLRLVGKCLHVGVLDGAEFYAPEDGTVQGSVLSPLLGNVYLHHV | [Sorangium |
| | LDLWIEREVQPRLVGKATLIRYADDFIIGFEREDDAKRVTEVLPR | cellulosum |
| | RFERYGLKLHPDKTRLLPFGRPDNGQPGGKGPATFDFLGFTH | Soce56] |
| 8432 | GTDGKSWKTYEAQLEERLPKLHEEIHTGSYRAQPVKRVYIPKTDG | >DS_Chlorobifid |
| | QKRPLGITAIEDKLVQQAVVTVLNQIYETEFYGFSYGYRPGRAPE | \|21392973\|locus\| |
| | NALDALATAILKRPINWILDADLQKFFDSIPHDKLMALISIRVGD | VBIChlPha1221 |
| | KRILRLIGKWLKTGYIEDGKRYRQTEGTPQGSVISPLLANIYLHY | 04_2646 |
| | VVDEWVEQERRRRNNGEVIIIRYADDLVLGFQYKTEAERYLEALS | [Chlorobium |
| | ERVQTYGLKLHPEKTSLKEFGRYAEERRRKRGEE | phaeo |
| | | bacteroides |
| | | DSM266] |
| 8433 | YGEELDARLLDLQDRILRGSYHPQPVRRVHIPKGSGTRPLGIPAL | >DS_fid\|236778 |
| | EDKIVQQAVRRGLELIYESMFLGFSYGFRPRRSTHDALDALAVAI | 55\|locus\|VBISor |
| | GKRKVNWIVDADIRAFYDTIAHAWMQRFIEHRIGDRRLVRLLMKW | Cel80414_10115\| |
| | LHAGVMEDGVLHEVDEGTPQGGIISPLMANIYLHYVLDLWAHAWR | [Sorangium |
| | KRHARGEVYIVRYADDVVMGFEDGRDARSMRAALSKRLASFGLEL | cellulosum |
| | HPDKTRVLFFGRYAYEKCERRGLRKPATFDFLGFTH | Soce56] |
| 8434 | GVDGVTWQSYEVGLGSNLRDLHRRVHTGSYRALPVLRRYIPKADA | >DS_Bfid\|45180 |
| | GLRPLGVAALEDKLVQSVMVEVLNAIYEEDFLGFSYGFRPGRNQH | 964\|locus\|VBIBu |
| | DALDALAAAIQWRPVNWILDADIRSFFDTVNRQWLIRFVKHRVAD | rRhi170666_033 |
| | PRVIRLIGKWLDAGVLDNGRLMSVQAGTPQGSVICPLLANIYLHY | 1\|[Para |
| | VFDLWIERWRRQRARGTVVVSRYADDTVVGCQHEADALRLMKELR | Burkholderia |
| | QRMEEFDLTLHPEKTRVLEFGRYAAERRRRKGMGKPQTFAYLGFT | rhizoxinica |
| | H | HKI454] |
| 8435 | GVDEMTWRKYKEGSPGRIADLNERVHTGSYRAKPVRRSYINKSDG | >DS_UB.I1/AY |
| | RKRPLGVTALEDKIVQQAVSTILNQIYETDFMGFSYGFREKRSQH | 691909/ |
| | NALDALYIGISRRKINYILDADISGFFDKINHDWLLKFLEHRVAD | 2430 . . . 4342/ |
| | RKILRLIKKWLKVGVIEDGKRTSLEVGTPQGSVISPVLANVYLHY | uncultured_ |
| | AQDLWAHQWRKRHADGDVIIVRYADDSVVGFQYRKDADRFLKDLI | bacterium/Bacterial |
| | ERMGQFGLELHPVKTRLIEFGRFAVVNRRKRGERKPETFDFLGFT | E1 |
| | H | |
| 8436 | GVDGMSWREYEEDLHQRVGKLHARLHRGAYRATPSRRVYIPKADG | >DS_A.v.I5/CPO |
| | RQRPLGIASLEDKIVQQAVVTVLNAIYEEDFQGFSYGFRPGRSQH | 01157/ |
| | DALDALTVALKSQKVNWILDADITSFFDEIDHEWMLMFLGHRIAD | 2471407 . . . |
| | RRMLGLICKWLQAGVMEDGRRLAATKGTPQGAVISPLLANIYLHY | 2473316/Azotobacter |
| | VLDLWARQWRQRHARGEMIVVRYADDSVVGFRTQWQAQRFLVQLQ | vinelandii/ |
| | ERMARFGLSLNASKTRLIEFGRFAVQNRRRQGLGKPETFDFLGFT | BacterialE1 |
| | H | |
| 8437 | GVDGMTWQDYEEDLEPRLADLHKRVQRGTYRPQPSRRTYIPKADG | >DS_B.j.I2/BA0 |
| | KQRPLAIAALEDKIVQGATVIVLNAIYEGDFCGFSYGFRPGRGPH | 00040/ |
| | DALDALCTAIETRQVNWIIDADIQNFFGAVSQPWLVRFLEHRIGD | 2069342 . . . |
| | KRIIRLIQKWLKAGILEDGVVTADDRGTGQGPVISPLLGNIYLHY | 2071253/ |
| | ALDLWAKRWRQREVSGGMIIVRYADDVVVGFEREDDARRFLDAMR | Bradyrhizobium |
| | ARLEEFELTLHPAKTRLIEFGRHAAAQRKQRGLGKPETFAFMGFT | japonicum/ |
| | F | BacterialE1 |
| 8438 | GVDGVTWHDYEQDLDRNLEDLHGRLRRQAYRALPSRRRYIPKADG | >DS_Bfid\|19071 |
| | KQRPLGIAALEDKIVQRALVAVLNAVYEMDFLGFSYGFRPQRSQH | 807\|locus\|VBIBur |
| | DALDALATGIARTSVSWILDADISRFFDTVDHDWLIRFVEHRIGD | Cen118154_0098\| |
| | QRVIRLIRKWLKAGAMEDGVIEPTDEGTPQGSVISPLLANIYLHY | [Burkholderia |
| | VFDLWANQWRKRHAEGNVVIVRYADDVVVGFDKPHDAKRFRRAMQ | cenocepacia |
| | QRLEQFGLSVHPEKTRLIEFGRFAARNRASRGLGKPETFNFLGFT | J2315] |
| | H | |
| 8439 | GVDGIRWMDYAGNMKNNITDLHRRLHQGSYRAQPGRRHYIPKADG | >DS_S.ma.I1/B |
| | KQRPLGIASLEDKIVQYALVKILNAVYENDFMGFSYGFRPGRSQH | X664015/172056 |
| | DALDALATGLVRTNVNWVLDADISQFFDRVSHEWLIRFTEHRIGD | . . . 173964/ |
| | RRVIRLIRKWLTAGTSEEGQWRATEEGTPQGAVISPLLANIYLHY | Serratia |
| | VFDLWAHQWRRRYATGNVVMVRYADDIVIGFDKRYDARRFRIAMQ | marcescens/ |
| | RRLREFGLTVHPEKTRLMEFGRFAAENRAIRGKGKPETFNFLGFT | BacterialE1 |
| | H | |
| 8440 | GVDGITWKDYGEGLEENLADLHRRIHTGAYRAQPSRRKYIPKANG | >DS_Ch.ph.I2/C |
| | QQRPLGIAALEDKIVQRAVVAILTPIYEAEFLGFSYGFRPGRSQH | P000492/ |
| | DALDALAYGIKVKKIGWVLDADISRFFDTISHEWMIRFLEHRIGD | 3012641 . . . 3014550/ |
| | KRIVRLIIKWLKAGVLEDSVRIEAEEGTPQGAVISPLLANIYLHY | Chlorobium |
| | AYDLWAKQWREKHCKGDMIVVRFADDSVAGFQNKEDGERFLADLK | phaeobacteroides/ |
| | ERLAKFALTLHPEKTRLIEFGRYAAKNRQRRGQGRPETFDFLGFT | BacterialE1 |
| | H | |
| 8441 | GVDGVTWEQYAGNLEANVRDLHTRLHRGAYRARPSRRAYIPKADG | >DS_fid\|426823 |
| | RQRPLGIAALEDKLVQRAVVEVLNAVYETDFLGFSYGFRPGRSQH | 69\|locus\|VBISti |
| | QALDALSAGIYLKKVNWVLDADIRGFFDAIDHGWMQKFLEHRIED | Aur4371220374 |
| | TRLLRLVQKWLAAGVMEDGKWTQSKEGTPQGATVSPLLANLYLHY | 7_1515\| |
| | VFDLWSQRWRKRVARGEVIIVRYADDFVVGFQHRSDAERFWRELR | [Stigmatella |
| | ERLRSFALELHPEKTRLIEFGLYVAERRRERDQGRPETFNFLGFT | aurantiaca |
| | H | DW4/31(Prj: |
| | | 54333)] |
| 8442 | GVDGVTWTDYGQDLEANLQDLHVRVQSGCYRATPSRRAYIPKADG | >DS_Fr.sp. I4/CP |
| | RLRPLGIASLEDKIVQRAVVEVLGAVYEVDFRGFSYGFRPGRGPH | 000820/1651830 |
| | DALDALAVGIWRKRVNWVLDADIRDFFGQIDHSWLRRFLEHRIAD | . . . 1653736/ |
| | KRVLRLIDKWLAAGVVEDGEWTACEEGSPQGASVSPLLANVYLHY | Frankia sp. |
| | VLDLWVDWWRRRHARGDVIVVRWADDFIVGFEYEEDARRFLDELR | /BacterialE1 |
| | ERFAKFGLELHPDKTRLIEFGRYAARDRKRRGLGKPETFDFLGFT | |
| | H | |
| 8443 | GVDEITKKEYERNLEQNIDDLVERLKRKSYKPQPSIRVYIPKSNG | >DS_Co.ca.I1/F |
| | KLRPLGIACYEDKIVQLALKKILEAIYEPRFLNCMYGFRPNRGCH | P929038.1/ |
| | NAIKELYKRLNNTKICYIVDADIKGFFDHMKHEWIIKFLKLYIKD | 3172164 . . . |
| | PNIIGLVKKYLKVGVMDNGELMVNEEGSAQGNIISPILANIYMHN | 3174036/ |
| | VLTLWYKFIITKECKGDNFLIAYADDFVAGFQCKWEAENYYKLLK | Coprococcuscatus/ |
| | ERMEKFGLQLEDSKSRLLQSGAYIARAKQKSGECIRLQTFDFLGF | BacterialE |
| | TF | |
| 8444 | GIDRVTKVEYGANLEENISGLVIRLKNKSYKPLPVLRVFISKGNG | >DS_clostridiafi |
| | KMRPLGIAAYEDKFVQLAIKKILEAIYEPRFLENMYGFRPRRGCH | d\|58517021\|locus |
| | NAIKAAYDRIYENKINYIVDADIKGFFDNMSHEWIMKFLGVYISD | \|VBICloBot1808 |
| | PNFLWLINKYLKAGVMTDGTLIDSISGSAQGSIISPVIANVYMHN | 36_2089\| |
| | VLMLWYKFIVLNGIKGKSFLVTYADDFIAGFQYKWEAEKYYIELK | [Clostridium |
| | RRMAKENLELEDSKSRLLEFGRFAEGNRKARGEGKPETFDFLGFT | botulinum |
| | F | H04402065] |
| 8445 | GIDGITMPAYQQQLVGNITRLSDALKHKRFRANDIKRVFIPKANG | >DS_Ps.tu.I1/A |
| | KQRPLGLPTVDDKLVQQGVSQILQSIWEADFLPNSYGYRPNKSAH | AOH01000003/ |
| | QALHSLALNLQFKGYGYIVEADIKGFFNNLDHNWLMKMLKQRIDD | 353461 . . . 355380/ |
| | KAMLSLISQWLKARIKSPEGVFEYPKSGTPQGGIISPVLANIYLH | Pseudoalteromonas |
| | YALDLWFEKKVKPRMRGRAMLIRYADDFVCAFQYANDAERFYEVL | vtunicata/ |
| | PKRLKKFNLEVAEEKTSLLRFSRFHPSRKRQFVFLGFAF | BacterialE2 |
| 8446 | GVDKVTAKEFAEELKQNIENLAEHLEKKRYRAKLLRRVDIPKGEG | >DS_clostridiafi |
| | KTRPLGIPAIADKLVQSAAAKILEAIYEQDFLASSYGYRPKVSAH | d\|115615051\|locus\| |
| | TAIKDLSKELNYGDYSYIVEADIKGFFQNIDHAWLIRMLEQRIDD | VBIDehSp22 |
| | KAFVGLIKKWLKAGILKQDGEVEHPITGSPQGGIISPILANTYLH | 8777_0955 |
| | YVLDLWFEKIVKPNCEGEAYLCRYCDDFVCAFQYKGDADKFYRSL | [Dehalobacter |
| | PKRLEKFGLELAVDKTQIIQFNRWLRKQSSSFEYLGFEF | sp. CF] |
| 8447 | ERLKTKRYRTKLVRRCYIPKENGQERALGIPALEDKLVQLACAKL | >DS_fid\|115643 |
| | LTAIYEQDFLPVSYGYRPGRDAKEAVGDLGFNLQYGRFGHVVEAD | 628\|locus\|VBITh |
| | IQGFFDHLDHDWLLRMLALRIDDRAFLHLIRKWLKAGILDTDGQV | iNit264030_1141\| |
| | LHPDAGTPQGGIVSPILANVYLHYALDLWFERVVRPRCRGQALLI | [Thioalkali |
| | RYADDYVCAFQYREEAEGFYRVLPKRLAKFGLAVAPEKTRILRFS | vibrio |
| | RFHPGLPRRFAFLGFEL | nitratireducens |
| | | DSM14787] |
| 8448 | GIDWVTVEAYGENLKERLEGLVDSMKGKQYQPQPVRRVYIPKAGS | >DS_fid\|240262 |
| | KEKRGLGIPSTEDKLVQIMLKKILENIYEANFLDSSYGFRPGRNC | 34\|locus\|VBIWol |
| | HQTVNALDKAVMYKPINYIVEVDIKKFYDNIQHKWLMRCLRERIT | End95846_0368\| |
| | DPNLLWLIKRFLKAGIVEAGYYEATKQGTPQGGIVSPVLANIYLH | [Wolbachiaendos |
| | YVLDLWLEKKFKPRSRGYIQLIRFCDDFVVCCESKVDAEEFLELL | ymbiont of |
| | KQRLNKFGLEVSENKTRVVKFGKREWQQ | Culex |
| | | quinquefasciatus |
| | | Pel] |
| 8449 | GIDGISKEQYGANLDENIKELSSRLRNMGYRPQPKRRTYIPKPGS | >DS_fid\|115349 |
| | VKGRPLAISCFEDKLVELAIKRVLEPIYEVQFEDSSYGYRPGRSQ | 385\|locus\|VBITh |
| | HQCLDDLGRTIQQSRINTIVEADIRSFFNTVDHAWMLKFLGHRIG | iMob160332_04 |
| | DPRIIRLIGCLLKGGILEDGLVQASEEGTPQGSILSPLLSNIYLH | 42\| |
| | YVLDLWFSRRVRPQCRGEAYYFRFADDFVAGFQYRQEAEQFQTAL | [Thioflavicoccus |
| | GERLGQFKLRLAEEKTRCLAFGRFARSNAQKQGQKPGEFTFLGFT | mobilis |
| | H | 8321] |
| 8450 | GIDGVTVGEYAKALDENIADLVARLKAKQYKPQPVLRVYIPKPNG | >DS_UA.I7/FP5 |
| | EKRPLGIPAVEDKIVQMALKKILEAIFEQDFIDTSYGFRPNRSCH | 65147.1/ |
| | DALTELDRIIMNVPVNFVVDMDISKFFDTVDHKRLMECLRQRIVD | 1619711 . . . |
| | PTLLQLIGRFLKSGIMEEGKYSEMDQGTPQGGVLSPVLANVYLHY | 1621718/ |
| | VLDKWFENEVLPQLTGFAQLIRYADDFVVCFEKETEARAFGVALR | uncultured_ |
| | RRMGKFGLTISEEKSKIIEFGRCTCTRAKRYGRKCETFDFLGFTH | archaeon/ |
| | | BacterialE2 |
| 8451 | GVDGVTWRKYEENLDENTEDLVTRLIAKQYRPQPVKRAYIPKSNG | >DS_UA.I6/FP5 |
| | ERRPLGIPALEDKIVQLAIKKILEAIFEEDFCDVSYGFRPNRSCH | 65147.1/ |
| | DALDMVDMIIMTKPVSYVVDMDIAKFFDTVDHECLMECLKQRVVD | 2174432 . . . |
| | PSLLRIIARCLKSGVMEEGKYLETDKGTPQGGILSPILANIYLHY | 2176370/ |
| | ALDLWFEKEVKEQLKGFAQLIRYADDFIVCFQHDDEARAFGKTLR | uncultured_ |
| | ERLAKFGLTISEEKSRIIKFGRYACQQARKQSKKCATFDFLGFTL | archaeon/ |
| | | BacterialE2 |
| 8452 | GIDDVTKQEYSKELDNNIENLIVKLRNHSYKPQAVKRVYIPKGDG | >DS_Cl.be.I3/C |
| | KTRPLGIPSYEDKLVQMALNKILQSIYEAEFKDFSYGFRPKRNCH | P000721/ |
| | SAIKALNKVIENGRINYVVDADIKGFFNNVNHEWMIKFLEVRIGD | 3718265 . . . |
| | PNIISLVKKFLKAGLMDNGIIKTTEIGTPQGSIVSPTLANIYLHY | 3720149/ |
| | SLDLWFEKVIKRNFRGQSEITRYADDFVCCFQYESEARQFCRLLV | Clostridium |
| | SRLNKENLEVERTKSKLILFGRFAEEIRKSRGFKNAETFDFLGFT | beijerinckii/ |
| | H | BacterialE2 |
| 8453 | GVDKVTWEEYDVNVDENVETLIAKMKRFSYRPQPARRVYIPKANG | >DS_Ta.sp. I2/C |
| | KLRPLGIPCYEDKLVAAVMADILNEVYENIFLDTSYGFRPGRSCH | P000923.1/1286 |
| | DAIKELNRIIGRCKISYVLEADIKGFFDNVDQKQLMEFIAHDIDD | 631 . . . 1288551/ |
| | KNFSRYIVRFLKSGIMEEGKYHESDKGTAQGSPLSPILANIYLHY | Thermoanaerobacter |
| | TLDVWFAYLKRNGKFRGEAYIVRYADDFVMLFQYKSDADKMYEAL | sp./BacterialE |
| | PKRMAKFGLELAMDKTKILPFGRFAKQNSKDGKTETFDFLGFTF | |
| 8454 | GIDGETKASYGGNLEENLRNLLEQLKEGSYRPTPVRRKFIPKAGS | >DS_clostridiafi |
| | NKLRPLGIPVLEDKLVQNALVIILESIYEQDFLEDSYGFRPGRSQ | d\|115615774\|locus\| |
| | HDALKDLSRKIGTRKVGYIVDADIRGYFDHVDHEWLLKMLQERIS | VBIDehSp22 |
| | DSKILKLIKRFLKAGVMEEGKLSKTEEGVPQGGSLSPLLGNIYLH | 8777_1721\| |
| | YVLDLWENKIITKQCQGEAYLTRFADDTVACFQYQKDAERFYEAL | [Dehalobacter |
| | KKRLKKENLEIAEEKTRIIEFGRYAQRDVQRRGGRKPETFDFLGI | sp. CF] |
| | TH | |
| 8455 | GVDKVTKEEYETNLENNIDNLLIRMKTFKYRPQPVRRVYIDKSGS | >DS_Cl.be.12_( |
| | NKKRPLGIPAYEDKVVQLAINKILKSIYEQDFIDSSFGFRQNRSC | YP_001310744.1_) |
| | HDALKILNVYLSEKNVNYVVDADIKGFFDNVDHKWLMKFLEHRIA | Clostridium |
| | DKNLLRYIGRFLKTGIMENGKFYKVYEGTPQGGIISPTLANIYLH | beijerinckii |
| | YVLDIWENNFIKKKCKGQAYIVRYADDFVCCFQYEDEAKAFYEAL | NCIMB8052 |
| | KNRLDKFNLQVAEDKTKILYFGKNAYYDRKFKRAKLESYKDRTFD | |
| | FLGFTH | |
| 8456 | GIDGITKEQYGDNLEANIQSLLERLKRKAYRPQPVRRVYIPKPGS | >DS_Mo.th.I1/C |
| | DKKRPLGIPAYEDKIVQLAASKILNAIYEAEFLDMSFGFRPQRGC | P000232.1/ |
| | HDALKLLNYLIVARKVNYIVDADIKGFFDHVNHDWLMKFLGHRIA | 2324936 . . . |
| | DPNFLRFIRRFLKAGIMENGELRDATEGTPQGGIVSPILANIYLH | 2328581/ |
| | YVLDLWFEKAVRKHCRGEAYMVRYADDFICCFQYKHEAEAFYRAL | Moorella |
| | KARLAKFSLSVAEEKTKIIPFGRFATQWCKRMGQNKPDTFDFLGF | thermoacetica/ |
| | TH | BacterialE |
| 8457 | GVDQVTKQAYEENLEANIADLIGRMKRQAYKPQPVRRVYIPKEGS | >DS_Sy.wo.I1/N |
| | NKRRPLGIPSYEDKLVQKGLARILNTIYEQDFLDCSFGFRPGRGC | Z_AAJG01000003 |
| | HDALKVLNHIIERKKVNYIVDADIRGFFDHVDHEWMMKFLELRIA | /20007 . . . 22007 |
| | DPNLLRLIKRFLKAGVMEAGIVYDTPKGTPQGGIVSPILANIYLH | /Syntrophomonas |
| | YVLDLWFEKVVKKRCQGEAYLVRYADDFVCCFQNKSDAEWFYANL | wolfei/ |
| | RERLNKENLEVAEEKTRIIAFGRFADKESKKQGRKKPDTFDFLGF | Bacterial |
| | TH | E2 |
| 8458 | RRQYIPKKNGKLRPLGIPNIEDRIVQQAIVNVLSPKCEEHIFHKW | >DS_clostridiafi |
| | SCGYRPNLGIKRVMQIILWNIETGYNHIYDCDIKGFFDNIPHKKL | d\|54666312\|locus |
| | MKVLTKYIADGTVLDMIWAWLKAGYMEEGKFHPTDSGTPQGGVIS | \|VBIDesCar1680 |
| | PLLANLYLNELDWTLEEHGVRFVRYADDFLLFAKSKEDIERAAEV | 00_0691\| |
| | AKTTLDELGLEVSIEKTRFVDFDKDDFNFVGFSF | [Desulfotomaculum |
| | | carboxydivorans |
| | | CO1SRB] |
| 8459 | GINNNTMDEMSVGRIINLIQLINSGSYKPRPCRRTHIPKDARKPN | >DS_fid\|871144 |
| | GKKRPLGIPTGDDKLIQEVMRMLLEEIYEPVFSDWNYGFRPKRSC | 90\|locus\|VBIEsc |
| | HSALKEIRNSWKGTKWVCDVDIKGYFDNIDHDLLLKFLSKRIADN | Bla78014_3566\| |
| | KFLALLKKFLKAGYLDNWRYFGTHSGTPQGGIISPILANVFLHKL | [Shimwellia |
| | DEFMKNRISEFGKGGRRKPNPIYKRALQNRANRIKWIRQGFGASG | blattae |
| | MPADEQKIQKWRHEADELEKKLRTLSSVIMDDSEFKRMRYVRYAD | DSM4481 |
| | DFLIGVTGSKNEAKKIMKEVVDFVETELHLEISKEKSGIIDPKKG | = NBRC105725] |
| | FTFLGYEI | |
| 8460 | GVDGQTFDGFSPDKVRSIIERLANGTYRPQPARRVYIPKANGQKR | >DS_N.a.I1/AF0 |
| | PLGVPTTEDKLVQEVVRTILEQIYEPLFSRHSHGFRPKRSCHTAL | 79317/ |
| | ESIRAIWTGVKWLIDVDVVGFFDNIDHDVLVSLLEKRIADRRFVR | 43084 . . . 45661/ |
| | LIRGLLKAGYVEDWVFHKTYSGTPQGGVVSPMLANIYLHELDMFM | Novosphingobium |
| | QAKMAGFDKGKQRSPSPDARRIRNRLSYVRRTVDQLRAKGRGDDP | aromaticivorans/ |
| | RVTSFLEEIGRLKAERLAVPASDAFDPNYRRLRYCRYADDFIIGV | ML |
| | TGSKSEARQIMEEVRTYLSDHLKLAVSAEKSGIHKASDGARFLGY | |
| | EV | |
| 8461 | GIDGKTFEDFGPDRLAPLIASVATGAYKPKPVRRVFIPKGKGKRR | >DS_N.a.I2/AF0 |
| | PLGIPTRDDRLVQEVARQLLERIYEPVFSKASHGFRPGRSCHTAL | 79317/ |
| | EHVKAVWTGVKWLVDVDVAGFFENIDHDILLKLLRKRIDDERFID | 53812 . . . 56360/ |
| | LIRDMLKAGVMEGRAHTQTYSGTPQGGIVSPILANIYLHELDEFM | Novosphingobium |
| | AGRITAFEKGKTRATNPEYRRLAGRIAKRRERLKRLEASDNADQV | aromaticivorans/ |
| | TVKAILAEINTLSKQMRSLPSRDAMDAGFRRLRYCRYADDFLIGV | ML |
| | IGSKDDARGVFAEVRTFLTEVLALTVSEEKSGIRKASDGTKFLGY | |
| | EV | |
| 8462 | GTINNTVDGFSKNRVSKIINNIKNGNYKPTPVKRVYIDKKGSKKK | >DS_Bacillifid\|1 |
| | RPLGIPTFDDKLVQLVIKYILEAIYEPNFSENSHGFRKNRGCHTA | 8918679\|locus\|V |
| | LKQIKKSGSGTKWFIEGDIQGFFDNIDHHILINLLRKRINDETLI | BIBacCer120424_ |
| | GLIWKFLRAGYMEDWQFHKTFSGTPQGGILSPLLANIYLNELDIY | 5472\| |
| | MEKYAERFGKGQPKDREVDKRYQYLHLKIKRGRKKADLLREQGKL | [Bacillus |
| | NESQELIHQVNEWIKERGQRPYYNPMSDKFKSLKYVRYADDFIVM | cereusQ1] |
| | LIGSKDDANAIKSDIAQFLNEELKLTLSEEKTLITHSSKKAKFLG | |
| | YNV | |
| 8463 | GSTDETIDGMSMAKIHRIIADLRRETYRWTPVRRVYIPKATGKTR | >W_[Herpetosiphon_ |
| | PLGVPTWSDKLVQEVLRSILDAYYDPQMSDHSHGFRPNRGCHTAL | aurantiacus |
| | KAIQRCWTGTRWFIEGDIAQYFDTINHTTLLTILAKRIHDGRFLR | _DSM_785]1598 |
| | LIQTLLQAGYLHDWVYHPTLSGTPQGGVISPLLANIYLHEFDQFV | 98445 |
| | EHTLIPAYTKGQRRKVNPAYAQMEQRISKLRRQREYASVTPLLKE | |
| | LRTLPSRDVHDPDYRRLRYVRYADDFLLGFAGTKVEAEAIKQQIN | |
| | VWLYDHLQLKLSTQKTLITHASSDPAHFLGYDI | |
| 8464 | GVTEETIDGMSIQKIDMIIEQLRQETYYWRPARREYIPKKNGKHR | >DS_Bacillifid\| |
| | PLGIPVWSDKLLQEVIRMILEAYYEPQFSEHSHGFRPKRGCHTAL | 190354377\|locus\| |
| | QEIQTWQGTHWFIEGDISSYFDTIDHCVLITMLSKQIQDGRFIRL | VBIStrAng1666 |
| | IKNMLEAGYLDDWKFRKTISGTPQGGVISPLLANIYLHQFDKWVG | 16_0608\| |
| | EELIPQYTRGKKQKANSAYNRLSRKIKFYQDKGEYKKAHQIIVER | [Streptococcus |
| | RNIPSVDTYDTNYRRLRYVRYADDFILGFTGSKAEAKDIKKQIGD | anginosus |
| | FLNIKLHLELSQEKTLITHATEESAKFLGYEI | C238] |
| 8465 | GVDNRTIDGFKYEMIDTLIEKLKTEQYYPKPVRRTYIPKKNGKTR | >DS_clostridiafid\| |
| | PLGIPCFEDKLLQEVIRQLLESIYEPIFSDNSHGFRPDRSCHTAL | 47030643\|locus |
| | CQIKNTMRGANWVIEGDITGCFDNIDHTILLNILSQKIEDGRFIE | \|VBISynGly1059 |
| | LIRRFLKAGYLEFKQMHRSLSGCPQGGIISPILSNIYLNEFDRYM | 27_0075 |
| | DEIINKNTKGKKRRSNPEYQRLRGKRYTAIKKGNLEEIKRLTKEI | [Syntrophobotulus |
| | QSIPSLDPMDSNFTRVKYVRYADDFVIEVIGSKEMAESIKEDVAT | glycolicus |
| | FLKEKLNLELNQEKTLITNLGNEKANFLGYEF | DSM8271] |
| 8466 | GTDKETIDGFSMDWIENIISSLKDESYKPNPSRRVYIPKKDDKQR | >DS_Bacillifid\|1 |
| | PLGIPSIKDKIIQEVVKEILVSMYEPIFSKASHGFRPNKSCHSAL | 8911848\|locus\| |
| | NDIKMTFGGIKWWIEGDIKGFFDNIDHHVLIGILRKRIKDEKFIK | VBIBacCer120424 |
| | LIWKFLKAGYMEDWKFNKTFSGTPQGGIISPVLANIYLHELDAFM | _2093\|[Bacillus |
| | EKQIIKFDEGKRRRDNPVYKKYNTAIWYRKNKLKEKWNTLNDDER | cereus |
| | KELQSEISTLEKEREKHSAVDNMDASFKRLKYVRYADDFVVGVIG | Q1] |
| | SKEDSKRIKEEITEFLHTSLKLELSQEKTLITSNKNLIKFLGYEI | |
| 8467 | GVDQRSIDGFSMKEVEDLISVLKSKSYQPYPSRRTYIEKKNGKKR | >DS_Bacillifid\| |
| | PLGIPSFYDKLVQEVIRMILEAIYDSSFSSSSHGYRKGKGCHSAL | 54164737\|locus\| |
| | LEIKRTFTGSKWFIEGDIKGFFDNIEHHTLVTILKRRIKDEAFIE | VBIBacThu15523 |
| | LIWKFLRAGYLEEWKFHNTYSGAPQGGIISPIISYIYLNELDTYM | 2_5952\|[Bacillus |
| | KKYQDRFESGKKRQINKEYSNLQYKVRKIQEKIDTAYLNGEVTRI | thuringiensis |
| | TELKEQQKVLKGKLLQTPYNNPMDENYRRLKYVRYADDFLIGVIG | serovar chinensis |
| | SKEDAILIKNEIASFLKEEIKLELSMEKTLITNAFKKHAKFLGFE | CT43] |
| | I | |
| 8468 | GVDNQTISAMSLERINKIIDSLKDESYSPTPTKRVYIPKKNGKLR | >DS_Bacillifid\| |
| | PLGIPSIGDKLVQEVCRMLLNSIYDESFEDTSHGFRDNRSCHTAL | 19729760\|locus\|V |
| | RQIQNRFVRCKWFVEGDIKGFFDNIDHNIMIDILSKRIDDERFLR | BIStrPyo25933_ |
| | LIRKFLKSGYMEQNQYHNTYSGMPQGSIISPILSNIYLDKFDKYM | 1754\| |
| | QNYKESFDKGNKRKQNKEYKALYDRRKRLENKLSKTTNKTEIDDI | [Streptococcus |
| | KSEIEEINKRYFNIPCLNPMDENFKRIQYVRYADDFIIGIIGSKA | pyogenes |
| | DAEMVKQDIGQFIKSELNLELSDEKTLVTKSTDRAKFLGFDI | MGAS10750] |
| 8469 | GTDGKTIDGMGMARINALIEKMRNSSYQPNPARRTYIPKSNGKMR | >DS_clostridiafi |
| | PLGIPSFDDKLIQEVVRLILESIYEPTFSDHSHGFRMNKSCHTAL | d\|42835086\|locus |
| | KYVQKYFTGTKWFVEGDIKGCFDNVDHHVLIAILRKRIADEQFIG | \|VBICloCf15856 |
| | LLWKFLKAGYMEDWNYHNTYSGTPQGSIISPILANIYLNELDHFM | 9_1256\| |
| | AEYAEKENCGDRRRINPAFKKKLDVCRGKEERLKRNISKMSEEEK | [Clostridium |
| | EGLLAEISELRRSLRSMPYSDQMDEGYKRVFYIRYADDFLIGVIG | cf.saccharolyticum |
| | RKADAEQVKQDVGHFIRENLHLEMSEEKTLITHGHDFAKFLGYEV | K10] |
| 8470 | GTDGKTEDEMSIDRINKLIESIKDETYSPNPAKRIYIPKKNGKMR | >DS_Bacteroidet |
| | PLGIPSFEDKLVQEAVRMVLEAIYEGHFEWTSHGFRPNRSCHTAL | esfid\|46993147\|locus |
| | KSLQNNFNGAKWFIEGDIKGFFDNIDHDVLIEIMKGRIADDRFLR | \|VBIOdoSpl |
| | LIRKFLNAGYMEEWQFNKTYSGTPQGGIISPVLANIYLDKFDKYM | 147623_0215\| |
| | NEYANKFNKGTVRSRNKDICKLNSRVHYLKRRINEVEDVNVRTRM | [Odoribacter |
| | VEELHEKQKRILTMPSGNDMDRNFRRLRYLRYADDFLIGVIGTKN | splanchnicus |
| | ECETIKADITKFMQEKLRLEMSQEKTLITNAQDSAKFLGYEI | DSM220712] |
| 8471 | GTDGQTISGMSIKRIQSIIDKLRDESYQPHPAKRIYIPKKNGKQR | >DS_Ba.fr.I1/A |
| | PLGIPSFEDKLVQKVIQMILESIYEGSFEKCSHGFRPHRNCHTAM | Y515263/ |
| | ASIMEGFDGTRWFIEGDIKGFFDNIDHDIMITILSERIADERFLR | 38446 . . . |
| | LIRKFLNAGYLEKWKFHKTFSGTPQGGIISPILANIYLDQLDKYV | 40893/ |
| | VEYISQFNRGKMRKRNPEYKRIASRKDKRVKKLKTETDEQKRAAL | Bacteroides |
| | RSEIVELHREMQKHPATLDMDEDFRRMRYVRYADDFLIGIIGSKD | fragilis/ |
| | DCVNIKADIKRFLCEKLKLELSDEKTLITHGHDHAKFLGFEV | ML |
| 8472 | GVDELTIDGMSIARIDQLIDSLKDESYQPHPSRRTYIPKKNGKLR | >DS_Bacillifid\| |
| | PLGIPSFDDKLLQQVIKMILEAIYEGQFEPSSHGFRPNKSCHTAL | 19673908\|locus\|V |
| | TQIQKTYTGTKWFIEGDIKSFFDNINHDVMIHILRERITDERFLR | BIStrPne132160 |
| | LIRKFLNAGYVEDWKFYKTYSGTPQGGIISPILANIYLDKFDKYM | _1355\| |
| | TDYVKNFCQGKYRKRTPEYRQNEIALGKARRALECVSTENQRQEV | [Streptococcus |
| | IQRIRQLEKERVLIPHSDPMDSSFKRLTYTRYADDFICGVIGSKE | pneumoniae |
| | DAHRIKADIKDYLEAVLKLELSVEKTLITNARDKAKFLGYHL | ATCC700669] |
| 8473 | GADGKTIDGMSIDRVEQLIGSLKNETYQPNPSKRTYIPKKNGKKR | >DS_B.t.I2/ |
| | PLGIPSFDDKLVQEVVRMILEAIYEGSFEHTSHGFRPKRSCHTAL | AE015928/ |
| | IDIQKTFTAVKWFIEGDIKGFFDNINHDVLINILRERIADERFLR | 3241156 . . . |
| | LIRKFLNAGYVEDWVFHRTYSGTPQGGIISPILANIYLDKFDKYI | 3243662/ |
| | KEYINRFNKGVTRKGDARYKLYEQRRYRLAKKLKNEKDVKVRKQM | Bacteroides |
| | TAEIKRLREERNNYPARNEMDSSIKRLKYVRYADDFLIGITGNLE | thetaiotaomicron |
| | DCKTVKEDIKNYLNEALKLELSDEKTLITNAQKPAKFLGYDV | /ML |
| 8474 | GSDGKTIDGMSLKRIENLIDALKDESYQPKPARRTYIPKKNGNMR | >DS_Bacteroidet |
| | PLGIPSIDDKLVQEVLRMLLEAIYEGSFENTSHGFRPKRSCHTAL | esfid\|87116544\|locus |
| | IQVQKNFTAAKWFIEGDIEGFFDNINHDVLIGILKERIADDRFIR | \|VBIAliFin1 |
| | LMWKFLKAGYIEDWTFHRTYSGTPQGGIISPILANIYLDKLDKYM | 45170_0639 |
| | KEYACQFDRGDRRAMNLEYKRYSRKIWWLGTKLKQTKDKDTRKEL | [Alistipes |
| | IDAIKQHQKNRMHLPSVDEMDEGYRRIKYVRYADDFIIGVIGSKS | finegoldii |
| | DCEAIKEDIKNFLGEKLKLTLSEEKTLITHGNRKAKFLGYEI | DSM17242] |
| 8475 | GSDGRSIDEMSLARIETLIASLKDESYQPHPSRRVHIPKKNGKTR | >DS_Bacteroidet |
| | PLGIPAFEDKLVQEVVRMILEAIYEGHFETTSHGFRPKRSCHTAL | esfid\|87116554\|locus |
| | LHIQKTFSGAKWFIEGDIKGFFDNIDHDVLVGILRERISDDRFIR | VBIAliFin1 |
| | LIRKFLKAGYVEDWTFHNTYSGMPQGGIVSPILANIYLDKLDKYV | 45170_0644\| |
| | KEYIRHFDMGTKRRPGKESNDLANERKRTVRKLKKVKDGTEKAAL | [Alistipes |
| | VARLKAIEQERAAFPSGDEMDGSYRRLKYIRYADDFILGVIGSKE | finegoldii |
| | DALRIKEDIKSFLSESLALELSEEKTLITHTGKSAKFLGYEI | DSM17242] |
| 8476 | GTDGKTIVEIQKLPIEMVIKTIRNKLNYYQPKNVRRVEIPKDNGK | >DS_Bacillifid\|5 |
| | TRPLGIPSIWDRLIQQCVLQVLEPICEAKFHERNNGFRPYRSTQN | 8992730\|locus\|V |
| | AIAQCYKMAQIQNLHFVVDVDITGFFDNIDHSKLIRQLWGLGVQD | BIStrEqu204605 |
| | RKLIMIIKQMLKADILFKDIVITPETGTPQGGILSPLLANVVLNE | 0781\| |
| | LDWWVANQWEMFKIKEGSTGYEFTKVDNEGNILTIDRTQKWNKLR | [Streptococcus |
| | AKTGLKEMYITRYADDFKIFCRDYATAVKVMKATNLWLAENLHLQ | equi |
| | TSDEKSGITNLRKNYTTFLGIKF | sub sp. zooepidemicu |
| | | SATCC35246] |
| 8477 | GTDGTIIKDIGKLPAETVVKKVRYIVAGSPHGYRPKPVRRKEIPK | >DS_En.fm.I1/N |
| | PNGKTRPLGIPCMWDRLIQQCIKQVLEPICEAKFSENSYGFRPNR | Z_AAAK03000007/ |
| | SVENAIKATYNRLQISQLHYVIEFDIKGFFDNVNHSKLIKQIWAM | 10877 . . . 13634 |
| | GIRDKHLIFILKRILKAPIKMTNGTITYPEKGTPQGGIISPLLAN | /Enterococcus |
| | IVLNELDHWVESQWQENPVTKNYVVHINKSGSPCKSNAYKEMKKT | faecium/ |
| | KLKEMYMVRYADDFRVFCRYKESAEKAKIAITQWIEQRLKLEVSQ | BacterialB |
| | EKTRIVNVRKRYSDFLGFKI | |
| 8478 | GTDKLKISDIGKLTADEVTARVRRIVKGGKNGYTPRSVRRKEIPK | >DS_clostridiafi |
| | PNGSTRPLGIPCIWDRLVQQCIKQVMEPICEARFSNNSYGFRPNR | d\|42996817\|locus |
| | SVENAIAAIYRLMQRSGLYYVVEFDIKGFFDNVDHSKLIKQLWSL | \|VBIEubSir1356 |
| | NIRDKELLYVIRRILKAPILMPDGHIEHPAKGTPQGGIISPLLAN | 46_1742\| |
| | VVLNELDHWIESQWQCNPVTENYSYRENATGCPIQSHAYRAMRNT | [Eubacterium |
| | RLKEMYIVRYADDFRILCRTKEQADRTLIAVTHWLKERLRLDVSP | siraeum |
| | EKTRVVDTRRSYSEFLGFKI | 70/3] |
| 8479 | ACDNVNIKNIEGMEQSYFLNEVKRRFQNYQPQKVRRKEISKPNGQ | >DS_B.a.I2/AE0 |
| | TRPLGIPAMWDRIIQQCILQVMEPICEAHFSNRSYGFRPNRSAEH | 11190/ |
| | ALADASVRVNKQNLTYVVDVDIKGFFDEVNHVKLMRQLWTLGIRD | 30945 . . . 33835/ |
| | KQLLVIIRKILKAPVQMPDGTTMFPTKGTPQGGILSPILANVNLN | Bacillus_ |
| | EFDWWISRQWETFKAKKVKPRCMRGIWCNDVVTTQLTKTSKMKPM | extractionanthracis/ |
| | YIVRYADDFKIFTNTRSNAEKIFKATQMWLEERLKLSISAEKSKV | BacterialB |
| | TNLTKQQSEFLGFTL | |
| 8480 | GIDGKTIKDIEKLTTERYLDIVKKRFKFYKPRKVKRTEIPKPNGK | >DS_En.fm.I3/F |
| | TRPLGIPSIWDRVAQQCILQVLEPICEAKFNPHSHGFRPNRSAEH | N424376.1/ |
| | AIADCAKKMNIIKMGYCVDIDIQGFFDEVWHSKLMRQMWTMGIRD | 17411 . . . 20180/ |
| | KELLTIIRKMLKAPVVLPNGTIQFPEKGTPQGGILSPLLANINLS | Enterococcus |
| | EFDWWVSEQWETRHMSEIKTQYNANGTEHMGNHHRKMRSHTKLKE | faecium/ |
| | FYIVRYADDFKLFCHNRKTAELLYHASIQWLEQRLHLPVSIEKSK | BacterialB |
| | ITNLRKESSEFLGFNL | |
| 8481 | GVDDITIKDIENLEQTIFVEMVRKRFSNYSPRKVRRVEIPKPNGK | >DS_Bacillifid\|2 |
| | TRPLGIPSIWDRIAQQCILQVIEPICEAKFNKHSYGFRPNRSTEH | 02064373\|locus\| |
| | AIADMLFRINQQKLHYVVDVDLQGFFDEINHKKLMNQVWTLGIHD | VBICarSp26422 |
| | KQLLVIIRKMLSAPIVLKNGSIMHPVKGTPQGGILSPLLANISLN | 3_1846 |
| | EFDWWISNQWETFETRKKYAAAVMGNGTKNRGLTYRMLRKNSKLK | [Carnobacterium |
| | EIYIVRYADDFKLITSNRRDAEKIFIASQMWLKERLGLPISKEKS | sp. WN1359] |
| | KITNLRKEESEFLGFTI | |
| 8482 | GIDGVTIKDVEKLSQEDFIKIVQKRFSNYTPRKVRRVEIPKPNGK | >DS_Bacillifid\|1 |
| | TRPLGIPSMWDRIAQQCIKQVLEPICEAKFNKHSHGFRPNRSPET | 8859935\|locus\|V |
| | AMADATLRVNRSHMQYVVNVDIQGFFDEVNHKKLMRQLWTMGIRD | BIBacCer118379 |
| | KQLLVIIRKMLKAPIVLPNGEMQYPNKGTPQGGILSPLLANINLN | _5432\| |
| | EFDWWITNQWEDRLLKELSLTIKKGGHVDKYPHYSKMRKTTALKE | [Bacillus cereus |
| | MYIVRYADDFKIFTATKSNAQKIFKACEMWLQERLKLPISKEKSK | ATCC10987] |
| | ITNLRKESSEFLGFEI | |
| 8483 | GVDGITISDIERLNENDFVEIIRANLSNYRPGPVRRVYIPKKNGK | >DS_Bacillifid\|1 |
| | KRPLGIPNLYDRIIQQTIKQVIEPIVEAKFFKHSYGFRPLRSVEQ | 90447919\|locus\| |
| | AMGRMHSVINNVQLHYVVDVDIKGFFDNVNHNLLRHQIWNMGIRD | VBIEntSp29956 |
| | TKLIAIISKILRAEIVGEGTPVKGTPQGGVLSPLLANIVLNDLDQ | 9_0686\|[Enterococcus |
| | WIASQWENFPSKHRYSRGKLHRALKGTTLKEGYLVRYADDFKLLT | sp. HSIEG1] |
| | RSYSMAKRWYTAIRGYIEKHLKLEISPEKSGITNLRKKRTEFLGF | |
| | EI | |
| 8484 | GTDGKTIDDMKELSENDLVNEVRSKLQNYHPKKVRREWIEKENGK | >DS_Bacillifid\|8 |
| | WRPLGIPCILDRVIQQCFKQVLEPIVESQFFKHSYGFRPLRSAHH | 7137209\|locus\|V |
| | AMARIQFLINHSQLHYVVDVDIKSFFDNVNHRLLKKQLWNIGIQD | BIHalHal165146 |
| | RKVLACISKMITSEIDGEGVPDKGSPQGGILSPLLSNVVLNDLDQ | 0228\| |
| | WVADQWEVFPLTKSYSSDDARRRARKQTNLKQGYLVRYADDFKIL | [Halobacillus |
| | CRDGKTAQRWYHAVRLYLKERLKLDISPEKSQIVNLRKRESEFLG | halophilus |
| | FTI | DSM2266] |
| 8485 | GTDSFTIDNYKEMNQAEFIHLILSQLENYKSKSIKRVMIPKPNGE | >DS_Bacillifid\|4 |
| | KRPLGIPCMIDRIIQQMFKQVLEPICEAKFYEHSYGFRPLRSAKH | 7119490\|locus\|V |
| | ALGRIMYLINISKMHYAVDIDIKGFFDNVNHRLLIKQLWNIGICD | BIEntFae176554 |
| | KRVLAILSKSLKSPIQGEGISSKGTIQGGIISPLLSNVVLNDLDH | 2204\| |
| | WVSKQWHTFETKYPYTKGYNKFRALRDTNLKQGYIVRYADDFKIM | [Enterococcus |
| | TNDYPSALKWFHAVKLYLKDRLKLDISNEKSKIVNLRKRKSEFLG | faecalis 62] |
| | FTI | |
| 8486 | GIDSFAIDQYKSMDKAEFLNLVRNRLNQYKPKAVKRVFIPKPNGD | >DS_Bacillifid\|1 |
| | KRPLGIPTMFDRLIQQMIKQILEPICEAKFYEHSYGFRPLRGARH | 01938694\|locus\| |
| | AISRVMYLISRNTFHYAVEIDIKGFFDNVNHTLLLKQLWNMGIKD | VBIBac Thu2420 |
| | KRVLKLIYLILKAPIKGVGIPRKGTPQGGILSPLLSNVVLNDLDQ | 10_5758\| |
| | WIARQWHHFQSDYDYTEPGNRSRALKRTKLKQGYIVRYADDFKIM | [Bacillus |
| | AKDFRTAQKWFMATKLYLKERLKLDISPGKSRIINLRKNKSEFLG | thuringiensis |
| | YSL | MC28] |
| 8487 | GTNGHTIKHLNKIDADKLIRLTQKRLENYMPHAVRRLFISKPNGK | >DS_Bacillifid\| |
| | MRPLGIPTIEDRLIQQMFQQVLEPIVEGKFHPQSYGFRPKRGTHD | 18820991\|locus\|V |
| | ALARCYHMVNHSHQHFVVDIDIKGFFDNVNHKKLMRQLWTIGIRD | BIBacCer84800_ |
| | KKVLSIIKKMLKAEVTGEGIPVKGTPQGGILSPLLANVVLNELDW | 3811\| |
| | WVSNQWETKPTRVPYKLKRNKTDALKKTRLKPMYLVRYADDFKIF | [Bacillus |
| | TNSYDNARKIKIAVEKWLKERLGLEISEEKSKITNLRKNGTDFLG | cereus |
| | IRF | 03BB102] |
| 8488 | GTDGLTIKDIAGMTNQEVITMVKRRLKNFTPQSVRRVEILKDNGQ | >DS_clostridiafid |
| | NRPLGIPTMSDRLIQACIYQILEPICEARFHNHSYGFRPTRRTEH | \|115616442\|locus\| |
| | ALATMHRMINIQHLHFVVDVDIKGFFDNVDHGKLLKQMWTMGIQD | VBIDehSp22 |
| | KNLLCIISAMLKAEIEGIGIPNKGVPQGGLCSPLFSNVVLNELDW | 8777_1269\| |
| | WISDQWESYETSYPYKRNEGKIRAIRRGSKLKECYIIRYCDDFKI | [Dehalobacter |
| | MCPTRDVAERMFVAVKLWLKERLNLEISSEKSKITNLRKKSSEFL | sp. CF] |
| | GFKI | |
| 8489 | GTNKRTIIDVGEENPYQLVQYVQNRENNFQPHSIRRVEIPKPNGK | >DS_C.d.I1/X98 |
| | TRPLGIPTIEDRLVQQCIKQILEPILEAKFHKHSYGFRPERSSHH | 606/ |
| | AIAIFQQWTFKGFHYVVDIDIKGFFDNVNHGKLVKQLWTMKIRDK | 13 . . . 2658/ |
| | TFISILSRMLKAEVKGIGKSTKGTPQGGILSPLLANVVLNELDWW | Clostridium |
| | IDSQWDGFPTKRKYSSLLSKTQSIRKYSNLKEIKIVRYADDFKIM | difficile/ |
| | CKDYHTAQKIFLATKQWLKVRLDLDISPEKSKVTNLRKNYSDFLG | BacterialB |
| | FKL | |
| 8490 | GVDNKNIDDLKSIPDTDFISIVQTKLSEYKPQPVKRVEIPKPNGK | >DS_Bacillifid\|2 |
| | TRPLGIPTIWDRIVQQCLLQVLEPIMEAKFHDKNYGFRPNRSAHH | 02104716\|locus\| |
| | AFAQAVRMAQVSKLTFVVDIDIEGFFDNVNHSKLIKQLWSLGVRD | VBIEntMun2812 |
| | KWLLGVIRAMLKAPIIHKDGHIEHPKKGTPQGGILSPLLANVVLN | 67_0501\| |
| | ELDWWISSQWETHPTRHNYDWYHAEKEYWNKGNKYRALRGTSLKE | [Enterococcus |
| | IYIVRYADDFKIFCRKRSDADKIFLATKLWLKERLKLDISQEKSK | mundtii |
| | VVNLKKQKSEFLGFTL | QU25] |
| 8491 | GTDTLNIKDIEKLSVEKLVEMMQRKLAWYQPKPVKRVEIPKPNGK | >DS_clostridiafid\| |
| | TRPLGIPTIVDRLVQQCILQVLEPICEAKFYERSNGFRPNRSAEH | 19436501\|locus |
| | AMAQCYRMVQKQNLYFVVDVDIKGFFDNVNHSKLIRQMWAMGIRD | \|VBICIoCel5778 |
| | KQLICIIKQMLKAPVVMPDGETLYPTKGTPQGGILSPLLANIVLN | 3_2839\| |
| | ELDWWISSQWEDMLTHREYYVSVNNNGSLNKSGVFRTLRRSALKE | [Clostridium |
| | MYIVRYADDFKIFCRKRSDANKIFVAVKKWLKDRLKLEISEEKSK | cellulolyticum |
| | VVNLKKHYSEFLGFQF | H10] |
| 8492 | GVDGRTIKHLSRLNEEEYISLIQKQFHWYKPRPVKRVEILKPNGK | >DS_Bacillifid\|1 |
| | IRPLGIPTIVDRIVQQCILQILEPICEAKFHDSSYGFRPNRSTEH | 90355818\|locus\| |
| | AIAECARLMQIQHLHYVVDIDIQGFFDNVYHAKLIRQLWNLGIQD | VBIStrAng1666 |
| | KKLLCIIKEMLKADIVMPDKEVITPTKGTPQGGILSPLLSNVVLN | 16_1315\| |
| | ELDWWVSSQWLTMPTHYPYKQRTNSQGTEIKSHTYRALRTSNLKE | [Streptococcus |
| | IYIVRYADDFKIFCRNYYDAKRTYQAVTKWLQDRLKLNVSEEKSK | anginosus |
| | ITNLKQRYSEFLGFKL | C238] |
| 8493 | GVDKRTIADLAKLSEEEYVRLIRKQFSNYHPGPVRRVEIPKPNGK | >DS_E.f.I3/AE0 |
| | TRPLGIPTIVDRIVQQCILQVMEPICEAKFSENSNGFRPNRSAET | 16830/ |
| | AIAQCMRLIQVQHLYHVVDLDIKGFFDNISHTKLIRQIWALGIRD | 2249712 . . . |
| | KKLLCIIKEMLKAPVVLPNGEKTYPARGTPQGGILSPLLANIVLN | 2252481/ |
| | ELDWWIASQWEEMPTKTKFKTRSNAQGTEIKSHAYRALRRSRLKE | Enterococcus |
| | MHAVRYADDFKIFCATHEDAVRAYKATELWLKDRLGLEISPDKSK | faecalis/ |
| | VVNLKRQYSDFLGFKL | BacterialB |
| 8494 | GTNNKTIKDLEEKSTEELVEYVRNRLEYYVPQSVRRVYIPKPDGR | >DS_clostridiafid\| |
| | KRPLGIPTIKDRLIQQCIKQVLEPICEAKFHNHSYGFRPNRSTKH | 115343359\|locus\| |
| | AIARIMYLINFSKLHYTVDIDIKSFFDNVDHNKLKKQLWSMGIRD | VBIHalHal14 |
| | KKLISILGNMLEAKIEGEGVPEKGTPQGGIISPLLSNIVLNEMDW | 9681_0148\| |
| | WISNQWETFKTDYKYNRKGDKITAIKKTNLKEIYIIRYADDFKIM | [Halo |
| | CRDFETASKIKIATIKWLKERLNLEVSEKKTSITNLKKNHTEFLG | bacteroides |
| | IKL | halobios |
| | | DSM5150] |
| 8495 | GTNHKTINDIAGESEDEIIEYVRKRLNKFYPHSVKRIYIPKNNGD | >DS_clostridiafi |
| | KRPLGIPTIEDRLIQRSILQVLEPICEAKFHPHSYGFRPNRSTEH | d\|19408375\|locus |
| | AIARAMTLINMNKLHYVVDVDIKGFFDNVNHGKLLKQLWTLGIKD | \|VBICloBot1990 |
| | KKLIKIISLMLKAQIKDGSMITNPVKGTPQGGIISPLLANVVLNE | 8_0265\| |
| | LDWWISSQWETFETKHNYSKLRTFKNGTTTIDKSHKYRALRNGKL | [Clostridium |
| | KEIYIVRYADDFKVFCKNPKDAEKIFIAIKLWLKERLDLETSPEK | botulinum |
| | SKVTNLRKHPTEFLGFEL | Ba4str.657] |
| 8496 | GSNDTTILEIAEQNLTTFVAKVQKALENYNPKPIRRVYIPKRNGD | >DS_Bacillifid\|3 |
| | KRPLGIPTMEDRIVQQCIKQILEPICEAKFYNHSYGFRPNRNAKH | 8137486\|locus\|V |
| | AIVRAMSLMNISKFHYVVDIDIKGFFDNVNHGKLLKQIWSLGIRD | BIBacThu148000_ |
| | KSLLSIISKILKTEIENVGKMEKGTPQGGIISPLLSNIVLNELDW | 5492\|[Bacillus |
| | WISSQWETMITRHNYESIDKRNNTIIRSHKYTALRRTSNLKEMFL | thuringiensis |
| | VRYADDFKIFCKDENSAQKTLIAVKKWLKNRLGLEVNNEKSKVTN | BMB171] |
| | LRRNYTEFLGFKL | |
| 8497 | GTDGITIEQYKIEDVETFVDEIRATLKNYKPQTVRRVEIPKPNGK | >DS_Bacillifid\|2 |
| | TRPLGIPTMRDRLIQQMFKQILEPICEARFYNHSYGFRPNRSTHH | 2412306\|locus\|V |
| | AMGRCQFLANIALNQHVVDIDIQGFFDNVSHSKLLKQMYSIGICD | BILysSph89750 |
| | KRVLSVVSKMLKAPIKGIGIPTKGTPQGGILSPLLSNIVLNDLDW | _0101\|Mobileele |
| | WISNQWENMKTKFNYKERKNKVLMIKRTTTLKEMYIVRYADDFKI | mentprotein |
| | FTKSHKNAIKLYHAVKGYLKNHLNLDISNEKSKITNLRKRASEFL | [Lysinibacillus |
| | GFSL | sphaericus |
| | | C341] |
| 8498 | GTDGITIDDYKLANIEIFVSYIRSVLSNYKPQKVRRVYIPKSNGK | >DS_Bacillifid\|2 |
| | KRPLGIPTMRDRIIQQMFLQILEPICEAQFYNHSYGFRPNRSTKH | 02109853\|locus\| |
| | AMARCKFLTRKNFHYVVDIDIKGFFDNVNHNKLIKQLYTIGIKDK | VBIEntMun2812 |
| | RVLAILAKMLKATIEGEGIPKKGTPQGGILSPLLSNVVLNELDWW | 67_2992 |
| | IANQWEFLKTKENYHPAARLKSLKRKTTLKEMFIVRYADDFKIFT | [Enterococcus |
| | KDHQSAIRIYHGVKGYLSNHLSLDISPEKSKITNLRKRDSEFLGF | mundtii |
| | SL | QU25] |
| 8499 | GVNTNTIMDIGEENPDELAIYVRERLINYKPQPVRRVEIPKPNGK | >DS_clostridiafi |
| | MRPLGIPTIEDRIIQQCIKQVLEPICEAKFHKDSYGFRPNRSTHH | d\|19462591\|locus |
| | AIARTYSLANINKLTYVVDIDIKGFFDNVNHSKLLKQMWTMGIQD | \|VBICloKlu1115 |
| | KNLLCVISKMLKAEIKGVGIPNKGTPQGGILSPLLSNIVLNELDW | 49_0642\| |
| | WISNQWQTLKSKFPYKREIFKYQALKRSKLKEVYIVRYADDFKLF | [Clostridium |
| | CRSYNNAKKIFKAVTMWLKERLGLEINEEKSSIVNLKQKYSEFLG | kluyveri |
| | FKF | DSM555] |
| 8500 | GTDGMTIDDIKQLSNAEIVATVRESLSNYRPKSVRRVFIPKAGSD | >DS_Bacillifid\|6 |
| | KMRPLGIPCIWDRLVQQCILQVLEPICEPKFHNHSYGFRANRSAH | 7659680\|locus\|V |
| | HAVSRVTTLINLSKYHYCVDVDIKGFFDNVNHGKLLKQIWTLGIR | BIEntFae233823 |
| | DKRLICIISKMLKAEIDGEGVPEKGTPQGGLLSPLLSLIVLNELD | 1913\|[Enterococcus |
| | WWVSSQWETFQPKNRSKNGWLQYAKKYTKLKSGFIVRYADDFKIM | faecium |
| | CSTYGEAQRFYHSTVDFLNKRLKLEISPEKSKVVNLKKNSSDFLG | Aus0004] |
| | FKI | |
| 8501 | GVDNLTIKDIWHLNDTKIIHEVRKRLNNYQPQAVKRVLIPKEGSD | >DS_Bacillifid\|1 |
| | KKRPLGIPTIWDRLVQQSILQVLEPICEAKFHNHSYGFRPNRSTH | 8825078\|locus\|V |
| | HALSRVVSLINIGHQHYCVDIDIKGFFDNVCHKKLLRQMWTLGIR | BIBacCer120511 |
| | DKSLLCVISKILKSEIEGEGIPNKGTPQGGIISPLLSNIVLNELD | 0128\|[Bacillus |
| | WWISSQWETYKPHRISTRHLGFRQYARKYTNLKCGYVVRYADDFK | cereus |
| | IMCRTYDEAQRFYHATVDFLKSRLGLEINPKKSKVVNLKKNSSVF | AH187] |
| | LGFKI | |
| 8502 | GVDGLTIKDVRQLNDFQVINQVRKRLMNYRPSPVRRVYIPKEGSD | >DS_Bacillifid\|2 |
| | KKRPLGIPTIWDRLVQQCILQVLEPICEAKFHNHNYGFRPNRSTH | 01989473\|locus\| |
| | HALSRMVSLINVGKHHYCVDIDIKGFFDNVQHGKLLKQMWAIGIR | VBIBacThu9392 |
| | DKRLLSIISNLLKAEIIGEGIPSKGTPQGGILSPLLSNIVLNELD | 6_0768\|[Bacillus |
| | WWISNQWETYKPHRFKDGPNGFTTYARKYTNLKGGYIVRYADDFK | thuringiensis |
| | IMCRTYEEAQRFYHATVDFLKARLGLEINPEKSKVVHLKKNSSDF | YBT1518] |
| | LGFKI | |
| 8503 | GTDGMTINDIKMLSTDEVIEKVKMMFGWYEPQSVRRVFIPKPNGN | >DS_Bacillifid\|1 |
| | RRPLGIPTIWDRLFQQCVLQILEPICEAKFHNHSYGFRPNRSTHH | 01939315\|locus\| |
| | ALARMKSLVNRKGNGFHYCVDIDIKGFFDNVHHGKLLKQLWTIGI | VBIBacThu2420 |
| | RDKKLLSIISRLLKAEIVNEGVPQKGTPQGGILSPLLSNIVLNEL | 10_6066\| |
| | DWWVSNQWETIKTSHPYKGNSDKYRALKKSKLKECFLIRYADDAK | [Bacillus |
| | ILCRDYVTALKMFEATKDFLRTRLHLDISLEKSKIINLRKKASHF | thuringiensis |
| | LGFTV | MC28] |
| 8504 | GTDGSTIKDINNIDIDEVITKIKTMFDFYTPKSIRRVEIPKANGK | >DS_clostridiafi |
| | TRPLGIPTIWDRLFQQCILQVLEPICEAKFHKHSYGFRPNRSTHH | d\|54454697\|locus |
| | AITRSVYLINITKLYHCVDVDIKGFFDNVNHGKLLKQLWALGVKD | \|VBICloBot1788 |
| | KKLLKIISVMLKAPIEGIGIPTKGVPQGGILSPLLSNIVLNELDW | 72_0058 |
| | WVSNQWETFKTDKDYTKYRTSKTGKIVVDHSIRNKMLKKSKLKEI | [Clostridium |
| | YIVRYADDFKIFCRTRSQAKAIDIAVGDMLKNRLGLECSAEKSKV | botulinum |
| | LNLKKSYSEFLGFKM | BKT015925] |
| 8505 | GDDGLTIEDINRLSVSEVVSTIQRMFEYYTPQAVRRVFIPKANGK | >DS_B.me.I1/A |
| | TRPLGIPTIWDRLFQQCILQVLEPICEAKFYKHSYGFRPNRNTHH | B022308/ |
| | AKARFETLINRACLYHCVDVDIKGFFDNVNHAKLIKQLWSLGIRD | 3853 . . . |
| | KALLSIISRLLKAEIIGEGFPKKGTPQGGILSPLLSNIVLNELDW | 6569/Bacillus |
| | WVSNQWESFETHKLYKSNLGRYNALKQSNLKHCYIVRYADDFKIL | megaterium/ |
| | CRTRSQAIKMYYAVNDFLHTRLRLEISEQKSKVVNLKKNSSEFLG | BacterialB |
| | FRS | |
| 8506 | GTDGKTISDILTLNYDEAINFVKRCFKKYTPNPIRRVHIPKPGKK | >DS_G.k.I1/BA |
| | EKRPLGILTIADRIIQECVRMVIEPILEAQFFQHSYGFRPYRDAK | 000043/1312755 |
| | QAIERCVFICNRIGYNWVIEGDIKGFFDNVNHTILIKQLWHMGIR | . . . 1315536/ |
| | DRRMLMIIKAMLKAGVIKETKINEMGTPQGGIISPLLANVYLHKL | Geobacillus |
| | DQWITREWEEKKMRNGTTIRTAKYKSLRDHSTITKPEFYVRYADD | kaustophilus/ |
| | WVLFTNSRGNAEKWKYRIKKYLKENLKLELSDDKTLITNIKKKPM | BacterialB |
| | KFLGFKI | |
| 8507 | GTDGETIDDILQDGYESVISRVRKCFLAYNPKLLRRVHIDKQVSK | >DS_Bacillifid\|3 |
| | DKRPLGIPAIIDRIIQECIRMIIEPILEAQFFSHSYGFRPYRSAE | 1950695\|locus\|V |
| | HALSKVTNTAYDTNYCWVVEGDIKKFFDNVNHTILIKKLYSMGIR | BIBacPse80461 |
| | DRRVLMIIKAMLQCGVLGEAEQTTVGTPQGGIISPLLANAYLDSL | 4012\|[Bacillus |
| | DHWITREWENKETKHEYSRLDGKYRALKNASNLKPAHFVRYADDW | pseudofirmus |
| | VLITNSKANAIKWKQRIAKHLKEQLKLELSEEKTLITNIKKKAIK | OF4] |
| | FVGFHF | |
| 8508 | GVDGKTIQDYLRLSEEKLIELIRGRLTNFKAHLIKRVFIPKANGG | >DS_B.c.I5/AE0 |
| | QRPLGIPTIEDRIIQQMMKQVLEPVLEAQFFKYSFGFRPERTTYH | 17195/ |
| | ALERVKVLVHNTGYHWIVEGDIRQFFDKVNHRILIKKLWSMGIKD | 84166 . . . 86938/ |
| | RRILCLITEFLKAGIFKNIIRNDNGTPQGGILSPLLANVYLHSFD | Bacillus |
| | KWVAKQFEEFTTRHEYSKHDHKLRGLKSSNLKPGYLIRYADDWVL | cereus/ |
| | VTNNKSHAYRWKTVIKNFLQKELKLELSEEKTRITNIRHKPIEFL | BacterialB |
| | GFKY | |
| 8509 | GVDSLTINDILQADEEKVIHLITNTIRDYTPSMVRRVWIPKAGKK | >DS_Bacillifid\|2 |
| | ELRPLGIPTILDRIIQQCVKQVIEPICEAQFFPYSFGFRPYRDGH | 02001215\|locus\| |
| | MAIERVGSLIHKTKYHWIVEGDIRKFFDKVNHNILLKNCFKIGIQ | VBIBac Thu9392 |
| | DKRVLMLIKAMLKAGVMHENTKTTLGTPQGGIISPILANIYLHDF | 6_6557\|[Bacillus |
| | DMWVYNQWQNKKTRKNYANKHSRTTTLKRTTKLKQGYLIRYADDW | thuringiensis |
| | VIVTNSKTNAIKWKKAVSHYLKDKLKLELSEEKTKITNVRKKNIE | YBT1518] |
| | FLGFKL | |
| 8510 | GIDQKIVDDYLLMPTEKVFGMIKAKLNDYKPIPVRRCNKPKGNAK | >DS_Bacillifid\|4 |
| | SSKRKGNSPNEEGETRPLGISAVTDRIIQEMLRIVLEPIFEAQFY | 5223831\|locus\|V |
| | PHSYGFRPYRSTEHALAWMLKIINGSKLYWVVKGDIESYFDHINH | BIGeoSp94955 |
| | KKLLNIMWNMGVRDKRVLCIVKKMLKAGQVIQGKFYPTAKGIPQG | 1285\| |
| | GIISPLLANVYLNSFDWMVGQEYEYHPNNANYREKKNALAALRNK | [Geobacillus |
| | GHHPVFYIRYADDWVILTDTKEYAEKIREQCKQYLACELHLTLSD | sp. Y412MC52] |
| | EKTFIADIREQRVKFLGFCI | |
| 8511 | GIDNKTIDYYLHLPYEDLVSQVQTCIEDYNPEPVRRKYIPKENSD | >DS_Bacillifid\|3 |
| | KLRPLGIPTMIDRIIQEITRLVIEPIAEAKFYKFSYGFRPMRSAE | 1950623\|locus\|V |
| | HAMAEILEKARKSKTYWVIEGDIKGYFDNINHNKLITMLWKIGIK | BIBacPse80461 |
| | DKRVLSIIKKMLKSGIVEEDGEIYPSDLGSPQGGIISPLLANIYL | 3976\|[Bacillus |
| | NFFDWMIAEEFDQHHYINNYERRDKGLRAIRRDHKPVYSIRYADD | pseudofirmus |
| | WVVLCSSKKQADTLLIKIRKYLKHQLSLELSEEKTKITNLVEEKA | OF4] |
| | SFLGFEF | |
| 8512 | GIDKKDVNYYLQMEAKQLIKLIRQHIDNYKPNPVRREYINKGNGK | >DS_Bacillifid\|1 |
| | KRPLGIPTMIDRIIQEIARIVLEPIAEAKFFNHSYGFRPYRSCHY | 8919101\|locus\|V |
| | AIGRVLNTISRSKTYIAIEGDIKSFFDHINHNKLVEMMWNMGIKD | BIBacCer120424 |
| | KRFLIIIKKMLRAGVLEDKVILPTEIGTPQGGIISPLLANIYLNN | _5683\|[Bacillus |
| | FDWMVAKEFEEHRARYTVKHAFRSGLTKVGRRHKKCFLIRYADDW | cereus |
| | IILCEDTVQARILLTKIDKYYKHILKLELSKEKTFITDLREKPAR | Q1] |
| | FLGFDI | |
| 8513 | GVDGTTINDYLQMDRKQLINLIQSQIDNYNPSTVRRTYIPKGNTG | >DS_Bacillifid\|9 |
| | KLRPLGIPVIVDRIIQEIARMAIEPYCEAKFYPHSYGFRPYRSSE | 6574781\|locus\| |
| | HAIARIVQNINSKAYIAIEGDIKGYFDNINHNKLLAILWEMGIKD | VBIBacCer255427 |
| | KQFLFLIKKMLKSKILDNGNIISSDKGTPQGGIISPLLANVYLNN | _4629\| |
| | FDRMVSDLWESHSAVTTYAATRNGKTVEEKNYQFLRKKSVAKHYK | [Bacillus |
| | TNLVRYADDWIILTETKEYAEKLLTKLRKYMKHQLSLELSEEKTV | cereus FRI35] |
| | ITDSREEPLHFLGFRI | |
| 8514 | GIDGVTIEQMDDYLHQNWRETKKLIKERSYKPQPVLRVEIPKPNG | >DS_S.ag.I1/AJ |
| | GVRNLGIPTAMDRMIQQAIVQVLSPLCEKHFSEYSYGFRPNRSCE | 292930/ |
| | TAIVQLLEYLNDGYEWIVDIDLEKFFDTVPQDRLMSLVHNIIQDG | 182 . . . 203 |
| | DTESLIRKYFHSGVVINGQRHKTLVGTPQGGNLSPLLSNIMLNEL | 8/Streptococcus |
| | DKGLEKRGLRFVRYADDCVITVGSEAAAKRVMHSVSSYIEKRLGL | agalactiae/ |
| | KVNMTKTKIVRPNKLKYLGFGF | BacterialC |
| 8515 | GIDNMSIEEFNDFAKLHWLGIKQQLLNGSYQPLPVKRVMIPKPDG | >DS_E.c.I7/AY7 |
| | GERMLGIPAVIDRVIQQAIAQVISPYFEPQFSPHSYGYRPHKRAS | 85243/ |
| | QAVNHVQSCVKQGYKTAVDIDLSKFFDEVDHDMLMNRVSRKIKDK | 414 . . . 2383/ |
| | ALMRLLGKYLRAGIAERETGLWFESTKGVPQGGPLSPLLSNILLD | Escherichia |
| | ELDKKLTYKHLKFARYADDIIILVKTKSEGLIIQREITAFITKRL | coli/ |
| | KLKVNESKSRVGPVSGSKFLGFTF | BacterialC |
| 8516 | GIDDMTVNDLLPYLRENKTELIASLREGKYKPAPVKRVEIPKPNG | >DS_La.re.I1/A |
| | GVRKLGIPTVVDRMVQQAVAQILTPIFERVFSDNSFGFRPHRGAH | Y911856/ |
| | DAIAKVVDLYNQGYRRVVDLDLKAYFDNVNHDLMIKYLQQYIDDP | 603 . . . 2512/ |
| | WTLRLIRKFLTSGVLDHGLFAKSEKGTPQGGPLSPILANIYLNEL | Lactobacillus |
| | DKELTRRGHHFVRYADDCNIYVKSQRAGERVMRSITQFLEKRLKV | reuteri/ |
| | KVNPDKTKVGSPLRLKFLGFSL | BacterialC |
| 8517 | GIDGMPVEDLESHLRHHWPTLRQSLLDGTYQPKPVKRVEIPKGDG | >DS_Gfid\|42345 |
| | TKRALGIPTVIDRFVQQIIAQALSALWEPHFHPSSFGFRPARSAQ | 729\|locus\|VBIGa |
| | QAVKYVQTLQREKYEWVVDLDLKSFFDEVNHDRLIARLKTRVEDK | mPro61291_151 |
| | VLLRLINKFLHAGINANGILLRSEKGVPQGGPLSPILANIVLDEL | 7\|[gammaproteo |
| | DWELEHRGHKFARYADDCNIMVKSKAAGERVMKSIRRFLETTLRL | bacterium sp. |
| | RVNDQKSAVDRPTKRNFLGFTF | HdN1] |
| 8518 | GVDGMQVKELRYWFSNNHQKLIEQLKEGNYRPMTIKGQEIPKPGG | >DS_M.sp. I1/AF |
| | GVRQLGIPTVQDRLVQQAIAQQLSKRYDPTFSQYSYGFRKGRNAH | 339846/ |
| | QALRQAGAYVKEGFNYVVDLDLEKFFDKVNHDRLMWLLGRRISDK | 29388 . . . 31287/ |
| | RVLKLIGKFLRSGILIGGLENQRISGTPQGSPLSPLLSNIVLDEL | Microscilla |
| | DKELERRGHRFVRYADDMILLVRSQEAAERAYSSITSFIENRLLL | sp./BacterialC |
| | KVNKDKSRICRPYQLNFLGHSI | |
| 8519 | GIDGVTTAEWPEHARAHWPATREQIEAGRYRPQPVRRVDIPKPDG | >DS_N.e.I1/AL9 |
| | GQRQLGIPTVTDRVIQQAIAQVLIPIFDPGFSASSFGFRPGRNAH | 54747/ |
| | QAIRQVQAHVKAGYRWAVDLDLARFFDNVNHDLLMSLLSRSIADK | 2285095 . . . |
| | RLLALIGRYLRAGVLVGEHPQPSEVGTPQGGPLSPLLANVLLHQF | 2287101/ |
| | DLELERRGHRFARYADDVIILVKSRRAAERVMQSLTYFLQSTLKL | Nitrosomonas |
| | TVNLAKSQVAPMSECSFLGFTL | europaea/ |
| | | BacterialC |
| 8520 | GVDGVTIDAFPERFRPLWGDIRASLATGTYQPQPVLRVEIPKPTG | >DS_G.s.I1/AE0 |
| | GTRPLGIPTVLDRLIQQATAQVLTPIFDPEFSASSFGFRPGRSAH | 17180/ |
| | NAVRQLREYLRQGYRIAVDIDLAKFFDTVNHDLLMTMVGRRVRDK | 1028657 . . . |
| | RVLTLIGRYLRAGVEVDGRLEKTRMGVPQGGPLSPLLANILLDHL | 1030564/ |
| | DKELESRGHKFVRYADDFVILVKSERAGERVMGSVRKYLTNKLKL | Geobacter |
| | TVNEDKSKVARSGDLSFLGFVF | sulfurreducens/ |
| | | BacterialC |
| 8521 | GIDGMTIEAFPLWMQQGGWQRCKSLLERGEYNPSAVRRVEIDKPD | >DS_Sh.ba.I2/C |
| | GGKRKLGIPNVIDRVIQQAIAQILTPLFDPFFSANSFGFRPNRNA | P000563/ |
| | KQAVLQVRDIIKQKRKFAVDVDLSKFFDRVNHDLLMTQLRIKVQD | 2137684 . . . |
| | KRLLALIGKYLRAGVTVNDQFEASFEGVPQGGPLSPLLSNIMLDS | 2139633/ |
| | LDKELESRGHKFARYADDFIILVKSIRAGERVLKSITRYLATKLK | Shewanella |
| | LVVNEQKSQVVEVGQSKFLGFTF | baltica/ |
| | | Bacterial |
| | | C |
| 8522 | GIDGMNIDEFPAWVRSGNWKALKQQLVTGCYQPSPVRRVEIAKPD | >DS_P.ae.I1/AY |
| | GGTRQLGIPTVTDRVIQQAITQVLTPIFDPEFSEHSFGFRPGRNG | 029772/ |
| | QQAVKQVQSIIKEGRRFAVDVDLSKFFDRVNHDLLMTRLGDKVKD | 3515 . . . 5441/ |
| | KRLLRLIKRYLRAGFIDNQFKGESRVGVPQGGPLSPLLANIMLDS | Pseudomonas |
| | LDKELEKRGHKFARYADDFTILVKSQRAGERVLRSISQYLQSRLK | aeruginosa/ |
| | LVVNTDKSRVVKTNESQFLGFTF | BacterialC |
| 8523 | GVDNMPVTALKGYLQEEWPRIREELLTGTYHPQPVRKVEIPKPGG | >DS_Ge.ur.I2/C |
| | GTRMLGIPTVLDRLIQQAVHQVLSPLFDPGFSISSHGFRPGRSAH | P000698/ |
| | QAIKAARKYVESGLRWVVDIDLEKFFDRVHHDTLMSLVKRKVGDR | 242469 . . . 244398/ |
| | LVLSLIDSYLKAGILEGGVTSPRLEGTPQGGPLSPLLSNILLDEL | Geobacter |
| | DKKLERRGHKFCRYADDANIYVATRRSGERVMASITGYLSERLKL | uraniireducens/ |
| | TVNQGKSAVDRPWKRSFLSYSM | BacterialC |
| 8524 | GADGMTVADLAGYVKQYWPTLKARLLAGEYHPQAVRAVEIPKPQG | >DS_P.a.I1/U77 |
| | GTRQLGIPSVVDRLIQQALQQQLTPIFDPLFSDYSYGFRPGRSTH | 945/1 . . . 1919/ |
| | QAIEMARAHVTAGHRWCVELDLEKFFDRVNHDILMACIERRIKDK | Pseudomonas |
| | CVLRLIRRYLEAGIMSGGVVSPRQEGTPQGGPLSPLLSNILLDEL | alcaligenes/ |
| | DRELERRGHRFVRYADDANIYVRSPRAGERVLVSVERFLRERLKL | BacterialC |
| | TVNRKKSQVARAWKCDYLGYGM | |
| 8525 | GVDEKDIEATRLYLRENGQEIIQLIREGKYKPQPVRRVEIPKANG | >DS_B.sp. I1/NZ |
| | GKRQLGIPTVTDRVIQQAVVQRLTPIFERQFSHFSYGFRPNKSAH | _AAOX01000004/ |
| | QAIEQARQYIEEGYNFVVDMDLEKFFDRVQHDKLMSLIAKTISDK | 96386 . . . 98244/ |
| | PTLKLIRRFLQAGVMVNGVVITNREGTPQGGPLSPLLSNIILNEL | Bacillus |
| | DKELEKRGHKFVRYADDCNIYVKSIKAGERVKQGVTEFLERKLKL | sp./BacterialC |
| | KVNEEKSAVGKPSARTFLGVSF | |
| 8526 | CGVDGMKVDELLQYLKQNGKTLIASIFNGKYCPKAVRRVEIPKPD | >DS_C.a.I1/AE0 |
| | GGIRLLGIPTVVDRTIQQAISQVLTPIFEKTFSENSYGFRPKRSA | 01437/ |
| | KQAIKKAKEYMEEGYKWVVDIDLAKYFDTVNHDKLMALVARKIKD | 3710916 . . . |
| | KRVLKLIRLYLQSGVMINGVVSETERGCPQGGPLSPLLSNIMLTE | 3712835/ |
| | LDRELEKRGHKFCRYADDNNVYVRSKKAGDRVMRSITRFIENKLK | Clostridiuma |
| | LKVNKEKSAVDRPWRRKFLGFTF | cetobutylicum/ |
| | | Bacterial |
| 8527 | GVDEMDVKSLRLHLHENWTSIRNEIIEGSYFPKPVRRVEIPKPNG | >DS_B.h.I1/APO |
| | GVRKLGIPTVMDRFLQQAIAQILTQLYDPTFSERSFGFRPHRRGH | 01507/ |
| | NAVRQAKQWMKEGYRWVVDIDLEKFFDKVNHDRLMRKLSSRIQDP | 130149 . . . 132031/ |
| | RVLQLIRRYLQTGVMERGLVSPNTEGTPQGGPLSPLLSNIVLDEL | Bacillus |
| | DNELEKRGLKFVRYADDCNIYVRSKRAGLRIMESVTSFIENRLKL | halodurans/ |
| | KVNREKSAVDRPWNRKFLGFSF | BacterialC |
| 8528 | GIDEMSVKFLRRHLYDNWDSLRENLRKGTYTPSPVRRVEIPKPSG | >DS_O.i.I1/BA0 |
| | GVRMLGIPTVTDRFIQQAIAQVLHTIFDPSFSEHSYGFRPNRRGH | 00028/ |
| | DAVRKARGFIKEGYRWVIDMDLEKFFDKVNHDKLMGVLAKRIKDK | 2785523 . . . |
| | ELLRLIRKYLQSGVMINGIVVSSEEGTPQGGPLSPLLSNIILDDL | 2787411/ |
| | DKELEERGLRFVRYADDCNIYVRTKKAGNRVMNSITTFIEEKLRL | Oceano |
| | KVNKEKSAVDRPWKRKFLGFSF | bacillusiheyensis/ |
| | | BacterialC |
| 8529 | GVDGLGIVETAEHLKTAWPGIRAQLLAGTYRPDPVRRVLIPKPGG | >DS_P.s.I1/AE0 |
| | GERKLGIPTVTDRLIQQALLQVLQPLLDPDFSNHSYGFRPERSAH | 16853/ |
| | QAVLAAQQYIHSGRQIVVDVDLEQFFDCVEHDVLIARLGRKVKDR | 2381076 . . . |
| | DVLRLIRAYLNSGALIEGMVMTSTRGTPQGGPLSPLLANVVLDEV | 2382906/ |
| | DKELERRGHCFVRYADDANVYVRSPKAGQRVMALLRRLYGRLGLR | Pseudomonas |
| | VNESKSAVASAFGRKFLGFSF | syringae/ |
| | | BacterialC |
| 8530 | GVDGLDIGQTARHLVTAWPVIREQLLKGTYRPDPVRRVTIPKPDG | >DS_B.f.I1/NZ_ |
| | GERELGIPTVTDRLIQQALLQVLQPILDPTFSEHSYGFRPGRRAH | AAAC01000271/ |
| | DAVLAAQSYVQSGRRIVVDVDLEKFFDRVNHDILIDRLKRRIDDA | 24723 . . . 26575/ |
| | GVIRLVRTYLNSGIMDDGVVQQRDQGTPQGGPLSPLLANVLLDEV | Burkholderia |
| | DKELERRGHCFARYADDANVYVRSRRAGERVMALLRRLYGRLRLK | fungorum/ |
| | VNETKSAVASVFGRKFLGYSL | BacterialC |
| 8531 | GVDGRTVQQTGEDLKTQWPDIRRGLLDGTYRPSPVRRVGIPKLGG | >DS_Bfid\|19041 |
| | GTRELGIPTVVDRLIQQALLQVLQPLIDPTFSEHSYGFRPGRSAH | 3778\|locus\|VBIV |
| | QAVQAARQYVEQGRRVVVDVDLGKFFDRVNHDILMDRLGKRIADK | arPar264937_3261\| |
| | AVLRLIRHYLNAGIMAHGVMQMRVEGTPQGGPLSPPLLANVLLDE | [Variovorax |
| | VDRALERRGRKFVRYADDCNVYVKSERAGQRVLDGVRACYAKLRL | paradoxus |
| | KVNETKTAVATAWGRKFLGYCL | B4] |
| 8532 | GVDGLTIEETPEYLKTHWSRIRLELLNGTYRPQAVRRVEIPKPTG | >DS_Bfid\|22964 |
| | GMRELGIPTVLDRLIQQALLQVLQPMIDLTFSEFSYGFRPGRSAH | 000\|locus\|VBIPo |
| | DAVLQAQRYVQEGFQVVVDVDLEKFFDRVNHDILMDRLAKRIADK | 1Sp102244_5444 |
| | AVLRLIRQYLQAGIMAGGVVMDRSEGTPQGGPLSPLLANVLLDEV | \|[Polaromonas |
| | DLDLQRRGHRFARYADDCNVYVRSQKAGERVLLSLRKLYEKLHLK | sp. JS666] |
| | VNEKKTEVGPVFGRKFLGYCL | |
| 8533 | GVDKLTVQELKPWLKQHWLSVKGTLIAGSYLPRAIRKVDIPKPNG | >DS_fid\|190304 |
| | DVRTLGIPTVVDRLIQQAIAQTLSPYVEPSFSNSSYGFRPNRNAW | 141\|locus\|VBISe |
| | QAVRQAQQYIQSGKRWVVDMDLEKFFDRVDHDILMSRLARTIKDK | rSp8482_4636\| |
| | RLLKLIRRYLEADMVEGKEVIKRDKGMPQGGPLSPLLSNILLDEL | [Serratia |
| | DKELERRGHSFCRYADDCNIYVSSQKAGKHAQKDISEFLMNTLKL | sp. |
| | QVNVRKSAVARPWERKFLGYSF | ATCC39006] |
| 8534 | GVDNLSVGELKGWLKQHWASVREALLQGNYVPQAIRQVEIPKPDG | >DS_fid\|190303 |
| | GVRILGIPTVVDRLIQQAIQQHLTPDYEPEFSDSSYGFRPGRNAG | 244\|locus\|VBISe |
| | QAVQQAQSYMQSGRRWVVDLDLEKFFDRVNHDILMARLSWKIKDT | rSp8482_4195\| |
| | RLLKLIRRYLEADRVAGSEITRRREGMPQGSPLSPLLSNILLTDL | [Serratia sp. |
| | DRELERRGHKFCRYADDGNIYVCSRQAGEHAMKEISHYLENKLRL | ATCC39006] |
| | KVNAHKSAVDRPWKRKFLGYSV | |
| 8535 | GVDALEVTALRDWLKVSWPSVRAALLGGQYIPQSVRAVDIPKPSG | >DS_Bfid\|21533 |
| | GVRTLGIPTVVDRLIQQALLQVLQPLYEPGFSESSYGFRPRRSAQ | 277\|locus\|VBICu |
| | QAVLQAQRYVQEGRRWVVDIDLEKFFDRVNHDILMSRVARQVKDV | pTai42494_3259 |
| | RVLKLIRRYLEAGLMRGGVVEARRQGTPQGGPLSPLLSNILLTDW | [Cupriavidus |
| | DRELEKRGLAFCRYADDCNIYVRSQAAGQRLLAGMMTFLAERLNL | taiwanensis] |
| | QVNEAKSACARPWARKFLGYSL | |
| 8536 | GVEGMSVSELPDYLKHHWPELKAQLLSGSYCPSPVRRVTIPKPGG | >DS_Gfid\|21803 |
| | GERLLGIPTVVDRFVQQATMQVLQRQWDASFSDSSYGFRPGRSAH | 083\|locus\|VBIDi |
| | QAVKQAQGYIGSGHHWVVDLDLEKFFDRVNHDVLMSRVAKRVSDK | cZeal11179_3566\| |
| | RVLSLIRGFLNAGVMEAGLVSPVTEGMPQGGPLSPLLSNLLLDDF | [Dickeyazeae |
| | DKELEKRGLKFARYADDCNIYVKSERAGNRVMEGLTHWLSRKLKL | Ech1591] |
| | KVNAKKSAVAHPAMRKFLGYSF | |
| 8537 | CGVDGMTVQALPAFLREQWPSIRATLLNGTYKPQPVRRVEIPKPD | >DS_Bu.vi.I2/C |
| | GGGVRKLGIPCALDRFVQQAVLQVLQRQWDPTFSEASYGFRPGRS | P000617/381828 |
| | AHQAVAKAQSYIQSGYRWVVDLDLEKFFDRVNHDILMSRVARRVS | . . . 383697/ |
| | DRRVLKLIRSFLTAGVMEHGLVGATDEGTPQGGPLSPLLSNLMLD | Burkholderia |
| | DLDRELGRRGLRFVRYADDCNVYVRSERAGQRVMVGLKAFLTGKL | vietnamiensis/ |
| | KLKVNEAKSAVARPHTRKFLGFSF | Bacterial |
| 8538 | GVDGMTVIGIKDYLKQHWPAIRGQLLSGTYEPKPVRRVEIAKPDG | >DS_So.us.I3/C |
| | GVRKLGIPTVLDRFIQQAVMQVLQRRWDRTFSDYSYGFRPGRSAQ | P000473/ |
| | QAVAQAQQYIAEGHGWCVDLDLEKFFDRVNHDKLMGQIAKRIADK | 9594438 . . . 9596378/ |
| | RLLKLIRAFLNAGVMENGLVSPSVEGTPQGGPLSPLLSNLVLDEF | Solibacter |
| | DRELERRGHRFVRYADDCNIYVRSERAGQRVMESITQFITQKLKL | usitatus/ |
| | KVNETKSAVARPQERKFLGFSF | BacterialC |
| 8539 | GVDGMTVDDLPTYLKANWLTIRAQLLDGTYKPQAVRRVEIPKASG | >DS_Br.sp. I1/C |
| | GVRLLGIPTVVDRFIQQAVLQVLQGEWDRTFSDASYGFRPGRSAH | P000494/ |
| | QAVTKAQAYIASRHRIVVDIDLEKFFDRVNHDILMGLVAKRVADK | 6816299 . . . 6818172/ |
| | RLLKLIRGFLTAGVLEGGLVSPTEEGAPQGGPLSPLLSNLMLDVL | Bradyrhizobium |
| | DKELERRGHRFVRYADDCNIYVRSRKAGERVMASIETFLERCLKL | sp./BacterialC |
| | KVNRAKSAVARPNHRKFLGFSF | |
| 8540 | GQDGITFEHIEERGRAGFLGAVAEELRTGTYRPRPYRRREIPKEG | >DS_Mx.xa.I1/C |
| | GKVRVISIPSIRDRVVQGALRLVLEPIFEADFSGSSFGARPGRSA | P000113/ |
| | HEAIDTVRQGLRRRRHRVVDVDLKAYFDSIRHAPLLERVARRVQD | 2433780 . . . 2435766/ |
| | GEVLALVKQFLRSTGDRGIPQGSPLSPLLANIALNDLDHVLDRGR | Myxococcus |
| | GFLTYARYLDDMVVLAPDSEKGRRWAARALERIRQEAEALGVSLN | xanthus/ |
| | KEKTRTVTMTDRNASFAFLGFDF | BacterialF |
| 8541 | GIDDLSFEDIEASGRIVFLAEIQADLKTGRYEPKPNRRVEIPKSN | >DS_Bfid\|58553 |
| | GKVRVLQVPCIRDRVVQGALKLILEAVFEADFCPNSYGFRPKRSP | 039\|locus\|VBICu |
| | HRALAEVRRSVLRRMSTVVDVDLSRYFDTIQHSTLLGKIAKRIQD | pNec201015_1883\| |
| | PQVMHLVKQVIKAAGKVGVPQGGPFSPLAANIYLTEIDWMLDEIR | [Cupriavidus |
| | RKTAQGPYEAVNYHRFADDIVITVSGHHTKRGWAERALLRLREQL | necator |
| | VPLGVELNTEKTTVVDTLHGEAFGFLGFDL | N1] |
| 8542 | GIDGKSFADIELEGVIPFLTGIQEELQAGIYQPQANRKVEIPKTN | >DS_B.th.I3/DQ |
| | GKMRTLQIPCIRDRVVQGALKLILEAIFEADFCPNSYGFRPKRSP | 363750/ |
| | HQALAEVRRSILRRMTIIIDVDLSRYFDTIRHNILLEKIAKRVQD | 30070 . . . 32039/ |
| | PQVMHLVKQVIKATGKIGVPQGGPFSPLAANIYLNEVDWTFDTIR | Bacillus |
| | RKTADGNYEAVNYHRFADDIVIAVSGHSSKSGWAELALRRLWEQL | thuringiensis/ |
| | KPLGVELNLEKTQMVNVLKGESFGFLGFDL | Unclassified |
| 8543 | GIDGVTFESIETEGSRKYLQRIRHELITKTYSPNRNRRKEIPKSG | >DS_W.e.I5/AM |
| | EKFRTLNIPCIRDRIVQTALKLILEPIFESDFQKGSYGYRPKRNA | 999887.1/ |
| | HEAVQKVTEAAIKGNTKVIDVDLKSYFDSVRHHILMEKIAKRIND | 284826 . . . 286812/ |
| | KEIMRMIKLILKIGGKRGMAQGSPLSPLLSNIYLNEVDKMLEKAK | Wolbachia |
| | EVTKEGKYQRMEYARWADDLVILIREYPKREWLERAVYRRLEEEL | endosymbiont |
| | AKLEVRVNEEKTKVINLKKGETFSFLGFDF | /Unclassified |
| 8544 | GVDGITFEDIEGIGVLKYLKKIREELVNETYKPQENRKQEIPKGN | >DS_gi\|3023920 |
| | GKVRVLGIPTIKDRIVQGALKLILEPIFEADFQESSYGYRPKRTA | 22\|ref\|WP_01327 |
| | HQAVKKIEKAIVSGKRKVIDLDLSSYFDTVKHHILLAKIAKRVID | 8223.1\|Acetohalobium |
| | KEVMHLIKLMLKASGKEGVPQGGVISPLFANLYLNEVDRMLERAK | arabaticum |
| | EVTKSKGKYTELEYARFADDIVIAVSSHPSMNWLLSKVIQRLKEE | DSM5501 |
| | LDKIKVKVNKEKTKVVNLEKGERISFLGFTL | |
| 8545 | GIDGVTFEAIEESGVEQFLGEVRKELVSGSYRPLKNRRKAIPKGD | >DS_Ma.sp. I2/C |
| | GKERVLGIPSIRDRVVQGALKLILEPIFEADFQSGSYGYRPKRMA | P000471/ |
| | HQAVNRVAIAIAQGKTQVIDADLKSYFDTVQHDLALRKVSERVDD | 2464047 . . . 2465973/ |
| | DQVMHLLKLIFKTSGKRGVPQGGVISPLISNLYLNEVDKMLERAK | Magnetococcus |
| | EVTRKGKYTHIEYARFADDLVILVDGHHRWNGLARKVYQRLGEEL | sp./Unclassified |
| | AKLKVQLNLEKTRVVDLTRGEDFTFLGFNI | |
| 8546 | GIDGITFDNIEASGIEIFLQQIQKELISGTYWPTQNRRKEIPKGD | >DS_gi\|2585150 |
| | GKYRILGIPTIRDRVVQGALKLILEPIFEADFQEGSYGYRPKRNP | 71\|ref\| |
| | HQAIDRVAKAVVENKTRVIDLDLRSYFDTVRHDLLLKKVAKRVND | YP_00319 |
| | ENVMRLLKLILKASGKRGVPQGGVISPLLANLYLNEVDKMLEKAK | 1293.1\| |
| | EVTRHEQYTHIEYARFADDIVILIDAYPKWNWLEKAVYQRLLEEL | Desulfotomaculum |
| | TKLDVQLNEEKTRIVNLANGESFGFLGFDF | acetoxidans |
| | | DSM771 |
| 8547 | GIDRITLEEVEEYGVARLLDELAVELKEGSYRPLPARRVFIPKPG | >DS_Rh.sp. I1/C |
| | TVEQRPLSIPSVRDRIVQAAWKLVAEPVFEADFLPCSFGFRPRRG | P000432/ |
| | AHDALQVLIDESWRGCRWVVETDIANCFEAIPIEKLMQAVEERVC | 23005 . . . |
| | DQPFLKLLRVMLRAGVMEEGQVRRPVTGTPQGGVASALLCNVYLH | 25058/Rhodococcus |
| | RLDRAWDVDEHGVLVRYADDALVMCRSRRQAEAALTRLRELLADL | sp./Unclassified |
| | GLEPKEAKTRIVHLRVGGEGVDFLGFHH | |
| 8548 | GVDHVSMEAIASNPRKYLYPLWNRLSSGSYFPPPVKLVPIPKGDG | >DS_UMB_I1/A |
| | KERMLGIPTIIDRVAQEVIKAELEVIVEPRFHPSSFGYRPHKSAH | Y075117/120 2 |
| | EALEQCAKNSWERWYVVDLDIKGFFDNIDHEKMMGILRKHTNKKH | 136/uncultured |
| | ILLYCDRWLKTPMQDRVGGVQARMKGTPQGGVISPLLANLYLHEA | marine_bacterium |
| | FDQWISTTQPRIVFERYADDIVIHTRSMEQSHFILDKLKARLKSY | |
| | SLELHPDKTKIVYCYRTARFHKEGKEIPVSFDFLGFTF | |
| 8549 | GVDGMTIEAFEHNLARNLYKIWNRLSSGCYMPPPVKRVEIPKSDG | >DS_D149_(ZP |
| | KTRPLGIPTVSDRVAQMAVKMILEPQWDPLFSDSSFGYRPGKSAH | 06641622.1_) |
| | DAVAQAKANCWKYEWVIDLDIRGFFDNLDHALLLKAVDHLHPAPW | Serratia |
| | VRLCIVRWLKAEIIFPDGHRHSPEKGTPQGGVISPLLANLFLHYT | odorifera |
| | QDKWLEKHYPNNSWERYADDSIIHCRSRREAGLLLSQLRERMKAC | DSM4582416 bp |
| | GLELHPEKTRIVNCHPLTRRKNDGHYSFDFLGFTF | |
| 8550 | GADNVCIDMFEHNLENELYKLWNRMSSGSYMAPPVKRVEMAKADG | >DS_Ce_ja_I1/C |
| | KLRPLGIPTVADRVAQMVVKMTLEPEWDSKFHASSFGYRPRRSAH | P000934_1/3788 |
| | HAVQAAKINCWKYSWVIDLDIKGFFDNLNHDQLQKFVAQATDDPW | 874_3790736/ |
| | CKLYIKRWITAGVQMPGGELHKTAKGTPQGGVISPLLANLYLHKV | Cellvibrio |
| | FDSWMQKYFPQNPFERYADDIVCHCRTEHEAEQLLSAISRRMQRF | sp. |
| | DLTLHPEKTKIVYCGRRKIERTKAQSFDFLGFTF | |
| 8551 | GPDGVTVEQFEANVKDRLYVLWNRMSSGSYFPGPVGAVEIPKKGV | >DS_Fr_sp_15/C |
| | KGGARTLGIPNVVDRVAQTVLKLALEPKVEPVFHRDSYGYRPGRS | P000820_1/4042 |
| | QRQALEVCRKRCWSHDWVVDLDVRKFFDTVPWEKLLKAVAYHTDQ | 207_4044207/ |
| | KWVLMYVERCLKAPTKHADGTLQERTMGTVQGGPFSPLAANIYLH | Frankia |
| | WGLDAWMAREFPTVPFERWADDVVFHCVSLEQAREVRDAVVARLV | sp. |
| | EVGLEAHPDKTRIVYCKDSNRGGDYENTSFTFLSYTF | |
| 8552 | GIDTVSIEQFDESLSKNLYKLWNRMASGSYFPPAVKEVEIPKKDG | >DS_Zu_pr_12/ |
| | KVRKLGIPTISDRIGQMVVKMYLEPRLENVENPNSYGYRPNKSAH | CP001650_1/358 |
| | QALEQVRKNCWKMDWVIDLDIKGFFDNIDHHKMMLAIEKHVPERW | 9332_3591217/ |
| | VRLYIARWLASPVMTKSGNLVSNQGRGTPQGGVISPLLANLFLHY | Zunongwangia |
| | GLDKWLEQNDNTVKFTRYADDVIVNCKSQKHAEQTLEAIKSRMHQ | sp. |
| | IGLELHPEKTKIVYCRDYRRQEKYSNVKFDFLGYSY | |
| 8553 | GIDTQSLEQFEERLADNLYKIWNRMTSGSYHPKAVREVQIPKKSG | >DS_D182_(YP |
| | GYRGLGIPTVSDRVAQQVVKSYLEPKVEPSFHQDSYGYRPNKSAH | 003997451.1_) |
| | DALAKTVRNCGYYSWVVDLDIRGFFDNIDHELLMKAVRVYTDEKW | Leadbetterella |
| | IIMYIERWLEVGVVREGKVHKREKGTPQGGVISPLLANIFLHFVF | byssophila |
| | DKWMEKHHGNMPFERYCDDAIIHCTTWNQAVFIKNAVTKRMKECK | DSM17132410 bp |
| | LELNSEKTKIVYCKNSIHRESNPVPVSFTFLGHTF | |
| 8554 | GIDKVTLEDYEKNLRGNLYKLWNRMSSGSYFPPSVKLVEIPKSTG | >DS_B_t_14/AE |
| | GKRPLGIPTVSDRVAQMAVVMLITPSIEPCFHEDSYAYRPHRSAH | 015928/3254752 |
| | DAVGKARERCWKYAWVLDMDISKFFDTIDHELLLKALKRHTQEKW | /Bacteroides |
| | VLMYIERWLKVPYEKSDGSQVDRALGVPQGSVIGPVLANLFLHYT | thetaiotaomicron |
| | FDKWMEKNFPRVPFERYADDTICHCHSLKQAEYMQAMIQQRFECC | /BacterialD |
| | RLRLNEEKTKIVYCKSSRQKECYPNVTFDFLGFTF | |
| 8555 | GVDGVGLAGFESDLKGNLYRIWNRMSSGSYFPPPVKAVEISKEHG | >DS_D218_(ZP |
| | AGTRMLGVPTIGDRIAQTVVAARLEGVVEPKFHPDSYGYRPRKGS | _06415879.1) |
| | LDAVRKCRERCWKYDWVIDLDVRKFFDTVPWDRIIAAVEANTALP | Frankia |
| | WVLLYVKRWLAAPVRMPDGTLAERDRGTPQGSAVSPVLANLFMHY | sp. EUN1f417 bp |
| | AFDLWMVREFPACPFERYADDAVVHCKSLAQARFVLDRLRKRMEQ | |
| | VGVSLHPEKTRIVYCKDGKRRGSHEHTEFTFLGFTF | |
| 8556 | GVDGQSIDAFEKDLKNNLYRIWNRMSSGSYFPPPVRAVEIPKAHG | >DS_D154_(ZP |
| | GGVRVLGVPTVADRVAQTVVAMTLEPRMEQVFHDGSYGYRVGRSA | 06477373.1_) |
| | LDAVGACRQRCWQRDWVVDLDIQDFFGSCPHDLIVRAVEVNTDQP | Frankia |
| | WVVLYVRRWLTAPVCYPDGSLVTPDRGTPQGSAVSPVLANVFLHY | sp. |
| | ALDLWLAREFPGLPFERYVDDAVVHCATRRQAEQVRTAIGRRLEE | (symbiont of |
| | VGLRCHPAKTKVVYCKDSGRRGSHEHTSFTFLGYTF | Datiscaglomerata) |
| | | 421 bp |
| 8557 | GLDGLTMEAFEEDLKNQLYRLWNRMSSGSYFPPPVMRVEIPKSDG | >DS_Ma_sp_I3/ |
| | GVRGLGIPTIGDRIAQAVVKRYLEPLVEPKFHEDSYGYRPNRSAL | CP000471/78572 |
| | DAVRQARQRCWRDDWVLDLDISKFFDKLDHALVMRAVKRFTDCKW | 7/Magnetococcus |
| | VLLYIERWLKADVQLQDETILHREMGTPQGGVISPLLANIFLHLG | sp./BacterialD |
| | FDQWMKENYPHIHFERYADDIVVHCRSLKQLQWIKKRIEQRLKLC | |
| | KLSLNDKKTRVVYCKDSRRSGEWTCQSFDFLGYTF | |
| 8558 | GADEQTIKEFEEHLNNNLYKLWNRMASGSYFPKPVRAVAIPKKNG | >DS_D153_(ZP |
| | GIRILGIPTVEDRIAQMVAKMYFEPLVEPMFYNDSYGYRPNKSAI | _04856034.1_) |
| | QAVGQARERCFKRDWALELDIKGLFDNIKHGYLMYMVEKHTQIKW | Ruminococcus |
| | LILYIKRWLTVPFIMSDGSVAERRSGTPQGGVISPVLANLFLHYV | sp. |
| | FDDFMTKAYPNIWWERYADDGVLHCQSYKQAAFIKQKLEERFQQF | 5_1_39B_FA |
| | GLELNKEKTRIVYCKDNRRPQNYSCTQFTFLGYTF | A418 bp |
| 8559 | GVDGQSLEDFAGDLENHRYRLWNRLVSGSYFPPPVRRVEIPKAGG | >DS_B_j_I1/BA |
| | GIRPLGIPTVADRIAQMVVKRCLEPVLDGEFDPDSYGYRPGKSAH | 000040/2212569 |
| | QAIEQARKRCWQHDWVVDLDNKSFFDTIDHELLMRAVYRHTKADW | Bradyrhizobium |
| | IRLYIERWLKAPVEMPDGSVRARTTGRSQGGVVSPILANLFLHYV | japonicum/ |
| | FDVWMKGSYPHIPFERYADDIICHCRTRQEAEELKSALERRFADC | BacterialD |
| | HLLLHPEKTKVVYCADSNRRRSYPQIHFDFLGFSF | |
| 8560 | GVDGQTLESFGERLGPNLYKLWNRMSSGSYMPSSVRRVMIPKADG | >DS_Pa_de_I1/ |
| | GQRPLGIPTVTDRIAQEVVRLYLEPLVEPVFHRDSYGYRPERSAI | CP000491/19065 |
| | DAIRKARQRCWRYDWVLDMDIKGFFDTIDHELLLKAVRHHTDCRW | Paracoccus |
| | VLLYIERWLKAPVRMEDGSLVPQERGTPQGGVISPLLANLFLHYA | denitrificans/ |
| | FDRWLDRENPQVPFERYADDIICHCRTEDEARRLWQQVENRLAGC | Bacterial D |
| | GLTLHPQKTKIVYCKDTNRKGSFPTVAFDFLGYRF | |
| 8561 | GVDGQSIEEFEQDLSGNLYKLWNRLASGSYMPPAVRCVEIPKATG | >DS_D172_(YP |
| | GTRPLGIPTVADRIGQMVVKDALEPILEPCFHHDSYGYRPNKSAH | _003750926.1_) |
| | DALAVARQRCWRAAWVLDVDIKGFFDNIDHALLMKAVRKHIDCRW | Ralstonia |
| | ITLYIERWLTAPVQLPDGTSQARNKGTPQGGVISPLLANLFLHYV | solanacearum |
| | FDMWMVRNFPANGFERYADDVVIHSTSLKQVTMLRAQLTERLADC | 412 bp |
| | KLEMSPGKTKIVYCKDKRRKGGYPEISFDFLGYTF | |
| 8562 | GIDKISIEKYEKNLKNNLYKLWNRMASGTYFPKAVKAVEIPKKNG | >DS_D143_(AD |
| | GIRVLGVPTVEDRIAQMIVKLSMEKIIDPIFLNDSYGYRPNRSAH | O77309.1_) |
| | DAIKVTRSRCWKYDWVLEFDIKGLFDNINHKLLLKAVYKYAKYKW | Halanaerobium |
| | EILYIKRWLANPVSNNNKITKNTENGTPQGGVISPLLANLFLHFA | praevalens |
| | FDKWMEKRFPNNKWCRYADDGIIHCNSRAEAIFILNCLKERMKEC | DSM2228415 bp |
| | KLEIHPGKTKIIYCKDSNRKENNKLHEFTFLGYSF | |
| 8563 | GIDEVTLQEYENNLEDNLYKLWNSMSSGSYFPQAVRGVEIPKKNG | >DS_Al_me_I4/ |
| | GVRVLGVPSIDDRIAQNVMVSELNPKVEPIFYEDSYGYRENKSAI | CP000724/658338/ |
| | DAIEVTRKRCWEYDWLIEFDIVGLFDNINHDLLMKAVKQHTNEKW | Alkaliphilus |
| | VILYIERTLKVPMVMSDGIHVERTKGTPQGGVISAVLANLFMHYA | metalliredigens/ |
| | FDHWMTRKHSNNPWVRYADDGLIHSHSLKEAEVLLLKLGERFKDC | BacterialD |
| | HLEIHPNKTKIIYCKDDNRKQNHIHTNFDFLGYTF | |
| 8564 | GVDQESIEAFEKNLKGNLYKLWNRLSSGSYFPPPVKGVGIPKKTG | >DS_D115_(YP |
| | GIRMLGVPTVADRVAQTVGKETLEPLLEPIFHQDSYGYRPGRSAL | _911931.1_) |
| | DAVGVVRERCWKYDWVVEFDISKFFDTMNHELLMRAVRKHCQIEW | Chlorobium phaeo |
| | VLLYVERWLKAPMMSPEGDLVERTKGTPQGGVISPLLANLFLHYA | bacteroides |
| | FDRWVSENLPGVPFCRYADDGVLHCKSKEQAVLVMKKITKRFEAC | DSM266418 bp |
| | GLRVNPDKTRIVYCKDDKRKEDHPVTSFTFLGYTF | |
| 8565 | GVDRESLQAFETKLKDNLYKVWNRLSSGSYFPPPVRGVGIPKKSG | >DS_Pe_ph_I1/ |
| | GVRMLGVPTVADRVAQSVVKMVLEPILEPVFHEDSYGYRPGRSAH | CP001110/398581 |
| | DAIAVVRKRNWEYDWVVEFDIKGLFDNIDHELLMRALRKHCQTPW | Pelodictyon |
| | VFLYVERWLKAPMETPEGELIERTKGTPQGGVVSPLLANLFLHYA | phaeoclathratiforme |
| | FDRWVSENLPGVPFCRYSDDGVLHCKSKIQAELVKRKIGERFREC | /BacterialD |
| | GLELHPDKTQIVYCRDSNRKDEHPVNQFTFLGFTF | |
| 8566 | GIDDETIADFERNLPKNLYKLWNRMSSGSYFPPPVKAVEIPKASG | >DS_fid\|867055 |
| | GIRRLGVPTVSDRIAQTVVKLLIEPKLDALFHPDSYGYRPGRSAK | 94\|locus\|VBIPse |
| | QAIAITRERCWRYDWVVEFDIKAAFDHIDHELLMKAVRTHIKEDW | Put3905_0289\| |
| | ILLYIERWLVAPFEAADGVRIQRERGTPQGGVISPMLMNLFMHYA | [Pseudomonas |
| | FDAWMQRNSPNCPFARYADDAVVHCRSQRQAEHVMRSIASRLAVC | putida ND6] |
| | GLTMHPEKSKIVYCKDSNRRAGYPHVSFTFLGFTF | |
| 8567 | GVDGVTIEDFEKDLKNNLYKIWNRMSSGSYFPTPVAAVSIPKKSG | >DS_Vi_vu_I1/ |
| | GERVLGIPTVSDRVAQTVVRDKLEIMLEHHFLDDSYGYRVGKSAH | GQ292873_1/36 |
| | DAIEVTRRRCWQYDWVLEFDIKGLFDNIRHDLLMKAVKKHVQLAE | 20 __ 5549/ |
| | ESQSRDYQWITLYIERWLVAPLQKADGTQTERELGTPQGGVVSPV | Vibrio |
| | LANLFLHYVFDKWLEKNYPDNPWCRYADDGLVHARTKPKAEKLRD | Vibrio |
| | ELAKRFKECGLEMHPIKTKIVYCKDDIRRGSGKHIEHKQFDFLGY | vvulnificus/ |
| | TF | BacterialD |
| 8568 | GVDGVTIEQFEKDLKGNLYKIWNRMSSGAYFPPPVRAVSIPKKSG | >DS_D216_(ZP |
| | GQRILGVPTVADRVAQTVVKEIIEPALDAIFLADSYGYRPDKSAL | _08074908_) |
| | DAVGVTRERCWKFDWVLEFDIKGLFDNIDHTLLMRAVRKHVACPW | Methylocystis sp. |
| | ALLYIERWLTAPMMQEDGTLIERTRGTPQGGVVSPVLANLFMHYT | ATCC49242417 |
| | FDLWMARTFPHLRWCRYADDGLVHCRSEREARIVWEALASRMAEC | bp |
| | RLELHPTKTKIVYCKDDRRKANFENVAFDFLGYCF | |
| 8569 | GVDGQTLEIFEKDLAANLYKIWNRMSSGTYFPPPVRAVSIPKKAG | >DS_RmInt1_(Y |
| | GERVLGVPTVSDRIAQMVVKQMIEPDLDSLFLPDSYGYRPGKSAL | 11597.2_) |
| | DAVGVTRQRCWKYDWVLEFDIKGLFDNLPHDLLLKAVRKDVKCNW | Sinorhizobium |
| | ALLYIERWLTAPMEKNGEVIERSRGTPQGGVVSPILANLFLHYAF | meliloti |
| | DLWMTRTHPDLPWCRYADDGLVHCQSEQQAEALRVELSSRLAACG | 419 bp |
| | LQMHPTKTKIVYCKDQRRREAYPNVTFDFLGYQF | |
| 8570 | GVDGQTIEQFEADLKGNLYKIWNRMSSGSYFPPPVRAVPIPKKTG | >DS_RmInt2_(Y |
| | GQRILGVPTVSDRIAQMVVKQLIEPELDQIFLKDSYGYRPNKSAL | P_007194308) |
| | DAVGITRQRCWKYDWVLEFDIKGLFDNISHELLLKAVRKHVKCKW | Sinorhizobium |
| | ALLYIERWLTAPMEQDEQRIERDCGTPQGGVISPILSNLFLHYAF | meliloti |
| | DLWMDRTHPDLPWCRYADDGLVHCRSEQEAEAVKAALQARLAECQ | 419 bp |
| | LEMHPTKTKIVYCRDSKRRGQHPNVTFDFLGYCF | |
| 8571 | GIDKQSLADFDKRLVDNLYKIWNRLSSGSYFPPAVKAVAIPKKLG | >DS_E_c_12/X7 |
| | GERILGIPTVSDRIAQTVVKLAFEPQVEPHFLADSYGYRPNKSAL | 7508/518 |
| | DAIGVTRKRCWYYDWVLEFDIKGLFDNIPHELIMKAVDKHNPARW | Escherichia |
| | VKLYIQRWLTAPMVMSDGEVRARTMGTPQGGVISPLLANLFMHYV | coli/ |
| | FDKWLAKYYPKVPWYRYADDGILHCHSEAEATEMREVLRKRFSEC | BacterialD |
| | GLEMHPEKTRVIYCKDGSRKGDYEHTMFDFLGYTF | |
| 8572 | GVDDENIAAFESDLTNNLYKIWNRMSSGCYFPPSVKAIEIPKKSG | >DS_M_a_I53/A |
| | GTRILGIPTVLDRVAQMVTKIYLEPQLEPLFHPDSYGYRPGKSAA | E011185/2451M |
| | DALAATRKRCWRYNWLLEFDIKGLFDNINHDLLMKQVSMHTDKPW | ethanosarcina |
| | IILYIQRWLKAPFQMADGTVNERTKGTPQGGVVSPLLANLFLHYA | acetivorans/ |
| | FDQWMDSHHRYNPFERYADDSVIHCRSREEAERLWIELDKRLSEF | BacterialD |
| | GLELHPSKTRIVYCKDDDRQGDYPETKFDFLGYTF | |
| 8573 | GIDEQSIDEFERNLKDNLYKVWNRMSSGSYIPPAVKAVEIPKKAG | >DS_D152_(YP |
| | GIRTLGIPTVADRIAQMTVKLYFEPLVEPFFHEDSYGYRPKKSAI | 003428936.1_) |
| | QAIETTRKRCWKYNWVLEFDIKGLFDNIDHELLMRAVDKHTDIEW | Bacillus |
| | VKLYIKRWLTAPFQTKEGIKERTSGTPQGGVISPVLANLFLHYAF | pseudofirmus |
| | DKWMAINHPRNPFARYADDAVIHCKTEEEAKRVLESLNQRMNECK | OF4414 bp |
| | LELHPSKTKIVYCKDADRREDHKNITFDFLGYTF | |
| 8574 | GIDEVSLEEFEADLDNNLYKIWNRMTSGSYFPPPVKAIEIEKKSG | >DS_WP_01473 |
| | GKRVLGIPTVGDRVAQMVAKIYLNPLVDPYFHKDSYGYREGKSAI | 1166 Mesotoga |
| | DALEVTRQRCWRYDWVLEFDIKGLFDNIDHELLMRAVKKHVKIPW | prima |
| | LILYIERWLKAPFIQANGRVEERSKGTPQGGVISPVLANLFMHYA | 411 bp |
| | FDKWMERTHPDKPFARYADDGVIHCRTLEEARLLLESLKERMEEC | |
| | KLKLHPEKTRIVYCKDDKRKGEYPNTSFDFLGYTF | |
| 8575 | CGIDGETVFNFHLNLELNIEFLHDKLKTNGYEPSPVRRVEIQKPD | >DS_Al.or.I2/CP |
| | GGVRLLGIPTVKDRVVQQAIVNIIEPIFDKTFHPSSYGYRPNHSQ | 000853/2108190 |
| | HGAVAKAERFMNKYGLEHVVDMDLSKCFDTLDHEIMMKAVSERIS | . . . 2110275/ |
| | DGRVLKLIEKFLKAGVMHSDNFSRTEVGSPQGGVISPLLSNIYLN | Alkaliphilus |
| | QFDQRMMSKGIRIVRFADDILIFAKDKKTAGNYKAYATQVLENEL | oremlandii/ |
| | KLKVNNEKTKLTNVNEGVEFLGFVI | Bacterial |
| 8576 | GIDGQSVKDFAESLDVNLDRLLTELREKSYQPQPVRRVEIPKENG | >DS_D.p.I1/CR5 |
| | GIRLLGIPAVRDRVVQQALLDILQPIFDPDFHPSSYGYRPGRSCH | 22871/ |
| | QAITKATMFIRKYDRKWVVDMDLSKCFDTLDHDLILSSLSRRIKD | 6124 . . . 8213 |
| | GSILGLLKKILKSGVMTDEGWQASEVGSPQGGVISPLIANIYLDQ | /Desulfotalea |
| | FDQFMKKRGHRIVRYADDILILCSSKSAAKNALLQASCFLEKGLL | psychrophila/ |
| | LTVNREKTHICHSWSGVAFLGVSI | BacterialC |
| 8577 | GVDGVTVRQIRQRGEVGVFLAGIAASLRDGTYRPAPVRRVLIPKP | >DS_gi\|3171240 |
| | GGKSRPLGIPTVTDRVVQQSLRMVLEPIFEADFLPVSYGFRPKRR | 20\|ref\|YP_00409 |
| | AHDAVAEIHFYAGRGYRWVLDADIEGCFDHIDHTALLGLVRERIK | 8132.1\| |
| | DKKTVALVRAFLKAGVLSDLGLEAAAGEGTPQGGIISPLLANIAL | Intrasporangium |
| | SVLDEAIMAPWAQGGDQSTQTGRAKRRYHGLGNWRIVRYADDFVI | calvum |
| | MTNGSRDDVLALKEQAAEVLARVGLRLSESKTRVTHLSEGIDFLG | DSM43043 |
| | FHI | |
| 8578 | GVDGRTAASIVARIGIPEYLDGLRSALKDRSFRPLPVRERMIPKA | >DS_gi\|3361776 |
| | GGKLRRLGIATITDRVVQASLKLALEPIFEADFLPCSYGFRPMRR | 63\|ref\|YP_00458 |
| | AHDAVAEIRYLTSKPRCYEWIVEGDIKACFDEISHTSLTGRVRAR | 3038.1\|WP_0138 |
| | IGDRRVLALVKAFLKSGILVEDRLVRPTTAGTPQGSILSPLLSNV | 73076.1\|Frankia |
| | ALSVLDEHVARSPGGPGTGKTEKAKRLRHGLPNFKLVRYADDWCL | sp. Symbiont of |
| | VIKGTKADAEALREEIAGVLSTMGLRLSREKTLITHIDDGLDFLG | Datisca |
| | WRI | |
| 8579 | GIDGRTVSRIEGQGVEEFLAGLRESLKSGEFWPVPVKERMIPKAN | >DS_Ca.ac.I1/C |
| | GKLRRLGIPTVADRVVQAALKLVLEPIFEVDFEPCSYGFRPNRRA | P001700/4538431 |
| | HDAIAEIHHYASRGYEWVLEGDIEACFDNIDHTALMGRVRERVGD | Catenulispora |
| | KRVLRLIKAFLKSGIFSEGRAVRDTRTGTPQGGILSPLLANVALA | acidiphila/ |
| | VLDEHFAQVWQETGRTWAARDWHRRRGGATFKLVRYADDFVILAY | Unclassified |
| | GSRQHVEDLTADVAQVLSTVGLRLSPTKTAVAHIDEGFDFLGFRI | |
| 8580 | GVDGVAPRSLLHGQAVEVLTMIRRQVKTGEFRPLPVRERRIPKSN | >DS_gi\|2609054 |
| | GKTRSLGIPTLADRVVQASLKLVLEPIFEADFYPSSYGFRPRRRA | 81\|ref\|ZP_05913 |
| | QDAIAEIHKFTSRPLNYEWVFEADITACFDEIDHTGLIQRLRGRI | 803.1\|Brevibacterium |
| | TDKRVLALVRRFLKAGILSEDGVNRNTHTGTPQGGILSPLLANIA | linens |
| | LSGLDDHFQKKWESLGPSWTRAKLRRRGIPVMKLIRYADDFVVLV | BL2 |
| | HGSVEHVEALWHEVAEVLAPMGLRLSVEKTKVTHIDEGFDFLGWR | |
| | I | |
| 8581 | GADGITFAQIETEGRERWLENVRQELTAGDYRPQPLLRVWIPKSN | >DS_gi\|3549613 |
| | GGRRPLSIPTVKDRTVMTAAMLVIGAIFEADLLENQYGFRPKVDA | 71\|dbj\|BAL1405 |
| | KMAVRRVFWHIRDHRRSEIVDADLRDYFTSIPHAPLMKCLTRRIA | 0.1\|Bradyrhizobium |
| | DGRLLSMIKGWLTVAVIEKDGRRITRTAEARTKKRGTPQGSPLSP | japonicum |
| | LLANLYFRRFLLAWRHGHQDQLDAHIVNYADDFVICCRPGSSETA | USDA |
| | MARMQTLMNRLGLEVNDTKTRLARVPESVTFLGYTI | |
| 8582 | GVDGVTIEEIMKTDQGVAGFLEGIENSLRRKTYRPEAVQRVYIEK | >DS_So.us.I2/C |
| | ENGKLRPLGIPTVRDRVVQMATLLILEPIFEADFLDCSYGFRPGR | P000473/ |
| | SAHQALEEIRGHVEAGYQAVYDADLKGYFDSIPHTQLLACVRMRV | 3231872 . . . |
| | VDRSVLKLIRMWLEAPVVEREEGGGGSKWSRPEKGTPQGGVASPL | 3233814/ |
| | LANLYLHWFDALFYGPEGPGGKADAKLVRYADDFVVMAKQMGTET | Candidatus |
| | IEFIESRLEGKFQLEINREKTRVVDLREEGASLDFLSHTF | Solibacterusitatus/ |
| | | Bacterial |
| | | F |
| 8583 | FGVDGVSIESIEVRADGISGYLDEIQESLRTKNYKPSPVRRVYIT | >DS_Ge.ur.I1/C |
| | KPNGKLRPLGIPCVRDRIVQAAVLLILEPIFEVDFLDCSHGFRPK | P000698/ |
| | RRPHGALDQVGNNLQLGRQEVYDADLSSYFDSIPHEHLIVELERR | 1525569 . . . |
| | IADRSVLKLIRQWLHSPVREEDGSISRPKQGTPQGGVISPLLANI | 1527641/Geobacter |
| | YLHRLDRAFHEEADSPYHFARARMVRFADDFVVMARHMGNRITGW | uraniireducens/ |
| | LEEKLETDLGLSINRDKTGIVRMNKKESLNFLGFTL | Bacterial |
| 8584 | GIDDVTIDEFERNLEQNLNEIQRLLRQDRYVPKPVKRVYIPKPDG | >DS_UA.14/AY |
| | KQRPLGIPTIRDRVVQQALKNVIEPIFEAEFLDSSFGYRPGKSAK | 714820/20258 . . |
| | QAIEQIETVRDEGHEWVVDADIKAFFDTVNHEKLIDAVAERISDG | 22206/uncultured |
| | RVLGLIRAFLEADIMEQGQGRAKNVVGTPQGGVISPLLANIYLHY | archaeon/ |
| | FDERMALGFEVVRYADDVLVLCGSEEEAEEAISHVKEILEELELT | Unclassified |
| | LHPQKTKIKNFSEGVDFLGFTV | |
| 8585 | GVDNQTLDDIREEGIEQLLEQIQHELKTGTYRASCVRRVFIPKSS | >AC_fig\|115547. |
| | GKLRPLGIPTVKDRIVQQAVKLIIEPIFEADFLEFSYGYRPNRSA | 10.peg. 1390\| |
| | KDASLEIYKWLNYGLTNIVDVDIEGFFDHIDHELLLKFVKERVTD | [uncultured |
| | GYILSLIKQWLKAGIVYGKSVTNPTEGTPQGVSFLR | archaeon\| |
| | | 11554710] |
| 8586 | GVDGETIEDIENRGVDQFLTEIQQQLRMKTYRIPKVKRVFIPKGD | >AC_fig\|115547. |
| | GKLRPLGIPTIRDRVVQQAVKSIIEPIFEADFKDCSFGYRPGRSA | 10.peg.767\| |
| | MQASEKIRHLLNLGYTNIVDMDIKGFFDHIDHEKMVFSVMKRITD | [uncultured |
| | PYVIKLIREWLRAGIVFQGNTSYPEQGTPQGGVISPLLANIYLNE | archaeon\| |
| | LDSLWTRRGMESPLKHSAHLVRYADDLLALTNKDPQAVAETLERI | 115547.10] |
| | ISLLGLEPNREKSSVITAEDGFDFLGFHFI | |
| 8587 | GVDGERFEDVEAYGVERWIGELAETLRKKMYQPQAVKRVYIPKPG | >DS_gi\|3442004 |
| | GKMRPLGIPTLRDRVVQTATMMVIEPIFEADLQPEQYAYRAGRNA | 32\|ref\|YP_00478 |
| | LTAVREVHSLLKTGHKQVVDADLSSYFDTIPHAELMKSVARRIVD | 4758.1\|Acidithiobacillus |
| | RHLLHLIKMWLDAPVEEGDGRGNMQRTTVNRDQGRGTPQGAPISP | ferrivorans |
| | LLSSLYMRRFILGWKQRGYEERFGSRIVCYADDLVICCRWQAEQA | |
| | MAAMQDMMGRLKLTVNAEKTRICRVPEAYFDFLGYTF | |
| 8588 | GVDRQDFEDVEAYGVRRWLEELALALKEESYRPDPIRRVFIPKAN | >DS_D.a.I1/CP0 |
| | GKLRPLGISTLHDRVCMTAAMLVLEPIFEADLPDEQYAYRPGRNA | 00089/759875 . . . |
| | QQAAEEVKNRLYLGQTDVVDADLSDYFGSIPHSELMKSLARRIVD | 761862/ |
| | RRVLHLIKMWLECAVEETDQRGRKKRTTEAKDQGRGIPQGSPISP | Dechloromonas |
| | LLSNLYMRRFVLAWKKLGLERSLGSRIVTYADDLVILCKCGKAEE | aromatica/ |
| | ALQWMRTIMGKLKLTVNEEKTRICQVPAGTFDFLGYSF | BacterialF |
| 8589 | GVDRQDFAEVEAYGVQKWLGELALALRLETYRPDSIRRVFIPKAN | >DS_gi\|2961637 |
| | GKLRPLGISTLRDRVCMTAAMLVLEPIFEADLPPEQYAYRPGRNA | 94\|ref\|ZP_06846 |
| | QQAVIEVEERLHRGQTDVVDADLADYFGSIPHAEMMLSLARRIVD | 488.1\|Burkholderia |
| | RRVLHLIKMWLECPVEETDDRGRQKRTTEARDSRRGIPQGSPISP | sp. |
| | LLANVYMRRFVLAWKKLGLQRSLGSRIVTYADDLVILCKKGKAEE | |
| | ALLNLRQIMGKLKLTVNEEKTRICKVPEGEFDFLGFTF | |
| 8590 | GVDGITFEQIDASGLEAWLAGLRDELVTKTYRPDPVRRVMIPKPG | >DS_gi\|3549604 |
| | GGERPLGIPTIRDRVVQAAAKIVLEPIFEADFEDGAYGYRPRRNA | 51\|dbj\|BAL1313 |
| | VDAVKEVHRLMCRGYTDVVDADLSKYFDTIPHSDLLKSVARRIVD | 0.1\|Bradyrhizobium |
| | RNVLRLIKLWLRVPVEERDSNGKRRMSGGKSNKCGTPQGGVISPL | japonicum |
| | LSVIYMNRFLKHWRLSGRCEAFHGQIISYADDFVILSRGHAEDAL | USDA |
| | TWTKAVMTKLGLTLNETKTSVKNARLESFDFLGYTL | |
| 8591 | GVDGMTFGQIEGAGVDAWLAGLREDLVSKTYQPDPVRRVMIPKPG | >DS_Ni.ha.I1/C |
| | GGERPLGIPTIRDRVVQAAAKIVLEPIFEAGFEDSAYGYRPRRSA | P000320/75444 . . . |
| | IDAVKETHRLLCRGYTDVVDADLSKYFDTIPHADLLRSVARRVLD | 77354/Nitrobacter |
| | RNVLRLIKLWLQVPVEERDGDGKRHMSGGKSSTRGTPQGGVASPL | hamburgensis/ |
| | LSVIYMNRFLKHWRLTGRGEVFHAHVISYADDFVILSRGHAEEAL | BacterialF |
| | TWTRAVMTKLGLTLNEAKTSVKNARREGFDFLGYTL | |
| 8592 | GIDDFTIEEIEAYGVQKFLDEIEDQLRNKKYQPKAVKRVYIPKAN | >DS_S.ag.I2/AE |
| | GKKRPLGIPTVRDRVVQTAVKIVIEPIFEADFQEFSYGFRPKRSA | 014217/10188 . . . |
| | NQAIREIYKYLNYGCEWVIDADLKGYFDTIPHDKLLLLVKERVTD | 12210/ |
| | KSIIKLLSLWLEAGIMEDNQVRSNILGTPQGGVISPLLANIYLNA | Streptococcus |
| | LDRYWKNNRLEGRGHDAHLIRYADDFVILCSNNPKKYYQYAKQRI | agalactiae/ |
| | DKLGLTLNEEKTRIVHATEGFDFLGYTL | Unclassified |
| 8593 | GIDGVTFEAVEEKEGVSAFIAELEDALRNKTYQPDPVKRVMIPKS | >DS_gi\|3224179 |
| | DGSQRPLGIPTIRDRVAQMAVKLVIEPIFEADFCESSYGFRPKRS | 44\|ref\|YP_00419 |
| | AHDAVDDVAYSMNTGYTEVIDADLSKYFDTIPHANLMAVIAERIC | 7167.1\|Geobacter |
| | DGAILHLIQMWLKAPIMEVDKDGTKRNIGGGKGNRKGTPQGGVIS | sp. |
| | PLLANLYLHILDRIWERGNLQQRLGARIVRYADDIVILCRRAKAD | |
| | KAMATLRYVLERLGLSLNEAKTTTVNAYKDKFDFLGFTI | |
| 8594 | GIDGVTFTAIEAGIGKDAYVAALREELEQKTYRADGVRRVWIPKP | >DS_gi\|3458701 |
| | DGSERPLGIPTIRDRIVQMAFKLVVEPIFEADFCEHSYGFRPQRS | 11\|ref\| |
| | AHDAIDAIAEALLRGHTQVIDADLSKYFDTIPHAKLMGVIAERLV | WP_139058630.1 |
| | DGPVLGLIRQWLKAPVIEEDERGQHRPTGGKGNRRGTPQGGVASP | \|Thiorhodococcus |
| | LLANLYLHLLDRIWVRHDLERRLGARLVRYADDAVILCRHSTEKP | drewsii |
| | MAVFTAVLEKLDLTLNVQKTHVVDARADGFEFLGFRI | |
| 8595 | GSDGVSFEAIEQGEGVEGFLKGLAEELREKRYRAQPVRRAMIPKG | >DS_gi\|3505548 |
| | DGRERPLGIPTIRDRVVQMAVKLVIEPIFEADFTPHSYGFRPQRS | 47\|ref\| |
| | AHDAIDDIANALWAGHTHVIDADLSSYFDTIPHANLMTVVAERMT | ZP_08923 |
| | DGAILALLKQWLKAPIIGVDDQGKRRTVGGGKANRVGTPQGGVIS | 874.1\| |
| | PLLSNLYLHLLDRIWDRHRLKDKLGAHIVRYADDFVVLCKQGVEE | Thiocystis |
| | PLKVVRHVTDRLGLTLNETKTHVVDAKETGFHFLGFTL | violascens |
| | | DSM |
| 8596 | QCDTSLYQTWLSSIAQDTHSPLKKAELENLLEALHSLSYIPSVAH | >PF_WP_03688 |
| | AIHIPKSDGSYRTLSIPSPIDLYLQRNLINVLYPIIDKTNSPQSY | 5018.1 |
| | AYRKGKGALEAIKQVELLKRKLGKKYYVVRCDIDNFFDSIPIEQL | [Porphyromonas |
| | MGMFQNITRDPLLSRMVRLWIKSGVVDNKSHFHPHLQGLPQGSPL | gingivicanis] |
| | SPLLSNFYLTDTDRYISNNITEYFIRYADDILLFIPEHSDPLSSL | |
| | QALSNHLKNQKKLSLNKDFIVTEINSEFSFLGISF | |
| 8597 | VNDALYRKWLSSLAADRDLPMAEAERQDLLEALRVCSYIPQPYHS | >PF_WP_03944 |
| | VNIPKGDGSYRQLHIPSAVDLHLQRSLAGILYPITESLSIAQSYA | 3024.1 |
| | YRKGKGAVAAIRKVQHLLDSLDENYTVVRCDIDNFFDSIPVPSLL | [Porphyromonas |
| | QKVLRTTEDPLLTRMLSLWMKSGVVDRTQQYTPASSGIPQGSPLA | gulae] |
| | PLLSNLYLEDTDRYIAGHITTEFIRYADDLLLFLPERADPLKALQ | |
| | DLSEHLKYRKGLKLNRDFVVSSIKSSFSFLGITF | |
| 8598 | RKWLSSLAADRDLPMAEAERQDLLEALRICSYIPQPYHSVNIPKG | >W_[ |
| | DGSYRQLHIPSAVDLHLQRSLAGILYPITESLSIAQSYAYRKGKG | Porphyromonas_ |
| | AVAAVRRVQHLLDSLDENHTVVRCDIDNFFDSIPVPSLLQKVQRT | gingivalis] |
| | TEDPFLTRMLSLWMKSGVVDRKQQYARASSGIPQGSPLAPLLSNL | 34541577 |
| | YLEDTDRYIAGHITTEFIRYADDLLLFLPEKVDPLNALQDLSEHL | |
| | KYRKGLKLNRDFVVSSIKSSFSFLGITF | |
| 8599 | DTLYRKWLSSLAADRDLPMAETERQDLLEALRICSYIPQPYHSVN | >PF_WP_01381 |
| | IPKGDGSYRQLHIPSAVDLHLQRSLAGILYPITESLSIAQSYAYR | 5267.1 |
| | KGKGAVAAVRRVQHLLDSLDENYTVVRCDIDNFFDSIPVPSLLQK | [Porphyromonas |
| | VQRTTEDPLLTRMLSLWMKSGVVDRKQQYAPASSGIPQGSPLALL | gingivalis] |
| | LSNLYLEDTDRYIAGHITTEFIRYADDLLLFLPEKVDPLNALQDL | |
| | SEHLKYRKGLKLNRDFVVSSIKSSFSFLGITF | |
| 8600 | AIEEVLERHELEPVVDDKGRIMQVDALTELIYTQLSTGVYAPKPT | >PF_WP_01967 |
| | RAIFVPKPKGGKRCIEELEQVDMMVHRLVFNSIAKTIESYQSPLS | 2870.1 |
| | LGYRKGYSRQMARDKVQALIDSGFGWVVEADIESFFDNVPFERLW | [Psychrobacter |
| | QRLATILPQRELQTIALIKKLMQVGYTVSNASGTVVKEHLRFKGL | lutiphocae] |
| | MQGSPLSPVLANLYLAMLDEQINAEHFAFVRYADDVLMFCRSEAD | |
| | ANTTLAWLDQHLSELGLNLSLSKTAITAVNNGFEFLGYRF | |
| 8601 | GAAASRLDRDNDFSASLERDYGSIGNAVFAMRDHILNGEFQCSPA | >PF_WP_02718 |
| | APFEINKPLGGRRTLGTFSSEDSLAQKLLHRLLSPVLDRMFEHSS | 0402.1 |
| | VGFRKGRSREDAKRMIQQAIRQGCRYVFESDIDSFFDDIDRSTML | [Desulfovibrio |
| | RKLRGVLPQADKMTFRALESCINAGLENEVDSTKGLVQGSSLSPL | bastinii] |
| | LSNLYLDSVDERMDEHGYRFIRYADDFVVLAHSEDEWRKACEDMQ | |
| | DSLEPLGLQLKEGKTHISCIDPGFKFLGIEL | |
| 8602 | GAAASKINRDSDLSSELERDYGSIEKAVFEMRDRILMGEFTCSAA | >W_[Desulfovibrio_ |
| | VPFEMHKPYGGSRIIGTCPPEDTLTQKLLHQLLSPVMDRMFEHSS | hydrothermalis]_ |
| | VGFRKGRSREDAKRMIRQAIREGCRYVFESDIDSFFDEIDRPTML | (2)436839745 |
| | RKLQDALPQADHMTFKALKSCVNAGLVDEDRQDAKGLVQGSSLSP | |
| | LLSNLYLDGVDERMEELGYRFIRYADDFVVLARSKEECRKAYEDM | |
| | RLTLAPLGLSLKEQKTRISNIDPGFRFLGIDL | |
| 8603 | DLAVRLSNTPQAPDLHELAVELAQNLREGAAPLPFQAIRVPRSDG | >PF_KFB71594.1 |
| | RLRQFETPAARDLVILNHLTRLLSEPFDRLFSVHSIGYRKGHSRE | [Candidatus |
| | DAVERVRAAIAEGCTHVLESDISDFFPSVDLKRLLARLDDVLPRR | Accumulibacter |
| | DVRLRQTLAAYLGAGWRYGEGSVQARNRGLPLGSPLSPLLANLYL | sp. BA91] |
| | DSFDSQLGATVPGVRLIRYADDFIILTESEAAARALLDTARDAAA | |
| | ALGLALNLEKTAIRPLSDGFDFLGIRF | |
| 8604 | YTPAPNTAFLIKKKSGVDRMVEQIALKDLILQQYLLKTIGNEFER | >PF_KFZ44108.1 |
| | IFEPESIGFRKGISRQRAVEMVQAALKAGYQFIIESDVDDFFPSV | [Smithella |
| | DLKILTGLLDRYLPQEDHRIKELLTKTIHNGYVLNGQYHERVRGV | sp. D17] |
| | AQGSPLSPMLANLYLDYFDETIKGWPVRLIRYADDFIILTRTKEE | |
| | AEEYLSRTESCLSEIGLKIKKEKTGIKHIREGFRFLGIKF | |
| 8605 | ALQALSETEKYPFDENQYAENLFQLIVSNGYLPTPHIAFTIKKKS | >PF_KKO19838.1 |
| | GVDRVVEQLSFRDLIVQQYLLKVISTVFDRFFEAESIGFRKGVSR | [Candidatus |
| | QRSIGMIQSAIAEGYQCVIESDIEDFFPSVDLDILEHLLDCSIPQ | Brocadiafulgida] |
| | NDVCLKNILLKLIRNGFILNGTYYERRKGLAQGGPLSPILANLYL | |
| | DSFDEQIKRWGLASHDEDAGTDHAKGGAGNKTAGGNASRGVKLIR | |
| | YADDFIILTRTKEEAEGVLSDTESYLSTLGLKIKKEKTAIRSLRD | |
| | GFHFLGIRF | |
| 8606 | VEQIPFRDLIVQQYLLKIISAPFDRFFEAESIGFRRGVSRQRSIE | >PF_WP_05256 |
| | IIQAAIAEGYQYVIESDIEDFFPSVDLNILAHLLDSYIPQNDSCL | 5451.1 |
| | KKILLKFIKNGYILNGVYHERVKGLAQGSPLSPILANLYLDSFDE | [Candidatus |
| | QIKQWGLLSPDEHGETASDSKDTPSRAAPAHTPRGVKLVRYADDF | Brocadiasinica] |
| | IILTKTKKDAEDVLSETEAYLSKLGLKIKKEKTAIRSMKDGFQFL | |
| | GIRF | |
| 8607 | GLLQRQTRLIAGDLDRFLAELSASLRGGTYLPAPLLRADIPKRAP | >PF_WP_05358 |
| | GQTRVLHIPTIRDRVVERAVVNAVAHDADRIMSPCSFAYRTGIGT | 7381.1 |
| | DDAVHHLATLRDDGYRHVLRTDVEDYFPNLDVEDALTVLAPVVGC | [Actinomyces sp. |
| | PRTIDLIRLIARPRRARGERRTRSRGIAQGSCLSPLLANLVLNDV | oraltaxon |
| | DHALNDAGYGYARFADDIVVCAPARDDLLAARELLGSLVAAHGLN | 414] |
| | LNEEKTAMTTFDEGFCFLGVDF | |
| 8608 | GVLQRQSKRIIENADEFLNQLSALLRNGTYEPEPLNRVDIPKGEH | >PF_ENO18597.1 |
| | GKTRTLNIPTIHDRIVERAIVDTIAFTADLVQSSCSFAYRTGIGV | [Actinomyces |
| | DDAVHHVATLREEGYQYVLRTDIEDFFPHVNLEHALEALPESLQE | cardiffensis |
| | RDLLALLRIVALPRRAHGQRRARSRGVAQGSTLSPLLANLSLTRF | F0333] |
| | DHDICDAGYGYARFADDIVVCSPREQDILDAIELLSDLAAAHGLK | |
| | LNQDKTIMTTFDEGFCYLGVDF | |
| 8609 | RADIADCFEQIPRWPVVTRVKELVPDAEPCLLIQHLIARDATGPA | >PF_WP_02038 |
| | ARRVWSGRRRSRGLYQGSALSPALADLYLGAFGKAMLWAGRQVLR | 0191.1 |
| | YADDFAIPAGSRTEAESALTTAEDVPAEWGPELNGAKSRIVSFDE | [Nocardiopsis |
| | GVDFLGRTV | potens] |
| 8610 | GVKSTAVQEFEKGALRRLLDISEQLREGTYAPEPVTAFEVPKPSG | >PF |
| | EARLLGIGTVGDRVVERAVLAVIEPCIDPVLLPWSFAYRKGLGVP | WP_083934465.1 |
| | DAVQALAEARESGSTWVLRADFADCFETIPRWPVITRLHELVPDA | [Nocardiopsis |
| | ELCLLVQHFIQRKSRGPGARRLRPGSGRGLHQGSALSPLLSNLYL | baichengensis] |
| | DSFDRALLQRGRQVLRYGDDFAVPSESRHAAEQALAQATEAAREW | |
| | GLELNAAKSQIVSFDEGVRFLGRTV | |
| 8611 | GQPDSEVDAFEANAARNLDELGTVLAAGEWQASPVRRVDLPKPSG | >PF_WP_05291 |
| | GVRVLGVPRLVDRIVERALLRVLDPVIDPLLLPWSFAYRRGLGAR | 4180.1 |
| | DALAALAEARDSGMTWVARSDIRDCFPSIPQWEVLRRLREVVDDE | [Frankia |
| | RIIHLVGVLLDRPVAGGRTDPKNRGLGLHQGSALSPLLSNLYLNA | sp. BMG5.1] |
| | FDRAMLRAGFRVIRYSDDFAIPTTGRVAAEQALVSASTELEDLRL | |
| | EINSGKSHVVSFDEGVRFLGEVT | |
| 8612 | TRVSIPKPDGGIRSLAIGAIEDRIVERAVLDVLDPVVDPTLSPWS | >PF_KXK58998.1 |
| | FAYRRGLGVRDAVRALAEARESGLAFVVRCDIDDCFDSIPRWPLL | [Micromono |
| | RRLRELVSDAELVALVERLVGRPVTGERASGGRGLHQGGSLSPLL | sporarosaria] |
| | ANLYLDTFDRALMRHGHRVVRYGDDIAISVPDRPTGLRVLDLADA | |
| | EAEALSLRLNTDDRQVIAFDEGVPFCGQVV | |
| 8613 | GRVPVSVRRFERGVAASLVRLSGELSSGRYQPSRVSEVSLRTGSG | >PF_WP_052104813.1 |
| | SERVLRIGAVVDRVVERSLLNALTPVIDPLLSPFAFGFRRGLGVK | [Cellulomonas |
| | DAVAALARARDEGSTHVLRSDIAAAFDSVPRARAVQALSRLVPDR | bogoriensis] |
| | RVCDVVASLLARLDDYGLEGVGIAQGSAVSPLLLNLYLLPFDEAL | |
| | MANGFTPLRYADDIAVPAMSESQAQSAAQDVAHQLECLGLACSAP | |
| | KTSIRSFDEGVHFLGVTL | |
| 8614 | GNLAPSILKFQEDAEEKILRLSEALLDGSYKPYQFTEVDIETNGK | >PF_WP_00606 |
| | ERTLHIPAVQDRIVARAILATTTSRIDPLLGASAFGYRPGLGVAD | 3846.1 |
| | AVQAVVDAREAGLKWVLRTDVDDCFPSLSPDIAFDRFTQAVHDTD | [Corynebacterium |
| | ITDVVEQLLGRTVGNGKMRGTTLPGLPLGCPLSPVLMNLVLVDLD | durum] |
| | DALNAAGFTVVRYADDIVVVGESKEELEDAARFCQRILRSFNMQL | |
| | GDDKTDIMTFDDGFAFLGEDF | |
| 8615 | AVLVPGPTRPDLPGQGVKVDQWSYTTLVDLTEGLRWVCRCDIDNC | >PF_ACV77640 |
| | FPSIPKDRLRRKLTALFQGDPTLLGILTRLLARPAGGSPAEALPG | .1 |
| | LPQGSPLSPLWANLILADFDDAVARTGFPLVRYSDDMVIAAADRA | [Nakamurella |
| | EAWEAMRVAHDAAAGIEMSLGADKSAVMSFDEGFTFLGEDF | multipartite |
| | | DSM44233] |
| 8616 | PDRIVARAILDTATPFVDPELGHCAFAYRPGLGVADAVQAIARQR | >PF |
| | EEGLGWVLRTDIDECFPTLPVDLAHRRLAALVDDDDLASVLTALS | WP_211223266.1 |
| | ARPYRTATRALRAVTGLPQGCPLSPVLANLVLVDVDRALLDRGYA | [Propionicicella |
| | PVRYGDDIAIPCANEDDAWEAARVTSEAAERLDMSLGSDKTHAMS | superfundia] |
| | FTEGFVFLGEEF | |
| 8617 | DQLSAGVRTFGDEADQRLAGLAEQLAGGMYLPGVLTELVMVTEDG | >PF_WP_05239 |
| | GQRVLRVPAVRDRVVERALLSVLSPRLDPLLGPASFGFRPGLGVV | 6493.1 |
| | DAVQALARLRDEGFGWVLRTDLHDCFPSVDLRRVRRLLEVLTSDG | [Kutzneria |
| | DLLGVLDLLLARAARRPGEQTLRPAHGLPQGSSLSPLLANLVLED | sp. 744] |
| | FDDRMRHAGFPLVRYADDIAVLASSEREAWEAARVASAAAKEIGM | |
| | TLGADKTEIMSFDGGFCFLGEDF | |
| 8618 | GVERFAEDPKAELDELGEQLRTGTYRPRDLTEVVIDDGGGSRTLH | >W_[Microlunatus |
| | IPAVRDRVVERSLLNVVTPWVDPVLGFTSYAYRPGLGVADAVQAL | phosphovorus] |
| | VTLRSEGLGWVLRTDVDDCFPSVPVDHARRLLGALVPDADLLAIV | 336116789 |
| | DLLLARAAVRPGRGRGVMRGLAQGCALSPLLTNLVLTALDDALLD | |
| | EGFAVLRYADDICVATETRDDAWEAARIATAALEVLGMELGADKT | |
| | EVMSFDEGFSFLGEDF | |
| 8619 | ALDQAASKWDLGGGEVQSAARDLVKGTYQPQPCFRLDIPKSNGDR | >W_[Pirellula |
| | RQLAIPSRLDRVLQRSILDVIAPALELFFEESSFAYRRGLGRHTA | staleyi] |
| | ARHLSQAFTDGYRWALHADFFDFFDTIDHKLLRRRLAAYLADPSL | 283778924 |
| | VEVIMRWVETGAPHPDHGIPTGAPLSPILANLFLDQFDEAMHSVG | |
| | RRLVRYADDFVVLFRDQSEAQAVISEVRQAAESLRLELNRDKTHT | |
| | LHLATSFDFLGLHF | |
| 8620 | IEKKPTIDKLTERTHSQINSALGQIIKQDYNAPAMQGFTIPKKDG | >PF_WP_03813 |
| | SERLLAVSPLYDRVLQKAAAIILTPGLDALMAQGSYGYRKGLSRQ | 7810.1 |
| | QVRYEIQNAYRQGYHWVYESDIEDFFDAVNRKQLLNRLQSLFGKD | [Thiomicrospira |
| | PIWKQLEDWLGQEIHYQETIVERTPHTGLPQGSPLSPVLANFVLD | sp. MilosT1] |
| | DFDSDMELHGFKMIRFADDFIILCKSRHEAELAALGVQHSLKQVS | |
| | LDINRDKTHIVELSQGFRFLGYLF | |
| 8621 | DTDEEHHDAIDELLTKLYVSRERIFKREFTPSQLHSVEIEKPEGG | >W_[Marinomonas |
| | TRLLSVPNWHDRTLQKAVTECLGNTLEHIWMKHSYGYRKGHSRLQ | mediterranea] |
| | ARDQINQYIQQGYEWVLESDIESFFDSVNWLNLEQRLKLLLPNEP | 32 |
| | LVPLLMQWVSAAKQTEDEQTLARHNGLPQGAPISPILANLLLDDL | 6793969 |
| | DQDMIAKGHQIVRYADDFVLLFKSKAAAESALDDIITALKEHHLA | |
| | INLEKTRIVEASQGFRYLGYLF | |
| 8622 | SFNITATEFRTTLYQQLAAIRACRYHPHPLVPVTIAKKDGTDRFL | >PF_WP_02888 |
| | AVPPVGDRALQRVVTAQLSAELDPLFIQHSFGYRKGYSRQGARDA | 3449.1 |
| | INQAIRAGYGWILESDIDSFFDSVAWSQMATRLRLFMGQDPLVDL | [Teredinibacter |
| | IMQWLQTPVQETPAASAPAPRCAGLPQGAPISPLLANLLLDDFDQ | turnerae] |
| | DMIVQGMKLVRFADDFVLLFKHQQQAQQALPRVVQSLAEHGLALK | |
| | PEKTRIVSAQQGFRYLGYLF | |
| 8623 | SLDSQFSEQERNQYKNKVIGLSHTILAGDYKAPVLTQVEIDKSDG | >PF_WP_03818 |
| | GVRTLSIPPLADRILQKAIARPLAVSLDGLWKTHSYGYRKDLSRH | 8758.1 |
| | DAKFAINQAIQQGYEWVLESDVDSFFDNVDWRNLQTRLKLLLPND | [Vibrio |
| | FLVDVIMAWVKAPVKTPSGQILERTQGLPQGSPLSPLLANLVLDD | sinaloensis] |
| | FDADMLALDYKLIRYADDFVLLFKKQSEAQMALDHVIASLNEHGL | |
| | NIKAKKTQIVHANKGFRYLGFWF | |
| 8624 | SLDNPLSEQQQREVLQSVMTGSECLIHQRYPVPTLQQVEIEKEEG | >PF_WP_05504 |
| | GTRTLSIPPLIDRILQKAVARPLAASLEGLWKSHSYGYRSGLSRH | 3549.1 |
| | DAKLAINQAIQNGYEWILESDVESFFDNVDWHNLETRLTLLLPND | [Vibrio |
| | ALVDTIMAWVKAPIKTVTGEYQQRQQGLPQGSPLSPLLANLILDD | metoecus] |
| | FDADMLALDYQLVRYADDFVLLFKTEQQAQAALHRVIDSLNEHGL | |
| | KIKAQKTHIVHAKTGFRYLGFWF | |
| 8625 | SETQKQHTLTQLRQQCAQLLEGTFTAPTLQQVDIDKDDGGTRTLS | >PF_WP_04787 |
| | IPPWQDRVLQKAVASLLNEAFDPLWKHQSYGYRKGRSRFNAKDAI | 5592.1 |
| | NDAIRQGYEWALESDVDSFFDSVCWTNLAARLHLLFPSDPLVPVI | [Photobacterium |
| | MNWVKAPIRTPDGDEIPRTQGLPQGSPLSPLLANLILDDFDGDML | aphoticum] |
| | ALDYQLVRYADDFVLLFTSQQQAQQALPHVIASLNEHGLTLKARK | |
| | THIVEAKKGFRYLGFLF | |
| 8626 | ECDLSQYECNEETDAEGDQAELPPTLLKRANALAQGRYDVPPLRG | >PF_WP_01960 |
| | VIIPKTDGEWRALAVAPFFDAVLQRAVAQILAPSLDRVMDNRSYG | 6016.1 |
| | YRRGRSRLDAKEQIQLAYRNGARWVLEADIEDFFDSVAFSLVAQR | [Teredinibacter |
| | LRALFHQDPINEAILAWLSAPVDYDGLRLQRKAGLPQGSPLSPVL | turnerae] |
| | ANLLLDDFDSDMRKAGFNCLRFADDFVVVCQSREEAERAWQRAAS | |
| | SLNEHGLFLAENKTRVISFERGFRFLGYLF | |
| 8627 | GVEWIDADEQEPDAQDGAEAEADELAAPIEDLTRAIGHLQEGKYR | >PF_ESQ17084.1 |
| | VPELRGYLLPKRDGGLRPLAVPPLRDRVLQRAVQQTLGRGIEPLF | [uncultured |
| | SSGSHGYRPGHSRITAADAIRAAWAQGYRWVYESDVRDFFDSVDL | Thiohalocapsa |
| | QRLRERLEAIYGDDPVVAAVLGWMRAPVRFRGERIERRNGLPQGS | sp. PBPSB1] |
| | PLSPLMANLMLDDFDSDMQAAGFRLIRFADDFIVLCKDPEEARRA | |
| | GEAARASLAEQGLALHPDKTRITAMEDGFRYLGYLF | |
| 8628 | PIDPDDPESMPDPEAEEALADRLEAIGERLRAMRYQAPALKGVVI | >PF_ESQ08042.1 |
| | RDPDGDLRALAIPPFWDRVAQRAVNDCITPACDLLMSEASHGYRR | [uncultured |
| | GRSRHTASLDINRAWQDGYRWVYEADIEDFFDSVDWDKLRLRLEA | Thiohalocapsa |
| | LYRDDPVIDLILAWMAAVVDYQGFSVQRSMGLPQGAPLSPTMANL | sp. PBPSB1] |
| | MLDDLDNDLEQAGFRLVRYADDFVVLCRDRAQAEAAGQEVRRSLA | |
| | ELGLQLNDAKSRVVSFQQGFRFLGFVF | |
| 8629 | GGELWDTEYPEAPDPDEEEELADRLERLGKRLLEGDYRPPALRGV | >PF_CRI67871. |
| | VYRDPDGDLRGLAIPPFWDRVAQRALVERIAPALEGVFSAASHGY | 1 [Thiocapsa |
| | RPGLSRHTASSAIQRAWREGYRWVYEADIEGFFDNLDWQRLAERL | sp. KS1] |
| | RALYRDDPAVDLLLAWMAAPVDYQGMRIERSRGLPQGAPLSPVLA | |
| | NLMLDDLDSDLEHAGFRLVRYADDFVVLCKDQERARAAGEAVRRS | |
| | LAELGLILNESKSRSVSFEQGFRYLGFLF | |
| 8630 | GQSIAAFAAKGAAAIARLSGLLRNGNYAPRPLRLHEIPKPDGGTR | >W_[Rhodobacter |
| | RLAIPAVSDRIVQTAVAAALTPGSSRCFPPTATATAPAVRWRWRW | capsulatus] |
| | TGSRPCGGWATPGWSRPISKRPLTGSRMTRCSRRSIP | 294676824 |
| 8631 | ESKKLPKKLHQSLPHEDFDQLYEQLHQGNYQTGLLTPRLLEKPGQ | >PF_WP_00746 |
| | KRRLLLLPPFIDKVAHKCLSRWLATSLDTLYSANSYGYRKGYSRL | 9744.1 |
| | TAKDRISYLLSQGYKWVVDADIKAFFASIDRQQVAARLQALYGDD | [Photobacterium |
| | TLWPILDKMLNAAIDPNSELPLELSISGLNLGNSLSPILANLMLD | marinum] |
| | HFDDVIREHKLELVRYADDFLILCREQQQANHAKDFVEQLLHSQA | |
| | LSLNPRKTRVTHVNKGFRFLGYLF | |
| 8632 | DEQQALEQQRSDLPEMLQQVLHHDYWPAPLTPWLMQEQGRKERLI | >PF_KUI97421. |
| | LLAEFNDKVLHKTISLWLGASLDQLYSKTSYGYRKGYSRLSAKDR | 1_(2) |
| | IINRIRAGYVYAVDADIRDFFPSVEQSRVLNRLSALYGADPLWTL | [Vibrio |
| | VERFLSAPIRRQHLPAGYEDYERTGLDLGNSLSPVLANLMLDHLD | sp. |
| | AVMESLGYELIRYADDFLVLTKSRDKAQQALHMIEEILTAQGFAL | MEBiC08052] |
| | NHEKTRIRHFSEGIHFLGYLF | |
| 8633 | TDEHKAWEQSRENLPDTLYRCWHLDYWPELLHPRLLQQAGKKERL | >PF_WP_02830 |
| | LLLPPFPDKVIHKTISRWLSDSLDQLYSKSSYGYRKGYSRLGAKD | 2067.1 |
| | RIIHLVRKGYKYALDADITDFFPSVNTNRILGRLAALYGQDPLWQ | [Oceanospirillum |
| | LLERFLHCQIDRQNLPAGYEHHVNQGLNLGTSLSPVLANLMLDHL | beijerinckii] |
| | DSVLNNMDYELVRYADDFLVLCKHKQQAEDARVLIEQLLKQHDLQ | |
| | LNAEKTKVRSFASGIYFLGYLF | |
| 8634 | GVDGITTDLFVGVANEQLAQMHRQLRREVYEASPAKGFYVPKKNG | >PF_KPQ33062. |
| | GQRLIALSTVRDRILQRYLLQSIYPRLEKAFTDSTFAYRPGLSIY | 1 [Phormidesmis |
| | GAVDRVMAIYAPQPTWVIKADIQQFFDNLSWGVLLSQLERLKVAP | priestleyi Ana] |
| | AQVRLIEQQLKAGLILQGQFYRPNKGVLQGGILSGALANLYLSEF | |
| | DRLCQEAEIPLVRYGDDCVAVCHSYLQANRFLAMMQGWLEDIYLT | |
| | LNPDKTRIVGPDEGFVFLGHMF | |
| 8635 | GVDGITVDLFKGIAQEQIRLLHQQMRQERYVASPAKGFYLPKKTG | >PF_WP_00831 |
| | GDRLIGIPTVKDRIVQRYLLQGIYPHLENTFSEATFAYRPGLSIY | 2855. |
| | TAVAQVMTRYRHQPAWVIKADIQQFFDRLSWPLLLHQLDQLPLPP | [Leptolyngbya |
| | VWMRWIEQQLKAGIVIRGHFQRPNQGVLQGSILSGALANLYLNDF | sp. PCC6406] |
| | DRRCLAADIDLVRYGDDCVAVCQSYLEATRSLALMQDWIEDLYLS | |
| | LHPEKTQIIPPGEAFVFLGHRF | |
| 8636 | GIDGIPTDLFAGVVDEELSLLQRQLQQEYYQADPAKGFYRQKKSG | >PF_WP_024971209.1 |
| | GNRLIGIPTVRDRIVQRLLLHSIYPALEDVFSDRSYAYRPGLGVQ | [Microcystis |
| | SAIAHLSEVYAGQTVWTIKADVSRFFDSLNWALLLTRLERLSLEP | aeruginosa] |
| | VIVRMIEQQIKSGIVIDGQKLRQTKGVLQGGILSGALANLYLSDF | |
| | DARCVGLNLDLVRYGDDFVIVTSGLLEATRVLDSLHHWLADIYLA | |
| | LQPEKTRIIAPDGEFTFLGYQF | |
| 8637 | GFYRVKKSGGHRLIGIPTVRDRIVQRLLLRSLYPILEETFQDCSF | >W_Arthrospira |
| | AYRPGVGVKHAIERVAEVYSSQTWTIKADISQFFDSLCRTLLLSQ | platensis |
| | LEELSVDQTVVRYIKGQLEAGIVVGGMPILSGRGVLQGGILSGAL | 479129286 |
| | ANLYLSEFDRRCLDAGAYLTRYGDDFVIVARSLLEATRFLNLIED | |
| | WLSDIYLTLQPEKTHIFAPGEEFVFLGYGF | |
| 8638 | GITTDLFAGVKKDELIRLQQELIEEIYQPYPARGFYLPKNNGDKR | >W_[Cyanothece |
| | LLGIPAVRDRVVQRWLLEDLYLPLEEVFTDCSYAYRPGRGIQMAV | sp. _ PCC_7822]_ |
| | KHLYYYYQIQPKWIIKSDIRSFFDSLNWSILLSILEHLKLDPIIQ | 1307592471 |
| | QLVEQQLKSGIVLKGRYFPRNQGVLQGAVLSGALANLYLSEFDRK | |
| | CLEKGINLVRYGDDFVAACQSLGEAERTLNLITQWLERIYLQLHP | |
| | KKTEIYAPDQEFTFLGYLF | |
| 8639 | GIDNITVDLFAGVARYQLQVLLWQLQQENYFPRPAKGFYLRKASG | >PF_WP_00735 |
| | GKRLIGIPTVRDRIVQRFLLDELYWPLEDVFLDCSYAYRPGRGIQ | 5619.1 |
| | MAVKHLYSYYQFGQAWVIKADIEKFFDNLCWPLLLTDLEKLQFEP | [Kamptonema |
| | TLRQLIEQHLASGIVVKGQHFHPNQGVLQGGILSGALANLYLNEF | sp.] |
| | DRLCLSHGFNLVRFGDDFAVACADSIQANRCLEQINSWLGSFYLK | |
| | LQPEKTRIFAPDEEFTFLGYLF | |
| 8640 | GITTDLFAGVAKEQLYSLQRQLQQEHYAAHPALGFYLRKTRGGKR | >W_[Microcoleus |
| | LIGIPVVLDRIVQRLLLEELYLPLEDTFLDCSYAYRPGRGIQMAV | sp. _ PCC_7113] |
| | QHLESYYQFQPTWVIKADIAQFFDNLCHALLFTHLEQLQLEPIVL | 428314604 |
| | QLIEQQLKAGIVIKGQRLFPQKGVLQGAVLSGALANLYLTEFDRQ | |
| | CLSHGLNLVRYGDDFVVVAPDWIQANRALEQITTGLAQLYLTLQP | |
| | EKTKIFAPDEEFTFLGYQF | |
| 8641 | GISIGFFESMATEQLRNLVSQLQYGTYTASPAKGFYVPKKNGGKR | >W_Calothrix |
| | LIGIPTVRDRIIQRLLLDELYFPLEDTFVDCSYAYRPGRNIQQAV | parietina |
| | QHLYRYYQYQPKWIIKADIVEFFDNICLALLLNALEKLRLEPNIL | 428297029 |
| | QLIEQQIKSGIIINGQYQNAGKGLLQGGTLSGALANLYLTDFDQK | |
| | CLNQGINLVRYGDDFVIACSNFAEANRVLDKITGWLGGVYLTLKA | |
| | EKTEIFSPDDEFTFLGYRF | |
| 8642 | GISVDLFESMATEQLQNIAYQLKEETYTANPAKGFYIPKKNGTKR | >W _[Nostoc |
| | LIGIHTVRDRIIQRLLLDELYFPLEDTFLDCSYAYRPGHSIQQAV | sp. |
| | QHLYGYYQYQPKWIIKADVADFFDNLSWALLLTYLEELSLEPSLL | PCC_7120] |
| | QLLEQQLKSGIIIAGQYRNFGKGVLQGGILSGALANLYLTSFDRK | 17228961 |
| | CLSQGINLVRYGDDFVIACNSWLEANRILDKITGWLGEVYLTLQP | |
| | EKTQIFTPNDEFTFLGYRF | |
| 8643 | GISVELFESMATEQLQNIANQLYDETYTASPAKGFYIPKKNGSKR | >W_Calothrix |
| | LIGIPTVRDRIIQRLLLDELYFPLEDTFLDCSYAYRPGHNIHQAV | sp. 427717966 |
| | QHLYGYYQYQPKWIIKTDIADFFDNLSWALLLTALDELSLEPIVL | |
| | CLLEQQLHSGIIIAGQYRNFGKGVLQGGILSGALANLYLTNFDRK | |
| | CLSQSINLVRYGDDFVIACNSWQEANRILDKITTWLGEVYLTLQP | |
| | EKTQIFTPNEEFTFLGYRF | |
| 8644 | GVDGISLDLFESVAAEQLRNIEYQLHHETYTASPAKGFYVPKKNG | >PF_WP_02963 |
| | DKRLIGIPTVRDRIVQRLLLEELYFPLEDTFLDCSYAYRPGRNIQ | 0506.1[[Scytonema |
| | QAVQHLYSYYQLQPKWVIKADIAEFFDNLCWALLLTALEDLQLES | hofmanni] |
| | IVLQLLEGQLKSGIVIAGKPVYPGKGVLQGGVLSGALANLYLTNF | UTEXB1581] |
| | DRKCLSHGINLVRYGDDFAIACTSFHEANRILDKITTWLGELYLQ | |
| | LQPEKTQIYAPDDEFIFLGYRF | |
| 8645 | GVDGIDVDLFASAVNDQLRILLRQLQQESYCASPAKGFYLAKSSG | >PF_WP_03333 |
| | GKRLVGIPTVRDRIVQRLLLEELYFPLEDTFLDCSYAYRPGRNIQ | 4699.1 [Scytonema |
| | QAVQHLYSYYHLRPKWIIKADIAEFFDSLSWALLLTALEKLPLEP | hofmannii] |
| | IVVQLLEGQLRSGIVINGKPIYPGKGVLQGGVLSGALANLYLNEF | |
| | DKKCLHQGINLVRYGDDFAIACSNWREATRTLDKVAAWLGELYLN | |
| | LQPEKTQIFAPDDEFTFLGYRF | |
| 8646 | GVDGMTVDLFAAGVNEQLRILLRQLQQESYRASPAKGFFVAKKSG | >PF_WP_04103 |
| | GKRLIGIPTVRDRIVQRLLLEELYFPLEDTFLDCSYAYRPGRNIQ | 9832.1 |
| | QAVQHLYSYYQYQPKWIIKADIAEFFDNLCWALLFTALEDLQLEP | [Tolypothrix |
| | ILLQLLEQQLKSGIVIAGKPIYPGKGVLQGGVLSGALANLYLTSF | campylonemoides] |
| | ERKCLSYGINLVRYGDDFAIACSSWLEANRILDKITTWLGELYLN | |
| | LQPEKTQIFAPDDEFTFLGYRF | |
| 8647 | GISVDLFAASVDEQLTILLRQLQQESYHPSPAKGFYLTKKTGGKR | >W_Anabaena |
| | LVGIPTVQDRIVQRLLLEELYFPLEETFVDCSYAYRPGRNIQQAV | cylindric |
| | QQLFSYYQYHPTWIIKADIAQFFDNLCWALLLTNLEALQLESRIL | 440685177 |
| | QLLEQQLKAGIIIAGKHINFGKGVLQGGIISGALANLYLTIFDRK | |
| | CLSNGINLVRYGDDFAVACSSWKEANRILDKIIAWLGELYLTLQP | |
| | EKTQIFAPNEELKFLGYRF | |
| 8648 | GVDGITVDLFAASADQQLRIILRQLQQKSYRASPAKGFYLTKKSG | >PF_WP_04444 |
| | GKRLIGISTVRDRIVQRLLLEELYLPLEDTFVDCSYAYRPGCNIQ | 8019.1 |
| | QAVQRLFSYYQYHPTWIIKADIAQFFDNLSWALLFTGLETLHLEA | [Mastigocladus |
| | IVLELLEQQIKSGIVLGGKYINFGKGVLQGGILSGALANLYLTAF | laminosus] |
| | DRKCLSHGINLVRYGDDFAVACSSWTEANRILDKITTWLGGLYLT | |
| | LQPEKTQVFAPHEEFTFLGYRF | |
| 8649 | GVDCQSIASFESELQLGLNSILYDLRQQHYTPAALKRSQLKLPGK | >PF_WP_04600 |
| | KPRWLAFPTVRDRIVHTAIAILLQPYFEEEFEHNSYGYRPGRSYI | 7427.1 |
| | MAVDKVIEHRNQRRRHVFDADIQGYFDHIPQDKLLTKLQATAIDP | [Pseudoalteromonas |
| | TLIELIFTLLFSFQQSNDGLVFGKALGQGIPQGSAICPLLANFYL | rubra] |
| | DELDEHLNALGYHMVRYADDFVVCCDSAKAAQHAQYHTEQVLTHL | |
| | ALTLNLNKTQLTTFADGFKFLGHYF | |
| 8650 | GADGISIKEFASDLDTQLRQLHYDWKNNRYKPYRYRNITIEKANK | >PF_WP_03888 |
| | KPRELAVPTVRDRILHSALAQKLLNIFEAEFEHISYGYRPNRSYT | 4984.1 |
| | HAIRHIEQLRDQGYSTVIDADIQGYFDNICHIKLTELLNRHLPSD | [Vibrio |
| | WVSAITDTLLSQQQADGHLYFGAEIGVGIPQGSPLSPLLANLYLD | rotiferianus] |
| | GFDEALLDRGEQIIRYADDFVILLPNEDRAQSCLAFVTDYLNQLK | |
| | LTLNCEKTKVVSFQDGFTFLGVTF | |
| 8651 | GVTIQTFAIHLDTNLNTLLSAWNHGNYAPSPYRPLTIQPNEKKTR | >W_[Vibrio |
| | QLAIPTVADRIIHTAIAQKLVAKFEPEFEHISYGYRPNRSYTHAI | vulnificus] |
| | RHIEQLRNQGYLYVLDADIKGYFDHICHKRLKQILQKYLEDNWVE | 37677 |
| | SIMTLLLSQQMPAQTLLFGVELGRGIPQGSPLSPLLANLYLDGFD | 204 |
| | EALLDRGEQIVRYADDFVVLVTHEQQAQHCLAFVTQYLASLKLQL | |
| | NTEKTRVVSFQDGFTFLGVSF | |
| 8652 | GPDAVTILDFEAAWVDHMQQLAMELQSQIYRPLPPRRLFLDKRDG | >DS_gi\|1139391 |
| | GKRSIAILAVRDRIAQRAVLQILEPEIEPTFLDCSYGFRPYVGVP | 99\|ref\|ZP_01425057.1 |
| | HALTRIERYRQQGLQWVAHADISDCFGTIDHQILLSQLHQRISDR | [Herpetosiphon |
| | AVVELIGQWLSVGVMEDAATTEASNWWDDGEDLLERLAKHGEDLL | aurantiacus |
| | WPNQYPQAGPSYAPQMLDFEANRTDSLRKRALQGLASNAALWGIT | ATCC23779] |
| | HSKRVISGLRSLAPLFKQVPGGSLTWGAAGIATLALIPLSQRLLR | |
| | QHERGTLQGGAISPMLANIYLDSFDRAMTERGHILVRFADDFVLL | |
| | GAHQAAVEQALADATNVLKRLRLATKESKTGVQHFNDGLTFLGHR | |
| | F | |
| 8653 | GADEQTLAEFAADAEAQLGLLALQLTQGSYRPAPARLIPVAKPGG | >PF_KFB76584.1 |
| | GVRELLLPAVRDRIVQSALARYLADLLEPDFGEASHAYRPGHSVA | [Candidatus |
| | TALHRLQALRDGGLVFVAVCDIHHFFDSVDHRRLFSLLDDLPLER | Accumulibacter |
| | RLREQMKTCVRIEVADVQGQGAWSLARGLAQGSPLSPVLANLFLM | sp. SK02] |
| | AFDAACARAGLALVRYADDCVLACASETEAQSALAFAADALENIG | |
| | LALNTRKSRLASFAEGFEFLGAFC | |
| 8654 | GIDQITLHDFAADWPNQMVRLAEELRDGSYRPLPPRRVAIAKASG | >DS_gi\|7625862 |
| | GERAIAILTIRDRIAQRAVQQVLTPLFEPLFLDCSYGSRLAVGVP | 9\|ref\|ZP_007662 |
| | EAIERVVRYTEQGLIWVIDGDIRAYFDSIDHGILLGLLRQRIDEP | 83.1 |
| | AILHLIAQWLAVGSVHTETPDETLPDSPLVALLRRSGELIHEALN | |
| | APSDPLPTAYDYPDLSRPASPHSGIPTGLFAALSLAQPAFEIARQ | |
| | LTPLLKRIGAQRLAVGGALAVGTVLLSELVHRAQASHDRRGTLQG | |
| | GPLSPLLANIYLHPFDLAMTAHGARMVRFVDDFVVMCPDRTTAEH | |
| | TLVLVERQLATLRLTLNPQKTRIVAYAGGIEFLGQAL | |
| 8655 | GLDAVTLRDFEVDWTRQMAQLADELQQGTYRPLPAKRVAIPKASG | >DS_gi\|1486571 |
| | GERAIAILAVRDRVAQRAVQQVLDPLFDPCFLDCSYGCRPYVGVP | 22\|ref\|WP_01225 |
| | DAIARVQRYADQGLGWVVDADIATCFDSLDQRVLLSLVRQRIDEL | 9222.1\| |
| | PVLKLIAQWLEAGVLQGEAALPGDTPPTPLQRGEAAVRRALSWGA | [Chloroflexus |
| | ERLHPPPPVGPYAAAMWETPGGSIGEDGWAPRQPGLESHLWTAVM | aurantiacus] |
| | LARPVIDGARQALPYLQRIGGRRLAVAGAVAVGALALSEAAARLR | |
| | HASRRGVPQGGALSPLLANIYLHPFDVAMMGQGLRLVRFMDDFVV | |
| | MCATQEEAECALQFAQRQLHILRLTLNAEKTHITAYADGIEFLGA | |
| | AL | |
| 8656 | GADGVTIERYEGNLDLNLRIMRKELTEQTYFPLPLLRILVDKGNG | >DS_gi\|9120151 |
| | EARALCIPSVRDRIVQAAVLQLIEPVLEKEFEECSFAYRKGRSVK | 8\|emb\|CAJ74578.1 |
| | QAVYKVREYYEQGYQWVVDADIDAFFDSVDYSLLLLKFKCYIHDP | [Candidatus |
| | CIQNLVGLWLKGEVWDGKTVTTLKKGIPQGSPISPILANLYLDEF | Kuenenia |
| | DEELTRNGYKLVRFSDDFIILCKNSGMAKESLKLTKKILEKLLLE | stuttgartiensis] |
| | LDEEQVINFDQGFKFLGVIF | |
| 8657 | GVDYQTLAAFADRLHKNLETLRDEVNYETYQPQPLLRIELEKPGG | >PF_WP_02715 |
| | GTRPLSIPTVRDRILQTAVTRVIEPLFEAEFEDCSFAYRKGRSVD | 0711.1 |
| | QALDRIQLLQRQGYHWVVDADIQCFFDSIDHTLLMTMVGKLVTDV | [Methylobacter |
| | GLLRLIEQWLCATVVDGDRRFVLSKGVAQGSPIGPLLSNLYLHHL | tundripaludum] |
| | DEALLDNNLCLIRFADDFLILCKSQDHAEQALELTDSLLGELRLT | |
| | LNTRKTQIVHFNQGFRFLGVQF | |
| 8658 | GYDKQSITDYSWRIEEHLADLGRQLLTNTYEPQPLLKLVMLKPTG | >DS_gi\|6854873 |
| | KLRTLLIPTVMERVAQTAAAIVLTPLVESELGANTFAYRPGLSRM | 3\|ref\|ZP_005882 |
| | TAAREIERLRNLGYNWVVDADISSFFDTVDHPLLFQRFRELCDDE | 02.1 |
| | ELLTLIARWLTAEIVDGQNPKVKNTIGLPQGCPISPMLANLYLDK | [Pelodictyon |
| | FDERMEQEGFKLVRFADDFLILCKSKPKAEAALQLSESALAELKL | phaeoclathratiforme |
| | QLNNEKTRITTFAEGFKYLGYLF | BU1] |
| 8659 | GWDNTSIQDYSLRLEENLKSLSHALLTGTYRQSPLLKLVMLKPDG | >DS_gi\|1193578 |
| | KERVLLIPGVIDRVAQTAASIVLSPIIEAELGNCTFAYRPGISRE | 46\|ref YP_91249 |
| | GAAREIDRLHREGYQWVLDADIRNFFDNVRHDLLFQRLVELVDDK | 0.1 |
| | EMISLLHRWLTAEIVDGLNPRTRNTMGLPQGCPISPALANLYLDR | [Chlorobiumphaeo |
| | FDETMEQQGFKLVRFADDYLVLCKTRPKAEAALKLSESALAELKL | bacteroides |
| | ELHSDKTRITTFAEGFKYLGYLF | DSM266] |
| 8660 | GCDGEEVEQFAQGLLGRLHTLQAEVADGRYVARPLRVVALPKPSG | >PF_WP_00985 |
| | GQRLLAIPGVRDRVLQAAMAHALGRRIEPTLDEASHAYRPGRSVL | 5610.1 |
| | GALAALLALRDQGRSTVLKADVASFFDRIHQPTLLAQLRRFSADP | [Rubrivivax |
| | GLLALVGQVLAAVLDDDGERRLMTRGVPQGSPLSPLLANLYLHPF | benzoatilyticus] |
| | DVGMRAQGFQLIRYADDLVLACLDADEAARAQDAAARALRELHLE | |
| | LNPAKTRIASFVSGFDFLGVRF | |
| 8661 | GGDGEGVATFQAGLDLRLARLAADLLGGTYRPGPWLIAGGAVVAP | >PF_WP_06276 |
| | VADRVVMTAVATGLPDPSSDGDPAAVMARLAALGQQGAVHLLDGT | 3150.1 |
| | ITHVTDLVPHDLLCERLAALGGDARLVDLFGMWLAVADPEDGLGI | [Tistrella |
| | PPGLPVSGLLARLHLGAVAARIAAAGVHLVPAAGEILVLATGAAA | mobilis] |
| | AEDARGRMLALLADHGLYVDVDLPRMIRLDQAGPRLGRIM | |
| 8662 | GVDRESVVHFAKNSEAYLSQLRRSLASGYYHPMPLRQLFIPKKAG | >PF_WP_00962 |
| | GWRELGVPTVRDRIVQHALLNILHPLLEPQFEACSFAYRPGRSHL | 5648.1 |
| | SAVRQIAQWRDRGYEWVLDADVVRYFENILWQRLLDEVAERLAAP | [Pseudanabaena |
| | EVLSLISAWLSVGVLSKEGLMFPQKGISQGSAISPILANVYLDDF | biceps] |
| | DEIVTATGLKLVRYADDFVVMSRSQKRIVEAKDEVADLMNGIGLQ | |
| | LHPDKTRIVDFDRGFRFLGHAF | |
| 8663 | RVPTVRDRIVQQALLNVLHPVLEPQFEPVSFAYRPGRSHKLAVEK | >PF_WP_00651 |
| | VSAWHRRGYDWLLDGDIVSYFDQVEHSRLLSEVDERLGASDFETL | 5493.1 [Leptolyngbya |
| | ALRLIEQWNTVGTLTSAGLVLPERGIPQGSVVSPILANVYLDDFD | sp. |
| | EALQASRFKLVRFADDFVVMGRSQRQAEQAQAKVAELLTTMGLQL | PCC7375] |
| | HPDKTQITNFDRGFRFLGHAF | |
| 8664 | GVDGETIYAFGLHKSRNLTRLLQQVATSTYRPLPLRQFFIPKKSG | >PF_WP_01730 |
| | GWRELGVPTVRDRIVQQALLQVLHPVFEVEFEPQSYAYRPGRSHR | 2244.1 [Nodosilinea |
| | MAVERVAHWRSRGYDWVLDADIVKYFDTLQHPRLLAEVKERLNQP | nodulosa] |
| | WVLALLQGWITAGTLTREGILLPTCGVPQGSPISPLLANVYLDDF | |
| | DELLTQAGHKLVRYADDFVVLARTQQRLVEAQTYVAQLLEGMGLS | |
| | LHPNKTQITTFDRGFRFLGHAF | |
| 8665 | RIPAVADRIVQQALLNVLYPILEPEFEVCSFAYRPGRSHRMAVDQ | >PF_WP_04544 |
| | IHAFSRRGYRWVMEADIFDYFDHIGHRRLLAEVAERLPGQDPSFC | 2561.1[Synechococcus |
| | DLVLQLVQQWIAVGVVTQSGLILPQAGIPQGAVIAPILANVYLDD | sp. |
| | FDEALLRTPLKLVRYADDFVILGQRERQVQKILPEVAQQMAEIGL | NKBG042902] |
| | QLNMSKTRITNFQKGFKFLGHIF | |
| 8666 | AIGHLVEQWIGSGVSTASGLILPNKGVPQGAVISPILANVYFDDF | >PF_BAU44853.1 |
| | DEAIEAAGLKLVRYADDFVILAKSKARIERAYNLVASLLHAMGLE | [Leptolyngbya |
| | LHPDKTRVTTFNEGFRFLGHTF | sp. 077] |
| 8667 | AVDRISALRRMGYTWVVEADIEKAFDRIPHDPVLEALDTALDPAP | >W_[Rhodobacter |
| | GTRALIDLVGLWLAHGSGQLGTPGRGLAQGSPLSPLLSNLFFDGL | capsulatus]29467 |
| | DDRFDSGAARIVRFADDFVILARSEAGAEEARALAEEFVAGHGLR | 6823 |
| | MVSRETRVVGFDRGFQFLGQLF | |
| 8668 | GGDGQTLAQFQRTVLLHLHRLGDDVRAGLYMPGPHRVVSIPKRAG | >PF_WP_01996 |
| | GWRSLSIPCVRDRVLQTAVAQRLQPILEPEFEPESYGYRPGRSVA | 0649.1 [Woodsholea |
| | QAIARVATLRRQGFRWTVDADIERFFDCVPHGPLLERLRPFLGDP | maritima] |
| | GLVGLVEMWLAGAGPHGRGLPQGSPISPLLANLYLDDVDEGLKST | |
| | HTRLVRFADDFVILTRNEDEALQALERARGLLDKLGLSLNLEKTR | |
| | IVPFEGGLDFLGRKF | |
| 8669 | GGDGVTIDAFDAIAEPRLQALHAALASGGYWPAPARVIEAKKPSG | >PF_WP_01995 |
| | GTRTLRIPAIVDRVVQTAAALVLTPILDREFEDASFGYRPGRSVG | 6891.1[Loktanella |
| | QAVARVAYLRNAGYVWTVDGDIRAFFDEVPHAPLLDRVDRVLGCA | vestfoldensis] |
| | RTADLVERWLQVYCDGGRGLPQGMPLSPVLSNLYLDSIDEKIEKG | |
| | GVRLVRFADDFLLLCRSEAVAEGALARMTGILREAGLKIHPEKTA | |
| | IRRFEDATRFLGHMF | |
| 8670 | GESLDAFHIGVEPRLARLAADVRGGTYRPGPYRLLDVPKDDGGTR | >W_[Tistrella |
| | RLAIPCVADRVLMTSAALVMGPMLDATFEPSSHGYRPGRGVRTAI | mobilis] |
| | ARVESLRDQGFHWVLDADITRFFDRVPHDRLLDRLQQATGDARLV | 389875622 |
| | DLVGLWLDGYDREGEAGRGDGLGLPQGSPVSPLLANLYLDTVDER | |
| | IAAAGLHLVRFADDFVILAADEAAAEGARAHVAALLADHGLHLHP | |
| | DKTRVVSFDQGFAFLGKLF | |
| 8671 | RQTLDDFAESLERNLEGLHAALRSASYRPGPIRNVSIPKRDGSPR | >W_[Desulfarculus |
| | RLSIPSVADRVVQTALCQGLTPILEPEMEDASFAYRPGRSVQMAV | baarsii] |
| | ERVGRYFRQGYHWVVDGDIDDYFDSIPHHGLMAVLRRYVDDQDVL | 302343124 |
| | GLIAQWLAHAHAGGVGVSQGSPLSPLLANIYLDDMDERIGRTGAR | |
| | LVRFADDFLLLCKSEERARESLAAMSALLAEYGLGLNPDKTRIVN | |
| | FEQGFEFLGRLF | |
| 8672 | GGDGETIAHFARQAEFRLARLAHELQADLYRPGPLRQISVPKRKG | >PF_KQB14189.1 |
| | EGMRVLSIPCVVDRIAQRATAAVLSAALEPQFSDASFGYRPGRSV | [Rhodobacter |
| | AQAVARVDALRRQGFTWVVDADIKAFFDSVPHAPLAARLHAAGIE | capsulatus] |
| | PQLIELIDLWLDSFSAEGVGLAQGSPISPVLANLHLDALDDSFGP | |
| | RGSVRIVRFADDFVLLTRCRPGAEAALAKARDQLAEAGLRLNLAK | |
| | TRIVPYDQALRFLGHLF | |
| 8673 | GGDGMTVARFALVAESMIQRLAGALRSGQYRPGPARRAFIPKKDG | >PF_EJW09481.1_(2) |
| | GLRPLDIPCVHDRVVQGAATLVLDPVLDKAFADSSFAYRRGRSVA | [Rhodovulum |
| | QAVARIGSLRRQGFTHVVDGDIRAYFERIPHDRLITKLEQHVDDQ | sp. PH10] |
| | AMVDLIWLWLETYSLTGRGVPQGAPISPLLANLYLDAVDDRIERA | |
| | GVRLVRFADDFVLLAKTPASAEKALVEMTRLLAEEGLEIHPEKTR | |
| | LVSFEEGFRFLGHVF | |
| 8674 | GGDGVPLARFLVNAPARIARLSAGLRDGSYAPGPLRRVDIPKKSG | >PF_EJW09347. |
| | GTRPLAIPCVVDRIAQTAVMQALAPRLDEEFAESSFGYRLGRGVR | 1_(2) |
| | DAVKRVAALRGKGHVYVVDADIAKFFESVPHDKLLERLAQSMTDG | [Rhodovulum |
| | PLMRLIGLWIEHGGARGRGLPQGSPLSPLLANLYLDRLDDAFAKR | sp. |
| | GAHIVRFADDFVILAESRHGAEGALVRAEKLLAEHGLSLNREKTR | PH10] |
| | VTSFDQGFRFLGHLF | |
| 8675 | GGDGVTIDRFARRAPQRLTALSGALLDGRYRPGDLRRIDLKKRDG | >PF_WP_06083 |
| | GTRPLAIPSVIDRVAQTAAALVLTPILDPLFDEASFGYRPGRSVA | 6241.1 |
| | MAVRRIDMLRRRGFCHVVEADIVRCFERIPHEPVLSSLAKTLAGR | [Rhodovulum |
| | VGADRLVDLVALWLEHAAMFLETPGLGLAQGSPLSPLLSNLYLDR | sulfidophilum] |
| | LDDALDRRDVAVVRFADDFVLLCRSREAAAKALNRAENLLEAHGL | |
| | ELHGDGTRIVDFDRGFEFLGHLF | |
| 8676 | GLTVGRFAEAAPSRLLALHRTLRMGDYRPGPLRRLSIPKPDGALR | >W_Azospirillum |
| | PLAIPPVTDRVAQTAAALVLTPLLDGEFEDASFGYRPGRSVPQAV | lipoferum |
| | ARVARWRDQGYDWVVDADIERYFERVPHDRLLIRLERSIGAGPLT | 1374998939 |
| | ELIAVWLESGAENGVGLPQGSPLSPLLSNLYLDDLDEALDGRGLR | |
| | LVRFADDFVLLCRSRERAERALDHAAAVLEEHGLRLNRDKTRIVP | |
| | FDQGFRFLGHLF | |
| 8677 | GMTVEEFSIDLPTRLVRLQLALAQGTYRPGRLRRVDVAKEDGGTR | >W_Azospirillum |
| | PLAIPPVVDRVAQTAVAQVLTPLLDPRMHDGSFAYRPGRSVAMAV | lipoferum_ |
| | ARVAEHRRQGFGWVVDGDIERYFERVPHERMMACLARVIDEPPLL | 2288957883 |
| | DLIELWLESFSAMGLGLPQGAPLSPLLANLYLDDIDDRIAARGVR | |
| | LVRFADDFLLMCRGEAAAEDARDRMAALLAEHGLRLHPDKTRIVP | |
| | FEQGFRFLGHLF | |

### Programmable DNA Binding Domain

In some embodiments, the DNA-binding domain of a split prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a split prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprise a nuclease activity. In some embodiments, the DNA-binding domain of a split prime editor comprises an endonuclease domain having single strand DNA cleavage activity. For example, the endonuclease domain may comprise a FokI nuclease domain. In some embodiments, the DNA-binding domain of a split prime editor comprises a nuclease having full nuclease activity. In some embodiments, the DNA-binding domain of a split prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a split prime editor has nickase activity. In some embodiments, the DNA-binding domain of a split prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. In some embodiments, the Cas protein has nickase activity. A Cas protein may be a Class 1 or a Class 2 Cas protein. A Cas protein can be a type I, type II, type III, type IV, type V Cas protein, or a type VI Cas protein. Non-limiting examples of Cas proteins include Cas9, Cas 12a (Cpf1), Cas12e (CasX), Cas12d (CasY), Cas12b1 (C2c1), Cas12b2, Cas12c (C2c3), C2c4, C2c8, C2c5, C2c10, C2c9, Cas14a, Cas14b, Cas14c, Cas14d, Cas14c, Cas14f, Cas 14g, Cas 14h, Cas 14u, Cns2, Cas @, and homologs, functional fragments, or modified versions thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

A Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. A Cas protein, e.g., Cas9, can comprise an amino acid change such as a deletion, insertion, substitution, fusion, chimera, or any combination thereof relative to a wild-type version of the Cas protein. A Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a split prime editor comprises a Cas protein, e.g., Cas9, wherein all nuclease domains of the Cas protein are active. In some embodiments, a split prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PERNA.

In some embodiments, a split prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a split prime editor comprises a Cas nickase comprising two nuclease domains (e.g., Cas9), with one of the two nuclease domains modified to lack catalytic activity or deleted. In some embodiments, the Cas nickase of a split prime editor comprises a nuclease inactive RuvC domain and a nuclease active HNH domain. In some embodiments, the Cas nickase of a split prime editor comprises a nuclease inactive HNH domain and a nuclease active RuvC domain. In some embodiments, a split prime editor comprises a Cas9 nickase having an amino acid substitution in the RuvC domain. In some embodiments, the Cas9 nickase comprises a D10X amino acid substitution compared to a wild type S. pyogenes Cas9, wherein X is any amino acid other than D. In some embodiments, a split prime editor comprises a Cas9 nickase having an amino acid substitution in the HNH domain. In some embodiments, the Cas9 nickase comprises a H840X amino acid substitution compared to a wild type S. pyogenes Cas9, wherein X is any amino acid other than H.

In some embodiments, a split prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a split prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a split prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a split prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a split prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide. In some embodiments, a Cas9 polypeptide is a SaCas9 polypeptide. In some embodiments, a Cas9 polypeptide is a ScCas9 polypeptide. In some embodiments, a Cas9 polypeptide is a StCas9 polypeptide. In some embodiments, a Cas9 polypeptide is a SluCas9 polypeptide. In some embodiments, a Cas9 polypeptide is a NmCas9 polypeptide. In some embodiments, a Cas9 polypeptide is a CjCas9 polypeptide. In some embodiments, a Cas9 polypeptide is a FnCas9 polypeptide. In some embodiments, a Cas9 polypeptide is a TdCas9 polypeptide. In some embodiments, a Cas9 polypeptide is a chimera comprising domains from two or more of the organisms described herein or those known in the art. In some embodiments, a Cas9 polypeptide is a Cas9 polypeptide from Streptococcus macacae. In some embodiments, a Cas9 polypeptide is a Cas9 polypeptide generated by replacing a PAM interaction domain of a SpCas9 with that of a Streptococcus macacae Cas9 (Spy-mac Cas9).

An exemplary Streptococcus pyogenes Cas9 (SpCas9) amino acid sequence is provided in SEQ ID NO: 4449.

Exemplary Streptococcus pyogenes Cas9 (SpCas9) amino acid sequence:

SEQ ID NO: 4449
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD.

In some embodiments, a split prime editor comprises a Cas9 protein from Staphylococcus lugdunensis (Slu Cas9). An exemplary amino acid sequence of a Slu Cas9 is provided in SEQ ID NO: 4450.

Exemplary Staphylococcus lugdunensis Cas9 (Slu Cas9) amino acid sequence WP_002460848.1:

(SEQ ID NO: 4450)
MKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEEASKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKK.

In some embodiments, a Cas9 protein comprises a variant Cas9 protein containing one or more amino acid substitutions. In some embodiments, a wildtype Cas9 protein comprises a RuvC domain and an HNH domain. In some embodiments, a split prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a split prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a split prime editor comprises a Cas9 that has a non-functional HNH domain and a functional RuvC domain. In some embodiments, the split prime editor can cleave the edit strand (i.e., the PAM strand), but not the non-edit strand of a double stranded target DNA sequence. In some embodiments, a split prime editor comprises a Cas9 having a non-functional RuvC domain that can cleave the target strand (i.e., the non-PAM strand), but not the edit strand of a double stranded target DNA sequence. In some embodiments, a split prime editor comprises a Cas9 that has neither a functional RuvC domain nor a functional HNH domain, which may not cleave any strand of a double stranded target DNA sequence.

In some embodiments, a split prime editor comprises a Cas9 having a mutation in the RuvC domain that reduces or abolishes the nuclease activity of the RuvC domain. In some embodiments, the Cas9 comprise a mutation at amino acid D10 as compared to a wild type SpCas9 as set forth in SEQ ID NO: 4449, or a corresponding mutation thereof. In some embodiments, the Cas9 comprise a D10A mutation as compared to a wild type SpCas9 as set forth in SEQ ID NO: 4449, or a corresponding mutation thereof. In some embodiments, the Cas9 polypeptide comprise a mutation at amino acid D10, G12, and/or G17 as compared to a wild type SpCas9 as set forth in SEQ ID NO: 4449, or a corresponding mutation thereof. In some embodiments, the Cas9 polypeptide comprise a D10A mutation, a G12A mutation, and/or a G17A mutation as compared to a wild type SpCas9 as set forth in SEQ ID NO: 4449, or a corresponding mutation thereof.

In some embodiments, a split prime editor comprises a Cas9 polypeptide having a mutation in the HNH domain that reduces or abolishes the nuclease activity of the HNH domain. In some embodiments, the Cas9 polypeptide comprise a mutation at amino acid H840 as compared to a wild type SpCas9 as set forth in SEQ ID NO: 4449, or a corresponding mutation thereof. In some embodiments, the Cas9 polypeptide comprise a H840A mutation as compared to a wild type SpCas9 as set forth in SEQ ID NO: 4449, or a corresponding mutation thereof. In some embodiments, the Cas9 polypeptide comprise a mutation at amino acid E762, D839, H840, N854, N856, N863, H982, H983, A984, D986, and/or a A987 as compared to a wild type SpCas9 as set forth in SEQ ID NO: 4449, or a corresponding mutation thereof. In some embodiments, the Cas9 polypeptide comprise a E762A, D839A, H840A, N854A, N856A, N863A, H982A, H983A, A984A, and/or a D986A mutation as compared to a wild type SpCas9 as set forth in SEQ ID NO: 4449, or a corresponding mutation thereof.

In some embodiments, a split prime editor comprises a Cas9 having one or more amino acid substitutions in both the HNH domain and the RuvC domain that reduce or abolish the nuclease activity of both the HNH domain and the RuvC domain. In some embodiments, the split prime editor comprises a nuclease inactive Cas9, or a nuclease dead Cas9 (dCas9). In some embodiments, the dCas9 comprises a H840X substitution and a D10X mutation compared to a wild type SpCas9 as set forth in SEQ ID NO: 4449 or corresponding mutations thereof, wherein X is any amino acid other than H for the H840X substitution and any amino acid other than D for the D10X substitution. In some embodiments, the dead Cas9 comprises a H840A and a D10A mutation as compared to a wild type SpCas9 as set forth in SEQ ID NO: 4449, or corresponding mutations thereof.

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a split prime editor comprises a Cas protein, e.g., Cas9, containing modifications that allow altered PAM recognition. In prime editing using a Cas-protein-based split prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the split prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a split prime editor recognizes a canonical PAM, for example, a SpCas9 recognizes 5′-NGG-3′ PAM. In some embodiments, the Cas protein of a split prime editor has altered or non-canonical PAM specificities. Exemplary PAM sequences and corresponding Cas variants are described in Table 1 below. It should be appreciated that for each of the variants provided, the Cas protein comprises one or more of the amino acid substitutions as indicated compared to a wild type Cas protein sequence, for example, the Cas9 as set forth in SEQ ID NO: 4449. The PAM motifs as shown in Table 1 below are in the order of 5′ to 3′.

TABLE 1
Cas protein variants and corresponding PAM sequences

| Variant | PAM |
|---|---|
| spCas9 (wild type) | NGG, NGA, NAG, |
| | NGNGA |
| spCas9- VRVRFRR R1335V, L1111R, D1135V, | NG |
| G1218R, E1219F, A1322R, T1337R | |
| spCas9-VQR (D1135V, R1335Q, T1337R) | NGA |
| spCas9-EQR (D1135E, R1335Q, T1337R) | NGA |
| spCas9-VRER (D1135V, G1218R, R1335E, T1337R) | NGCG |
| spCas9-VRQR (D1135V, G1218R, R1335Q, T1337R) | NGA |
| Cas9-NG (L1111R, D1135V, G1218R, E1219F, | NGN |
| A1322R, T1337R, R1335V) | |
| SpG Cas9 (D1135L, S1136W, G1218K, E1219Q, | NGN |
| R1335Q, T1337R) | |
| SyRY Cas9 | NRN |
| (A61R, L1111R, N1317R, A1322R, and R1333P) | |
| xCas9 (E480K, E543D, E1219V, K294R, Q1256K, | NGN |
| A262T, S409I, M694I) | |
| SluCa9 | NNGG |
| sRGN1, sRGN2, sRGN4, sRGN3.1, sRGN3.3 | NNGG |
| saCas9 | NNGRRT, NNGRRN |
| saCas9-KKH (E782K, N968K, R1015H) | NNNRRT |
| spCas9-MQKSER (D1135M, S1136Q, G1218K, E1219S, | NGCG/NGCN |
| R1335E, T1337R) | |
| spCas9-LRKIQK (D1135L, S1136R, G1218K, E1219I, | NGTN |
| R1335Q, T1337K) | |
| spCas9-LRVSQK (D1135L, S1136R, G1218V, E1219S, | NGTN |
| R1335Q, T1337K) | |
| spCas9-LRVSQL(D1135L, S1136R, G1218V, E1219S, | NGTN |
| R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a split prime editor comprises a Cas9 polypeptide comprising one or mutations selected from the group consisting of: A61R, L111R, D1135V, R221K, A262T, R324L, N394K, S4091, S4091, E427G, E480K, M495V, N497A, Y515N, K526E, F539S, E543D, R654L, R661A, R661L, R691A, N692A, M694A, M694I, Q695A, H698A, R753G, M763I, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, 11322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 4449.

In some embodiments, a split prime editor comprises a SaCas9 polypeptide. In some embodiments, the SaCas9 polypeptide comprises one or more of mutations E782K, N968K, and R1015H as compared to a wild type SaCas9. In some embodiments, a split prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a split prime editor comprises a Sc Cas9, for example, a wild type ScCas9 or a ScCas9 polypeptide comprises one or more of mutations I367K, G368D, I369K, H371L, T375S, T376G, and T1227K as compared to the wild type ScCas9. In some embodiments, a split prime editor comprises a St1 Cas9 polypeptide, a St3 Cas9 polypeptide, or a S1u Cas9 polypeptide.

In some embodiments, a split prime editor comprises a Cas polypeptide that comprises a circular permutant Cas variant. For example, a Cas9 polypeptide of a split prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures:
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus, or the corresponding circular permutants of other Cas9 proteins (including other Cas9 orthologs, variants, etc.).

In some embodiments, a circular permutant Cas9 comprises any one of the following structures (amino acid positions as set forth in SEQ ID NO: 4449-1368 amino acids of UniProtKB—Q99ZW2:
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 4449-1368 amino acids of UniProtKB—Q99ZW2 N-terminus-[103-1368]-[optional linker]-[1-102]-C-terminus:
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the 95% or more of the C-terminal amino acids of a Cas9 (e.g., amino acids about 1300-1368 as set forth in SEQ ID No: 4449 or corresponding amino acid positions thereof), or the 90%, 85%, 80%, 75%, 70%, 65%, 60%, 55%, 50%, 45%, 40%, 35%, 30%, 25%, 20%, 15%, 10%, or 5% or more of the C-terminal amino acids of a Cas9 (e.g., SEQ ID No: 4449). The N-terminal portion may correspond to 95% or more of the N-terminal amino acids of a Cas9 (e.g., amino acids about 1-1300 as set forth in SEQ ID No: 4449 or corresponding amino acid positions thereof), or 90%, 85%, 80%, 75%, 70%, 65%, 60%, 55%, 50%, 45%, 40%, 35%, 30%, 25%, 20%, 15%, 10%, or 5% or more of the N terminal amino acids of a Cas9 (e.g., as set forth in SEQ ID No: 4449 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID No: 4449 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., as set forth in SEQ ID No: 4449 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID No: 4449 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (c/g/as set forth in SEQ ID No: 4449 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID No: 4449 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 4449: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID No: 4449 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 18, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a split prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a split prime editor comprises a SpCas9 that is 1368 amino acids in length and has a predicted molecular weight of 158 kilodaltons. In some embodiments, a split prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 18). In various other embodiments, the napDNAbp can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof.

TABLE 2
Exemplary Cas proteins

| | |
|---|---|
| Legacy nomenclature | Current nomenclature |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

In some embodiments, a split prime editor as described herein may comprise a Cas12a (Cpf1) polypeptide or functional variants thereof. In some embodiments, the Cas 12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas 12a polypeptide.

In some embodiments, a split prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas 12e, a Cas12d, a Cas13, Cas14a, Cas14b, Cas14c, Cas14d, Cas14e, Cas14f, Cas14g, Cas14h, Cas14u, or a Cas Φ polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas Φ protein. In some embodiments, the Cas protein is a Cas12e, Cas12d, Cas13, or Cas @ nickase.

In some embodiments, the Cas protein comprises any one of the Cas9 amino acid sequences as set forth in Table 14. In some embodiments, the Cas protein comprises a Cas12 amino acid sequence as set forth in Table 14.

In some embodiments, the DNA binding domain comprises any one of the sequences set forth in Table 14.

TABLE 14
Exemplary DNA-binding domain nuclease and nickase sequences;
for each DNA binding domain nuclease, sequences of an
active nuclease and a nickase are provided

| SEQ | | | SEQ | | | WT | |
|---|---|---|---|---|---|---|---|
| ID | Nuclease | Nickase | ID | Nickase | | Uniprot/ | |
| NO: | sequence | Mutation | NO: | Sequence | Name | NCBI | PAM |
| 8678 | DKKYSIGLDIGTNS | H840A | 8000 | DKKYSIGLDIGT | SpCas9 | Q99ZW2-1 | NGG |
| | VGWAVITDEYKVP | | | NSVGWAVITDE | | | |
| | SKKFKVLGNTDRH | | | YKVPSKKFKVL | | | |
| | SIKKNLIGALLFDSG | | | GNTDRHSIKKNL | | | |
| | ETAEATRLKRTARR | | | IGALLFDSGETA | | | |
| | RYTRRKNRICYLQE | | | EATRLKRTARR | | | |
| | IFSNEMAKVDDSFF | | | RYTRRKNRICYL | | | |
| | HRLEESFLVEEDKK | | | QEIFSNEMAKVD | | | |
| | HERHPIFGNIVDEV | | | DSFFHRLEESFL | | | |
| | AYHEKYPTIYHLRK | | | VEEDKKHERHPI | | | |
| | KLVDSTDKADLRLI | | | FGNIVDEVAYHE | | | |
| | YLALAHMIKFRGH | | | KYPTIYHLRKKL | | | |
| | FLIEGDLNPDNSDV | | | VDSTDKADLRLI | | | |
| | DKLFIQLVQTYNQL | | | YLALAHMIKFR | | | |
| | FEENPINASGVDAK | | | GHFLIEGDLNPD | | | |
| | AILSARLSKSRRLE | | | NSDVDKLFIQLV | | | |
| | NLIAQLPGEKKNGL | | | QTYNQLFEENPI | | | |
| | FGNLIALSLGLTPNF | | | NASGVDAKAILS | | | |
| | KSNFDLAEDAKLQ | | | ARLSKSRRLENL | | | |
| | LSKDTYDDDLDNL | | | IAQLPGEKKNGL | | | |
| | LAQIGDQYADLFLA | | | FGNLIALSLGLT | | | |
| | AKNLSDAILLSDILR | | | PNFKSNFDLAED | | | |
| | VNTEITKAPLSASM | | | AKLQLSKDTYD | | | |
| | IKRYDEHHQDLTLL | | | DDLDNLLAQIG | | | |
| | KALVRQQLPEKYK | | | DQYADLFLAAK | | | |
| | EIFFDQSKNGYAGY | | | NLSDAILLSDILR | | | |
| | IDGGASQEEFYKFI | | | VNTEITKAPLSA | | | |
| | KPILEKMDGTEELL | | | SMIKRYDEHHQ | | | |
| | VKLNREDLLRKQR | | | DLTLLKALVRQ | | | |
| | TFDNGSIPHQIHLGE | | | QLPEKYKEIFFD | | | |
| | LHAILRRQEDFYPF | | | QSKNGYAGYID | | | |
| | LKDNREKIEKILTFR | | | GGASQEEFYKFI | | | |
| | IPYYVGPLARGNSR | | | KPILEKMDGTEE | | | |
| | FAWMTRKSEETITP | | | LLVKLNREDLLR | | | |
| | WNFEEVVDKGASA | | | KQRTFDNGSIPH | | | |
| | QSFIERMTNFDKNL | | | QIHLGELHAILR | | | |
| | PNEKVLPKHSLLYE | | | RQEDFYPFLKDN | | | |
| | YFTVYNELTKVKY | | | REKIEKILTFRIP | | | |
| | VTEGMRKPAFLSG | | | YYVGPLARGNS | | | |
| | EQKKAIVDLLFKTN | | | RFAWMTRKSEE | | | |
| | RKVTVKQLKEDYF | | | TITPWNFEEVVD | | | |
| | KKIECFDSVEISGVE | | | KGASAQSFIERM | | | |
| | DRFNASLGTYHDL | | | TNFDKNLPNEK | | | |
| | LKIIKDKDFLDNEE | | | VLPKHSLLYEYF | | | |
| | NEDILEDIVLTLTLF | | | TVYNELTKVKY | | | |
| | EDREMIEERLKTYA | | | VTEGMRKPAFL | | | |
| | HLFDDKVMKQLKR | | | SGEQKKAIVDLL | | | |
| | RRYTGWGRLSRKLI | | | FKTNRKVTVKQ | | | |
| | NGIRDKQSGKTILD | | | LKEDYFKKIECF | | | |
| | FLKSDGFANRNFM | | | DSVEISGVEDRF | | | |
| | QLIHDDSLTFKEDI | | | NASLGTYHDLL | | | |
| | QKAQVSGQGDSLH | | | KIIKDKDFLDNE | | | |
| | EHIANLAGSPAIKK | | | ENEDILEDIVLTL | | | |
| | GILQTVKVVDELV | | | TLFEDREMIEER | | | |
| | KVMGRHKPENIVIE | | | LKTYAHLFDDK | | | |
| | MARENQTTQKGQK | | | VMKQLKRRRYT | | | |
| | NSRERMKRIEEGIK | | | GWGRLSRKLIN | | | |
| | ELGSQILKEHPVEN | | | GIRDKQSGKTIL | | | |
| | TQLQNEKLYLYYL | | | DFLKSDGFANR | | | |
| | QNGRDMYVDQEL | | | NFMQLIHDDSLT | | | |
| | DINRLSDYDVDHIV | | | FKEDIQKAQVSG | | | |
| | PQSFLKDDSIDNKV | | | QGDSLHEHIANL | | | |
| | LTRSDKNRGKSDN | | | AGSPAIKKGILQ | | | |
| | VPSEEVVKKMKNY | | | TVKVVDELVKV | | | |
| | WRQLLNAKLITQR | | | MGRHKPENIVIE | | | |
| | KFDNLTKAERGGL | | | MARENQTTQKG | | | |
| | SELDKAGFIKRQLV | | | QKNSRERMKRIE | | | |
| | ETRQITKHVAQILD | | | EGIKELGSQILKE | | | |
| | SRMNTKYDENDKL | | | HPVENTQLQNE | | | |
| | IREVKVITLKSKLVS | | | KLYLYYLQNGR | | | |
| | DFRKDFQFYKVREI | | | DMYVDQELDIN | | | |
| | NNYHHAHDAYLN | | | RLSDYDVDAIVP | | | |
| | AVVGTALIKKYPKL | | | QSFLKDDSIDNK | | | |
| | ESEFVYGDYKVYD | | | VLTRSDKNRGK | | | |
| | VRKMIAKSEQEIGK | | | SDNVPSEEVVK | | | |
| | ATAKYFFYSNIMNF | | | KMKNYWRQLL | | | |
| | FKTEITLANGEIRKR | | | NAKLITQRKFDN | | | |
| | PLIETNGETGEIVW | | | LTKAERGGLSEL | | | |
| | DKGRDFATVRKVL | | | DKAGFIKRQLVE | | | |
| | SMPQVNIVKKTEV | | | TRQITKHVAQIL | | | |
| | QTGGFSKESILPKR | | | DSRMNTKYDEN | | | |
| | NSDKLIARKKDWD | | | DKLIREVKVITL | | | |
| | PKKYGGFDSPTVA | | | KSKLVSDFRKDF | | | |
| | YSVLVVAKVEKGK | | | QFYKVREINNY | | | |
| | SKKLKSVKELLGITI | | | HHAHDAYLNAV | | | |
| | MERSSFEKNPIDFL | | | VGTALIKKYPKL | | | |
| | EAKGYKEVKKDLII | | | ESEFVYGDYKV | | | |
| | KLPKYSLFELENGR | | | YDVRKMIAKSE | | | |
| | KRMLASAGELQKG | | | QEIGKATAKYFF | | | |
| | NELALPSKYVNFLY | | | YSNIMNFFKTEI | | | |
| | LASHYEKLKGSPED | | | TLANGEIRKRPLI | | | |
| | NEQKQLFVEQHKH | | | ETNGETGEIVW | | | |
| | YLDEIIEQISEFSKR | | | DKGRDFATVRK | | | |
| | VILADANLDKVLSA | | | VLSMPQVNIVK | | | |
| | YNKHRDKPIREQAE | | | KTEVQTGGFSKE | | | |
| | NIIHLFTLTNLGAPA | | | SILPKRNSDKLIA | | | |
| | AFKYFDTTIDRKRY | | | RKKDWDPKKY | | | |
| | TSTKEVLDATLIHQ | | | GGFDSPTVAYSV | | | |
| | SITGLYETRIDLSQL | | | LVVAKVEKGKS | | | |
| | GGD | | | KKLKSVKELLGI | | | |
| | | | | TIMERSSFEKNPI | | | |
| | | | | DFLEAKGYKEV | | | |
| | | | | KKDLIIKLPKYS | | | |
| | | | | LFELENGRKRM | | | |
| | | | | LASAGELQKGN | | | |
| | | | | ELALPSKYVNFL | | | |
| | | | | YLASHYEKLKG | | | |
| | | | | SPEDNEQKQLFV | | | |
| | | | | EQHKHYLDEIIE | | | |
| | | | | QISEFSKRVILAD | | | |
| | | | | ANLDKVLSAYN | | | |
| | | | | KHRDKPIREQAE | | | |
| | | | | NIIHLFTLTNLGA | | | |
| | | | | PAAFKYFDTTID | | | |
| | | | | RKRYTSTKEVL | | | |
| | | | | DATLIHQSITGL | | | |
| | | | | YETRIDLSQLGG | | | |
| | | | | D | | | |
| 8679 | NQKFILGLDIGITSV | N582A | 8688 | NQKFILGLDIGIT | sluCas9 | WP_002460848 | NNGG |
| | GYGLIDYETKNIID | | | SVGYGLIDYETK | | | |
| | AGVRLFPEANVEN | | | NIIDAGVRLFPE | | | |
| | NEGRRSKRGSRRL | | | ANVENNEGRRS | | | |
| | KRRRIHRLERVKKL | | | KRGSRRLKRRRI | | | |
| | LEDYNLLDQSQIPQ | | | HRLERVKKLLE | | | |
| | STNPYAIRVKGLSE | | | DYNLLDQSQIPQ | | | |
| | ALSKDELVIALLHI | | | STNPYAIRVKGL | | | |
| | AKRRGIHKIDVIDS | | | SEALSKDELVIA | | | |
| | NDDVGNELSTKEQ | | | LLHIAKRRGIHKI | | | |
| | LNKNSKLLKDKFV | | | DVIDSNDDVGN | | | |
| | CQIQLERMNEGQV | | | ELSTKEQLNKNS | | | |
| | RGEKNRFKTADIIK | | | KLLKDKFVCQIQ | | | |
| | EIIQLLNVQKNFHQ | | | LERMNEGQVRG | | | |
| | LDENFINKYIELVE | | | EKNRFKTADIIK | | | |
| | MRREYFEGPGKGS | | | EIIQLLNVQKNF | | | |
| | PYGWEGDPKAWY | | | HQLDENFINKYI | | | |
| | ETLMGHCTYFPDEL | | | ELVEMRREYFE | | | |
| | RSVKYAYSADLEN | | | GPGKGSPYGWE | | | |
| | ALNDLNNLVIQRD | | | GDPKAWYETLM | | | |
| | GLSKLEYHEKYHII | | | GHCTYFPDELRS | | | |
| | ENVFKQKKKPTLK | | | VKYAYSADLEN | | | |
| | QIANEINVNPEDIK | | | ALNDLNNLVIQR | | | |
| | GYRITKSGKPQFTE | | | DGLSKLEYHEK | | | |
| | FKLYHDLKSVLFD | | | YHIIENVFKQKK | | | |
| | QSILENEDVLDQIA | | | KPTLKQIANEIN | | | |
| | EILTIYQDKDSIKSK | | | VNPEDIKGYRIT | | | |
| | LTELDILLNEEDKE | | | KSGKPQFTEFKL | | | |
| | NIAQLTGYTGTHRL | | | YHDLKSVLFDQ | | | |
| | SLKCIRLVLEEQWY | | | SILENEDVLDQI | | | |
| | SSRNQMEIFTHLNI | | | AEILTIYQDKDSI | | | |
| | KPKKINLTAANKIP | | | KSKLTELDILLN | | | |
| | KAMIDEFILSPVVK | | | EEDKENIAQLTG | | | |
| | RTFGQAINLINKIIE | | | YTGTHRLSLKCI | | | |
| | KYGVPEDIIIELARE | | | RLVLEEQWYSS | | | |
| | NNSKDKQKFINEM | | | RNQMEIFTHLNI | | | |
| | QKKNENTRKRINEII | | | KPKKINLTAANK | | | |
| | GKYGNQNAKRLVE | | | IPKAMIDEFILSP | | | |
| | KIRLHDEQEGKCLY | | | VVKRTFGQAINL | | | |
| | SLESIPLEDLLNNPN | | | INKIIEKYGVPED | | | |
| | HYEVDHIIPRSVSFD | | | IIIELARENNSKD | | | |
| | NSYHNKVLVKQSE | | | KQKFINEMQKK | | | |
| | NSKKSNLTPYQYFN | | | NENTRKRINEIIG | | | |
| | SGKSKLSYNQFKQ | | | KYGNQNAKRLV | | | |
| | HILNLSKSQDRISK | | | EKIRLHDEQEGK | | | |
| | KKKEYLLEERDINK | | | CLYSLESIPLEDL | | | |
| | FEVQKEFINRNLVD | | | LNNPNHYEVDHI | | | |
| | TRYATRELTNYLK | | | IPRSVSFDNSYH | | | |
| | AYFSANNMNVKVK | | | NKVLVKQSEAS | | | |
| | TINGSFTDYLRKVW | | | KKSNLTPYQYF | | | |
| | KFKKERNHGYKHH | | | NSGKSKLSYNQF | | | |
| | AEDALIIANADFLF | | | KQHILNLSKSQD | | | |
| | KENKKLKAVNSVL | | | RISKKKKEYLLE | | | |
| | EKPEIESKQLDIQV | | | ERDINKFEVQKE | | | |
| | DSEDNYSEMFIIPK | | | FINRNLVDTRYA | | | |
| | QVQDIKDFRNFKYS | | | TRELTNYLKAYF | | | |
| | HRVDKKPNRQLIN | | | SANNMNVKVKT | | | |
| | DTLYSTRKKDNST | | | INGSFTDYLRKV | | | |
| | YIVQTIKDIYAKDN | | | WKFKKERNHGY | | | |
| | TTLKKQFDKSPEKF | | | KHHAEDALIIAN | | | |
| | LMYQHDPRTFEKL | | | ADFLFKENKKL | | | |
| | EVIMKQYANEKNP | | | KAVNSVLEKPEI | | | |
| | LAKYHEETGEYLT | | | ESKQLDIQVDSE | | | |
| | KYSKKNNGPIVKSL | | | DNYSEMFIIPKQ | | | |
| | KYIGNKLGSHLDVT | | | VQDIKDFRNFKY | | | |
| | HQFKSSTKKLVKLS | | | SHRVDKKPNRQ | | | |
| | IKPYRFDVYLTDKG | | | LINDTLYSTRKK | | | |
| | YKFITISYLDVLKK | | | DNSTYIVQTIKDI | | | |
| | DNYYYIPEQKYDK | | | YAKDNTTLKKQ | | | |
| | LKLGKAIDKNAKFI | | | FDKSPEKFLMY | | | |
| | ASFYKNDLIKLDGE | | | QHDPRTFEKLEV | | | |
| | IYKIIGVNSDTRNMI | | | IMKQYANEKNP | | | |
| | ELDLPDIRYKEYCE | | | LAKYHEETGEY | | | |
| | LNNIKGEPRIKKTIG | | | LTKYSKKNNGPI | | | |
| | KKVNSIEKLTTDVL | | | VKSLKYIGNKLG | | | |
| | GNVFTNTQYTKPQ | | | SHLDVTHQFKSS | | | |
| | LLFKRGN | | | TKKLVKLSIKPY | | | |
| | | | | RFDVYLTDKGY | | | |
| | | | | KFITISYLDVLK | | | |
| | | | | KDNYYYIPEQK | | | |
| | | | | YDKLKLGKAID | | | |
| | | | | KNAKFIASFYKN | | | |
| | | | | DLIKLDGEIYKII | | | |
| | | | | GVNSDTRNMIEL | | | |
| | | | | DLPDIRYKEYCE | | | |
| | | | | LNNIKGEPRIKK | | | |
| | | | | TIGKKVNSIEKL | | | |
| | | | | TTDVLGNVFTN | | | |
| | | | | TQYTKPQLLFKR | | | |
| | | | | GN | | | |
| 8680 | KRNYILGLDIGITSV | N580A | 8689 | KRNYILGLDIGIT | saCas9 | J7RUA5 | NNGRRT |
| | GYGIIDYETRDVID | | | SVGYGIIDYETR | | | |
| | AGVRLFKEANVEN | | | DVIDAGVRLFKE | | | |
| | NEGRRSKRGARRL | | | ANVENNEGRRS | | | |
| | KRRRRHRIQRVKK | | | KRGARRLKRRR | | | |
| | LLFDYNLLTDHSEL | | | RHRIQRVKKLLF | | | |
| | SGINPYEARVKGLS | | | DYNLLTDHSELS | | | |
| | QKLSEEEFSAALLH | | | GINPYEARVKGL | | | |
| | LAKRRGVHNVNEV | | | SQKLSEEEFSAA | | | |
| | EEDTGNELSTKEQI | | | LLHLAKRRGVH | | | |
| | SRNSKALEEKYVA | | | NVNEVEEDTGN | | | |
| | ELQLERLKKDGEV | | | ELSTKEQISRNS | | | |
| | RGSINRFKTSDYVK | | | KALEEKYVAEL | | | |
| | EAKQLLKVQKAYH | | | QLERLKKDGEV | | | |
| | QLDQSFIDTYIDLLE | | | RGSINRFKTSDY | | | |
| | TRRTYYEGPGEGSP | | | VKEAKQLLKVQ | | | |
| | FGWKDIKEWYEML | | | KAYHQLDQSFID | | | |
| | MGHCTYFPEELRSV | | | TYIDLLETRRTY | | | |
| | KYAYNADLYNALN | | | YEGPGEGSPFG | | | |
| | DLNNLVITRDENEK | | | WKDIKEWYEML | | | |
| | LEYYEKFQIIENVF | | | MGHCTYFPEEL | | | |
| | KQKKKPTLKQIAKE | | | RSVKYAYNADL | | | |
| | ILVNEEDIKGYRVT | | | YNALNDLNNLV | | | |
| | STGKPEFTNLKVYH | | | ITRDENEKLEYY | | | |
| | DIKDITARKEIIENA | | | EKFQIIENVFKQ | | | |
| | ELLDQIAKILTIYQS | | | KKKPTLKQIAKE | | | |
| | SEDIQEELTNLNSEL | | | ILVNEEDIKGYR | | | |
| | TQEEIEQISNLKGYT | | | VTSTGKPEFTNL | | | |
| | GTHNLSLKAINLIL | | | KVYHDIKDITAR | | | |
| | DELWHTNDNQIAIF | | | KEIIENAELLDQI | | | |
| | NRLKLVPKKVDLS | | | AKILTIYQSSEDI | | | |
| | QQKEIPTTLVDDFIL | | | QEELTNLNSELT | | | |
| | SPVVKRSFIQSIKVI | | | QEEIEQISNLKG | | | |
| | NAIIKKYGLPNDIIIE | | | YTGTHNLSLKAI | | | |
| | LAREKNSKDAQKM | | | NLILDELWHTN | | | |
| | INEMQKRNRQTNE | | | DNQIAIFNRLKL | | | |
| | RIEEIIRTTGKENAK | | | VPKKVDLSQQK | | | |
| | YLIEKIKLHDMQEG | | | EIPTTLVDDFILS | | | |
| | KCLYSLEAIPLEDL | | | PVVKRSFIQSIKV | | | |
| | LNNPFNYEVDHIIP | | | INAIIKKYGLPN | | | |
| | RSVSFDNSFNNKVL | | | DIIIELAREKNSK | | | |
| | VKQEENSKKGNRT | | | DAQKMINEMQK | | | |
| | PFQYLSSSDSKISYE | | | RNRQTNERIEEII | | | |
| | TFKKHILNLAKGKG | | | RTTGKENAKYLI | | | |
| | RISKTKKEYLLEER | | | EKIKLHDMQEG | | | |
| | DINRFSVQKDFINR | | | KCLYSLEAIPLE | | | |
| | NLVDTRYATRGLM | | | DLLNNPFNYEV | | | |
| | NLLRSYFRVNNLD | | | DHIIPRSVSFDNS | | | |
| | VKVKSINGGFTSFL | | | FNNKVLVKQEE | | | |
| | RRKWKFKKERNKG | | | ASKKGNRTPFQ | | | |
| | YKHHAEDALIIANA | | | YLSSSDSKISYET | | | |
| | DFIFKEWKKLDKA | | | FKKHILNLAKGK | | | |
| | KKVMENQMFEEKQ | | | GRISKTKKEYLL | | | |
| | AESMPEIETEQEYK | | | EERDINRFSVQK | | | |
| | EIFITPHQIKHIKDF | | | DFINRNLVDTRY | | | |
| | KDYKYSHRVDKKP | | | ATRGLMNLLRS | | | |
| | NRELINDTLYSTRK | | | YFRVNNLDVKV | | | |
| | DDKGNTLIVNNLN | | | KSINGGFTSFLR | | | |
| | GLYDKDNDKLKKL | | | RKWKFKKERNK | | | |
| | INKSPEKLLMYHHD | | | GYKHHAEDALII | | | |
| | PQTYQKLKLIMEQ | | | ANADFIFKEWK | | | |
| | YGDEKNPLYKYYE | | | KLDKAKKVMEN | | | |
| | ETGNYLTKYSKKD | | | QMFEEKQAESM | | | |
| | NGPVIKKIKYYGNK | | | PEIETEQEYKEIF | | | |
| | LNAHLDITDDYPNS | | | ITPHQIKHIKDFK | | | |
| | RNKVVKLSLKPYR | | | DYKYSHRVDKK | | | |
| | FDVYLDNGVYKFV | | | PNRELINDTLYS | | | |
| | TVKNLDVIKKENY | | | TRKDDKGNTLIV | | | |
| | YEVNSKCYEEAKK | | | NNLNGLYDKDN | | | |
| | LKKISNQAEFIASFY | | | DKLKKLINKSPE | | | |
| | NNDLIKINGELYRV | | | KLLMYHHDPQT | | | |
| | IGVNNDLLNRIEVN | | | YQKLKLIMEQY | | | |
| | MIDITYREYLENMN | | | GDEKNPLYKYY | | | |
| | DKRPPRIIKTIASKT | | | EETGNYLTKYS | | | |
| | QSIKKYSTDILGNL | | | KKDNGPVIKKIK | | | |
| | YEVKSKKHPQIIKK | | | YYGNKLNAHLD | | | |
| | G | | | ITDDYPNSRNKV | | | |
| | | | | VKLSLKPYRFDV | | | |
| | | | | YLDNGVYKFVT | | | |
| | | | | VKNLDVIKKEN | | | |
| | | | | YYEVNSKCYEE | | | |
| | | | | AKKLKKISNQAE | | | |
| | | | | FIASFYNNDLIKI | | | |
| | | | | NGELYRVIGVN | | | |
| | | | | NDLLNRIEVNMI | | | |
| | | | | DITYREYLENM | | | |
| | | | | NDKRPPRIIKTIA | | | |
| | | | | SKTQSIKKYSTDI | | | |
| | | | | LGNLYEVKSKK | | | |
| | | | | HPQIIKKG | | | |
| 8681 | DKKYSIGLDIGTNS | H840A | 8690 | DKKYSIGLDIGT | NGCas9 | NA | NGN |
| | VGWAVITDEYKVP | | | NSVGWAVITDE | | | |
| | SKKFKVLGNTDRH | | | YKVPSKKFKVL | | | |
| | SIKKNLIGALLFDSG | | | GNTDRHSIKKNL | | | |
| | ETAEATRLKRTARR | | | IGALLFDSGETA | | | |
| | RYTRRKNRICYLQE | | | EATRLKRTARR | | | |
| | IFSNEMAKVDDSFF | | | RYTRRKNRICYL | | | |
| | HRLEESFLVEEDKK | | | QEIFSNEMAKVD | | | |
| | HERHPIFGNIVDEV | | | DSFFHRLEESFL | | | |
| | AYHEKYPTIYHLRK | | | VEEDKKHERHPI | | | |
| | KLVDSTDKADLRLI | | | FGNIVDEVAYHE | | | |
| | YLALAHMIKFRGH | | | KYPTIYHLRKKL | | | |
| | FLIEGDLNPDNSDV | | | VDSTDKADLRLI | | | |
| | DKLFIQLVQTYNQL | | | YLALAHMIKFR | | | |
| | FEENPINASGVDAK | | | GHFLIEGDLNPD | | | |
| | AILSARLSKSRRLE | | | NSDVDKLFIQLV | | | |
| | NLIAQLPGEKKNGL | | | QTYNQLFEENPI | | | |
| | FGNLIALSLGLTPNF | | | NASGVDAKAILS | | | |
| | KSNFDLAEDAKLQ | | | ARLSKSRRLENL | | | |
| | LSKDTYDDDLDNL | | | IAQLPGEKKNGL | | | |
| | LAQIGDQYADLFLA | | | FGNLIALSLGLT | | | |
| | AKNLSDAILLSDILR | | | PNFKSNFDLAED | | | |
| | VNTEITKAPLSASM | | | AKLQLSKDTYD | | | |
| | IKRYDEHHQDLTLL | | | DDLDNLLAQIG | | | |
| | KALVRQQLPEKYK | | | DQYADLFLAAK | | | |
| | EIFFDQSKNGYAGY | | | NLSDAILLSDILR | | | |
| | IDGGASQEEFYKFI | | | VNTEITKAPLSA | | | |
| | KPILEKMDGTEELL | | | SMIKRYDEHHQ | | | |
| | VKLNREDLLRKQR | | | DLTLLKALVRQ | | | |
| | TFDNGSIPHQIHLGE | | | QLPEKYKEIFFD | | | |
| | LHAILRRQEDFYPF | | | QSKNGYAGYID | | | |
| | LKDNREKIEKILTFR | | | GGASQEEFYKFI | | | |
| | IPYYVGPLARGNSR | | | KPILEKMDGTEE | | | |
| | FAWMTRKSEETITP | | | LLVKLNREDLLR | | | |
| | WNFEEVVDKGASA | | | KQRTFDNGSIPH | | | |
| | QSFIERMTNFDKNL | | | QIHLGELHAILR | | | |
| | PNEKVLPKHSLLYE | | | RQEDFYPFLKDN | | | |
| | YFTVYNELTKVKY | | | REKIEKILTFRIP | | | |
| | VTEGMRKPAFLSG | | | YYVGPLARGNS | | | |
| | EQKKAIVDLLFKTN | | | RFAWMTRKSEE | | | |
| | RKVTVKQLKEDYF | | | TITPWNFEEVVD | | | |
| | KKIECFDSVEISGVE | | | KGASAQSFIERM | | | |
| | DRFNASLGTYHDL | | | TNFDKNLPNEK | | | |
| | LKIIKDKDFLDNEE | | | VLPKHSLLYEYF | | | |
| | NEDILEDIVLTLTLF | | | TVYNELTKVKY | | | |
| | EDREMIEERLKTYA | | | VTEGMRKPAFL | | | |
| | HLFDDKVMKQLKR | | | SGEQKKAIVDLL | | | |
| | RRYTGWGRLSRKLI | | | FKTNRKVTVKQ | | | |
| | NGIRDKQSGKTILD | | | LKEDYFKKIECF | | | |
| | FLKSDGFANRNFM | | | DSVEISGVEDRF | | | |
| | QLIHDDSLTFKEDI | | | NASLGTYHDLL | | | |
| | QKAQVSGQGDSLH | | | KIIKDKDFLDNE | | | |
| | EHIANLAGSPAIKK | | | ENEDILEDIVLTL | | | |
| | GILQTVKVVDELV | | | TLFEDREMIEER | | | |
| | KVMGRHKPENIVIE | | | LKTYAHLFDDK | | | |
| | MARENQTTQKGQK | | | VMKQLKRRRYT | | | |
| | NSRERMKRIEEGIK | | | GWGRLSRKLIN | | | |
| | ELGSQILKEHPVEN | | | GIRDKQSGKTIL | | | |
| | TQLQNEKLYLYYL | | | DFLKSDGFANR | | | |
| | QNGRDMYVDQEL | | | NFMQLIHDDSLT | | | |
| | DINRLSDYDVDHIV | | | FKEDIQKAQVSG | | | |
| | PQSFLKDDSIDNKV | | | QGDSLHEHIANL | | | |
| | LTRSDKNRGKSDN | | | AGSPAIKKGILQ | | | |
| | VPSEEVVKKMKNY | | | TVKVVDELVKV | | | |
| | WRQLLNAKLITQR | | | MGRHKPENIVIE | | | |
| | KFDNLTKAERGGL | | | MARENQTTQKG | | | |
| | SELDKAGFIKRQLV | | | QKNSRERMKRIE | | | |
| | ETRQITKHVAQILD | | | EGIKELGSQILKE | | | |
| | SRMNTKYDENDKL | | | HPVENTQLQNE | | | |
| | IREVKVITLKSKLVS | | | KLYLYYLQNGR | | | |
| | DFRKDFQFYKVREI | | | DMYVDQELDIN | | | |
| | NNYHHAHDAYLN | | | RLSDYDVDAIVP | | | |
| | AVVGTALIKKYPKL | | | QSFLKDDSIDNK | | | |
| | ESEFVYGDYKVYD | | | VLTRSDKNRGK | | | |
| | VRKMIAKSEQEIGK | | | SDNVPSEEVVK | | | |
| | ATAKYFFYSNIMNF | | | KMKNYWRQLL | | | |
| | FKTEITLANGEIRKR | | | NAKLITQRKFDN | | | |
| | PLIETNGETGEIVW | | | LTKAERGGLSEL | | | |
| | DKGRDFATVRKVL | | | DKAGFIKRQLVE | | | |
| | SMPQVNIVKKTEV | | | TRQITKHVAQIL | | | |
| | QTGGFSKESIRPKR | | | DSRMNTKYDEN | | | |
| | NSDKLIARKKDWD | | | DKLIREVKVITL | | | |
| | PKKYGGFVSPTVA | | | KSKLVSDFRKDF | | | |
| | YSVLVVAKVEKGK | | | QFYKVREINNY | | | |
| | SKKLKSVKELLGITI | | | HHAHDAYLNAV | | | |
| | MERSSFEKNPIDFL | | | VGTALIKKYPKL | | | |
| | EAKGYKEVKKDLII | | | ESEFVYGDYKV | | | |
| | KLPKYSLFELENGR | | | YDVRKMIAKSE | | | |
| | KRMLASARFLQKG | | | QEIGKATAKYFF | | | |
| | NELALPSKYVNFLY | | | YSNIMNFFKTEI | | | |
| | LASHYEKLKGSPED | | | TLANGEIRKRPLI | | | |
| | NEQKQLFVEQHKH | | | ETNGETGEIVW | | | |
| | YLDEIIEQISEFSKR | | | DKGRDFATVRK | | | |
| | VILADANLDKVLSA | | | VLSMPQVNIVK | | | |
| | YNKHRDKPIREQAE | | | KTEVQTGGFSKE | | | |
| | NIIHLFTLTNLGAPR | | | SIRPKRNSDKLIA | | | |
| | AFKYFDTTIDRKVY | | | RKKDWDPKKY | | | |
| | RSTKEVLDATLIHQ | | | GGFVSPTVAYSV | | | |
| | SITGLYETRIDLSQL | | | LVVAKVEKGKS | | | |
| | GGD | | | KKLKSVKELLGI | | | |
| | | | | TIMERSSFEKNPI | | | |
| | | | | DFLEAKGYKEV | | | |
| | | | | KKDLIIKLPKYS | | | |
| | | | | LFELENGRKRM | | | |
| | | | | LASARFLQKGN | | | |
| | | | | ELALPSKYVNFL | | | |
| | | | | YLASHYEKLKG | | | |
| | | | | SPEDNEQKQLFV | | | |
| | | | | EQHKHYLDEIIE | | | |
| | | | | QISEFSKRVILAD | | | |
| | | | | ANLDKVLSAYN | | | |
| | | | | KHRDKPIREQAE | | | |
| | | | | NIIHLFTLTNLGA | | | |
| | | | | PRAFKYFDTTID | | | |
| | | | | RKVYRSTKEVL | | | |
| | | | | DATLIHQSITGL | | | |
| | | | | YETRIDLSQLGG | | | |
| | | | | D | | | |
| 8682 | ATRSFILKIEPNEEV | NA | | NA | Cas12b | WP_095142515 | TTTA |
| | KKGLWKTHEVLNH | | | | | | |
| | GIAYYMNILKLIRQ | | | | | | |
| | EAIYEHHEQDPKNP | | | | | | |
| | KKVSKAEIQAELW | | | | | | |
| | DFVLKMQKCNSFT | | | | | | |
| | HEVDKDEVFNILRE | | | | | | |
| | LYEELVPSSVEKKG | | | | | | |
| | EANQLSNKFLYPLV | | | | | | |
| | DPNSQSGKGTASSG | | | | | | |
| | RKPRWYNIKIAGD | | | | | | |
| | PSWEEEKKKWEED | | | | | | |
| | KKKDPLAKILGKLA | | | | | | |
| | EYGLIPLFIPYTDSN | | | | | | |
| | EPIVKEIKWMEKSR | | | | | | |
| | NQSVRRLDKDMFI | | | | | | |
| | QALERFLSWESWN | | | | | | |
| | LKVKEEYEKVEKE | | | | | | |
| | YKTLEERIKEDIQA | | | | | | |
| | LKALEQYEKERQE | | | | | | |
| | QLLRDTLNTNEYRL | | | | | | |
| | SKRGLRGWREIIQK | | | | | | |
| | WLKMDENEPSEKY | | | | | | |
| | LEVFKDYQRKHPR | | | | | | |
| | EAGDYSVYEFLSK | | | | | | |
| | KENHFIWRNHPEYP | | | | | | |
| | YLYATFCEIDKKKK | | | | | | |
| | DAKQQATFTLADPI | | | | | | |
| | NHPLWVRFEERSGS | | | | | | |
| | NLNKYRILTEQLHT | | | | | | |
| | EKLKKKLTVQLDR | | | | | | |
| | LIYPTESGGWEEKG | | | | | | |
| | KVDIVLLPSRQFYN | | | | | | |
| | QIFLDIEEKGKHAF | | | | | | |
| | TYKDESIKFPLKGT | | | | | | |
| | LGGARVQFDRDHL | | | | | | |
| | RRYPHKVESGNVG | | | | | | |
| | RIYFNMTVNIEPTES | | | | | | |
| | PVSKSLKIHRDDFP | | | | | | |
| | KVVNFKPKELTEWI | | | | | | |
| | KDSKGKKLKSGIES | | | | | | |
| | LEIGLRVMSIDLGQ | | | | | | |
| | RQAAAASIFEVVDQ | | | | | | |
| | KPDIEGKLFFPIKGT | | | | | | |
| | ELYAVHRASFNIKL | | | | | | |
| | PGETLVKSREVLRK | | | | | | |
| | AREDNLKLMNQKL | | | | | | |
| | NFLRNVLHFQQFED | | | | | | |
| | ITEREKRVTKWISR | | | | | | |
| | QENSDVPLVYQDE | | | | | | |
| | LIQIRELMYKPYKD | | | | | | |
| | WVAFLKQLHKRLE | | | | | | |
| | VEIGKEVKHWRKS | | | | | | |
| | LSDGRKGLYGISLK | | | | | | |
| | NIDEIDRTRKFLLR | | | | | | |
| | WSLRPTEPGEVRRL | | | | | | |
| | EPGQRFAIDQLNHL | | | | | | |
| | NALKEDRLKKMAN | | | | | | |
| | TIIMHALGYCYDVR | | | | | | |
| | KKKWQAKNPACQI | | | | | | |
| | ILFEDLSNYNPYEE | | | | | | |
| | RSRFENSKLMKWS | | | | | | |
| | RREIPRQVALQGEI | | | | | | |
| | YGLQVGEVGAQFS | | | | | | |
| | SRFHAKTGSPGIRC | | | | | | |
| | SVVTKEKLQDNRFF | | | | | | |
| | KNLQREGRLTLDKI | | | | | | |
| | AVLKEGDLYPDKG | | | | | | |
| | GEKFISLSKDRKCV | | | | | | |
| | TTHADINAAQNLQ | | | | | | |
| | KRFWTRTHGFYKV | | | | | | |
| | YCKAYQVDGQTVY | | | | | | |
| | IPESKDQKQKIIEEF | | | | | | |
| | GEGYFILKDGVYE | | | | | | |
| | WVNAGKLKIKKGS | | | | | | |
| | SKQSSSELVDSDIL | | | | | | |
| | KDSFDLASELKGEK | | | | | | |
| | LMLYRDPSGNVFPS | | | | | | |
| | DKWMAAGVFFGK | | | | | | |
| | LERILISKLTNQYSI | | | | | | |
| | STIEDDSSKQSM | | | | | | |
| 8683 | DKKYSIGLDIGTNS | H840A | 8691 | DKKYSIGLDIGT | VRQR | NA | NGA |
| | VGWAVITDEYKVP | | | NSVGWAVITDE | | | |
| | SKKFKVLGNTDRH | | | YKVPSKKFKVL | | | |
| | SIKKNLIGALLFDSG | | | GNTDRHSIKKNL | | | |
| | ETAEATRLKRTARR | | | IGALLFDSGETA | | | |
| | RYTRRKNRICYLQE | | | EATRLKRTARR | | | |
| | IFSNEMAKVDDSFF | | | RYTRRKNRICYL | | | |
| | HRLEESFLVEEDKK | | | QEIFSNEMAKVD | | | |
| | HERHPIFGNIVDEV | | | DSFFHRLEESFL | | | |
| | AYHEKYPTIYHLRK | | | VEEDKKHERHPI | | | |
| | KLVDSTDKADLRLI | | | FGNIVDEVAYHE | | | |
| | YLALAHMIKFRGH | | | KYPTIYHLRKKL | | | |
| | FLIEGDLNPDNSDV | | | VDSTDKADLRLI | | | |
| | DKLFIQLVQTYNQL | | | YLALAHMIKFR | | | |
| | FEENPINASGVDAK | | | GHFLIEGDLNPD | | | |
| | AILSARLSKSRRLE | | | NSDVDKLFIQLV | | | |
| | NLIAQLPGEKKNGL | | | QTYNQLFEENPI | | | |
| | FGNLIALSLGLTPNF | | | NASGVDAKAILS | | | |
| | KSNFDLAEDAKLQ | | | ARLSKSRRLENL | | | |
| | LSKDTYDDDLDNL | | | IAQLPGEKKNGL | | | |
| | LAQIGDQYADLFLA | | | FGNLIALSLGLT | | | |
| | AKNLSDAILLSDILR | | | PNFKSNFDLAED | | | |
| | VNTEITKAPLSASM | | | AKLQLSKDTYD | | | |
| | IKRYDEHHQDLTLL | | | DDLDNLLAQIG | | | |
| | KALVRQQLPEKYK | | | DQYADLFLAAK | | | |
| | EIFFDQSKNGYAGY | | | NLSDAILLSDILR | | | |
| | IDGGASQEEFYKFI | | | VNTEITKAPLSA | | | |
| | KPILEKMDGTEELL | | | SMIKRYDEHHQ | | | |
| | VKLNREDLLRKQR | | | DLTLLKALVRQ | | | |
| | TFDNGSIPHQIHLGE | | | QLPEKYKEIFFD | | | |
| | LHAILRRQEDFYPF | | | QSKNGYAGYID | | | |
| | LKDNREKIEKILTFR | | | GGASQEEFYKFI | | | |
| | IPYYVGPLARGNSR | | | KPILEKMDGTEE | | | |
| | FAWMTRKSEETITP | | | LLVKLNREDLLR | | | |
| | WNFEEVVDKGASA | | | KQRTFDNGSIPH | | | |
| | QSFIERMTNFDKNL | | | QIHLGELHAILR | | | |
| | PNEKVLPKHSLLYE | | | RQEDFYPFLKDN | | | |
| | YFTVYNELTKVKY | | | REKIEKILTFRIP | | | |
| | VTEGMRKPAFLSG | | | YYVGPLARGNS | | | |
| | EQKKAIVDLLFKTN | | | RFAWMTRKSEE | | | |
| | RKVTVKQLKEDYF | | | TITPWNFEEVVD | | | |
| | KKIECFDSVEISGVE | | | KGASAQSFIERM | | | |
| | DRFNASLGTYHDL | | | TNFDKNLPNEK | | | |
| | LKIIKDKDFLDNEE | | | VLPKHSLLYEYF | | | |
| | NEDILEDIVLTLTLF | | | TVYNELTKVKY | | | |
| | EDREMIEERLKTYA | | | VTEGMRKPAFL | | | |
| | HLFDDKVMKQLKR | | | SGEQKKAIVDLL | | | |
| | RRYTGWGRLSRKLI | | | FKTNRKVTVKQ | | | |
| | NGIRDKQSGKTILD | | | LKEDYFKKIECF | | | |
| | FLKSDGFANRNFM | | | DSVEISGVEDRF | | | |
| | QLIHDDSLTFKEDI | | | NASLGTYHDLL | | | |
| | QKAQVSGQGDSLH | | | KIIKDKDFLDNE | | | |
| | EHIANLAGSPAIKK | | | ENEDILEDIVLTL | | | |
| | GILQTVKVVDELV | | | TLFEDREMIEER | | | |
| | KVMGRHKPENIVIE | | | LKTYAHLFDDK | | | |
| | MARENQTTQKGQK | | | VMKQLKRRRYT | | | |
| | NSRERMKRIEEGIK | | | GWGRLSRKLIN | | | |
| | ELGSQILKEHPVEN | | | GIRDKQSGKTIL | | | |
| | TQLQNEKLYLYYL | | | DFLKSDGFANR | | | |
| | QNGRDMYVDQEL | | | NFMQLIHDDSLT | | | |
| | DINRLSDYDVDHIV | | | FKEDIQKAQVSG | | | |
| | PQSFLKDDSIDNKV | | | QGDSLHEHIANL | | | |
| | LTRSDKNRGKSDN | | | AGSPAIKKGILQ | | | |
| | VPSEEVVKKMKNY | | | TVKVVDELVKV | | | |
| | WRQLLNAKLITQR | | | MGRHKPENIVIE | | | |
| | KFDNLTKAERGGL | | | MARENQTTQKG | | | |
| | SELDKAGFIKRQLV | | | QKNSRERMKRIE | | | |
| | ETRQITKHVAQILD | | | EGIKELGSQILKE | | | |
| | SRMNTKYDENDKL | | | HPVENTQLQNE | | | |
| | IREVKVITLKSKLVS | | | KLYLYYLQNGR | | | |
| | DFRKDFQFYKVREI | | | DMYVDQELDIN | | | |
| | NNYHHAHDAYLN | | | RLSDYDVDAIVP | | | |
| | AVVGTALIKKYPKL | | | QSFLKDDSIDNK | | | |
| | ESEFVYGDYKVYD | | | VLTRSDKNRGK | | | |
| | VRKMIAKSEQEIGK | | | SDNVPSEEVVK | | | |
| | ATAKYFFYSNIMNF | | | KMKNYWRQLL | | | |
| | FKTEITLANGEIRKR | | | NAKLITQRKFDN | | | |
| | PLIETNGETGEIVW | | | LTKAERGGLSEL | | | |
| | DKGRDFATVRKVL | | | DKAGFIKRQLVE | | | |
| | SMPQVNIVKKTEV | | | TRQITKHVAQIL | | | |
| | QTGGFSKESILPKR | | | DSRMNTKYDEN | | | |
| | NSDKLIARKKDWD | | | DKLIREVKVITL | | | |
| | PKKYGGFVSPTVA | | | KSKLVSDFRKDF | | | |
| | YSVLVVAKVEKGK | | | QFYKVREINNY | | | |
| | SKKLKSVKELLGITI | | | HHAHDAYLNAV | | | |
| | MERSSFEKNPIDFL | | | VGTALIKKYPKL | | | |
| | EAKGYKEVKKDLII | | | ESEFVYGDYKV | | | |
| | KLPKYSLFELENGR | | | YDVRKMIAKSE | | | |
| | KRMLASARELQKG | | | QEIGKATAKYFF | | | |
| | NELALPSKYVNFLY | | | YSNIMNFFKTEI | | | |
| | LASHYEKLKGSPED | | | TLANGEIRKRPLI | | | |
| | NEQKQLFVEQHKH | | | ETNGETGEIVW | | | |
| | YLDEIIEQISEFSKR | | | DKGRDFATVRK | | | |
| | VILADANLDKVLSA | | | VLSMPQVNIVK | | | |
| | YNKHRDKPIREQAE | | | KTEVQTGGFSKE | | | |
| | NIIHLFTLTNLGAPA | | | SILPKRNSDKLIA | | | |
| | AFKYFDTTIDRKQY | | | RKKDWDPKKY | | | |
| | RSTKEVLDATLIHQ | | | GGFVSPTVAYSV | | | |
| | SITGLYETRIDLSQL | | | LVVAKVEKGKS | | | |
| | GGD | | | KKLKSVKELLGI | | | |
| | | | | TIMERSSFEKNPI | | | |
| | | | | DFLEAKGYKEV | | | |
| | | | | KKDLIIKLPKYS | | | |
| | | | | LFELENGRKRM | | | |
| | | | | LASARELQKGN | | | |
| | | | | ELALPSKYVNFL | | | |
| | | | | YLASHYEKLKG | | | |
| | | | | SPEDNEQKQLFV | | | |
| | | | | EQHKHYLDEIIE | | | |
| | | | | QISEFSKRVILAD | | | |
| | | | | ANLDKVLSAYN | | | |
| | | | | KHRDKPIREQAE | | | |
| | | | | NIIHLFTLTNLGA | | | |
| | | | | PAAFKYFDTTID | | | |
| | | | | RKQYRSTKEVL | | | |
| | | | | DATLIHQSITGL | | | |
| | | | | YETRIDLSQLGG | | | |
| | | | | D | | | |
| 8684 | DKKYSIGLDIGTNS | H840A | 8692 | DKKYSIGLDIGT | SpRY | NA | NRN |
| | VGWAVITDEYKVP | | | NSVGWAVITDE | | | |
| | SKKFKVLGNTDRH | | | YKVPSKKFKVL | | | |
| | SIKKNLIGALLFDSG | | | GNTDRHSIKKNL | | | |
| | ETAERTRLKRTARR | | | IGALLFDSGETA | | | |
| | RYTRRKNRICYLQE | | | ERTRLKRTARRR | | | |
| | IFSNEMAKVDDSFF | | | YTRRKNRICYLQ | | | |
| | HRLEESFLVEEDKK | | | EIFSNEMAKVDD | | | |
| | HERHPIFGNIVDEV | | | SFFHRLEESFLV | | | |
| | AYHEKYPTIYHLRK | | | EEDKKHERHPIF | | | |
| | KLVDSTDKADLRLI | | | GNIVDEVAYHE | | | |
| | YLALAHMIKFRGH | | | KYPTIYHLRKKL | | | |
| | FLIEGDLNPDNSDV | | | VDSTDKADLRLI | | | |
| | DKLFIQLVQTYNQL | | | YLALAHMIKFR | | | |
| | FEENPINASGVDAK | | | GHFLIEGDLNPD | | | |
| | AILSARLSKSRRLE | | | NSDVDKLFIQLV | | | |
| | NLIAQLPGEKKNGL | | | QTYNQLFEENPI | | | |
| | FGNLIALSLGLTPNF | | | NASGVDAKAILS | | | |
| | KSNFDLAEDAKLQ | | | ARLSKSRRLENL | | | |
| | LSKDTYDDDLDNL | | | IAQLPGEKKNGL | | | |
| | LAQIGDQYADLFLA | | | FGNLIALSLGLT | | | |
| | AKNLSDAILLSDILR | | | PNFKSNFDLAED | | | |
| | VNTEITKAPLSASM | | | AKLQLSKDTYD | | | |
| | IKRYDEHHQDLTLL | | | DDLDNLLAQIG | | | |
| | KALVRQQLPEKYK | | | DQYADLFLAAK | | | |
| | EIFFDQSKNGYAGY | | | NLSDAILLSDILR | | | |
| | IDGGASQEEFYKFI | | | VNTEITKAPLSA | | | |
| | KPILEKMDGTEELL | | | SMIKRYDEHHQ | | | |
| | VKLNREDLLRKQR | | | DLTLLKALVRQ | | | |
| | TFDNGSIPHQIHLGE | | | QLPEKYKEIFFD | | | |
| | LHAILRRQEDFYPF | | | QSKNGYAGYID | | | |
| | LKDNREKIEKILTFR | | | GGASQEEFYKFI | | | |
| | IPYYVGPLARGNSR | | | KPILEKMDGTEE | | | |
| | FAWMTRKSEETITP | | | LLVKLNREDLLR | | | |
| | WNFEEVVDKGASA | | | KQRTFDNGSIPH | | | |
| | QSFIERMTNFDKNL | | | QIHLGELHAILR | | | |
| | PNEKVLPKHSLLYE | | | RQEDFYPFLKDN | | | |
| | YFTVYNELTKVKY | | | REKIEKILTFRIP | | | |
| | VTEGMRKPAFLSG | | | YYVGPLARGNS | | | |
| | EQKKAIVDLLFKTN | | | RFAWMTRKSEE | | | |
| | RKVTVKQLKEDYF | | | TITPWNFEEVVD | | | |
| | KKIECFDSVEISGVE | | | KGASAQSFIERM | | | |
| | DRFNASLGTYHDL | | | TNFDKNLPNEK | | | |
| | LKIIKDKDFLDNEE | | | VLPKHSLLYEYF | | | |
| | EDREMIEERLKTYA | | | VTEGMRKPAFL | | | |
| | HLFDDKVMKQLKR | | | SGEQKKAIVDLL | | | |
| | RRYTGWGRLSRKLI | | | FKTNRKVTVKQ | | | |
| | NGIRDKQSGKTILD | | | LKEDYFKKIECF | | | |
| | FLKSDGFANRNFM | | | DSVEISGVEDRF | | | |
| | QLIHDDSLTFKEDI | | | NASLGTYHDLL | | | |
| | QKAQVSGQGDSLH | | | KIIKDKDFLDNE | | | |
| | EHIANLAGSPAIKK | | | ENEDILEDIVLTL | | | |
| | GILQTVKVVDELV | | | TLFEDREMIEER | | | |
| | KVMGRHKPENIVIE | | | LKTYAHLFDDK | | | |
| | MARENQTTQKGQK | | | VMKQLKRRRYT | | | |
| | NSRERMKRIEEGIK | | | GWGRLSRKLIN | | | |
| | ELGSQILKEHPVEN | | | GIRDKQSGKTIL | | | |
| | TQLQNEKLYLYYL | | | DFLKSDGFANR | | | |
| | QNGRDMYVDQEL | | | NFMQLIHDDSLT | | | |
| | DINRLSDYDVDHIV | | | FKEDIQKAQVSG | | | |
| | PQSFLKDDSIDNKV | | | QGDSLHEHIANL | | | |
| | LTRSDKNRGKSDN | | | AGSPAIKKGILQ | | | |
| | VPSEEVVKKMKNY | | | TVKVVDELVKV | | | |
| | WRQLLNAKLITQR | | | MGRHKPENIVIE | | | |
| | KFDNLTKAERGGL | | | MARENQTTQKG | | | |
| | SELDKAGFIKRQLV | | | QKNSRERMKRIE | | | |
| | ETRQITKHVAQILD | | | EGIKELGSQILKE | | | |
| | SRMNTKYDENDKL | | | HPVENTQLQNE | | | |
| | IREVKVITLKSKLVS | | | KLYLYYLQNGR | | | |
| | DFRKDFQFYKVREI | | | DMYVDQELDIN | | | |
| | NNYHHAHDAYLN | | | RLSDYDVDAIVP | | | |
| | AVVGTALIKKYPKL | | | QSFLKDDSIDNK | | | |
| | ESEFVYGDYKVYD | | | VLTRSDKNRGK | | | |
| | VRKMIAKSEQEIGK | | | SDNVPSEEVVK | | | |
| | ATAKYFFYSNIMNF | | | KMKNYWRQLL | | | |
| | FKTEITLANGEIRKR | | | NAKLITQRKFDN | | | |
| | PLIETNGETGEIVW | | | LTKAERGGLSEL | | | |
| | DKGRDFATVRKVL | | | DKAGFIKRQLVE | | | |
| | SMPQVNIVKKTEV | | | TRQITKHVAQIL | | | |
| | QTGGFSKESIRPKR | | | DSRMNTKYDEN | | | |
| | NSDKLIARKKDWD | | | DKLIREVKVITL | | | |
| | PKKYGGFLWPTVA | | | KSKLVSDFRKDF | | | |
| | YSVLVVAKVEKGK | | | QFYKVREINNY | | | |
| | SKKLKSVKELLGITI | | | HHAHDAYLNAV | | | |
| | MERSSFEKNPIDFL | | | VGTALIKKYPKL | | | |
| | EAKGYKEVKKDLII | | | ESEFVYGDYKV | | | |
| | KLPKYSLFELENGR | | | YDVRKMIAKSE | | | |
| | KRMLASAKQLQKG | | | QEIGKATAKYFF | | | |
| | NELALPSKYVNFLY | | | YSNIMNFFKTEI | | | |
| | LASHYEKLKGSPED | | | TLANGEIRKRPLI | | | |
| | NEQKQLFVEQHKH | | | ETNGETGEIVW | | | |
| | YLDEIIEQISEFSKR | | | DKGRDFATVRK | | | |
| | VILADANLDKVLSA | | | VLSMPQVNIVK | | | |
| | YNKHRDKPIREQAE | | | KTEVQTGGFSKE | | | |
| | NIIHLFTLTRLGAPR | | | SIRPKRNSDKLIA | | | |
| | AFKYFDTTIDPKQY | | | RKKDWDPKKY | | | |
| | RSTKEVLDATLIHQ | | | GGFLWPTVAYS | | | |
| | SITGLYETRIDLSQL | | | VLVVAKVEKGK | | | |
| | GGD | | | SKKLKSVKELLG | | | |
| | | | | ITIMERSSFEKNP | | | |
| | | | | IDFLEAKGYKEV | | | |
| | | | | KKDLIIKLPKYS | | | |
| | | | | LFELENGRKRM | | | |
| | | | | LASAKQLQKGN | | | |
| | | | | ELALPSKYVNFL | | | |
| | | | | YLASHYEKLKG | | | |
| | | | | SPEDNEQKQLFV | | | |
| | | | | EQHKHYLDEIIE | | | |
| | | | | QISEFSKRVILAD | | | |
| | | | | ANLDKVLSAYN | | | |
| | | | | KHRDKPIREQAE | | | |
| | | | | NIIHLFTLTRLGA | | | |
| | | | | PRAFKYFDTTID | | | |
| | | | | PKQYRSTKEVL | | | |
| | | | | DATLIHQSITGL | | | |
| | | | | YETRIDLSQLGG | | | |
| | | | | D | | | |
| 8685 | NQKFILGLDIGITSV | N585A | 8693 | NQKFILGLDIGIT | SRGN3.1 | NA | NNGG |
| | GYGLIDYETKNIID | | | SVGYGLIDYETK | | | |
| | AGVRLFPEANVEN | | | NIIDAGVRLFPE | | | |
| | NEGRRSKRGSRRL | | | ANVENNEGRRS | | | |
| | KRRRIHRLERVKLL | | | KRGSRRLKRRRI | | | |
| | LTEYDLINKEQIPTS | | | HRLERVKLLLTE | | | |
| | NNPYQIRVKGLSEI | | | YDLINKEQIPTS | | | |
| | LSKDELAIALLHLA | | | NNPYQIRVKGLS | | | |
| | KRRGIHNVDVAAD | | | EILSKDELAIALL | | | |
| | KEETASDSLSTKDQ | | | HLAKRRGIHNV | | | |
| | INKNAKFLESRYVC | | | DVAADKEETAS | | | |
| | ELQKERLENEGHV | | | DSLSTKDQINKN | | | |
| | RGVENRFLTKDIVR | | | AKFLESRYVCEL | | | |
| | EAKKIIDTQMQYYP | | | QKERLENEGHV | | | |
| | EIDETFKEKYISLVE | | | RGVENRFLTKDI | | | |
| | TRREYFEGPGQGSP | | | VREAKKIIDTQM | | | |
| | FGWNGDLKKWYE | | | QYYPEIDETFKE | | | |
| | MLMGHCTYFPQEL | | | KYISLVETRREY | | | |
| | RSVKYAYSADLEN | | | FEGPGQGSPFG | | | |
| | ALNDLNNLIIQRDN | | | WNGDLKKWYE | | | |
| | SEKLEYHEKYHIIE | | | MLMGHCTYFPQ | | | |
| | NVFKQKKKPTLKQI | | | ELRSVKYAYSA | | | |
| | AKEIGVNPEDIKGY | | | DLFNALNDLNN | | | |
| | RITKSGTPEFTSFKL | | | LIIQRDNSEKLE | | | |
| | FHDLKKVVKDHAI | | | YHEKYHIIENVF | | | |
| | LDDIDLLNQIAEILT | | | KQKKKPTLKQIA | | | |
| | IYQDKDSIVAELGQ | | | KEIGVNPEDIKG | | | |
| | LEYLMSEADKQSIS | | | YRITKSGTPEFTS | | | |
| | ELTGYTGTHSLSLK | | | FKLFHDLKKVV | | | |
| | CMNMIIDELWHSS | | | KDHAILDDIDLL | | | |
| | MNQMEVFTYLNM | | | NQIAEILTIYQDK | | | |
| | RPKKYELKGYQRIP | | | DSIVAELGQLEY | | | |
| | TDMIDDAILSPVVK | | | LMSEADKQSISE | | | |
| | RTFIQSINVINKVIE | | | LTGYTGTHSLSL | | | |
| | KYGIPEDIIIELARE | | | KCMNMIIDELW | | | |
| | NNSDDRKKFINNLQ | | | HSSMNQMEVFT | | | |
| | KKNEATRKRINEIIG | | | YLNMRPKKYEL | | | |
| | QTGNQNAKRIVEKI | | | KGYQRIPTDMID | | | |
| | RLHDQQEGKCLYS | | | DAILSPVVKRTFI | | | |
| | LESIPLEDLLNNPN | | | QSINVINKVIEK | | | |
| | HYEVDHIIPRSVSFD | | | YGIPEDIIIELARE | | | |
| | NSYHNKVLVKQSE | | | NNSDDRKKFINN | | | |
| | NSKKSNLTPYQYFN | | | LQKKNEATRKRI | | | |
| | SGKSKLSYNQFKQ | | | NEIIGQTGNQNA | | | |
| | HILNLSKSQDRISK | | | KRIVEKIRLHDQ | | | |
| | KKKEYLLEERDINK | | | QEGKCLYSLESI | | | |
| | FEVQKEFINRNLVD | | | PLEDLLNNPNHY | | | |
| | TRYATRELTNYLK | | | EVDHIIPRSVSFD | | | |
| | AYFSANNMNVKVK | | | NSYHNKVLVKQ | | | |
| | TINGSFTDYLRKVW | | | SEASKKSNLTPY | | | |
| | KFKKERNHGYKHH | | | QYFNSGKSKLSY | | | |
| | AEDALIIANADFLF | | | NQFKQHILNLSK | | | |
| | KENKKLKAVNSVL | | | SQDRISKKKKEY | | | |
| | EKPEIETKQLDIQV | | | LLEERDINKFEV | | | |
| | DSEDNYSEMFIIPK | | | QKEFINRNLVDT | | | |
| | QVQDIKDFRNFKYS | | | RYATRELTNYL | | | |
| | HRVDKKPNRQLIN | | | KAYFSANNMNV | | | |
| | DTLYSTRKKDNST | | | KVKTINGSFTDY | | | |
| | YIVQTIKDIYAKDN | | | LRKVWKFKKER | | | |
| | TTLKKQFDKSPEKF | | | NHGYKHHAEDA | | | |
| | LMYQHDPRTFEKL | | | LIIANADFLFKE | | | |
| | EVIMKQYANEKNP | | | NKKLKAVNSVL | | | |
| | LAKYHEETGEYLT | | | EKPEIETKQLDIQ | | | |
| | KYSKKNNGPIVKSL | | | VDSEDNYSEMFI | | | |
| | KYIGNKLGSHLDVT | | | IPKQVQDIKDFR | | | |
| | HQFKSSTKKLVKLS | | | NFKYSHRVDKK | | | |
| | IKNYRFDVYLTEKG | | | PNRQLINDTLYS | | | |
| | YKFVTIAYLNVFKK | | | TRKKDNSTYIVQ | | | |
| | DNYYYIPKDKYQE | | | TIKDIYAKDNTT | | | |
| | LKEKKKIKDTDQFI | | | LKKQFDKSPEKF | | | |
| | ASFYKNDLIKLNGD | | | LMYQHDPRTFE | | | |
| | LYKIIGVNSDDRNII | | | KLEVIMKQYAN | | | |
| | ELDYYDIKYKDYC | | | EKNPLAKYHEE | | | |
| | EINNIKGEPRIKKTI | | | TGEYLTKYSKK | | | |
| | GKKTESIEKFTTDV | | | NNGPIVKSLKYI | | | |
| | LGNLYLHSTEKAPQ | | | GNKLGSHLDVT | | | |
| | LIFKRGL | | | HQFKSSTKKLV | | | |
| | | | | KLSIKNYRFDVY | | | |
| | | | | LTEKGYKFVTIA | | | |
| | | | | YLNVFKKDNYY | | | |
| | | | | YIPKDKYQELKE | | | |
| | | | | KKKIKDTDQFIA | | | |
| | | | | SFYKNDLIKLNG | | | |
| | | | | DLYKIIGVNSDD | | | |
| | | | | RNIIELDYYDIK | | | |
| | | | | YKDYCEINNIKG | | | |
| | | | | EPRIKKTIGKKT | | | |
| | | | | ESIEKFTTDVLG | | | |
| | | | | NLYLHSTEKAPQ | | | |
| | | | | LIFKRGL | | | |
| 8686 | NQKFILGLDIGITSV | N585A | 8694 | NQKFILGLDIGIT | sRGN3.3 | NA | NNGG |
| | GYGLIDYETKNIID | | | SVGYGLIDYETK | | | |
| | AGVRLFPEANVEN | | | NIIDAGVRLFPE | | | |
| | NEGRRSKRGSRRL | | | ANVENNEGRRS | | | |
| | KRRRIHRLERVKLL | | | KRGSRRLKRRRI | | | |
| | LTEYDLINKEQIPTS | | | HRLERVKLLLTE | | | |
| | NNPYQIRVKGLSEI | | | YDLINKEQIPTS | | | |
| | LSKDELAIALLHLA | | | NNPYQIRVKGLS | | | |
| | KRRGIHNVDVAAD | | | EILSKDELAIALL | | | |
| | KEETASDSLSTKDQ | | | HLAKRRGIHNV | | | |
| | INKNAKFLESRYVC | | | DVAADKEETAS | | | |
| | ELQKERLENEGHV | | | DSLSTKDQINKN | | | |
| | RGVENRFLTKDIVR | | | AKFLESRYVCEL | | | |
| | EAKKIIDTQMQYYP | | | QKERLENEGHV | | | |
| | EIDETFKEKYISLVE | | | RGVENRFLTKDI | | | |
| | TRREYFEGPGQGSP | | | VREAKKIIDTQM | | | |
| | FGWNGDLKKWYE | | | QYYPEIDETFKE | | | |
| | MLMGHCTYFPQEL | | | KYISLVETRREY | | | |
| | RSVKYAYSADLEN | | | FEGPGQGSPFG | | | |
| | ALNDLNNLIIQRDN | | | WNGDLKKWYE | | | |
| | SEKLEYHEKYHIIE | | | MLMGHCTYFPQ | | | |
| | NVFKQKKKPTLKQI | | | ELRSVKYAYSA | | | |
| | AKEIGVNPEDIKGY | | | DLFNALNDLNN | | | |
| | RITKSGTPEFTSFKL | | | LIIQRDNSEKLE | | | |
| | FHDLKKVVKDHAI | | | YHEKYHIIENVF | | | |
| | LDDIDLLNQIAEILT | | | KQKKKPTLKQIA | | | |
| | IYQDKDSIVAELGQ | | | KEIGVNPEDIKG | | | |
| | LEYLMSEADKQSIS | | | YRITKSGTPEFTS | | | |
| | ELTGYTGTHSLSLK | | | FKLFHDLKKVV | | | |
| | CMNMIIDELWHSS | | | KDHAILDDIDLL | | | |
| | MNQMEVFTYLNM | | | NQIAEILTIYQDK | | | |
| | RPKKYELKGYQRIP | | | DSIVAELGQLEY | | | |
| | TDMIDDAILSPVVK | | | LMSEADKQSISE | | | |
| | RTFIQSINVINKVIE | | | LTGYTGTHSLSL | | | |
| | KYGIPEDIIIELARE | | | KCMNMIIDELW | | | |
| | NNSDDRKKFINNLQ | | | HSSMNQMEVFT | | | |
| | KKNEATRKRINEIIG | | | YLNMRPKKYEL | | | |
| | QTGNQNAKRIVEKI | | | KGYQRIPTDMID | | | |
| | RLHDQQEGKCLYS | | | DAILSPVVKRTFI | | | |
| | LESIPLEDLLNNPN | | | QSINVINKVIEK | | | |
| | HYEVDHIIPRSVSFD | | | YGIPEDIIIELARE | | | |
| | NSYHNKVLVKQSE | | | NNSDDRKKFINN | | | |
| | NSKKSNLTPYQYFN | | | LQKKNEATRKRI | | | |
| | SGKSKLSYNQFKQ | | | NEIIGQTGNQNA | | | |
| | HILNLSKSQDRISK | | | KRIVEKIRLHDQ | | | |
| | KKKEYLLEERDINK | | | QEGKCLYSLESI | | | |
| | FEVQKEFINRNLVD | | | PLEDLLNNPNHY | | | |
| | TRYATRELTSYLKA | | | EVDHIIPRSVSFD | | | |
| | YFSANNMDVKVKT | | | NSYHNKVLVKQ | | | |
| | INGSFTNHLRKVW | | | SEASKKSNLTPY | | | |
| | RFDKYRNHGYKHH | | | QYFNSGKSKLSY | | | |
| | AEDALIIANADFLF | | | NQFKQHILNLSK | | | |
| | KENKKLQNTNKILE | | | SQDRISKKKKEY | | | |
| | KPTIENNTKKVTVE | | | LLEERDINKFEV | | | |
| | KEEDYNNVFETPKL | | | QKEFINRNLVDT | | | |
| | VEDIKQYRDYKFSH | | | RYATRELTSYLK | | | |
| | RVDKKPNRQLINDT | | | AYFSANNMDVK | | | |
| | LYSTRMKDEHDYI | | | VKTINGSFTNHL | | | |
| | VQTITDIYGKDNTN | | | RKVWRFDKYRN | | | |
| | LKKQFNKNPEKFL | | | HGYKHHAEDAL | | | |
| | MYQNDPKTFEKLSI | | | IIANADFLFKEN | | | |
| | IMKQYSDEKNPLA | | | KKLQNTNKILEK | | | |
| | KYYEETGEYLTKY | | | PTIENNTKKVTV | | | |
| | SKKNNGPIVKKIKL | | | EKEEDYNNVFE | | | |
| | LGNKVGNHLDVTN | | | TPKLVEDIKQYR | | | |
| | KYENSTKKLVKLSI | | | DYKFSHRVDKK | | | |
| | KNYRFDVYLTEKG | | | PNRQLINDTLYS | | | |
| | YKFVTIAYLNVFKK | | | TRMKDEHDYIV | | | |
| | DNYYYIPKDKYQE | | | QTITDIYGKDNT | | | |
| | LKEKKKIKDTDQFI | | | NLKKQFNKNPE | | | |
| | ASFYKNDLIKLNGD | | | KFLMYQNDPKT | | | |
| | LYKIIGVNSDDRNII | | | FEKLSIIMKQYS | | | |
| | ELDYYDIKYKDYC | | | DEKNPLAKYYE | | | |
| | EINNIKGEPRIKKTI | | | ETGEYLTKYSK | | | |
| | GKKTESIEKFTTDV | | | KNNGPIVKKIKL | | | |
| | LGNLYLHSTEKAPQ | | | LGNKVGNHLDV | | | |
| | LIFKRGL | | | TNKYENSTKKL | | | |
| | | | | VKLSIKNYRFDV | | | |
| | | | | YLTEKGYKFVTI | | | |
| | | | | AYLNVFKKDNY | | | |
| | | | | YYIPKDKYQELK | | | |
| | | | | EKKKIKDTDQFI | | | |
| | | | | ASFYKNDLIKLN | | | |
| | | | | GDLYKIIGVNSD | | | |
| | | | | DRNIIELDYYDI | | | |
| | | | | KYKDYCEINNIK | | | |
| | | | | GEPRIKKTIGKK | | | |
| | | | | TESIEKFTTDVL | | | |
| | | | | GNLYLHSTEKAP | | | |
| | | | | QLIFKRGL | | | |
| 8687 | DKKYSIGLDIGTNS | H840A | 8695 | DKKYSIGLDIGT | SpG | NA | NGN |
| | VGWAVITDEYKVP | | | NSVGWAVITDE | | | |
| | SKKFKVLGNTDRH | | | YKVPSKKFKVL | | | |
| | SIKKNLIGALLFDSG | | | GNTDRHSIKKNL | | | |
| | ETAEATRLKRTARR | | | IGALLFDSGETA | | | |
| | RYTRRKNRICYLQE | | | EATRLKRTARR | | | |
| | IFSNEMAKVDDSFF | | | RYTRRKNRICYL | | | |
| | HRLEESFLVEEDKK | | | QEIFSNEMAKVD | | | |
| | HERHPIFGNIVDEV | | | DSFFHRLEESFL | | | |
| | AYHEKYPTIYHLRK | | | VEEDKKHERHPI | | | |
| | KLVDSTDKADLRLI | | | FGNIVDEVAYHE | | | |
| | YLALAHMIKFRGH | | | KYPTIYHLRKKL | | | |
| | FLIEGDLNPDNSDV | | | VDSTDKADLRLI | | | |
| | DKLFIQLVQTYNQL | | | YLALAHMIKFR | | | |
| | FEENPINASGVDAK | | | GHFLIEGDLNPD | | | |
| | AILSARLSKSRRLE | | | NSDVDKLFIQLV | | | |
| | NLIAQLPGEKKNGL | | | QTYNQLFEENPI | | | |
| | FGNLIALSLGLTPNF | | | NASGVDAKAILS | | | |
| | KSNFDLAEDAKLQ | | | ARLSKSRRLENL | | | |
| | LSKDTYDDDLDNL | | | IAQLPGEKKNGL | | | |
| | LAQIGDQYADLFLA | | | FGNLIALSLGLT | | | |
| | AKNLSDAILLSDILR | | | PNFKSNFDLAED | | | |
| | VNTEITKAPLSASM | | | AKLQLSKDTYD | | | |
| | IKRYDEHHQDLTLL | | | DDLDNLLAQIG | | | |
| | KALVRQQLPEKYK | | | DQYADLFLAAK | | | |
| | EIFFDQSKNGYAGY | | | NLSDAILLSDILR | | | |
| | IDGGASQEEFYKFI | | | VNTEITKAPLSA | | | |
| | KPILEKMDGTEELL | | | SMIKRYDEHHQ | | | |
| | VKLNREDLLRKQR | | | DLTLLKALVRQ | | | |
| | TFDNGSIPHQIHLGE | | | QLPEKYKEIFFD | | | |
| | LHAILRRQEDFYPF | | | QSKNGYAGYID | | | |
| | LKDNREKIEKILTFR | | | GGASQEEFYKFI | | | |
| | IPYYVGPLARGNSR | | | KPILEKMDGTEE | | | |
| | FAWMTRKSEETITP | | | LLVKLNREDLLR | | | |
| | WNFEEVVDKGASA | | | KQRTFDNGSIPH | | | |
| | QSFIERMTNFDKNL | | | QIHLGELHAILR | | | |
| | PNEKVLPKHSLLYE | | | RQEDFYPFLKDN | | | |
| | YFTVYNELTKVKY | | | REKIEKILTFRIP | | | |
| | VTEGMRKPAFLSG | | | YYVGPLARGNS | | | |
| | EQKKAIVDLLFKTN | | | RFAWMTRKSEE | | | |
| | RKVTVKQLKEDYF | | | TITPWNFEEVVD | | | |
| | KKIECFDSVEISGVE | | | KGASAQSFIERM | | | |
| | DRFNASLGTYHDL | | | TNFDKNLPNEK | | | |
| | LKIIKDKDFLDNEE | | | VLPKHSLLYEYF | | | |
| | NEDILEDIVLTLTLF | | | TVYNELTKVKY | | | |
| | EDREMIEERLKTYA | | | VTEGMRKPAFL | | | |
| | HLFDDKVMKQLKR | | | SGEQKKAIVDLL | | | |
| | RRYTGWGRLSRKLI | | | FKTNRKVTVKQ | | | |
| | NGIRDKQSGKTILD | | | LKEDYFKKIECF | | | |
| | FLKSDGFANRNFM | | | DSVEISGVEDRF | | | |
| | QLIHDDSLTFKEDI | | | NASLGTYHDLL | | | |
| | QKAQVSGQGDSLH | | | KIIKDKDFLDNE | | | |
| | EHIANLAGSPAIKK | | | ENEDILEDIVLTL | | | |
| | GILQTVKVVDELV | | | TLFEDREMIEER | | | |
| | KVMGRHKPENIVIE | | | LKTYAHLFDDK | | | |
| | MARENQTTQKGQK | | | VMKQLKRRRYT | | | |
| | NSRERMKRIEEGIK | | | GWGRLSRKLIN | | | |
| | ELGSQILKEHPVEN | | | GIRDKQSGKTIL | | | |
| | TQLQNEKLYLYYL | | | DFLKSDGFANR | | | |
| | QNGRDMYVDQEL | | | NFMQLIHDDSLT | | | |
| | DINRLSDYDVDHIV | | | FKEDIQKAQVSG | | | |
| | PQSFLKDDSIDNKV | | | QGDSLHEHIANL | | | |
| | LTRSDKNRGKSDN | | | AGSPAIKKGILQ | | | |
| | VPSEEVVKKMKNY | | | TVKVVDELVKV | | | |
| | WRQLLNAKLITQR | | | MGRHKPENIVIE | | | |
| | KFDNLTKAERGGL | | | MARENQTTQKG | | | |
| | SELDKAGFIKRQLV | | | QKNSRERMKRIE | | | |
| | ETRQITKHVAQILD | | | EGIKELGSQILKE | | | |
| | SRMNTKYDENDKL | | | HPVENTQLQNE | | | |
| | IREVKVITLKSKLVS | | | KLYLYYLQNGR | | | |
| | DFRKDFQFYKVREI | | | DMYVDQELDIN | | | |
| | NNYHHAHDAYLN | | | RLSDYDVDAIVP | | | |
| | AVVGTALIKKYPKL | | | QSFLKDDSIDNK | | | |
| | ESEFVYGDYKVYD | | | VLTRSDKNRGK | | | |
| | VRKMIAKSEQEIGK | | | SDNVPSEEVVK | | | |
| | ATAKYFFYSNIMNF | | | KMKNYWRQLL | | | |
| | FKTEITLANGEIRKR | | | NAKLITQRKFDN | | | |
| | PLIETNGETGEIVW | | | LTKAERGGLSEL | | | |
| | DKGRDFATVRKVL | | | DKAGFIKRQLVE | | | |
| | SMPQVNIVKKTEV | | | TRQITKHVAQIL | | | |
| | QTGGFSKESILPKR | | | DSRMNTKYDEN | | | |
| | NSDKLIARKKDWD | | | DKLIREVKVITL | | | |
| | PKKYGGFLWPTVA | | | KSKLVSDFRKDF | | | |
| | YSVLVVAKVEKGK | | | QFYKVREINNY | | | |
| | SKKLKSVKELLGITI | | | HHAHDAYLNAV | | | |
| | MERSSFEKNPIDFL | | | VGTALIKKYPKL | | | |
| | EAKGYKEVKKDLII | | | ESEFVYGDYKV | | | |
| | KLPKYSLFELENGR | | | YDVRKMIAKSE | | | |
| | KRMLASAKQLQKG | | | QEIGKATAKYFF | | | |
| | NELALPSKYVNFLY | | | YSNIMNFFKTEI | | | |
| | LASHYEKLKGSPED | | | TLANGEIRKRPLI | | | |
| | NEQKQLFVEQHKH | | | ETNGETGEIVW | | | |
| | YLDEIIEQISEFSKR | | | DKGRDFATVRK | | | |
| | VILADANLDKVLSA | | | VLSMPQVNIVK | | | |
| | YNKHRDKPIREQAE | | | KTEVQTGGFSKE | | | |
| | NIIHLFTLTNLGAPA | | | SILPKRNSDKLIA | | | |
| | AFKYFDTTIDRKQY | | | RKKDWDPKKY | | | |
| | RSTKEVLDATLIHQ | | | GGFLWPTVAYS | | | |
| | SITGLYETRIDLSQL | | | VLVVAKVEKGK | | | |
| | GGD | | | SKKLKSVKELLG | | | |
| | | | | ITIMERSSFEKNP | | | |
| | | | | IDFLEAKGYKEV | | | |
| | | | | KKDLIIKLPKYS | | | |
| | | | | LFELENGRKRM | | | |
| | | | | LASAKQLQKGN | | | |
| | | | | ELALPSKYVNFL | | | |
| | | | | YLASHYEKLKG | | | |
| | | | | SPEDNEQKQLFV | | | |
| | | | | EQHKHYLDEIIE | | | |
| | | | | QISEFSKRVILAD | | | |
| | | | | ANLDKVLSAYN | | | |
| | | | | KHRDKPIREQAE | | | |
| | | | | NIIHLFTLTNLGA | | | |
| | | | | PAAFKYFDTTID | | | |
| | | | | RKQYRSTKEVL | | | |
| | | | | DATLIHQSITGL | | | |
| | | | | YETRIDLSQLGG | | | |
| | | | | D | | | |

### Flap Endonuclease

In some embodiments, a split prime editor further comprises additional polypeptide components, for example, a flap endonuclease (FEN, e.g., FEN1). In some embodiments, the flap endonuclease excises the 5′ single stranded DNA of the edit strand of the target gene and assists incorporation of the intended nucleotide edit into the target gene. In some embodiments, the FEN is linked or fused to another component. In some embodiments, the FEN is provided in trans, for example, as a separate polypeptide or polynucleotide encoding the FEN. In some embodiments, a split prime editor or prime editing composition comprises a flap nuclease. In some embodiments, the flap nuclease is a FEN1, or any FEN1 functional variant, functional mutant, or functional fragment thereof. In some embodiments, the flap nuclease is a TREX2, EXO1, or any other flap nuclease known in the art, or any functional variant, functional mutant, or functional fragment thereof. In some embodiments, the flap nuclease has amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the flap nucleases described herein or known in the art.

### Nuclear Localization Sequences

In some embodiments, a split prime editor further comprises one or more nuclear localization sequence (NLS). In some embodiments, the NLS helps promote translocation of a protein into the cell nucleus. In some embodiments, a split prime editor comprises a DNA binding domain and a DNA polymerase that comprises one or more NLSs. In some embodiments, the split prime editor comprises a first polypeptide comprising a first amino acid sequence and a second polypeptide comprising a second amino acid sequence. In some embodiments, one or more polypeptides of the split prime editor are fused to or linked to one or more NLSs. In some embodiments, the split prime editor comprises a first amino acid sequence and a second amino acid sequence that are provided in trans, wherein the first amino acid sequence and/or the second amino acid sequence is fused or linked to one or more NLSs.

In some embodiments, the first polypeptide comprises at least one NLS. In some embodiments, the second polypeptide comprises at least one NLS. In some embodiments, the at least one NLS comprises an amino acid sequence as set forth in Table 3.

In certain embodiments, a split prime editor or prime editing complex comprises at least one NLS. In some embodiments, a split prime editor or prime editing complex comprises at least two NLSs. In embodiments with at least two NLSs, the NLSs can be the same NLS, or they can be different NLSs.

In some instances, a split prime editor may further comprise at least one nuclear localization sequence (NLS). In some cases, a split prime editor may further comprise 1 NLS. In some cases, a split prime editor may further comprise 2 NLSs. In other cases, a split prime editor may further comprise 3 NLSs. In one case, a primer editor may further comprise more than 4, 5, 6, 7, 8, 9 or 10 NLSs.

In addition, the NLSs may be expressed as part of a split prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within the sequence(s) of a split prime editor or a component thereof (e.g., inserted between the DNA-binding domain and the DNA polymerase domain of a split prime editor, between the DNA binding domain and a linker sequence, between a DNA polymerase and a linker sequence, between two linker sequences of a split prime editor or a component thereof, in either N-terminus to C-terminus or C-terminus to N-terminus order). In some embodiments, a split prime editor is a protein that comprises an NLS at the N terminus. In some embodiments, a split prime editor is a protein that comprises an NLS at the C terminus. In some embodiments, a split prime editor is a protein that comprises at least one NLS at both the N terminus and the C terminus. In some embodiments, the split prime editor is a protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a split prime editor comprise bipartite NLSs. In some embodiments, a nuclear localization signal (NLS) is predominantly basic. In some embodiments, the one or more NLSs of a split prime editor are rich in lysine and arginine residues. In some embodiments, the one or more NLSs of a split prime editor comprise proline residues. In some embodiments, a nuclear localization signal (NLS) comprises the sequence MDSLLMNRRKFLYQFKNVRWAKGRRETYLC (SEQ ID NO: 8696), KRTADGSEFESPKKKRKV (SEQ ID NO: 8697), KRTADGSEFEPKKKRKV (SEQ ID NO: 8698), NLSKRPAAIKKAGQAKKKK (SEQ ID NO: 8699), RQRRNELKRSF (SEQ ID NO: 8700), or NQSSNFGPMKGGNFGGRSSGPYGGGGQYFAKPRNQGGY (SEQ ID NO: 8701).

In some embodiments, a NLS is a monopartite NLS. For example, in some embodiments, a NLS is a SV40 large T antigen NLS PKKKRKV (SEQ ID NO: 8702). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer amino acid sequence comprises the Xenopus nucleoplasmin sequence KRXXXXXXXXXXKKKL (SEQ ID NO: 4451) wherein X is any amino acid. In some embodiments, a NLS is a noncanonical sequences such as M9 of the hnRNP A1 protein, the influenza virus nucleoprotein NLS, and the yeast Gal4 protein NLS.

Other non-limiting examples of NLS sequences are provided in Table 3 below. In some embodiments, the first polypeptide comprises a NLS sequence (e.g., and NLS sequence disclosed in Table 3). In some embodiments, the second polypeptide comprises a NLS sequence (e.g., and NLS sequence disclosed in Table 3). The NLS sequence may comprise any one of the sequences disclosed in table 3.

TABLE 3
Exemplary nuclear localization sequences

| | SEQ | |
|---|---|---|
| | ID | |
| Description | NO: | Sequence |
| NLS of SV40 | 8702 | PKKKRKV |
| Large T-AG | | |
| NLS | 8703 | MKRTADGSEFESPKKKRKV |
| NLS | 8697 | KRTADGSEFESPKKKRKV |
| NLS | 8696 | MDSLLMNRRKFLYQFKNVRWAKGRR |
| | | ETYLC |
| NLS of | 8704 | AVKRPAATKKAGQAKKKKLD |
| Nucleoplasmin | | |
| NLS of EGL-13 | 8705 | MSRRRKANPTKLSENAKKLAKEVEN |
| NLS of C-Myc | 8706 | PAAKRVKLD |
| NLS of Tus- | 8707 | KLKIKRPVK |
| protein | | |
| NLS of polyoma | 8708 | VSRKRPRP |
| large T-AG | | |
| NLS of Hepatitis | 8709 | EGAPPAKRAR |
| D virus antigen | | |
| NLS of murine p53 | 8710 | PPQPKKKPLDGE |
| Linker + NLS | 8711 | SGGSKRTADGSEFEPKKKRKV |

### Additional Split Prime Editor Components

A split prime editor described herein may comprise additional functional domains, for example, one or more domains that modify the folding, solubility, or charge of the split prime editor. In some instances, the split prime editor may comprise a solubility-enhancement (SET) domain.

In some embodiments, a split prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligasc tags, FlAsH tags, V5 tags, and SBP-tags. Additional suitable sequences will be apparent to those of skill in the art. In some embodiments, the fusion protein comprises one or more His tags.

In some embodiments, a split prime editor comprises one or more polypeptide domains encoded by one or more reporter genes. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a split prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

In some embodiments, a split prime editor comprises a protein domain that is capable of modifying the intracellular half-life of the split prime editor.

In some embodiments, a prime editing complex comprises at least two polypeptides comprising a DNA binding domain (e.g., Cas9 (H840A)) and a reverse transcriptase (e.g., a variant MMLV RT) having the following structure: [NLS]-[Cas9 (H840A)]-[linker]-[MMLV_RT (D200N) (T330P) (L603W) (T306K) (W313F)], and a desired PEgRNA.

Polypeptides comprising components of a split prime editor may be fused via peptide linkers, or may be provided in trans relevant to each other. For example, a reverse transcriptase may be expressed, delivered, or otherwise provided as an individual component rather than as a part of a protein with the DNA binding domain. In such cases, components of the split prime editor may be associated through non-peptide linkages or co-localization functions. In some embodiments, a split prime editor further comprises additional components capable of interacting with, associating with, or capable of recruiting other components of the split prime editor or the prime editing system. For example, a split prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the split prime editor comprises a DNA binding domain fused or linked to an RNA-protein recruitment polypeptide. In some embodiments, the split prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PERNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In some embodiments, a split prime editor comprises a polypeptide domain, an MS2 coat protein (MCP), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 4446). In some embodiments, the amino acid sequence of the MCP is:

(SEQ ID NO: 4447)
GSASNFTQFVLVDNGGTGDVTVAPSNFANGVAEWISSNSRSQAYKVTC
SVRQSSAQNRKYTIKVEVPKVATQTVGGEELPVAGWRSYLNMELTIPI
FATNSDCELIVKAMQGLLKDGNPIPSAIA ANSGIY.

In certain embodiments, components of a split prime editor are directly fused to each other. In certain embodiments, components of a split prime editor are associated to each other via a linker.

As used herein, a linker can be any chemical group or a molecule linking two molecules or moieties, e.g., a DNA binding domain and a polymerase domain of a split prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In some embodiments, the second polypeptide further comprises at least one (e.g., 1, 2, 3, 4, 5, 6, 7, 8, 9, or 10) peptide linker(s). In some embodiments, the first polypeptide further comprises at least one (e.g., 1, 2, 3, 4, 5, 6, 7, 8, 9, or 10) peptide linker(s).

In certain embodiments, two or more components of a split prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the at least one peptide linker comprises 1 to 100 amino acids, for example, the peptide linker may be from 5 to 25 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 8712), (G)n (SEQ ID NO: 8713), (EAAAK)n (SEQ ID NO: 8714), (GGS)n (SEQ ID NO: 8715), (SGGS)n (SEQ ID NO: 8716), (XP)n (SEQ ID NO: 8717), or any combination thereof, wherein n is independently an integer between 1 and 30, and wherein X is any amino acid. In some embodiments, the linker comprises the amino acid sequence (GGS)n (SEQ ID NO: 8718), wherein n is 1, 3, or 7. In some embodiments, the linker comprises the amino acid sequence SGSETPGTSESATPES (SEQ ID NO: 8719). In some embodiments, the linker comprises the amino acid sequence SGGSSGGSSGS ETPGTSESATPESSGGSSGGS (SEQ ID NO: 8720). In some embodiments, the linker comprises the amino acid sequence SGGSGGSGGS (SEQ ID NO: 8721). In some embodiments, the linker comprises the amino acid sequence SGGS (SEQ ID NO: 8722). In other embodiments, the linker comprises the amino acid sequence SGGSSGGSSGSETPGTSESATPESAGSYPYDVPDYAGSAAPAAKKKKLDGSGSGGSSGG S (SEQ ID NO: 8723).

In some embodiments, a linker comprises 1-100 amino acids. In some embodiments, the linker comprises the amino acid sequence SGSETPGTSESATPES (SEQ ID NO: 8719). In some embodiments, the linker comprises the amino acid sequence SGGSSGGSSGSETPGTSESATPESSGGSSGGS (SEQ ID NO: 8720). In some embodiments, the linker comprises the amino acid sequence SGGSGGSGGS (SEQ ID NO: 8721). In some embodiments, the linker comprises the amino acid sequence SGGS (SEQ ID NO: 8722). In some embodiments, the linker comprises the amino acid sequence GGSGGS (SEQ ID NO: 8724), (GGSGGSGGS (SEQ ID NO: 8725), SGGSSGGSSGSETPGTSESATPESAGSYPYDVPDYAGSAAPAAKKKKLDGSGSGGSSGGS (SEQ ID NO: 8723), or SGGSSGGSSGSETPGTSESATPESSGGSSGGSS (SEQ ID NO: 8726).

In some embodiments, the at least one peptide linker comprises an amino acid sequence as set forth in Table 15. In some embodiments, the peptide linker may have a secondary structure motif including, but not limited to, a residue isolated B-bridge (referred to as “B” in Table 15), an extended strand (referred to as “E” in Table 15), a 3-helix (referred to as “G” in Table 15), an alpha helix (referred to as “H” in Table 15), a 5-helix (referred to as “I” in Table 15), a hydrogen bonded turn (referred to as “T” in Table 15), a bend (referred to as “S” in Table 15), and/or a coil (referred to as “C” in Table 15). The term “NA” as used in Table 15 refers to “not analyzed.”

TABLE 15
Exemplary peptide linker sequences

| SEQ | | | | | |
|---|---|---|---|---|---|
| ID | | | | | Secondary |
| NO: | Sequence | Length | Name | Type | Structure |
| 8727 | AEAAKEAAKEAAKEAAKALE | 38 | ALEA | Structured | NA |
| | AEAAKEAAKEAAKEAAKA | | | | |
| 8728 | AEAAKEAAKEAAKEAAKALE | 76 | ALEA2 | Structured | NA |
| | AEAAKEAAKEAAKEAAKAAE | | | | |
| | AAKEAAKEAAKEAAKALEAE | | | | |
| | AAKEAAKEAAKEAAKA | | | | |
| 8729 | AGGSQYKLILNGKTLKGETTT | 63 | Basic_ | Structured | NA |
| | EAVNAATAEKVFKQYANRNG | | GB1 | | |
| | VDGKWTYDDATKTFTVTEGG | | | | |
| | SA | | | | |
| 8730 | KLSGGGGSGGGGSGGGGSAE | 141 | cTPR3 | Structured | NA |
| | AWYNLGNAYYKQGDYQKAIE | | | | |
| | YYQKALELDPNNAEAWYNLG | | | | |
| | NAYYKQGDYQKAIEYYQKAL | | | | |
| | ELDPNNAEAWYNLGNAYYKQ | | | | |
| | GDYQKAIEDYQKALELDPNNL | | | | |
| | QRSAGGGGSGGGGSGGGGAS | | | | |
| 8731 | AGGSQYKLILNGKTLKGETTT | 63 | GB1 | Structured | NA |
| | EAVDAATAEKVFKQYANDNG | | | | |
| | VDGEWTYDDATKTFTVTEGG | | | | |
| | SA | | | | |
| 8732 | AGSGNSSGSGGSGGSGNSSGS | 46 | GcGcP | Unstructured | NA |
| | GGSPVPSTPPTPSPSTPPTPSPS | | | | |
| | AS | | | | |
| 8733 | AGGSSGGSSGGSSGGSSGGSS | 47 | GGSS11 | Unstructured | NA |
| | GGSSGGSSGGSSGGSSGGSSG | | | | |
| | GSSAS | | | | |
| 8734 | AGGSSGGSSGGSSGGSSGGSS | 39 | GGSS9 | Unstructured | NA |
| | GGSSGGSSGGSSGGSSAS | | | | |
| 8735 | AGSGGSGGSGGSPVPSTPPTPS | 144 | GPbG | Unstructured | NA |
| | PSTPPTPSPSIQRTPKIQVYSRH | | | | |
| | PAENGKSNFLNCYVSGFHPSDI | | | | |
| | EVDLLKNGERIEKVEHSDLSFS | | | | |
| | KDWSFYLLYYTEFTPTEKDEY | | | | |
| | ACRVNHVTLSQPKIVKWDRD | | | | |
| | GGSGGSGGSGGSAS | | | | |
| 8736 | AGSGGSGGSGGSPVPSTPPTPS | 152 | GPbP | Unstructured | NA |
| | PSTPPTPSPSIQRTPKIQVYSRH | | | | |
| | PAENGKSNFLNCYVSGFHPSDI | | | | |
| | EVDLLKNGERIEKVEHSDLSFS | | | | |
| | KDWSFYLLYYTEFTPTEKDEY | | | | |
| | ACRVNHVTLSQPKIVKWDRDP | | | | |
| | VPSTPPTPSPSTPPTPSPSAS | | | | |
| 8737 | AGSGGSGGSGGSPVPSTPPTNS | 74 | GpCpCpC | Unstructured | NA |
| | SSTPPTPSPSPVPSTPPTNSSSTP | | | | |
| | PTPSPSPVPSTPPTNSSSTPPTPS | | | | |
| | PSAS | | | | |
| 8738 | AGSGGSGGSGGSPVPSTPPTPS | 66 | GPGcP | Unstructured | NA |
| | PSTPPTPSPSGGSGNSSGSGGSP | | | | |
| | VPSTPPTPSPSTPPTPSPSAS | | | | |
| 8739 | AGSGGSGGSGGSPVPSTPPTPS | 74 | GPPcP | Unstructured | NA |
| | PSTPPTPSPSPVPSTPPTNSSSTP | | | | |
| | PTPSPSPVPSTPPTPSPSTPPTPS | | | | |
| | PSAS | | | | |
| 8740 | AGSGGSGGSGGSPVPSTPPTPS | 74 | GPPP | Unstructured | NA |
| | PSTPPTPSPSPVPSTPPTPSPSTP | | | | |
| | PTPSPSPVPSTPPTPSPSTPPTPS | | | | |
| | PSAS | | | | |
| 8741 | AGSGGSGGSGGSPVPSTPPTPS | 121 | GPUG | Unstructured | NA |
| | PSTPPTPSPSQIFVKTLTGKTITL | | | | |
| | EVEPSDTIENVKAKIQDKEGIP | | | | |
| | PDQQRLIFAGKQLEDGRTLSD | | | | |
| | YNIQKESTLHLVLRLRGGGGS | | | | |
| | GGSGGSGGSAS | | | | |
| 8742 | AGGGSGGGGSGGGGSGGGGS | 52 | GS11 | Unstructured | NA |
| | GGGGSGGGGSGGGGSGGGGS | | | | |
| | GGGGSGGGGSAS | | | | |
| 8743 | AGGGGSGGGGSGGGGSGGGG | 33 | GS6 | Unstructured | NA |
| | SGGGGSGGGGSAS | | | | |
| 8744 | AGGGSGGGGSGGGGSGGGGS | 37 | GS7 | Unstructured | NA |
| | GGGGSGGGGSGGGGSAS | | | | |
| 8745 | AGGGSGGGGSGGGGSGGGGS | 42 | GS8 | Unstructured | NA |
| | GGGGSGGGGSGGGGSGGGGS | | | | |
| | AS | | | | |
| 8746 | AGGGSGGGGSGGGGSGGGGS | 47 | GS9 | Unstructured | NA |
| | GGGGSGGGGSGGGGSGGGGS | | | | |
| | GGGGSAS | | | | |
| 8747 | AGGSSGGSSGGSSGGSSGGSS | 31 | GSS7 | Unstructured | NA |
| | GGSSGGSSAS | | | | |
| 8748 | AGSQMALHANVTGAMNYTW | 36 | Nat1 | Natural | CCHHHHHH |
| | ATCTINTHAPRSMLGSA | | | | HHHHHSSSS |
| | | | | | SSSTTCTTTT |
| | | | | | TSTTTSSSS |
| 8749 | GRMANFDGMDMSHKMALSST | 43 | Nat10 | Natural | SSTTCSSSCC |
| | NEIETNEGLAGTSLDVMDLSR | | | | CCCCCSSCT |
| | VL | | | | TCCCCCCTT |
| | | | | | TTSCSSCTTS |
| | | | | | HHHHH |
| 8750 | SAAAATPAVRTVPQYKYAAG | 49 | Nat11 | Natural | CCSCCSSCC |
| | VRNPQQHLNAQPQVTMQQPA | | | | CSCSSCCSSC |
| | VHVQGQEPL | | | | CCSSTTTTC |
| | | | | | CCCSCCCCC |
| | | | | | CCCSCTTCC |
| | | | | | CCC |
| 8751 | VSGITGMVDPSRINVANLAEE | 50 | Nat12 | Natural | EEEEESCCC |
| | GLGNIRANSFGYDSAAIKLRIH | | | | GGGEEEEEE |
| | KLSKTLD | | | | CCTTCCSCC |
| | | | | | CCCCSSSEE |
| | | | | | EEEECCTTT |
| | | | | | CSSSC |
| 8752 | ILTHDSSIRYLQEIYNSNNQKIV | 51 | Nat13 | Natural | TTTGGGGTS |
| | NLKEKVAQLEAQCQEPCKDT | | | | GGGHHHHH |
| | VQIHDITG | | | | HHHHHHHH |
| | | | | | HHHHHHHH |
| | | | | | HHSCSCCCC |
| | | | | | SCCCEEEEE |
| 8753 | KRSVKNPYPISFLLSDLINRRT | 57 | Nat14 | Natural | EEEECCSSCS |
| | QRVDGQPMIGMSSQVEEVRV | | | | CSSSCCCSSC |
| | YEDTEELPGDPDMIR | | | | CCCCCCSSC |
| | | | | | CSGGGCCCC |
| | | | | | CCCCCCSCC |
| | | | | | SCCSCTTCE |
| | | | | | E |
| 8754 | CYGKKYGPKGKGKGMGAGTL | 58 | Nat15 | Natural | HHHHHHCC |
| | STDKGESLGIKYEEGQSHRPTN | | | | CCCCCCCCC |
| | PNASRMAQKVGGSDGC | | | | CCCCCCCCC |
| | | | | | CCCCCCCCC |
| | | | | | CCCCCCCCC |
| | | | | | CCCCCCCCC |
| | | | | | CCEEC |
| 8755 | VPSERGLQRRRFVQNALNG | 19 | Nat16 | Natural | CCCTTTTCS |
| | | | | | SSSSSCCCCT |
| 8756 | LLAPTRIYVKSVLEL | 15 | Nat17 | Natural | SSHHSSSSSS |
| | | | | | SSHHH |
| 8757 | VSPVASFNTLQLGERGNIV | 19 | Nat18 | Natural | SSSCCCSSSC |
| | | | | | CCCTTTTSS |
| 8758 | TEEPGAPLTTPPTLHGNQARA | 21 | Nat19 | Natural | TSCTTSCSC |
| | | | | | CCCCCCTTS |
| | | | | | CCH |
| 8759 | GYETIPLALPAFFPAPDNRGVE | 34 | Nat2 | Natural | CGGGSCCCC |
| | APYRKEQRLGSA | | | | CCCCCCGG |
| | | | | | GTTTTHHHH |
| | | | | | HHHHH |
| 8760 | DVHNFSIKDVGTIITNKTGVSP | 22 | Nat20 | Natural | HHHHHHCC |
| | | | | | CTTCEEEESS |
| | | | | | CCCS |
| 8761 | GECLKCIYNTAGFYCDRCKEG | 21 | Nat21 | Natural | CCBCCBCTT |
| | | | | | EETTTTCEE |
| | | | | | CTT |
| 8762 | QMALHANVTGAMNYTWATC | 30 | Nat22 | Natural | HHHHTCSTT |
| | TINTHAPRSML | | | | SCCCSCCCS |
| | | | | | SHHHHSCCS |
| | | | | | CCH |
| 8763 | PPEATQNVAESTHNLTRNFPA | 25 | Nat23 | Natural | CCCCBCSSS |
| | DLFN | | | | CBCCCCCEE |
| | | | | | CCCCCCC |
| 8764 | AGSVAETLKDNTQSKLTVKGN | 35 | Nat3 | Natural | CCHHHHHH |
| | LDTYGFCDDVWTFI | | | | HHHHHSSSS |
| | | | | | SSTTCTTTTT |
| | | | | | STTTSSSS |
| 8765 | YVREEVFTNNADVVAEKALK | 32 | Nat4 | Natural | ECCCCEECC |
| | PESDITFSKQTA | | | | CCCCCCTTS |
| | | | | | CCCCCCEEC |
| | | | | | CCEEE |
| 8766 | TCHHRSPLSLTPPKCGSCHTKE | 33 | Nat5 | Natural | GTSCSSCCC |
| | IDAADPGRPNL | | | | SSCCCHHHH |
| | | | | | SCSSCCTTST |
| | | | | | TSCCH |
| 8767 | LDTTAENQAKNEHLQKENERL | 34 | Nat6 | Natural | CHHHHHHH |
| | LRDWNDVQGRFEK | | | | HHHHHHHH |
| | | | | | HHHHHHHH |
| | | | | | HHTHHHHH |
| | | | | | HC |
| 8768 | AQAERQRILERTNEGRQEAMA | 34 | Nat7 | Natural | HHHHHHHH |
| | KGVVFGRKRKIDR | | | | HHHHHHHH |
| | | | | | HHHHHHTC |
| | | | | | CCSSCCCSC |
| | | | | | H |
| 8769 | GVTPSTTALPDIVNLSTNYLDK | 35 | Nat8 | Natural | SCCCCCCSS |
| | NTREDRIHSIKDF | | | | SCCCCCCHH |
| | | | | | HHTTTTCCS |
| | | | | | SCCCHHHH |
| 8770 | TKLPEAQQRVGGCFLNLMPQ | 39 | Nat9 | Natural | HTTTCTTCC |
| | MKTLYLTYCANHPSAVNVL | | | | HHHHHHHH |
| | | | | | HHHHHHHH |
| | | | | | HHHHHHHH |
| | | | | | HHHHHH |
| 8726 | SGGSSGGSSGSETPGTSESATP | 33 | PE2 | Unstructured | NA |
| | ESSGGSSGGSS | | | | |
| 8771 | GGSWCIFVYNLSPDSDESVLW | 85 | RNP_1 | Structured | NA |
| | QLFGPFGAVNNVKVIRDENTN | | | | |
| | KCKGFGFVTMTNYDEAAMAI | | | | |
| | ASLNGYRLGDRVLQVSFKTNG | | | | |
| | GS | | | | |
| 8772 | AGSKPFGKSKGFGFVCFSSPDE | 62 | RNP_2 | Structured | NA |
| | ASKAVTEMNQRMVNGKPLYV | | | | |
| | ALAQRKDVRRSQLEASIGSA | | | | |
| 8773 | SGGSSGGSSGS | 11 | | Unstructured | NA |
| 8722 | SGGS | 4 | | Unstructured | NA |
| 8718 | GGS | 3 | | Unstructured | NA |

In certain embodiments, two or more components of a split prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a split prime editor may be able to join or connect to each other in any order.

In some embodiments, a split prime editor protein, a polypeptide component of a split prime editor, or a polynucleotide encoding the split prime editor protein or polypeptide component, may be split into an N-terminal half and a C-terminal half or polypeptides that encode the N-terminal half and the C terminal half, and provided to a target DNA in a cell separately. For example, in certain embodiments, a split prime editor protein may be split into a N-terminal and a C-terminal half for separate delivery in AAV vectors, and subsequently translated and colocalized in a target cell to reform the complete polypeptide or split prime editor protein. In such cases, separate halves of a protein may each comprise a split-intein to facilitate colocalization and reformation of the complete protein by the mechanism of intein facilitated trans splicing. In some embodiments, a split prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete split prime editor protein in the target cell.

In some embodiments, a split prime editor comprises a Cas9 (H840A)nickase and a wild type M-MLV RT (referred to as “PE1”, and a prime editing system or composition referred to as PE1 system or PE1 composition). In some embodiments, a split prime editor comprises one or more individual components of PE1. In some embodiments, a split prime editor protein comprises a Cas9 (H840A)nickase and a M-MLV RT that has amino acid substitutions D200N, T330P, T306K, W313F, and L603W compared to a wild type M-MLV RT (the protein referred to as “PE2”, and a prime editing system or composition referred to as PE2 system or PE2 composition). In some embodiments, a split prime editor protein is PE2. In some embodiments, a split prime editor protein comprises one or more individual components of PE2.

In various embodiments, a split prime editor proteins comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PE1, PE2, or any of the split prime editor sequences described herein or known in the art.

### Scaffold RNA

In certain aspects, the prime editor systems described herein comprise scaffold RNA. The term “scaffold RNA” or “prime editing guide RNA”, or “PEgRNA”, refers to a guide polynucleotide that comprises one or more intended nucleotide edits for incorporation into the target DNA. Such terms can be used interchangeably.

In some embodiments, the first polypeptide and/or the second polypeptide comprises an adapter protein that has affinity for the scaffold RNA. Exemplary adapter proteins include but are not limited to a MS2 coat/adapter protein (MCP), a PP7 adapter protein, a Qβ adapter protein, a F2 adapter protein, a GA adapter protein, a fr adapter protein, a JP501 adapter protein, a M12 adapter protein, a R17 adapter protein, a BZ13 adapter protein, a JP34 adapter protein, a JP500 adapter protein, a KU1 adapter protein, a M11 adapter protein, a MX1 adapter protein, a TW18 adapter protein, a VK adapter protein, a SP adapter protein, a FI adapter protein, a ID2 adapter protein, a NL95 adapter protein, a TW19 adapter protein, a AP205 adapter protein, a ϕCb5 adapter protein, a ϕCb8r adapter protein, a § 12r adapter protein, a ϕCb23r adapter protein, a 7s adapter protein and a PRR1 adapter protein.

In various embodiments, two separate protein domains (e.g., a Cas9 domain and a polymerase domain) may be colocalized to one another to form a functional complex (akin to the function of a protein comprising the two separate protein domains) by using an “RNA-protein recruitment system,” such as the “MS2 tagging technique.” Such systems generally tag one protein domain with an “RNA-protein interaction domain” (aka “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a split prime editor, as well as to recruitment additional functionalities to a split prime editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplary scenario a deaminase-MS2 fusion can recruit a Cas9-MCP fusion.

The adaptor protein may utilize known linkers to attach such functional domains. The adaptor protein may be any number of proteins that binds to an aptamer or recognition site introduced into the modified sgRNA and which allows proper positioning of one or more functional domains, once the sgRNA has been incorporated into the CRISPR complex, to affect the target with the attributed function. Such adapter proteins may be coat proteins (e.g., bacteriophage coat proteins). The functional domains associated with such adaptor proteins (e.g., in the form of fusion protein) may include, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g., light inducible).

In some embodiments, the prime editor system further comprises a scaffold protein that has affinity for the first polypeptide and/or the second polypeptide. In certain embodiments, the scaffold protein is fused to the first polypeptide or the second polypeptide. In certain embodiments, the scaffold protein is not fused to either the first polypeptide or the second polypeptide. In some embodiments, the prime editor system further comprises a second scaffold protein that has affinity for the scaffold protein. In some embodiments, the second scaffold protein has affinity for the first polypeptide. In some embodiments, the second scaffold protein has affinity for to the second polypeptide. In certain embodiments, the second scaffold protein is fused to the first polypeptide or the second polypeptide. In certain embodiments, the second scaffold protein is not fused to either the first polypeptide or the second polypeptide. In some embodiments, the first polypeptide has affinity for an endogenous protein in a host cell. In some embodiments, the second polypeptide has affinity for the endogenous protein in a host cell. In certain embodiments, the first polypeptide has affinity for a first endogenous protein in a host cell and the second polypeptide has affinity for a second endogenous protein in a host cell, and the first endogenous protein has affinity for the second endogenous protein. In some embodiments, the first polypeptide is configured to become covalently attached to the second polypeptide in a host cell.

In some aspects, provided herein are prime editing system that include modified PEgRNAs. In some embodiments, the PEgRNA associates with and directs a split prime editor to incorporate the one or more (e.g., two or more, three or more, four or more, or five or more) intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a split prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm. In certain embodiments, the PERNA comprises a primer binding site sequence (PBS) that can initiate target-primed DNA synthesis. In some embodiments, the PEgRNA comprises an editing template that comprises one or more intended nucleotide edits to be incorporated in the target gene by prime editing. In some embodiments, the extension arm comprises a PBS. In some embodiments, the extension arm comprises an editing template that comprises one or more intended nucleotide edits to be incorporated in the target gene by prime editing.

A “primer binding site” (PBS or primer binding site sequence) is a single-stranded portion of the PEgRNA that comprises a region of complementarity to the PAM strand (i.e. the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a split prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PERNA is a single-stranded portion of the PEgRNA that is 5′ of the PBS and comprises a region of complementarity to the PAM strand (i.e. the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PERNA comprising both the PBS and the editing template is complementary or substantially complementary to an endogenous sequence on the PAM strand (i.e. the non-target strand or the edit strand) of the double stranded target DNA except for one or more non-complementary nucleotides at the intended nucleotide edit positions. As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits.

### Spacers

A spacer may guide a prime editing complex to a genomic locus with identical or substantially identical sequence during prime editing. In some embodiments, the PERNA comprises a spacer. In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length.

In some embodiments, a spacer sequence comprises a region that has substantial complementarity to a search target sequence on the target strand of a double stranded target DNA. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U)nucleobase of the PEgRNA or guide RNA or any chemically modified uracil nucleobase known in the art, such as 5-methoxyuracil.

### Primer Binding Site (PBS)

A PERNA may comprise a primer binding site (PBS) and an editing template (e.g., an RTT). The extension arm of a PEgRNA may comprise a PBS and an editing template. In some embodiments, a PBS may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that hybridizes with a free 3′ end of a single stranded DNA in the target gene generated by nicking with a split prime editor. The length of the PBS sequence may vary depending on, e.g., the split prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 6 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length.

The PBS may be complementary or substantially complementary to a DNA sequence in the edit strand of the target gene. By annealing with the edit strand at a free hydroxy group, e.g., a free 3′ end generated by split prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene. In some embodiments, the PBS is perfectly complementary, or 100% complementary, to a region of the edit strand of the target gene.

An extension arm of a PERNA may comprise an editing template that serves as a DNA synthesis template for the DNA polymerase in a split prime editor during prime editing.

The length of an editing template may vary depending on, e.g., the split prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated into the target gene. In some embodiments, the editing template comprises a nucleotide sequence comprising about 85% to about 95% complementarity to an editing target sequence in the edit strand in the target gene. In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PERNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a split prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. For example, in some embodiments, a PEgRNA comprises, from 5′ to 3′: a spacer, a gRNA core, an editing template, and a PBS. In some embodiments, a PERNA comprises, from 5′ to 3′: an editing template, a PBS, a spacer, and a gRNA core. In some embodiments, the PBS and/or the editing template is positioned within the gRNA core, i.e., flanked by a first half of the gRNA core and a second half of the gRNA core.

In certain embodiments, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, wherein the PEgRNA further comprises one or more nucleic acid moieties at its 3′ end.

In some embodiments, the PEgRNA comprises, in 5′ to 3′ order, the spacer, the gRNA core, the editing template, and the PBS.

In certain embodiments, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, wherein the gRNA core comprises one or more sequence modifications compared to SEQ ID NO. 16.

In some embodiments, the PEgRNA comprises, in 5′ to 3′ order, the spacer, the gRNA core, the editing template, and the PBS.

In certain embodiments, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, and v) a tag sequence that comprises a region of complementarity to the PBS and/or the editing template.

In some embodiments, the PEgRNA comprises, in 5′ to 3′ order, the spacer, the gRNA core, the editing template, the PBS, and the tag sequence.

In some embodiments, the PEgRNA comprises, in 5′ to 3′ order, the editing template, the PBS, the tag sequence, the spacer, and the gRNA core.

In certain embodiments, PEgRNAs provided herein comprise in 5′ to 3′ order: i) a spacer that comprises a region of complementarity to a search target sequence in target strand of a double stranded target DNA; ii) 5′ part of a guide RNA (gRNA) core; iii) an editing template that comprises an intended edit compared to the double stranded target DNA; iv) a primer binding site (PBS) that comprises a region of complementarity to a region upstream of a nick site in a non-target strand of the double stranded target DNA; and v) a 3′ part of a gRNA core. In some embodiments, the 5′ part of the gRNA core and the 3′ part of the gRNA core form a complete functional gRNA core that can associate with a programmable DNA binding protein of a split prime editor, e.g., a Cas9 nickase. In some embodiments, the 5′ part of the gRNA core comprises a direct repeat, a first stem loop, and a 5′ half of a second stem loop. In some embodiments, the 3′ part of the gRNA core comprises a 3′ half of a second stem loop and a third stem loop. In some embodiments, the PEgRNA further comprises a tag sequence that comprises a region of complementarity to the PBS and/or the editing template.

In certain embodiments, PEgRNAs provided herein comprise: i) a first sequence comprising a spacer that comprises a region of complementarity to a search target sequence in target strand of a double stranded target DNA, and a first half of a gRNA core; and ii) a second sequence comprising a second half of the gRNA core, an editing template that comprises an intended edit compared to the double stranded target DNA; a primer binding site (PBS) that comprises a region of complementarity to a region upstream of a nick site in a non-target strand of the double stranded target DNA; and, wherein the gRNA core comprises a direct repeat, a first stem loop, and a second stem loop. In certain embodiments, PEgRNAs provided herein comprise i) a first sequence comprising an editing template that comprises an intended edit compared to the double stranded target DNA; a primer binding site (PBS) that comprises a region of complementarity to a region upstream of a nick site in a non-target strand of the double stranded target DNA; a spacer that comprises a region of complementarity to a search target sequence in target strand of a double stranded target DNA; and a first half of a gRNA core; and ii) a second sequence comprising a second half of a gRNA core, wherein the gRNA core comprises a direct repeat, a first stem loop, and a second stem loop. In some embodiments, the first half of the gRNA core comprises a direct repeat, a first stem loop, and a 5′ half of a second stem loop. In some embodiments, the second part of the gRNA core comprises a 3′ half of a second stem loop and a third stem loop. In some embodiments, the first half of the gRNA core comprises a first half of a direct repeat. In some embodiments, the second half of the gRNA core comprises a second half of a direct repeat, a first stem loop, a second stem loop, and a third stem loop.

In some embodiments, the first sequence is on a first molecule and the second sequence is on a second molecule.

In some embodiments, the first sequence and the second sequence are on the same molecule.

Provided herein in some embodiments are example sequences for PEgRNA spacers, PBS, RTT, and ngRNA spacers for a prime editing system comprising a nuclease that recognizes the PAM sequence “NGG.” In some embodiments, a PAM motif on the edit strand comprises an “NGG” motif, wherein N is any nucleotide. In some embodiments, a PEgRNA of this disclosure is part of a prime editing system that recognizes the PAM motif CGG. In some embodiments, a PERNA of this disclosure is part of a prime editing system that recognizes the PAM motif AGG.

Modified gRNA Cores

In some embodiments, a gRNA core of a PEgRNA associates with a programmable DNA binding domain in a split prime editor. In some embodiments, the gRNA core comprises a direct repeat, a first stem loop, and a second stem loop. In some embodiments, the gRNA core further comprises a third stem loop. A guide RNA core (also referred to herein as the gRNA core, gRNA scaffold, or gRNA backbone sequence) of a PEgRNA may contain a polynucleotide sequence that binds to a DNA binding domain (e.g., Cas9) of a split prime editor. The gRNA core may interact with a split prime editor as described herein, for example, by association with a DNA binding domain, such as a DNA nickase of the split prime editor.

One of skill in the art will recognize that different split prime editors having different DNA binding domains from different DNA binding proteins may require different gRNA core sequences specific to the DNA binding protein. In some embodiments, the gRNA core is capable of binding to a Cas9-based split prime editor. In some embodiments, the gRNA core is capable of binding to a Cpf1-based split prime editor. In some embodiments, the gRNA core is capable of binding to a Cas12b-based split prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired regions. In some embodiments, a gRNA core capable of binding to a Cas9 comprises, from 5′ to 3′: a repeat sequence, a loop structure, an antirepeat sequence, a first stem loop, a second stem loop, and a third stem loop. As used herein, a repeat sequence and an antirepeat sequence refer to the nucleic acid secondary structure formed by the direct repeat region, formed by base pairing between sequences equivalent to the crRNA and tracrRNA of a Cas9 guide RNA. The repeat sequence and the antirepeat sequence may be connected by a loop structure, and the secondary structure formed by base pairing between the repeat and antirepeat sequence may be referred to as the direct repeat region (alternatively, the repeat, antirepeat, and the connecting loop structure may be referred to as the tetraloop). In some embodiments, the direct repeat region of the gRNA core comprises one or more base paired regions: a base paired “lower stem” adjacent to the spacer sequence and a base paired “upper stem” following the lower stem, where the lower stem and upper stem may be connected by a “bulge” comprising unpaired RNAsAs used herein, positions of alterations to the gRNA core may be referred to in the context of the secondary structure of the gRNA core. For example, a “first base pair in the direct repeat (or lower stem)” refers to the base pair between the 5′ most nucleotide in the repeat sequence and the complementary nucleotide that is the 3′ most nucleotide in the antirepeat sequence, and a “second base pair in the direct repeat (or lower stem)” refers to the base pair between the second 5′ most nucleotide in the repeat sequence and the complementary nucleotide in the antirepeat sequence. Similarly, the “start” or “beginning” base pair of a second stem loop refers to the base pair formed between the 5′ most nucleotide in the second stem loop and the complementary nucleotide in the complementary portion of the second stem loop. The “end” or “last” base pair of a second stem loop refers to, wherein the second stem loop is formed by base pairing of a 5′ portion of the stem and a 3′ portion of the stem connected by a loop, the base pair formed between the 3′ most nucleotide in the 5′ portion of the stem and the complementary nucleotide in the complementary 3′ portion of the stem.

The gRNA core may further comprise, 3′ to the direct repeat, a first stem loop, a second stem loop, and a third stem loop. In some embodiments, the gRNA core may comprise a direct repeat, and at least one, at least two, or at least three stem loops. As used herein, a stem loop (or a hairpin loop) is base pairing pattern that can occur in single-stranded nucleic acids. In some embodiments, a stem loop may be formed when two regions of the same nucleic acid strand are at least partially complementary in nucleotide sequence when read in opposite directions, therefore, the base-pairs can form a double helix that comprises an unpaired loop. Stem loops within a gRNA core described herein may be numbered starting from the 5′ to the 3′ end of the gRNA core. For example, the “first stem loop” would be the first stem loop (not including any direct repeats) at the 5′ end proximal to the direct repeat of the gRNA core sequence. A “second stem loop” would be the second stem loop (not including any direct repeats) following the first stem loop in a 5′ to 3′ direction, and so on.

In some embodiments, the gRNA core comprises nucleotide alterations as compared to a wild type gRNA core. For example, in some embodiments, one or more nucleotides in the gRNA core is deleted, inserted, and/or substituted as compared to a wild type gRNA core. In some embodiments, the gRNA core of a PEgRNA is capable of binding to a Cas9 (e.g. nCas9) in a split prime editor, and comprise one or more nucleotide alterations or modifications as compared to a wild type CRISPR-Cas9 guide RNA scaffold. In some embodiments, the gRNA core comprises one or more nucleotide insertions, deletions, and/or substitutions in the direct repeat as compared to a wild type CRISPR-Cas9 guide RNA scaffold. In some embodiments, the gRNA core comprises one or more nucleotide insertions, deletions, and/or substitutions in the lower stem or upper stem of the direct repeat. In some embodiments, the gRNA core comprises one or more nucleotide substitutions in the lower stem of the direct repeat. In some embodiments, the gRNA core comprises one or more nucleotide insertions in the upper stem of the direct repeat. In some embodiments, the gRNA core comprises one or more nucleotide insertions, deletions, and/or substitutions in the first stem loop as compared to a wild type CRISPR-Cas9 guide RNA scaffold. In some embodiments, the gRNA core comprises one or more nucleotide insertions, deletions, and/or substitutions in the second stem loop as compared to a wild type CRISPR-Cas9 guide RNA scaffold. In some embodiments, the gRNA core comprises one or more nucleotide insertions in the second stem loop. In some embodiments, the gRNA core comprises one or more nucleotide insertions, deletions, and/or substitutions in the third stem loop as compared to a wild type CRISPR-Cas9 guide RNA scaffold. In some embodiments, the gRNA core comprises one or more nucleotide insertions, deletions, and/or substitutions as compared to a wild type CRISPR-Cas9 guide RNA scaffold, and comprises a third stem loop that has the same sequence as the third stem loop of a wild type CRISPR-Cas9 guide RNA scaffold.

In some embodiments, RNA nucleotides in the lower stem, upper stem, an/or the stem loop regions may be replaced with one or more DNA sequences. In some embodiments, the gRNA core comprises unmodified or wild type RNA sequences in the nexus and/or the bulge regions. In some embodiments, the gRNA core does not include long stretches of A-U pairs, for example, a GUUUU-AAAAC pairing element.

In some embodiments, the PEgRNA comprises a guide RNA (gRNA) core that associates with a DNA binding domain, e.g., a CRISPR-Cas protein domain, of a prime editor. In some embodiments, the PEgRNA comprises a guide RNA (gRNA) core that associates with a DNA binding domain, e.g., a Cas9 domain, of a split prime editor. In certain aspects, the gRNA core of the PEgRNAs provided herein comprises one or more sequence modifications compared to SEQ ID NO. 16. In some embodiments, the one or more (e.g., two or more, three or more, four or more, or five or more) sequence modifications comprises a gRNA core difference. In some embodiments, the gRNA core comprises a sequence selected from SEQ ID NOs: 16-61. In some embodiments, the gRNA core comprises a first gRNA core sequence comprising a 5′ half of the gRNA core and a second gRNA core sequence comprising a 3′ half of the gRNA core, and wherein the PEgRNA comprises, in 5′ to 3′ order: the spacer, the first gRNA core sequence, the editing template, the PBS, the tag sequence, and the second gRNA core sequence. The 5′ half and the 3′ half can form a functional gRNA core for association/binding with a programmable DNA binding protein, e.g., a Cas protein. One of skill in the art will recognize that different split prime editors having different DNA binding domains from different DNA binding proteins may require different gRNA core sequences specific to the DNA binding protein. In some embodiments, the gRNA core is capable of binding to a Cas9-based split prime editor. In some embodiments, the gRNA core is capable of binding to a Cpf1-based split prime editor. In some embodiments, the gRNA core is capable of binding to a Cas12b-based split prime editor.

In some embodiments, the gRNA core of the PEgRNAs provided herein comprises one or more sequence modifications compared to SEQ ID NO. 16. In some embodiments, the one or more sequence modifications comprises a gRNA core alteration compared to a Cas9 guide RNA scaffold (e.g., SEQ ID No.: 16).

In some embodiments, the one or more sequence modifications comprises a sequence modification in the direct repeat. In some embodiments, sequence modification in the gRNA core of a PERNA comprises one or more nucleotide flips. As used herein, the term “flip” refers to the modification of a sequence such that nucleotide bases that that base-pair with each other in the stem of a loop or hairpin structure are exchanged for each other. For example, an original unmodified stem structure may comprise an A/U base pair, with A in a first strand (or region) and U in the complementary strand (or region) of the stem structure. An A/U to U/A base pair flip substitutes the Adenosine in the first strand (or region) with a Uracil and substitutes the Uracil in the complementary strand (or region) with an Adenosine, thereby “flipping” the A/U base pair to an U/A base pair. In some embodiments, a flip of nucleotides can be used, for example, to break-up sequences containing repeats of the same base (for example sequences of at least 3, 4, 5, 6, or 7 consecutive A nucleotides, U nucleotides, C nucleotides, or G nucleotides) present in a nucleic acid molecule without disrupting its secondary structure. In some embodiments, instead of a flip, the original base pair is replaced with an alternative base pair (e.g., an A/U base pair is replaced with a C/G or G/C base pair).

In some embodiments, the direct repeat of the gRNA core may comprise at least one flip of an A-U base pair in a lower stem of the direct repeat, optionally wherein the lower stem does not contain 2, 3, 4, or more contiguous A-U base pairs; and/or at least one flip of an A/U base pair in the direct repeat comprises a flip of the fourth A/U base pair in the lower stem of the direct repeat.

In some embodiments, the sequence modification in the direct repeat comprises insertion of one or more nucleotides in the upper stem of the direct repeat of the gRNA core, thereby resulting in an extension of the upper stem as compared to a wild type gRNA core, e.g., as set forth in SEQ ID NO: 16. The extension in the upper stem may be from 1, 2, 3, 4, 5, 6, 7, 8, 9, 10, 11, 12, 13, 14, 15, or 16 base pairs. In some embodiments, the gRNA core comprises a sequence selected from SEQ ID NOs: 26-37.

In some embodiments, the one or more sequence modifications comprises a sequence modification in the second stem loop.

In some embodiments, the modification in the second stem loop comprises a flip of a G/C base pair. In some embodiments, the modification in the second stem loop comprises a flip of an A/U base pair in the second stem loop. In some embodiments, the modification in the second stem loop comprises substitution of a A/U base pair with a G/C base pair. In some embodiments, the modification in the second stem loop comprises substitution of a U/A base pair with a G/C base pair. In some embodiments, the modification in the second stem loop comprises substitution of a A/U base pair with a G/C base pair, and further comprises a substitution of a U/A base pair with a G/C base pair. In some embodiments, the gRNA core comprises a nucleic acid sequence selected from SEQ ID NOs: 21, 22 or 25. Exemplary gRNA core sequences and sequence modifications are shown in Table 5. In some embodiments, the gRNA core comprises a sequence selected from SEQ ID NOs: 16-61.

In some embodiments, the one or more sequence modifications comprises a modification in a third stem loop of the gRNA core. In some embodiments, the modification in the third stem loop comprises a flip of a G/C base pair. In some embodiments, the modification in the third stem loop comprises a flip of an A/U base pair.

The gRNA core may comprise any one of modifications described in Table 5 or any combination thereof.

In some embodiments, the gRNA core has a flipped 1st A-U base pair in the direct repeat. In some embodiments, the gRNA core has a flipped 2nd A-U base in the direct repeat. In some embodiments, the gRNA core has a flipped 3rd A-U base pair in the direct repeat. In some embodiments, the gRNA core has a flipped 4th A-U base pair in the direct repeat.

In some embodiments, the gRNA core comprises a substitution of an A-U base pair (bp) with a G-C Bp at the fourth base pair of the second stem loop. In some embodiments, the gRNA core comprises a substitution of an A-U Bp with a C-G Bp at the fourth base pair of second stem loop.

In some embodiments, the gRNA core comprises a five base pair extension of the upper stem of the direct repeat (tgctg and cagca). In some embodiments, the gRNA has a “flip and extension” (M4 and E5), as described in Nelson, J. W., Randolph, P. B., Shen, S. P. et al. Engineered pegRNAs improve prime editing efficiency. Nat Biotechnol (2021). The M4 modification is flipping the 4th A-U base pair in the direct repeat of gRNA core. The E5 modification is extending the end of the upper stem of the direct repeat with a five bp sequence (tgctg and cagca).

In some embodiments, a gRNA core comprises a M4 modification. In some embodiments, a gRNA core comprises a E5 modification. In some embodiments, a gRNA core comprises a M4 modification and a E5 modification.

In some embodiments, a gRNA core comprises a substitution of a A/U base pair with a G/C base pair in the second stem loop. In some embodiments, the gRNA core comprises a substitution of a A/U base pair with a G/C base pair at the first base pair of the second stem loop.

In some embodiments, the gRNA core has a 1 base pair extension in the upper stem of the direct repeat sequence (c and g). In some embodiments, the gRNA core has a 2 base pair extension in the upper stem of the direct repeat sequence (cc and gg). In some embodiments, the gRNA core has a 2 base pair extension in the upper stem of the direct repeat sequence (ca and tg). In some embodiments, the gRNA core has a 2 base pair extension in the upper stem of the direct repeat sequence (cg and tg). In some embodiments, the gRNA core has a 1 base pair extension in the upper stem of the direct repeat sequence (a and t). In some embodiments, the gRNA core has a 2 base pair extension in the upper stem of the direct repeat sequence (ac and gt). In some embodiments, the gRNA core has a 2 base pair extension in the upper stem of the direct repeat sequence (aa and tt). In some embodiments, the gRNA core has a 2 base pair extension in the upper stem of the direct repeat sequence (ag and tt). In some embodiments, the gRNA core has a 3 base pair extension in the upper stem of the direct repeat sequence (ccc and ggg). In some embodiments, the gRNA core has a 4 base pair extension in the upper stem of the direct repeat sequence (ccac and gtgg). In some embodiments, the gRNA core has a 5 base pair extension in the upper stem of the direct repeat sequence (ccaac and gttgg). In some embodiments, the gRNA core has a 6 base pair extension in the upper stem of the direct repeat sequence (ccacac and gtgtgg).

In some embodiments, the gRNA core has a 1 base pair extension in the second stem loop sequence (c and g). In some embodiments, the gRNA core has a 2 base pair extension in the second stem loop sequence (cc and gg). In some embodiments, the gRNA core has a 2 base pair extension in the second stem loop sequence (ca and tg). In some embodiments, the gRNA core has a 2 base pair extension in the second stem loop sequence (cg and tg). In some embodiments, the gRNA core has a 1 base pair extension in the second stem loop sequence (a and t). In some embodiments, the gRNA core has a 2 base pair extension in the second stem loop sequence (ac and gt). In some embodiments, the gRNA core has a 2 base pair extension in the second stem loop sequence (aa and tt). In some embodiments, the gRNA core has a 2 base pair extension in the second stem loop sequence (ag and tt). In some embodiments, the gRNA core has a 3 base pair extension in the second stem loop sequence (ccc and ggg). In some embodiments, the gRNA core has a 4 base pair extension in the second stem loop sequence (ccac and gtgg). In some embodiments, the gRNA core has a 5 base pair extension in the second stem loop sequence (ccaac and gttgg). In some embodiments, the gRNA core has a 6 base pair extension in the second stem loop sequence (ccacac and gtgtgg).

In some embodiments, the gRNA core has a 1 base pair extension in the third stem loop sequence (c and g). In some embodiments, the gRNA core has a 2 base pair extension in the third stem loop sequence (cc and gg). In some embodiments, the gRNA core has a 2 base pair extension in the third stem loop sequence (ca and tg). In some embodiments, the gRNA core has a 2 base pair extension in the third stem loop sequence (cg and tg). In some embodiments, the gRNA core has a 1 base pair extension in the third stem loop sequence (a and t). In some embodiments, the gRNA core has a 2 base pair extension in the third stem loop sequence (ac and gt). In some embodiments, the gRNA core has a 2 base pair extension in the third stem loop sequence (aa and tt). In some embodiments, the gRNA core has a 2 base pair extension in the third stem loop sequence (ag and tt). In some embodiments, the gRNA core has a 3 base pair extension in the third stem loop sequence (ccc and ggg). In some embodiments, the gRNA core has a 4 base pair extension in the third stem loop sequence (ccac and gtgg). In some embodiments, the gRNA core has a 5 base pair extension in the third stem loop sequence (ccaac and gttgg). In some embodiments, the gRNA core has a 6 base pair extension in the third stem loop sequence (ccacac and gtgtgg).

In some embodiments, as compared to editing efficiency with a control PEgRNA having a gRNA core without modifications, a gRNA core modification increase efficiency of editing by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%. Exemplary nucleotide sequence modifications in the gRNA core of a PEgRNA are provided in Table 5. Modifications compared to a wild type Cas9 gRNA scaffold sequence are shown in lower case letters.

TABLE 5
Exemplary gRNA Core Sequences

| SEQ | gRNA | |
|---|---|---|
| ID | Core | |
| NO. | name | gRNA Core Sequence |
| 16 | wild type | GTTTTAGAGCTAGAAATAGCAAGTTAAAATAAGGCTAGTCCGTTATCAACTTGAA |
| | Cas9 | AAAGTGGCACCGAGTCGGTGC |
| | guide | |
| | RNA | |
| | scaffold | |
| 17 | M1 | GaTTTAGAGCTAGAAATAGCAAGTTAAAtTAAGGCTAGTCCGTTATCAACTTGAAA |
| | | AAGTGGCACCGAGTCGGTGC |
| 18 | M2 | GTaTTAGAGCTAGAAATAGCAAGTTAAtATAAGGCTAGTCCGTTATCAACTTGAAA |
| | | AAGTGGCACCGAGTCGGTGC |
| 19 | M3 | GTTaTAGAGCTAGAAATAGCAAGTTAtAATAAGGCTAGTCCGTTATCAACTTGAAA |
| | | AAGTGGCACCGAGTCGGTGC |
| 20 | M4 | GTTTaAGAGCTAGAAATAGCAAGTTtAAATAAGGCTAGTCCGTTATCAACTTGAAA |
| | | AAGTGGCACCGAGTCGGTGC |
| 21 | sl2 gc | GTTTTAGAGCTAGAAATAGCAAGTTAAAATAAGGCTAGTCCGTTATCAACTgGAA |
| | | AcAGTGGCACCGAGTCGGTGC |
| 22 | sl2 cg | GTTTTAGAGCTAGAAATAGCAAGTTAAAATAAGGCTAGTCCGTTATCAACTcGAA |
| | | AgAGTGGCACCGAGTCGGTGC |
| 23 | E5 | GTTTTAGAGCTAtgctgGAAAcagcaTAGCAAGTTAAAATAAGGCTAGTCCGTTATCAA |
| | | CTTGAAAAAGTGGCACCGAGTCGGTGC |
| 24 | F+E | GTTTaAGAGCTAtgctgGAAAcagcaTAGCAAGTTtAAATAAGGCTAGTCCGTTATCAAC |
| | | TTGAAAAAGTGGCACCGAGTCGGTGC |
| 25 | s12_flip | GTTTAAGAGCTAGAAATAGCAAGTTTAAATAAGGCTAGTCCGTTATCAGCGTGAA |
| | | AACGCGGCACCGAGTCGGTGC |
| 26 | TetraLoop_ | GTTTAAGAGCTAcGAAAgTAGCAAGTTTAAATAAGGCTAGTCCGTTATCAACTTGA |
| | L0 | AAAAGTGGCACCGAGTCGGTGC |
| 27 | TetraLoop_ | GTTTAAGAGCTAccGAAAggTAGCAAGTTTAAATAAGGCTAGTCCGTTATCAACTT |
| | L1 | GAAAAAGTGGCACCGAGTCGGTGC |
| 28 | TetraLoop_ | GTTTAAGAGCTAcaGAAAtgTAGCAAGTTTAAATAAGGCTAGTCCGTTATCAACTTG |
| | L2 | AAAAAGTGGCACCGAGTCGGTGC |
| 29 | TetraLoop_ | GTTTAAGAGCTAcgGAAAtgTAGCAAGTTTAAATAAGGCTAGTCCGTTATCAACTTG |
| | L3 | AAAAAGTGGCACCGAGTCGGTGC |
| 30 | TetraLoop_ | GTTTAAGAGCTAaGAAAtTAGCAAGTTTAAATAAGGCTAGTCCGTTATCAACTTGA |
| | L4 | AAAAGTGGCACCGAGTCGGTGC |
| 31 | TetraLoop_ | GTTTAAGAGCTAacGAAAgtTAGCAAGTTTAAATAAGGCTAGTCCGTTATCAACTTG |
| | L5 | AAAAAGTGGCACCGAGTCGGTGC |
| 32 | TetraLoop_ | GTTTAAGAGCTAaaGAAAttTAGCAAGTTTAAATAAGGCTAGTCCGTTATCAACTTG |
| | L6 | AAAAAGTGGCACCGAGTCGGTGC |
| 33 | TetraLoop_ | GTTTAAGAGCTAagGAAAttTAGCAAGTTTAAATAAGGCTAGTCCGTTATCAACTTG |
| | L7 | AAAAAGTGGCACCGAGTCGGTGC |
| 34 | TetraLoop_ | GTTTAAGAGCTAcccGAAAgggTAGCAAGTTTAAATAAGGCTAGTCCGTTATCAACT |
| | L8 | TGAAAAAGTGGCACCGAGTCGGTGC |
| 35 | TetraLoop_ | GTTTAAGAGCTAccacGAAAgtggTAGCAAGTTTAAATAAGGCTAGTCCGTTATCAAC |
| | L9 | TTGAAAAAGTGGCACCGAGTCGGTGC |
| 36 | TetraLoop_ | GTTTAAGAGCTAccaacGAAAgttggTAGCAAGTTTAAATAAGGCTAGTCCGTTATCAA |
| | L10 | CTTGAAAAAGTGGCACCGAGTCGGTGC |
| 37 | TetraLoop_ | GTTTAAGAGCTAccacacGAAAgtgtggTAGCAAGTTTAAATAAGGCTAGTCCGTTATC |
| | L11 | AACTTGAAAAAGTGGCACCGAGTCGGTGC |
| 38 | Loop2_L0 | GTTTAAGAGCTAGAAATAGCAAGTTTAAATAAGGCTAGTCCGTTATCAACTTcGA |
| | | AAgAAGTGGCACCGAGTCGGTGC |
| 39 | Loop2_L1 | GTTTAAGAGCTAGAAATAGCAAGTTTAAATAAGGCTAGTCCGTTATCAACTTccGA |
| | | AAggAAGTGGCACCGAGTCGGTGC |
| 40 | Loop2_L2 | GTTTAAGAGCTAGAAATAGCAAGTTTAAATAAGGCTAGTCCGTTATCAACTTcaGA |
| | | AAtgAAGTGGCACCGAGTCGGTGC |
| 41 | Loop2_L3 | GTTTAAGAGCTAGAAATAGCAAGTTTAAATAAGGCTAGTCCGTTATCAACTTcgGA |
| | | AAtgAAGTGGCACCGAGTCGGTGC |
| 42 | Loop2_L4 | GTTTAAGAGCTAGAAATAGCAAGTTTAAATAAGGCTAGTCCGTTATCAACTTaGA |
| | | AAtAAGTGGCACCGAGTCGGTGC |
| 43 | Loop2_L5 | GTTTAAGAGCTAGAAATAGCAAGTTTAAATAAGGCTAGTCCGTTATCAACTTacGA |
| | | AAgtAAGTGGCACCGAGTCGGTGC |
| 44 | Loop2_L6 | GTTTAAGAGCTAGAAATAGCAAGTTTAAATAAGGCTAGTCCGTTATCAACTTaaGA |
| | | AAttAAGTGGCACCGAGTCGGTGC |
| 45 | Loop2_L7 | GTTTAAGAGCTAGAAATAGCAAGTTTAAATAAGGCTAGTCCGTTATCAACTTagGA |
| | | AAttAAGTGGCACCGAGTCGGTGC |
| 46 | Loop2_L8 | GTTTAAGAGCTAGAAATAGCAAGTTTAAATAAGGCTAGTCCGTTATCAACTTcccG |
| | | AAAgggAAGTGGCACCGAGTCGGTGC |
| 47 | Loop2_L9 | GTTTAAGAGCTAGAAATAGCAAGTTTAAATAAGGCTAGTCCGTTATCAACTTccacG |
| | | AAAgtggAAGTGGCACCGAGTCGGTGC |
| 48 | Loop2_L10 | GTTTAAGAGCTAGAAATAGCAAGTTTAAATAAGGCTAGTCCGTTATCAACTTecaac |
| | | GAAAgttggAAGTGGCACCGAGTCGGTGC |
| 49 | Loop2_L11 | GTTTAAGAGCTAGAAATAGCAAGTTTAAATAAGGCTAGTCCGTTATCAACTTecaca |
| | | cGAAAgtgtggAAGTGGCACCGAGTCGGTGC |
| 50 | Loop3_L0 | GTTTAAGAGCTAGAAATAGCAAGTTTAAATAAGGCTAGTCCGTTATCAACTTGAA |
| | | AAAGTGGCACCGcAGTgCGGTGC |
| 51 | Loop3_L1 | GTTTAAGAGCTAGAAATAGCAAGTTTAAATAAGGCTAGTCCGTTATCAACTTGAA |
| | | AAAGTGGCACCGccAGTggCGGTGC |
| 52 | Loop3_L2 | GTTTAAGAGCTAGAAATAGCAAGTTTAAATAAGGCTAGTCCGTTATCAACTTGAA |
| | | AAAGTGGCACCGcaAGTtgCGGTGC |
| 53 | Loop3_L3 | GTTTAAGAGCTAGAAATAGCAAGTTTAAATAAGGCTAGTCCGTTATCAACTTGAA |
| | | AAAGTGGCACCGcgAGTtgCGGTGC |
| 54 | Loop3_L4 | GTTTAAGAGCTAGAAATAGCAAGTTTAAATAAGGCTAGTCCGTTATCAACTTGAA |
| | | AAAGTGGCACCGaAGTtCGGTGC |
| 55 | Loop3_L5 | GTTTAAGAGCTAGAAATAGCAAGTTTAAATAAGGCTAGTCCGTTATCAACTTGAA |
| | | AAAGTGGCACCGacAGTgtCGGTGC |
| 56 | Loop3_L6 | GTTTAAGAGCTAGAAATAGCAAGTTTAAATAAGGCTAGTCCGTTATCAACTTGAA |
| | | AAAGTGGCACCGaaAGTttCGGTGC |
| 57 | Loop3_L7 | GTTTAAGAGCTAGAAATAGCAAGTTTAAATAAGGCTAGTCCGTTATCAACTTGAA |
| | | AAAGTGGCACCGagAGTttCGGTGC |
| 58 | Loop3_L8 | GTTTAAGAGCTAGAAATAGCAAGTTTAAATAAGGCTAGTCCGTTATCAACTTGAA |
| | | AAAGTGGCACCGcccAGTgggCGGTGC |
| 59 | Loop3_L9 | GTTTAAGAGCTAGAAATAGCAAGTTTAAATAAGGCTAGTCCGTTATCAACTTGAA |
| | | AAAGTGGCACCGccacAGTgtggCGGTGC |
| 60 | Loop3_L10 | GTTTAAGAGCTAGAAATAGCAAGTTTAAATAAGGCTAGTCCGTTATCAACTTGAA |
| | | AAAGTGGCACCGccaacAGTgttggCGGTGC |
| 61 | Loop3_L11 | GTTTAAGAGCTAGAAATAGCAAGTTTAAATAAGGCTAGTCCGTTATCAACTTGAA |
| | | AAAGTGGCACCGccacacAGTgtgtggCGGTGC |

### Nucleic Acid Moieties

In some embodiments, the PEgRNA comprises one or more nucleic acid moieties (e.g., hairpin, pseudoknot, quadruplex, tRNA sequence, aptamer) in addition to the spacer, gRNA core, primer binding site, and editing template. In some embodiments such nucleic acid moieties are positioned on the 3′ end of the PEgRNA.

In some embodiments, the nucleic acid moiety comprise a hairpin. In some embodiments, a hairpin is a nucleic acid secondary structure formed by intramolecular base pairing between a two regions of the same strand, which are typically complementary in nucleotide sequence when read in opposite directions. The two regions base-pair to form a double helix that ends in an unpaired loop. As described herein, the hairpin may be between 5 and 50 nucleotides in length, between 10 and 40 nucleotides in length, or at least 15 and 30 nucleotides in length. The hairpin may be at least 10 nucleotides in length, at least 15 nucleotides in length, at least 20 nucleotides in length, at least 25 nucleotides in length, or at least 30 nucleotides in length. In some embodiments, the hairpin is 14 nucleotides in length. In some embodiments, the hairpin is 18 nucleotides in length. In some embodiments, the hairpin is 22 nucleotides in length. In some embodiments, the hairpin comprises 2, 3, 4, 5, 6, 7, 8, 9, 10, 11, 12, 13, 14, 15 or more contiguous complementary base pairs. In some embodiments, the hairpin comprises 4, 5, 6, 7, 8, 9, or 10 contiguous complementary base pairs. In some embodiments, the hairpin comprises 4-8 contiguous complementary base pairs. In some embodiments, the hairpin comprises 5 contiguous complementary base pairs. In some embodiments, the hairpin comprises 7 contiguous complementary base pairs.

In some embodiments, the nucleic acid moiety comprises a pseudoknot. As used herein, a pseudoknot, includes, but is not limited to a nucleic acid secondary structure containing at least two stem-loop structures in which half of one stem is intercalated between the two halves of another stem. Several distinct folding topologies of pseudoknots exist, including, for example, the H type. In the H-type fold, the bases in the loop of a hairpin form intramolecular pairs with bases outside of the stem. This causes the formation of a second stem and loop, resulting in a pseudoknot with two stems and two loops. As described herein, the pseudoknot may be between 5 and 50 nucleotides in length, between 10 and 40 nucleotides in length, or at least 15 and 30 nucleotides in length. The hairpin may be at least 10 nucleotides in length, at least 15 nucleotides in length, at least 20 nucleotides in length, at least 25 nucleotides in length, or at least 30 nucleotides in length. In some embodiments, the pseudoknot is 22 nucleotides in length.

In some embodiments, the nucleic acid moiety comprises a quadruplex. In some embodiments, quadruplexes are noncanonical four-stranded, nucleic acid secondary structures that can be formed, in some contexts, in guanine-rich or cysteine-rich DNA and RNA sequences. As described herein, the quadruplexes may be between 5 and 50 nucleotides in length, between 10 and 40 nucleotides in length, or at least 15 and 30 nucleotides in length. The hairpin may be at least 10 nucleotides in length, at least 15 nucleotides in length, at least 20 nucleotides in length, at least 25 nucleotides in length, or at least 30 nucleotides in length. In some embodiments, the quadruplex is 18 nucleotides in length. In some embodiments, the quadruplex is rich in Guanine (a G-quadruplex). In some embodiments, the quadruplex is rich in Cytosine (a C-quadruplex).

In some embodiments, the nucleic acid moiety comprises an aptamer. In some embodiments, an aptamer comprises a short, single-stranded nucleic acid oligomer that can bind to a specific target molecule. Aptamers may assume a variety of shapes due to their tendency to form helices and single-stranded loops. As described herein, the aptamer may be between 5 and 50 nucleotides in length, between 10 and 40 nucleotides in length, or at least 15 and 30 nucleotides in length. The hairpin may be at least 10 nucleotides in length, at least 15 nucleotides in length, at least 20 nucleotides in length, at least 25 nucleotides in length, or at least 30 nucleotides in length. In some embodiments, the aptamer is 19 nucleotides in length. In some embodiments, the aptamer is 33 nucleotides in length.

In some embodiments, the nucleic acid moiety comprises a tRNA sequence. A tRNA sequence may be long (e.g., at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, or at least 75 nucleotides) In some embodiments, a tRNA sequence may be short (less than 25 nucleotides, less than 20 nucleotides, less than 15 nucleotides, or less than 10 nucleotides). As described herein, the tRNA sequences may be between 5 and 80 nucleotides in length, between 10 and 70 nucleotides in length, or at least 15 and 60 nucleotides in length. The hairpin may be at least 10 nucleotides in length, at least 15 nucleotides in length, at least 20 nucleotides in length, at least 25 nucleotides in length, at least 30 nucleotides in length, at least 40 nucleotides in length, at least 50 nucleotides in length, at least 60 nucleotides in length, or at least 70 nucleotides in length. In some embodiments, the aptamer is 18 nucleotides in length. In some embodiments, the aptamer is 61 nucleotides in length.

In some embodiments, the RNA scaffold described herein comprises an aptamer that binds to an adapter protein described herein.

Exemplary moieties can be found in Table 7. A person of skill in the art would appreciate that the present disclosure is not limited by the sequences and structures in Table 7 as the configurations in Table 7 are examples of a broader class of moieties included in the present disclosure.

In some embodiments, the one or more nucleic acid moieties comprise a hairpin (e.g., hairpin comprising a region of self-complementarity, optionally wherein the region of self-complementary comprises 2, 3, 4, 5, 6, 7, 8, 9, 10 or more contiguous complementary base pairs), a quadruplex (e.g., a G-quadruplex or a C-quadruplex, optionally wherein the G-quadruplex or the C-quadruplex is derived from a VEGF gene promoter), a tRNA sequence (e.g., a tRNA sequence, optionally wherein the tRNA sequence is a tRNA (Proline) sequence), an aptamer (e.g., an aptamer derived from a viral protein-binding sequence, optionally wherein the aptamer comprises a viral reverse transcriptase recruitment sequence, optionally wherein the aptamer comprises a MS2 protein binding sequence or a Moloney Murine leukemia (MMLV) reverse transcriptase recruitment sequence), and/or a pseudoknot (e.g. pseudoknot is derived form a potato roll leaf virus (PLRV)), or any combination thereof.

In some embodiments, the one or more nucleic acid moieties comprise a structure derived form a replication recognition sequence of a retrovirus. In some embodiments, the nucleic acid moiety comprises a sequence derived from a replication recognition sequence of a Moloney Murine leukemia virus (MMLV). In some embodiments, the one or more nucleic acid moieties comprise a nucleic acid sequence selected from SEQ ID NOs 12-15.

In some embodiments, the one or more nucleic acid moieties comprises a hairpin. In some embodiments, the hairpin comprises a sequence of any one of SEQ ID Nos: 1-3 or 5-7.

In some embodiments, the one or more nucleic acid moieties comprises a pseudoknot. In some embodiments, the pseudoknot is derived from potato roll-leaf virus. In some embodiments, the pseudoknot comprises the sequence of SEQ ID NO: 4. In some embodiments, the one or more nucleic acid moieties comprises a MS2 hairpin. In some embodiments, the nucleotide sequence of the MS2 hairpin (or also referred to as the “MS2 aptamer”) is: GCCAACATGAGGATCACCCATGTCTGCAGGGCC (SEQ ID NO: 4446). In some embodiments, the nucleotide sequence of the MS2 aptamer comprises the sequence of SEQ ID NO: 9. In some embodiments, a MS2 coat protein (MCP) recognizes the MS2 hairpin. In some embodiments, the amino acid sequence of the MCP is:

(SEQ ID NO: 4447)
GSASNFTQFVLVDNGGTGDVTVAPSNFANGVAEWISSNSRSQAYKVTCS
VRQSSAQNRKYTIKVEVPKVATQTVGGEELPVAGWRSYLNMELTIPIFA
TNSDCELIVKAMQGLLKDGNPIPSAIA ANSGIY.

In some embodiments, the one or more nucleic acid moieties comprises a G-quadruplex or a C-quadruplex. In some embodiments, the one or more nucleic acid moieties comprises a quadruplex from a VEGF gene promoter. In some embodiments, the quadruplex comprises the sequence of SEQ ID NO: 10 or 11.

In some embodiments, the PEgRNA comprises one or more nucleic acid moieties at its 3′ end. In some embodiments, the PEgRNA comprises one or more nucleic acid moieties at its 5′ end.

TABLE 6
Exemplary Nucleic Acid Motif Sequences

| SEQ | | | | |
|---|---|---|---|---|
| ID | | | | Motif |
| NO: | Name | Name description | Motif Sequence | length |
| 1 | hp_1 | hairpin 1 | CGCGTCTCTACGTGGGGGC | 22 |
| | | | CCG | |
| 2 | hp_1 | hairpin 1 | CGCGTCTCTACGTGGGGGC | 22 |
| | | | GCG | |
| 3 | hp_3 | hairpin 3 | GGCGCGAAAGCGCC | 14 |
| 4 | PLRVPLRV_22 | potato roll leaf virus | GCGGCACCGTCCGCCCAAA | 22 |
| | | pseudoknot | CGG | |
| 5 | hp_5 | hairpin 5 | GCCCGGCGAAAGCCGGGC | 18 |
| 6 | hp_4 | hairpin 4 | GCCCGGCTTCGGCCGGGC | 18 |
| 7 | hp_2 | hairpin 2 | GGCGCTTCGGCGCC | 14 |
| 8 | MMLV-RT | MML Vaptamer sequence | TTACCACGCGCTCTTAACTG | 33 |
| | aptamer | that can recruit MMLV | CTAGCGCCATGGC | |
| | | RT | | |
| 9 | MS2 | MS2 protein binding | ACATGAGGATCACCCATGT | 19 |
| | | sequence. | | |
| 10 | G quad/ | G-quadruplex in VEGF | GGGCGGGCCGGGGGGGG | 18 |
| | G4_VEGF | promoter | | |
| 11 | C quad/ | C-quadruplex in VEGF | CCCCGCCCCGGCCGCCCC | 18 |
| | IM_VEGF | promoter | | |
| 12 | tRNA_PBS_long | MMLV endogenous | GCTCCTCTGATTGACTACCC | 61 |
| | | binding for replication | GTCAGCGGGGGTCTTTTGG | |
| | | | GGGCTCGTCCGGGATCGGG | |
| | | | AGT | |
| 13 | tRNA_PBS | MMLV endogenous | ACTCCCGATCCCGGACGAG | 61 |
| | long_RC | binding for replication | CCCCCAAAAGACCCCCGCT | |
| | | (reverse complement) | GACGGGTAGTCAATCAGAG | |
| | | | GAGC | |
| 14 | tRNA_PBS_short | MMLV endogenous | TGGGGGCTCGTCCGGGAT | 18 |
| | | binding for replication | | |
| 15 | tRNA_PBS | MMLV endogenous | ATCCCGGACGAGCCCCCA | 18 |
| | short_RC | binding for | | |
| | | replication | | |
| | | (reverse complement) | | |

TABLE 7
Exemplary Nucleic Acid Motif Structural Configurations

| | SEQ | Structural |
|---|---|---|
| Moiety Type | ID NO: | Configuration |
| Hairpin (hp_1) | 1 | [symbol] |
| Pseudoknot (PLRV_22) | 4 | [symbol] |
| tRNA sequence (short) | 14 | [symbol] |
| tRNA sequence (long) | 12 | [symbol] |
| Aptamer (MMLV-RT) | 8 | [symbol] |
| Aptamer (MS2) | 4 | [symbol] |
| Quadruplex (G quad/G4_VEGF) | 8774 | [symbol] |
| Quadruplex (C quad/iM_VEGF) | | [symbol] |

### Tag Sequences

In some embodiments, the PEgRNA comprises a tag sequence in addition to the spacer, gRNA core, primer binding site, and editing template. In some embodiments, the tag sequence comprises a region of complementarity to the editing template. In some embodiments, the tag sequence comprises a region of complementarity to the PBS. In some embodiments, the tag sequence comprises a region of complementarity to the editing template and/or the PBS. In some embodiments, the tag sequence comprises a region of complementarity to the editing template and does not have substantial complementarity to the PBS. In some embodiments, the tag sequence comprises a region of complementarity to the editing template and does not have complementarity to the PBS. In some embodiments, the tag sequence and the editing template each comprises a region of complementarity to each other, wherein the 3′ end of the region of complementarity in the editing template is at a position 1, 2, 3, 4, 5, 6, 7, 8, 9, 10, 11, 12, 13, 14, 15, 16 or more bases 5′ of the 3′ half of the editing template. In some embodiments, the region of complementarity in the tag sequence is at a 5′ portion of the tag sequence. In some embodiments, the tag sequence does not have substantial complementarity to the spacer. In some embodiments, the tag does not have complementarity to the spacer. In some embodiments, the tag sequence is 1, 2, 3, 4, 5, 6, 7, 8, 9, 10, 11, 12, 13, 14, 15, 16 nucleotides in length. In some embodiments, the tag sequence is at least 4, at least 6, at least 8 nucleotides in length. Exemplary Tag sequences can be found in U.S. Patent Application 63/283,076.

### Linkers

In some embodiments, the PEgRNA comprises a linker. In some embodiments, the linker is: i) immediately 5′ of the one or more nucleic acid moieties, ii) immediately 5′ of the tag sequence, iii) immediately 3′ of the tag sequence, iv) immediately 3′ of the spacer, v) immediately 5′ of the spacer, vi) immediately 3′ of the gRNA core, or vii) immediately 5′ of the gRNA core. In some embodiments, the linker is 1, 2, 3, 4, 5, 6, 7, 8, 9, 10, 11, 12, 13, 14, 15, 16 nucleotides in length. In some embodiments, the linker is 2 to 12 nucleotides in length. In some embodiments, the linker is 5 to 20 nucleotides in length. In some embodiments, the linker is 3 to 10, 3 to 15, 3 to 20, 3 to 25, 3 to 30, 3 to 35, 3 to 40, or 3 to 50 nucleotides in length. In some embodiments, the linker is 8 nucleotides in length. In some embodiments, the linker does not form a secondary structure. In some embodiments, the linker does not have a region of complementarity to the PBS sequence. In some embodiments, the linker does not have a region of complementarity to the editing template. As used herein, a linker can be any chemical group or molecule linking two molecules/moieties, e.g., the components of the PEgRNA.

### LegRNAs

Also provided herein are legRNAs. In some embodiments, the PEgRNA is a legRNA. As used herein, a “legRNA” is a PEgRNA comprising a spacer, a gRNA core, a PBS, and an editing template (e.g., an RTT sequence), wherein the PBS and the editing template is positioned within the gRNA core. A legRNA disclosed herein may comprise any 3′ moiety or other modification disclosed herein.

In certain embodiments, the legRNAs comprise in 5′ to 3′ order: i) a spacer that comprises a region of complementarity to a search target sequence in target strand of a double stranded target DNA; ii) a 5′ part of a guide RNA (gRNA) core; iii) an editing template that comprises an intended edit compared to the double stranded target DNA; iv) a primer binding site (PBS) that comprises a region of complementarity to a region upstream of a nick site in a non-target strand of the double stranded target DNA; and v) a 3′ part of a gRNA core. In some embodiments, the 5′ part of the gRNA core comprises a direct repeat, a first stem loop, and a 5′ half of a second stem loop. In some embodiments, the 3′ part of the gRNA core comprises a 3′ half of a second stem loop and a third stem loop. In some embodiments, the 5′ part of the gRNA core and the 3′ part of the gRNA core are “split” at between the 30^{th} and the 31^{st}, the 31^{st} and the 32^{nd}, the 32^{nd} and the 33^{rd}, the 33^{rd} and the 34^{th}, the 34^{th} and the 35^{th}, the 35^{th} and the 36^{th}, the 36^{th} and the 37^{th}, the 37^{th} and the 38^{th}, the 38^{th} and the 39^{th}, or the 39^{th} and 40^{th} nucleotides of the full gRNA core sequence, wherein the position numbering of the nucleotides is as set forth in SEQ ID NO: 16. In some embodiments, the 5′ part of the gRNA core and the 3′ part of the gRNA core are “split” at between the 50^{th} and the 51^{st}, the 51^{st} and the 52^{nd}, the 52^{nd} and the 55^{rd}, the 55^{rd} and the 54^{th}, the 54^{th} and the 55^{th}, the 55^{th} and the 56^{th}, the 56^{th} and the 57^{th}, the 57^{th} and the 58^{th}, the 58^{th} and the 59^{th}, or the 59^{th} and 60^{th} nucleotides of the full gRNA core sequence, wherein the position numbering of the nucleotides is as set forth in SEQ ID NO: 16. In some embodiments, the 5′ part of the gRNA core and the 3′ part of the gRNA core are split between the 54^{th} and the 55^{th} nucleotides of the full gRNA core sequence, wherein the position numbering of the nucleotides is as set forth in SEQ ID NO: 16. In some embodiments, the 5′ part of the gRNA core comprises the sequence GTTTAAGAGCTAGAAATAGCAAGTTTAAATAAGGCTAGTCCGTTATCAGCGTGA (SEQ ID NO: 8775). In some embodiments, the 3′ part of the gRNA core comprises the sequence AAACGCGGCACCGAGTCGGTGC (SEQ ID NO: 8776).

Exemplary legRNA are found in U.S. Patent Application 63/283,076.

In some embodiments, the PEgRNA further comprises a tag sequence that comprises a region of complementarity to the PBS and/or the editing template.

The legRNA may comprise a tag sequence, an aptamer, a hairpin, a quadruplex, a tRNA, a pseudoknot, a linker, or any nucleic acid moieties as described herein. In some embodiments, the legRNA comprises a linker. In some embodiments, the linker is: i) immediately 5′ of the one or more nucleic acid moieties, ii) immediately 5′ of the tag sequence, iii) immediately 3′ of the tag sequence, iv) immediately 3′ of the spacer, v) immediately 5′ of the spacer, vi) immediately 3′ of the gRNA core, and/or vii) immediately 5′ of the gRNA core. In some embodiments, the linker is 1, 2, 3, 4, 5, 6, 7, 8, 9, 10, 11, 12, 13, 14, 15, 16 nucleotides in length. In some embodiments, the linker does not form a secondary structure. In some embodiments, the linker does not have a region of complementarity to the PBS sequence. In some embodiments, the linker does not have a region of complementarity to the editing template. As used herein, a linker can be any chemical group or a molecule linking two molecules or moieties, e.g., the components of the legRNA.

Extended gRNA Cores for Split Synthesis

In some embodiments, a PEgRNA comprises a gRNA core that comprises one or more nucleotide insertions compared to a wild type CRISPR guide RNA scaffold sequence, i.e. an extended in length gRNA core.

In some embodiments, the gRNA core comprises insertion of one or more nucleotides in the direct repeat compared to a wild type CRISPR guide RNA scaffold sequence as set forth in SEQ ID NO: 16. In some embodiments, the gRNA core comprises insertion of one or more nucleotides in the second stem loop compared to a wild type CRISPR guide RNA scaffold sequence as set forth in SEQ ID NO: 16.

Components of a PEgRNA, e.g., an extended PEgRNA, may be synthesized by split synthesis, which refers to synthesizing two (or more) portions of a PEgRNA (e.g., a 5′ half of the PEgRNA and a 3′ half of the PEgRNA) separately and ligating the first half to a second half to form a full length PEgRNA. Exemplary gRNA core sequences for split synthesis are shown in U.S. Patent Application 63/283,076.

In certain embodiments, PEgRNAs provided herein comprise: i) a first sequence comprising a spacer that comprises a region of complementarity to a search target sequence in target strand of a double stranded target DNA, and a first half of a gRNA core; and ii) a second sequence comprising a second half of the gRNA core, an editing template that comprises an intended edit compared to the double stranded target DNA; a primer binding site (PBS) that comprises a region of complementarity to a region upstream of a nick site in a non-target strand of the double stranded target DNA; and, wherein the gRNA core comprises a direct repeat, a first stem loop, and a second stem loop.

In certain embodiments, PEgRNAs provided herein comprise i) a first sequence comprising an editing template that comprises an intended edit compared to the double stranded target DNA; a primer binding site (PBS) that comprises a region of complementarity to a region upstream of a nick site in a non-target strand of the double stranded target DNA; a spacer that comprises a region of complementarity to a search target sequence in target strand of a double stranded target DNA; and a first half of a gRNA core; and ii) a second sequence comprising a second half of a gRNA core, wherein the gRNA core comprises a direct repeat, a first stem loop, and a second stem loop.

In some embodiments, the first sequence is on a first RNA molecule and the second sequence is on a second RNA molecule. In some embodiments, the spacer and the first sequence and the second sequence are on the same RNA molecule. In some embodiments, the first half of the gRNA core and the second half of the gRNA core are selected from the paired first half gRNA core sequences and second half gRNA sequences provided in U.S. Patent Application 63/283,076.

It should be appreciated that the first half and second half of the gRNA core may or may not be equal in length. In some embodiments, the first half of the gRNA core is at least five, at least 10, at least 15, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, or at least 75 nucleotides in length. In some embodiments, the second half of the gRNA core is at least five, at least 10, at least 15, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, or at least 75 nucleotides in length.

In some embodiments, the first half of the gRNA core is at least 80%, at least 85%, at least 90%, at least 95%, at least 99% identical to a sequence provided in U.S. Patent Application 63/283,076. In some embodiments, the first half of the gRNA core is identical to a sequence provided in U.S. Patent Application 63/283,076. In some embodiments, the second half of the gRNA core is at least 80%, at least 85%, at least 90%, at least 95%, at least 99% identical to a sequence provided in U.S. Patent Application 63/283,076. In some embodiments, the second half of the gRNA core is identical to a sequence provided in U.S. Patent Application 63/283,076.

As previously discussed, the gRNA core may comprise a direct repeat and/or one or multiple stem loops. In some embodiments, gRNA cores synthesize using split synthesis comprise a first half of a gRNA core comprising a first half of the direct repeat and a second half of a gRNA core comprising the second half of the direct repeat. In some embodiments, gRNA cores synthesizes using split synthesis comprises a first half of a gRNA core comprising a first half of the second stem loop and a second half of a gRNA core comprising the second half of the second stem loop.

### Nucleotide Editing

Provided herein are exemplary PEgRNAs with modifications disclosed herein for nucleotide editing. An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the gene to be edited. Position of the intended nucleotide edit(s) relevant to other components of the PEgRNA, or to particular nucleotides (e.g., mutations) in the target gene may vary. In some embodiments, the nucleotide edit is in a region of the PERNA corresponding to or homologous to the protospacer sequence. In some embodiments, the nucleotide edit is in a region of the PEgRNA corresponding to a region of the gene outside of the protospacer sequence.

In some embodiments, the position of a nucleotide edit incorporation in the target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the target gene, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream of the 5′ most nucleotide of the PAM sequence in the edit strand of the target gene. By 0 base pair upstream or downstream of a reference position, it is meant that the intended nucleotide is immediately upstream or downstream of the reference position. In some embodiments, a nucleotide edit is incorporated at a position corresponding to about 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream of the 5′ most nucleotide of the PAM sequence. In some embodiments, the nucleotide edit is incorporated at a position corresponding to 3 base pairs upstream of the 5′ most nucleotide of the PAM sequence. In some embodiments, the nucleotide edit in is incorporated at a position corresponding to 4 base pairs upstream of the 5′ most nucleotide of the PAM sequence. In some embodiments, the nucleotide edit is incorporated at a position corresponding to 5 base pairs upstream of the 5′ most nucleotide of the PAM sequence. In some embodiments, the nucleotide edit in the editing template is at a position corresponding to 6 base pairs upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs downstream of the 5′ most nucleotide of the PAM sequence in the edit strand of the target gene. In some embodiments, a nucleotide edit is incorporated at a position corresponding to about 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs downstream of the 5′ most nucleotide of the PAM sequence. In some embodiments, a nucleotide edit is incorporated at a position corresponding to 3 base pairs downstream of the 5′ most nucleotide of the PAM sequence. In some embodiments, a nucleotide edit is incorporated at a position corresponding to 4 base pairs downstream of the 5′ most nucleotide of the PAM sequence. In some embodiments, a nucleotide edit is incorporated at a position corresponding to 5 base pairs downstream of the 5′ most nucleotide of the PAM sequence. In some embodiments, a nucleotide edit is incorporated at a position corresponding to 6 base pairs downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

When referred to in the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream to the 5′ most nucleotide of the PBS. In some embodiments, the intended nucleotide edit is 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream to the 5′ most nucleotide of the PBS.

The corresponding positions of the intended nucleotide edit incorporated in the target gene may also be referred to bases on the nicking position generated by a split prime editor based on sequence homology and complementarity. For example, in embodiments, the distance between the nucleotide edit to be incorporated into the target gene and the nick generated by the split prime editor may be determined when the spacer hybridizes with the search target sequence and the extension arm hybridizes with the editing target sequence. In certain embodiments, the position of the nucleotide edit can be in any position downstream of the nick site on the edit strand (or the PAM strand) generated by the split prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pairs from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

A PEgRNA may also comprise optional modifiers, e.g., 3′ end modifier region and/or a 5′ end modifier region. In some embodiments, a PERNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PERNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PERNA comprises a toeloop sequence at the 3′ end. In some embodiments, the PEgRNA comprises a 3′ extension arm and a toeloop sequence at the 3′ end of the extension arm. In some embodiments, the PERNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a prime editing system or composition further comprises a nick guide polynucleotide, such as a nick guide RNA (ngRNA). Without wishing to be bound by any particular theory, the non-edit strand of a double stranded target DNA in the target gene may be nicked by a CRISPR-Cas nickase directed by an ngRNA. In some embodiments, the nick on the non-edit strand directs endogenous DNA repair machinery to use the edit strand as a template for repair of the non-edit strand, which may increase efficiency of prime editing. In some embodiments, the non-edit strand is nicked by a split prime editor localized to the non-edit strand by the ngRNA. Accordingly, also provided herein are PERNA systems comprising at least one PERNA and at least one ngRNA.

In some embodiments, the ngRNA is a guide RNA which contains a variable spacer sequence and a guide RNA scaffold or core region that interacts with the DNA binding domain, e.g., Cas9 of the split prime editor. In some embodiments, the ngRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the ng search target sequence recognized by the ng spacer and the search target sequence recognized by the spacer sequence of the PEgRNA are on opposite strands of the double stranded target DNA of target gene, e.g., the gene. A prime editing system or complex comprising a ngRNA may be referred to as a “PE3” prime editing system or PE3 prime editing complex.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. Such a prime editing system may be referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

A PERNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs and/or ngRNAs provided in this disclosure may have undergone a chemical or biological modifications. Modifications may be made at any position within a PERNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, a chemical modification may be within the 5′ most end of a PEgRNA or ngRNA. In some embodiments, a PERNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PERNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PERNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a 2′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Pharmaceutical Compositions

Disclosed herein are pharmaceutical compositions comprising any of the prime editing composition components, for example, split prime editors, fusion proteins, polynucleotides encoding split prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, with a PERNA and a split prime editor described herein. In some embodiments, the target gene is double stranded, and comprises two strands of DNA complementary to each other. In some embodiments, the contacting with a PEgRNA and the contacting with a split prime editor are performed sequentially. In some embodiments, the contacting with a split prime editor is performed after the contacting with a PERNA. In some embodiments, the contacting with a PEgRNA is performed after the contacting with a split prime editor. In some embodiments, the contacting with a PEgRNA, and the contacting with a split prime editor are performed simultaneously. In some embodiments, the PEgRNA and the split prime editor are associated in a complex prior to contacting a target gene.

In some embodiments, contacting the target gene with the prime editing composition results in binding of the PEgRNA to a target strand of the target gene. In some embodiments, contacting the target gene with the prime editing composition results in binding of the PERNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the split prime editor to the target gene, e.g., the target gene, upon the contacting of the PE composition with the target gene. In some embodiments, the DNA binding domain of the PE associates with the PEgRNA. In some embodiments, the PE binds the target gene, directed by the PERNA. Accordingly, in some embodiments, the contacting of the target gene result in binding of a DNA binding domain of a split prime editor of the target gene directed by the PERNA.

In some embodiments, contacting the target gene with the prime editing composition results in a nick in an edit strand of the target gene, by the split prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the split prime editor, thereby generating a single-stranded DNA comprising a free 3′ end at the nick site. In some embodiments, the DNA binding domain of the split prime editor is a Cas domain. In some embodiments, the DNA binding domain of the split prime editor is a Cas9. In some embodiments, the DNA binding domain of the split prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the split prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the split prime editor. In some embodiments, the method comprises contacting the target gene with a DNA polymerase, e.g., a reverse transcriptase, as a part of a split prime editor protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a split prime editor protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the split prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the split prime editor.

In some embodiments, the target gene, is in a cell. Accordingly, also provided herein are methods of modifying a cell, such as a human cell, a human primary cell, and/or a human iPSC-derived cell.

In some embodiments, the prime editing method comprises introducing a PEgRNA, a split prime editor, and/or a ngRNA into the cell that has the target gene. In some embodiments, the prime editing method comprises introducing into the cell that has the target gene with a prime editing composition comprising a PERNA, a split prime editor polypeptide, and/or a ngRNA. In some embodiments, the PEgRNA, the split prime editor polypeptide, and/or the ngRNA form a complex prior to the introduction into the cell. In some embodiments, the PEgRNA, the split prime editor polypeptide, and/or the ngRNA form a complex after the introduction into the cell. The split prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The split prime editors, PEgRNA and/or ngRNAs, and prime editing complexes may be introduced into the cell simultaneously or sequentially.

In some aspects, the disclosure provides a lipid nanoparticle or ribonucleoprotein comprising the prime editing system, or a component thereof, herein described. In certain aspects, the disclosure provides a polynucleotide encoding the prime editor herein described. In certain aspects, the disclosure provides a polynucleotide encoding the first polypeptide herein described. In certain aspects, the disclosure provides a polynucleotide encoding the second polypeptide herein described.

In some embodiments, the prime editing method comprises introducing into the cell a PEgRNA or a polynucleotide encoding the PEgRNA, a split prime editor polynucleotide encoding a split prime editor polypeptide, and optionally an ngRNA or a polynucleotide encoding the ngRNA. In some embodiments, the method comprises introducing the PERNA or the polynucleotide encoding the PEgRNA, the polynucleotide encoding the split prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the split prime editor polypeptide, and/or the ngRNA or the polynucleotide encoding the ngRNA into the cell sequentially. In some embodiments, the method comprises introducing the polynucleotide encoding the split prime editor polypeptide into the cell before introduction of the PEgRNA or the polynucleotide encoding the PEgRNA and/or the ngRNA or the polynucleotide encoding the ngRNA. In some embodiments, the polynucleotide encoding the split prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the split prime editor polypeptide is introduced into the cell after the PEgRNA or the polynucleotide encoding the PEgRNA and/or the ngRNA or the polynucleotide encoding the ngRNA are introduced into the cell. The polynucleotide encoding the split prime editor polypeptide, the PEgRNA or the polynucleotide encoding the PERNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the split prime editor polypeptide, the polynucleotide encoding the PEgRNA, and/or the polynucleotide encoding the ngRNA integrate into the genome of the cell after being introduced into the cell. In some embodiments, the polynucleotide encoding the split prime editor polypeptide, the polynucleotide encoding the PERNA, and/or the polynucleotide encoding the ngRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a human cell from an organ. In some embodiments, the cell is a primary human cell de

In some embodiments, the cell is a progenitor cell. In some embodiments, the cell is a stem cell. in some embodiments, the cell is an induced pluripotent stem cell. In some embodiments, the cell is an embryonic stem cell. In some embodiments, the cell is a retinal progenitor cell. In some embodiments, the cell is a retina precursor cell. In some embodiments, the cell is a fibroblast.

In some embodiments, the cell is a human stem cell. in some embodiments, the cell is an induced human pluripotent stem cell. In some embodiments, the cell is a human embryonic stem cell. In some embodiments, the cell is a human retinal progenitor cell. In some embodiments, the cell is a human retina precursor cell. In some embodiments, the cell is a human fibroblast.

In some embodiments, the cell is a primary cell. In some embodiments, the cell is a human primary cell. In some embodiments, the cell is a retina cell. In some embodiments, the cell is a photoreceptor. In some embodiments, the cell is a rod cell. In some embodiments, the cell is a cone cell. In some embodiments, the cell is a human cell from a retina. In some embodiments, the cell is a human photoreceptor. In some embodiments, the cell is a human rod cell. In some embodiments, the cell is a human cone cell. In some embodiments, the cell is a primary human photoreceptor derived from an induced human pluripotent stem cell (iPSC).

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the split prime editor polypeptide or the polynucleotide encoding the split prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the split prime editor polypeptide or the polynucleotide encoding the split prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the split prime editor polypeptide or the polynucleotide encoding the split prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the split prime editor polypeptide or the polynucleotide encoding the split prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the split prime editor polypeptide or the polynucleotide encoding the split prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. In some embodiments, a prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a hepatocyte relative to a corresponding control hepatocyte. In some embodiments, the hepatocyte is a human hepatocyte.

In some embodiments, the prime editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37 (3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a gene within the genome of a cell) to a prime editing composition.

In some embodiments, the prime editing compositions provided herein are capable of incorporated one or more intended nucleotide edits efficiently without generating a significant proportion of indels. In some embodiments, the prime editing methods disclosed herein have an editing efficiency of at least about 1% and an indel frequency of less than 1% in a target cell, e.g., a human primary cell, human iPSC, or human fibroblast. In some embodiments, the prime editing methods disclosed herein have an editing efficiency of at least about 1% and an indel frequency of less than 0.5% in a target cell, e.g., a human primary cell, human iPSC, or human fibroblast. In some embodiments, the prime editing methods disclosed herein have an editing efficiency of at least about 1% and an indel frequency of less than 0.1% in a target cell, e.g., a human primary cell, a human iPSC, or a human fibroblast. In some embodiments, the prime editing methods disclosed herein have an editing efficiency of at least about 5% and an indel frequency of less than 1% in a target cell, e.g., a human primary cell, a human iPSC, or a human fibroblast. In some embodiments, the prime editing methods disclosed herein have an editing efficiency of at least about 5% and an indel frequency of less than 0.5% in a target cell, e.g., a human primary cell, a human iPSC, or a human fibroblast. In some embodiments, the prime editing methods disclosed herein have an editing efficiency of at least about 5% and an indel frequency of less than 0.1% in a target cell, e.g., a human primary cell, a human iPSC, or a human fibroblast.

In some embodiments, the prime editing methods disclosed herein have an editing efficiency of at least about 7.5% and an indel frequency of less than 1% in a target cell, e.g., a human primary cell, a human iPSC, or a human fibroblast. In some embodiments, the prime editing methods disclosed herein have an editing efficiency of at least about 7.5% and an indel frequency of less than 0.5% in a target cell, e.g., a human primary cell, a human iPSC, or a human fibroblast. In some embodiments, the prime editing methods disclosed herein have an editing efficiency of at least about 7.5% and an indel frequency of less than 0.1% in a target cell, e.g., a human primary cell, a human iPSC, or a human fibroblast.

In some embodiments, the prime editing methods disclosed herein have an editing efficiency of at least about 10% and an indel frequency of less than 1% in a target cell, e.g., a human primary cell, a human iPSC, or a human fibroblast. In some embodiments, the prime editing methods disclosed herein have an editing efficiency of at least about 10% and an indel frequency of less than 0.5% in a target cell, e.g., a human primary cell, a human iPSC, or a human fibroblast. In some embodiments, the prime editing methods disclosed herein have an editing efficiency of at least about 10% and an indel frequency of less than 0.1% in a target cell, e.g., a human primary cell, a human iPSC, or a human fibroblast.

In some embodiments, the prime editing methods disclosed herein have an editing efficiency of at least about 15% and an indel frequency of less than 1% in a target cell, e.g., a human primary cell, a human iPSC, or a human fibroblast. In some embodiments, the prime editing methods disclosed herein have an editing efficiency of at least about 15% and an indel frequency of less than 0.5% in a target cell, e.g., a human primary cell, a human iPSC, or a human fibroblast. In some embodiments, the prime editing methods disclosed herein have an editing efficiency of at least about 15% and an indel frequency of less than 0.1% in a target cell, e.g., a human primary cell, a human iPSC, or a human fibroblast.

In some embodiments, the prime editing methods disclosed herein have an editing efficiency of at least about 20% and an indel frequency of less than 1% in a target cell, e.g., a human primary cell, a human iPSC, or a human fibroblast. In some embodiments, the prime editing methods disclosed herein have an editing efficiency of at least about 20% and an indel frequency of less than 0.5% in a target cell, e.g., a human primary cell, a human iPSC, or a human fibroblast. In some embodiments, the prime editing methods disclosed herein have an editing efficiency of at least about 20% and an indel frequency of less than 0.1% in a target cell, e.g., a human primary cell, a human iPSC, or a human fibroblast.

In some embodiments, the prime editing methods disclosed herein have an editing efficiency of at least about 30% and an indel frequency of less than 1% in a target cell, e.g., a human primary cell, a human iPSC, or a human fibroblast. In some embodiments, the prime editing methods disclosed herein have an editing efficiency of at least about 30% and an indel frequency of less than 0.5% in a target cell, e.g., a human primary cell, a human iPSC, or a human fibroblast. In some embodiments, the prime editing methods disclosed herein have an editing efficiency of at least about 30% and an indel frequency of less than 0.1% in a target cell, e.g., a human primary cell, a human iPSC, or a human fibroblast.

In some embodiments, the prime editing methods disclosed herein have an editing efficiency of at least about 40% and an indel frequency of less than 1% in a target cell, e.g., a human primary cell, a human iPSC, or a human fibroblast. In some embodiments, the prime editing methods disclosed herein have an editing efficiency of at least about 40% and an indel frequency of less than 0.5% in a target cell, e.g., a human primary cell, a human iPSC, or a human fibroblast. In some embodiments, the prime editing methods disclosed herein have an editing efficiency of at least about 40% and an indel frequency of less than 0.1% in a target cell, e.g., a human primary cell, a human iPSC, or a human fibroblast.

In some embodiments, the prime editing methods disclosed herein have an editing efficiency of at least about 50% and an indel frequency of less than 1% in a target cell, e.g., a human primary cell, a human iPSC, or a human fibroblast. In some embodiments, the prime editing methods disclosed herein have an editing efficiency of at least about 50% and an indel frequency of less than 0.5% in a target cell, e.g., a human primary cell, a human iPSC, or a human fibroblast. In some embodiments, the prime editing methods disclosed herein have an editing efficiency of at least about 50% and an indel frequency of less than 0.1% in a target cell, e.g., a human primary cell, a human iPSC, or a human fibroblast.

In some embodiments, the prime editing methods disclosed herein have an editing efficiency of at least about 60% and an indel frequency of less than 1% in a target cell, e.g., a human primary cell, a human iPSC, or a human fibroblast. In some embodiments, the prime editing methods disclosed herein have an editing efficiency of at least about 60% and an indel frequency of less than 0.5% in a target cell, e.g., a human primary cell, a human iPSC, or a human fibroblast. In some embodiments, the prime editing methods disclosed herein have an editing efficiency of at least about 60% and an indel frequency of less than 0.1% in a target cell, e.g., a human primary cell, a human iPSC, or a human fibroblast.

In some embodiments, the prime editing methods disclosed herein have an editing efficiency of at least about 70% and an indel frequency of less than 1% in a target cell, e.g., a human primary cell, a human iPSC, or a human fibroblast. In some embodiments, the prime editing methods disclosed herein have an editing efficiency of at least about 70% and an indel frequency of less than 0.5% in a target cell, e.g., a human primary cell, a human iPSC, or a human fibroblast. In some embodiments, the prime editing methods disclosed herein have an editing efficiency of at least about 70% and an indel frequency of less than 0.1% in a target cell, e.g., a human primary cell, a human iPSC, or a human fibroblast.

In some embodiments, the prime editing methods disclosed herein have an editing efficiency of at least about 80% and an indel frequency of less than 1% in a target cell, e.g., a human primary cell, a human iPSC, or a human fibroblast. In some embodiments, the prime editing methods disclosed herein have an editing efficiency of at least about 80% and an indel frequency of less than 0.5% in a target cell, e.g., a human primary cell, a human iPSC, or a human fibroblast. In some embodiments, the prime editing methods disclosed herein have an editing efficiency of at least about 80% and an indel frequency of less than 0.1% in a target cell, e.g., a human primary cell, a human iPSC, or a human fibroblast.

In some embodiments, the prime editing methods disclosed herein have an editing efficiency of at least about 90% and an indel frequency of less than 1% in a target cell, e.g., a human primary cell, a human iPSC, or a human fibroblast. In some embodiments, the prime editing methods disclosed herein have an editing efficiency of at least about 90% and an indel frequency of less than 0.5% in a target cell, e.g., a human primary cell, a human iPSC, or a human fibroblast. In some embodiments, the prime editing methods disclosed herein have an editing efficiency of at least about 90% and an indel frequency of less than 0.1% in a target cell, e.g., a human primary cell, a human iPSC, or a human fibroblast.

In some embodiments, the prime editing methods disclosed herein have an editing efficiency of at least about 95% and an indel frequency of less than 1% in a target cell, e.g., a human primary cell, a human iPSC, or a human fibroblast. In some embodiments, the prime editing methods disclosed herein have an editing efficiency of at least about 95% and an indel frequency of less than 0.5% in a target cell, e.g., a human primary cell, a human iPSC, or a human fibroblast. In some embodiments, the prime editing methods disclosed herein have an editing efficiency of at least about 95% and an indel frequency of less than 0.1% in a target cell, e.g., a human primary cell, a human iPSC, or a human fibroblast. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a gene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., PEgRNAs and split prime editors as described herein) and prime editing methods disclosed herein can be used to edit a target gene. In some embodiments, the target gene comprises a mutation compared to a wild type gene. In some embodiments, the mutation is associated a disease. In some embodiments, the target gene comprises an editing target sequence that contains the mutation associated with a disease. In some embodiments, the mutation is in a coding region of the target gene. In some embodiments, the mutation is in an exon of the target gene. In some embodiments, the prime editing method comprises contacting a target gene with a prime editing composition comprising a split prime editor, a PERNA, and/or a ngRNA. In some embodiments, contacting the target gene with the prime editing composition results in incorporation of one or more intended nucleotide edits in the target gene. In some embodiments, the incorporation is in a region of the target gene that corresponds to an editing target sequence in the gene. In some embodiments, the one or more intended nucleotide edits comprises a single nucleotide substitution, an insertion, a deletion, or any combination thereof, compared to the endogenous sequence of the target gene. In some embodiments, incorporation of the one or more intended nucleotide edits results in replacement of one or more mutations with the corresponding sequence that encodes a wild type protein. In some embodiments, incorporation of the one or more intended nucleotide edits results in replacement of the one or more mutations with the corresponding sequence in a wild type gene. In some embodiments, incorporation of the one more intended nucleotide edits results in correction of a mutation in the target gene. In some embodiments, the target gene comprises an editing template sequence that contains the mutation. In some embodiments, contacting the target gene with the prime editing composition results in incorporation of one or more intended nucleotide edits in the target gene, which corrects the mutation in the editing target sequence (or a double stranded region comprising the editing target sequence and the complementary sequence to the editing target sequence on a target strand) in the target gene.

In some embodiments, incorporation of the one more intended nucleotide edits results in correction of a mutation in the target gene. In some embodiments, incorporation of the one more intended nucleotide edits results in correction of a gene sequence and restores wild type expression and function of the protein.

In some embodiments, the target gene is in a target cell. Accordingly, in one aspect provided herein is a method of editing a target cell comprising a target gene that encodes a polypeptide that comprises one or more mutations relative to a wild type gene. In some embodiments, the methods of the present disclosure comprise introducing a prime editing composition comprising a PEgRNA, a split prime editor polypeptide, and/or a ngRNA into the target cell that has the target gene to edit the target gene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the target cell is a hepatocyte. In some embodiments, the target cell is a human hepatocyte. In some embodiments, the target cell is a primary human hepatocyte derived from an induced human pluripotent stem cell (iPSC). In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target gene that comprises one or more mutations restores wild type expression and function of protein encoded by the gene. In some embodiments, the target gene encodes at least one mutation as compared to the wild type protein prior to incorporation of the one or more intended nucleotide edits. In some embodiments, expression and/or function of protein may be measured when expressed in a target cell. In some embodiments, incorporation of the one or more intended nucleotide edits in the target gene comprising one or more mutations lead to a fold change in a level of gene expression, protein expression, or a combination thereof. In some embodiments, a change in the level of gene expression can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target gene that comprises one or more mutations restores wild type expression of protein by at least 10%, 20%, 30%, 40%, 50%, 60%, 70%, 80%, 90%, 95%, 099% or more as compared to wild type expression of the protein in a suitable control cell that comprises a wild type gene.

In some embodiments, an expression increase can be measured by a functional assay. In some embodiments, protein expression can be measured using a protein assay. In some embodiments, protein expression can be measured using antibody testing. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel.

### Delivery

Prime editing compositions described herein can be delivered to a cellular environment with any approach known in the art. Components of a prime editing composition can be delivered to a cell by the same mode or different modes. For example, in some embodiments, a split prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a split prime editor polypeptide, a PEgRNA and/or a ngRNA is encoded by a polynucleotide. In some embodiments, the polynucleotide encodes a split prime editor protein comprising a DNA binding domain and a DNA polymerase domain. In some embodiments, the polynucleotide encodes a DNA polymerase domain of a split prime editor. In some embodiments, the polynucleotide encodes a DNA polymerase domain of a split prime editor. In some embodiments, the polynucleotide encodes a portion of a split prime editor protein, for example, a N-terminal portion of a split prime editor protein connected to an intein-N. In some embodiments, the polynucleotide encodes a portion of a split prime editor protein, for example, a C-terminal portion of a split prime editor protein connected to an intein-C. In some embodiments, the polynucleotide encodes a PEgRNA and/or a ngRNA. In some embodiments, the polypeptide encodes two or more components of a prime editing composition, for example, a split prime editor protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a split prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the split prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target gene or can be introduced into a cell using any suitable technique for introducing nucleic acids into cells (e.g., microinjection, electroporation, transfection). In some embodiments, the split prime editor-coding sequences, the PEgRNAs, and/or the ngRNAs are modified to include one or more modified nucleoside e.g., using pseudo-U or 5-Methyl-C.

Methods of non-viral delivery of nucleic acids can include lipofection, nucleofection, microinjection, biolistics, virosomes, liposomes, immunoliposomes, polycation or lipid: nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid: nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector (e.g., a trans-splicing AAV vector). In some embodiments, an AAV viral vector may be used for trans-splicing system to express components of split prime editors (e.g., express components of split prime editors separately and/or spliced together).

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and w2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome.

In some embodiments, dual AAV vectors are generated by splitting a large transgene expression cassette in two separate halves (5′ and 3′ ends that encode N-terminal portion and C-terminal portion of, e.g., a split prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a split prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a split prime editor protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, a polynucleotide encoding a split prime editor protein is split in two separate halves, each encoding a portion of the split prime editor protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length split prime editor protein is reassembled upon co-infection of the same cell by both dual AAV vectors, expression of both halves of the split prime editor protein, and self-excision of the inteins.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a split prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a split prime editor protein can be provided to cells as a polypeptide. In some embodiments, the split prime editor protein is fused to a polypeptide domain that increases solubility of the protein. In some embodiments, the split prime editor protein is formulated to improve solubility of the protein.

In some embodiment, a split prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 8777). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine (SEQ ID NO: 8778), and octa-arginine (SEQ ID NO: 8779). The nona-arginine (R9) sequence (SEQ ID NO: 8778) can be used. The site at which the fusion can be made may be selected in order to optimize the biological activity, secretion or binding characteristics of the polypeptide.

In some embodiments, a split prime editor polypeptide is produced in vitro or by host cells, and it may be further processed by unfolding, e.g., heat denaturation, DTT reduction, etc. and may be further refolded. In some embodiments, a split prime editor polypeptide is prepared by in vitro synthesis. Various commercial synthetic apparatuses can be used. By using synthesizers, naturally occurring amino acids can be substituted with unnatural amino acids. In some embodiments, a split prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, split prime editor polypeptide components and PERNA/ngRNA are introduced to a target cell by nanoparticles. In some embodiments, the split prime editor polypeptide components and the PERNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a hepatocyte, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 8 below.

In some embodiments, components of a prime editing composition form a complex prior to delivery to a target cell. For example, a split prime editor protein, a PEgRNA, and/or a ngRNA can form a complex prior to delivery to the target cell. In some embodiments, a prime editing polypeptide (e.g., a split prime editor protein) and a guide polynucleotide (e.g., a PEgRNA or ngRNA) form a ribonucleoprotein (RNP) for delivery to a target cell. In some embodiments, the RNP comprises a split prime editor protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 8 below.

TABLE 8
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N- | DOTMA | Cationic |
| trimethylammonium chloride | | |
| 1,2-Dioleoyloxy-3-trimethylammonium-propane | DOGS | Cationic |
| Dioctadecylamidoglycylspermine | | |
| N-(3-Aminopropy1)-N,N-dimethy1-2,3-bis(dodecyloxy)- | GAP-DLRIE | Cationic |
| 1-propanaminium bromide | | |
| Cetyltrimethylammonium bromide | CTAB | Cationic |
| 6-Lauroxyhexyl omithinate | LHON | Cationic |
| 1-(2,3-Dioleoyloxypropy1)-2,4,6-trimethylpyridinium | 2Oc | Cationic |
| 2,3-Dioleyloxy-N-P(spenninecarboxamido-ethy1J- | DOSPA | Cationic |
| N,Ndimethyl-1-propanatninium trifluoroacetate | | |
| 1,2-Dioley 1-3-trimethylamtnonium-propane | DOPA | Cationic |
| N-(2-Hydroxyethyl)-N,N-dimethy1-2,3- | MDRIE | Cationic |
| bis(tetradecyloxy)-1-propanaminium bromide | | |
| Dimyristooxypropyl dimethyl hydroxyethyl ammonium | DMRI | Cationic |
| bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecyl-3- | diC14- | Cationic |
| tetradecylaminopropionamidine | amidine | |
| Octadecenolyoxy[ethyl-2-heptadeceny1-3 | DOTIM | Cationic |
| hydroxyethyl] imidazolinium chloride | | |
| N1-Cholesteryloxycarbonyl-3,7-diazanonane-1,9- | CDAN | Cationic |
| diamine | | |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 9 below.

TABLE 9
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 10 below.

TABLE 10
Exemplary polynucleotide delivery methods

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic |
| | electroporation, | | | | Acids and |
| | particle gun, | | | | Proteins |
| | Calcium | | | | |
| | phosphate | | | | |
| | transfection) | | | | |
| Viral | Retrovirus | NO | Stable | YES | RNA |
| | Lentivirus | YES | Stable | YES/NO | RNA |
| | | | | with | |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes | YES | Stable | NO | DNA |
| | Simplex Virus | | | | |
| Non-Viral | Cationic | YES | Transient | Depends | Nucleic |
| | | | | on what is | acids and |
| | | | | delivered | Proteins |
| | Polymeric | YES | Transient | NO | Nucleic |
| | Nanoparticles | | | | Acids |
| Biological | Attenuated | YES | Transient | NO | Nucleic |
| | Bacteria | | | | Acids |
| Non-Viral | Engineered | YES | Transient | NO | Nucleic |
| Delivery | Bacteriophages | | | | Acids |
| Vehicles | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prim editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prime editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### Kits

In certain aspects, the disclosure provides a kit comprising a first polynucleotide and a second polynucleotide. In some embodiments, the first polynucleotide is any polynucleotide herein described and the second polynucleotide is any polynucleotide herein described. In some embodiments, the first and/or second polynucleotide is in any vector as herein described.

In some embodiments, the vector is an AAV vector.

### EXAMPLES

### Example 1: Split Editor System with NANOBODY®

Protein fusions via a peptide linker have been shown to have many benefits, including improved stability and increased activity via increasing the local concentration of the components involved in the systems. However, protein linkers can impede activity by forcing unfavorable steric interactions between the protein components and substrates. Unfavorable steric conditions may especially apply to prime editing, where many coordinated actions must occur for successful activity, including multiple conformational changes and substrate turnover. To investigate this possibility, Applicant developed a split prime editing system in which the covalent protein linker in an exemplary prime editor fusion protein (PE2) was replaced with a NANOBODY® peptide system.

The split prime editing systems were designed to include a portion of the prime editing system fused to a NANOBODY® and a second portion of the prime editing system fused to a target peptide.

The exemplary split prime editing systems include i) a Cas9 component fused to either to a NANOBODY® or a target peptide and ii) a Moloney Murine Leukemia Virus (MMLV) reverse transcriptase (RT) fused to the corresponding target peptide or NANOBODY®.

To test if the orientation mattered, the NANOBODY® was fused to either the Cas9 portion or the RT portion of the prime editing system and vice-versa (as shown in FIGS. 1-4).

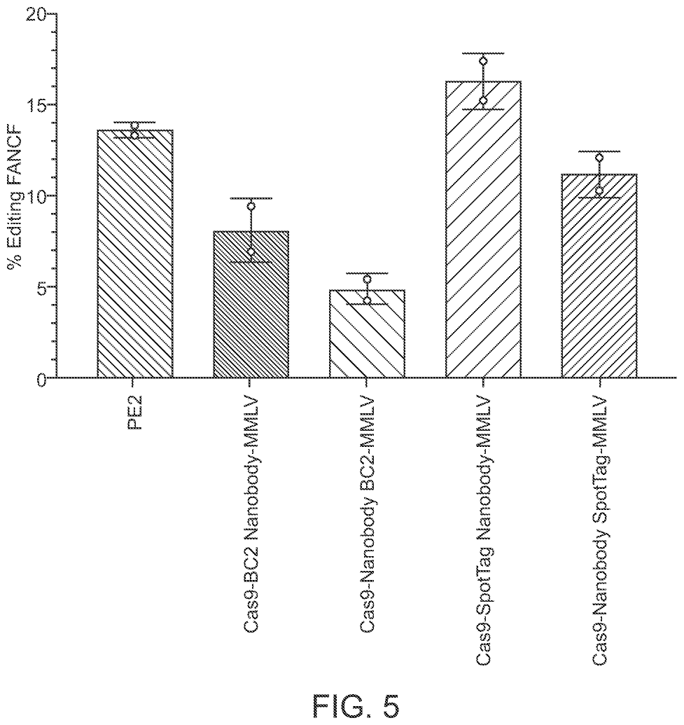

The activity of split prime editing systems was tested in mammalian cells. In particular, four different constructs (as shown in FIGS. 1-4) were tested for editing activity of a target gene site. In this example the target gene site was the Fanconi anemia complementation group F (FANCF) gene site in HEK293 cells. The split prime editing system was introduced to the HEK293 cells via a plasmid that expressed a single protein in which the Cas9+Nanobody/peptide and MMLV+peptide/Nanobody polypeptides were fused via a self-cleaving peptide linker. Following expression in the HEK293 cells, cleavage of the self-cleaving peptide linker results in two separate polypeptides, mimicking trans delivery of the split prime editor. The split prime editing NANOBODY® system was observed to efficiently edit the target gene (as shown in FIG. 5).

### INCORPORATION BY REFERENCE

All publications and patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the methods and compositions provided herein. Such equivalents are intended to be encompassed by the following claims.

What is claimed is:

1. A split prime editing system:
A) a first polypeptide, or a polynucleotide encoding the first polypeptide, the first polypeptide comprising a DNA binding domain fused to a first affinity moiety selected from:
i) a single-domain antibody sequence, or
ii) a peptide tag; and
B) a second polypeptide, or a polynucleotide encoding the second polynucleotide, the second polynucleotide comprising a DNA polymerase domain fused to a second affinity moiety that is:
i) the peptide tag if the DNA binding domain is fused to the single-domain antibody sequence, or
ii) the single-domain antibody sequence if the DNA binding domain is fused to the peptide tag;
wherein the peptide tag is an antigen for which the single-domain antibody sequence has sufficient affinity to bind under physiological conditions.

2. The system of claim 1, wherein the DNA binding domain comprises an HNH domain and/or a RuvC domain.

3. The system of claim 2, wherein the DNA binding domain comprises both an HNH domain and a RuvC domain.

4. The system of claim 3, wherein the DNA binding protein comprises a mutation that decreases or eliminates nuclease activity in the RuvC domain.

5. The system of claim 1, wherein the DNA binding domain is a Type II Cas protein.

6. The system of claim 5, wherein the Type II Cas protein is a Cas9 protein.

7. The system of claim 6, wherein the Cas9 protein is a Cas9 nickase.

8. The system of claim 1, wherein the DNA binding domain is a Type V Cas protein.

9. The system of claim 1, wherein the DNA binding domain is a Cas12 protein.

10. The system of claim 1, wherein the DNA binding domain has a sequence with at least 80%, 85%, 90%, 95%, 96%, 97%, 98%, or 99% identity to a sequence from Table 14.

11. The system of claim 1, wherein the DNA binding domain has a sequence from Table 14.

12. The system of any one of claims 10-11, wherein the sequence from Table 14 is SEQ ID NO: 8000.

13. The system of any one of claims 1-12, wherein the DNA polymerase domain is a reverse transcriptase domain.

14. The system of claim 13, wherein the reverse transcriptase domain is a Maloney Murine Leukemia Virus (MMLV) reverse transcriptase.

15. The system of any one of claims 1-12, wherein the DNA polymerase domain comprises a sequence with at least 80%, 85%, 90%, 95%, 96%, 97%, 98%, or 99% identity to a sequence from Table 11, Table 12, or Table 13.

16. The system of any one of claims 1-12, wherein the DNA polymerase domain comprises a sequence from Table 11, Table 12, or Table 13.

17. The system of any one of claims 1-14, wherein the DNA polymerase domain comprises a sequence having at least 80%, 85%, 90%, 95%, 96%, 97%, 98%, 99%, or 100% identity to SEQ ID NO: 4448 or SEQ ID NO: 8001.

18. The system of any one of claims 1-17, wherein the single-domain antibody sequence has at least 80%, 85%, 90%, 95%, 96%, 97%, 98%, or 99% identity to SEQ ID NO: 8002.

19. The system of any one of claims 1-17, wherein the single-domain antibody sequence is SEQ ID NO: 8002.

20. The system of any one of claims 1-19, wherein the peptide tag has a sequence from Table 16 or a sequence with 1 or 2 substitutions relative to a sequence from Table 16.

21. The system of any one of claims 1-19, wherein the peptide tag has a sequence from Table 16.

22. The system of any one of claims 1-19, wherein the peptide tag is SEQ ID NO: 8003.

23. The system of any one of claims 1-22, wherein the DNA binding domain is located N-terminally to the first affinity moiety.

24. The system of any one of claims 1-23, further comprising a first peptide linker between the DNA binding domain and the first affinity moiety.

25. The system of claim 24, wherein the first peptide linker comprises a sequence from Table 15.

26. The system of any one of claims 1-25, wherein the DNA polymerase domain is located C-terminally to the second affinity moiety.

27. The system of any one of claims 1-26, further comprising a second peptide linker between the DNA polymerase domain and the second affinity moiety.

28. The system of claim 27, wherein the second peptide linker comprises a sequence from Table 15.

29. The system of any one of claims 1-28, wherein the first polypeptide further comprises one or more nuclear localization sequences (NLSs).

30. The system of claim 29, wherein the first polypeptide comprises a C-terminal and an N-terminal NLS.

31. The system of claim 30, further comprising a peptide linker between the N-terminal NLS and the DNA binding protein.

32. The system of claim 30 or 31, further comprising a peptide linker between the C-terminal NLS and the first binding moiety.

33. The system of any one of claims 1-32, wherein the second polypeptide further comprises one or more nuclear localization sequences (NLSs).

34. The system of claim 33, wherein the second polypeptide comprises a C-terminal and an N-terminal NLS.

35. The system of claim 34, further comprising a peptide linker between the C-terminal NLS and the DNA polymerase domain.

36. The system of claim 33 or 34, further comprising a peptide linker between the N-terminal NLS and the second binding moiety.

37. The system of any one of claims 29-36, wherein the NLSs have, individually, a sequence selected from Table 3 or a sequence having one or two substitutions relative to a sequence from Table 3.

38. The system of any one of claims 31-36, wherein the peptide linkers have, individually, a sequence selected from Table 15 or a sequence having at least 80%, 85%, 90%, 95%, 96%, 97%, 98%, or 99% identity with a sequence from Table 15.

39. The system of any one of claims 1-38, wherein the first polypeptide and the second polypeptide comprise compatible sequences from Table 21 or Table 20 or sequences having at least 80%, 85%, 90%, 95%, 96%, 97%, 98%, or 99% identity with compatible sequence from Table 21 or Table 20.

40. The system of any one of claims 1-39, further comprising a self-cleaving peptide joining the first polypeptide to the second polypeptide.

41. The system of claim 40, wherein the self-cleaving peptide comprises a sequence from Table 19 or a sequence having one or two substitutions relative to a sequence from Table 19.

42. The system of claim 40, wherein the self-cleaving peptide comprises SEQ ID NO: 8004.

43. The system of any one of claims 40-42, comprising a sequence having 80%, 85%, 90%, 95%, 96%, 97%, 98%, or 99% identity relative to a sequence from Table 18.

44. The system of any one of claims 40-42, comprising a sequence selected from Table 18.

45. The system of claim 43 or 44, wherein the sequence from Table 18 is SEQ ID NO: 8005.

46. A prime editor system comprising a split prime editor comprising a DNA binding domain and a DNA polymerase domain, wherein the split prime editor comprises a first polypeptide comprising a first amino acid sequence and a second polypeptide comprising a second amino acid sequence.

47. The prime editor system of claim 46, wherein the first amino acid sequence forms at least a portion of the DNA binding domain.

48. The prime editor system of claim 46 or claim 47, wherein the second amino acid sequence forms at least a portion of the DNA polymerase domain.

49. The prime editor system of claim 47 or claim 48, wherein the first amino acid sequence forms the DNA binding domain.

50. The prime editor system of claim 49, wherein the first amino acid sequence forms the DNA binding domain and a portion of the DNA polymerase domain.

51. The prime editor system of claim 47 or claim 48, wherein the second amino acid sequence forms the DNA polymerase domain.

52. The prime editor system of claim 51, wherein the second amino acid sequence forms the DNA polymerase domain and a portion of the DNA binding domain.

53. The prime editor system of claim 46, wherein the first amino acid sequence forms at least a portion of the DNA polymerase domain.

54. The prime editor system of claim 46 or claim 53, wherein the second amino acid sequence forms at least a portion of the DNA binding domain.

55. The prime editor system of claim 53 or claim 54, wherein the first amino acid sequence forms the DNA polymerase domain.

56. The prime editor system of claim 55, wherein the first amino acid sequence forms the DNA polymerase domain and a portion of the DNA binding domain.

57. The prime editor system of claim 53 or claim 54, wherein the second amino acid sequence forms the DNA binding domain.

58. The prime editor system of claim 57, wherein the second amino acid sequence forms the DNA binding domain and a portion of the DNA polymerase domain.

59. The prime editor system of any one of claims 46 to 58, wherein the first polypeptide and the second polypeptide are configured to passively assemble in a host cell to form the split prime editor.

60. The prime editor system of any one of claims 46 to 58, wherein the first polypeptide has affinity for the second polypeptide.

61. The prime editor system of any one of claims 46 to 58, wherein the second polypeptide has affinity for the first polypeptide.

62. The prime editor system of claim 60 or claim 61, wherein the first polypeptide comprises a single-domain antibody.

63. The prime editor system of claim 62, wherein the single-domain antibody comprises an amino acid sequence as set forth in Table 17.

64. The prime editor system of claim 62 or claim 63, wherein the second polypeptide comprises a peptide tag that is configured to be bound by the single domain antibody.

65. The prime editor system of claim 64, wherein the peptide tag comprises a SpotTag® or a BC2 tag.

66. The prime editor system of claim 64, wherein the peptide tag comprises an amino acid sequence as set forth in Table 16.

67. The prime editor system of claim 60 or 61, wherein the first polypeptide comprises a peptide tag that is configured to bind to a single domain antibody.

68. The prime editor system of claim 67, wherein the peptide tag comprises a SpotTag® or a BC2 tag.

69. The prime editor system of claim 67, wherein the peptide tag comprises an amino acid sequence as set forth in Table 16.

70. The prime editor system of any one of claims 67 to 69, wherein the second polypeptide comprises a single-domain antibody.

71. The prime editor system of claim 70, wherein the single-domain antibody comprises an amino acid sequence as set forth in Table 17.

72. The prime editor system of any one of claims 62 to 71, wherein the single-domain antibody is a NANOBODY®.

73. The prime editor system of any one of claims 46 to 58, wherein the split prime editor further comprises an affinity moiety that has affinity for either the DNA binding domain or the DNA polymerase domain.

74. The prime editor system of claim 73, wherein the affinity moiety has affinity for the DNA binding domain.

75. The prime editor system of claim 73, wherein the affinity moiety has affinity for the DNA polymerase domain.

76. The prime editor system of claim 73, wherein the DNA binding domain comprises a peptide tag that is configured to bind to the affinity moiety and the DNA polymerase domain comprises the affinity moiety.

77. The prime editor system of claim 73, wherein the DNA binding domain comprises the affinity moiety and the DNA polymerase domain comprises a peptide tag that is configured to bind to the affinity moiety.

78. The prime editor system of any one of claims 73-77, wherein the affinity moiety comprises an antibody or fragment thereof.

79. The prime editor system of any one of claims 73-78, wherein the affinity moiety comprises a single-domain antibody.

80. The prime editor system of claim 79, wherein the single-domain antibody or fragment thereof is a NANOBODY®.

81. The prime editor system of claim 79 or claim 80, wherein the single-domain antibody comprises any one of the amino acid sequences as set forth in Table 17.

82. The prime editor system of any one of claims 73 to 75, wherein the affinity moiety is fused to the first polypeptide and has affinity for the second amino acid sequence.

83. The prime editor system of any one of claims 73 to 75, wherein the affinity moiety is fused to the second polypeptide and has affinity for the first amino acid sequence.

84. The prime editor system of any one of claims 1 to 73, wherein the first polypeptide comprises a C-terminal intein sequence.

85. The prime editor system of claim 84, wherein the second polypeptide comprises a N-terminal intein sequence.

86. The prime editor system of claim 85, wherein assembly of the first polypeptide and the second polypeptide in a host cell results in fusion of the C-terminal intein sequence and the N-terminal intein sequence to generate a full intein sequence, which then results in splicing and excision of the full intein sequence.

87. The prime editor system of any one of claims 46 to 58, wherein the first polypeptide comprises a first affinity moiety and the second polypeptide comprises a second affinity moiety.

88. The prime editor system of claim 87, wherein the first affinity moiety has affinity for the second affinity moiety.

89. The prime editor system of claim 87 or claim 88, wherein the first affinity moiety comprises a C-terminal leucine zipper monomer.

90. The prime editor system of claim 89, wherein the second affinity moiety comprises an N-terminal leucine zipper monomer.

91. The prime editor system of claim 90, wherein the C-terminal leucine zipper monomer and the N-terminal leucine zipper monomer forms a dimer in a host cell.

92. The prime editor system of claim 87 or 88, wherein the first affinity moiety comprises a C-terminal dimerization domain.

93. The prime editor system of claim 92, wherein the second affinity moiety comprises a N-terminal dimerization domain.

94. The prime editor system of claim 93, wherein the C-terminal dimerization domain and the N-terminal dimerization domain form a dimer in a host cell.

95. The prime editor system of any one of claims 46 to 94, wherein the prime editor system comprises a scaffold RNA.

96. The prime editor system of claim 95, wherein the first polypeptide and/or the second polypeptide comprises an adapter protein that has affinity for the scaffold RNA.

97. The prime editor system of claim 96, wherein the adapter protein is selected from one or more of a MS2 coat/adapter protein (MCP), a PP7 adapter protein, a Qβ adapter protein, a F2 adapter protein, a GA adapter protein, a fr adapter protein, a JP501 adapter protein, a M12 adapter protein, a R17 adapter protein, a BZ13 adapter protein, a JP34 adapter protein, a JP500 adapter protein, a KU1 adapter protein, a M11 adapter protein, a MX1 adapter protein, a TW18 adapter protein, a VK adapter protein, a SP adapter protein, a FI adapter protein, a ID2 adapter protein, a NL95 adapter protein, a TW19 adapter protein, a AP205 adapter protein, a ϕCb5 adapter protein, a ϕCb8r adapter protein, a ϕ12r adapter protein, a ϕCb23r adapter protein, a 7s adapter protein and a PRR1 adapter protein.

98. The prime editor system of any one of claims 46 to 58, further comprising a scaffold protein that has affinity for the first polypeptide and/or the second polypeptide.

99. The prime editor system of claim 98, wherein the scaffold protein is fused to the first polypeptide or the second polypeptide.

100. The prime editor system of claim 98, wherein the scaffold protein is not fused to either the first polypeptide or the second polypeptide.

101. The prime editor system of any one of claims 98 to 100, further comprising a second scaffold protein that has affinity for the scaffold protein.

102. The prime editor system of claim 101, wherein the second scaffold protein has affinity for the first polypeptide.

103. The prime editor system of claim 101 or 102, wherein the second scaffold protein has affinity for to the second polypeptide.

104. The prime editor system of any one of claims 101 to 103, wherein the second scaffold protein is fused to the first polypeptide or the second polypeptide.

105. The prime editor system of any one of claims 101 to 104, wherein the second scaffold protein is not fused to either the first polypeptide or the second polypeptide.

106. The prime editor system of any one of claims 46 to 58, wherein the first polypeptide has affinity for an endogenous protein in a host cell.

107. The prime editor system of claim 106, wherein the second polypeptide has affinity for the endogenous protein in a host cell.

108. The prime editor system of any one of claims 46 to 58, wherein the first polypeptide has affinity for a first endogenous protein in a host cell and the second polypeptide has affinity for a second endogenous protein in a host cell, and the first endogenous protein has affinity for the second endogenous protein.

109. The prime editor system of any one of claims 46 to 58, wherein the first polypeptide is configured to become covalently attached to the second polypeptide in a host cell.

110. The prime editor system of claim 109, wherein the first polypeptide comprises a SpyTag peptide sequence and the second polypeptide comprises a SpyCatcher peptide sequence.

111. The prime editor system of claim 109, wherein the first polypeptide comprises a SnoopTag peptide sequence and the second polypeptide comprises a SnoopCatcher peptide sequence.

112. The prime editor system of claim 109, wherein the first polypeptide comprises a SdyTag peptide sequence and the second polypeptide comprises a SdyCatcher peptide sequence.

113. The prime editor system of claim 109, wherein the first polypeptide comprises a DogTag peptide sequence and the second polypeptide comprises a DogCatcher peptide sequence.

114. The prime editor system of claim 109, wherein the first polypeptide comprises a SpyTag peptide sequence and the second polypeptide comprises a SpyDock peptide sequence.

115. The prime editor system of claim 109, wherein the first polypeptide comprises an isopeptag peptide sequence and the second polypeptide comprises a Pilin-C peptide sequence.

116. The prime editor system of any one of claims 46-115, wherein the split prime editor comprises a third polypeptide encoding a third amino acid sequence.

117. The prime editor system of claim 116, wherein the third amino acid sequence forms at least a portion of the DNA binding domain and/or the DNA polymerase domain.

118. The prime editor system of any one of claims 46 to 117, wherein the DNA binding domain comprises a CRISPR associated (Cas) protein domain.

119. The prime editor system of claim 118, wherein the Cas protein domain has nickase activity.

120. The prime editor system of claim 119, wherein the Cas protein domain is a Cas9.

121. The prime editor system of claim 120, wherein the Cas9 comprises a mutation in an HNH domain.

122. The prime editor system of claim 120, wherein the Cas9 comprises a H840A mutation in the HNH domain.

123. The prime editor system of claim 118, wherein the Cas protein domain is a Cas12b.

124. The prime editor system of claim 118, wherein the Cas protein domain is a Cas12a, Cas12b, Cas12c, Cas12d, Cas12e, Cas14a, Cas14b, Cas14c, Cas14d, Cas14e, Cas14f, Cas14g, Cas14h, Cas14u, or a Casφ.

125. The prime editor system of claim 118, wherein the Cas protein domain comprises any one of the amino acid sequences as set forth in Table 14.

126. The prime editor system of any one of claims 46 to 125, wherein the DNA polymerase domain comprises a reverse transcriptase.

127. The prime editor system of claim 126, wherein the reverse transcriptase is a retrovirus reverse transcriptase.

128. The prime editor system of claim 126, wherein the reverse transcriptase is a Moloney murine leukemia virus (M-MLV) reverse transcriptase.

129. The prime editor system of claim 126, wherein the reverse transcriptase comprises any one of the sequences as set forth in Table 11, Table 12, or Table 13.

130. The prime editor system of any one of claims 46 to 129, wherein the first polypeptide comprises at least one peptide linker.

131. The prime editor system of claim 130, wherein the first polypeptide comprises at least two peptide linkers.

132. The prime editor system of any one of claims 46 to 131, wherein the second polypeptide comprises at least one peptide linker.

133. The prime editor system of claim 132, wherein the second polypeptide comprises at least two peptide linkers.

134. The prime editor system of claim 130 or 132, wherein the at least one peptide linker comprises 5 to 100 amino acids.

135. The prime editor system of claim 130 or 132, wherein the at least one peptide linker comprises an amino acid sequence as set forth in Table 15.

136. The prime editor system of any one of claims 46 to 135, wherein the first polypeptide further comprises at least one nuclear localization sequence.

137. The prime editor system of any one of claims 46 to 135, wherein the second polypeptide further comprises at least one nuclear localization sequence.

138. The prime editor system of claim 136 or 137, wherein the at least one nuclear localization sequence comprises an amino acid sequence as set forth in Table 3.

139. The prime editor system of any one of claims 46 to 138, wherein the first polypeptide and the second polypeptide are joined by a self-cleaving peptide.

140. The prime editor system of claim 139, wherein the self-cleaving peptide is a P2A peptide.

141. The prime editor system of claim 140, wherein the P2A peptide comprises a sequence set forth in SEQ ID NO: 8004.

142. The prime editor system of claim 141, wherein the prime editor comprises an amino acid sequence as set forth in Table 18.

143. A lipid nanoparticle (LNP) or ribonucleoprotein (RNP) comprising the prime editing system of any one of claims 46 to 142, or a component thereof.

144. A polynucleotide encoding the prime editor of any one of claims 46 to 142.

145. The polynucleotide of claim 144, wherein the polynucleotide is operably linked to a regulatory element.

146. The polynucleotide of claim 145, wherein the regulatory element is an inducible regulatory element.

147. A vector comprising the polynucleotide of any one of claims 144 to 146.

148. The vector of claim 147, wherein the vector is an AAV vector.

149. A polynucleotide encoding the first polypeptide of any one of claims 46 to 142.

150. The polynucleotide of claim 149, wherein the polynucleotide is operably linked to a regulatory element.

151. The polynucleotide of claim 150, wherein the regulatory element is an inducible regulatory element.

152. A vector comprising the polynucleotide of any one of claims 144 to 151.

153. The vector of claim 152, wherein the vector is an AAV vector, such as a trans-splicing vector.

154. A polynucleotide encoding the second polypeptide of any one of claims 46 to 142.

155. The polynucleotide of claim 154, wherein the polynucleotide is operably linked to a regulatory element.

156. The polynucleotide of claim 155, wherein the regulatory element is an inducible regulatory element.

157. A vector comprising the polynucleotide of any one of claims 154 to 156.

158. The vector of claim 157, wherein the vector is an AAV vector, such as a trans-splicing vector.

159. A kit comprising a first polynucleotide and a second polynucleotide, wherein the first polynucleotide is a polynucleotide of any one of claims 149-151 and the second polynucleotide is a polynucleotide of any one of claims 154-156.

160. The kit of claim 159, wherein the first polynucleotide and/or the second polynucleotide is in a vector.

161. The kit of claim 160, wherein the vector is an AAV vector.

162. The kit of claim 161, wherein the vector is an AAV trans-splicing vector.

163. An isolated cell comprising the prime editor system of any one of claims 1 to 142, the LNP or RNP of claim 143, the polynucleotide of any one of claims 144 to 146, 149 to 151, or 154 to 156, or the vector of any one of claim 147-148, 152-153, or 157-158.

164. The isolated cell of claim 163, wherein the cell is a human cell.

165. A pharmaceutical composition comprising i) the prime editor system of any one of claims 1 to 142, the LNP or RNP of claim 143, the polynucleotide of any one of claims 144 to 146, 149 to 151, or 154 to 156, or the vector of any one of claim 147-148, 152-153, or 157-158; and (ii) a pharmaceutically acceptable carrier.

166. The prime editor system of any one of claims 1-142, further comprising a prime editor guide RNA (a PERNA).

167. A method for editing a gene, the method comprising contacting the gene with a prime editor system of claim 166, wherein the PEgRNA directs the prime editor to incorporate the intended nucleotide edit in the gene, thereby editing the gene.

168. The method of claim 167, wherein the prime editor synthesizes a single stranded DNA encoded by an editing template, wherein the single stranded DNA replaces an editing target sequence and results in incorporation of the intended nucleotide edit into a region corresponding to the editing target sequence in the gene.

169. The method of claim 167 or 168, wherein the gene is in a cell.

170. The method of claim 169, wherein the cell is a mammalian cell.

171. The method of claim 169, wherein the cell is a human cell.

172. The method of any one of claims 169-171, wherein the cell is in a subject.

173. The method of claim 172, wherein the subject is a human.

174. The method of any one of claims 169-171, further comprising administering the cell to a subject after incorporation of the intended nucleotide edit.
